(19) United States
(12) Patent Application Publication
(10) Pub. No.: US 2024/0182460 A1
(43) Pub. Date: Jun. 6, 2024

(54) ALLOSTERIC CHROMENONE INHIBITORS OF PHOSPHOINOSITIDE 3-KINASE (PI3K) FOR THE TREATMENT OF DISEASE

(71) Applicant: PETRA PHARMA CORPORATION, New York, NY (US)

(72) Inventors: Surendra DAWADI, Longmont, CO (US); Hua-Yu WANG, Chula Vista, CA (US); Marcos SAINZ, San Diego, CA (US); Gabrielle R. KOLAKOWSKI, Durango, CO (US); Nathan Edward WRIGHT, San Diego, CA (US); Christopher Glenn MAYNE, Boulder, CO (US); Gerit Maria POTOTSCHNIG, San Diego, CA (US); Erin ANDERSON, Boulder, CO (US); Warren Christopher CHAN, San Diego, CA (US); Shuai CHEN, San Diego, CA (US); Manoj KUMAR, Broomfield, CO (US); Kyunga LEE, San Diego, CA (US); Ganesh MURHADE, Louisville, CO (US); Mariam Nader SALIB, Escondido, CO (US); Latoya Denise SCAGGS, Broomfield, CO (US); Xia WANG, San Diego, CA (US); Thomas IRVIN, Erie, CO (US); Alfredo PICADO VALENZUELA, Louisville, CO (US); Katelyn LONG, Louisville, CO (US); Carina JETTE, San Diego, CA (US)

(21) Appl. No.: 18/498,956

(22) Filed: Oct. 31, 2023

Related U.S. Application Data
(60) Provisional application No. 63/421,277, filed on Nov. 1, 2022.
(60) Provisional application No. 63/496,280, filed on Apr. 14, 2023.
(60) Provisional application No. 63/511,400, filed on Jun. 30, 2023.
(60) Provisional application No. 63/591,725, filed on Oct. 19, 2023.

(51) Int. Cl. C07D 413/14; C07D 311/22; C07D 311/30; C07D 405/04; C07D 405/12; C07D 413/12; C07D 417/14; C07D 471/04; C07D 487/04; C07D 491/048; C07D 491/056; C07D 493/08; C07D 498/04; C07D 513/04

(52) U.S. Cl. CPC C07D 413/14; C07D 311/22; C07D 311/30; C07D 405/04; C07D 405/12; C07D 413/12; C07D 417/14; C07D 471/04; C07D 487/04; C07D 491/048; C07D 491/056; C07D 493/08; C07D 498/04; C07D 513/04

(57) ABSTRACT

The disclosure relates to compounds of Formula (I) as allosteric chromenone inhibitors of phosphoinositide 3-kinase (PI3K) useful in the treatment of diseases or disorders associated with PI3K modulation, Formula (I):

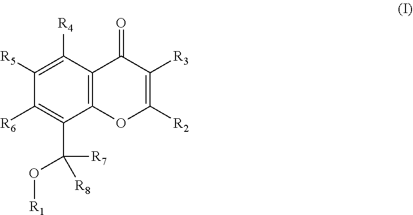

or pharmaceutically acceptable salts thereof wherein R₁, R₂, R₃, R₄, R₅, R₆, R₇, and R₈, are as defined herein. The disclosure also relates to methods of making and using compounds of Formula (I) or pharmaceutically acceptable salts thereof.

### CROSS-REFERENCE TO RELATED PATENT APPLICATIONS

The present application claims the benefit of priority to U.S. Provisional Application No. 63/591,725, filed on Oct. 19, 2023; U.S. Provisional Application No. 63/511,400, filed on Jun. 30, 2023; U.S. Provisional Application No. 63/496,280, filed on Apr. 14, 2023; and U.S. Provisional Application No. 63/421,277, filed on Nov. 1, 2022; the contents of which are incorporated herein by reference in their entireties.

### FIELD

The present invention is directed to allosteric chromenone inhibitors of phosphoinositide 3-kinase (PI3K) useful in the treatment of diseases, or disorders associated with PI3K modulation. The invention is directed toward compounds and compositions which inhibit PI3K, methods of (or uses for) treating a disease, or disorder associated with PI3K (e.g., CLOVES syndrome (congenital lipomatous overgrowth, vascular malformations, epidermal naevi, scoliosis/skeletal, and spinal syndrome), PIK3CA-related overgrowth syndrome (PROS), breast cancer, brain cancer, prostate cancer, endometrial cancer, gastric cancer, leukemia, lymphoma, sarcoma, colorectal cancer, lung cancer, ovarian cancer, skin cancer, or head and neck cancer), and using, or methods of using, PI3K inhibitors in combination with one or more additional cancer therapies.

### BACKGROUND

The phosphoinositide 3-kinases (PI3Ks) signaling pathway is one of the most highly mutated systems in human cancers. PI3K signaling is involved in many other disease states including allergic contact dermatitis, rheumatoid arthritis, osteoarthritis, inflammatory bowel diseases, chronic obstructive pulmonary disorder, psoriasis, multiple sclerosis, asthma, disorders related to diabetic complications, and inflammatory complications of the cardiovascular system such as acute coronary syndrome.

PI3Ks are members of a unique, and conserved family of intracellular lipid kinases that phosphorylate the 3′-OH group on phosphatidylinositols, or phosphoinositides. The PI3K family comprises 15 kinases with distinct substrate specificities, expression patterns, and modes of regulation (Katso et al., Annu Rev Cell Dev Biol. 2001; 17:615-75). The class I PI3Ks (p110α, p110β, p110δ, and p110γ) are typically activated by tyrosine kinases, or G-protein coupled receptors to generate PIP3, which engages downstream effectors such as those in the pathways of Akt/PDK1, mTOR, the Tec family kinases, and the Rho family GTPases. The class II, and III PI3Ks play a key role in intracellular trafficking through the synthesis of PI(3)P, and PI(3,4)P2.

The PI3K isoforms have been implicated, for example, in a variety of human cancers, and disorders. Mutations in the gene coding for PI3K isoforms, or mutations which lead to upregulation of a PI3K isoform are believed to occur in many human cancers. Mutations in the gene coding for a PI3K isoform are point mutations clustered within several hotspots in helical, and kinase domains. Because of the high rate of PI3K mutations, targeting of this pathway may provide valuable therapeutic opportunities.

Genetic alterations in genes in PI3K signaling are believed to be involved in a range of cancers such as endometrial cancer, breast cancer, esophageal squamous-cell cancer, cervical squamous-cell carcinoma, cervical adenocarcinoma, colorectal adenocarcinoma, bladder urothelial carcinoma, glioblastoma, ovarian cancer, non-small-cell lung cancer, esophagogastric cancer, nerve-sheath tumor, head and neck squamous-cell carcinoma, melanoma, esophagogastric adenocarcinoma, soft-tissue sarcoma, prostate cancer, fibrolamellar carcinoma, hepatocellular carcinoma, diffuse glioma, colorectal cancer, pancreatic cancer, cholangiocarcinoma, B-cell lymphoma, mesothelioma, adrenocortical carcinoma, renal non-clear-cell carcinoma, renal clear-cell carcinoma, germ-cell carcinoma, thymic tumor, pheochromocytoma, miscellaneous neuroepithelial tumor, thyroid cancer, leukemia, and encapsulated glioma (Goncalves M D, Hopkins B D, Cantley L C. Phosphatidylinositol 3-Kinase, Growth Disorders, and Cancer. N Engl J Med. 2018 Nov. 22; 379(21):2052-2062).

The alpha (α) isoform of PI3K has been implicated, for example, in a variety of human cancers. Angiogenesis has been shown to selectively require the a isoform of PI3K in the control of endothelial cell migration. (Graupera et al, Nature 2008; 453; 662-6). Mutations in the gene coding for PI3Kα, or mutations which lead to upregulation of PI3Kα are believed to occur in many human cancers such as lung, stomach, endometrial, ovarian, bladder, breast, colon, brain, prostate, and skin cancers. Mutations in the gene coding for PI3Kα are point mutations clustered within several hotspots in helical, and kinase domains, such as E542K, E545K, and H1047R. Many of these mutations have been shown to be oncogenic gain-of-function mutations. Because of the high rate of PI3Kα mutations, targeting of this pathway may provide valuable therapeutic opportunities. While other PI3K isoforms such as PI3Kδ, or PI3Kγ are expressed primarily in hematopoietic cells, PI3Kα, along with PI3Kβ, is expressed constitutively.

Mutated PI3Kα has been implicated in brain metastases in HR+/HER2− metastatic breast cancers. Development of brain-penetrant PI3Kα inhibitors may provide improved therapeutic benefit over current PI3Kα inhibitors. (Fitzgerald et al., Association between PIK3CA mutation status and development of brain metastases in HR+/HER2− metastatic breast cancer. Ann Oncol 30:v110, 2019 (suppl 5)).

Due to the central role of PI3Kα in regulating organismal glucose homeostasis, PI3K inhibition in patients often gives rise to hyperglycemia and/or hyperinsulinemia (Busaidy N L, et al, Management of metabolic effects associated with anticancer agents targeting the PI3K-Akt-mTOR pathway. J Clin Oncol 2012; 30:2919-28). High levels of circulating insulin could potentially be mitogenic and/or antiapoptotic for cancer cells, and thus negate the antiproliferative effects of PI3K inhibitors (Blouin M-J, et al, Abstract 4615: the hyperinsulinemia caused by PI3K inhibitors attenuates their antineoplastic efficacy, but can be minimized by co-administration of metformin. Cancer Res 2013; 73:4615).

In the setting of cancer with mutated PI3Kα, one way to overcome the problem of compensatory production of insulin and/or glucose upon systemic PI3Kα inhibition would be to develop inhibitors with enhanced selectivity for mutant PI3Kα over wild-type PI3Kα. This would create an increased window for drug dosing to selectively inhibit the pathologic signaling of mutant PI3Kα in the cancer cells without affecting the wild-type PI3Kα in the host tissues that control systemic metabolism (Okkenhaug K, Graupera M, Vanhaesebroeck B. Targeting PI3K in Cancer: Impact on Tumor Cells, Their Protective Stroma, Angiogenesis, and Immunotherapy. Cancer Discov. 2016 October; 6(10):1090-1105), thus limiting toxicities, and permitting higher doses, and more complete inhibition of the drug target (Ariella B. Hanker, et al, Challenges for the clinical development of PI3K inhibitors: Strategies to improve their impact in solid tumors. Cancer Discov. 2019 April; 9(4): 482-491).

Currently PI3Kα inhibitors are nearly equipotent to wild-type, and mutant PI3Kα. Mutant selective inhibitors have been elusive due to the PI3Kα mutations location far from the active site.

As such, inhibitors which target a second, peripheral binding pocket near a known mutation (e.g., H1047R) may provide a route to selective PI3Kα inhibition. Thus, targeting a mutated, peripheral binding pocket of PI3Kα, provides a valuable therapeutic target for drug development.

As such, kinase inhibitors of lipid kinases such as PI3Kα are prime areas for drug development. One goal is to develop PI3Kα inhibitors that exhibit suitable potency for PI3Kα 's. One goal is to develop PI3Kα inhibitors that exhibit suitable potency for PI3Kα 's that are associated with metastatic disease such as PI3Kα(H1047R) and PI3Kα(E545K). Another goal is to develop PI3Kα inhibitors that exhibit suitable selectivity versus wild-type for mutant PI3Kα 's that are associated with metastatic disease including PI3Kα(H1047R) and PI3Kα(E545K). Another goal is to develop PI3Kα inhibitors that exhibit metabolic stability. A further goal is to develop PI3Kα inhibitors that do not substantially modify the expression of cytochrome P450 enzymes, such as the expression of CYP3A4, where the modified expression of a cytochrome P450 enzyme may affect the metabolic stability of the PI3Kα inhibitors or may affect the metabolic stability of another drug and result in a drug-drug interactions (DDI). Another goal is to develop PI3Kα inhibitors that do not cause adverse events (AEs) such as hyperglycemia, hyperinsulinemia, and diarrhea when the PI3Kα inhibitors are administered at an effective dose for inhibiting the activity of PI3Kα and treating a disease or disorder associated with PI3Kα activity. Another goal is to develop PI3Kα inhibitors that do not exhibit substantial activity against targets other than PI3Kα, including other kinases other than PI3Kα.

### SUMMARY

In one aspect, the present invention relates to compound of Formula (I):

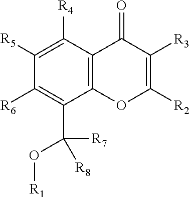

or pharmaceutically acceptable salt thereof, wherein:
R₁ is an optionally substituted bicyclic ring selected from isobenzofuranone, benzofuranone, isoindolinone, indolinone, quinazolinone, 3,4-dihydro-2H-isoquinolin-1-one, 2H-isoquinolin-1-one, imidaza[1,2-a]pyridine, or benzothiazolone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
R₁ is a group of the formula:

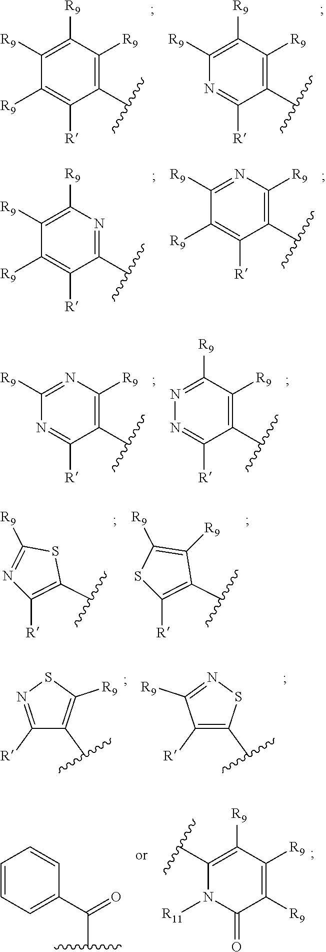

R′ is hydrogen, halogen, C₁-C₃ alkyl, C₁-C₃ haloalkyl, C₁-C₃ hydroxyalkyl, C₁-C₃ alkoxy, C₁-C₃ haloalkoxy, —OH, —CH(OH)—CH₂OH, —CH(OH)C₁-C₃ haloalkyl, —CO—CH₂OH, C₃-C₆ cycloalkyl, —NO₂, —NR₁₁R₁₁, —N(R₁₁)—CO₂C₁-C₃ alkyl, —N(R)—SO₂C₁-C₃ alkyl, —N(R)—SO₂R₁₅, —SO₂C₁-C₃ alkyl, —SOC₁-C₃ alkyl, —SO₂NR₁₁R₁₁, —SO₂NR₁₁R₁₂, —SO₂N(R)—CO—C₁-C₃ alkyl, —SO₂N(R₁₁)—CN, —C(═N—OH)—NH₂, —CN, —CONR₁₁R₁₂, —CON(R₁₁)—(CH₂)ₙ—R₁₃, —CO—SR₁₂, —CO—NHSO₂R₁₆, —COCH═SOR₁₁(R₁₁), or —COCH₂CN, or a group of the formula:

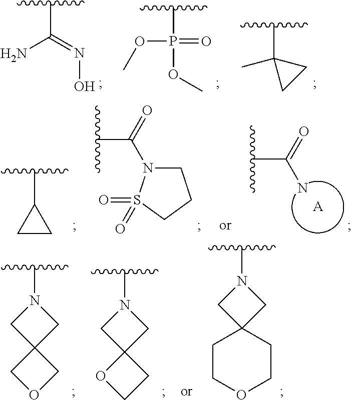

wherein ring A is pyrrolidine optionally substituted with a —CN; or
R′ is selected from oxetane, azetidine, pyrrolidine, tetrahydrofuran, morpholine, thiomorpholine, piperidine, piperazine, pyrrole, furan, thiophene, pyrazole, imidazole, isoxazole, oxazole, isothiazole, thiazole, triazole, oxadiazole, oxadiazolinone (e.g., 1,2,4-oxadiazolin-5-one), 1,4-oxazepane, thiadiazole, tetrazole, phenyl, pyridine, pyridazine, pyrimidine, pyrazine, oxazepane, or triazine; each of which is optionally substituted with one to three substituents independently selected from oxo, —OH, —NR₁₁R₁₁, —N(R₁₁)—CO—R₁₁, —N(R₁₁)—CN, —OR₁₁, —CN, halogen, morpholino, oxetane, C₁-C₆ haloalkyl or C₁-C₆ alkyl optionally substituted with —OH, an aryl, a 5-member heteroaryl or a 6-member heteroaryl; or
R′ is a group of the formula:

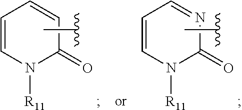

R₂ is a group of the formula:

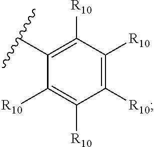

R₂ is a group of the formula:

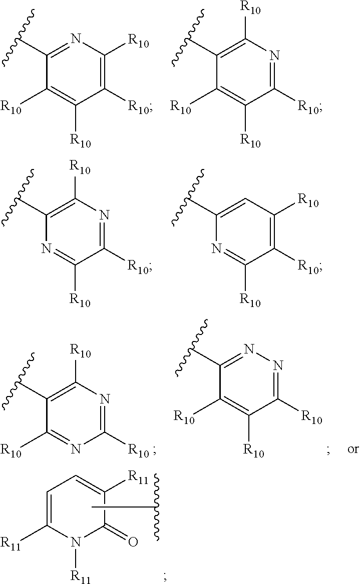

or
R₂ is an optionally substituted 5-member ring heteroaryl selected from pyrrole, furan, thiophene, pyrazole, isoxazole, isothiazole, imidazole, oxazole, thiazole, triazole, tetrazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —OH, —NR₁₁R₁₁, —NR₁₁CO₂R₁₁, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH (e.g., C₁-C₆ hydroxyalkyl such as —CH₂—CH₂—OH and optionally comprising a secondary —OH such as —CH₂—CH(CH₃)—OH or a tertiary —OH such as —CH₂—C(CH₃)₂—OH), halogen, oxetanyl, 2-oxabicyclo[2.1.1]hexane, C₁-C₃ alkoxy, an optionally substituted C₃-C₅ cycloalkyl, —CONR₁₁R₁₁, or —S(O)₂CH₃; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or R₂ is an optionally substituted bicyclic ring selected from 1,3-benzodioxole, 2,3-dihydro-1,4-benzodioxine, quinoxaline, quinazoline, indole, indazole, isoindazole, benzimidazole, benzotriazole, benzothiazole, benzoxazole, benzotriazole, benzofuran, benzofurazan, pyridofurazan, quinoline, 1,5-naphthyridine, isoindolin-1-one, indolin-2-one, benzomorpholine, benzo[d]oxazol-2(3H)-one, imidazo[1,2-a]pyridine, 1,3-dihydro-2H-pyrrolo[2,3-b]pyridin-2-one, [1,3]dioxolo[4,5-b]pyridine, 6,7-dihydro-4H-pyrazolo[5,1-c][1,4]oxazine, 1,2,3,4-tetrahydroisoquinoline, 4,5,6,7-tetrahydropyrazolo[1,5-a]pyrazine, pyrazolo[4,3-b]pyridine, pyrazolo[3,4-b]pyridine, pyrazolo[3,4-c]pyridine, pyrazolo[1,5-a]pyrimidine, oxazolo[4,5-b]pyridine, oxazolo[5,4-b]pyridine, thiazolo[5,4-b]pyridine, 2,1,3-benzothiadiazole, imidazo[1,2-a]pyrimidine, or 2,3-dihydro-[1,4]dioxino[2,3-b]pyridine, 6,7-dihydro-5H-cyclopent[b]pyridine, 5,6-dihydro-4H-pyrrolo[1,2-b]pyrazole, 3,4-dihydro-2(1H)quinolinone, 2H-1,4-benzoxazin-3(4H)-one, 2-hydroxyquinoline, 3,4-dihydroisoquinolin-1,(2H)-one, 1-hydroxyisoquinoline, 1,4-dihydro-3(2H)-isoquinolinone, 3H-imidazo[4,5-b]pyridine, 4,5-dihydro-7H-pyrazolo[1,5-c]1,3]oxazine, 6,7-dihydro-5H-pyrazolo[5,1-b][1,3]oxazine, furo[3,2-c]pyridine, furo[3,2-b]pyridine, 2,3-dihydropyrazolo[5,1-b]oxazole, 5H,6H,8H-imidazo[2,1-c][1,4]oxazine, pyrazolo[1,5-b]pyridazine, imidazo[1,2-b]pyridazine, 2,4-dihydro-1H-benzo[d][1,3]oxazine, 5-oxaspiro[2,3]hexane, imidazo[1,5-a]pyridine, pyrazolo[1,5-a]pyridine; or an optionally substituted bicyclic heteroaryl of 8 to 10 ring atoms containing 1, 2, 3, 4, or 5 ring heteroatoms independently selected from N, O, or S; wherein the optionally substituted bicyclic ring is optionally substituted with one to three substituents each independently selected from halogen and C₁-C₆ alkyl; or wherein the optionally substituted bicyclic ring or the optionally substituted bicyclic heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —NR₁₁R₁₁, —NR₁₁CO₂R₁₁, —NR₁₁C(O)R₁₁, —OH, oxetanyl, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH (e.g., C₁-C₆ hydroxyalkyl such as —CH₂—CH₂—OH and optionally comprising a secondary —OH such as —CH₂—CH(CH₃)—OH or a tertiary —OH such as —CH₂—C(CH₃)₂—OH), oxetanyl, C₁-C₃ alkoxy, —CONR₁₁R₁₁, phenyl, or an optionally substituted C₃-C₅ cycloalkyl; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is a cyclopropyl, cyclobutyl, cyclopentyl, bicyclo[1.1.1]pentane, bicyclo[2.2.2]octane, or cyclohexyl, each optionally substituted with one to three R₁₀ substituents, or R₂ is a group of the formula:

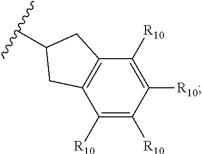

R₂ is a heterocycloalkyl, and optionally oxetane substituted with one to three R₁₀ substituents;
R₃ is —H, halogen, —CN, —C(CN)═CHOH, —N(H)(C₁-C₃ alkyl), —N(C₁-C₃ alkyl)₂, —N(H)(CH₂CH₂CO₂H), —CO—C₁-C₃ alkyl, C₁-C₆ alkyl, C₁-C₆ haloalkyl, C₁-C₆ hydroxyalkyl, C₃-C₅ cycloalkyl, an optionally substituted heterocycle of 3 to 5 ring atoms containing 1, 2, or 3 ring heteroatoms independently selected from N, O, or S, or an optionally substituted heteroaryl of 5 or 6 ring atoms containing 1, 2, or 3 ring heteroatoms independently selected from N, O, or S; wherein the optionally substituted heterocycle or heteroaryl is each optionally substituted with one to three substituents each independently selected from halogen, C₁-C₃ alkyl, or C₁-C₃ haloalkyl;
each of R₄, R₅ and R₆ is independently —H, halogen, —CN, C₁-C₆ alkyl or C₁-C₆ haloalkyl;
R₇ is —CN, C₁-C₆ alkyl, CH₂OH, or C₁-C₆ haloalkyl;
R₈ is —H or C₁-C₆ alkyl;
each R₉ is independently —H, halogen, —CN, C₁-C₆ alkyl, C₁-C₆ haloalkyl, C₁-C₆ alkoxy, or C₃-C₅ cycloalkyl;
each R₁₀ is independently —H, —CN, halogen, C₁-C₆ haloalkyl, C₁-C₆ alkoxy, C₁-C₆ haloalkoxy, —SO₂R₁₁, —SONR₁₁R₁₁, —CO₂H, —CO₂C₁-C₃ alkyl, —CONR₁₁R₁₂, —NR₁₁R₁₁, —NR₁₁—CO₂R₁₁, —NR₁₁—C(O)R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, an optionally substituted heteroaryl selected from pyrazole, isoxazole, isothiazole, imidazole, oxazole, thiazole, or pyridine, or a group of the formula.

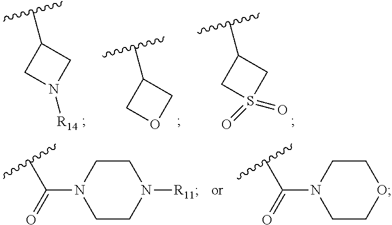

wherein the optionally substituted C₁-C₆ alkyl, C₂-C₆ alkenyl, or C₂-C₆ alkynyl is each optionally substituted with a —CN, —OH (e.g., where R₁₀ is C₁-C₆ hydroxyalkyl such as —CH₂—CH₂—OH and optionally where R₁₀ comprises a secondary —OH such as —CH₂—CH(CH₃)—OH or a tertiary —OH such as —CH₂—C(CH₃)₂—OH), oxetanyl, C₁-C₃ alkoxy, optionally substituted C₃-C₅ cycloalkyl, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN;
each R₁₁ is independently —H, C₁-C₃ alkyl, C₃-C₇ cycloalkyl, or C₁-C₃ haloalkyl;
each R₁₂ is independently —H, optionally substituted C₁-C₃ alkyl, C₃-C₆ cycloalkyl, —SO₂C₁-C₃ alkyl, —SO₂C₁-C₃ haloalkyl, —SO₂NR₁₁R₁₁, —NR₁₁R₁₁, —OR₁₁, —O—CH₂—CH(OH)—CH₂OH, —CN, oxetane, tetrahydrofuran, an aryl, a 5-member heteroaryl optionally substituted with a methyl, a 6-member heteroaryl, or a group of the formula

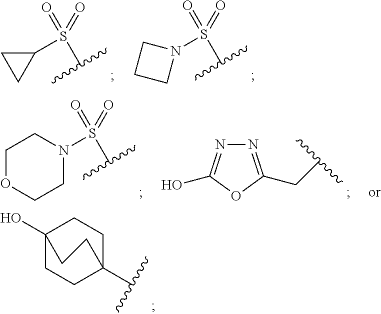

wherein the optionally substituted C₁-C₃ alkyl is optionally substituted with —OH, a C₃-C₆ cycloalkyl, oxetane, tetrahydrofuran, an aryl, a 5-member heteroaryl, a 6-member heteroaryl or an indole;
R₁₃ is —NR₁₁R₁₁, —OR₁₁, —SO₂C₁-C₃ alkyl, or a ring selected from oxetane, tetrahydrofuran or oxadiazole wherein the ring is optionally substituted with —NR₁₁R₁₁, or —OR₁₁;
R₁₄ is —H, optionally substituted C₁-C₃ alkyl, —SO₂C₁-C₃ alkyl, an aryl, a 5-member heteroaryl, or a 6-member heteroaryl; wherein the optionally substituted C₁-C₃ alkyl is optionally substituted with an aryl, a 5-member heteroaryl or a 6-member heteroaryl;
R₁₅ is an optionally substituted aryl, or an optionally substituted 6-member heteroaryl; wherein the optionally substituted aryl, or the optionally substituted 6-member heteroaryl is each optionally substituted with one to three substituents each independently selected from halogen, C₁-C₃ alkyl, or C₁-C₃ haloalkyl;
R₁₆ is H, C₁-C₃ alkyl, —NH₂, phenyl, or pyridine, and
n is 0, 1 or 2.

In one aspect, the present invention relates to compounds of Formula (I):

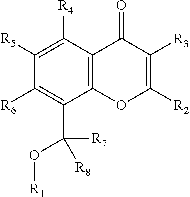

or pharmaceutically acceptable salt thereof, wherein:
R₁ is an optionally substituted bicyclic ring selected from isobenzofuranone, benzofuranone, isoindolinone, indolinone, quinazolinone, 3,4-dihydro-2H-isoquinolin-1-one, 2H-isoquinolin-1-one, or benzothiazolone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
R₁ is a group of the formula:

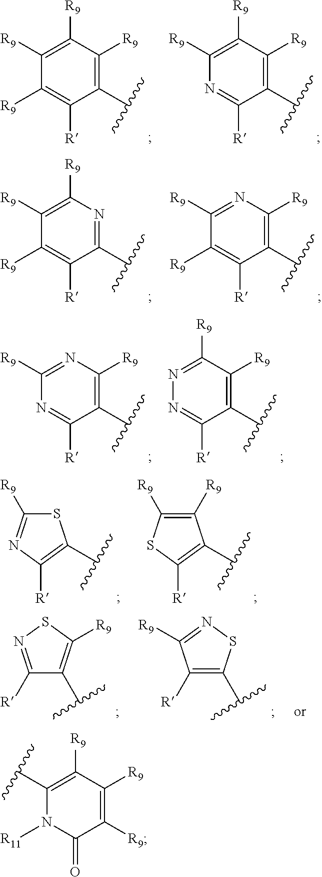

R′ is hydrogen, halogen, C₁-C₃ alkyl, C₁-C₃ haloalkyl, C₁-C₃ alkoxy, C₁-C₃ haloalkoxy, —OH, —CH(OH)—CH₂OH, —CO—CH₂OH, C₃-C₆ cycloalkyl, —NO₂, —N(R₁₁)—CO₂C₁-C₃ alkyl, —N(R₁₁)—SO₂C₁-C₃ alkyl, —N(R₁₁)—SO₂R₁₅, —SO₂C₁-C₃ alkyl, —SO₂NR₁₁R₁₁, —SO₂N(R₁₁)—CO—C₁-C₃ alkyl, —SO₂N(R₁₁)—CN, —C(═N—OH)—NH₂, —CN, —CONR₁₁R₁₂, —CON(R₁₁)—(CH₂)ₙ—R₁₃, —CO—SR₁₂, or a group of the formula:

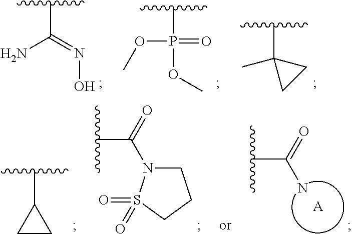

wherein ring A is pyrrolidine optionally substituted with a —CN; or
R′ is selected from oxetane, pyrrolidine, tetrahydrofuran, morpholine, piperidine, piperazine, pyrrole, furan, thiophene, pyrazole, imidazole, isoxazole, oxazole, isothiazole, thiazole, triazole, oxadiazole, thiadiazole, tetrazole, phenyl, pyridine, pyridazine, pyrimidine, pyrazine, or triazine; each of which is optionally substituted with one to three substituents independently selected from oxo, —OH, —NR₁₁R₁₁, —N(R₁₁)—CO—R₁₁, —N(R₁₁)—CN, —OR₁₁, —CN, halogen, C₁-C₆ haloalkyl or C₁-C₆ alkyl optionally substituted with an aryl, a 5-member heteroaryl or a 6-member heteroaryl; or
R′ is a group of the formula:

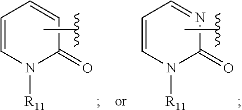

R₂ is a group of the formula:

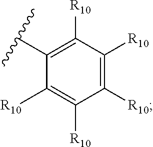

or
R₂ is a group of the formula:

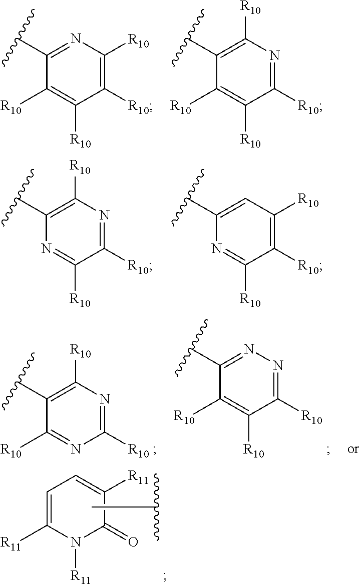

or
R₂ is an optionally substituted 5-member ring heteroaryl selected from pyrrole, furan, thiophene, pyrazole, isoxazole, isothiazole, imidazole, oxazole, thiazole, triazole, tetrazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —OH, —NR₁₁R₁₁, —NR₁₁CO₂R₁₁, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH (e.g., C₁-C₆ hydroxyalkyl such as —CH₂—CH₂—OH and optionally comprising a secondary —OH such as —CH₂—CH(CH₃)—OH or a tertiary —OH such as —CH₂—C(CH₃)₂—OH), halogen, oxetanyl, 2-oxabicyclo[2.1.1]hexane, C₁-C₃ alkoxy, an optionally substituted C₃-C₅ cycloalkyl, —CONR₁₁R₁₁, or —S(O)₂CH₃; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is an optionally substituted bicyclic ring selected from 1,3-benzodioxole, 2,3-dihydro-1,4-benzodioxine, quinoxaline, quinazoline, indole, indazole, isoindazole, benzimidazole, benzotriazole, benzothiazole, benzoxazole, benzotriazole, benzofuran, benzofurazan, pyridofurazan, quinoline, 1,5-naphthyridine, isoindolin-1-one, indolin-2-one, benzomorpholine, benzo[d]oxazol-2(3H)-one, imidazo[1,2-a]pyridine, 1,3-dihydro-2H-pyrrolo[2,3-b]pyridin-2-one, [1,3]dioxolo[4,5-b]pyridine, 6,7-dihydro-4H-pyrazolo[5,1-c][1,4]oxazine, 1,2,3,4-tetrahydroisoquinoline, 4,5,6,7-tetrahydropyrazolo[1,5-a]pyrazine, pyrazolo[4,3-b]pyridine, pyrazolo[3,4-b]pyridine, pyrazolo[3,4-c]pyridine, pyrazolo[1,5-a]pyrimidine, oxazolo[4,5-b]pyridine, oxazolo[5,4-b]pyridine, thiazolo[5,4-b]pyridine, 2,1,3-benzothiadiazole, imidazo[1,2-a]pyrimidine, or 2,3-dihydro-[1,4]dioxino[2,3-b]pyridine, 6,7-dihydro-5H-cyclopent[b]pyridine, 5,6-dihydro-4H-pyrrolo[1,2-b]pyrazole, 3,4-dihydro-2(1H)quinolinone, 2H-1,4-benzoxazin-3(4H)-one, 2-hydroxyquinoline, 3,4-dihydroisoquinolin-1,(2H)-one, 1-hydroxyisoquinoline, 1,4-dihydro-3(2H)-isoquinolinone, 3H-imidazo[4,5-b]pyridine, 4,5-dihydro-7H-pyrazolo[1,5-c]1,3]oxazine, 6,7-dihydro-5H-pyrazolo[5,1-b][1,3]oxazine, furo[3,2-c]pyridine, furo[3,2-b]pyridine, 2,3-dihydropyrazolo[5,1-b]oxazole, 5H,6H,8H-imidazo[2,1-c][1,4]oxazine, pyrazolo[1,5-b]pyridazine, imidazo[1,2-b]pyridazine, 2,4-dihydro-1H-benzo[d][1,3]oxazine, 5-oxaspiro[2,3]hexane, imidazo[1,5-a]pyridine, pyrazolo[1,5-a]pyridine; or an optionally substituted bicyclic heteroaryl of 8 to 10 ring atoms containing 1, 2, 3, 4, or 5 ring heteroatoms independently selected from N, O, or S; wherein the optionally substituted bicyclic ring is optionally substituted with one to three substituents each independently selected from halogen and C₁-C₆ alkyl; or wherein the optionally substituted bicyclic ring or the optionally substituted bicyclic heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —NR₁₁R₁₁, —NR₁₁CO₂R₁₁, —NR₁₁C(O)R₁₁, —OH, oxetanyl, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH (e.g., C₁-C₆ hydroxyalkyl such as —CH₂—CH₂—OH and optionally comprising a secondary —OH such as —CH₂—CH(CH₃)—OH or a tertiary —OH such as —CH₂—C(CH₃)₂—OH), oxetanyl, C₁-C₃ alkoxy, —CONR₁₁R₁₁, phenyl, or an optionally substituted C₃-C₅ cycloalkyl; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is a cyclopropyl, cyclobutyl, cyclopentyl, or cyclohexyl, each optionally substituted with one to three R₁₀ substituents, or R₂ is a group of the formula:

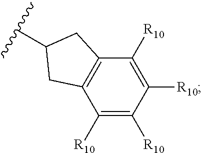

R₃ is —H, halogen, —CN, —N(H)(C₁-C₃ alkyl), —N(C₁-C₃ alkyl)₂, —N(H)(CH₂CH₂CO₂H), —CO—C₁-C₃ alkyl, C₁-C₆ alkyl, C₁-C₆ haloalkyl, C₁-C₆ hydroxyalkyl, C₃-C₅ cycloalkyl, an optionally substituted heterocycle of 3 to 5 ring atoms containing 1, 2, or 3 ring heteroatoms independently selected from N, O, or S, or an optionally substituted heteroaryl of 5 or 6 ring atoms containing 1, 2, or 3 ring heteroatoms independently selected from N, O, or S; wherein the optionally substituted heterocycle or heteroaryl is each optionally substituted with one to three substituents each independently selected from halogen, C₁-C₃ alkyl, or C₁-C₃ haloalkyl;
each of R₄, R₅ and R₆ is independently —H, halogen, C₁-C₆ alkyl or C₁-C₆ haloalkyl;
R₇ is —CN, C₁-C₆ alkyl or C₁-C₆ haloalkyl;
R₈ is —H or C₁-C₆ alkyl;
each R₉ is independently —H, halogen, —CN, C₁-C₆ alkyl, C₁-C₆ haloalkyl, C₁-C₆ alkoxy, or C₃-C₅ cycloalkyl;
each R₁₀ is independently —H, —CN, halogen, C₁-C₆ haloalkyl, C₁-C₆ alkoxy, C₁-C₆ haloalkoxy, —SO₂R₁₁, —SONR₁₁R₁₁, —CO₂H, —CO₂C₁-C₃ alkyl, —CONR₁₁R₁₂, —NR₁₁R₁₁, —NR₁₁—CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, an optionally substituted heteroaryl selected from pyrazole, isoxazole, isothiazole, imidazole, oxazole, thiazole, or pyridine, or a group of the formula.

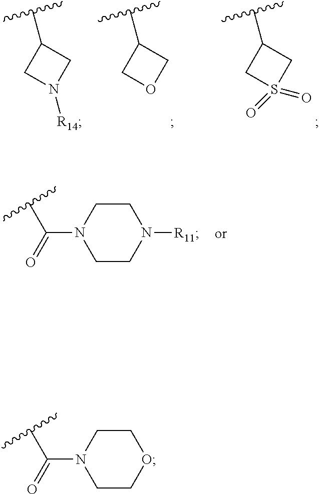

wherein the optionally substituted C₁-C₆ alkyl, C₂-C₆ alkenyl, or C₂-C₆ alkynyl is each optionally substituted with a —CN, —OH (e.g., where R₁₀ is C₁-C₆ hydroxyalkyl such as —CH₂—CH₂—OH and optionally comprises a secondary —OH such as —CH₂—CH(CH₃)—OH or a tertiary —OH such as —CH₂—C(CH₃)₂—OH), oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN;
each R₁ is independently —H or C₁-C₃ alkyl;
each R₁₂ is independently —H, optionally substituted C₁-C₃ alkyl, C₃-C₆ cycloalkyl, —SO₂C₁-C₃ alkyl, —SO₂C₁-C₃ haloalkyl, —SO₂NR₁₁R₁₁, —NR₁₁R₁₁, —OR₁₁, —O—CH₂—CH(OH)—CH₂OH, —CN, oxetane, tetrahydrofuran, an aryl, a 5-member heteroaryl optionally substituted with a methyl, a 6-member heteroaryl, or a group of the formula

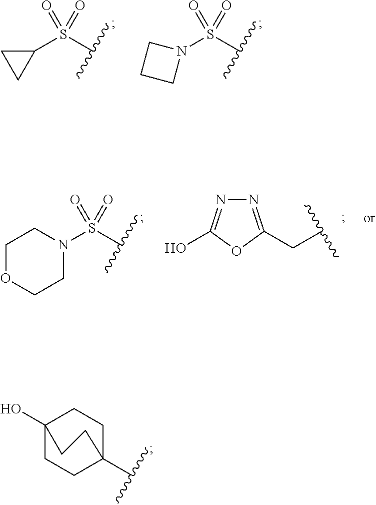 [SMILES: *S(=O)(=O)C1CC1 ; *S(=O)(=O)N1CCC1 ; *S(=O)(=O)N1CCOCC1 ; *Cc1nnc(O)o1 ; *C12CCC(O)(CC1)CC2]

wherein the optionally substituted C₁-C₃ alkyl is optionally substituted with —OH, a C₃-C₆ cycloalkyl, oxetane, tetrahydrofuran, an aryl, a 5-member heteroaryl, a 6-member heteroaryl or an indole;
R₁₃ is —NR₁₁R₁₁, —OR₁₁, —SO₂C₁-C₃ alkyl, or a ring selected from oxetane, tetrahydrofuran or oxadiazole wherein the ring is optionally substituted with —NR₁₁R₁₁, or —OR₁₁;
R₁₄ is —H, optionally substituted C₁-C₃ alkyl, —SO₂C₁-C₃ alkyl, an aryl, a 5-member heteroaryl, or a 6-member heteroaryl; wherein the optionally substituted C₁-C₃ alkyl is optionally substituted with an aryl, a 5-member heteroaryl or a 6-member heteroaryl; R₁₅ is an optionally substituted aryl, or an optionally substituted 6-member heteroaryl; wherein the optionally substituted aryl, or the optionally substituted 6-member heteroaryl is each optionally substituted with one to three substituents each independently selected from halogen, C₁-C₃ alkyl, or C₁-C₃ haloalkyl; and
n is 0, 1 or 2.

In one aspect, the compounds of Formula (I) may include an asymmetrical carbon atom at the position designated with an asterisk (*)

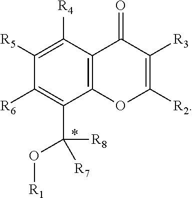

In one aspect, R₇ is methyl and R₈ is hydrogen and the bond at the * position is as represented as

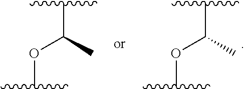

In a further aspect, compounds of Formula (I), wherein R₈ is —H, have Formula (II), or pharmaceutically acceptable salts thereof:

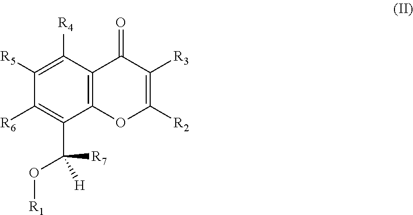

wherein R₁, R₂, R₃, R₄, R₅, R₆, and R₇, are as defined in the Summary for Formula (I).

In a further aspect, compounds of Formula (I) or (II) have Formula (III), or pharmaceutically acceptable salts thereof:

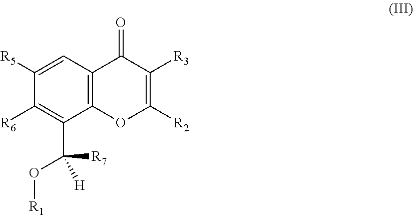

wherein R₁, R₂, R₃, R₅, R₆, and R₇ are as defined in the Summary for Formula (I) above.

In a further aspect, compounds of Formula (I), (II), or (III) have Formula (IV), or pharmaceutically acceptable salts thereof:

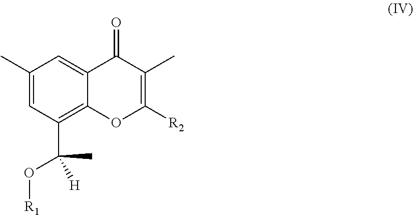

wherein R₁ and R₂ are as defined in the Summary for Formula (I) above.

In one aspect, the disclosed compounds include an R′ group which is not —COOH.

In one aspect, the disclosed compounds include an R₂ group which is bound to the chromenone core of the compound via a ring carbon atom of R₂, otherwise referred to as a “C-linked R₂ group.” In one aspect, the disclosed compounds include an R₂ group which is an aryl group bound to the chromenone core via a ring carbon atom of R₂. In one aspect, the disclosed compounds include an R₂ group which is a heteroaryl group bound to the chromenone core via a ring carbon atom of R₂.

In one aspect, the disclosed compounds include an R₂ group which is a bicyclic heteroaryl group bound to the chromenone core via a ring carbon atom of R₂. In one aspect, the disclosed compounds include an R₂ group which is a cycloalkyl group bound to the compound chromenone core via a ring carbon atom of R₂.

In one aspect, the disclosed compounds may have one or more activities as disclosed herein.

In one aspect, the disclosed compounds may inhibit the activity of PI3K-Alpha kinase and may be characterized as having (PIK3CA) inhibitory activity. In one aspect, the disclosed compounds may inhibit the activity of wild-type PIK3CA. In one aspect, the disclosed compounds may inhibit the activity of a mutant PIK3CA having one or more amino acid substitutions, deletions, or insertions relative to the amino acid sequence of wild-type PIK3CA. In one aspect, the disclosed compounds may selectively inhibit the activity of a mutant PI3KCA relative to wild-type PI3KCA.

In one aspect, the disclosed compounds may exhibit metabolic stability. In one aspect, the disclosed compounds may exhibit metabolic stability as measured in one or more in vitro clearance assays as disclosed herein.

In one aspect, the disclosed compounds do not induce expression of a cytochrome P450 enzyme in comparison to a control or vehicle in an in vitro assay, or the compounds induce expression of a cytochrome P450 enzyme at a relatively low level in comparison to a control or vehicle in an in vitro assay.

In one aspect, the disclosed compounds do not cause adverse events (AEs) such as hyperglycemia, hyperinsulinemia, and diarrhea when the compounds are administered at an effective dose for inhibiting the activity of PI3Kα and treating a disease or disorder associated with PI3Kα activity.

In one aspect, the disclosed compounds do not exhibit substantial inhibitory activity against kinases other than PI3Kα.

In another aspect, the present invention provides a pharmaceutical composition comprising a compound of Formula (I), (II), (III), or (IV), or a pharmaceutically acceptable salt thereof, and a pharmaceutically acceptable diluent, or carrier.

In another aspect, the present invention provides a method of modulating PI3K (e.g., PI3Kα(H1047R) or PI3Kα(E545K)) activity (e.g., in vitro, or in vivo), comprising contacting a cell with a therapeutically effective amount of a compound of Formula (I), (II), (III), or (IV), or a pharmaceutically acceptable salt thereof.

In some aspects, the present invention provides a method of treating, or preventing a disease, or disorder disclosed herein in a subject in need thereof, comprising administering to the subject a therapeutically effective amount of a compound of Formula (I), (II), (III), or (IV), or a pharmaceutically acceptable salt thereof. In some aspects, the compound of Formula (I), (II), (III), or (IV) is for first-line administration. In some aspects, the compound of Formula (I), (II), (III), or (IV) is for second-line administration. In some aspects, the compound of Formula (I), (II), (III), or (IV) is for third-line administration.

In some aspects, the present invention provides a method of treating, or preventing a disease, or disorder disclosed herein in a subject in need thereof, comprising administering to the subject a therapeutically effective amount of a pharmaceutical composition of a compound of Formula (I), (II), (III), or (IV), or a pharmaceutically acceptable salt thereof. In some aspects, the pharmaceutical composition of a compound of Formula (I), (II), (III), or (IV) is for first-line administration. In some aspects, the pharmaceutical composition of a compound of Formula (I), (II), (III), or (IV) is for second-line administration. In some aspects, the pharmaceutical composition of a compound of Formula (I), (II), (III), or (IV) is for third-line administration.

In some aspects, the present invention provides a method of treating a disease, or disorder disclosed herein in a subject in need thereof, comprising administering to the subject a therapeutically effective amount of a compound of Formula (I), (II), (III), or (IV), or a pharmaceutically acceptable salt thereof. In some aspects, the compound of Formula (I), (II), (III), or (IV) is administered as a first-line therapy. In some aspects, the compound of Formula (I), (II), (III), or (IV) is administered as a second-line therapy. In some aspects, the compound of Formula (I), (II), (III), or (IV) is administered as a third-line therapy.

In another aspect, the present invention provides a compound of Formula (I), (II), (III), or (IV), or a pharmaceutically acceptable salt thereof for use in therapy. In some aspects, the compound of Formula (I), (II), (III), or (IV) is used in first-line therapy. In some aspects, the compound of Formula (I), (II), (III), or (IV) is used in second-line therapy. In some aspects, the compound of Formula (I), (II), (III), or (IV) is used in third-line therapy.

In another aspect, the present invention provides a compound of Formula (I), (II), (III), or (IV), or a pharmaceutically acceptable salt thereof for use in modulating PI3K (e.g., PI3Kα(H1047R) or PI3Kα(E545K)) activity (e.g., in vitro, or in vivo).

In another aspect, the present invention provides a compound of Formula (I), (II), (III), or (IV), or a pharmaceutically acceptable salt thereof, for use in selective inhibition for mutant PI3Kα over wild-type PI3Kα.

In another aspect, the present invention provides a compound of Formula (I), (II), (III), or (IV), or a pharmaceutically acceptable salt thereof, for use in selective inhibition of PI3Kα(H1047R mutant) over wild-type PI3Kα.

In another aspect, the present invention provides a compound of Formula (I), (II), (III), or (IV), or a pharmaceutically acceptable salt thereof, for use in selective inhibition of PI3Kα(E545K mutant) over wild-type PI3Kα.

In another aspect, the present invention provides a compound of Formula (I), (II), (III), or (IV), or a pharmaceutically acceptable salt thereof, for use in treating, or preventing a disease, or disorder disclosed herein. In some aspects, the compound of Formula (I), (II), (III), or (IV) is for first-line administration. In some aspects, the compound of Formula (I), (II), (III), or (IV) is for second-line administration. In some aspects, the compound of Formula (I), (II), (III), or (IV) is for third-line administration.

In another aspect, the present invention provides a compound of Formula (I), (II), (III), or (IV), or a pharmaceutically acceptable salt thereof, for use in treating a disease, or disorder disclosed herein. In some aspects, the compound of Formula (I), (II), (III), or (IV) is used for first-line treatment. In some aspects, the compound of Formula (I), (II), (III), or (IV) is used for second-line treatment. In some aspects, the compound of Formula (I), (II), (III), or (IV) is used for third-line treatment.

In another aspect, the present invention provides use of a compound of Formula (I), (II), (III), or (IV), or a pharmaceutically acceptable salt thereof, in the manufacture of a medicament for modulating PI3K (e.g., PI3Kα(H1047R) or PI3Kα(E545K)) activity (e.g., in vitro, or in vivo).

In another aspect, the present invention provides use of a compound of Formula (I), (II), (III), or (IV), or a pharmaceutically acceptable salt thereof, in the manufacture of a medicament for treating, or preventing a disease, or disorder disclosed herein. In some aspects, the medicament is used for first-line administration. In some aspect, the medicament is used for second-line administration. In some aspect, the medicament is used for third-line administration.

In another aspect, the present invention provides use of a compound of Formula (I), (II), (III), or (IV), or a pharmaceutically acceptable salt thereof, in the manufacture of a medicament for treating a disease, or disorder disclosed herein. In some aspects, the medicament is used for first-line treatment. In some aspect, the medicament is used for second-line treatment. In some aspect, the medicament is used for third-line treatment.

In another aspect, the present invention provides a method of preparing a compound of Formula (I), (II), (III), or (IV), or a pharmaceutically acceptable salt thereof.

In another aspect, the present invention provides a method of preparing a compound, of Formula (I), (II), (III), or (IV), or a pharmaceutically acceptable salt thereof, comprising one, or more steps described herein.

In another aspect, the present invention provides a compound obtainable by, or obtained by, a method for preparing a compound as described herein.

In another aspect, the present invention provides an intermediate as described herein, being suitable for use in a method for preparing a compound as described herein (e.g., the intermediate is selected from the intermediates described in the Examples).

Other features, and advantages of the invention will be apparent from the following detailed description, and claims.

### DETAILED DESCRIPTION

The present invention provides methods of treating, preventing, or ameliorating a disease, or disorder, (or uses in the treatment, prevention, or amelioration of a disease, or disorder), in which PI3K plays a role by administering to a patient in need thereof a therapeutically effective amount of a PI3K inhibitor of the present invention. The methods (or uses) of the present invention can be used in the treatment of a variety of PI3K-dependent diseases, and disorders.

In some embodiments, the disease, or disorder is a cancer (e.g., breast cancer, brain cancers, prostate cancer, endometrial cancer, gastric cancer, leukemia, lymphoma, sarcoma, colorectal cancer, lung cancer, ovarian cancer, skin cancer, or head and neck cancer). In some embodiments, the disease, or disorder associated with PI3K includes, but is not limited to, CLOVES syndrome, PROS, endometrial cancer, breast cancer, esophageal squamous-cell cancer, cervical squamous-cell carcinoma, cervical adenocarcinoma, colorectal adenocarcinoma, bladder urothelial carcinoma, glioblastoma, ovarian cancer, non-small-cell lung cancer, esophagogastric cancer, nerve-sheath tumor, head and neck squamous-cell carcinoma, melanoma, esophagogastric adenocarcinoma, soft-tissue sarcoma, prostate cancer, fibrolamellar carcinoma, hepatocellular carcinoma, diffuse glioma, colorectal cancer, pancreatic cancer, cholangiocarcinoma, B-cell lymphoma, mesothelioma, adrenocortical carcinoma, renal non-clear-cell carcinoma, renal clear-cell carcinoma, germ-cell carcinoma, thymic tumor, pheochromocytoma, miscellaneous neuroepithelial tumor, thyroid cancer, leukemia, and encapsulated glioma.

The details of the invention are set forth in the accompanying description below. Although methods, and materials similar, or equivalent to those described herein can be used in the practice, or testing of the present disclosure, illustrative methods, and materials are now described. Other features, objects, and advantages of the invention will be apparent from the description, and from the claims. In the specification, and the appended claims, the singular forms also include the plural unless the context clearly dictates otherwise. Unless defined otherwise, all technical, and scientific terms used herein have the same meaning as commonly understood by one of ordinary skill in the art to which this invention belongs. All patents, and publications cited in this specification are incorporated herein by reference in their entireties.

### Definitions

The articles “a”, and “an” refer to one, or more than one (i.e., to at least one) of the grammatical object of the article. By way of example, “an element” means one element, or more than one element.

The term “and/or” means either “and”, or “or” unless indicated otherwise.

The term “administer”, “administering”, or “administration” refers to either directly administering a disclosed compound, or pharmaceutically acceptable salt of the disclosed compound, or a composition to a subject.

The term “alkenyl” refers to a straight, or branched chain unsaturated hydrocarbon containing 2-12 carbon atoms. The “alkenyl” group contains at least one double bond in the chain. The double bond of an alkenyl group can be unconjugated, or conjugated to another unsaturated group.

Examples of alkenyl groups include ethenyl, propenyl, n-butenyl, iso-butenyl, pentenyl, or hexenyl.

The term “alkoxy” refers to a straight, or branched chain saturated hydrocarbon containing 1-12 carbon atoms containing a terminal “O” in the chain, i.e., —O(alkyl). Examples of alkoxy groups include without limitation, methoxy, ethoxy, propoxy, butoxy, t-butoxy, or pentoxy groups.

The term “alkyl” refers to a straight, or branched chain saturated hydrocarbon containing 1-12 carbon atoms, preferably 1-6 carbon atoms. Examples of a C₁-C₆ alkyl group include, but are not limited to, methyl, ethyl, propyl, butyl, pentyl, hexyl, isopropyl, isobutyl, sec-butyl, tert-butyl, isopentyl, neopentyl, and isohexyl.

The term “alkynyl” refers to a straight, or branched chain unsaturated hydrocarbon containing 2-12 carbon atoms. The “alkynyl” group contains at least one triple bond in the chain. Examples of alkynyl groups include ethynyl, propargyl, n-butynyl, iso-butynyl, pentynyl, or hexynyl.

It is understood that for example the terms “alkenyl”, “alkoxy”, “alkyl”, “alkynyl”, “haloalkyl”, “haloalkoxy” and “cycloalkyl” can be further defined by the numbers of carbons they contain, such as for example the term “C₁-C₆ alkoxy” refers to an alkoxy group as defined above containing 1-6 carbon atoms.

The term “hydroxyalkyl” means an alkyl group, which may be straight chain or branched, having one or more hydrogen atoms substituted with a hydroxyl. For example “hydroxyalkyl” includes hydroxyethyl, 2-hydroxylpropyl, and 1,2-dihydroxybutyl. A hydroxyl group of a hydroxyalkyl may be a primary hydroxyl group where the hydroxyl group is covalently bound to a carbon atom which is bound to two hydrogen atoms as in (—CH₂)₁₋₆—OH. A hydroxyl group of a hydroxyalkyl may be a secondary hydroxyl group where the hydroxyl group is covalently bound to a carbon atom which is bound to methyl group and a hydrogen atom as in (—CH₂)₁₋₆—CH(CH₃)—OH. A hydroxyl group of a hydroxyalkyl may be a tertiary hydroxyl group where the hydroxyl group is covalently bound to a carbon atom which is bound to two methyl group as in (—CH₂)₁₋₆—C(CH₃)₂—OH. An alkyl group comprising a primary hydroxyl, secondary hydroxyl, and tertiary hydroxyl may be represented as

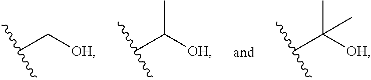

respectively.

The term “aromatic” means a planar ring having 4n+2 electrons in a conjugated system. As used herein, “conjugated system” means a system of connected p-orbitals with delocalized electrons, and the system may include lone electron pairs.

The term “aryl” unless otherwise specifically defined refers to cyclic, aromatic hydrocarbon groups that have 1 to 3 aromatic rings, including monocyclic, or bicyclic groups such as phenyl, biphenyl, or naphthyl. Where containing two aromatic rings (bicyclic, etc.), the aromatic rings of the aryl group may be joined at a single point (e.g., biphenyl), or fused (e.g., naphthyl). Furthermore, when containing two fused rings the aryl groups herein defined may have one, or more saturated, or partially unsaturated ring fused with a fully unsaturated aromatic ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, and tetrahydrobenzoannulenyl.

The term “carrier” encompasses carriers, excipients, and diluents, and means a material, composition, or vehicle, such as a liquid, or solid filler, diluent, excipient, solvent, or encapsulating material, involved in carrying, or transporting a pharmaceutical agent from one organ, or portion of the body, to another organ, or portion of the body of a subject.

The term “cyano” means a substituent having a carbon atom joined to a nitrogen atom by a triple bond, i.e., C≡N.

The term “cycloalkyl” means mono, or polycyclic saturated carbon rings containing 3-18 carbon atoms, preferably 3-10 carbon atoms. Examples of cycloalkyl groups include, without limitation, cyclopropyl, cyclobutyl, cyclopentyl, cyclohexyl, cycloheptanyl, cyclooctanyl, norbornyl, norbornenyl, bicyclo[2.2.2]octanyl, and bicyclo[2.2.2]octenyl.

The term “disorder” means, and is used interchangeably with, the terms disease, condition, or illness, unless otherwise indicated.

The term “haloalkoxy” refers to an alkoxy group, as defined herein, which is substituted with one, or more halogen. Examples of haloalkoxy groups include, but are not limited to, trifluoromethoxy, difluoromethoxy, pentafluoroethoxy, and trichloromethoxy.

The term “haloalkyl” refers to an alkyl group, as defined herein, which is substituted with one, or more halogen. Examples of haloalkyl groups include, but are not limited to, trifluoromethyl, difluoromethyl, pentafluoroethyl, and trichloromethyl.

The term “halogen” or “halo” refers to fluorine, chlorine, bromine, or iodine.

The term “heteroaryl” unless otherwise specifically defined means a monovalent monocyclic, or a polycyclic aromatic radical of 5 to 24 ring atoms, preferably 5 to 10 ring atoms, containing one, or more ring heteroatoms selected from N, O, S, P, or B, preferably 1, 2, 3, or 4 ring heteroatoms selected from N, O, or S, the remaining ring atoms being C. A polycyclic aromatic radical includes two, or more fused rings, and may further include two, or more spiro-fused rings, e.g., bicyclic, tricyclic, tetracyclic, and the like. Unless otherwise specifically defined, “fused” means two rings sharing two ring atoms. Unless otherwise specifically defined, “spiro-fused” means two rings sharing one ring atom. Heteroaryl as herein defined also means a bicyclic heteroaromatic group wherein the heteroatom is selected from N, O, S, P, or B, preferably N, O, or S. Heteroaryl as herein defined also means a tricyclic heteroaromatic group containing one, or more ring heteroatoms selected from N, O, S, P, or B, preferably N, O, or S. Heteroaryl as herein defined also means a tetracyclic heteroaromatic group containing one, or more ring heteroatoms selected from N, O, S, P, or B, preferably N, O, or S. Examples of heteroaromatic groups include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuranyl, chromanyl, thiochromanyl, tetrahydroquinolinyl, dihydrobenzothiazinyl, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydro pyrrolo[1,2-a]pyrimidinyl, 3,4-dihydro-2H-1-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, and 3H-indolyl. Furthermore, when containing two, or more fused rings, the heteroaryl groups defined herein may have one, or more saturated, or partially unsaturated ring fused with one, or more fully unsaturated aromatic ring. In heteroaryl ring systems containing more than two fused rings, a saturated, or partially unsaturated ring may further be fused with a saturated, or partially unsaturated ring described herein. Furthermore, when containing three, or more fused rings, the heteroaryl groups defined herein may have one, or more saturated, or partially unsaturated ring spiro-fused. Any saturated, or partially unsaturated ring described herein is optionally substituted with one, or more oxo. Exemplary ring systems of these heteroaryl groups include, for example, indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuranyl, benzofuranonyl, oxindolyl, indolyl, 1,6-dihydro-7H-pyrazolo[3,4-c]pyridin-7-onyl, 7,8-dihydro-6H-pyrido[3,2-b]pyrrolizinyl, 8H-pyrido[3,2-b]pyrrolizinyl, 1,5,6,7-tetrahydrocyclopenta[b]pyrazolo[4,3-e]pyridinyl, 7,8-dihydro-6H-pyrido[3,2-b]pyrrolizinyl, pyrazolo[1,5-a]pyrimidin-7(4H)-onyl, 3,4-dihydropyrazino[1,2-a]indol-1(2H)-onyl, benzo[c][1,2]oxaborol-1(3H)-olyl, 6,6a,7,8-tetrahydro-9H-pyrido[2,3-b]pyrrolo[1,2-d][1,4]oxazin-9-onyl, and 6a′,7′-dihydro-6′H,9′H-spiro[cyclopropane-1,8′-pyrido[2,3-b]pyrrolo[1,2-d][1,4]oxazin]-9′-onyl.

The term “5-member heteroaryl” unless otherwise specifically defined means a monovalent monocyclic aromatic radical of 5 ring atoms, containing one, or more ring heteroatoms selected from N, O, S, P, or B, preferably 1, 2, 3, or 4 ring heteroatoms selected from N, O, or S, the remaining ring atoms being C. Exemplary 5-member heteroaryl groups include, but are not limited to, furyl, thiophenyl, pyrrolyl, pyrazolyl, imidazolyl, isoxazolyl, oxazolyl, oxadiazolyl, isothiazolyl, thiazolyl, thiadiazole, triazolyl and tetrazolyl.

The term “6-member heteroaryl” unless otherwise specifically defined means a monovalent monocyclic aromatic radical of 6 ring atoms, containing one, or more ring heteroatoms selected from N, O, S, P, or B, preferably 1, 2, 3, or 4 ring heteroatoms selected from N, O, or S, the remaining ring atoms being C. Exemplary 6-member heteroaryl groups include, but are not limited to, pyridyl, pyridazinyl, pyrimidinyl, pyrazinyl, or triazinyl.

The term “heterocyclyl”, “heterocycle”, or “heterocycloalkyl” means mono, or polycyclic rings containing 3-24 atoms, preferably 3-10 atoms, which include carbon, and one, or more heteroatoms selected from N, O, S, P, or B, preferably 1, 2, 3, or 4 heteroatoms selected from N, O, and S, and wherein the rings are not aromatic. Examples of heterocyclyl rings include, but are not limited to, oxetanyl, azetidinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, and homotropanyl.

The term “isomers” refers to compounds that have the same molecular formula but differ in the nature or sequence of bonding of their atoms or the arrangement of their atoms in space. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomers or as a mixture thereof. A mixture containing equal proportions of the enantiomers is called a “racemic mixture”.

The term “modulate”, “modulation”, or “modulating” refers to a biological activity of a compound, or substrate that inhibits and/or activates PI3K.

The term “patient”, or “subject” is a mammal, e.g., a human, mouse, rat, guinea pig, dog, cat, horse, cow, pig, or non-human primate, such as a monkey, chimpanzee, baboon, or rhesus. Preferably, the mammal is human.

The term “therapeutically effective amount” when used in connection with a compound refers to the amount or dose of the compound which upon single or multiple dose administration to the patient, provides the desired effect in the patient under diagnosis or treatment. An effective amount can be determined by one skilled in the art by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

The term “treating” with regard to a subject, includes restraining, slowing, stopping, or reversing the progression or severity of an existing symptom or disorder.

### Compounds of the Present Invention

In one aspect, the present invention provides compounds of Formula (I), or pharmaceutically acceptable salts thereof:

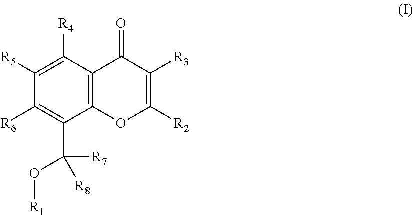

wherein R₁, R₂, R₃, R₄, R₅, R₆, R₇, and R₈, are as defined in the Summary for Formula (I).

In a compound of Formula (I), or pharmaceutically acceptable salts thereof, R₈ is —H.

In a further aspect, compounds of Formula (I), wherein R₈ is —H, have Formula (II), or pharmaceutically acceptable salts thereof:

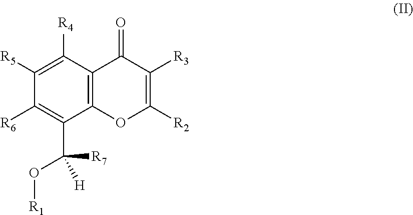

wherein R₁, R₂, R₃, R₄, R₅, R₆, and R₇, are as defined in the Summary for Formula (I).

In yet a further compound of Formula (I), or (II), or pharmaceutically acceptable salts thereof, R₄ is —H or halogen. Preferably R₄ is —H.

In a further aspect, compounds of Formula (I) or (II) have Formula (III), or pharmaceutically acceptable salts thereof:

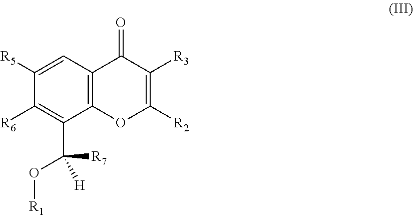

wherein R₁, R₂, R₃, R₅, R₆, and R₇ are as defined in the Summary for Formula (I) above.

In a further aspect, compounds of Formula (I), (II), or (III) have Formula (IV), or pharmaceutically acceptable salts thereof:

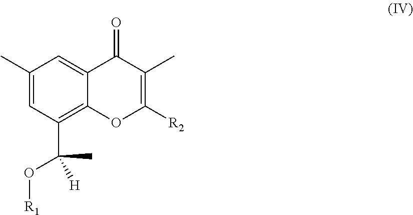

wherein R₁ and R₂ are as defined in the Summary for Formula (I) above.

In yet a further compound of Formula (I), (II), or (III), or pharmaceutically acceptable salts thereof, R₃ is —H, halogen, —CN, C₁-C₆ alkyl, C₁-C₆ haloalkyl, oxetane, oxazole, or isoxazole. Preferably R₃ is —H, —CN, C₁-C₃ alkyl, or C₁-C₃ haloalkyl. More preferably R₃ is —H, —CN, methyl, or trifluoromethyl. Most preferably R₃ is —H or methyl.

In yet a further compound of Formula (I), (II), or (III), or pharmaceutically acceptable salts thereof, R₅ is —H, halogen, C₁-C₃ alkyl or C₁-C₃ haloalkyl. Preferably R₅ is —H, halogen, methyl, or trifluoromethyl.

In yet a further compound of Formula (I), (II), or (III), or pharmaceutically acceptable salts thereof, R₆ is —H or halogen.

In yet a further compound of Formula (I), (II), or (III), or pharmaceutically acceptable salts thereof, R₇ is —CN, C₁-C₃ alkyl or C₁-C₃ haloalkyl. Preferably R₇ is —CN, methyl or trifluoromethyl.

More preferably R₇ is methyl.

In yet a further compound of Formula (I), (II), (III), or (IV), or pharmaceutically acceptable salts thereof, R₁ is an optionally substituted bicyclic ring selected from isobenzofuranone, benzofuranone, isoindolinone, indolinone, quinazolinone, or benzothiazolone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy.

In yet a further compound of Formula (I), (II), (III), or (IV), or pharmaceutically acceptable salts thereof, R₁ is a group of the formula

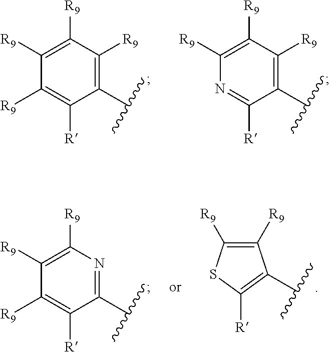

In yet a further compound of Formula (I), (II), (III), or (IV), or pharmaceutically acceptable salts thereof, R₁ is a group of the formula

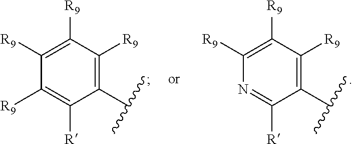

In yet a further compound of Formula (I), (II), (III), or (IV), or pharmaceutically acceptable salts thereof, each R₉ is independently —H, halogen, C₁-C₃ alkyl, or C₃-C₅ cycloalkyl, preferably each R₉ is independently —H, halogen, methyl, or cyclopropyl.

In yet a further compound of Formula (I), (II), (III), or (IV), or pharmaceutically acceptable salts thereof, each R₉ is independently —H, halogen, C₁-C₃ alkyl, or C₁-C₃ alkoxy.

In yet a further compound of Formula (I), (II), (III), or (IV), or pharmaceutically acceptable salts thereof, R′ is C₁-C₃ haloalkyl, C₁-C₃ alkoxy, —NO₂, —N(R)—CO₂C₁-C₃ alkyl, —N(R)—SO₂C₁-C₃ alkyl, —SO₂NR₁₁R₁₁, —SO₂N(R)—CO—C₁-C₃ alkyl, —C(═N—OH)—NH₂, —CN, —CONR₁₁R₁₂, —CON(R₁₁)—(CH₂)ₙ—R₁₃, or

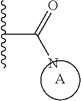

wherein ring A is pyrrolidine optionally substituted with a —CN.

In yet a further compound of Formula (I), (II), (III), or (IV), or pharmaceutically acceptable salts thereof, R₁ is selected from

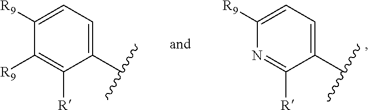

wherein R₉ is selected from hydrogen and halogen.

In yet a further compound of Formula (I), (II), (III), or (IV), or pharmaceutically acceptable salts thereof, R₁ is selected from

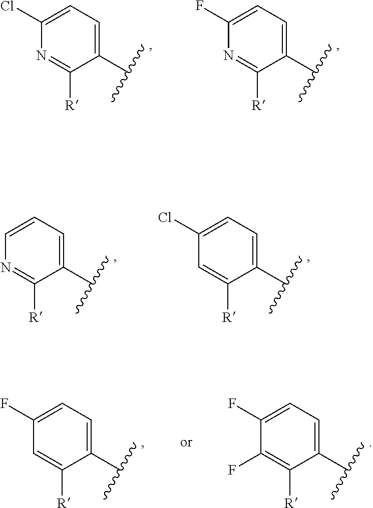

In yet a further compound of Formula (I), (II), (III), or (IV), or pharmaceutically acceptable salts thereof, R′ is not —COOH.

In yet a further compound of Formula (I), (II), (III), or (IV), or pharmaceutically acceptable salts thereof, R′ is C₁-C₃ haloalkyl, C₁-C₃ alkoxy, —NO₂, —N(R)—CO₂C₁-C₃ alkyl, —N(R)—SO₂C₁-C₃ alkyl, —SO₂NR₁₁R₁₁, —SO₂N(R)—CO—C₁-C₃ alkyl, —C(═N—OH)—NH₂, —CN, —CONR₁₁R₁₂, —CON(R₁₁)—(CH₂)ₙ—R₁₃, or

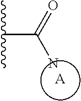

wherein ring A is pyrrolidine optionally substituted with a —CN.

In yet a further compound of Formula (I), (II), (III), or (IV), or pharmaceutically acceptable salts thereof, R′ is selected from oxetane, pyrrolidine, tetrahydrofuran, pyrrole, furan, thiophene, pyrazole, imidazole, isoxazole, oxazole, isothiazole, thiazole, triazole, oxadiazole, thiadiazole, tetrazole, pyridine, pyridazine, pyrimidine, pyrazine, or triazine; each of which is optionally substituted with one to three substituents independently selected from oxo, —NR₁₁R₁₁, —OR₁₁, —CN, C₁-C₆ haloalkyl or C₁-C₆ alkyl optionally substituted with phenyl.

In yet a further compound of Formula (I), (II), (III), or (IV), or pharmaceutically acceptable salts thereof, R′ is pyrrole, furan, thiophene, pyrazole, isoxazole, oxazole, isothiazole, thiazole, imidazole, triazole, oxadiazole, thiadiazole, tetrazole, pyridine, pyridazine, pyrimidine, pyrazine, or triazine; each of which is optionally substituted with one to three substituents independently selected from oxo, —NR₁₁R₁₁, —OR₁₁ or C₁-C₆ alkyl optionally substituted with aryl.

In yet a further compound of Formula (I), (II), (III), or (IV), or pharmaceutically acceptable salts thereof, R′ is hydrogen, halogen, C₁-C₃ alkyl, or C₃-C₆ cycloalkyl, preferably R′ is hydrogen, halogen, or methyl.

In yet a further compound of Formula (I), (II), (III), or (IV), or pharmaceutically acceptable salts thereof, R′ is hydrogen, halogen, C₁-C₃ alkyl, C₁-C₃ haloalkyl, C₁-C₃ hydroxyalkyl, C₁-C₃ alkoxy, C₁-C₃ haloalkoxy, —OH, —CH(OH)—CH₂OH, —CH(OH)C₁-C₃ haloalkyl, —CO—CH₂OH, C₃-C₆ cycloalkyl, —NO₂, —NR₁₁R₁₁, —N(R₁₁)—CO₂C₁-C₃ alkyl, —N(R)—SO₂C₁-C₃ alkyl, —N(R)—SO₂R₁₅, —SO₂C₁-C₃ alkyl, —SOC₁-C₃ alkyl, —SO₂NR₁₁R₁₁, —SO₂N(R₁₁)—CO—C₁-C₃ alkyl, —SO₂N(R₁₁)—CN, —SO₂N(R₁₁)(R₁₃)—C(═N—OH)—NH₂, —CN, —CONR₁₁R₁₂, —CON(R₁₁)—(CH₂)ₙ—R₁₃, —CO—SR₁₂, —CO—NHSO₂R₁₆, —COCH═SOR₁₁(R₁₁), or —COCH₂CN.

In yet a further compound of Formula (I), (II), (III), or (IV), or pharmaceutically acceptable salts thereof, R′ is not —COOH.

In yet a further compound of Formula (I), (II), (III), or (IV), or pharmaceutically acceptable salts thereof, R′ is —SO₂NR₁₁R₁₁, —SO₂N(R₁₁)(R₁₃), —C(O)NR₁₁R₁₂, —C(O)N(R)—(CH₂)ₙ—R₁₃, —C(O)—NHSO₂R₁₆, or oxadiazolinone (e.g., 1,2,4-oxadiazolin-5-one).

In yet a further compound of Formula (I), (II), (III), or (IV), or pharmaceutically acceptable salts thereof, R′ is —SO₂NR₁₁R₁₁.

In yet a further compound of Formula (I), (II), (III), or (IV), or pharmaceutically acceptable salts thereof, R′ is —SO₂N(R₁₁)(R₁₃).

In yet a further compound of Formula (I), (II), (III), or (IV), or pharmaceutically acceptable salts thereof, R′ is —C(O)NR₁₁R₁₂.

In yet a further compound of Formula (I), (II), (III), or (IV), or pharmaceutically acceptable salts thereof, R′ is —C(O)N(R₁₁)—(CH₂)ₙ—R₁₃.

In yet a further compound of Formula (I), (II), (III), or (IV), or pharmaceutically acceptable salts thereof, R′ is —C(O)—NHSO₂R₁₆.

In yet a further compound of Formula (I), (II), (III), or (IV), or pharmaceutically acceptable salts thereof, R′ is oxadiazolinone, preferably 1,2,4-oxadiazolin-5-one.

In yet a further compound of Formula (I), (II), (III), or (IV), or pharmaceutically acceptable salts thereof, R′ is hydrogen, C₁-C₃ alkyl, —CONH—SO₂C₁-C₃ alkyl, or a group of the formula

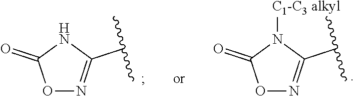

In yet a further compound of Formula (I), (II), (III), or (IV), or pharmaceutically acceptable salts thereof, R′ is hydrogen, methyl, —CONH—SO₂Me, or a group of the formula

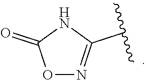 [SMILES: *c1noc(=O)[nH]1]

In yet a further compound of Formula (I), (II), (III), or (IV), or pharmaceutically acceptable salts thereof, R₁ is a group of the formula

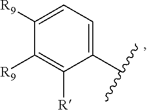

wherein each R₉ is independently —H, halogen, C₁-C₃ alkyl, or C₃-C₅ cycloalkyl, and R′ is hydrogen, halogen, C₁-C₃ alkyl, or C₃-C₆ cycloalkyl, preferably each R₉ is independently —H, halogen, methyl, or cyclopropyl and R′ is hydrogen, halogen, or C₁-C₃ alkyl.

In yet a further compound of Formula (I), (II), (III), or (IV), or pharmaceutically acceptable salts thereof, R₁ is a group of the formula

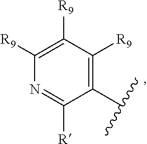

wherein each R₉ is independently —H, halogen, C₁-C₃ alkyl, or C₃-C₅ cycloalkyl, and R′ is hydrogen, halogen, C₁-C₃ alkyl, or C₃-C₆ cycloalkyl, preferably each R₉ is independently —H, halogen, methyl, or cyclopropyl and R′ is hydrogen, halogen, or C₁-C₃ alkyl.

In yet a further compound of Formula (I), (II), (III), or (IV), or pharmaceutically acceptable salts thereof, R₁ is a group of the formula

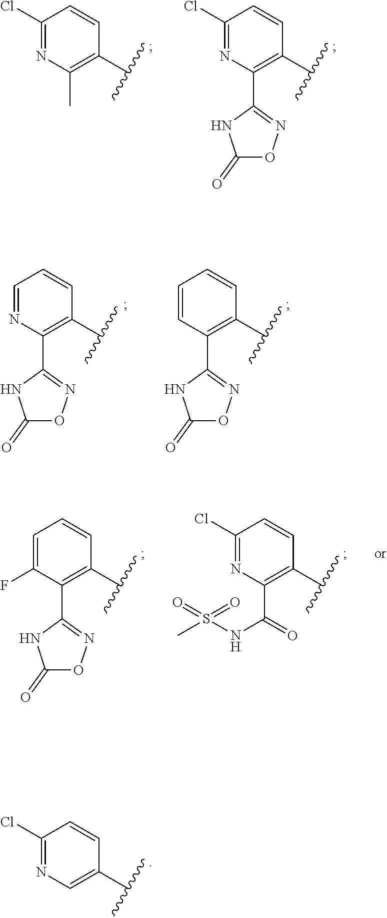

In yet a further compound of Formula (I), (II), (III), or (IV), or pharmaceutically acceptable salts thereof, R₂ is bound to the chromenone core of the compound via a ring carbon atom of R₂, otherwise referred to as a “C-linked R₂ group.” In one aspect, the disclosed compounds include an R₂ group which is an aryl group bound to the chromenone core via a ring carbon atom of R₂. In one aspect, the disclosed compounds include an R₂ group which is a heteroaryl group bound to the chromenone core via a ring carbon atom of R₂. In one aspect, the disclosed compounds include an R₂ group which is a bicyclic heteroaryl group bound to the chromenone core via a ring carbon atom of R₂. In one aspect, the disclosed compounds include an R₂ group which is a cycloalkyl group bound to the compound chromenone core via a ring carbon atom of R₂.

In yet a further compound of Formula (I), (II), (III), or (IV), or pharmaceutically acceptable salts thereof, R₂ is a group of the formula

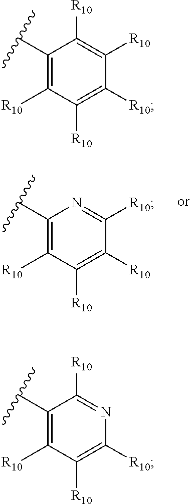

wherein each R₁₀ is independently —H, —CN, halogen, C₁-C₆ alkyl, C₁-C₆ haloalkyl, or —CO₂C₁-C₃ alkyl; or R₂ is an optionally substituted pyrazole or an optionally substituted indazole, each of which is optionally substituted with one to three substituents each independently selected from —CN, halogen, C₁-C₆ alkyl, C₁-C₆ haloalkyl, or —CO₂C₁-C₃ alkyl. Preferably R₂ is a group of the formula

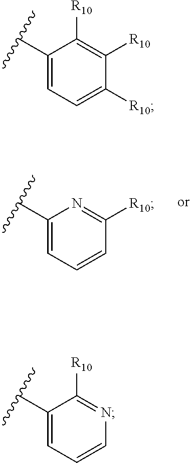

wherein each R₁₀ is independently —H, —CN, or halogen; or R₂ is an optionally substituted pyrazole or an optionally substituted indazole, each of which is optionally substituted with a C₁-C₃ alkyl.

In yet a further compound of Formula (I), (II), (III), or (IV), or pharmaceutically acceptable salts thereof, R₂ is a group of the formula:

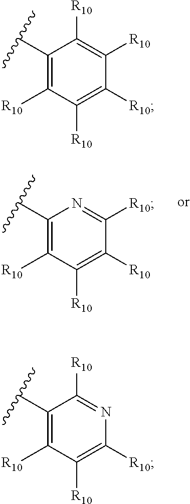

wherein each R₁₀ is independently —H, —CN, C₁-C₆ haloalkyl, —NR₁₁R₁₁, —CH₂—NR₁₁R₁₁, —CO₂C₁-C₃ alkyl or —CH₂—CO₂C₁-C₃ alkyl; or R₂ is an optionally substituted indazole, which is optionally substituted with one to three substituents each independently selected from —CN, halogen, C₁-C₆ alkyl, or C₁-C₆ haloalkyl.

In yet a further compound of Formula (I), (II), (III), or (IV), or pharmaceutically acceptable salts thereof, R₂ is a group of the formula

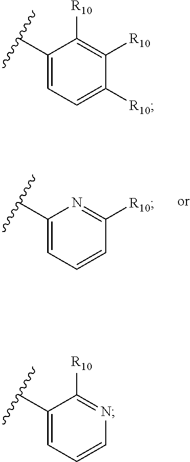

wherein each R₁₀ is independently —H, —CN, or C₁-C₃ haloalkyl; or R₂ is an optionally substituted indazole, which is optionally substituted with a C₁-C₃ alkyl.

In yet a further compound of Formula (I), (II), (III), or (IV), or pharmaceutically acceptable salts thereof, R₂ is a group of the formula

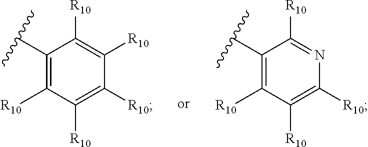

wherein each R₁₀ is independently —H, —CN, halogen, C₁-C₆ alkyl, or C₁-C₆ haloalkyl.

In yet a further compound of Formula (I), (II), (III), or (IV), or pharmaceutically acceptable salts thereof, R₂ is

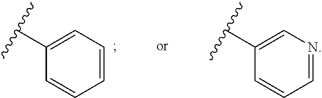 [SMILES: *c1ccccc1 ; ";" ; "or" ; *c1cccnc1]

In yet a further compound of Formula (I), (II), (III), or (IV), or pharmaceutically acceptable salts thereof, R₂ is a group of the formula:

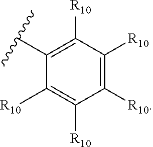

In yet a further compound of Formula (I), (II), (III), or (IV), or pharmaceutically acceptable salts thereof, R₂ is a group of the formula:

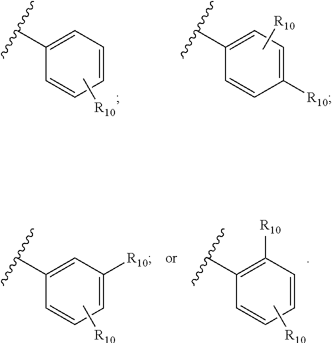

In yet a further compound of Formula (I), (II), (III), or (IV), or pharmaceutically acceptable salts thereof, R₂ is a group of the formula:

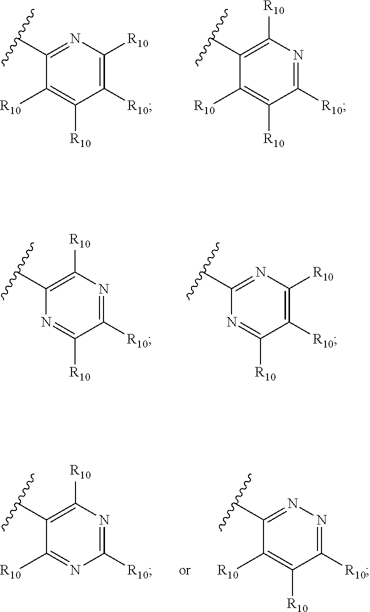

R₂ is an optionally substituted 5-member ring heteroaryl selected from pyrrole, furan, thiophene, pyrazole, isoxazole, isothiazole, imidazole, oxazole, thiazole, triazole, tetrazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —C(O)OC₁-C₃ alkyl, —CONR₁₁R₁₁, —NR₁₁R₁₁, —NR₁₁CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH (e.g., C₁-C₆ hydroxyalkyl such as —CH₂—CH₂—OH and optionally comprising a secondary —OH such as —CH₂—CH(CH₃)—OH or a tertiary —OH such as —CH₂—C(CH₃)₂—OH), oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN.

In yet a further compound of Formula (I), (II), (III), or (IV), or pharmaceutically acceptable salts thereof, R₂ is a group of the formula:

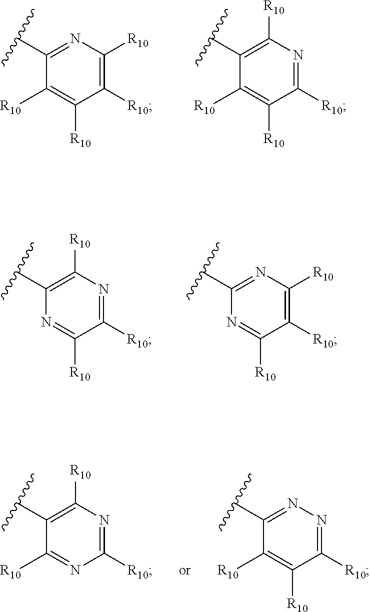

and
each R₁₀ is independently —H, —CN, halogen, C₁-C₆ haloalkyl, C₁-C₆ alkoxy, —SO₂R₁₁, an optionally substituted C₁-C₆ alkyl, an optionally substituted C₂-C₆ alkynyl, an optionally substituted C₃-C₅ cycloalkyl, or an optionally substituted heteroaryl selected from pyrazole, isoxazole, isothiazole, imidazole, oxazole, or thiazole; wherein the optionally substituted C₁-C₆ alkyl or C₂-C₆ alkynyl is optionally substituted with a —CN, —OH (e.g., C₁-C₆ hydroxyalkyl such as —CH₂—CH₂—OH and optionally comprising a secondary —OH such as —CH₂—CH(CH₃)—OH or a tertiary —OH such as —CH₂—C(CH₃)₂—OH), or C₁-C₃ alkoxy; and the optionally substituted C₃-C₅ cycloalkyl or heteroaryl is optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN.

In yet a further compound of Formula (I), (II), (III), or (IV), or pharmaceutically acceptable salts thereof, R₂ is an optionally substituted 5-member ring heteroaryl selected from pyrrole, furan, thiophene, pyrazole, isoxazole, isothiazole, imidazole, oxazole, thiazole, triazole, tetrazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —OH, —NR₁₁R₁₁, —NR₁₁CO₂R₁₁, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH (e.g., C₁-C₆ hydroxyalkyl such as —CH₂—CH₂—OH and optionally comprising a secondary —OH such as —CH₂—CH(CH₃)—OH or a tertiary —OH such as —CH₂—C(CH₃)₂—OH), halogen, oxetanyl, 2-oxabicyclo[2.1.1]hexane, C₁-C₃ alkoxy, an optionally substituted C₃-C₅ cycloalkyl, —CONR₁₁R₁₁, or —S(O)₂CH₃; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN.

In yet a further compound of Formula (I), (II), (III), or (IV), or pharmaceutically acceptable salts thereof, is an optionally substituted bicyclic ring selected from 1,3-benzodioxole, 2,3-dihydro-1,4-benzodioxine, quinoxaline, quinazoline, indole, indazole, isoindazole, benzimidazole, benzotriazole, benzothiazole, benzoxazole, benzotriazole, benzofuran, benzofurazan, pyridofurazan, quinoline, 1,5-naphthyridine, isoindolin-1-one, indolin-2-one, benzomorpholine, benzo[d]oxazol-2(3H)-one, imidazo[1,2-a]pyridine, 1,3-dihydro-2H-pyrrolo[2,3-b]pyridin-2-one, [1,3]dioxolo[4,5-b]pyridine, 6,7-dihydro-4H-pyrazolo[5,1-c][1,4]oxazine, 1,2,3,4-tetrahydroisoquinoline, 4,5,6,7-tetrahydropyrazolo[1,5-a]pyrazine, pyrazolo[4,3-b]pyridine, pyrazolo[3,4-b]pyridine, pyrazolo[3,4-c]pyridine, pyrazolo[1,5-a]pyrimidine, oxazolo[4,5-b]pyridine, oxazolo[5,4-b]pyridine, thiazolo[5,4-b]pyridine, 2,1,3-benzothiadiazole, imidazo[1,2-a]pyrimidine, or 2,3-dihydro-[1,4]dioxino[2,3-b]pyridine, 6,7-dihydro-5H-cyclopent[b]pyridine, 5,6-dihydro-4H-pyrrolo[1,2-b]pyrazole, 3,4-dihydro-2(1H)quinolinone, 2H-1,4-benzoxazin-3(4H)-one, 2-hydroxyquinoline, 3,4-dihydroisoquinolin-1,(2H)-one, 1-hydroxyisoquinoline, 1,4-dihydro-3(2H)-isoquinolinone, 3H-imidazo[4,5-b]pyridine, 4,5-dihydro-7H-pyrazolo[1,5-c]1,3]oxazine, 6,7-dihydro-5H-pyrazolo[5,1-b][1,3]oxazine, furo[3,2-c]pyridine, furo[3,2-b]pyridine, 2,3-dihydropyrazolo[5,1-b]oxazole, 5H,6H,8H-imidazo[2,1-c][1,4]oxazine, pyrazolo[1,5-b]pyridazine, imidazo[1,2-b]pyridazine, 2,4-dihydro-1H-benzo[d][1,3]oxazine, 5-oxaspiro[2,3]hexane, imidazo[1,5-a]pyridine, pyrazolo[1,5-a]pyridine; or an optionally substituted bicyclic heteroaryl of 8 to 10 ring atoms containing 1, 2, 3, 4, or 5 ring heteroatoms independently selected from N, O, or S; wherein the optionally substituted bicyclic ring is optionally substituted with one to three substituents each independently selected from halogen and C₁-C₆ alkyl; or wherein the optionally substituted bicyclic ring or the optionally substituted bicyclic heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —NR₁₁R₁₁, —NR₁₁CO₂R₁₁, —NR₁₁C(O)R₁₁, —OH, oxetanyl, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH (e.g., C₁-C₆ hydroxyalkyl such as —CH₂—CH₂—OH and optionally comprising a secondary —OH such as —CH₂—CH(CH₃)—OH or a tertiary —OH such as —CH₂—C(CH₃)₂—OH), oxetanyl, C₁-C₃ alkoxy, —CONR₁₁R₁₁, phenyl, or an optionally substituted C₃-C₅ cycloalkyl; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN.

In yet a further compound of Formula (I), (II), (III), or (IV), or pharmaceutically acceptable salts thereof, R₂ is an optionally substituted bicyclic ring selected from 1,3-benzodioxole, 2,3-dihydro-1,4-benzodioxine, isoindolin-1-one, indolin-2-one, benzo[d]oxazol-2(3H)-one, 1,3-dihydro-2H-pyrrolo[2,3-b]pyridin-2-one, or 2,3-dihydro-[1,4]dioxino[2,3-b]pyridine, or an optionally substituted bicyclic heteroaryl of 8 to 10 ring atoms containing 1, 2, 3, 4, or 5 ring heteroatoms independently selected from N, O, or S; wherein the optionally substituted bicyclic ring is optionally substituted with one to three substituents each independently selected from halogen and C₁-C₆ alkyl; the optionally substituted bicyclic heteroaryl is optionally substituted with one to three substituents each independently selected from —CN, halogen, C₁-C₆ haloalkyl, C₁-C₆ alkoxy, C₁-C₆ haloalkoxy, —SO₂R₁₀, —CONR₁₀R₁₀, —NR₁₀R₁₀, —NR₁₀CO₂R₁₀, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH (e.g., C₁-C₆ hydroxyalkyl such as —CH₂—CH₂—OH and optionally comprising a secondary —OH such as —CH₂—CH(CH₃)—OH or a tertiary —OH such as —CH₂—C(CH₃)₂—OH), oxetanyl, or C₁-C₃ alkoxy; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₀, —NR₁₀R₁₀, —OH or —CN.

In yet a further compound of Formula (I), (II), (III), or (IV), or pharmaceutically acceptable salts thereof, R₂ is a cyclopropyl, cyclobutyl, cyclopentyl, or cyclohexyl, each optionally substituted with one to three R₁₀ substituents, or R₂ is a group of the formula:

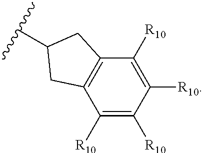

In yet a further compound of Formula (I), (II), (III), or (IV), or pharmaceutically acceptable salts thereof, R₂ is

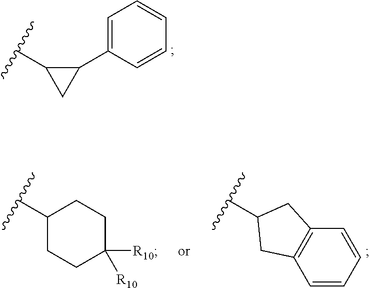

wherein each R₁₀ is independently —H, halogen, C₁-C₆ alkyl, or C₁-C₆ haloalkyl.

In yet a further compound of Formula (I), (II), (III), or (IV), or pharmaceutically acceptable salts thereof, R₂ is a group of the formula:

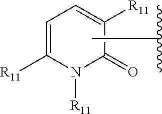

wherein R₁₁ is selected from hydrogen, alkyl, cycloalkyl, and haloalkyl.

In yet a further compound of Formula (I), (II), (III), or (IV), or pharmaceutically acceptable salts thereof, R₂ is pyrazole optionally substituted with alkyl or triazole optionally substituted with alkyl.

In yet a further compound of Formula (I), the compound is selected from:

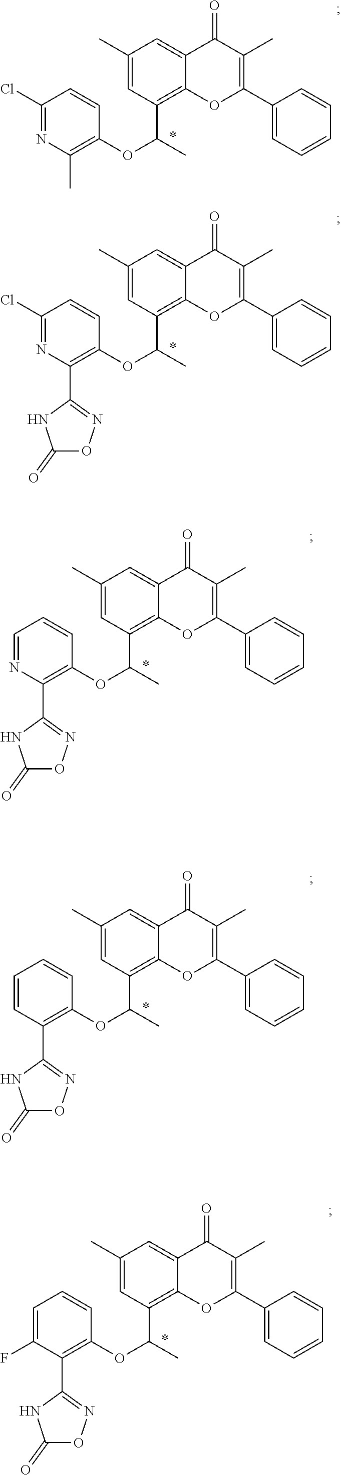 [SMILES: Cc1cc(C(C)Oc2ccc(Cl)nc2C)c2oc(-c3ccccc3)c(C)c(=O)c2c1 ; Cc1cc(C(C)Oc2ccc(Cl)nc2-c2noc(=O)[nH]2)c2oc(-c3ccccc3)c(C)c(=O)c2c1 ; Cc1cc(C(C)Oc2cccnc2-c2noc(=O)[nH]2)c2oc(-c3ccccc3)c(C)c(=O)c2c1 ; Cc1cc(C(C)Oc2ccccc2-c2noc(=O)[nH]2)c2oc(-c3ccccc3)c(C)c(=O)c2c1 ; Cc1cc(C(C)Oc2cccc(F)c2-c2noc(=O)[nH]2)c2oc(-c3ccccc3)c(C)c(=O)c2c1]

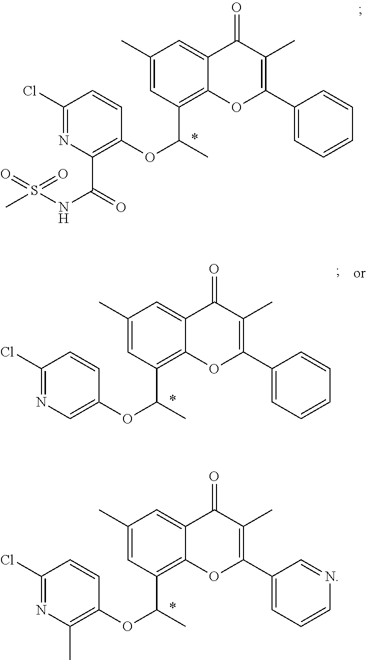 [SMILES: Cc1cc(C(C)Oc2ccc(Cl)nc2C(=O)NS(C)(=O)=O)c2oc(-c3ccccc3)c(C)c(=O)c2c1 ; Cc1cc(C(C)Oc2ccc(Cl)nc2)c2oc(-c3ccccc3)c(C)c(=O)c2c1 ; Cc1cc(C(C)Oc2ccc(Cl)nc2C)c2oc(-c3cccnc3)c(C)c(=O)c2c1]

or a pharmaceutically acceptable salt of any of the foregoing;
wherein the bond at the * position is as represented, or

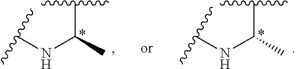

In a further embodiment of the compound of Formula (I), (II), (III), or (IV) or a pharmaceutically acceptable salt thereof, the compound is an isotopic derivative of any one of the compounds described herein or a pharmaceutically acceptable salt thereof. It is understood that the isotopic derivative can be prepared using any of a variety of art-recognized techniques. For example, the isotopic derivatives can generally be prepared by carrying out the procedures disclosed in the schemes and/or in the examples described herein or a pharmaceutically acceptable salt thereof, by substituting an isotopically labeled reagent for a non-isotopically labeled reagent. In the compounds of this invention any atom not specifically designated as a particular isotope is meant to represent a stable isotope of that atom.

In a further embodiment of a compound of Formula (I), (II), (III), or (IV) or a pharmaceutically acceptable salt thereof, the compound is deuterated at one or more positions. Unless otherwise stated, when an atom is designated specifically as “H” or “hydrogen”, the atom is understood to have hydrogen at its natural abundance isotopic composition. Also, unless otherwise stated, when an atom is designated specifically as “D” or “deuterium”, the atom is understood to have deuterium at an abundance substantially greater than the natural abundance of deuterium, which is 0.015%.

### Activities of the Disclosed Compounds

The disclosed compounds may have one or more activities as disclosed herein.

In one aspect, the disclosed compounds may inhibit the activity of PI3K-Alpha kinase and may be characterized as having (PIK3CA) inhibitory activity. In one aspect, the disclosed compounds may inhibit the activity of wild-type PIK3CA. In one aspect, the disclosed compounds may inhibit the activity of a mutant PIK3CA having one or more amino acid substitutions, deletions, or insertions relative to the amino acid sequence of wild-type PIK3CA. In one aspect, the disclosed compounds may inhibit the activity of a H1047R mutant PIK3CA. In one aspect, the disclosed compounds may inhibit the activity of an E545K mutant PIK3CA. In one aspect, the disclosed compounds may inhibit the activity of both of a H1047R mutant PIK3CA and an E545K mutant PIK3CA. In one aspect, the disclosed compounds may inhibit the activity of a wild-type PI3KCA or mutant PI3KCA as measured in an in vitro cell-based assay such as the assay entitled “PI3K-Alpha kinase (PI3Kα) activity: wild-type PI3Kα, H1047R mutant PI3Kα, and E545K mutant PI3Kα in vitro cell based assays and determination of IC50 values for inhibitors” as disclosed herein as an Example. In one aspect, the disclosed compounds may inhibit the activity of a wild-type PI3KCA and/or a mutant PI3KCA as measured in an in vitro cell-based assay and an IC50 may be determined with respect to inhibition.

In one aspect, the disclosed compounds may selectively inhibit the activity of a mutant PI3KCA relative to wild-type PI3KCA, such as a H1047R mutant PI3KCA or an E545K mutant PI3KCA, relative to wild-type PI3KCA. In one aspect, the disclosed compounds may have an IC50 for a mutant PI3KCA, such as an in vitro assay as disclosed herein, which is less than about 500 nM, 100 nM, 50 nM, 40 nM, 30 nM, 20 nM, 10 nM, or 5 nM. In one aspect, the disclosed compounds may have an IC50 for a H1047R mutant PI3KCA that is less than about 500 nM, 100 nM, 50 nM, 40 nM, 30 nM, 20 nM, 10 nM, or 5 nM. In one aspect, the disclosed compounds may have an IC50 for an E545K mutant PI3KCA that is less than about 500 nM, 450 nM, 400 nM, 350 nM, 300 nM, 250 nM, 200 nM, 150 nM, 100 nM, or 50 nM. In one aspect, the disclosed compounds may have an IC50 for wild-type PI3KCA which is greater than about 100 nM, 200 nM, 300 nM, 400 nM, 500 nM, 600 nM, 700 nM, 800 nM, 900 nM, 1000 nM, 2000 nM, or 3000 nM. In one aspect, the disclosed compounds may selectively inhibit the activity of a mutant PI3KCA relative to wild-type PI3KCA and the compounds may have an IC50 for a mutant PI3KCA in an assay, such as an in vitro assay as disclosed herein, which is at least 5×, 10×, 20×, 30×, 40×, 50×, 60×, 70×, 80×, 90×, 100×, 200×, or 500× lower than an IC50 for wild-type PI3KCA in the in vitro cell-based assay. In one aspect, the compound selectively inhibits the activity of a mutant PI3KCA relative to wild-type PI3KCA in an in vitro cell-based assay and the compound has fold selectivity for the mutant PI3KCA of at least 5×, 10×, 20×, 30×, 40×, 50×, 60×, 70×, 80×, 90×, 100×, 200×, or 500× relative to wild-type PI3KCA.

In one aspect, the disclosed compounds may exhibit metabolic stability. In one aspect, the disclosed compounds may exhibit metabolic stability as measured in one or more in vitro clearance assays as disclosed herein.

In one aspect, the disclosed compounds may exhibit metabolic stability as measured in an in vitro clearance assay that utilizes liver microsomes such as the assay entitled “Metabolic Stability and Intrinsic Clearance in Liver Microsomes” as disclosed herein as an Example. Suitable liver microsomes for use in an in vitro clearance assay may include, but are not limited to, human liver microsomes, dog liver microsomes, monkey liver microsomes, rat liver microsomes, and mouse liver microsomes. In one aspect, the disclosed compounds may have an intrinsic clearance value in an in vitro liver microsome assay (CLᵢₙₜ) which is less than about 1000, 500, 400, 300, 200, 150, 100, 90, 80, 70, 60, 50, 45, 40, 35, 30, 25, 20, 15, 10, 5, 4, 3, 2, or 1 μl/min/mg. In one aspect, scaling factors as known in the art may be utilized to calculate a Scaled-up CLₕₑₚ (mL/min/kg), a Predicted CLᵢₙₜ(mL/min/kg), and a Hepatic Extraction Ratio ((ER) (i.e., ratio of hepatic clearance to hepatic blood flow) for a given species, including but not limited to human, monkey, dog, rat and mouse.

In one aspect, the disclosed compounds may exhibit metabolic stability as measured in an in vitro clearance assay that utilizes hepatocytes such as the assay entitled “Metabolic Stability and Intrinsic Clearance in Hepatocytes” as disclosed herein as an Example. Suitable hepatocytes for use in an in vitro clearance assay may include, but are not limited to, human hepatocytes, dog hepatocytes, monkey hepatocytes, rat hepatocytes, and mouse hepatocytes. In one aspect, the disclosed compounds may have an intrinsic clearance value in an in vitro hepatocyte stability assay (CLᵢₙₜ) which is less than about 200, 150, 100, 90, 80, 70, 60, 50, 45, 40, 35, 30, 25, 20, 15, 10, 5, 4, 3, 2, or 1 μl/min/1×10⁶ cells. In one aspect, scaling factors as known in the art may be utilized to calculate a Scaled-up CLᵢₙₜ(mL/min/kg), a Predicted Hepatic Predicted Hepatic CL_{H} (mL/min/kg), and a Hepatic Extraction Ratio ((ER) (i.e., ratio of hepatic clearance to hepatic blood flow) for a given species, including but not limited to human, monkey, dog, rat and mouse.

In one aspect, the disclosed compounds do not induce expression of a cytochrome P450 enzyme in comparison to a control or vehicle in an in vitro assay, or the compounds induce expression of a cytochrome P450 enzyme at a relatively low level in comparison to a control or vehicle in an in vitro assay. In one aspect, the disclosed compounds do not induce expression of a cytochrome P450 enzyme or induce expression of a cytochrome P450 enzyme at a relatively low level compared to a control or vehicle in an in vitro assay, such as the assay entitled “In Vitro Estimation of CYP1A2, CYP2B6 and CYP3A4 Induction Potention of Primary Human Hepatocytes” as disclosed herein as an Example. Fold induction of CYP expression may be determined by measuring CYP mRNA in a test sample versus a control or vehicle sample and/or by measuring CYP enzyme activity in a test sample versus a control or vehicle sample. In one aspect, the disclosed compounds do not induce expression of CYP3A4 compared to a control or vehicle at a level of more than about 4.0×, 3.8×, 3.6×, 3.4×, 3.2×, 3.0×, 2.8×, 2.6×, 2.4×, 2.2×, 2.0×, 1.8×, 1.6×, 1.4×, 1.2×, in an in vitro assay as disclosed herein when the disclosed compounds are administered in the in vitro assay at a concentration of about 1 μl.

In one aspect, the disclosed compounds do not cause adverse events (AEs) such as hyperglycemia and diarrhea when the PI3Kα inhibitors are administered at an effective dose for inhibiting the activity of PI3Kα and treating a disease or disorder associated with PI3Kα activity. In one aspect, the disclosed compounds do not cause adverse events (AEs) such as hyperglycemia and diarrhea when the PI3Kα inhibitors are administered at an effective dose for treating cancer and resulting in tumor regression.

### Pharmaceutical Salts

A pharmaceutically acceptable salt of a compound of the present invention is, for example, an acid-addition salt of a compound of the invention, which is sufficiently basic, for example, an acid-addition salt with, for example, an inorganic or organic acid, for example hydrochloric, hydrobromic, sulfuric, phosphoric, trifluoroacetic, formic, citric, methane sulfonate or maleic acid. In addition, a pharmaceutically acceptable salt of a compound of the present invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine. Pharmaceutically acceptable salts, and common methodology for preparing them are well known in the art (see, e.g., P. Stahl, et al. Handbook of Pharmaceutical Salts: Properties, Selection, and Use, 2^{nd} Revised Edition (Wiley-VCH, 2011); S. M. Berge, et al., “Pharmaceutical Salts,” Journal of Pharmaceutical Sciences, Vol. 66, No. 1, January 1977).

Further representative “pharmaceutically acceptable salts” include, e.g., water-soluble, and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium, calcium edetate, camsylate, carbonate, chloride, citrate, clavulanate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate, pantothenate, phosphate/diphosphate, picrate, polygalacturonate, propionate, p-toluenesulfonate, salicylate, stearate, subacetate, succinate, sulfate, sulfosalicylate, tannate, tartrate, teoclate, tosylate, triethiodide, and valerate salts.

The compounds of the present invention can be prepared in a number of ways well known to those skilled in the art of organic synthesis. By way of example, compounds of the present invention can be synthesized using the methods described below, together with synthetic methods known in the art of synthetic organic chemistry, or variations thereon as appreciated by those skilled in the art. Preferred methods include but are not limited to those methods described below.

### Further Aspects

The following are further numbered aspects of the invention:
1. A compound of the formula:

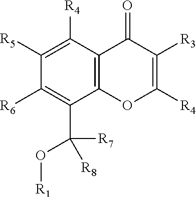

or pharmaceutically acceptable salt thereof, wherein:
R₁ is an optionally substituted bicyclic ring selected from isobenzofuranone, benzofuranone, isoindolinone, indolinone, quinazolinone, 3,4-dihydro-2H-isoquinolin-1-one, 2H-isoquinolin-1-one, imidaza[1,2-a]pyridine, or benzothiazolone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
R₁ is a group of the formula:

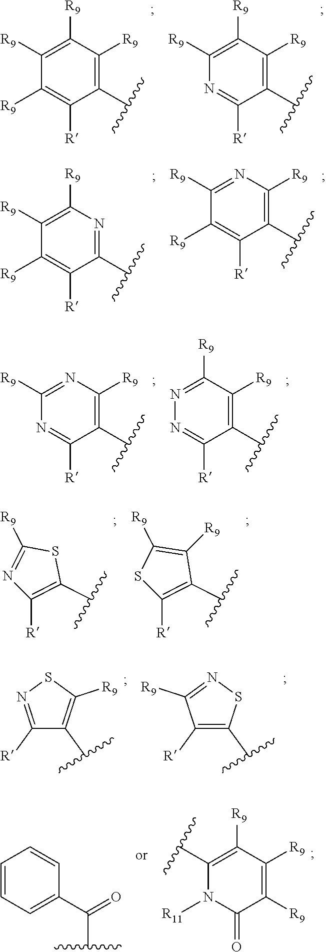

R′ is hydrogen, halogen, C₁-C₃ alkyl, C₁-C₃ haloalkyl, C₁-C₃ hydroxyalkyl, C₁-C₃ alkoxy, C₁-C₃ haloalkoxy, —OH, —CH(OH)—CH₂OH, —CH(OH)C₁-C₃ haloalkyl, —CO—CH₂OH, C₃-C₆ cycloalkyl, —NO₂, —NR₁₁R₁₁, —N(R₁₁)—CO₂C₁-C₃ alkyl, —N(R)—SO₂C₁-C₃ alkyl, —N(R)—SO₂R₁₅, —SO₂C₁-C₃ alkyl, —SOC₁-C₃ alkyl, —SO₂NR₁₁R₁₁, —SO₂NR₁₁R₁₂, —SO₂N(R)—CO—C₁-C₃ alkyl, —SO₂N(R₁₁)—CN, —C(═N—OH)—NH₂, —CN, —CONR₁₁R₁₂, —CON(R)—(CH₂)ₙ—R₁₃, —CO—SR₁₂, —CO—NHSO₂R₁₆, —COCH═SOR₁₁(R₁₁), or —COCH₂CN, or a group of the formula:

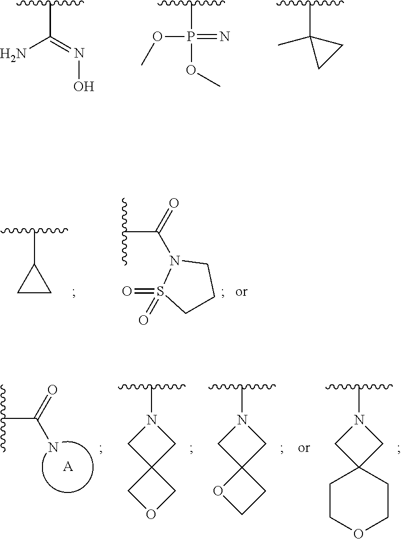

wherein ring A is pyrrolidine optionally substituted with a —CN; or
R′ is selected from oxetane, azetidine, pyrrolidine, tetrahydrofuran, morpholine, thiomorpholine, piperidine, piperazine, pyrrole, furan, thiophene, pyrazole, imidazole, isoxazole, oxazole, isothiazole, thiazole, triazole, oxadiazole, 1,2,4-oxadiazolin-5-one, 1,4-oxazepane, thiadiazole, tetrazole, phenyl, pyridine, pyridazine, pyrimidine, pyrazine, oxazepane, or triazine; each of which is optionally substituted with one to three substituents independently selected from oxo, —OH, —NR₁₁R₁₁, —N(R₁₁)—CO—R₁₁, —N(R₁₁)—CN, —OR₁₁, —CN, halogen, morpholino, oxetane, C₁-C₆ haloalkyl or C₁-C₆ alkyl optionally substituted with —OH, an aryl, a 5-member heteroaryl or a 6-member heteroaryl; or
R′ is a group of the formula:

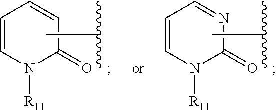

R₂ is a group of the formula:

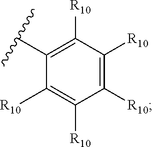

R₂ is a group of the formula:

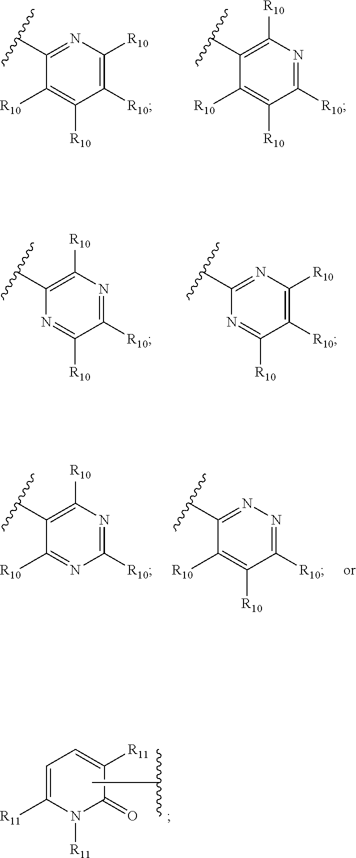

or
R₂ is an optionally substituted 5-member ring heteroaryl selected from pyrrole, furan, thiophene, pyrazole, isoxazole, isothiazole, imidazole, oxazole, thiazole, triazole, tetrazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —OH, —NR₁₁R₁₁, —NR₁₁CO₂R₁₁, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH (e.g., C₁-C₆ hydroxyalkyl such as —CH₂—CH₂—OH and optionally comprising a secondary —OH such as —CH₂—CH(CH₃)—OH or a tertiary —OH such as —CH₂—C(CH₃)₂—OH), halogen, oxetanyl, 2-oxabicyclo[2.1.1]hexane, C₁-C₃ alkoxy, an optionally substituted C₃-C₅ cycloalkyl, —CONR₁₁R₁₁, or —S(O)₂CH₃; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is an optionally substituted bicyclic ring selected from 1,3-benzodioxole, 2,3-dihydro-1,4-benzodioxine, quinoxaline, quinazoline, indole, indazole, isoindazole, benzimidazole, benzotriazole, benzothiazole, benzoxazole, benzotriazole, benzofuran, benzofurazan, pyridofurazan, quinoline, 1,5-naphthyridine, isoindolin-1-one, indolin-2-one, benzomorpholine, benzo[d]oxazol-2(3H)-one, imidazo[1,2-a]pyridine, 1,3-dihydro-2H-pyrrolo[2,3-b]pyridin-2-one, [1,3]dioxolo[4,5-b]pyridine, 6,7-dihydro-4H-pyrazolo[5,1-c][1,4]oxazine, 1,2,3,4-tetrahydroisoquinoline, 4,5,6,7-tetrahydropyrazolo[1,5-a]pyrazine, pyrazolo[4,3-b]pyridine, pyrazolo[3,4-b]pyridine, pyrazolo[3,4-c]pyridine, pyrazolo[1,5-a]pyrimidine, oxazolo[4,5-b]pyridine, oxazolo[5,4-b]pyridine, thiazolo[5,4-b]pyridine, 2,1,3-benzothiadiazole, imidazo[1,2-a]pyrimidine, or 2,3-dihydro-[1,4]dioxino[2,3-b]pyridine, 6,7-dihydro-5H-cyclopent[b]pyridine, 5,6-dihydro-4H-pyrrolo[1,2-b]pyrazole, 3,4-dihydro-2(1H)quinolinone, 2H-1,4-benzoxazin-3(4H)-one, 2-hydroxyquinoline, 3,4-dihydroisoquinolin-1,(2H)-one, 1-hydroxyisoquinoline, 1,4-dihydro-3(2H)-isoquinolinone, 3H-imidazo[4,5-b]pyridine, 4,5-dihydro-7H-pyrazolo[1,5-c]1,3]oxazine, 6,7-dihydro-5H-pyrazolo[5,1-b][1,3]oxazine, furo[3,2-c]pyridine, furo[3,2-b]pyridine, 2,3-dihydropyrazolo[5,1-b]oxazole, 5H,6H,8H-imidazo[2,1-c][1,4]oxazine, pyrazolo[1,5-b]pyridazine, imidazo[1,2-b]pyridazine, 2,4-dihydro-1H-benzo[d][1,3]oxazine, 5-oxaspiro[2,3]hexane, imidazo[1,5-a]pyridine, pyrazolo[1,5-a]pyridine; or an optionally substituted bicyclic heteroaryl of 8 to 10 ring atoms containing 1, 2, 3, 4, or 5 ring heteroatoms independently selected from N, O, or S; wherein the optionally substituted bicyclic ring is optionally substituted with one to three substituents each independently selected from halogen and C₁-C₆ alkyl; or wherein the optionally substituted bicyclic ring or the optionally substituted bicyclic heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —NR₁₁R₁₁, —NR₁₁CO₂R₁₁, —NR₁₁C(O)R₁₁, —OH, oxetanyl, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH (e.g., C₁-C₆ hydroxyalkyl such as —CH₂—CH₂—OH and optionally comprising a secondary —OH such as —CH₂—CH(CH₃)—OH or a tertiary —OH such as —CH₂—C(CH₃)₂—OH), oxetanyl, C₁-C₃ alkoxy, —CONR₁₁R₁₁, phenyl, or an optionally substituted C₃-C₅ cycloalkyl; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is a cyclopropyl, cyclobutyl, cyclopentyl, bicyclo[1.1.1]pentane, bicyclo[2.2.2]octane, or cyclohexyl, each optionally substituted with one to three R₁₀ substituents, or R₂ is a group of the formula:

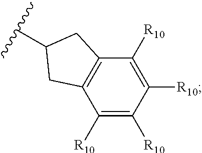

R₂ is a heterocycloalkyl, optionally oxetane substituted with one to three R₁₀ substituents;
R₃ is —H, halogen, —CN, —C(CN)═CHOH, —N(H)(C₁-C₃ alkyl), —N(C₁-C₃ alkyl)₂, —N(H)(CH₂CH₂CO₂H), —CO—C₁-C₃ alkyl, C₁-C₆ alkyl, C₁-C₆ haloalkyl, C₁-C₆ hydroxyalkyl, C₃-C₅ cycloalkyl, an optionally substituted heterocycle of 3 to 5 ring atoms containing 1, 2, or 3 ring heteroatoms independently selected from N, O, or S, or an optionally substituted heteroaryl of 5 or 6 ring atoms containing 1, 2, or 3 ring heteroatoms independently selected from N, O, or S; wherein the optionally substituted heterocycle or heteroaryl is each optionally substituted with one to three substituents each independently selected from halogen, C₁-C₃ alkyl, or C₁-C₃ haloalkyl;
each of R₄, R₅ and R₆ is independently —H, halogen, —CN, C₁-C₆ alkyl or C₁-C₆ haloalkyl;
R₇ is —CN, C₁-C₆ alkyl, CH₂OH, or C₁-C₆ haloalkyl;
R₈ is —H or C₁-C₆ alkyl;
each R₉ is independently —H, halogen, —CN, C₁-C₆ alkyl, C₁-C₆ haloalkyl, C₁-C₆ alkoxy, or C₃-C₅ cycloalkyl;
each R₁₀ is independently —H, —CN, halogen, C₁-C₆ haloalkyl, C₁-C₆ alkoxy, C₁-C₆ haloalkoxy, —SO₂R₁₁, —SONR₁₁R₁₁, —CO₂H, —CO₂C₁-C₃ alkyl, —CONR₁₁R₁₂, —NR₁₁R₁₁, —NR₁₁—CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, an optionally substituted heteroaryl selected from pyrazole, isoxazole, isothiazole, imidazole, oxazole, thiazole, or pyridine, or a group of the formula:

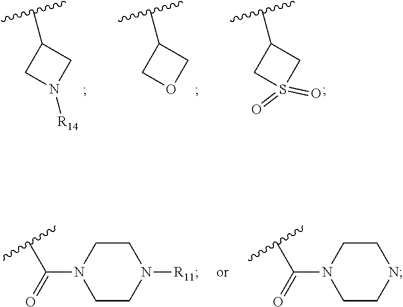

wherein the optionally substituted C₁-C₆ alkyl, C₂-C₆ alkenyl, or C₂-C₆ alkynyl is each optionally substituted with a —CN, —OH (e.g., where R₁₀ is C₁-C₆ hydroxyalkyl such as —CH₂—CH₂—OH and optionally where R₁₀ is a hydroxyl comprising a secondary —OH such as —CH₂—CH(CH₃)—OH or a tertiary —OH such as —CH₂—C(CH₃)₂—OH), oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN;
each R₁₁ is independently —H, C₁-C₃ alkyl, C₃-C₇ cycloalkyl, or C₁-C₃ haloalkyl;
each R₁₂ is independently —H, optionally substituted C₁-C₃ alkyl, C₃-C₆ cycloalkyl, —SO₂C₁-C₃ alkyl, —SO₂C₁-C₃ haloalkyl, —SO₂NR₁₁R₁₁, —NR₁₁R₁₁, —OR₁₁, —O—CH₂—CH(OH)—CH₂OH, —CN, oxetane, tetrahydrofuran, an aryl, a 5-member heteroaryl optionally substituted with a methyl, a 6-member heteroaryl, or a group of the formula

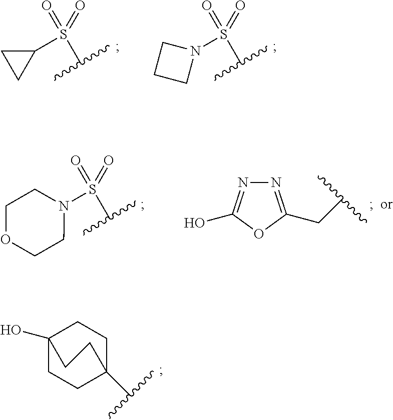 [SMILES: *S(=O)(=O)C1CC1 ; *S(=O)(=O)N1CCC1 ; *S(=O)(=O)N1CCOCC1 ; *Cc1nnc(O)o1 ; *C12CCC(O)(CC1)CC2]

wherein the optionally substituted C₁-C₃ alkyl is optionally substituted with —OH, a C₃-C₆ cycloalkyl, oxetane, tetrahydrofuran, an aryl, a 5-member heteroaryl, a 6-member heteroaryl or an indole;
R₁₃ is —NR₁₁R₁₁, —OR₁₁, —SO₂C₁-C₃ alkyl, or a ring selected from oxetane, tetrahydrofuran or oxadiazole wherein the ring is optionally substituted with —NR₁₁R₁₁, or —OR₁₁;
R₁₄ is —H, optionally substituted C₁-C₃ alkyl, —SO₂C₁-C₃ alkyl, an aryl, a 5-member heteroaryl, or a 6-member heteroaryl; wherein the optionally substituted C₁-C₃ alkyl is optionally substituted with an aryl, a 5-member heteroaryl or a 6-member heteroaryl;
R₁₅ is an optionally substituted aryl, or an optionally substituted 6-member heteroaryl; wherein the optionally substituted aryl, or the optionally substituted 6-member heteroaryl is each optionally substituted with one to three substituents each independently selected from halogen, C₁-C₃ alkyl, or C₁-C₃ haloalkyl;
R₁₆ is H, C₁-C₃ alkyl, —NH₂, phenyl, or pyridine, and
n is 0, 1 or 2.
2. A compound of the Formula:

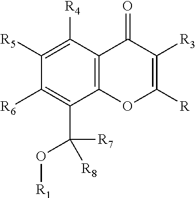

or pharmaceutically acceptable salt thereof, wherein:
R₁ is an optionally substituted bicyclic ring selected from isobenzofuranone, benzofuranone, isoindolinone, indolinone, quinazolinone, 3,4-dihydro-2H-isoquinolin-1-one, 2H-isoquinolin-1-one, or benzothiazolone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
R₁ is a group of the formula:

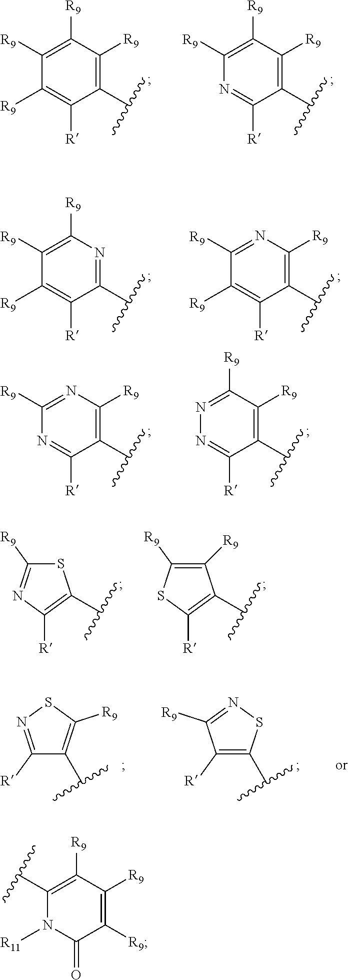

R′ is hydrogen, halogen, C₁-C₃ alkyl, C₁-C₃ haloalkyl, C₁-C₃ alkoxy, C₁-C₃ haloalkoxy, —OH, —CH(OH)—CH₂OH, —CO—CH₂OH, C₃-C₆ cycloalkyl, —NO₂, —N(R₁₁)—CO₂C₁-C₃ alkyl, —N(R₁₁)—SO₂C₁-C₃ alkyl, —N(R₁₁)—SO₂R₁₅, —SO₂C₁-C₃ alkyl, —SO₂NR₁₁R₁₁, —SO₂N(R₁₁)—CO—C₁-C₃ alkyl, —SO₂N(R₁₁)—CN, —C(═N—OH)—NH₂, —CN, —CONR₁₁R₁₂, —CON(R₁₁)—(CH₂)ₙ—R₁₃, —CO—SR₁₂, or a group of the formula:

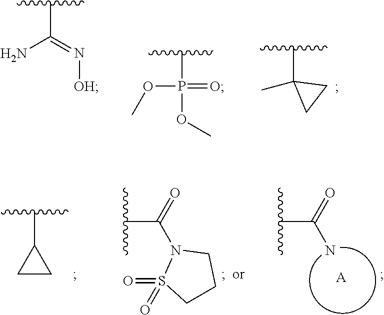

wherein ring A is pyrrolidine optionally substituted with a —CN; or
R′ is selected from oxetane, pyrrolidine, tetrahydrofuran, morpholine, piperidine, piperazine, pyrrole, furan, thiophene, pyrazole, imidazole, isoxazole, oxazole, isothiazole, thiazole, triazole, oxadiazole, thiadiazole, tetrazole, phenyl, pyridine, pyridazine, pyrimidine, pyrazine, or triazine; each of which is optionally substituted with one to three substituents independently selected from oxo, —OH, —NR₁₁R₁₁, —N(R₁₁)—CO—R₁₁, —N(R₁₁)—CN, —OR₁₁, —CN, halogen, C₁-C₆ haloalkyl or C₁-C₆ alkyl optionally substituted with an aryl, a 5-member heteroaryl or a 6-member heteroaryl; or
R′ is a group of the formula:

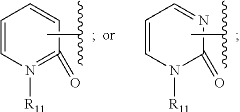

R₂ is a group of the formula:

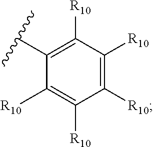

or
R₂ is a group of the formula:

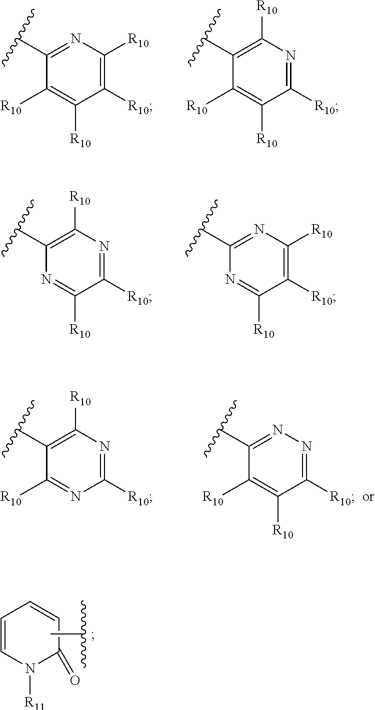

or
R₂ is an optionally substituted 5-member ring heteroaryl selected from pyrrole, furan, thiophene, pyrazole, isoxazole, isothiazole, imidazole, oxazole, thiazole, triazole, tetrazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —OH, —NR₁₁R₁₁, —NR₁₁CO₂R₁₁, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH (e.g., where R₁₀ is C₁-C₆ hydroxyalkyl such as —CH₂—CH₂—OH and optionally where R₁₀ is a hydroxyl comprising a secondary —OH such as —CH₂—CH(CH₃)—OH or a tertiary —OH such as —CH₂—C(CH₃)₂—OH), oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is an optionally substituted bicyclic ring selected from 1,3-benzodioxole, 2,3-dihydro-1,4-benzodioxine, isoindolin-1-one, indolin-2-one, benzo[d]oxazol-2(3H)-one, 1,3-dihydro-2H-pyrrolo[2,3-b]pyridin-2-one, or 2,3-dihydro-[1,4]dioxino[2,3-b]pyridine, or an optionally substituted bicyclic heteroaryl of 8 to 10 ring atoms containing 1, 2, 3, 4, or 5 ring heteroatoms independently selected from N, O, or S; wherein the optionally substituted bicyclic ring is optionally substituted with one to three substituents each independently selected from halogen and C₁-C₆ alkyl; or the optionally substituted bicyclic ring or optionally substituted bicyclic heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —NR₁₁R₁₁, —NR₁₁CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH (e.g., C₁-C₆ hydroxyalkyl such as —CH₂—CH₂—OH and optionally comprising a secondary —OH such as —CH₂—CH(CH₃)—OH or a tertiary —OH such as —CH₂—C(CH₃)₂—OH), oxetanyl, C₁-C₃ alkoxy, —CONR₁₁R₁₁ or phenyl; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is a cyclopropyl, cyclobutyl, cyclopentyl, or cyclohexyl, each optionally substituted with one to three R₁₀ substituents, or R₂ is a group of the formula:

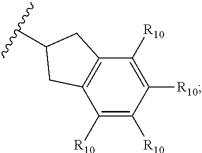

R₃ is —H, halogen, —CN, —N(H)(C₁-C₃ alkyl), —N(C₁-C₃ alkyl)₂, —N(H)(CH₂CH₂CO₂H), —CO—C₁-C₃ alkyl, C₁-C₆ alkyl, C₁-C₆ haloalkyl, C₁-C₆ hydroxyalkyl, C₃-C₅ cycloalkyl, an optionally substituted heterocycle of 3 to 5 ring atoms containing 1, 2, or 3 ring heteroatoms independently selected from N, O, or S, or an optionally substituted heteroaryl of 5 or 6 ring atoms containing 1, 2, or 3 ring heteroatoms independently selected from N, O, or S; wherein the optionally substituted heterocycle or heteroaryl is each optionally substituted with one to three substituents each independently selected from halogen, C₁-C₃ alkyl, or C₁-C₃ haloalkyl;
each of R₄, R₅ and R₆ is independently —H, halogen, C₁-C₆ alkyl or C₁-C₆ haloalkyl;
R₇ is —CN, C₁-C₆ alkyl or C₁-C₆ haloalkyl;
R₈ is —H or C₁-C₆ alkyl;
each R₉ is independently —H, halogen, —CN, C₁-C₆ alkyl, C₁-C₆ haloalkyl, C₁-C₆ alkoxy, or C₃-C₅ cycloalkyl;
each R₁₀ is independently —H, —CN, halogen, C₁-C₆ haloalkyl, C₁-C₆ alkoxy, C₁-C₆ haloalkoxy, —SO₂R₁₁, —SONR₁₁R₁₁, —CO₂H, —CO₂C₁-C₃ alkyl, —CONR₁₁R₁₂, —NR₁₁R₁₁, —NR₁₁—CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, an optionally substituted heteroaryl selected from pyrazole, isoxazole, isothiazole, imidazole, oxazole, thiazole, or pyridine, or a group of the formula:

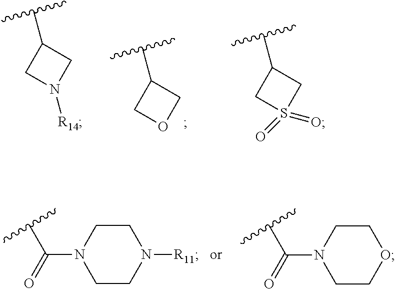

wherein the optionally substituted C₁-C₆ alkyl, C₂-C₆ alkenyl, or C₂-C₆ alkynyl is each optionally substituted with a —CN, —OH (e.g., where R₁₀ is C₁-C₆ alkyl and optionally where R₁₀ comprises a secondary —OH or a tertiary —OH), oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN;
each R₁₁ is independently —H or C₁-C₃ alkyl;
R₁₂ is —H, optionally substituted C₁-C₃ alkyl, C₃-C₆ cycloalkyl, —SO₂C₁-C₃ alkyl, —SO₂C₁-C₃ haloalkyl, —SO₂NR₁₁R₁₁, —NR₁₁R₁₁, —OR₁₁, —O—CH₂—CH(OH)—CH₂OH, —CN, oxetane, tetrahydrofuran, an aryl, a 5-member heteroaryl optionally substituted with a methyl, a 6-member heteroaryl, or a group of the formula

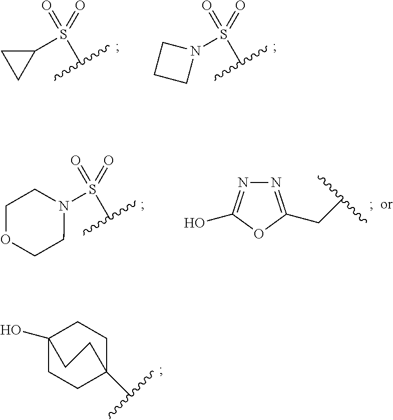 [SMILES: *S(=O)(=O)C1CC1 ; *S(=O)(=O)N1CCC1 ; *S(=O)(=O)N1CCOCC1 ; *Cc1nnc(O)o1 ; *C12CCC(O)(CC1)CC2]

wherein the optionally substituted C₁-C₃ alkyl is optionally substituted with —OH, a C₃-C₆ cycloalkyl, oxetane, tetrahydrofuran, an aryl, a 5-member heteroaryl, a 6-member heteroaryl or an indole; and
R₁₄ is —H, optionally substituted C₁-C₃ alkyl, —SO₂C₁-C₃ alkyl, an aryl, a 5-member heteroaryl, or a 6-member heteroaryl; wherein the optionally substituted C₁-C₃ alkyl is optionally substituted with an aryl, a 5-member heteroaryl or a 6-member heteroaryl.
3. The compound of aspect 1 or 2, or pharmaceutically acceptable salt thereof, wherein R₈ is —H.
4. The compound of any one of aspects 1-3, or pharmaceutically acceptable salt thereof, having the Formula:

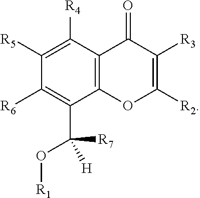

5. The compound of any one of aspects 1-4, or pharmaceutically acceptable salt thereof, wherein R₄ is —H or halogen.
6. The compound of any one of aspects 1-5, or pharmaceutically acceptable salt thereof, wherein R₄ is —H.
7. The compound of any one of aspects 1-6, or pharmaceutically acceptable salt thereof having the Formula

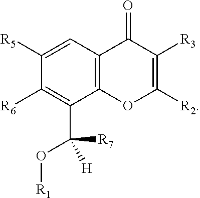

8. The compound of any one of aspects 1-7, or pharmaceutically acceptable salt thereof, wherein R₃ is —H, —CN, C₁-C₃ alkyl, or C₁-C₃ haloalkyl.
9. The compound of any one of aspects 1-8, or pharmaceutically acceptable salt thereof, wherein R₃ is —H, —CN, methyl, or trifluoromethyl.
10. The compound of any one of aspects 1-9, or pharmaceutically acceptable salt thereof, wherein R₃ is —H or methyl.
11. The compound of any one of aspects 1-10, or pharmaceutically acceptable salt thereof, wherein R₅ is —H, halogen, C₁-C₃ alkyl or C₁-C₃ haloalkyl.
12. The compound of any one of aspects 1-11, or pharmaceutically acceptable salt thereof, wherein R₅ is —H, halogen, methyl, or trifluoromethyl.
13. The compound of any one of aspects 1-12, or pharmaceutically acceptable salt thereof, wherein R₆ is —H or halogen.
14. The compound of any one of aspects 1-13, or pharmaceutically acceptable salt thereof, wherein R₇ is —CN, C₁-C₃ alkyl or C₁-C₃ haloalkyl.
15. The compound of any one of aspects 1-14, or pharmaceutically acceptable salt thereof, wherein R₇ is —CN, methyl or trifluoromethyl.
16. The compound of any one of aspects 1-15, or pharmaceutically acceptable salt thereof, wherein R₇ is methyl.
17. The compound of any one of aspects 1-16, or pharmaceutically acceptable salt thereof, having the Formula:

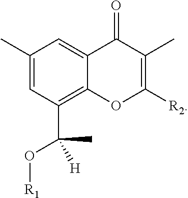

18. The compound of any one of aspects 1-17, or pharmaceutically acceptable salt thereof, wherein R₁ is a group of the formula:

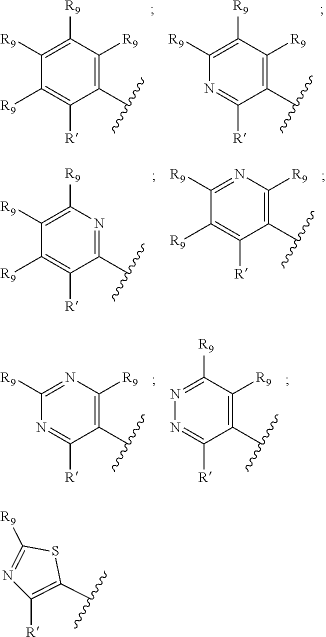

19. The compound of any one of aspects 1-18, or pharmaceutically acceptable salt thereof, wherein R₁ is a group of the formula:

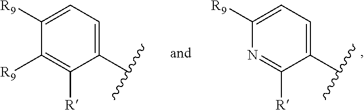

wherein R₉ is selected from —H and halogen.
20. The compound of any one of aspects 1-18, or pharmaceutically acceptable salt thereof, wherein each R₉ is independently —H, halogen, C₁-C₆ alkyl, C₁-C₆ haloalkyl, C₁-C₆ alkoxy, or C₃-C₅ cycloalkyl.
21. The compound of any one of aspects 1-18, or pharmaceutically acceptable salt thereof, wherein each R₉ is independently —H, halogen, C₁-C₃ alkyl or C₁-C₃ haloalkyl.
22. The compound of any one of aspects 1-21, or pharmaceutically acceptable salt thereof, wherein R₂ is a group of the formula:

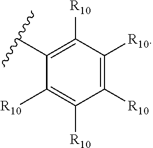

23. The compound of any one of aspects 1-22, or pharmaceutically acceptable salt thereof, wherein R₂ is a group of the formula:

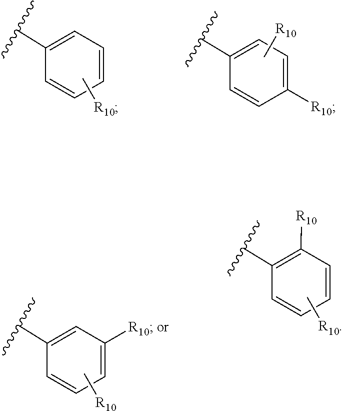

24. The compound of any one of aspects 1-21, or pharmaceutically acceptable salt thereof, wherein R₂ is a group of the formula:

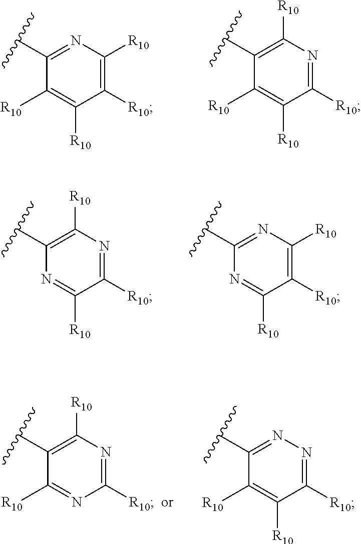

or
   R₂ is an optionally substituted 5-member ring heteroaryl selected from pyrrole, furan, thiophene, pyrazole, isoxazole, isothiazole, imidazole, oxazole, thiazole, triazole, tetrazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —C(O)OC₁-C₃ alkyl, —CONR₁₁R₁₁, —NR₁₁R₁₁, —NR₁₁CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH (e.g., C₁-C₆ hydroxyalkyl such as —CH₂—CH₂—OH and optionally comprising a secondary —OH such as —CH₂—CH(CH₃)—OH or a tertiary —OH such as —CH₂—C(CH₃)₂—OH), oxetanyl, C₁-C₃ alkoxy, optionally substituted C₃-C₅ cycloalkyl, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN.
25. The compound of any one of aspects 1-21 wherein R₂ is 1H-indazol-5-yl, 1H-indazol-6-yl, or 1H-pyrazol-4-yl, which is N-substituted with C₁-C₃ alkyl or C₁-C₆ hydroxyalkyl.
26. The compound of any one of aspects 1-21, or pharmaceutically acceptable salt thereof, wherein R₂ is a group of the formula:

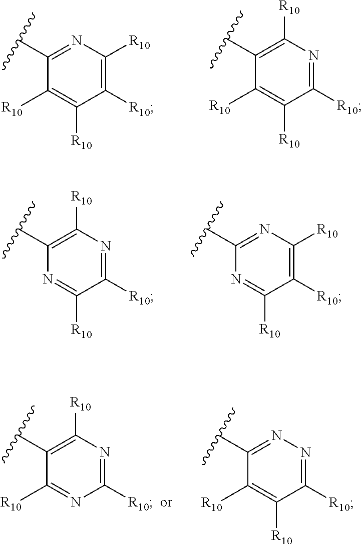

and
   each R₁₀ is independently —H, —CN, halogen, C₁-C₆ haloalkyl, C₁-C₆ alkoxy, —SO₂R₁₁, an optionally substituted C₁-C₆ alkyl, an optionally substituted C₂-C₆ alkynyl, an optionally substituted C₃-C₅ cycloalkyl, or an optionally substituted heteroaryl selected from pyrazole, isoxazole, isothiazole, imidazole, oxazole, or thiazole; wherein the optionally substituted C₁-C₆ alkyl or C₂-C₆ alkynyl is optionally substituted with a —CN, —OH (e.g., where R₁₀ is C₁-C₆ hydroxyalkyl such as —CH₂—CH₂—OH and optionally where R₁₀ is a hydroxyl comprising a secondary —OH such as —CH₂—CH(CH₃)—OH or a tertiary —OH such as —CH₂—C(CH₃)₂—OH), or C₁-C₃ alkoxy; and the optionally substituted C₃-C₅ cycloalkyl or heteroaryl is optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN.
27. The compound of any one of aspects 1-21, or pharmaceutically acceptable salt thereof, wherein R₂ is an optionally substituted bicyclic ring selected from 1,3-benzodioxole, 2,3-dihydro-1,4-benzodioxine, isoindolin-1-one, indolin-2-one, benzo[d]oxazol-2(3H)-one, 1,3-dihydro-2H-pyrrolo[2,3-b]pyridin-2-one, or 2,3-dihydro-[1,4]dioxino[2,3-b]pyridine, or an optionally substituted bicyclic heteroaryl of 8 to 10 ring atoms containing 1, 2, 3, 4, or 5 ring heteroatoms independently selected from N, O, or S; wherein the optionally substituted bicyclic ring is optionally substituted with one to three substituents each independently selected from halogen and C₁-C₆ alkyl; or the optionally substituted bicyclic ring or the optionally substituted bicyclic heteroaryl is optionally substituted with one to three substituents each independently selected from —CN, halogen, C₁-C₆ haloalkyl, C₁-C₆ alkoxy, C₁-C₆ haloalkoxy, —SO₂R₁₀, —C(O)OC₁-C₃ alkyl, —CONR₁₀R₁₀, —NR₁₀R₁₀, —NR₁₀CO₂R₁₀, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH (e.g., C₁-C₆ hydroxyalkyl such as —CH₂—CH₂—OH and optionally comprising a secondary —OH such as —CH₂—CH(CH₃)—OH or a tertiary —OH such as —CH₂—C(CH₃)₂—OH), oxetanyl, C₁-C₃ alkoxy, optionally substituted C₃-C₅ cycloalkyl, —CONR₁₀R₁₀ or phenyl; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₀, —NR₁₀R₁₀, —OH or —CN.
28. The compound of any one of aspects 1-21, or pharmaceutically acceptable salt thereof, wherein R₂ is an optionally substituted bicyclic ring selected from 1,3-benzodioxole, 2,3-dihydro-1,4-benzodioxine, isoindolin-1-one, indolin-2-one, benzo[d]oxazol-2(3H)-one, 1,3-dihydro-2H-pyrrolo[2,3-b]pyridin-2-one, or 2,3-dihydro-[1,4]dioxino[2,3-b]pyridine, or an optionally substituted bicyclic heteroaryl of 8 to 10 ring atoms containing 1, 2, 3, 4, or 5 ring heteroatoms independently selected from N, O, or S; wherein the optionally substituted bicyclic ring is optionally substituted with one to three substituents each independently selected from halogen and C₁-C₆ alkyl; or the optionally substituted bicyclic ring or the optionally substituted bicyclic heteroaryl is optionally substituted with one to three substituents each independently selected from —CN, halogen, C₁-C₆ haloalkyl, C₁-C₆ alkoxy, C₁-C₆ haloalkoxy, —SO₂R₁₀, —CONR₁₀R₁₀, —NR₁₀R₁₀, —NR₁₀CO₂R₁₀, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH (e.g., where R₁₀ is C₁-C₆ hydroxyalkyl such as —CH₂—CH₂—OH and optionally where R₁₀ is a hydroxyl comprising a secondary —OH such as —CH₂—CH(CH₃)—OH or a tertiary —OH such as —CH₂—C(CH₃)₂—OH), oxetanyl, C₁-C₃ alkoxy, or optionally substituted C₃-C₅ cycloalkyl; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₀, —NR₁₀R₁₀, —OH or —CN.
29. The compound of any one of aspects 1-21, or pharmaceutically acceptable salt thereof, wherein R₂ is a cyclopropyl, cyclobutyl, cyclopentyl, or cyclohexyl, each optionally substituted with one to three R₁₀ substituents, or R₂ is a group of the formula:

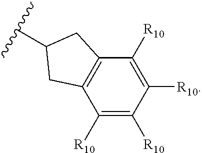

30. The compound of aspect 1 or aspect 2 selected from:

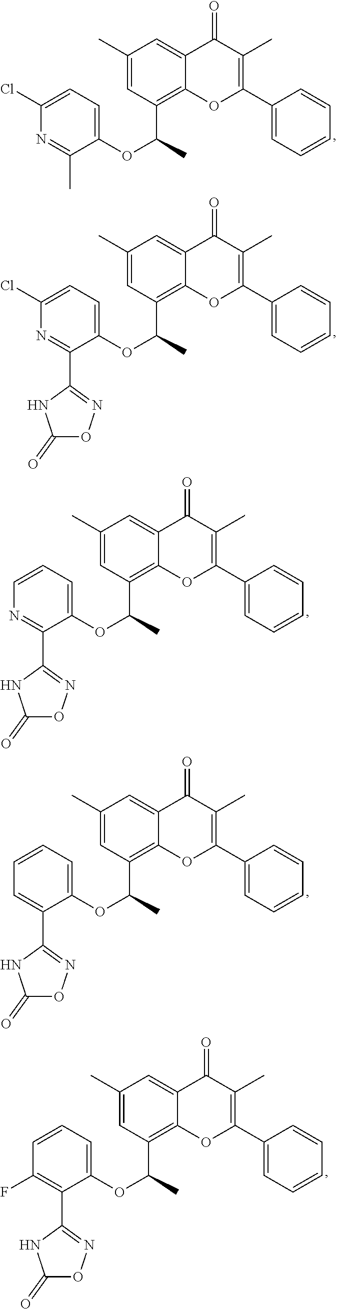 [SMILES: Cc1cc([C@@H](C)Oc2ccc(Cl)nc2C)c2oc(-c3ccccc3)c(C)c(=O)c2c1 ; Cc1cc([C@@H](C)Oc2ccc(Cl)nc2-c2noc(=O)[nH]2)c2oc(-c3ccccc3)c(C)c(=O)c2c1 ; Cc1cc([C@@H](C)Oc2cccnc2-c2noc(=O)[nH]2)c2oc(-c3ccccc3)c(C)c(=O)c2c1 ; Cc1cc([C@@H](C)Oc2ccccc2-c2noc(=O)[nH]2)c2oc(-c3ccccc3)c(C)c(=O)c2c1 ; Cc1cc([C@@H](C)Oc2cccc(F)c2-c2noc(=O)[nH]2)c2oc(-c3ccccc3)c(C)c(=O)c2c1]

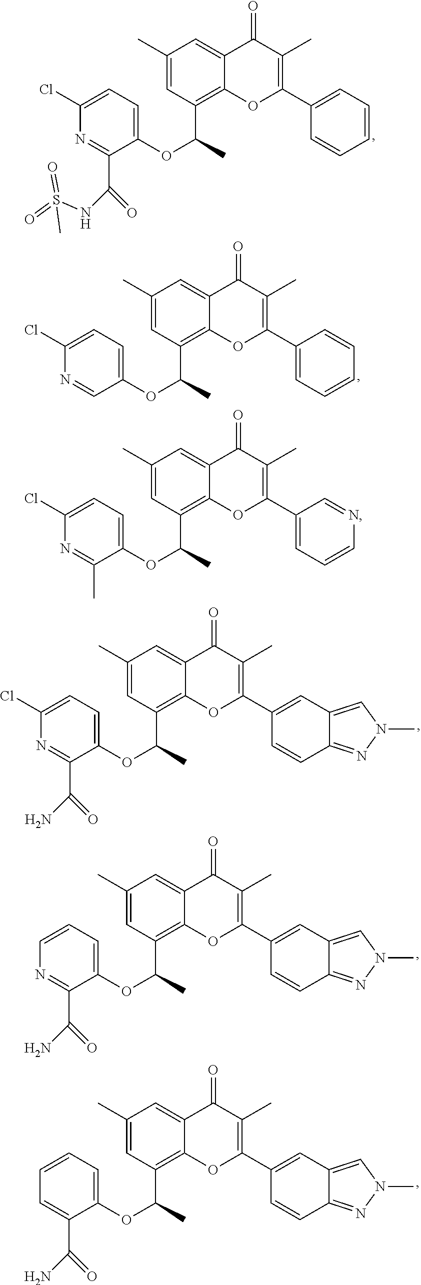

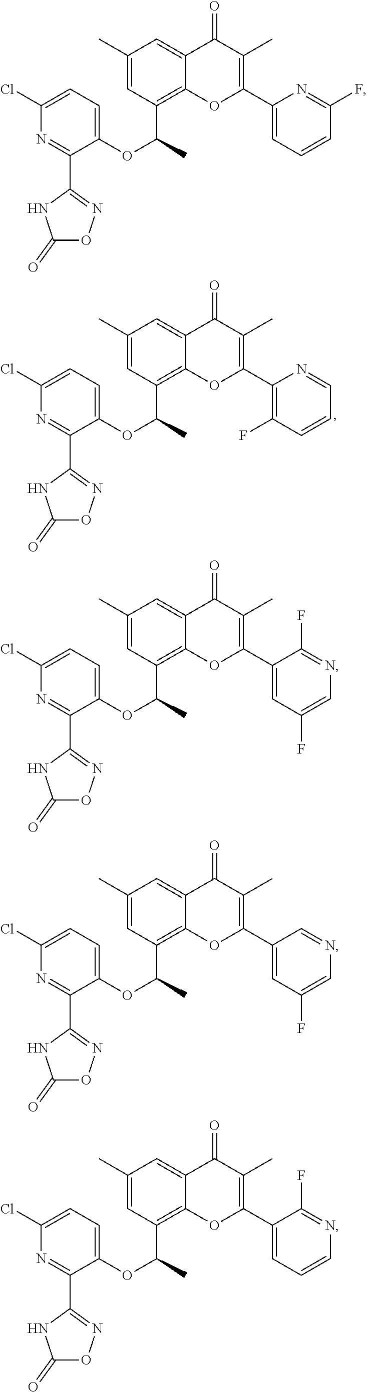 [SMILES: Cc1cc([C@@H](C)Oc2ccc(Cl)nc2-c2noc(=O)[nH]2)c2oc(-c3cccc(F)n3)c(C)c(=O)c2c1 ; Cc1cc([C@@H](C)Oc2ccc(Cl)nc2-c2noc(=O)[nH]2)c2oc(-c3ncccc3F)c(C)c(=O)c2c1 ; Cc1cc([C@@H](C)Oc2ccc(Cl)nc2-c2noc(=O)[nH]2)c2oc(-c3cc(F)cnc3F)c(C)c(=O)c2c1 ; Cc1cc([C@@H](C)Oc2ccc(Cl)nc2-c2noc(=O)[nH]2)c2oc(-c3cncc(F)c3)c(C)c(=O)c2c1 ; Cc1cc([C@@H](C)Oc2ccc(Cl)nc2-c2noc(=O)[nH]2)c2oc(-c3cccnc3F)c(C)c(=O)c2c1]

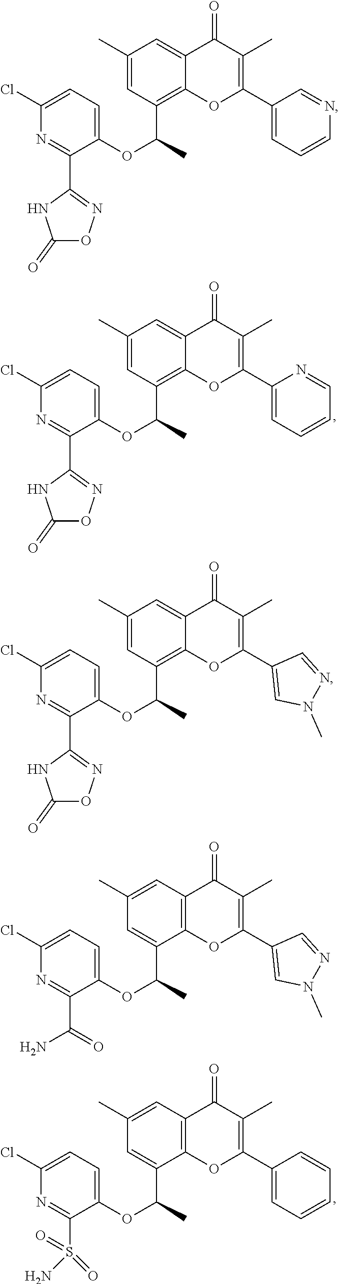 [SMILES: Cc1cc([C@@H](C)Oc2ccc(Cl)nc2-c2noc(=O)[nH]2)c2oc(-c3cccnc3)c(C)c(=O)c2c1 ; Cc1cc([C@@H](C)Oc2ccc(Cl)nc2-c2noc(=O)[nH]2)c2oc(-c3ccccn3)c(C)c(=O)c2c1 ; Cc1cc([C@@H](C)Oc2ccc(Cl)nc2-c2noc(=O)[nH]2)c2oc(-c3cnn(C)c3)c(C)c(=O)c2c1 ; Cc1cc([C@@H](C)Oc2ccc(Cl)nc2C(N)=O)c2oc(-c3cnn(C)c3)c(C)c(=O)c2c1 ; Cc1cc([C@@H](C)Oc2ccc(Cl)nc2S(N)(=O)=O)c2oc(-c3ccccc3)c(C)c(=O)c2c1]

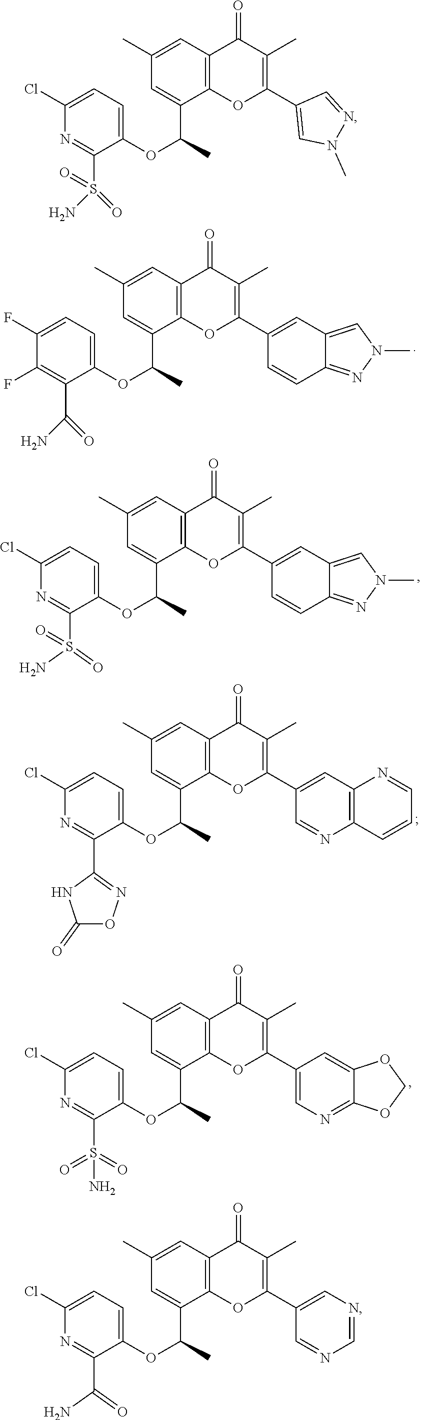

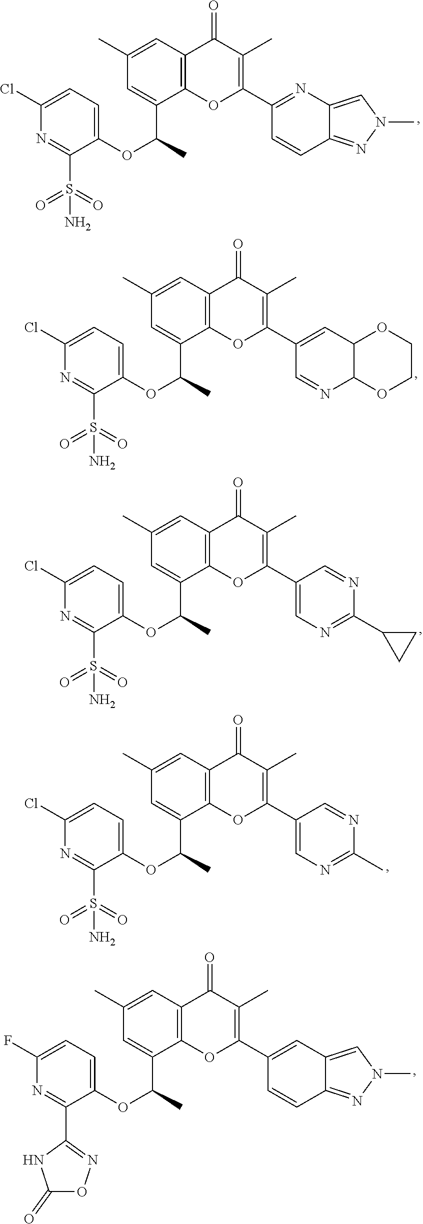

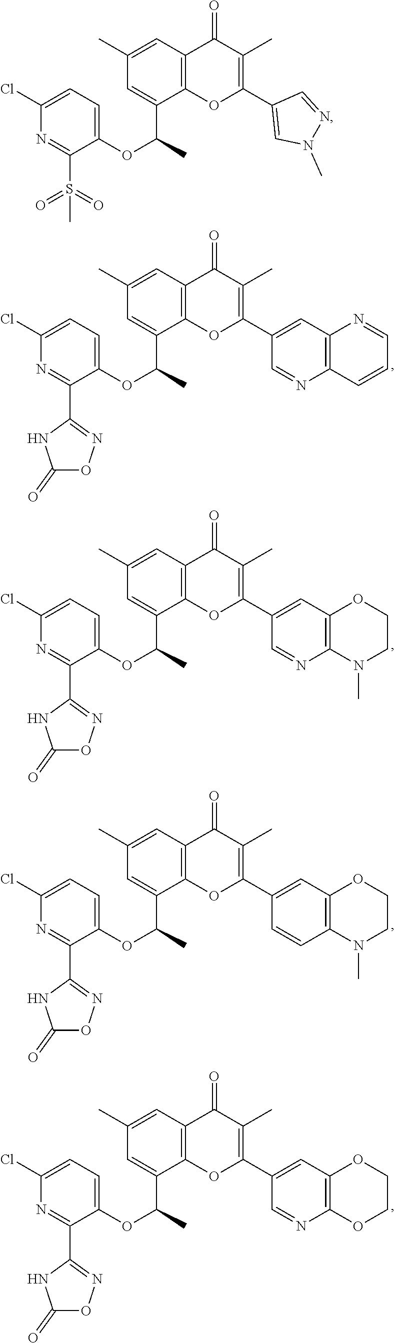 [SMILES: Cc1cc([C@@H](C)Oc2ccc(Cl)nc2S(C)(=O)=O)c2oc(-c3cnn(C)c3)c(C)c(=O)c2c1 ; Cc1cc([C@@H](C)Oc2ccc(Cl)nc2-c2noc(=O)[nH]2)c2oc(-c3cnc4cccnc4c3)c(C)c(=O)c2c1 ; Cc1cc([C@@H](C)Oc2ccc(Cl)nc2-c2noc(=O)[nH]2)c2oc(-c3cnc4c(c3)OCCN4C)c(C)c(=O)c2c1 ; Cc1cc([C@@H](C)Oc2ccc(Cl)nc2-c2noc(=O)[nH]2)c2oc(-c3ccc4c(c3)OCCN4C)c(C)c(=O)c2c1 ; Cc1cc([C@@H](C)Oc2ccc(Cl)nc2-c2noc(=O)[nH]2)c2oc(-c3cnc4c(c3)OCCO4)c(C)c(=O)c2c1]

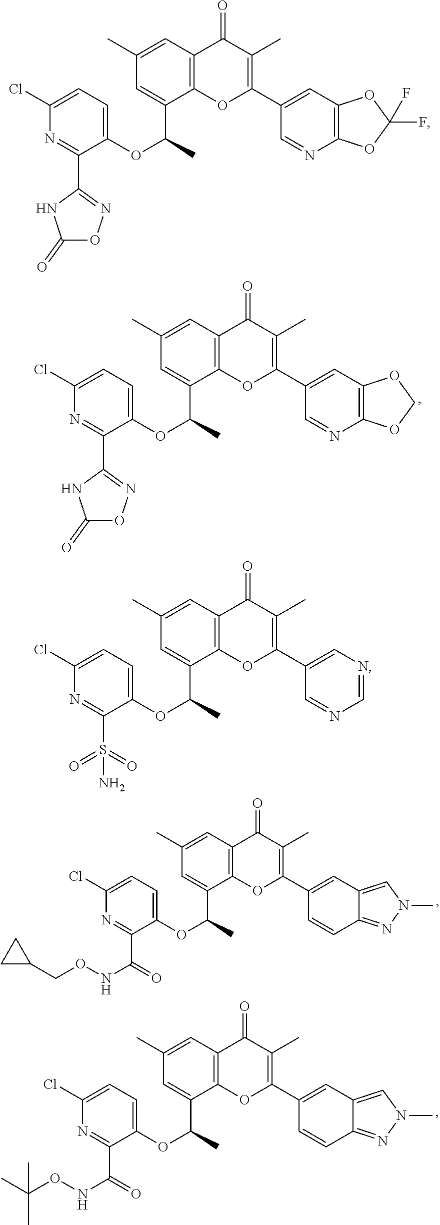

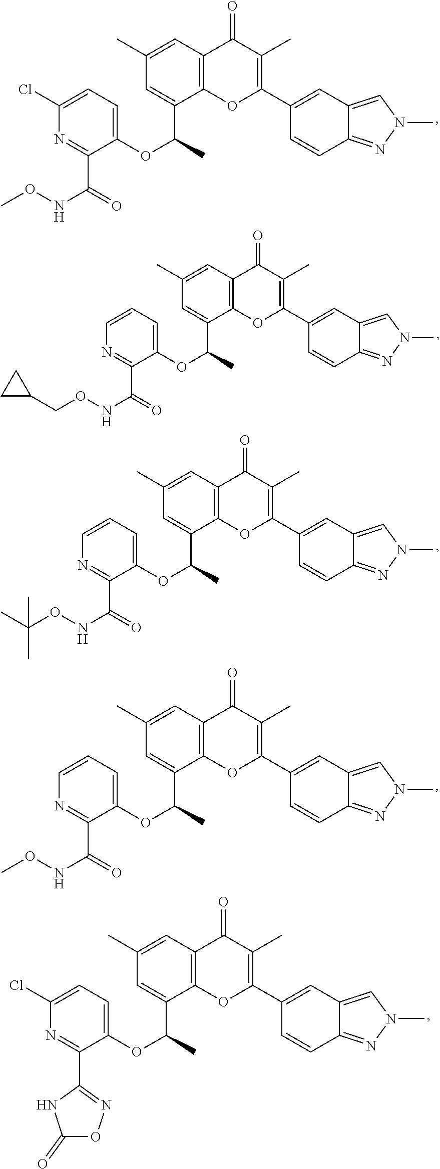

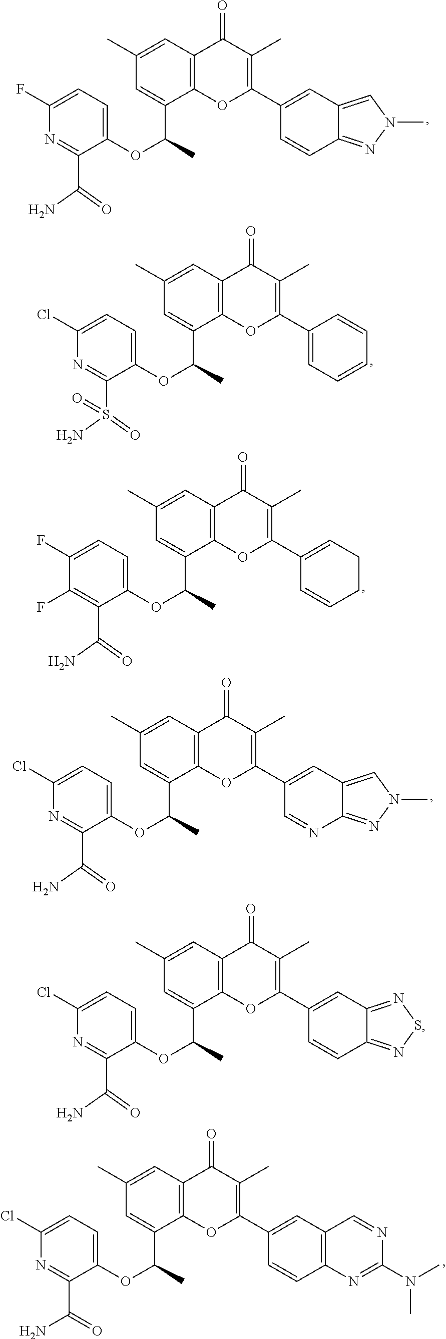 [SMILES: Cc1cc([C@@H](C)Oc2ccc(F)nc2C(N)=O)c2oc(-c3ccc4nn(C)cc4c3)c(C)c(=O)c2c1 ; Cc1cc([C@@H](C)Oc2ccc(Cl)nc2S(N)(=O)=O)c2oc(-c3ccccc3)c(C)c(=O)c2c1 ; Cc1cc([C@@H](C)Oc2ccc(F)c(F)c2C(N)=O)c2oc(C3=CCCC=C3)c(C)c(=O)c2c1 ; Cc1cc([C@@H](C)Oc2ccc(Cl)nc2C(N)=O)c2oc(-c3cnc4nn(C)cc4c3)c(C)c(=O)c2c1 ; Cc1cc([C@@H](C)Oc2ccc(Cl)nc2C(N)=O)c2oc(-c3ccc4nsnc4c3)c(C)c(=O)c2c1 ; Cc1cc([C@@H](C)Oc2ccc(Cl)nc2C(N)=O)c2oc(-c3ccc4nc(N(C)C)ncc4c3)c(C)c(=O)c2c1]

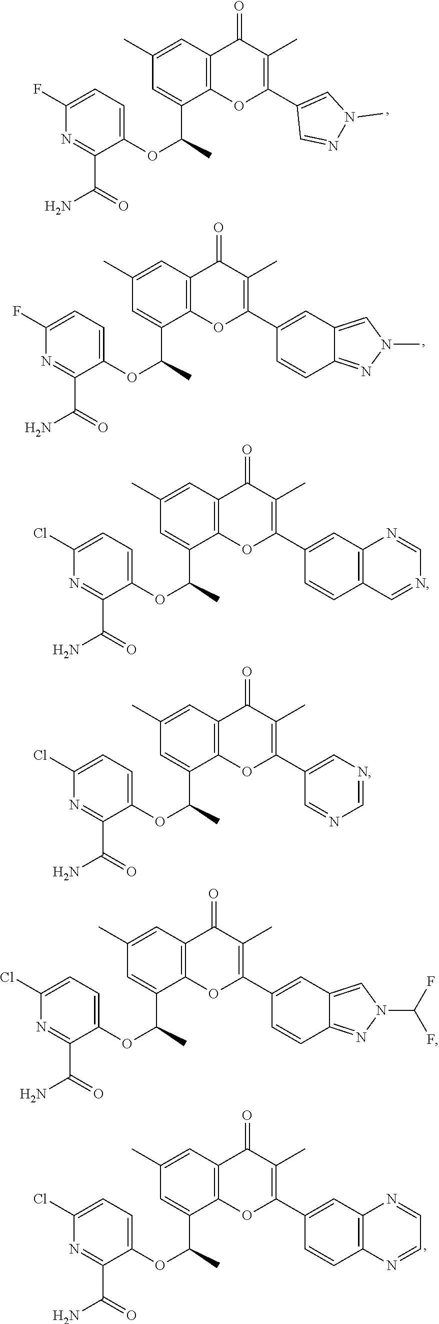 [SMILES: Cc1cc([C@@H](C)Oc2ccc(F)nc2C(N)=O)c2oc(-c3cnn(C)c3)c(C)c(=O)c2c1 ; Cc1cc([C@@H](C)Oc2ccc(F)nc2C(N)=O)c2oc(-c3ccc4nn(C)cc4c3)c(C)c(=O)c2c1 ; Cc1cc([C@@H](C)Oc2ccc(Cl)nc2C(N)=O)c2oc(-c3ccc4cncnc4c3)c(C)c(=O)c2c1 ; Cc1cc([C@@H](C)Oc2ccc(Cl)nc2C(N)=O)c2oc(-c3cncnc3)c(C)c(=O)c2c1 ; Cc1cc([C@@H](C)Oc2ccc(Cl)nc2C(N)=O)c2oc(-c3ccc4nn(C(F)F)cc4c3)c(C)c(=O)c2c1 ; Cc1cc([C@@H](C)Oc2ccc(Cl)nc2C(N)=O)c2oc(-c3ccc4nccnc4c3)c(C)c(=O)c2c1]

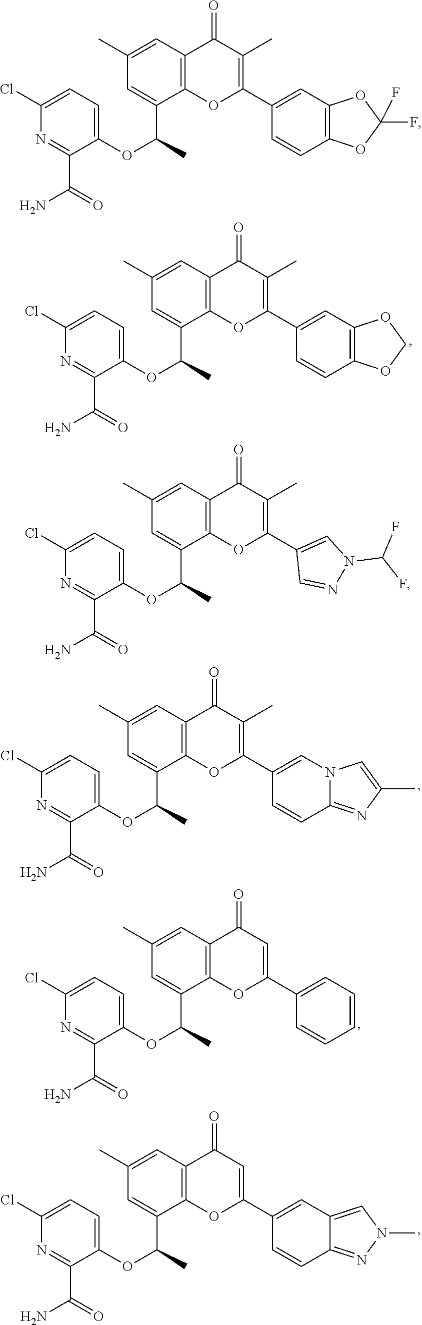 [SMILES: Cc1cc([C@@H](C)Oc2ccc(Cl)nc2C(N)=O)c2oc(-c3ccc4c(c3)OC(F)(F)O4)c(C)c(=O)c2c1 ; Cc1cc([C@@H](C)Oc2ccc(Cl)nc2C(N)=O)c2oc(-c3ccc4c(c3)OCO4)c(C)c(=O)c2c1 ; Cc1cc([C@@H](C)Oc2ccc(Cl)nc2C(N)=O)c2oc(-c3cnn(C(F)F)c3)c(C)c(=O)c2c1 ; Cc1cc([C@@H](C)Oc2ccc(Cl)nc2C(N)=O)c2oc(-c3ccc4nc(C)cn4c3)c(C)c(=O)c2c1 ; Cc1cc([C@@H](C)Oc2ccc(Cl)nc2C(N)=O)c2oc(-c3ccccc3)cc(=O)c2c1 ; Cc1cc([C@@H](C)Oc2ccc(Cl)nc2C(N)=O)c2oc(-c3ccc4nn(C)cc4c3)cc(=O)c2c1]

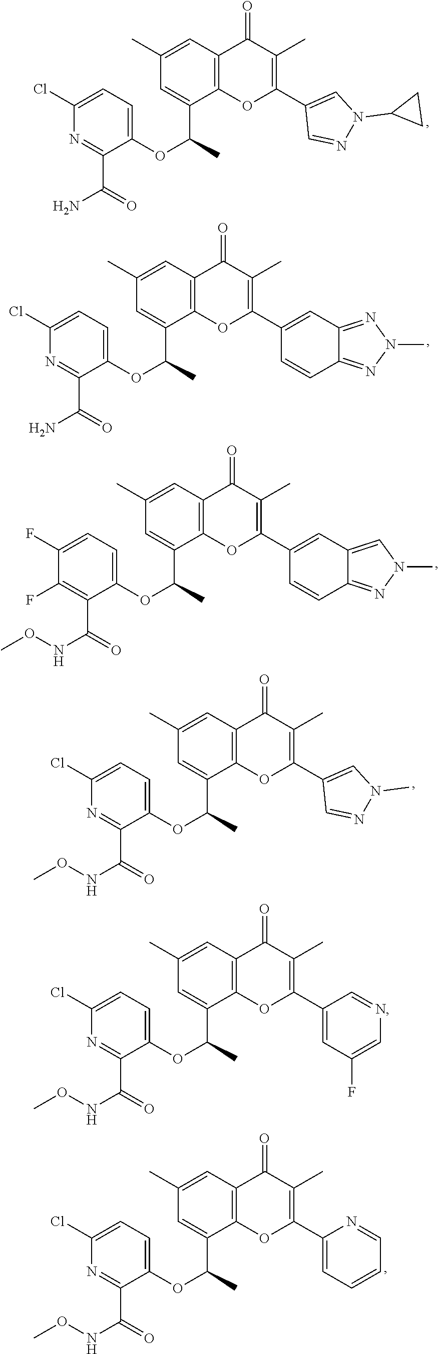

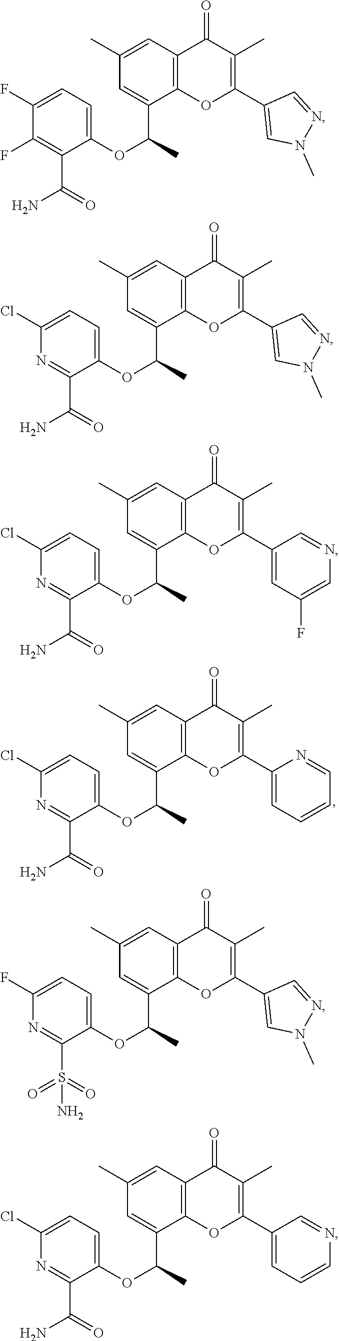 [SMILES: Cc1cc([C@@H](C)Oc2ccc(F)c(F)c2C(N)=O)c2oc(-c3cnn(C)c3)c(C)c(=O)c2c1 ; Cc1cc([C@@H](C)Oc2ccc(Cl)nc2C(N)=O)c2oc(-c3cnn(C)c3)c(C)c(=O)c2c1 ; Cc1cc([C@@H](C)Oc2ccc(Cl)nc2C(N)=O)c2oc(-c3cncc(F)c3)c(C)c(=O)c2c1 ; Cc1cc([C@@H](C)Oc2ccc(Cl)nc2C(N)=O)c2oc(-c3ccccn3)c(C)c(=O)c2c1 ; Cc1cc([C@@H](C)Oc2ccc(F)nc2S(N)(=O)=O)c2oc(-c3cnn(C)c3)c(C)c(=O)c2c1 ; Cc1cc([C@@H](C)Oc2ccc(Cl)nc2C(N)=O)c2oc(-c3cccnc3)c(C)c(=O)c2c1]

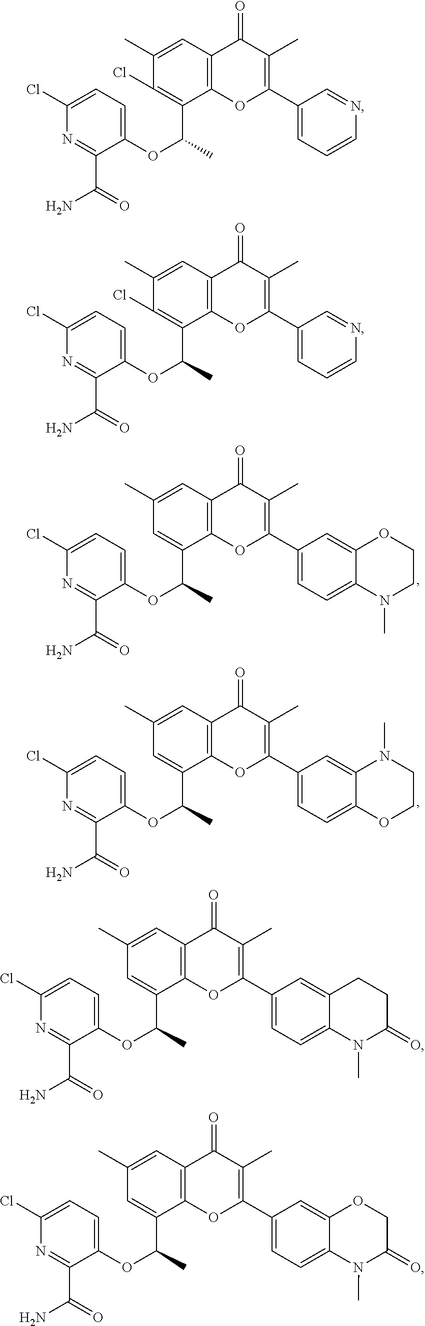 [SMILES: Cc1cc2c(=O)c(C)c(-c3cccnc3)oc2c([C@H](C)Oc2ccc(Cl)nc2C(N)=O)c1Cl ; Cc1cc2c(=O)c(C)c(-c3cccnc3)oc2c([C@@H](C)Oc2ccc(Cl)nc2C(N)=O)c1Cl ; Cc1cc([C@@H](C)Oc2ccc(Cl)nc2C(N)=O)c2oc(-c3ccc4c(c3)OCCN4C)c(C)c(=O)c2c1 ; Cc1cc([C@@H](C)Oc2ccc(Cl)nc2C(N)=O)c2oc(-c3ccc4c(c3)N(C)CCO4)c(C)c(=O)c2c1 ; Cc1cc([C@@H](C)Oc2ccc(Cl)nc2C(N)=O)c2oc(-c3ccc4c(c3)CCC(=O)N4C)c(C)c(=O)c2c1 ; Cc1cc([C@@H](C)Oc2ccc(Cl)nc2C(N)=O)c2oc(-c3ccc4c(c3)OCC(=O)N4C)c(C)c(=O)c2c1]

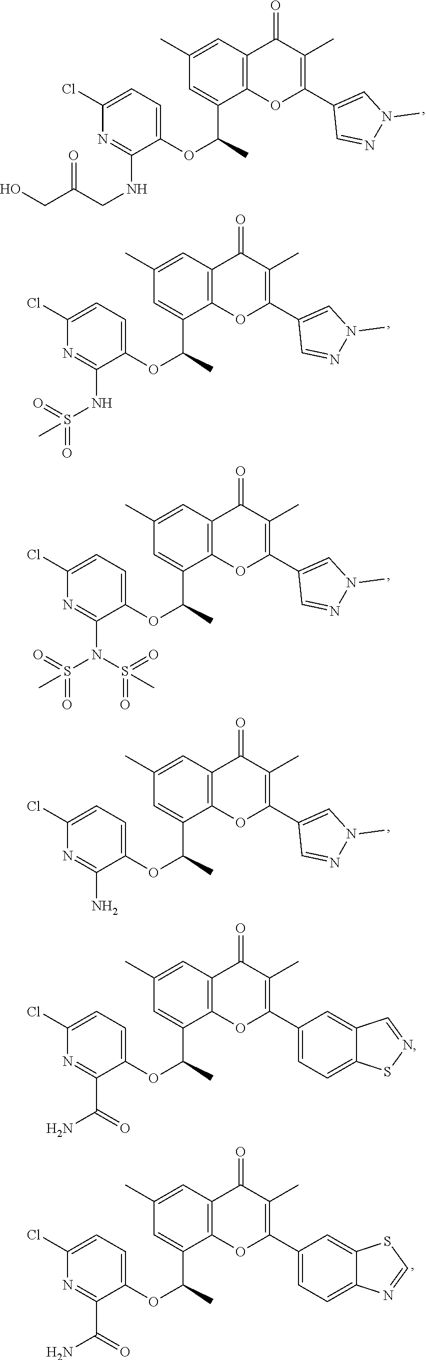 [SMILES: Cc1cc([C@@H](C)Oc2ccc(Cl)nc2NCC(=O)CO)c2oc(-c3cnn(C)c3)c(C)c(=O)c2c1 ; Cc1cc([C@@H](C)Oc2ccc(Cl)nc2NS(C)(=O)=O)c2oc(-c3cnn(C)c3)c(C)c(=O)c2c1 ; Cc1cc([C@@H](C)Oc2ccc(Cl)nc2N(S(C)(=O)=O)S(C)(=O)=O)c2oc(-c3cnn(C)c3)c(C)c(=O)c2c1 ; Cc1cc([C@@H](C)Oc2ccc(Cl)nc2N)c2oc(-c3cnn(C)c3)c(C)c(=O)c2c1 ; Cc1cc([C@@H](C)Oc2ccc(Cl)nc2C(N)=O)c2oc(-c3ccc4sncc4c3)c(C)c(=O)c2c1 ; Cc1cc([C@@H](C)Oc2ccc(Cl)nc2C(N)=O)c2oc(-c3ccc4ncsc4c3)c(C)c(=O)c2c1]

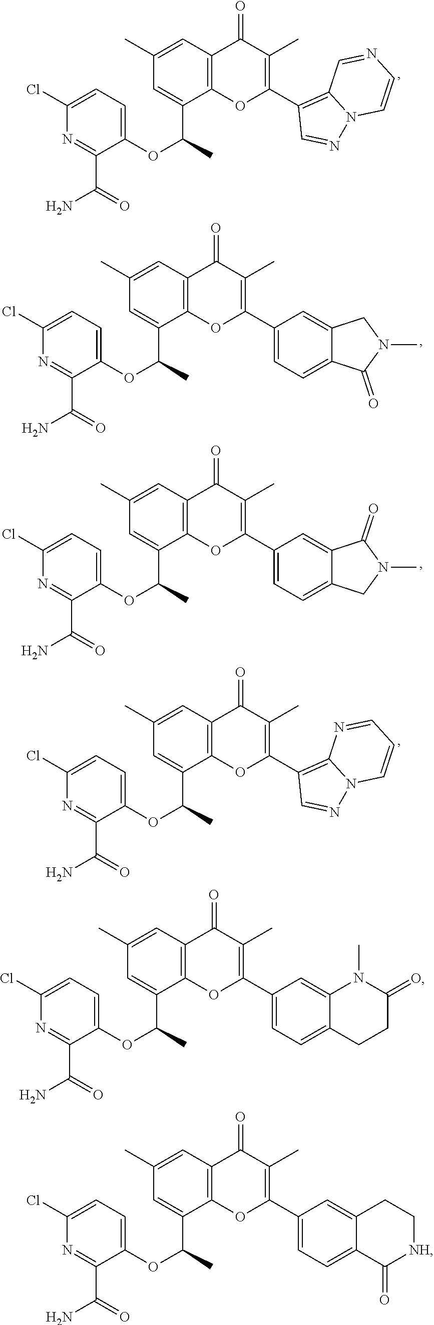 [SMILES: Cc1cc([C@@H](C)Oc2ccc(Cl)nc2C(N)=O)c2oc(-c3cnn4ccncc34)c(C)c(=O)c2c1 ; Cc1cc([C@@H](C)Oc2ccc(Cl)nc2C(N)=O)c2oc(-c3ccc4c(c3)CN(C)C4=O)c(C)c(=O)c2c1 ; Cc1cc([C@@H](C)Oc2ccc(Cl)nc2C(N)=O)c2oc(-c3ccc4c(c3)C(=O)N(C)C4)c(C)c(=O)c2c1 ; Cc1cc([C@@H](C)Oc2ccc(Cl)nc2C(N)=O)c2oc(-c3cnn4cccnc34)c(C)c(=O)c2c1 ; Cc1cc([C@@H](C)Oc2ccc(Cl)nc2C(N)=O)c2oc(-c3ccc4c(c3)N(C)C(=O)CC4)c(C)c(=O)c2c1 ; Cc1cc([C@@H](C)Oc2ccc(Cl)nc2C(N)=O)c2oc(-c3ccc4c(c3)CCNC4=O)c(C)c(=O)c2c1]

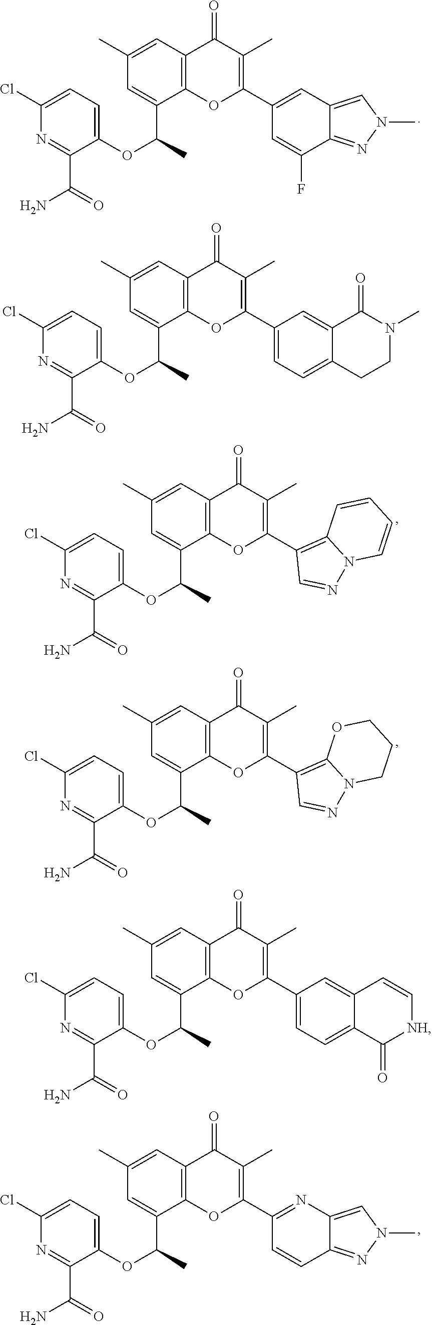 [SMILES: Cc1cc([C@@H](C)Oc2ccc(Cl)nc2C(N)=O)c2oc(-c3cc(F)c4nn(C)cc4c3)c(C)c(=O)c2c1 ; Cc1cc([C@@H](C)Oc2ccc(Cl)nc2C(N)=O)c2oc(-c3ccc4c(c3)C(=O)N(C)CC4)c(C)c(=O)c2c1 ; Cc1cc([C@@H](C)Oc2ccc(Cl)nc2C(N)=O)c2oc(-c3cnn4ccccc34)c(C)c(=O)c2c1 ; Cc1cc([C@@H](C)Oc2ccc(Cl)nc2C(N)=O)c2oc(-c3cnn4c3OCCC4)c(C)c(=O)c2c1 ; Cc1cc([C@@H](C)Oc2ccc(Cl)nc2C(N)=O)c2oc(-c3ccc4c(=O)[nH]ccc4c3)c(C)c(=O)c2c1 ; Cc1cc([C@@H](C)Oc2ccc(Cl)nc2C(N)=O)c2oc(-c3ccc4nn(C)cc4n3)c(C)c(=O)c2c1]

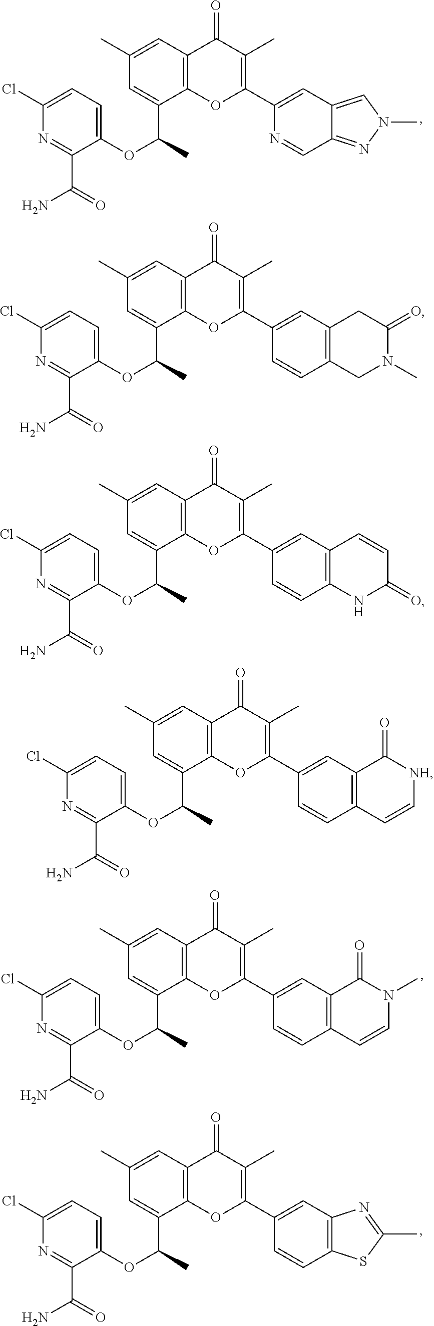 [SMILES: Cc1cc([C@@H](C)Oc2ccc(Cl)nc2C(N)=O)c2oc(-c3cc4cn(C)nc4cn3)c(C)c(=O)c2c1 ; Cc1cc([C@@H](C)Oc2ccc(Cl)nc2C(N)=O)c2oc(-c3ccc4c(c3)CC(=O)N(C)C4)c(C)c(=O)c2c1 ; Cc1cc([C@@H](C)Oc2ccc(Cl)nc2C(N)=O)c2oc(-c3ccc4[nH]c(=O)ccc4c3)c(C)c(=O)c2c1 ; Cc1cc([C@@H](C)Oc2ccc(Cl)nc2C(N)=O)c2oc(-c3ccc4cc[nH]c(=O)c4c3)c(C)c(=O)c2c1 ; Cc1cc([C@@H](C)Oc2ccc(Cl)nc2C(N)=O)c2oc(-c3ccc4ccn(C)c(=O)c4c3)c(C)c(=O)c2c1 ; Cc1cc([C@@H](C)Oc2ccc(Cl)nc2C(N)=O)c2oc(-c3ccc4sc(C)nc4c3)c(C)c(=O)c2c1]

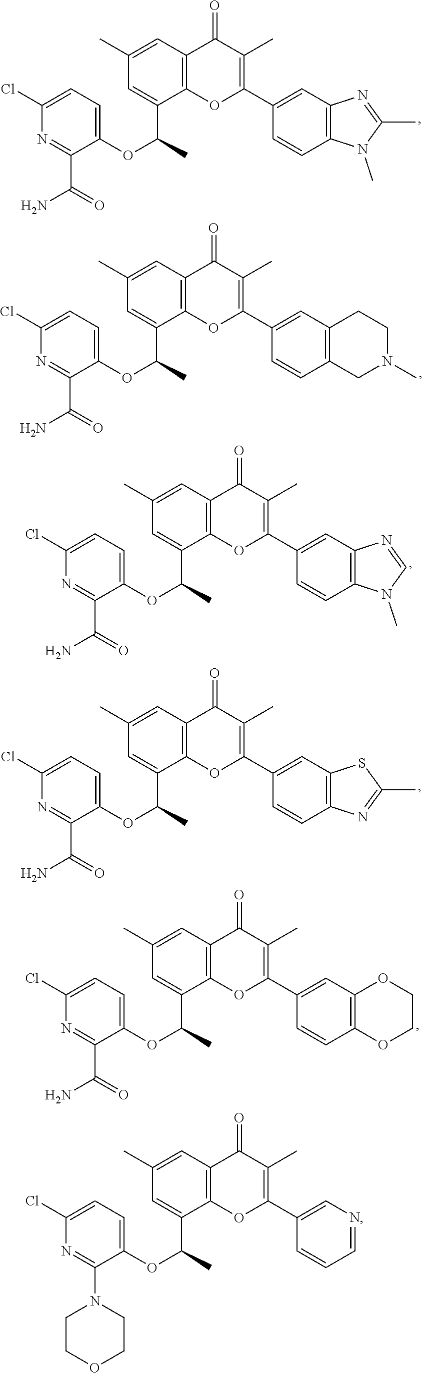 [SMILES: Cc1cc([C@@H](C)Oc2ccc(Cl)nc2C(N)=O)c2oc(-c3ccc4c(c3)nc(C)n4C)c(C)c(=O)c2c1 ; Cc1cc([C@@H](C)Oc2ccc(Cl)nc2C(N)=O)c2oc(-c3ccc4c(c3)CCN(C)C4)c(C)c(=O)c2c1 ; Cc1cc([C@@H](C)Oc2ccc(Cl)nc2C(N)=O)c2oc(-c3ccc4c(c3)ncn4C)c(C)c(=O)c2c1 ; Cc1cc([C@@H](C)Oc2ccc(Cl)nc2C(N)=O)c2oc(-c3ccc4nc(C)sc4c3)c(C)c(=O)c2c1 ; Cc1cc([C@@H](C)Oc2ccc(Cl)nc2C(N)=O)c2oc(-c3ccc4c(c3)OCCO4)c(C)c(=O)c2c1 ; Cc1cc([C@@H](C)Oc2ccc(Cl)nc2N2CCOCC2)c2oc(-c3cccnc3)c(C)c(=O)c2c1]

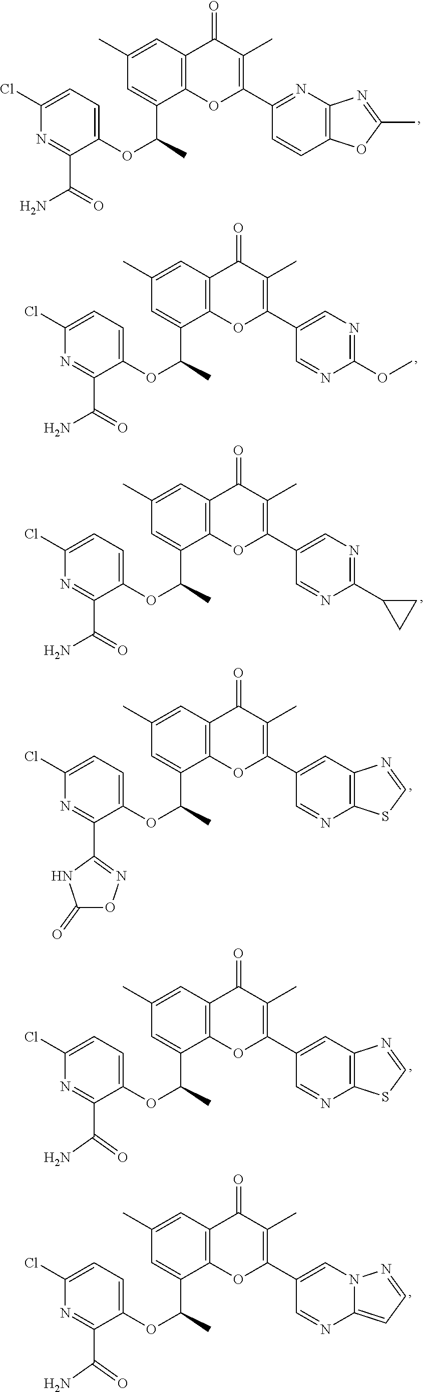 [SMILES: Cc1cc([C@@H](C)Oc2ccc(Cl)nc2C(N)=O)c2oc(-c3ccc4oc(C)nc4n3)c(C)c(=O)c2c1 ; COc1ncc(-c2oc3c([C@@H](C)Oc4ccc(Cl)nc4C(N)=O)cc(C)cc3c(=O)c2C)cn1 ; Cc1cc([C@@H](C)Oc2ccc(Cl)nc2C(N)=O)c2oc(-c3cnc(C4CC4)nc3)c(C)c(=O)c2c1 ; Cc1cc([C@@H](C)Oc2ccc(Cl)nc2-c2noc(=O)[nH]2)c2oc(-c3cnc4scnc4c3)c(C)c(=O)c2c1 ; Cc1cc([C@@H](C)Oc2ccc(Cl)nc2C(N)=O)c2oc(-c3cnc4scnc4c3)c(C)c(=O)c2c1 ; Cc1cc([C@@H](C)Oc2ccc(Cl)nc2C(N)=O)c2oc(-c3cnc4ccnn4c3)c(C)c(=O)c2c1]

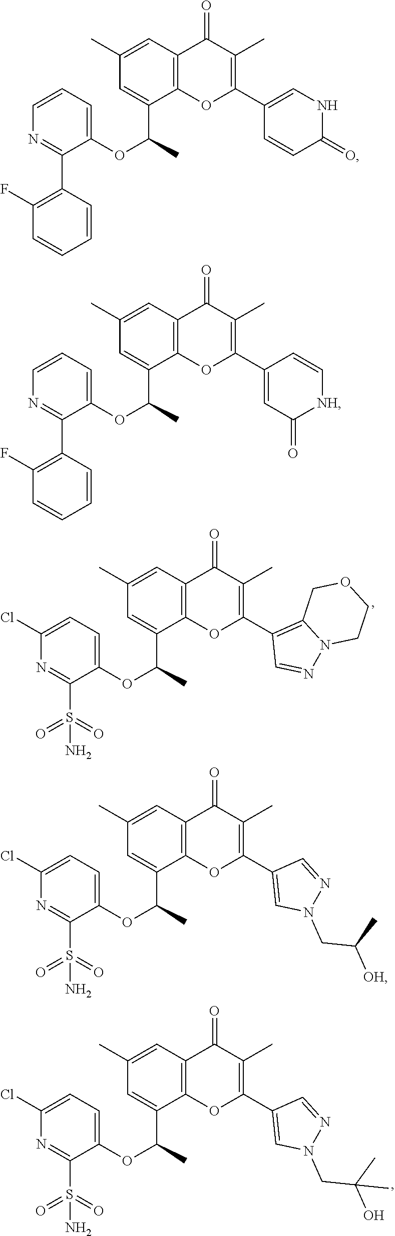 [SMILES: Cc1cc([C@@H](C)Oc2cccnc2-c2ccccc2F)c2oc(-c3ccc(=O)[nH]c3)c(C)c(=O)c2c1 ; Cc1cc([C@@H](C)Oc2cccnc2-c2ccccc2F)c2oc(-c3cc[nH]c(=O)c3)c(C)c(=O)c2c1 ; Cc1cc([C@@H](C)Oc2ccc(Cl)nc2S(N)(=O)=O)c2oc(-c3cnn4c3COCC4)c(C)c(=O)c2c1 ; Cc1cc([C@@H](C)Oc2ccc(Cl)nc2S(N)(=O)=O)c2oc(-c3cnn(C[C@@H](C)O)c3)c(C)c(=O)c2c1 ; Cc1cc([C@@H](C)Oc2ccc(Cl)nc2S(N)(=O)=O)c2oc(-c3cnn(CC(C)(C)O)c3)c(C)c(=O)c2c1]

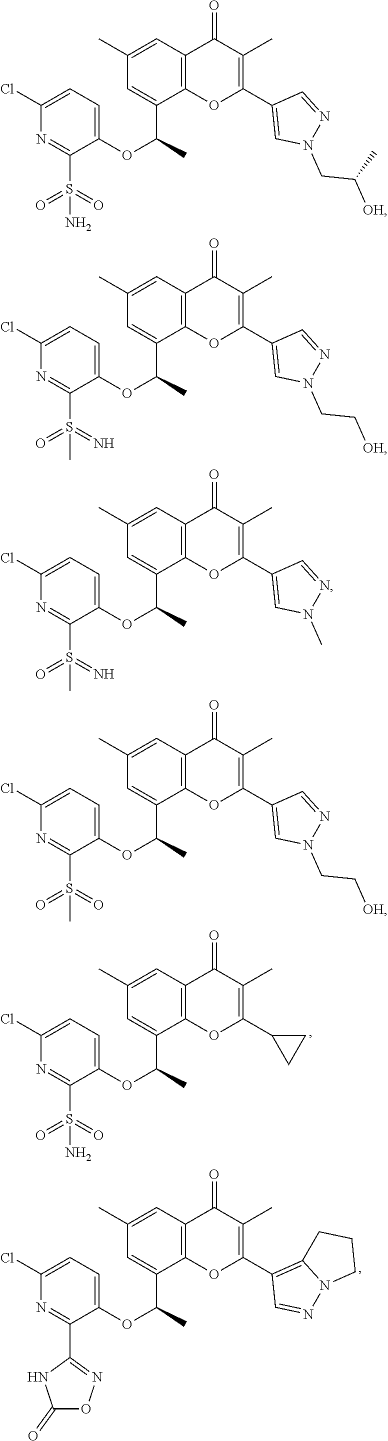 [SMILES: Cc1cc([C@@H](C)Oc2ccc(Cl)nc2S(N)(=O)=O)c2oc(-c3cnn(C[C@H](C)O)c3)c(C)c(=O)c2c1 ; Cc1cc([C@@H](C)Oc2ccc(Cl)nc2S(C)(=N)=O)c2oc(-c3cnn(CCO)c3)c(C)c(=O)c2c1 ; Cc1cc([C@@H](C)Oc2ccc(Cl)nc2S(C)(=N)=O)c2oc(-c3cnn(C)c3)c(C)c(=O)c2c1 ; Cc1cc([C@@H](C)Oc2ccc(Cl)nc2S(C)(=O)=O)c2oc(-c3cnn(CCO)c3)c(C)c(=O)c2c1 ; Cc1cc([C@@H](C)Oc2ccc(Cl)nc2S(N)(=O)=O)c2oc(C3CC3)c(C)c(=O)c2c1 ; Cc1cc([C@@H](C)Oc2ccc(Cl)nc2-c2noc(=O)[nH]2)c2oc(-c3cnn4c3CCC4)c(C)c(=O)c2c1]

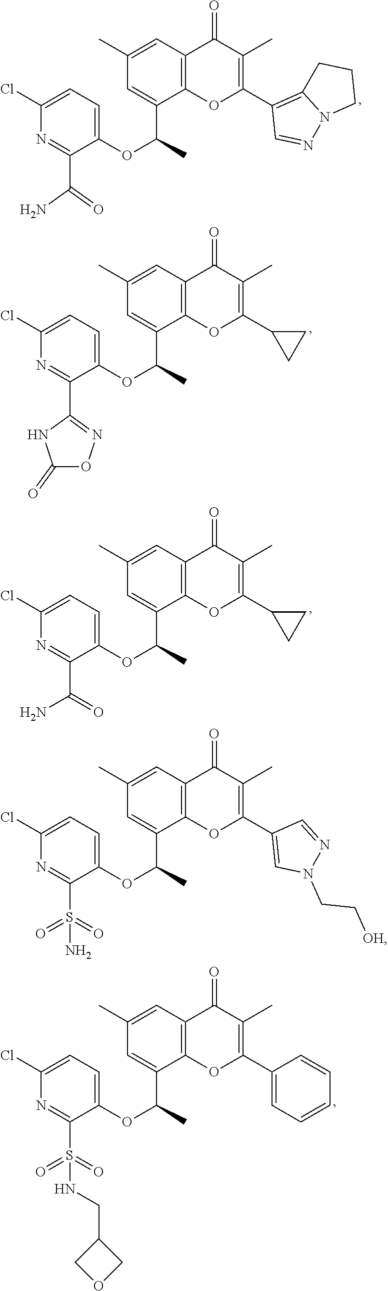 [SMILES: Cc1cc([C@@H](C)Oc2ccc(Cl)nc2C(N)=O)c2oc(-c3cnn4c3CCC4)c(C)c(=O)c2c1 ; Cc1cc([C@@H](C)Oc2ccc(Cl)nc2-c2noc(=O)[nH]2)c2oc(C3CC3)c(C)c(=O)c2c1 ; Cc1cc([C@@H](C)Oc2ccc(Cl)nc2C(N)=O)c2oc(C3CC3)c(C)c(=O)c2c1 ; Cc1cc([C@@H](C)Oc2ccc(Cl)nc2S(N)(=O)=O)c2oc(-c3cnn(CCO)c3)c(C)c(=O)c2c1 ; Cc1cc([C@@H](C)Oc2ccc(Cl)nc2S(=O)(=O)NCC2COC2)c2oc(-c3ccccc3)c(C)c(=O)c2c1]

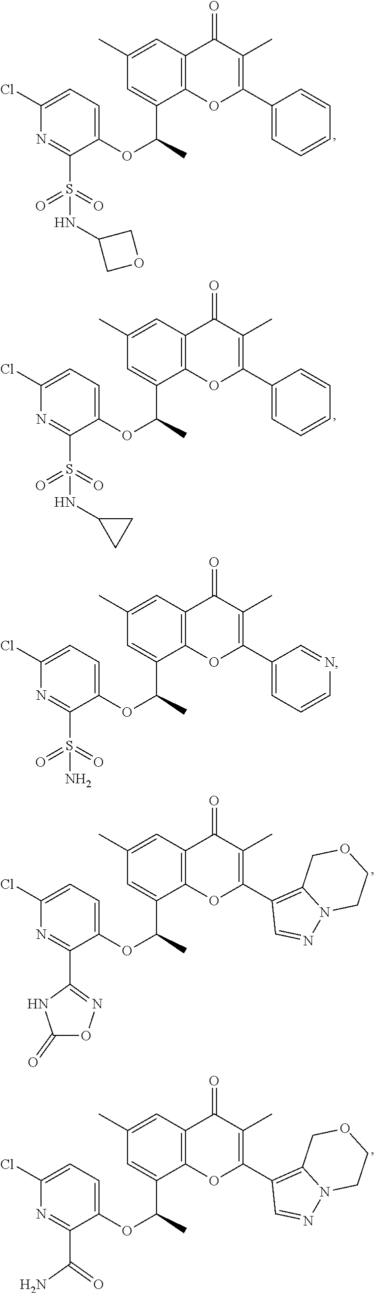 [SMILES: Cc1cc([C@@H](C)Oc2ccc(Cl)nc2S(=O)(=O)NC2COC2)c2oc(-c3ccccc3)c(C)c(=O)c2c1 ; Cc1cc([C@@H](C)Oc2ccc(Cl)nc2S(=O)(=O)NC2CC2)c2oc(-c3ccccc3)c(C)c(=O)c2c1 ; Cc1cc([C@@H](C)Oc2ccc(Cl)nc2S(N)(=O)=O)c2oc(-c3cccnc3)c(C)c(=O)c2c1 ; Cc1cc([C@@H](C)Oc2ccc(Cl)nc2-c2noc(=O)[nH]2)c2oc(-c3cnn4c3COCC4)c(C)c(=O)c2c1 ; Cc1cc([C@@H](C)Oc2ccc(Cl)nc2C(N)=O)c2oc(-c3cnn4c3COCC4)c(C)c(=O)c2c1]

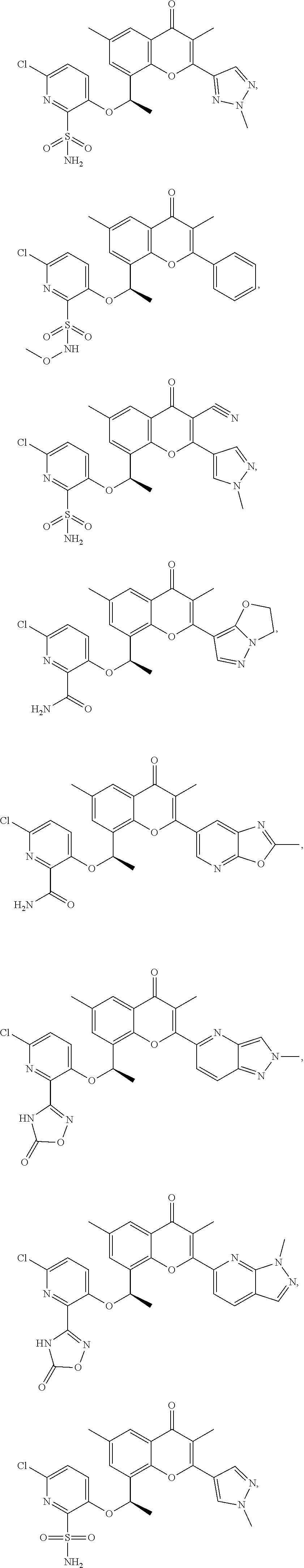

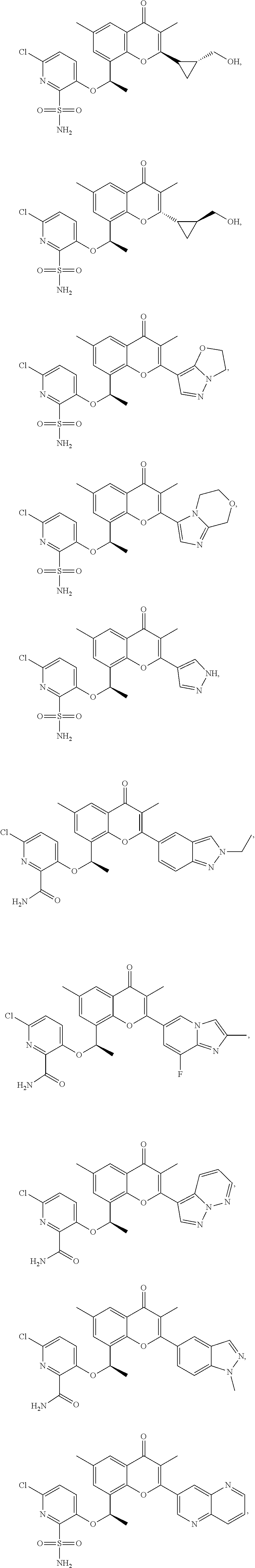

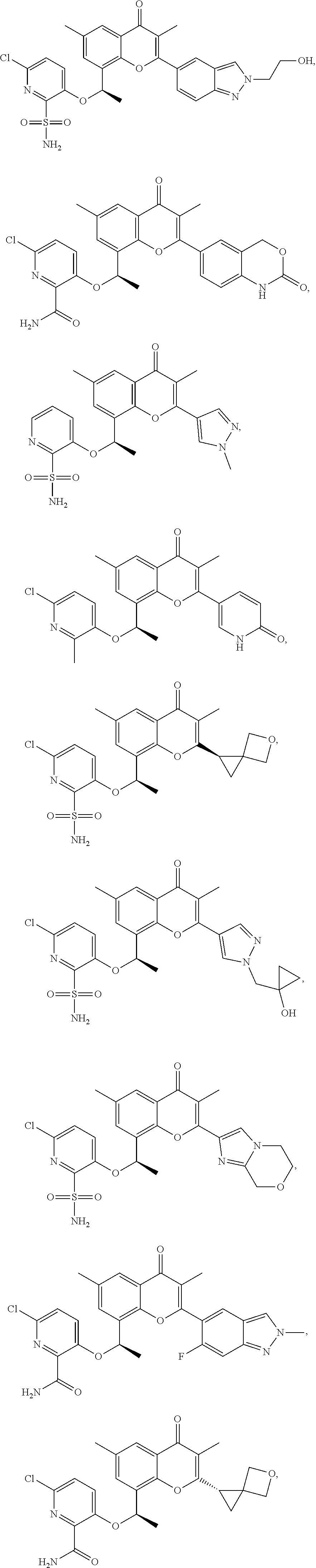

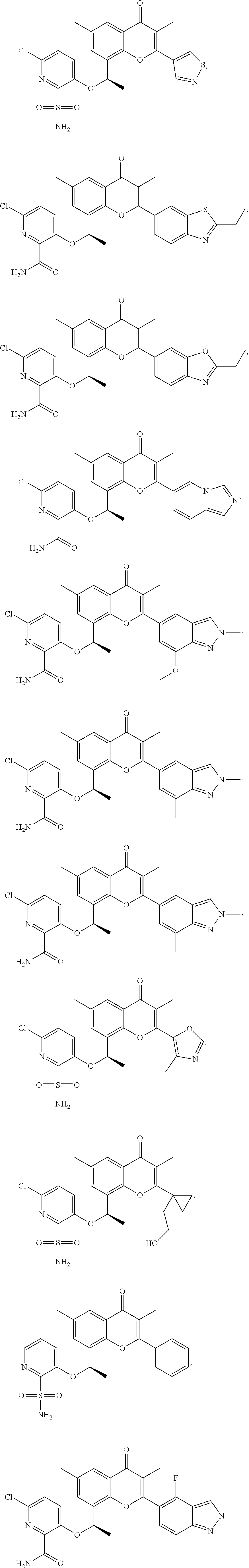

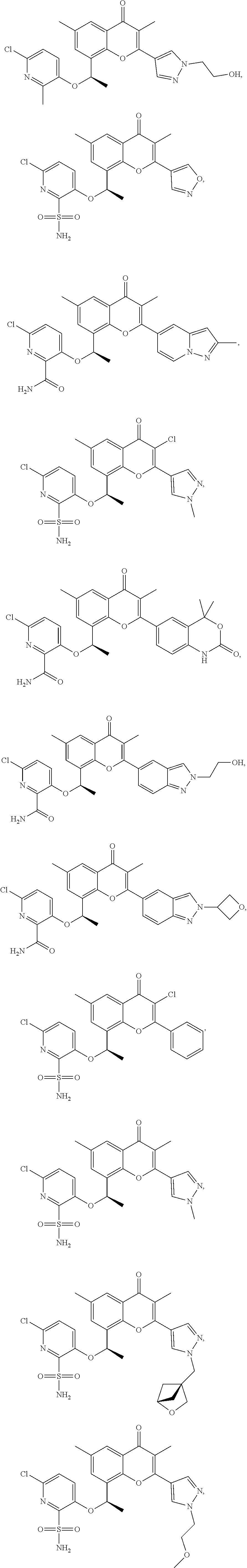

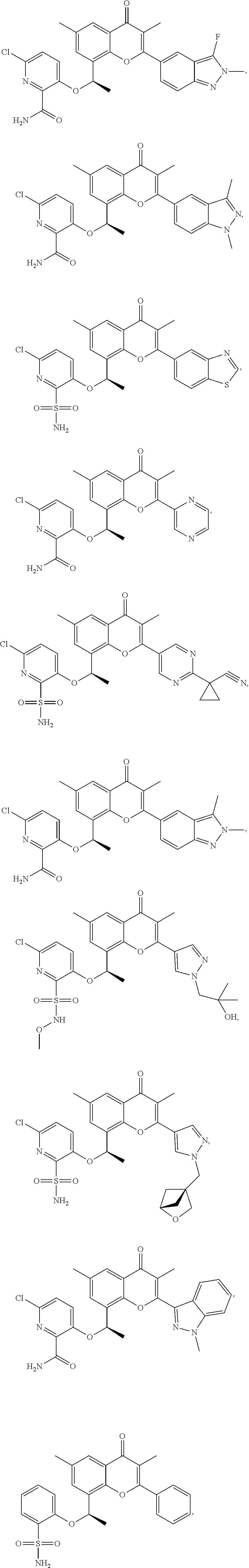

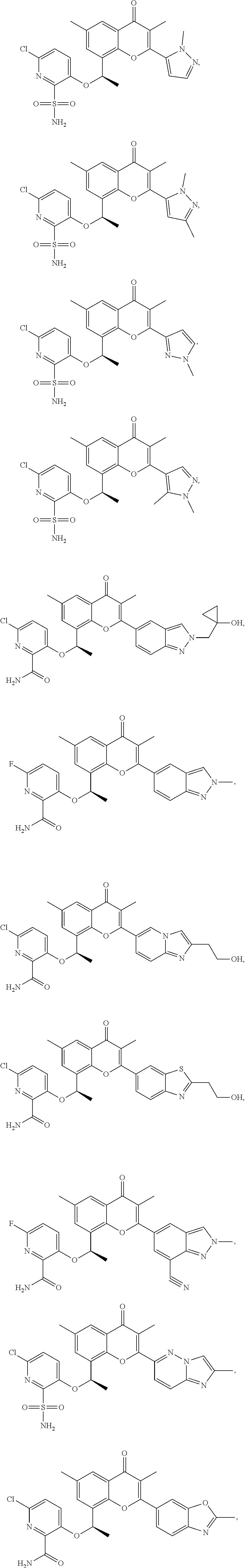

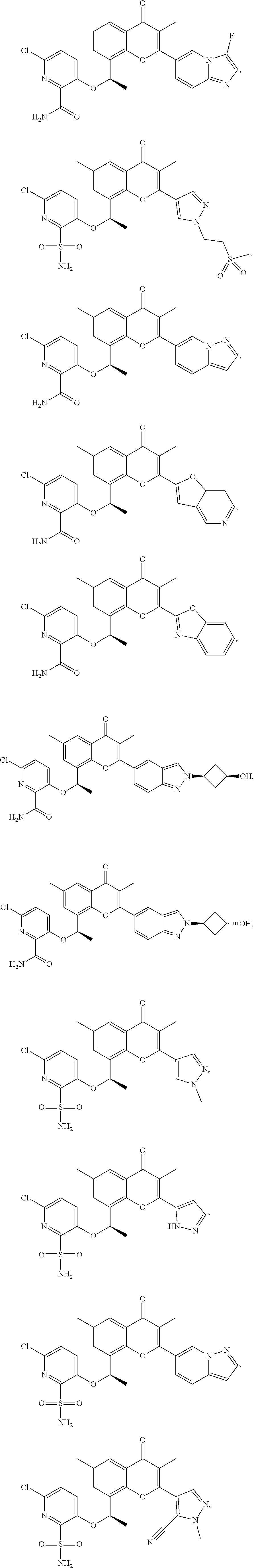

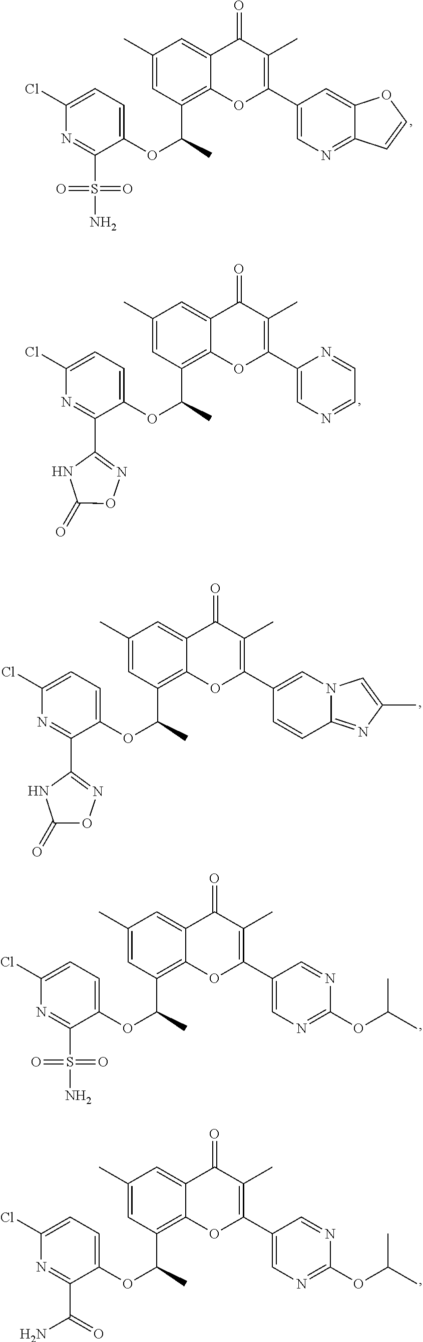 [SMILES: Cc1cc([C@@H](C)Oc2ccc(Cl)nc2S(N)(=O)=O)c2oc(-c3cnc4ccoc4c3)c(C)c(=O)c2c1 ; Cc1cc([C@@H](C)Oc2ccc(Cl)nc2-c2noc(=O)[nH]2)c2oc(-c3cnccn3)c(C)c(=O)c2c1 ; Cc1cc([C@@H](C)Oc2ccc(Cl)nc2-c2noc(=O)[nH]2)c2oc(-c3ccc4nc(C)cn4c3)c(C)c(=O)c2c1 ; Cc1cc([C@@H](C)Oc2ccc(Cl)nc2S(N)(=O)=O)c2oc(-c3cnc(OC(C)C)nc3)c(C)c(=O)c2c1 ; Cc1cc([C@@H](C)Oc2ccc(Cl)nc2C(N)=O)c2oc(-c3cnc(OC(C)C)nc3)c(C)c(=O)c2c1]

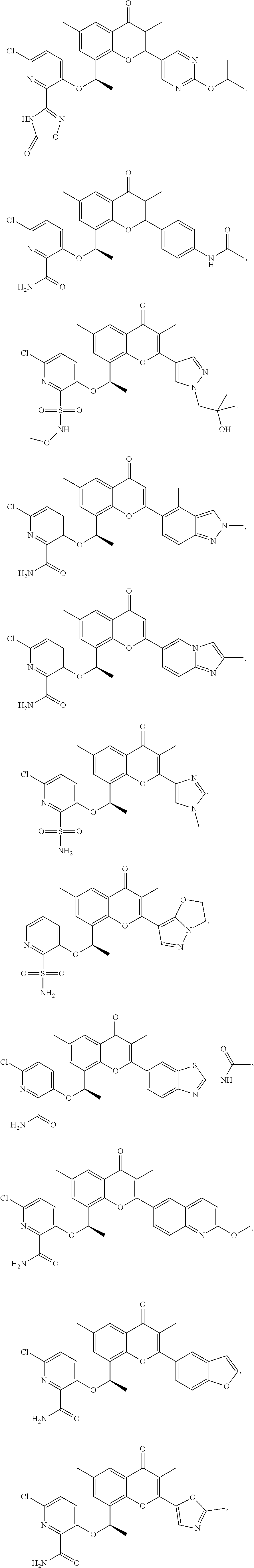

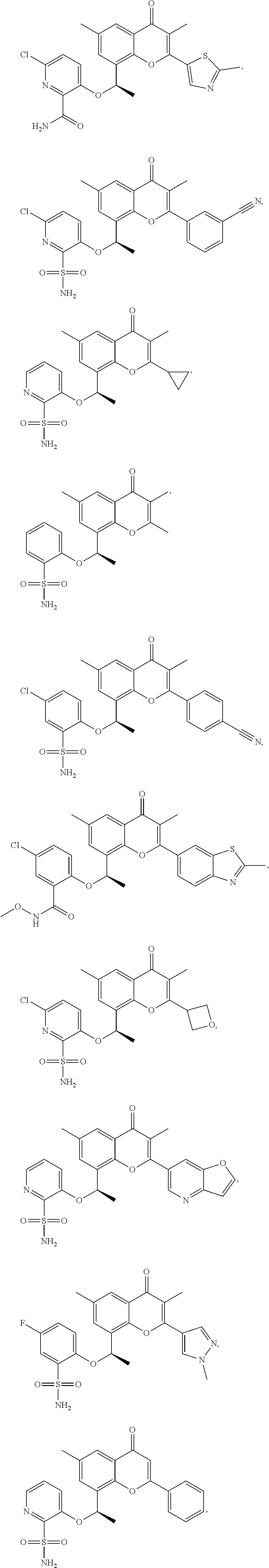

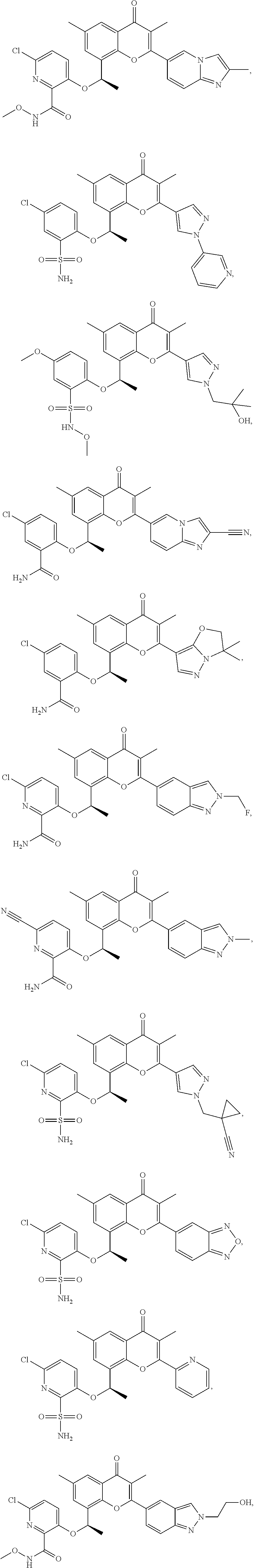

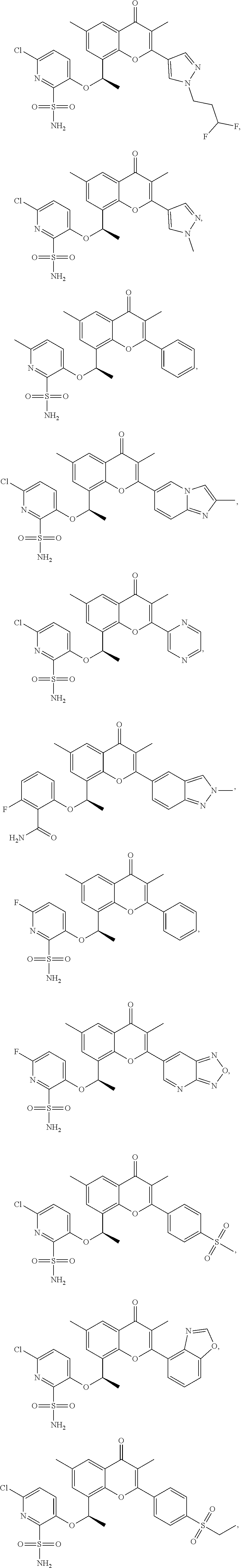

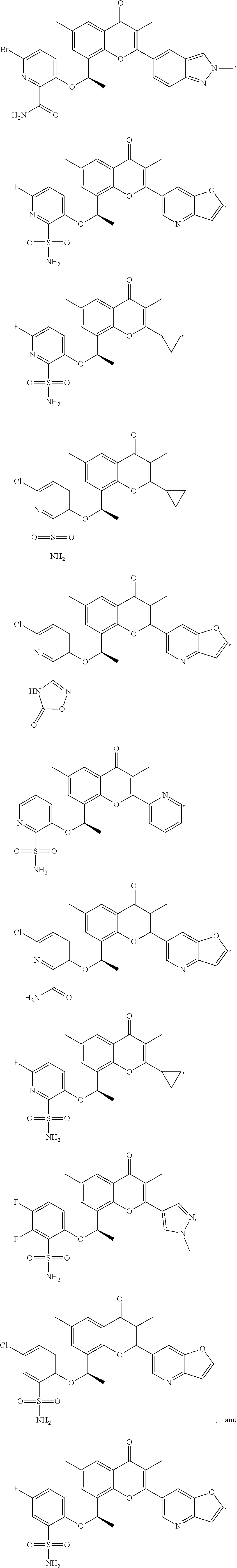

31. A pharmaceutical composition comprising a compound as defined in any one of aspects 1-30, or a pharmaceutically acceptable salt thereof, and a pharmaceutically acceptable carrier.
32. A method of treating a disease or disorder associated with modulation of phosphoinositide 3-kinase (PI3K), comprising administering to a patient in need thereof, a therapeutically effective amount of a compound as defined in any one of aspects 1-30, or a pharmaceutically acceptable salt thereof, or a pharmaceutical composition as defined in aspect 31.
33. The method as defined in aspect 32, wherein the PI3K is PI3Kα.
34. The method as defined in aspect 32 or aspect 33, wherein the PI3K associated with the disease or disorder has a H1047R mutation, an E575K mutation, or both of a H1047R mutation and an E575K mutation.
35. The method as defined in any one of aspects 32-34, wherein the disease or disorder is a cancer.
36. The method as defined in aspect 35, wherein the cancer is endometrial cancer, gastric cancer, leukemia, lymphoma, sarcoma, colorectal cancer, lung cancer, ovarian cancer, skin cancer, head and neck cancer, breast cancer, brain cancer, or prostate cancer.
37. The method as defined in aspect 35, wherein the cancer is breast cancer.
38. The method as defined in aspect 35, wherein the cancer is hormone receptor-positive (HR+), human epidermal growth factor receptor 2-negative (HER2−) advanced or metastatic breast cancer.
39. The method as defined in in any one of aspects 32-34, wherein the disorder is CLOVES syndrome, or PROS.
40. A method of inhibiting phosphoinositide 3-kinase (PI3K), comprising administering to a patient in need thereof a therapeutically effective amount of a compound as defined in any one of aspects 1-30, or a pharmaceutically acceptable salt thereof, or a pharmaceutical composition as defined in aspect 31.
41. A method of treating cancer or a disorder, the method comprising administering to a patient in need thereof a therapeutically effective amount of a compound as defined in any one of aspects 1-30, or a pharmaceutically acceptable salt thereof, or a pharmaceutical composition as defined in aspect 31.
42. The method as defined in aspect 41, wherein the cancer is endometrial cancer, gastric cancer, leukemia, lymphoma, sarcoma, colorectal cancer, lung cancer, ovarian cancer, skin cancer, head and neck cancer, breast cancer, brain cancer, or prostate cancer.
43. The method as defined in aspect 41, wherein the cancer is breast cancer.
44. The method as defined in aspect 41, wherein the cancer is hormone receptor-positive (HR+), human epidermal growth factor receptor 2-negative (HER2−) advanced or metastatic breast cancer.
45. The method as defined in aspect 41, wherein the disorder is CLOVES syndrome or PROS.
46. The compound as defined in any one of aspects 1-30, or pharmaceutically acceptable salt thereof, for use in therapy.
47. The compound as defined in any one of aspects 1-30, or a pharmaceutically acceptable salt thereof, for use in the treatment of a disease or disorder associated with modulating PI3K.
48. The compound or a pharmaceutically acceptable salt thereof for use as defined in aspect 47, wherein the disease or disorder associated with modulating PI3K is a cancer.
49. The compound or a pharmaceutically acceptable salt thereof for use as defined in aspect 48, wherein the cancer is endometrial cancer, gastric cancer, leukemia, lymphoma, sarcoma, colorectal cancer, lung cancer, ovarian cancer, skin cancer, head and neck cancer, breast cancer, brain cancer, or prostate cancer.
50. The compound or a pharmaceutically acceptable salt thereof for use as defined in aspect 48, wherein the cancer is breast cancer.
51. The compound or a pharmaceutically acceptable salt thereof for use as defined in aspect 48, wherein the cancer is hormone receptor-positive (HR+), human epidermal growth factor receptor 2-negative (HER2−) advanced or metastatic breast cancer.
52. The compound or a pharmaceutically acceptable salt thereof for use as defined in aspect 47, wherein the disorder is CLOVES syndrome or PROS.
53. Use of a compound as defined in any one of aspects 1-30, or a pharmaceutically acceptable salt thereof, in the manufacture of a medicament for the treatment of a disease or disorder associated with modulating PI3K.
54. The use as defined in aspect 53, wherein the disease or disorder associated with modulating PI3K is a cancer.
55. The use as defined in aspect 54, wherein the cancer is endometrial cancer, gastric cancer, leukemia, lymphoma, sarcoma, colorectal cancer, lung cancer, ovarian cancer, skin cancer, head and neck cancer, breast cancer, brain cancer, or prostate cancer.
56. The use as defined in aspect 54, wherein the cancer is breast cancer.
57. The use as defined in aspect 54, wherein the cancer is hormone receptor-positive (HR+), human epidermal growth factor receptor 2-negative (HER2−) advanced or metastatic breast cancer.
58. The use as defined in aspect 53, wherein the disorder is CLOVES syndrome or PROS.

### Pharmaceutical Compositions

In some aspects, the present disclosure provides a pharmaceutical composition comprising a compound of Formula (I), (II), (III), or (IV) as an active ingredient. In some embodiments, the present disclosure provides a pharmaceutical composition comprising a compound of Formula (I), (II), (III), or (IV), or a pharmaceutically acceptable salt thereof, and one or more pharmaceutically acceptable carriers or excipients.

As used herein, the term “composition” is intended to encompass a product comprising the specified ingredients in the specified amounts, as well as any product which results, directly or indirectly, from combination of the specified ingredients in the specified amounts.

The compounds of Formula (I), (II), (III), or (IV) can be formulated for oral administration in forms such as tablets, capsules (each of which includes sustained release or timed release formulations), pills, powders, granules, elixirs, tinctures, suspensions, syrups, and emulsions. The compounds of Formula (I), (II), (III), or (IV) can also be formulated for intravenous (bolus or in-fusion), intraperitoneal, topical, subcutaneous, intramuscular, or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

According to a further aspect of the disclosure there is provided a pharmaceutical composition which comprises a compound any one of the Formulae disclosed herein, or a pharmaceutically acceptable salt thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

### Methods of Use

In some aspects, the present disclosure provides a method of modulating PI3K (e.g., PI3Kα) activity (e.g., in vitro or in vivo), comprising contacting a cell with a therapeutically effective amount of a compound of Formula (I), (II), (III), or (IV), or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of Formula (I), (II), (III), or (IV), or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of Formula (I), (II), (III), or (IV), or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of Formula (I), (II), (III), or (IV), or a pharmaceutically acceptable salt thereof, in combination with an effective amount of one or more therapeutic agents.

In some aspects, the present disclosure provides a method of treating a disease or disorder disclosed herein in a subject in need thereof, comprising administering to the subject a therapeutically effective amount of a compound of Formula (I), (II), (III), or (IV), or a pharmaceutically acceptable salt thereof, in combination with an effective amount of: a CDK4 and 6 inhibitor, or a pharmaceutically acceptable salt thereof; a SERD, or a pharmaceutically acceptable salt thereof; an aromatase inhibitor, or a pharmaceutically acceptable salt thereof, a taxane, or a pharmaceutically acceptable salt thereof; an mTOR inhibitor, or a pharmaceutically acceptable salt thereof; a tyrosine kinase inhibitor, or a pharmaceutically acceptable salt thereof; a platinum agent; an anthracycline, or a pharmaceutically acceptable salt thereof, an immune checkpoint inhibitor, or a pharmaceutically acceptable salt thereof; an antiandrogen, or a pharmaceutically acceptable salt thereof, an anti-HER2 monoclonal antibody; an anti-HER2 antibody-drug conjugate; a KRAS inhibitor, or a pharmaceutically acceptable salt thereof: an MEK inhibitor, or a pharmaceutically acceptable salt thereof; an ERK inhibitor, or a pharmaceutically acceptable salt thereof; a topoisomerase inhibitor, or a pharmaceutically acceptable salt thereof; a SERM, or a pharmaceutically acceptable salt thereof; or a PARP inhibitor, or a pharmaceutically acceptable salt thereof; or a combination thereof.

In some embodiments, the disease or disorder is associated with an implicated PI3K activity.

In some embodiments, the disease or disorder is a disease or disorder in which PI3K activity is implicated.

In some embodiments, the disease or disorder is a cancer.

In some embodiments, the cancer is selected from acute lymphoblastic leukemia (ALL), acute myeloid leukemia (AML), adrenocortical carcinoma, aids-related cancers, aids-related lymphoma, anal cancer, astrocytoma, basal cell carcinoma, bile duct cancer, bladder cancer, bone cancer, osteosarcoma, malignant fibrous histiocytoma, brain tumors, breast cancer, bronchial tumors, Burkitt lymphoma, carcinoid tumor, cancer of unknown primary, cardiac (heart) tumors, atypical teratoid/rhabdoid tumor, primary CNS lymphoma, cervical cancer, cholangiocarcinoma, chordoma, chronic lymphocytic leukemia (CLL), chronic myelogenous leukemia (CML), colorectal cancer, craniopharyngioma, cutaneous t-cell lymphoma, mycosis fungoides, Sezary syndrome, ductal carcinoma in situ (DCIS), embryonal tumors, medulloblastoma, endometrial cancer, ependymoma, esophageal cancer, esthesioneuroblastoma, Ewing sarcoma, extracranial germ cell tumor, extragonadal germ cell tumor, fallopian tube cancer, gallbladder cancer, gastric cancer, gastrointestinal carcinoid tumor, malignant gastrointestinal stromal tumors (GIST), germ cell tumors, gestational trophoblastic disease, hairy cell leukemia, head and neck cancer, hepatocellular cancer, Langerhans cell histiocytosis, Hodgkin lymphoma, islet cell tumors, pancreatic neuroendocrine tumors, Kaposi sarcoma, kidney cancer, laryngeal cancer, leukemia, liver cancer, lung cancer, lymphoma, male breast cancer, intraocular melanoma, Merkel cell carcinoma, malignant mesothelioma, metastatic cancer, metastatic squamous neck cancer, midline tract carcinoma with nut gene changes, mouth cancer, multiple endocrine neoplasia syndromes, multiple myeloma/plasma cell neoplasms, myelodysplastic syndromes, myelodysplastic neoplasms, myeloproliferative neoplasms, chronic myeloproliferative neoplasm, nasal cavity and paranasal sinus cancer, nasopharyngeal cancer, neuroblastoma, non-Hodgkin lymphoma, non-small cell lung cancer, oral cancer, lip and oral cavity cancer, oropharyngeal cancer, malignant fibrous histiocytoma of bone, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, multiple myeloma, pleuropulmonary blastoma, primary central nervous system (CNS) lymphoma, primary peritoneal cancer, prostate cancer, rectal cancer, recurrent cancer, renal cell (kidney) cancer, retinoblastoma, rhabdomyosarcoma, salivary gland cancer, sarcoma, childhood vascular tumors, skin cancer, small cell lung cancer, small intestine cancer, soft tissue sarcoma, squamous cell carcinoma of the skin, testicular cancer, oropharyngeal cancer, hypopharyngeal cancer, thymoma, thymic carcinoma, thyroid cancer, tracheobronchial tumors, transitional cell cancer of the renal pelvis and ureter, urethral cancer, uterine sarcoma, vaginal cancer, vascular tumors, vulvar cancer, and Wilms tumor.

In some embodiments, the cancer is Endometrial cancer, Breast cancer, Oesophageal squamous-cell cancer, Cervical squamous-cell carcinoma, Cervical adenocarcinoma, Colorectal adenocarcinoma, Bladder Urothelial Carcinoma, Glioblastoma, Ovarian cancer, Non-small-cell Lung cancer, Esophagogastric cancer, Nerve-sheath tumor, Head and neck squamous-cell carcinoma, Melanoma, Esophagogastric adenocarcinoma, Soft-tissue sarcoma, Prostate cancer, Fibrolamellar carcinoma, Hepatocellular carcinoma, Diffuse glioma, Colorectal cancer, Pancreatic cancer, Cholangiocarcinoma, B-cell lymphoma, Mesothelioma, Adrenocortical carcinoma, Renal non-clear-cell carcinoma, Renal clear-cell carcinoma, Germ-cell carcinoma, Thymic tumor, Pheochromocytoma, Miscellaneous neuroepithelial tumor, thyroid cancer, leukemia, or encapsulated glioma.

In some embodiments, the cancer is a breast cancer, a prostate cancer, or a brain cancer.

In some embodiments, the cancer is a breast cancer. In some embodiments, the cancer is a prostate cancer. In some embodiments, the cancer is a brain cancer.

In some embodiments, the breast cancer is metastatic breast cancer. In some embodiments, the breast cancer is ductal carcinoma in situ (DCIS). In some embodiments, the breast cancer is invasive ductal carcinoma. In some embodiments, the breast cancer is triple negative breast cancer. In some embodiments, the breast cancer is medullary carcinoma. In some embodiments, the breast cancer is tubular carcinoma. In some embodiments, the breast cancer is mucinous carcinoma. In some embodiments, the breast cancer is Paget disease of the breast or nipple. In some embodiments, the breast cancer is inflammatory breast cancer (IBC). In some embodiments, the breast cancer is hormone receptor-positive (HR+), human epidermal growth factor receptor 2-negative (HER2−) advanced or metastatic breast cancer.

In some embodiments, the prostate cancer is an adenocarcinoma. In some embodiments, the prostate cancer is a small cell carcinoma. In some embodiments, the prostate cancer is a neuroendocrine tumor. In some embodiments, the prostate cancer is a transitional cell carcinoma. In some embodiments, the prostate cancer is a sarcoma.

In some embodiments, the brain cancer is an acoustic neuroma. In some embodiments, the brain cancer is an astrocytoma. In some embodiments, the brain cancer is a brain metastasis. In some embodiments, the brain cancer is choroid plexus carcinoma. In some embodiments, the brain cancer is craniopharyngioma. In some embodiments, the brain cancer is an embryonal tumor. In some embodiments, the brain cancer is an ependymoma. In some embodiments, the brain cancer is a glioblastoma. In some embodiments, the brain cancer is a glioma. In some embodiments, the brain cancer is a medulloblastoma. In some embodiments, the brain cancer is a meningioma. In some embodiments, the brain cancer is an oligodendroglioma. In some embodiments, the brain cancer is a pediatric brain tumor. In some embodiments, the brain cancer is a pineoblastoma. In some embodiments, the brain cancer is a pituitary tumor.

In some embodiments, the disease or disorder associated with PI3K includes, but is not limited to, CLOVES syndrome, PROS, breast cancer, brain cancer, prostate cancer, endometrial cancer, gastric cancer, leukemia, lymphoma, sarcoma, colorectal cancer, lung cancer, ovarian cancer, skin cancer, or head and neck cancer.

In some embodiments, the diseases or disorder associated with PI3K is CLOVES syndrome.

In some embodiments, the disease or disorder associated with PI3K is PROS.

In some embodiments, the disease or disorder associated with PI3K is breast cancer, brain cancer, prostate cancer, endometrial cancer, gastric cancer, leukemia, lymphoma, sarcoma, colorectal cancer, lung cancer, ovarian cancer, skin cancer, or head and neck cancer.

In some embodiments, the disease or disorder associated with PI3K is a breast neoplasm, a thyroid neoplasm, an ovarian neoplasm, non-small-cell lung carcinoma, an endometrial neoplasm, or a pancreatic neoplasm. In some embodiments, the disease or disorder associated with PI3K is a breast neoplasm. In some embodiments, the disease or disorder associated with PI3K is a thyroid neoplasm. In some embodiments, the disease or disorder associated with PI3K is an ovarian neoplasm. In some embodiments, the disease or disorder associated with PI3K is non-small-cell lung carcinoma. In some embodiments, the disease or disorder associated with PI3K is an endometrial neoplasm. In some embodiments, the disease or disorder associated with PI3K is a pancreatic neoplasm.

In some embodiments, the disease or disorder associated with PI3K is breast cancer, brain cancer, prostate cancer, endometrial cancer, gastric cancer, colorectal cancer, lung cancer, ovarian cancer, skin cancer, or head and neck cancer.

In some embodiments, the disease or disorder associated with PI3K is leukemia, lymphoma, or sarcoma.

In some embodiments, the cancer is endometrial cancer, head and neck cancer, or a sarcoma.

In some embodiments, the cancer is endometrial cancer. In some embodiments the cancer is head and neck cancer. In some embodiments, the cancer is a sarcoma.

In some embodiments, the sarcoma is soft tissue sarcoma, osteosarcoma, chondrosarcoma, Ewing sarcoma, hemangioendothelioma, angiosarcoma, fibrosarcoma, myofibrosarcoma, chordoma, adamantinoma, liposarcoma, leiomyosarcoma, malignant peripheral nerve sheath tumor, rhabdomyosarcoma, synovial sarcoma, or malignant solitary fibrous tumor.

In some embodiments, the sarcoma is soft tissue sarcoma. In some embodiments the soft tissue sarcoma is liposarcoma, atypical lipomatous tumor, dermatofibrosarcoma protuberans, malignant solitary fibrous tumor, inflammatory myofibroblastic tumor, low-grade myofibroblastic sarcoma, fibrosarcoma, myxofibrosarcoma, low-grade fibromyxoid sarcoma, giant cell tumor of soft tissues, leiomyosarcoma, malignant glomus tumor, rhabdomyosarcoma, hemangioendothelioma, angiosarcoma of soft tissue, extraskeletal osteosarcoma, gastrointestinal stromal tumor, malignant gastrointestinal stromal tumor (GIST), malignant peripheral nerve sheath tumor, malignant Triton tumor, malignant granular cell tumor, malignant ossifying fibromyxoid tumor, stromal sarcoma, myoepithelial carcinoma, malignant phosphaturic mesenchymal tumor, synovial sarcoma, epithelioid sarcoma, alveolar soft part sarcoma, clear cell sarcoma of soft tissue, extraskeletal myxoid chondrosarcoma, extraskeletal Ewing sarcoma, desmoplastic small round cell tumor, extrarenal rhabdoid tumor, perivascular epithelioid cell tumor, intimal sarcoma, undifferentiated spindle cell sarcoma, undifferentiated pleomorphic sarcoma, undifferentiated round cell sarcoma, undifferentiated epithelioid sarcoma, or undifferentiated sarcoma, not otherwise specified.

In some aspects, the present disclosure provides a method of treating or preventing a cancer in a subject in need thereof, comprising administering to the subject a therapeutically effective amount of a compound of Formula (I), (II), (III), or (IV), or a pharmaceutically acceptable salt thereof, or a pharmaceutical composition of the present disclosure.

In some aspects, the present disclosure provides a method of treating a cancer in a subject in need thereof, comprising administering to the subject a therapeutically effective amount of a compound of Formula (I), (II), (III), or (IV), or a pharmaceutically acceptable salt thereof, or a pharmaceutical composition of the present disclosure.

In some aspects, the present disclosure provides a method of treating or preventing a breast cancer in a subject in need thereof, comprising administering to the subject a therapeutically effective amount of a compound of Formula (I), (II), (III), or (IV), or a pharmaceutically acceptable salt thereof, or a pharmaceutical composition of the present disclosure.

In some aspects, the present disclosure provides a method of treating a breast cancer in a subject in need thereof, comprising administering to the subject a therapeutically effective amount of a compound of Formula (I), (II), (III), or (IV), or a pharmaceutically acceptable salt thereof, or a pharmaceutical composition of the present disclosure.

In some aspects, the present disclosure provides a method of treating or preventing a prostate cancer in a subject in need thereof, comprising administering to the subject a therapeutically effective amount of a compound of Formula (I), (II), (III), or (IV), or a pharmaceutically acceptable salt thereof, or a pharmaceutical composition of the present disclosure.

In some aspects, the present disclosure provides a method of treating a prostate cancer in a subject in need thereof, comprising administering to the subject a therapeutically effective amount of a compound of Formula (I), (II), (III), or (IV), or a pharmaceutically acceptable salt thereof, or a pharmaceutical composition of the present disclosure.

In some aspects, the present disclosure provides a method of treating or preventing a brain cancer in a subject in need thereof, comprising administering to the subject a therapeutically effective amount of a compound of Formula (I), (II), (III), or (IV), or a pharmaceutically acceptable salt thereof, or a pharmaceutical composition of the present disclosure.

In some aspects, the present disclosure provides a method of treating a brain cancer in a subject in need thereof, comprising administering to the subject a therapeutically effective amount of a compound of Formula (I), (II), (III), or (IV), or a pharmaceutically acceptable salt thereof, or a pharmaceutical composition of the present disclosure.

In some aspects, the present disclosure provides a compound of Formula (I), (II), (III), or (IV), or a pharmaceutically acceptable salt thereof for use in therapy.

In some aspects, the present disclosure provides a compound of Formula (I), (II), (III), or (IV), or a pharmaceutically acceptable salt thereof for use in modulating PI3K (e.g., PI3Kα) activity (e.g., in vitro or in vivo).

In some aspects, the present disclosure provides a compound of Formula (I), (II), (III), or (IV), or a pharmaceutically acceptable salt thereof for use in treating or preventing a disease or disorder disclosed herein.

In some aspects, the present disclosure provides a compound of Formula (I), (II), (III), or (IV), or a pharmaceutically acceptable salt thereof for use in treating a disease or disorder disclosed herein.

In some aspects, the present disclosure provides a compound of Formula (I), (II), (III), or (IV), or a pharmaceutically acceptable salt thereof for use in treating or preventing a cancer.

In some aspects, the present disclosure provides a compound of Formula (I), (II), (III), or (IV), or a pharmaceutically acceptable salt thereof for use in treating a cancer.

In some aspects, the present disclosure provides a compound of Formula (I), (II), (III), or (IV), or a pharmaceutically acceptable salt thereof for use in treating or preventing a breast cancer.

In some aspects, the present disclosure provides a compound of Formula (I), (II), (III), or (IV), or a pharmaceutically acceptable salt thereof for use in treating a breast cancer.

In some aspects, the present disclosure provides a compound of Formula (I), (II), (III), or (IV), or a pharmaceutically acceptable salt thereof for use in treating or preventing a prostate cancer.

In some aspects, the present disclosure provides a compound of Formula (I), (II), (III), or (IV), or a pharmaceutically acceptable salt thereof for use in treating a prostate cancer.

In some aspects, the present disclosure provides a compound of Formula (I), (II), (III), or (IV), or a pharmaceutically acceptable salt thereof for use in treating or preventing a brain cancer.

In some aspects, the present disclosure provides a compound of Formula (I), (II), (III), or (IV), or a pharmaceutically acceptable salt thereof for use in treating a brain cancer.

In some aspects, the present disclosure provides use of a compound of Formula (I), (II), (III), or (IV), or a pharmaceutically acceptable salt thereof in the manufacture of a medicament for modulating PI3K (e.g., PI3Kα) activity (e.g., in vitro or in vivo).

In some aspects, the present disclosure provides use of a compound of Formula (I), (II), (III), or (IV), or a pharmaceutically acceptable salt thereof in the manufacture of a medicament for treating or preventing a disease or disorder disclosed herein.

In some aspects, the present disclosure provides use of a compound of Formula (I), (II), (III), or (IV), or a pharmaceutically acceptable salt thereof in the manufacture of a medicament for treating a disease or disorder disclosed herein.

In some aspects, the present disclosure provides use of a compound of Formula (I), (II), (III), or (IV), or a pharmaceutically acceptable salt thereof in the manufacture of a medicament for treating or preventing a cancer in a subject in need thereof.

In some aspects, the present disclosure provides use of a compound of Formula (I), (II), (III), or (IV), or a pharmaceutically acceptable salt thereof in the manufacture of a medicament for treating a cancer in a subject in need thereof.

In some aspects, the present disclosure provides use of a compound of Formula (I), (II), (III), or (IV), or a pharmaceutically acceptable salt thereof in the manufacture of a medicament for treating or preventing a breast cancer in a subject in need thereof.

In some aspects, the present disclosure provides use of a compound of Formula (I), (II), (III), or (IV), or a pharmaceutically acceptable salt thereof in the manufacture of a medicament for treating a breast cancer in a subject in need thereof.

In some aspects, the present disclosure provides use of a compound of Formula (I), (II), (III), or (IV), or a pharmaceutically acceptable salt thereof in the manufacture of a medicament for treating or preventing a prostate cancer in a subject in need thereof.

In some aspects, the present disclosure provides use of a compound of Formula (I), (II), (III), or (IV), or a pharmaceutically acceptable salt thereof in the manufacture of a medicament for treating a prostate cancer in a subject in need thereof.

In some aspects, the present disclosure provides use of a compound of Formula (I), (II), (III), or (IV), or a pharmaceutically acceptable salt thereof in the manufacture of a medicament for treating or preventing a brain cancer in a subject in need thereof.

In some aspects, the present disclosure provides use of a compound of Formula (I), (II), (III), or (IV), or a pharmaceutically acceptable salt thereof in the manufacture of a medicament for treating a brain cancer in a subject in need thereof.

The present disclosure provides compounds that function as modulators of PI3K activity. The present disclosure therefore provides a method of modulating PI3K activity in vitro or in vivo, said method comprising contacting a cell with a therapeutically effective amount of a compound, or a pharmaceutically acceptable salt thereof, as defined herein.

In some embodiments, PI3K modulation is inhibition of PI3K.

In some embodiments, the PI3K inhibitor is a PI3Kα inhibitor. In some embodiments, the PI3K inhibitor is a PI3Kα H1047R mutant inhibitor. In some embodiments, the PI3K inhibitor is a PI3Kα E575K mutant inhibitor. In some embodiments, the PI3K inhibitor is both of a PI3Kα H1047R mutant inhibitor and a PI3Kα E575K mutant inhibitor.

Effectiveness of compounds of the disclosure can be determined by industry-accepted assays/disease models according to standard practices of elucidating the same as described in the art and are found in the current general knowledge.

The present disclosure also provides a method of treating a disease or disorder in which PI3K activity is implicated in a patient in need of such treatment, said method comprising administering to said patient a therapeutically effective amount of a compound, or a pharmaceutically acceptable salt thereof, or a pharmaceutical composition as defined herein.

### Routes of Administration

The compounds of Formula (I), (II), (III), or (IV), or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### EXAMPLES

Exemplary compounds of Formula (I), (II), (III), and (IV) are synthesized and tested in the examples. It is understood that compounds of Formula (I), (II), (III), and (IV) may be converted to the corresponding pharmaceutically acceptable salts of the compounds using routine techniques in the art.

Nuclear magnetic resonance (NMR) spectra were recorded at 400 MHz or 300 MHz as stated and at 300.3 K unless otherwise stated; the chemical shifts (6) are reported in parts per million (ppm). Spectra were recorded using a Bruker or Varian instrument with 8, 16 or 32 scans.

LC-MS chromatograms and spectra were recorded using an Agilent 1200 or Shimadzu LC-20 AD&MS 2020 instrument using a C-18 column such as a Luna-C18 2.0×30 mm or Xbridge Shield RPC18 2.1×50 mm. Injection volumes were 0.7-8.0 μl and the flow rates were typically 0.8 or 1.2 ml/min. Detection methods were diode array (DAD) or evaporative light scattering (ELSD) as well as positive ion electrospray ionization. MS range was 100-1000 Da. Solvents were gradients of water and acetonitrile both containing a modifier (typically 0.01-0.04%) such as trifluoroacetic acid or ammonium carbonate.

### Abbreviations

ACN Acetonitrile
AcOH Acetic Acid
ADP Adenosine diphosphate
ATP Adenosine triphosphate
CDCl₃ Chloroform-d
DCM Dichloromethane
DIAD Diisopropyl azodicarboxylate
DMF N,N-dimethylformamide
DMSO Dimethylsulfoxide
DMSO-d₆ Hexadeuterodimethylsulfoxide
eq equivalents
EtOAc Ethyl Acetate
EtOH Ethanol
h hour(s)
¹H NMR Proton nuclear magnetic resonance spectroscopy
IPA Isopropanol
K₂CO₃ Potassium carbonate
LC-MS Liquid Chromatography-Mass Spectrometry
MeOH Methanol
MgSO₄ Magnesium sulfate
min minute(s)
MS ES Mass Spectroscopy Electro Spray
MTBE Methyl tert-butyl ether
NH₄HCO₃ Ammonium bicarbonate
ppm parts per million
rt room temperature
SFC Supercritical Fluid Chromatography
TIHF Tetrahydrofuran

### Intermediate 1: (2-Bromo-4-methyl-phenyl) propanoate

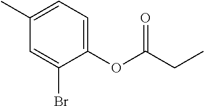

A mixture of 2-bromo-4-methyl-phenol (10.0 g, 53.5 mmol) and pyridine (6.34 g, 80.2 mmol) in DCM (100 mL) was treated with propanoyl chloride (5.44 g, 58.8 mmol) at 0° C. and stirred at 25° C. for 16 h. The mixture was diluted with water (100 mL), the pH adjusted to 5 with HCl (2 M), and extracted with DCM (2×10⁰ mL). The combined organic extracts were washed with saturated aqueous sodium chloride (2×150 mL), dried over anhydrous Na₂SO₄, filtered, and concentrated to give the product as an oil (13 g, crude). ¹H NMR (400 MHz, DMSO-d6) δ ppm 1.17 (t, J=7.6 Hz, 3H), 2.30 (s, 3H), 2.62 (q, J=7.6 Hz, 2H), 7.11-7.18 (m, 1H), 7.19-7.26 (m, 1H), 7.50-7.55 (m, 1H).

### Intermediate 1B: (2-Bromo-4-methyl-phenyl) acetate

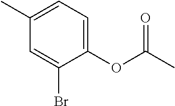

A DCM (2.4 L) mixture of 2-bromo-4-methyl-phenol (300 g, 1.6 mol) and pyridine (152 g, 1.92 mol) at 0° C. was treated with acetyl chloride and stirred at 25° C. for 16 h. The mixture was diluted with water (1500 mL), the pH adjusted to 5 with HCl (2 M aqueous), and extracted with DCM (3×500 mL). The combined organic extracts were washed with saturated aqueous sodium chloride (2×250 mL), dried over Na₂SO₄, filtered, and concentrated to give the product as an oil (400 g, crude). ¹H NMR (400 MHz, CDCl₃) δ ppm 2.24 (s, 3H), 2.25 (s, 3H), 6.91 (d, J=8.4 Hz, 2H), 7.01-7.02 (m, 2H), 7.33 (s, 1H).

### Intermediate 2: 1-(3-Bromo-2-hydroxy-5-methyl-phenyl)propan-1-one

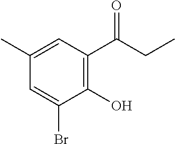 [0425] Route 1: A mixture of (2-bromo-4-methyl-phenyl) propanoate (12.5 g, 51.4 mmol) and AlCl₃ (24.0 g, 180 mmol) was stirred at 140° C. for 1 h. When cooled to rt, the mixture was quenched with water (80 mL) dropwise and stirred for 30 min. The mixture was extracted with EtOAc (3×10⁰ mL). The combined organic extracts were washed with saturated aqueous sodium chloride (2×200 mL), dried over anhydrous Na₂SO₄, and filtered. The filtrate was concentrated and triturated with petroleum ether (20 mL) to give the product as a solid (9.82 g, 79%). ¹H NMR (400 MHz, DMSO-d6) δ ppm 1.10 (t, J=7.2 Hz, 3H), 2.28 (s, 3H), 3.15 (q, J=7.2 Hz, 2H), 7.66-7.73 (m, 1H), 7.77-7.83 (m, 1H), 12.66 (s, 1H).
Route 2: (2-Bromo-4-methyl-phenyl) propanoate (120 g, 496 mmol) was transferred to a reactor, cooled to −20° C., and treated with trifluoromethanesulfonic acid (216 mL). After addition was complete, the reaction was stirred at 60° C. for 1 h. The reaction was cooled to rt and poured into icy water (600 mL). The product was removed by filtration (114 g, 95%) as a yellow solid. MS ES-m/z 241, 243 [M−H]⁻. ¹H NMR (400 MHz, CDCl₃) δ ppm 1.26 (t, 3H), 2.33 (s, 3H), 3.06 (q, 2H), 7.55 (m, 2H), 12.87 (s, 1H).

### Intermediate 2B: 1-(3-Bromo-2-hydroxy-5-methyl-phenyl)ethanone

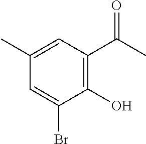

A mixture of (2-bromo-4-methyl-phenyl) acetate (50 g, 218 mmol) and AlCl₃ (102 g, 764 mmol) was degassed and purged with N2 three times and stirred at 140° C. for 1 h. After cooling to rt, the reaction was diluted with DCM (30 mL) and dropped into 150 mL of water at 0° C. The mixture was filtered and the aqueous phase extracted with DCM (2×150 mL). The combined organic extracts were washed with saturated aqueous sodium chloride, dried over anhydrous Na₂SO₄, filtered, and concentrated. The residue was triturated with petroleum ether (2×150 mL) to give the product as a solid (30 g, 52%). ¹H NMR (400 MHz, CDCl₃) δ ppm 2.30 (s, 3H), 2.68 (s, 3H), 7.73 (s, 1H), 7.33 (s, 1H), 12.64 (s, 1H).

### Intermediate 3B: 1-(3-Bromo-4-chloro-2-hydroxy-5-methyl-phenyl)propan-1-one

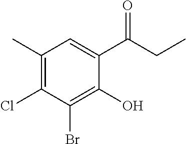

A mixture of 1-(4-chloro-2-hydroxy-5-methyl-phenyl)propan-1-one (6.5 g, 33 mmol) and sodium acetate (4.0 g, 49 mmol) in acetic acid (50 mL) was treated with the dropwise addition of bromine (1.7 mL, 33 mmol). The reaction mixture was stirred at rt for 12 h, then diluted with water (100 mL) and EtOAc (200 mL). The organic phase was concentrated under reduced pressure and the residue was purified by silica gel chromatography eluted with 0% to 100% EtOAc in heptane to give the title compound (7.8 g, 86%). MS ES+ m/z 277, 279 [M+H]⁺.

### Intermediate 3: (E)-1-(3-Bromo-2-hydroxy-5-methyl-phenyl)-2-methyl-3-phenyl-prop-2-en-1-one

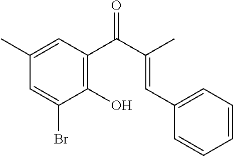 [SMILES: C/C(=C\c1ccccc1)C(=O)c1cc(C)cc(Br)c1O]

A mixture of 1-(3-bromo-2-hydroxy-5-methyl-phenyl)propan-1-one (200 g, 822.72 mmol), benzaldehyde (96.04 g, 904.99 mmol), AcOH (105.23 g, 1.75 mol), and piperidine (172.33 g, 2.02 mol) in EtOH (1600 mL) was stirred at 70° C. for 16 h. The resulting dark solution was poured into water (3 L), filtered, and the solid dissolved in 6 L of DCM. The organic solution was dried over anhydrous Na₂SO₄, filtered, and concentrated to give the product as a dark gum. MS ES+ m/z 331, 333 [M+H]⁺.

### Intermediate 4B: (E)-1-(3-Bromo-4-chloro-2-hydroxy-5-methyl-phenyl)-2-methyl-3-(3-pyridyl)prop-2-en-1-one

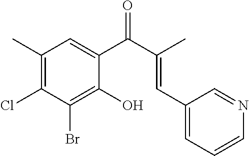 [SMILES: C/C(=C\c1cccnc1)C(=O)c1cc(C)c(Cl)c(Br)c1O]

A mixture of 1-(3-bromo-4-chloro-2-hydroxy-5-methyl-phenyl)propan-1-one (1.00 g, 3.60 mmol), nicotinaldehyde (0.463 g, 4.32 mmol) and potassium hydroxide (0.809 g, 14.4 mmol) in EtOH (10 mL) was stirred at rt for 2 h. The mixture was diluted with water (100 mL) and extracted with EtOAc (100 mL). The organic layer was concentrated under reduced pressure to give the crude title compound (1.3 g, 98%). MS ES+ m/z 366, 368 [M+H]⁺.

### Intermediate 1A: 8-Bromo-4-hydroxy-3,6-dimethyl-chromene-2-thione

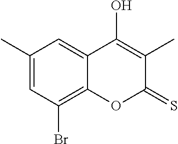

A solution of 1-(3-bromo-2-hydroxy-5-methyl-phenyl)propan-1-one (495 g, 2.04 mol) and carbon disulfide (233 g, 3.06 mol) in THE (4.95 L) under nitrogen was cooled to about −25° C. and treated dropwise with sodium bis(trimethylsilyl)amide (2M in THF, 3.57 L, 1.79 mol). When addition was complete, the reaction was allowed to warm to rt over 16 h. The reaction was added dropwise to 15% aqueous H₂SO₄ (4.95 L) at 0 to 10° C. and the pH of the mixture maintained at 2 to 3. The phases were separated and the aqueous layer extracted with MTBE (495 mL). The organic layers were combined, concentrated under reduced pressure, and the residue suspended in heptane (1.49 L). The suspension was stirred for 5 h at rt and the title compound (557.9 g, 96%) was collected by filtration as a yellow solid. ¹H NMR (400 MHz, DMSO-d6) δ ppm 2.24 (s, 3H), 2.38 (s, 3H), 7.76 (s, 1H), 7.79 (s, 1H).

### Intermediate 5B: 8-Bromo-4-hydroxy-6-methyl-chromene-2-thione

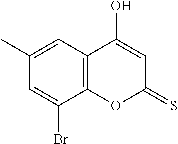

A solution of 1-(3-bromo-2-hydroxy-5-methyl-phenyl)ethanone (65 g, 284 mmol) in THE (800 mL) was treated with NaHMDS (851 mL, 1 M) at −50° C. over 30 min, allowed to warm to between −5° C. and 0° C., and stirred for 1 h. The reaction was cooled to −20° C. and treated with CS₂ (64.8 g, 851 mmol) dropwise over 1 h, allowed to warm to 25° C., and stirred for another 16 h. The reaction was quenched with H₂SO₄ (800 mL, 15%) at −50° C. over 1 h, allowed to warm to rt, and extracted with EtOAc (2×1 L). The combined organic extracts were washed with saturated aqueous sodium chloride (1 L), dried over anhydrous Na₂SO₄, filtered, and concentrated. The residue was triturated with EtOAc (0.5 L) to give the product as a solid (210 g crude, 64%, purity ˜76%).

### Intermediate 2A: 8-Bromo-2-ethylsulfanyl-3,6-dimethyl-chromen-4-one

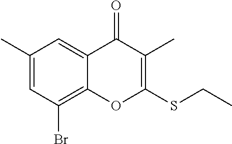 [SMILES: CCSc1oc2c(Br)cc(C)cc2c(=O)c1C]

A mixture of 8-bromo-4-hydroxy-3,6-dimethyl-chromene-2-thione (560 g, 1.96 mol), K₂CO₃ (271.4 g, 1.96 mol), and ethyl iodide (459.5 g, 2.95 mol) in acetone (5.60 L) was stirred at 20 to 25° C. for 2 h. The reaction was filtered and the solids washed with THE (1.12 L). The filtrate was concentrated under reduced pressure and the residue purified by silica gel chromatography eluted with 1% to 33% EtOAc in heptane to give the title compound (545.5 g, 88%) as a brown solid. ¹H NMR (400 MHz, CDCl₃) δ ppm 1.50 (t, J=7.36 Hz, 3H), 2.05 (s, 3H), 2.43 (s, 3H), 3.31 (q, J=7.36 Hz, 2H), 7.64 (s, 1H), 7.93 (s, 1H).

### Intermediate 6B: 8-Bromo-2-ethylsulfanyl-6-methyl-chromen-4-one

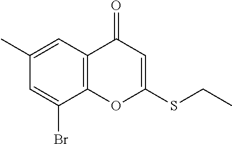 [SMILES: CCSc1cc(=O)c2cc(C)cc(Br)c2o1]

A mixture of 8-bromo-4-hydroxy-6-methyl-chromene-2-thione (20.0 g, 73.8 mmol), EtI (46 g, 295 mmol), and K₂CO₃ (12.2 g, 88.5 mmol) in acetone (200 mL) was stirred at 60° C. for 3 h. When the reaction had cooled to rt, the mixture was diluted with water (200 mL) and extracted with DCM (2×200 mL). The combined organic extracts were concentrated and purified by silica gel chromatography eluted with 20% to 40% EtOAc in petroleum ether to give the product as a gum. ¹H NMR (400 MHz, CDCl₃) δ ppm 1.51 (t, J=7.2 Hz, 3H), 2.45 (s, 3H), 3.22 (q, J=7.2 Hz, 2H), 6.32 (s, 1H), 7.70 (s, 1H), 7.93 (s, 1H).

### Intermediate 4: 8-Bromo-3,6-dimethyl-2-phenyl-chromen-4-one

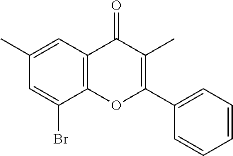 [SMILES: Cc1cc(Br)c2oc(-c3ccccc3)c(C)c(=O)c2c1] [0444] Route 1: A mixture of (E)-1-(3-bromo-2-hydroxy-5-methyl-phenyl)-2-methyl-3-phenyl-prop-2-en-1-one (284 g, 857.48 mmol) and iodine (21.76 g, 85.75 mmol, 17.27 mL, 0.1 eq) in DMSO (1200 mL) was stirred at 140° C. for 2 h to give a black-brown solution. Cooled to rt, poured the reaction into 3 L of water, filtered, dissolved the solid product in DCM (4 L), dried over anhydrous Na₂SO₄, filtered, and concentrated to give a residue. The residue was triturated with petroleum ether/EtOAc (1:1, 1 L) to give the product as a light yellow solid (195 g, 69%). MS ES+ m/z 329, 331 [M+H]⁺.
Route 2: A solution of 1-(3-bromo-2-hydroxy-5-methyl-phenyl)propan-1-one (50.0 g, 205.7 mmol) in THE (100 mL) was cooled to −80° C. and treated with lithium bis(trimethylsilyl)amide (1M in THF, 617 mmol). After stirring at −80° C. for 1 h, the mixture was warmed to 0° C. and stirred for 1 h. The mixture was cooled to −80° C. and treated dropwise with benzoyl chloride (37.6 g, 267.4 mmol). After addition was complete, the reaction was allowed to stir at 25° C. for 16 h. The reaction was cooled to −20° C. and the pH adjusted to 4 with 50% aqueous acetic acid. The THF was removed under vacuum and the precipitate removed by filtration. The solid was dissolved in acetic acid and aqueous HCl (250 mL/10 mL) and the resulting solution stirred at 100° C. for 1 h. The mixture was cooled to 20° C. and diluted with water (100 mL). A solid was removed by filtration and washed with water (50 mL) and triturated with 200 mL of EtOAc at rt for 30 min. The product was collected by filtration (170.7 g, 83%) as an off-white solid. MS ES+ m/z 329, 331 [M+H]⁺. ¹H NMR (400 MHz, CDCl₃) δ ppm 2.24 (s, 3H), 2.48 (s, 3H), 7.55-7.57 (m, 3H), 7.75-7.78 (m, 3H), 8.00 (s, 1H).

The following compound in Table 1 was made in a similar way as described for 8-bromo-3,6-dimethyl-2-phenyl-chromen-4-one, Route 2.

TABLE 1

| | | | MS ES+ |
|---|---|---|---|
| Interm # | Chemical Name | Structure | m/z |
| 5 | 8-Bromo-3,6-dimethyl-2- (3-pyridyl)chromen-4- one | 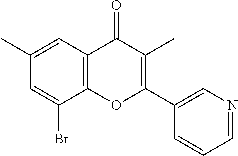 [SMILES: Cc1cc(Br)c2oc(-c3cccnc3)c(C)c(=O)c2c1] | 330, 332 [M + H]⁺ |

### Intermediate 7B: 8-Bromo-7-chloro-3,6-dimethyl-2-(3-pyridyl)chromen-4-one

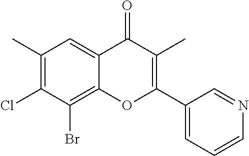 [SMILES: Cc1cc2c(=O)c(C)c(-c3cccnc3)oc2c(Br)c1Cl]

A mixture of (E)-1-(3-bromo-4-chloro-2-hydroxy-5-methyl-phenyl)-2-methyl-3-(3-pyridyl)prop-2-en-1-one (1.2 g, 3.3 mmol) and iodine (0.083 g, 0.33 mmol) in DMSO (5 mL) was heated at 90° C. for 3 h. The reaction mixture was loaded onto a C18 column and purified by reversed phase chromatography eluted with 60% ACN in water (with trifluoroacetic acid additive) to give the title compound (0.950 g, 80%). MS ES+ m/z 364, 366 [M+H]⁺.

### Intermediate 6: 8-Acetyl-3,6-dimethyl-2-phenyl-chromen-4-one

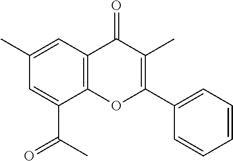 [SMILES: CC(=O)c1cc(C)cc2c(=O)c(C)c(-c3ccccc3)oc12] [0450] Route 1: A mixture of 8-bromo-3,6-dimethyl-2-phenyl-chromen-4-one (195 g, 592.37 mmol), bis(triphenylphosphine)palladium(II) dichloride (20.79 g, 29.62 mmol), and tributyl(1-ethoxyvinyl)stannane (256.72 g, 710.84 mmol, 239.92 mL) in dioxane (1600 mL) was stirred under N2 at 95° C. for 16 h to give a black-brown solution. After cooling to rt, treated the reaction with 1M aqueous HCl (100 mL) and stirred at 20° C. for 30 min. The mixture was quenched with saturated aqueous KF (2000 mL), stirred for 30 min, and filtered. The filter cake was washed with 10% MeOH in DCM (5×5000 mL). The combined extracts were dried over anhydrous Na₂SO₄, filtered, and concentrated to give a residue. The residue was triturated with petroleum ether/EtOAc (5/1, 1000 mL) to give a crude product which was triturated with DCM/MeOH (10/1, 500 mL) to give the product as a light yellow solid (180 g, 96%, 92% purity). MS ES+ m/z 293 [M+H]⁺.
Route 2: A mixture of 8-bromo-3,6-dimethyl-2-phenyl-chromen-4-one (50.0 g, 151.9 mmol), palladium acetate (0.34 g, 1.52 mmol), 1,3-bis(diphenylphosphino)propane (1.25 g, 3.04 mmol), triethylamine (46.17 g, 455.7 mmol), n-butyl vinyl ether (76 g, 759.5 mmol), and ethylene glycol (400 mL) was stirred at 100° C. for 7 h under a nitrogen atmosphere. The reaction was cooled to 30° C. and treated with 3 g of active carbon and stirred. The suspension was filtered through celite and the pH of the filtrate adjusted to 3-4 with HCl and stirred at 60° C. for 12 h. The reaction was cooled to 40° C. and a solid removed by filtration. The solids were slurried in 250 mL of THE and stirred at 60° C. for 5 h. The reaction was cooled to 30° C. and the product (40 g, 90%) removed by filtration and dried at 50° C. giving an off-white solid. MS ES+ m/z 293 [M+H]⁺. ¹H NMR (400 mHz, CDCl₃) δ ppm 2.21 (s, 3H), 2.52 (s, 3H), 2.73 (s, 3H), 7.55-7.58 (m, 3H), 7.67-7.70 (m, 2H), 7.97 (s, 1H), 8.26-8.27 (m, 1H).

The following compounds in Table 2 were made in a similar way as described for 8-acetyl-3,6-dimethyl-2-phenyl-chromen-4-one, Route 1.

TABLE 2

| | | | MS ES+ |
|---|---|---|---|
| Interm # | Chemical Name | Structure | m/z |
| 7 | 8-Acetyl-3,6-dimethyl-2- (3-pyridyl)chromen-4- one | 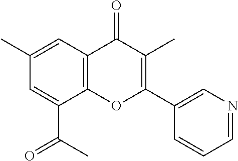 [SMILES: CC(=O)c1cc(C)cc2c(=O)c(C)c(-c3cccnc3)oc12] | |
| 3A | 8-Acetyl-2-ethylsulfanyl- 3,6-dimethyl-chromen-4- one | 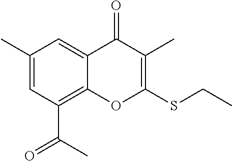 [SMILES: CCSc1oc2c(C(C)=O)cc(C)cc2c(=O)c1C] | 277 [M + H]⁺ |
| 8B | 8-Acetyl-2-ethylsulfanyl- 6-methyl-chromen-4-one | 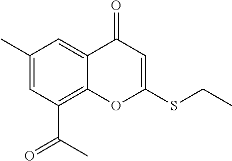 [SMILES: CCSc1cc(=O)c2cc(C)cc(C(C)=O)c2o1] | 263 [M + H]⁺ |
| 9B | 8-Acetyl-7-chloro-3,6- dimethyl-2-(3- pyridyl)chromen-4-one | 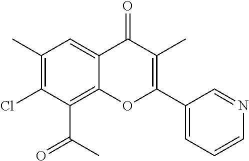 [SMILES: CC(=O)c1c(Cl)c(C)cc2c(=O)c(C)c(-c3cccnc3)oc12] | 328 [M + H]⁺ |

### Intermediate 4A: 2-Ethylsulfanyl-8-[(15)-1-hydroxyethyl]-3,6-dimethyl-chromen-4-one

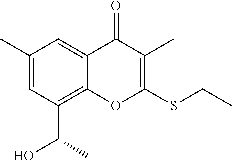 [SMILES: CCSc1oc2c([C@H](C)O)cc(C)cc2c(=O)c1C]

Formic acid (9.99 g, 217 mmol) and triethylamine (14.6 g, 145 mmol) were dissolved in DCM (500 mL) and cooled to 0° C. When cold, 8-acetyl-2-ethylsulfanyl-3,6-dimethyl-chromen-4-one (20.0 g, 72.4 mmol) was added and stirred for 1 to 2 min to obtain a homogeneous solution. RuCl[(R,R)-Tsdpen(mesitylene) (CAS 174813-82-2, 2.50 g, 90%, 3.62 mmol) was added and allowed to stir for 10 min before removing the cooling bath and was stirred at rt overnight. The reaction was washed with saturated aqueous NaHCO₃. The aqueous layer was extracted with IPA/CHCl₃ (1:3, v:v) three times. The organic layers were combined, dried over MgSO₄, filtered, and concentrated under reduced pressure. The crude residue was suspended in DCM (500 mL) and transferred to a large Erlenmeyer flask equipped with a stir bar. The suspension was diluted with DCM/heptane (5:1) to a total volume of 1500 mL. The contents of the flask were stirred at 200 rpm and warmed to 40° C. When warm, DCM was added until most of the solids had dissolved (75 mL). The flask was allowed to cool to rt and then placed into a freezer (−20° C.) overnight. The solids were removed by filtration, dissolved fully in DCM, filtered, and then the filtrate was concentrated under reduced pressure to give the title compound (7.41 g, 37%) as a white solid. ES-MS m/z 279 (M+H).

### Intermediate 1C: 2-Ethylsulfanyl-8-[(1R)-1-hydroxyethyl]-3,6-dimethyl-chromen-4-one

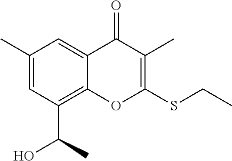 [SMILES: CCSc1oc2c([C@@H](C)O)cc(C)cc2c(=O)c1C]

To a stirred solution of 8-acetyl-2-ethylsulfanyl-3,6-dimethyl-chromen-4-one (12.0 g, 43.4 mmol) in MeOH (150 mL) was added sodium borohydride (2.46 g, 65.1 mmol) at rt under nitrogen. The mixture was cooled to 0° C., quenched with aqueous HCl (1M; 10 mL), and the pH adjusted to ˜7 with aqueous sodium hydroxide. The resulting mixture was diluted with water (100 mL) and extracted with EtOAc (2×500 mL). The combined organic layers were washed with saturated aqueous sodium chloride (100 mL), dried over sodium sulfate, and filtered. The filtrate was concentrated under reduced pressure and the residue was purified by reversed phase chromatography eluted with 60% to 70% ACN in 0.1% aqueous formic acid to give the racemic product (10 g, 83%) as a white solid. MS ES+ m/z 279 [M+H]⁺.

The racemic product was purified by Prep-SFC [Opti-Chiral C9-5, 30×250 mm; 15% MeOH in CO₂; 100 mL/min] to give two enantiomers as white solids. The first eluting enantiomer (4.7 g, 47%) was the title compound; the second eluting enantiomer (4.1 g, 41%) matched Intermediate 4A. MS ES+ m/z 279 [M+H]+, for both.

### Intermediate 10B: 2-Ethylsulfanyl-8-[(1S)-1-hydroxyethyl]-6-methyl-chromen-4-one

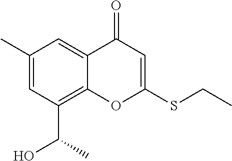 [SMILES: CCSc1cc(=O)c2cc(C)cc([C@H](C)O)c2o1]

8-Acetyl-2-ethylsulfanyl-6-methyl-chromen-4-one was used in a manner analogous to the method of preparation of Intermediate 4A to afford the title compound (1.24 g, 63%). MS ES+ m z 265 [M+H]⁺.

### Intermediate 2C: 3-Chloro-2-ethylsulfanyl-8-[(1S)-1-hydroxyethyl]-6-methyl-chromen-4-one

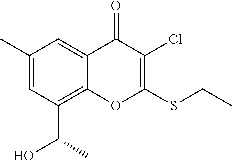 [SMILES: CCSc1oc2c([C@H](C)O)cc(C)cc2c(=O)c1Cl]

2-Ethylsulfanyl-8-[(1S)-1-hydroxyethyl]-6-methyl-chromen-4-one (0.105 g, 0.397 mmol), N-chlorosuccinimide (0.066 g, 0.497 mmol), and benzoyl peroxide (0.096 g, 0.397 mmol) were combined in acetonitrile (7 mL) and stirred at 25° C. for 2 h. The reaction mixture was concentrated under reduced pressure. The residue was purified by silica gel chromatography eluted with 10% to 70% EtOAc in heptane to give the title compound (0.100 g, 84%) as a white solid. MS ES+ m/z 299 [M+H]⁺.

### Intermediate 8: 8-[(1S)-1-Hydroxyethyl]-3,6-dimethyl-2-phenyl-chromen-4-one

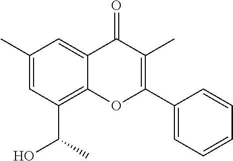 [SMILES: Cc1cc([C@H](C)O)c2oc(-c3ccccc3)c(C)c(=O)c2c1]

A flask equipped with overhead stirring and a temperature probe was charged with 8-acetyl-3,6-dimethyl-2-phenyl-chromen-4-one (11.0 g, 37.25 mmol) and chloroform (200 mL). The stirring slurry was treated with formic acid (5.14 g, 111.76 mmol) and cooled to −10° C. in an ice bath. The cold solution was slowly treated with 1,8-diazabicyclo[5.4.0]undec-7-ene (17.01 g, 111.76 mmol) maintaining the temperature below 25° C. The reaction was removed from the cooling bath and treated with RuCl(p-cymene)[(S,S)-Ts-DPEN] (CAS 192139-90-5, 0.66 g, 1.12 mmol). The reaction was stirred at 45° C. for 16 h. The reaction was transferred to a separatory funnel and washed with 2M aqueous HCl (2×50 mL). The organic layer was concentrated under vacuum at 45-50° C. to 50 to 100 mL. Diluted with ACN (150 mL) and concentrated under vacuum at 45-50° C. to 50 mL. The solvent swap was done again until the amount of solvent was 50 mL. The material was cooled to rt over 1-2 h and the slurry aged for 4 h. The product (10.10 g, 92%) was collected by filtration, washed with ACN (25 mL), washed with heptane (50 mL), and dried under vacuum at 45° C.

### Intermediate 9: 8-[(1R)-1-Hydroxyethyl]-3,6-dimethyl-2-phenyl-chromen-4-one

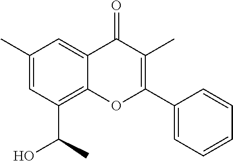 [SMILES: Cc1cc([C@@H](C)O)c2oc(-c3ccccc3)c(C)c(=O)c2c1]

A flask equipped with overhead stirring, condenser, and temperature probe was charged with 8-acetyl-3,6-dimethyl-2-phenyl-chromen-4-one (10 g, 34.2 mmol) and RuCl(p-cymene)[(R,R)-TsDPEN] (CAS 192139-92-7, 0.65 g, 1.03 mmol). Added 50 mL of methanol and started stirring. The reaction was cooled to 10° C. and treated slowly with 1,8-diazabicyclo[5.4.0]undec-7-ene (15.62 g, 102.62 mmol) keeping the temperature below 25° C. After addition was complete, cooled the reaction back down to 10° C. and treated the reaction in portions with formic acid (4.72 g, 102.62 mmol) maintaining the temperature below 15° C. After addition, the reaction was stirred at 55° C. for about 3 h. The reaction was cooled to 20° C. and treated with 4 M aqueous HCl (50 mL) over 1 h and the resulting slurry stirred overnight at rt. The product (9.35 g, 95%) was isolated by filtration, washed with water, and dried under vacuum at 45° C.

The following compound in Table 3 was made in a similar way as described for 8-[(1R)-1-hydroxyethyl]-3,6-dimethyl-2-phenyl-chromen-4-one.

TABLE 3

| | | | MS ES+ |
|---|---|---|---|
| Interm # | Chemical Name | Structure | m/z |
| 10 | 8-[(1R)-1- Hydroxyethyl]-3,6- dimethyl-2-(3- pyridyl)chromen-4-one | 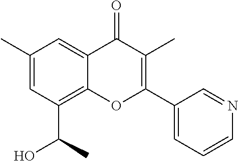 [SMILES: Cc1cc([C@@H](C)O)c2oc(-c3cccnc3)c(C)c(=O)c2c1] | 296 [M + H]⁺ |

### Intermediate 11B: 7-Chloro-8-(1-hydroxyethyl)-3,6-dimethyl-2-(3-pyridyl)chromen-4-one

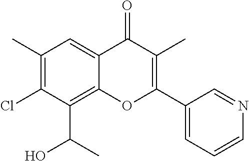 [SMILES: Cc1cc2c(=O)c(C)c(-c3cccnc3)oc2c(C(C)O)c1Cl]

A 0° C. solution of 8-acetyl-7-chloro-3,6-dimethyl-2-(3-pyridyl)chromen-4-one (0.550 g, 1.68 mmol) in DCM (10 mL) and MeOH (10 mL) was treated with sodium borohydride (0.064 g, 1.68 mmol) and stirred for 2 h. The reaction mixture was diluted with DCM (120 mL) and water (75 mL). The layers were separated and the aqueous layer was extracted with DCM (2×). The combined organic layers were dried over MgSO₄, filtered, and concentrated under reduced pressure. The residue was purified by silica gel chromatography eluted with 0% to 100% EtOAc in heptane to give the title compound (0.23 g, 42%). MS ES+ m/z 330 [M+H]⁺.

### Intermediate 12B: 2-Benzylsulfanyl-6-chloro-3-fluoro-pyridine

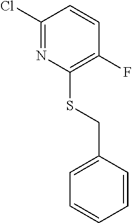 [SMILES: Fc1ccc(Cl)nc1SCc1ccccc1]

A 0° C. solution of phenylmethanethiol (0.88 mL, 7.57 mmol) and potassium hydroxide (0.411 g, 7.32 mmol) in EtOH (5 mL) was treated with a solution of 2,6-dichloro-3-fluoropyridine (1.00 g, 6.02 mmol) in EtOH (5 mL), then additional EtOH (2 mL). The ice bath was removed, and the reaction mixture was stirred for 20 h. The mixture was diluted with EtOAc, 1M aqueous NaOH, and saturated aqueous sodium chloride. The organic layer was removed. The aqueous layer was extracted with DCM (2×). The combined organic layers were dried over MgSO₄, filtered, and concentrated under reduced pressure. The residue was purified by silica gel chromatography eluted with 0% to 50% MTBE in heptane to give the title compound (0.84 g, 51%) as a colorless oil. MS ES+ m/z 254 [M+H]⁺.

The following compounds in Table 4 were made in a similar way as described for 2-benzylsulfanyl-6-chloro-3-fluoro-pyridine. Various methods were used to purify the compounds, which would be apparent to one skilled in the art.

TABLE 4

| | | | MS ES+ |
|---|---|---|---|
| Interm # | Chemical Name | Structure | m/z |
| 3C | 2-Benzylsulfanyl-6- chloro-3-fluoro- pyridine | 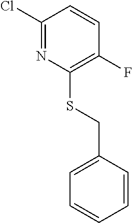 [SMILES: Fc1ccc(Cl)nc1SCc1ccccc1] | 254 [M + H]⁺ |
| 4C | 2-Benzylsulfanyl-3- fluoro-6-methyl- pyridine | 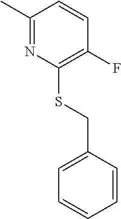 [SMILES: Cc1ccc(F)c(SCc2ccccc2)n1] | 234 [M + H]⁺ |
| 5C | 2-Benzylsulfanyl-6- fluoro-pyridin-3-ol | 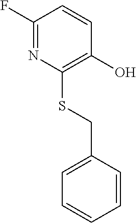 [SMILES: Oc1ccc(F)nc1SCc1ccccc1] | 236 [M + H]⁺ |
| 6C | 2-Benzylsulfanyl-3- bromo-6-methoxy- pyridine | 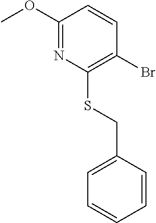 [SMILES: COc1ccc(Br)c(SCc2ccccc2)n1] | 310 [M + H]⁺ |

### Intermediate 7C: 3-Benzyloxy-2-benzylsulfanyl-6-fluoro-pyridine

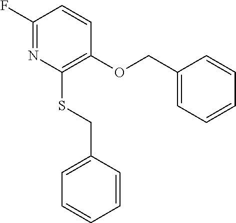 [SMILES: Fc1ccc(OCc2ccccc2)c(SCc2ccccc2)n1]

To a stirred solution of 2-benzylsulfanyl-6-fluoro-pyridin-3-ol (1.00 g, 4.25 mmol) in acetonitrile (5 mL) was added potassium carbonate (1.76 g, 12.8 mmol) in portions at room temperature, followed by dropwise addition of (bromomethyl)benzene (0.80 g, 4.68 mmol). The resulting mixture was heated at 60° C. for 4 h, then cooled to rt and filtered. The filtrate was concentrated under reduced pressure and the residue was purified by silica gel chromatography eluted with 0-100% EtOAc in heptane to give the title compound (1.12 g, 81%). MS ES+ m/z 326 [M+H]⁺.

### Intermediate 13B: 6-Chloro-3-fluoro-pyridine-2-sulfonamide

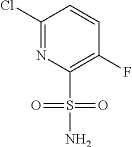 [SMILES: NS(=O)(=O)c1nc(Cl)ccc1F]

A 0° C. solution of 2-benzylsulfanyl-6-chloro-3-fluoro-pyridine (12.77 g, 80%, 40.3 mmol) in DCM (70 mL), aqueous HCl (12M, 21 mL), and water (35 mL) was treated with aqueous sodium hypochlorite (7.5 wt %, 140 mL, 169 mmol), dropwise over 20 min. The reaction mixture was stirred for 20 min, then extracted with DCM (3×50 mL). The combined organic layers were cooled to 0° C. and treated with ammonium hydroxide (29 wt %, 30 mL, 242 mmol). The reaction mixture was stirred for 10 min, then concentrated under reduced pressure. The residue was dissolved in a mixture of EtOAc, DCM, and EtOH, then filtered. The filtrate was concentrated under reduced pressure. The residue was suspended in DCM and filtered to give the title compound (5.6 g, ˜90% purity) as a white solid. MS ES+ m/z 211 [M+H]⁺.

The following compounds in Table 5 were made in a similar way as described for 6-chloro-3-fluoro-pyridine-2-sulfonamide. Various methods were used to purify the compounds, which would be apparent to one skilled in the art.

TABLE 5

| | | | MS ES+ |
|---|---|---|---|
| Interm # | Chemical Name | Structure | m/z |
| 8C | 6-Chloro-3-fluoro-N- methoxy-pyridine-2- sulfonamide | 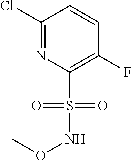 | 241 [M + H]⁺ |
| 9C | N-tert-Butyl-3-fluoro-6- methyl-pyridine-2- sulfonamide | 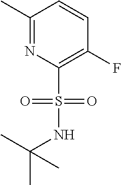 [SMILES: Cc1ccc(F)c(S(=O)(=O)NC(C)(C)C)n1] | 247 [M + H]⁺ |
| 10C | 2,3-Difluoro-6-methoxy- benzenesulfonamide | 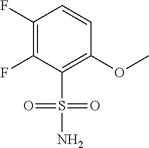 | 222 [M − H]⁻ |
| 11C | 3-Benzyloxy-6-fluoro- pyridine-2-sulfonamide | 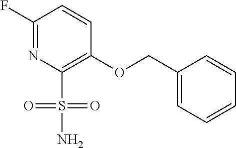 [SMILES: NS(=O)(=O)c1nc(F)ccc1OCc1ccccc1] | 283 [M + H]⁺ |
| 12C | 3-Bromo-N-tert-butyl-6- methoxy-pyridine-2- sulfonamide | 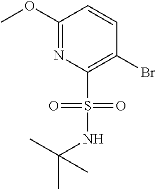 [SMILES: COc1ccc(Br)c(S(=O)(=O)NC(C)(C)C)n1] | 323 [M + H]⁺ |

### Intermediate 14B: 6-Chloro-3-hydroxy-pyridine-2-sulfonamide

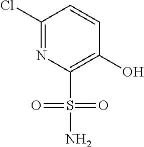 [SMILES: NS(=O)(=O)c1nc(Cl)ccc1O]

A mixture of 6-chloro-3-fluoro-pyridine-2-sulfonamide (4.93 g, 23.4 mmol) and potassium hydroxide (13.1 g, 234 mmol) in water (35 mL) was heated at 110° C. for 2.5 h. The reaction mixture was cooled to rt and the pH was adjusted to ˜4-5 with aqueous HCl (12M, ˜16 mL) and 10% aqueous citric acid (˜15 mL). The mixture was filtered. The filtrate was extracted with isopropanol:chloroform (1:3) (5×80 mL). The combined organic layers were washed with saturated aqueous sodium chloride, dried over Na₂SO₄, filtered, and concentrated under reduced pressure. The residue was purified by silica gel chromatography eluted with 0% to 50% EtOAc in heptane to give the title compound (1.30 g, 27%). MS ES+ m/z 209 [M+H]⁺.

### Intermediate 13C: N-tert-Butyl-3-hydroxy-6-methoxy-pyridine-2-sulfonamide

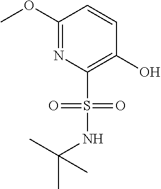 [SMILES: COc1ccc(O)c(S(=O)(=O)NC(C)(C)C)n1]

To a stirred solution of cuprous iodide (0.018 g, 0.093 mmol) and 2-methyl-quinolin-8-ol (0.015 g, 0.093 mmol) in DMSO (2 mL) was added a mixture of 3-bromo-N-tert-butyl-6-methoxy-pyridine-2-sulfonamide (0.300 g, 0.928 mmol) and tetrabutylazanium oxidanide (40 wt %; 2.03 mL, 2.78 mmol) in water (3 mL) dropwise at rt. The reaction mixture was stirred at 130° C. for 2 h, then cooled to rt and filtered. The filtrate was concentrated under reduced pressure. The residue was purified by silica gel chromatography eluted with 0-100% EtOAc in heptane to give the title compound (0.145 g, 60%). MS ES+ m/z 261 [M+H]⁺.

### Intermediate 14C: 3-Fluoro-6-methyl-pyridine-2-sulfonamide

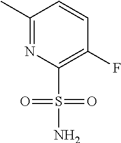

To a stirred solution of N-tert-butyl-3-fluoro-6-methyl-pyridine-2-sulfonamide (1.50 g, 6.09 mmol) in DCM (10 mL) was added TFA (10 mL) dropwise at rt under nitrogen. The resulting mixture was stirred overnight at 50° C., cooled to rt, and concentrated under reduced pressure. The material was diluted with DCM (10 mL) and the pH adjusted to ˜8 with triethylamine. The residue was purified by silica gel chromatography eluted with 40% EtOAc in petroleum ether to give the title compound (0.57 g, 49%) as a white solid. MS ES+ m/z 191 [M+H]⁺.

### Intermediate 15C: 3-Hydroxy-6-methoxy-pyridine-2-sulfonamide

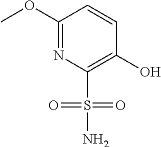

N-tert-Butyl-3-hydroxy-6-methoxy-pyridine-2-sulfonamide was used in a manner analogous to the method of preparation of Intermediate 14C to afford the title compound (0.098 g, 89%). MS ES+ m/z 205 [M+H]⁺.

### Intermediate 16C: 2,3-Difluoro-6-hydroxy-benzenesulfonamide

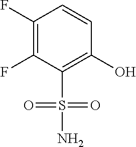 [SMILES: NS(=O)(=O)c1c(O)ccc(F)c1F]

A solution of 2,3-difluoro-6-methoxy-benzenesulfonamide (0.465 g, 2.08 mmol) in DCM (20 mL) was treated with tribromoborane (1M; 10.4 mL, 10.4 mmol) dropwise at 0° C. The resulting mixture was stirred at 0° C. for 2 h. The reaction was concentrated under reduced pressure and the residue was purified by reversed phase chromatography eluted with 5% to 40% ACN in 10 mM aqueous NH₄HCO₃ with 5% MeOH to give the title compound (0.34 g, 77%) as a white solid. MS ES+ m/z 208 [M−H]⁻.

### Intermediate 17C: 6-Fluoro-3-hydroxy-pyridine-2-sulfonamide

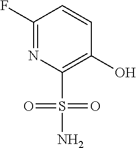 [SMILES: NS(=O)(=O)c1nc(F)ccc1O]

A solution of 3-benzyloxy-6-fluoro-pyridine-2-sulfonamide (0.690 g, 2.44 mmol) in EtOH (10 mL) was sparged with argon for 10 min, then Pd/C (10 wt %; 0.260 g) was added. The reaction vial was flushed with hydrogen and stirred vigorously under a hydrogen atmosphere (balloon) for 2 h. The reaction was filtered through diatomaceous earth. The filtrate was concentrated under reduced pressure to give the crude title compound. MS ES+ m/z 193 [M+H]⁺.

### Intermediate 18C: 5-Bromo-2-(fluoromethyl)indazole

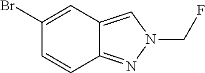 [SMILES: FCn1cc2cc(Br)ccc2n1]

To a stirred mixture of 5-bromo-2H-indazole (5.00 g, 25.4 mmol) and fluoromethyl 4-methylbenzenesulfonate (5.70 g, 27.9 mmol) in NMP (50 mL) was added cesium carbonate (9.92 g, 30.5 mmol) in portions at rt under nitrogen. The resulting mixture was stirred at 60° C. for 1 h, then cooled to rt. The reaction was diluted with water (60 mL) and extracted with EtOAc (3×60 mL). The combined organic layers were washed with saturated aqueous sodium chloride (6×30 mL), dried over sodium sulfate, and filtered. The filtrate was concentrated under reduced pressure. The residue was purified by silica gel chromatography eluted with 15-50% EtOAc in petroleum ether to give the title compound (1.10 g, 19%) as a yellow solid. MS ES+ m/z 229 [M+H]⁺.

### Intermediate 19C: tert-Butyl-dimethyl-[2-[5-(4,4,5,5-tetramethyl-1,3,2-dioxaborolan-2-yl)indazol-2-yl]ethoxy]silane

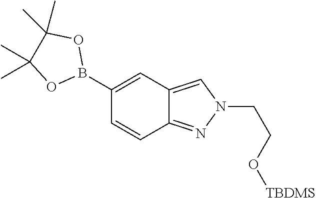

5-(4,4,5,5-tetramethyl-1,3,2-dioxaborolan-2-yl)-1H-indazole (3.00 g, 12.3 mmol) and (2-bromoethoxy)(tert-butyl)dimethylsilane (14.70 g, 61.5 mmol) were combined with diisopropylethylamine (3.18 g, 24.6 mmol) and N,N-dimethylpyridin-4-amine (1.50 g, 12.3 mmol) in DMF (20 mL). The mixture was stirred at 110° C. for 48 h, then concentrated under reduced pressure. The residue was purified by silica gel chromatography eluted with 10-100% EtOAc in heptane to give the title compound (1.10 g, 22%). MS ES+ m/z 403 [M+H]⁺.

The following compound in Table 6 was made in a similar way as described for tert-butyl-dimethyl-[2-[5-(4,4,5,5-tetramethyl-1,3,2-dioxaborolan-2-yl)indazol-2-yl]ethoxy]silane. Various methods were used to purify the compound, which would be apparent to one skilled in the art.

TABLE 6

| | | | MS ES+ |
|---|---|---|---|
| Interm # | Chemical Name | Structure | m/z |
| 20C | 1-(3,3- Difluoropropyl)-4- (4,4,5,5-tetramethyl- 1,3,2-dioxaborolan- 2-yl)pyrazole | 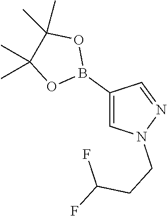 [SMILES: CC1(C)OB(c2cnn(CCC(F)F)c2)OC1(C)C] | 273 [M + H]⁺ |

### Intermediates 21C and 22C: 5-Chloro-1-methyl-pyrazolo[4,3-b]pyridine and 5-Chloro-2-methyl-pyrazolo[4,3-b]pyridine

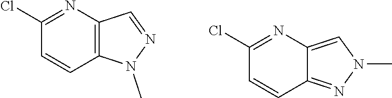

To a stirred mixture of 5-chloro-1H-pyrazolo[4,3-b]pyridine (3.20 g, 20.8 mmol) and iodomethane (4.44 g, 31.3 mmol) in THE (30 mL) was added sodium hydride (60%; 1.00 g, 25.0 mmol) in portions at 0° C. under nitrogen. The resulting mixture was stirred at rt for 1 h. The reaction was quenched with water (30 mL) and extracted with EtOAc (3×80 mL). The combined organic layers were washed with saturated aqueous sodium chloride (3×30 mL), dried over sodium sulfate, and filtered. The filtrate was concentrated under reduced pressure. The residue was purified by silica gel chromatography eluted with 10% EtOAc in petroleum ether to give 5-chloro-1-methyl-pyrazolo[4,3-b]pyridine (1.84 g, 53%) and further eluted with 30% EtOAc in petroleum ether to give 5-chloro-2-methyl-pyrazolo[4,3-b]pyridine (1.34 g, 38%). MS ES+ m/z 168 [M+H]+, for both.

The following compounds in Table 7 were made in a similar way as described for 5-chloro-1-methyl-pyrazolo[4,3-b]pyridine and 5-chloro-2-methyl-pyrazolo[4,3-b]pyridine. Various methods were used to purify the compounds, which would be apparent to one skilled in the art.

TABLE 7

| | | | MS ES+ |
|---|---|---|---|
| Interm # | Chemical Name | Structure | m/z |
| 23C | 5-Bromo-1-ethyl- indazole | 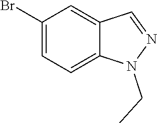 | |
| 24C | 5-Bromo-2-ethyl- indazole | 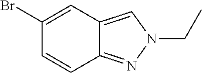 | 225 [M + H]⁺ |
| 25C | 4-Bromo-1-(2- oxabicyclo[2.1.1]hexan- 4-ylmethyl)pyrazole | 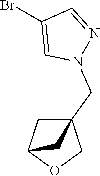 [SMILES: Brc1cnn(C[C@]23CO[C@H](C2)C3)c1] | 243 [M + H]⁺ |
| 26C | 4-Bromo-1-(2- oxabicyclo[2.1.1]hexan- 1-ylmethyl)pyrazole | 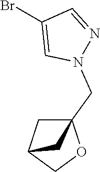 [SMILES: Brc1cnn(C[C@]23C[C@H](CO2)C3)c1] | 243 [M + H]⁺ |

### Intermediate 27C: Trimethyl-(1-methylpyrazolo[3,4-b]pyridin-6-yl)stannane

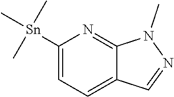

To a stirred solution of 6-chloro-1-methylpyrazolo[3.4-b]pyridine (1.10 g, 6.56 mmol) and hexamethyldistannane (2.58 g, 7.88 mmol) in 1,4-dioxane (10 mL) was added tetrakis(triphenylphosphine)palladium(0) (0.758 g, 0.656 mmol) in portions at rt under nitrogen. The resulting mixture was stirred at 100° C. for 2 h. The reaction was cooled to rt, quenched with saturated aqueous cesium fluoride (100 mL), and extracted with EtOAc (2×10⁰ mL). The combined organic layers were washed with saturated aqueous sodium chloride (2×80 mL), dried over sodium sulfate, and filtered. The filtrate was concentrated under reduced pressure to give the title compound (2.1 g, crude) as a yellow oil. MS ES+ m/z 298 [M+H]⁺.

The following compounds in Table 8 were made in a similar way as described for trimethyl-(1-methylpyrazolo[3,4-b]pyridin-6-yl)stannane. Alternate reaction conditions included tributyl tinchloride with n-butyllithium in THF. Various methods were used to purify the compounds, which would be apparent to one skilled in the art.

TABLE 8

| | | | MS ES+ |
|---|---|---|---|
| Interm # | Chemical Name | Structure | m/z |
| 28C | Trimethyl-(2- methylpyrazolo[4,3- b]pyridin-5-yl)stannane | 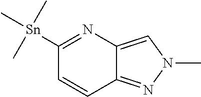 | 298 [M + H]⁺ |
| 29C | 2-Methyl-4- tributylstannyl-pyrazole- 3-carbonitrile | 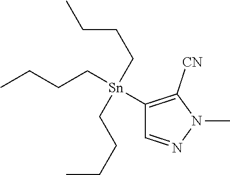 [SMILES: CCC[CH2][Sn]([CH2]CCC)([CH2]CCC)[c]1cnn(C)c1C#N] | 398 [M + H]⁺ |
| 30C | 6-Trimethylstannyl imidazo[1,2-a]pyridine- 2-carbonitrile | 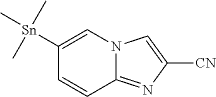 | 308 [M + H]⁺ |
| 31C | (2-Ethylindazol-5-yl)- trimethyl-stannane | 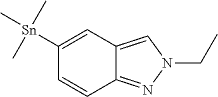 | 311 [M + H]⁺ |
| 32C | Tributyl(2,3- dihydropyrazolo[5,1- b]oxazol-7-yl)stannane | 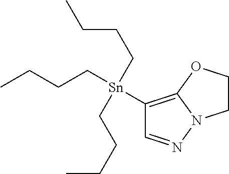 [SMILES: CCC[CH2][Sn]([CH2]CCC)([CH2]CCC)[c]1cnn2c1OCC2] | 401 [M + H]⁺ |
| 33C | Tributyl(6,8-dihydro-5H- imidazo[2,1- c][1,4]oxazin-3- yl)stannane | 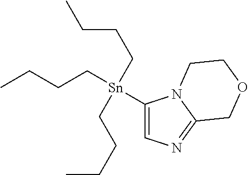 [SMILES: CCC[CH2][Sn]([CH2]CCC)([CH2]CCC)[c]1cnc2n1CCOC2] | 415 [M + H]⁺ |
| 34C | Tributyl(6,8-dihydro-5H- imidazo[2,1- c][1,4]oxazin-2- yl)stannane | 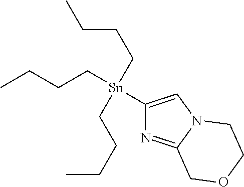 [SMILES: CCC[CH2][Sn]([CH2]CCC)([CH2]CCC)[c]1cn2c(n1)COCC2] | 415 [M + H]⁺ |

### Intermediate 35C: 2-Methyl-6-(4,4,5,5-tetramethyl-1,3,2-dioxaborolan-2-yl)oxazolo[5,4-b]pyridine

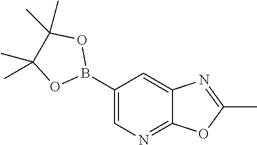

6-Bromo-2-methyl-oxazolo[5,4-b]pyridine (0.200 g, 0.939 mmol), potassium acetate (0.184 g, 1.88 mmol), 1,1′-bis(diphenylphosphino)ferrocenedichloro palladium(II) (0.069 g, 0.094 mmol), and bis(pinacolato)diboron (0.358 g, 1.41 mmol) were combined in 1,4-dioxane (4 mL). The reaction was purged with nitrogen for 5 min, stirred at 80° C. for 14.5 h, then cooled to rt. The mixture was diluted with ethanol and filtered through diatomaceous earth. The filtrate was concentrated under reduced pressure to give the crude title compound. MS ES+ m/z 261 [M+H]⁺.

The following compounds in Table 9 were made in a similar way as described for 2-methyl-6-(4,4,5,5-tetramethyl-1,3,2-dioxaborolan-2-yl)oxazolo[5,4-b]pyri dine. Alternate reaction conditions included palladium(II)acetate and tricyclohexylphosphine. Various methods were used to purify the compounds, which would be apparent to one skilled in the art.

TABLE 9

| | | | MS ES+ |
|---|---|---|---|
| Interm # | Chemical Name | Structure | m/z |
| 36C | 7-(4,4,5,5-Trimethyl- 1,3,2-dioxaborolan-2-yl)- 2,3-dihydropyrazolo[5,1- b]oxazole | 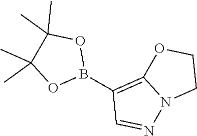 [SMILES: CC1(C)OB(c2cnn3c2OCC3)OC1(C)C] | 237 [M + H]⁺ |
| 37C | 8-Fluoro-2-methyl-6- (4,4,5,5-tetramethyl- 1,3,2-dioxaborolan-2- yl)imidazo[1,2-a]pyridine | 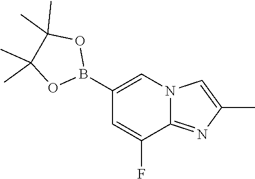 | 277 [M + H]⁺ |
| 38C | 6-Fluoro-2-methyl-5- (4,4,5,5-tetramethyl- 1,3,2-dioxaborolan-2- yl)indazole | 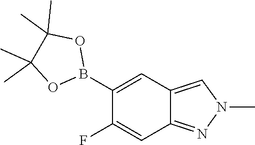 | 277 [M + H]⁺ |
| 39C | 4-Fluoro-2-methyl-5- (4,4,5,5-tetramethyl- 1,3,2-dioxaborolan-2- yl)indazole | 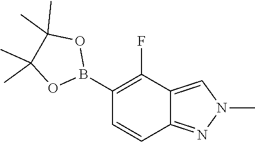 | 277 [M + H]⁺ |
| 40C | 4,4-Dimethyl-6-(4,4,5,5- tetramethyl-1,3,2- dioxaborolan-2-yl)-1H- 3,1-benzoxazin-2-one | 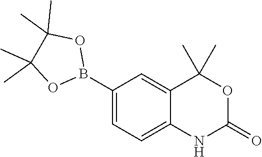 [SMILES: CC1(C)OC(=O)Nc2ccc(B3OC(C)(C)C(C)(C)O3)cc21] | 304 [M + H]⁺ |
| 41C | 3-Fluoro-2-methyl-5- (4,4,5,5-tetramethyl- 1,3,2-dioxaborolan-2- yl)indazole | 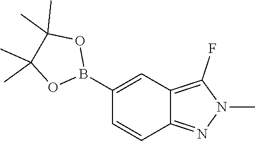 | 277 [M + H]⁺ |
| 42C | (7-Cyano-2-methyl- indazol-5-yl)boronic acid | 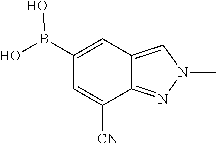 | 202 [M + H]⁺ |
| 43C | 6-(4,4,5,5-Tetramethyl- 1,3,2-dioxaborolan-2- yl)pyrazolo[1,5- a]pyridine | 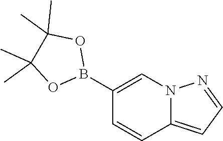 | 163 [M + H]⁺ |
| 44C | N-[6-(4,4,5,5- Tetramethyl-1,3,2- dioxaborolan-2-yl)-1,3- benzothiazol-2- yl]acetamide | 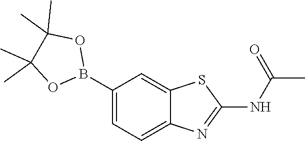 [SMILES: CC(=O)Nc1nc2ccc(B3OC(C)(C)C(C)(C)O3)cc2s1] | 319 [M + H]⁺ |
| 45C | 2-Methoxy-6-(4,4,5,5- tetramethyl-1,3,2- dioxaborolan-2- yl)quinoline | 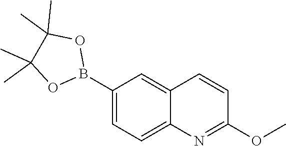 | 286 [M + H]⁺ |
| 46C | 3,3-Dimethyl-7-(4,4,5,5- tetramethyl-1,3,2- dioxaborolan-2-yl)-2H- pyrazolo[5,1-b]oxazole | 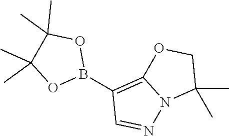 | 265 [M + H]⁺ |
| 47C | 2-(Fluoromethyl)-5- (4,4,5,5-tetramethyl- 1,3,2-dioxaborolan-2- yl)indazole | 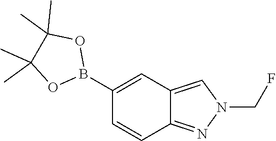 [SMILES: CC1(C)OB(c2ccc3nn(CF)cc3c2)OC1(C)C] | 277 [M + H]⁺ |
| 48C | 2-Methyl-5-(4,4,5,5- tetramethyl-1,3,2- dioxaborolan-2- yl)pyrazolo[1,5- a]pyridine | 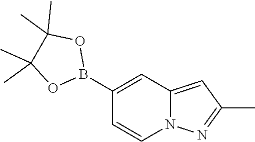 | 259 [M + H]⁺ |
| 49C | 2-Ethyl-6-(4,4,5,5- tetramethyl-1,3,2- dioxaborolan-2-yl)-1,3- benzothiazole | 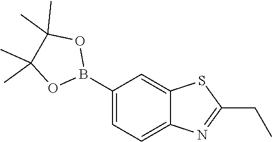 [SMILES: CCc1nc2ccc(B3OC(C)(C)C(C)(C)O3)cc2s1] | 290 [M + H]⁺ |
| 50C | 2-Ethyl-6-(4,4,5,5- tetramethyl-1,3,2- dioxaborolan-2-yl)-1,3- benzoxazole | 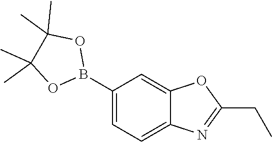 [SMILES: CCc1nc2ccc(B3OC(C)(C)C(C)(C)O3)cc2o1] | 274 [M + H]⁺ |
| 51C | 1-(2- Oxabicyclo[2.1.1]hexan- 4-ylmethyl)-4-(4,4,5,5- tetramethyl-1,3,2- dioxaborolan-2- yl)pyrazole | 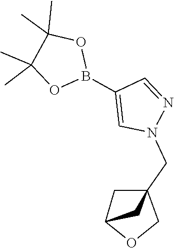 [SMILES: CC1(C)OB(c2cnn(C[C@]34CO[C@H](C3)C4)c2)OC1(C)C] | 291 [M + H]⁺ |
| 52C | 1-(2- Oxabicyclo[2.1.1]hexan- 1-ylmethyl)-4-(4,4,5,5- tetramethyl-1,3,2- dioxaborolan-2- yl)pyrazole | 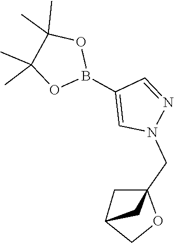 [SMILES: CC1(C)OB(c2cnn(C[C@]34C[C@H](CO3)C4)c2)OC1(C)C] | 291 [M + H]⁺ |
| 53C | 2-Methyl-6-(4,4,5,5- tetramethyl-1,3,2- dioxaborolan-2- yl)imidazo[1,2-a]pyridine | 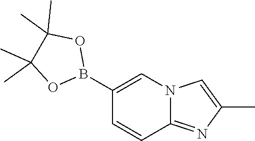 | 259 [M + H]⁺ |

### Intermediate 5A: 6-Chloro-3-[(1R)-1-(2-ethylsulfanyl-3,6-dimethyl-4-oxo-chromen-8-yl)ethoxy]pyridine-2-carbonitrile

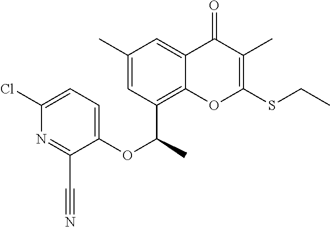 [SMILES: CCSc1oc2c([C@@H](C)Oc3ccc(Cl)nc3C#N)cc(C)cc2c(=O)c1C]

A solution of 2-ethylsulfanyl-8-[(1S)-1-hydroxyethyl]-3,6-dimethyl-chromen-4-one (1.0 g, 3.59 mmol), 6-chloro-3-hydroxy-pyridine-2-carbonitrile (0.83 g, 5.39 mmol), and triphenylphosphine (1.41 g, 5.39 mmol) in TI-W (12 mL) was treated with DIAD at 0′° C. After stirring at 0° C. for 1 h, the reaction was concentrated under reduced pressure and the residue purified by silica gel chromatography eluted with 000 to 10000 EtOAc in heptane to give the title compound (2.39 g, 60% purity, 96%) as a white foam. ES-MS m/z 415 (M+H).

The following compounds in Table 10 were made in a similar way as described for 6-chloro-3-[(1R)-1-(2-ethylsulfanyl-3,6-dimethyl-4-oxo-chromen-8-yl)ethoxy]pyridine-2-carbonitrile. Various methods were used to purify the compounds, which would be apparent to one skilled in the art.

TABLE 10

| | | | MS ES+ |
|---|---|---|---|
| Interm # | Chemical Name | Structure | m/z |
| 6A | 8-[(1R)-1-[(6-Chloro-2- fluoro-3- pyridyl)oxy]ethyl]-2- ethylsulfanyl-3,6- dimethyl-chromen-4-one | 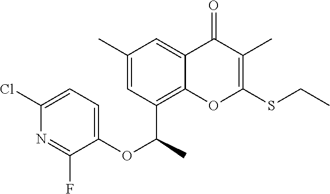 [SMILES: CCSc1oc2c([C@@H](C)Oc3ccc(Cl)nc3F)cc(C)cc2c(=O)c1C] | 408 [M + H]⁺ |
| 7A | Methyl 6-[(1R)-1-(2- ethylsulfanyl-3,6- dimethyl-4-oxo-chromen- 8-yl)ethoxy]-2,3-difluoro- benzoate | 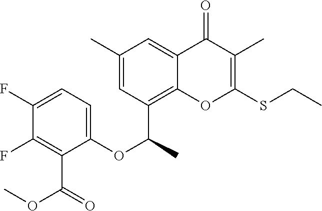 [SMILES: CCSc1oc2c([C@@H](C)Oc3ccc(F)c(F)c3C(=O)OC)cc(C)cc2c(=O)c1C] | 449 [M + H]⁺ |
| 15B | 3-[(1R)-1-(2- Ethylsulfanyl-3,6- dimethyl-4-oxo-chromen- 8-yl)ethoxy]-6-fluoro- pyridine-2-carbonitrile | 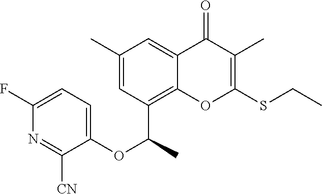 [SMILES: CCSc1oc2c([C@@H](C)Oc3ccc(F)nc3C#N)cc(C)cc2c(=O)c1C] | 399 [M + H]⁺ |
| 16B | methyl 6-Chloro-3-[(1R)- 1-(2-ethylsulfanyl-3,6- dimethyl-4-oxo-chromen- 8-yl)ethoxy]pyridine-2- carboxylate | 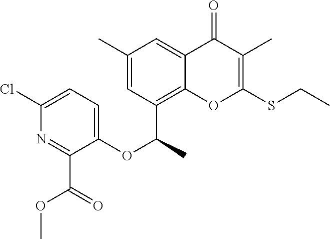 [SMILES: CCSc1oc2c([C@@H](C)Oc3ccc(Cl)nc3C(=O)OC)cc(C)cc2c(=O)c1C] | 448 [M + H]⁺ |
| 17B | 8-[(1R)-1-[(2-Chloro-3- pyridyl)oxy]ethyl]-2- ethylsulfanyl-3,6- dimethyl-chromen-4-one | 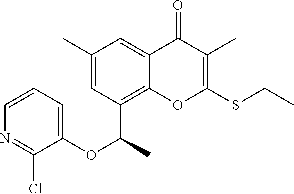 [SMILES: CCSc1oc2c([C@@H](C)Oc3cccnc3Cl)cc(C)cc2c(=O)c1C] | 390 [M + H]⁺ |
| 18B | 6-Chloro-3-[(1R)-1-(2- ethylsulfanyl-6-methyl-4- oxo-chromen-8- yl)ethoxy]pyridine-2- carbonitrile | 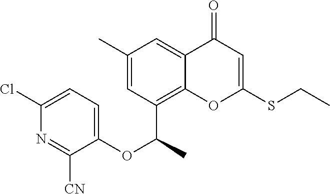 [SMILES: CCSc1cc(=O)c2cc(C)cc([C@@H](C)Oc3ccc(Cl)nc3C#N)c2o1] | 401 [M + H]⁺ |
| 19B | 3-[(1R)-1-(2- Ethylsulfanyl-6-methyl-4- oxo-chromen-8- yl)ethoxy]-6-fluoro- pyridine-2-carbonitrile | 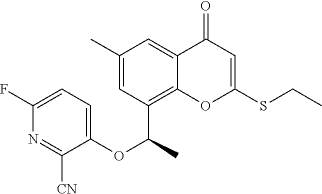 [SMILES: CCSc1cc(=O)c2cc(C)cc([C@@H](C)Oc3ccc(F)nc3C#N)c2o1] | 385 [M + H]⁺ |
| 54C | 6-Chloro-3-[(1R)-1-(2- ethylsulfanyl-3,6- dimethyl-4-oxo-chromen- 8-yl)ethoxy]pyridine-2- sulfonamide | 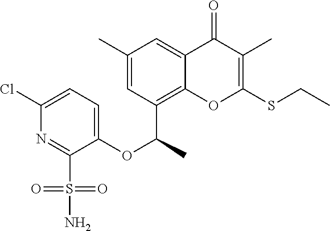 [SMILES: CCSc1oc2c([C@@H](C)Oc3ccc(Cl)nc3S(N)(=O)=O)cc(C)cc2c(=O)c1C] | 469 [M + H]⁺ |
| 55C | 3-[(1R)-1-(2- Ethylsulfanyl-3,6- dimethyl-4-oxo-chromen- 8-yl)ethoxy]pyridine-2- sulfonamide | 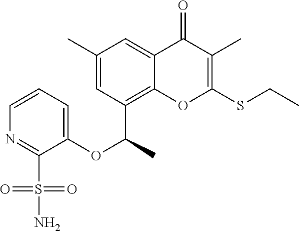 [SMILES: CCSc1oc2c([C@@H](C)Oc3cccnc3S(N)(=O)=O)cc(C)cc2c(=O)c1C] | 435 [M + H]⁺ |
| 56C | 3-[(1R)-1-(2- Ethylsulfanyl-6-methyl-6- oxo-chromen-8- yl)ethoxy]pyridine-2- sulfonamide | 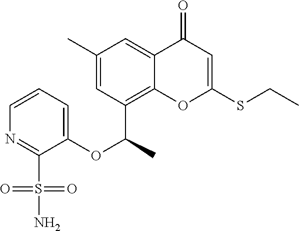 [SMILES: CCSc1cc(=O)c2cc(C)cc([C@@H](C)Oc3cccnc3S(N)(=O)=O)c2o1] | 421 [M + H]⁺ |
| 57C | 8-[(1R)-1-[(6-Chloro-2- methyl-3- pyridyl)oxy]ethyl]-2- ethylsulfanyl-3,6- dimethyl-chromen-4-one | 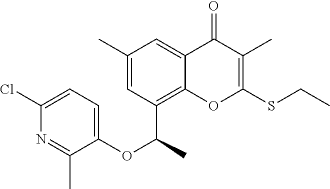 [SMILES: CCSc1oc2c([C@@H](C)Oc3ccc(Cl)nc3C)cc(C)cc2c(=O)c1C] | 404 [M + H]⁺ |
| 58C | 3-[(1R)-1-(2- Ethylsulfanyl-3,6- dimethyl-4-oxo-chromen- 8-yl)ethoxy]-6-fluoro- pyridine-2-sulfonamide | 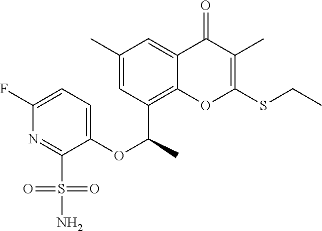 [SMILES: CCSc1oc2c([C@@H](C)Oc3ccc(F)nc3S(N)(=O)=O)cc(C)cc2c(=O)c1C] | 453 [M + H]⁺ |
| 59C | 1-[(1-Tetrahydropyran-2- yloxycyclopropyl)methyl]- 4-(4,4,5,5-tetramethyl- 1,3,2-dioxaborolan-2- yl)pyrazole | 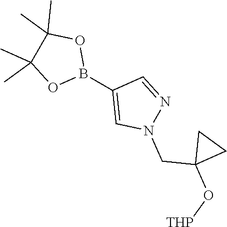 | 349 [M + H]⁺ |

### Intermediate 201B: 3-[(1R)-1-(2-Ethylsulfanyl-6-methyl-4-oxo-chromen-8-yl)ethoxy]-6-fluoro-pyridine-2-carboxamide

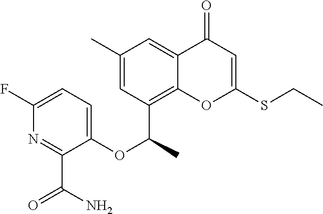 [SMILES: CCSc1cc(=O)c2cc(C)cc([C@@H](C)Oc3ccc(F)nc3C(N)=O)c2o1]

A mixture of 3-[(1R)-1-(2-ethylsulfanyl-6-methyl-4-oxo-chromen-8-yl)ethoxy]-6-fluoro-pyridine-2-carbonitrile (0.8 g, 2 mmol) and hydrido(dimethylphosphinous acid-kP) [hydrogen bis(dimethylphosphinito-kP)]platinum(II) (0.09 g, 0.2 mmol) in EtOH (5 mL) and water (5 mL) was stirred at 60° C. for 12 h. The reaction mixture was concentrated under reduced pressure and the residue purified by silica gel chromatography eluted with 000 to 6% MeOH in DCM to give the title compound (0.075 g, 50%) as a white solid after lyophilization. MS ES+ m/z 401 [M−H]⁻.

### Intermediate 60C: 6-Chloro-3-[(1R)-1-(2-ethylsulfanyl-6-methyl-4-oxo-chromen-8-yl)ethoxy]pyridine-2-carboxamide

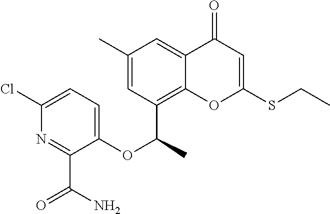 [SMILES: CCSc1cc(=O)c2cc(C)cc([C@@H](C)Oc3ccc(Cl)nc3C(N)=O)c2o1]

6-Chloro-3-[(1R)-1-(2-ethylsulfanyl-6-methyl-4-oxo-chromen-8-yl)ethoxy]pyridine-2-carbonitrile was used in a manner analogous to the method of preparation of Intermediate 20B to afford the title compound (0.122 g, 84%). MS ES+ m/z 417 [M−H]⁻.

### Intermediate 21B: 8-[(1R)-1-[(6-Chloro-2-morpholino-3-pyridyl)oxy]ethyl]-2-ethylsulfanyl-3,6-dimethyl-chromen-4-one

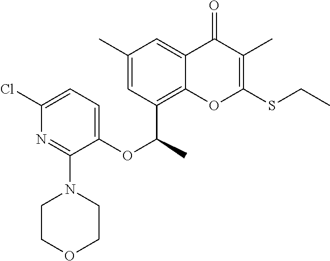 [SMILES: CCSc1oc2c([C@@H](C)Oc3ccc(Cl)nc3N3CCOCC3)cc(C)cc2c(=O)c1C]

A mixture of 8-[(1R)-1-[(6-chloro-2-fluoro-3-pyridyl)oxy]ethyl]-2-ethylsulfanyl-3,6-dimethyl-chromen-4-one (0.129 g, 0.316 mmol), DIPEA (0.22 mL, 1.27 mmol) and DMF (2 mL) was treated with morpholine (0.066 mL, 0.759 mmol). The reaction vessel was sealed and the mixture was stirred at rt for 18 h. The reaction mixture was heated to 60° C. for 1 h. Additional DIPEA (0.22 mL, 1.27 mmol) and morpholine (0.066 mL, 0.759 mmol) were added. The reaction mixture was heated to 60° C. for 4 h, then diluted with EtOAc and water. The layers were separated and the aqueous layer was extracted with EtOAc (3×). The combined organic layers were washed with saturated aqueous sodium chloride, then concentrated under reduced pressure. The residue was purified by silica gel chromatography eluted with 0% to 50% EtOAc in heptane to give the title compound (0.075 g, 50%) as a white solid. MS ES+ m/z 475 [M+H]⁺.

### Intermediate 22B: 2-Ethylsulfanyl-8-[(1R)-1-[[2-(2-fluorophenyl)-3-pyridyl]oxy]ethyl]-3,6-dimethyl-chromen-4-one

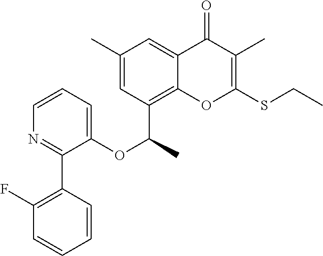 [SMILES: CCSc1oc2c([C@@H](C)Oc3cccnc3-c3ccccc3F)cc(C)cc2c(=O)c1C]

Combined 8-[(1R)-1-[(2-chloro-3-pyridyl)oxy]ethyl]-2-ethylsulfanyl-3,6-dimethyl-chromen-4-one (0.210 g, 0.539 mmol), (2-fluorophenyl)boronic acid (0.075 g, 0.539 mmol), K₂CO₃ (0.149 g, 1.08 mmol) and tetrakis(triphenylphosphine)palladium(0) (0.031 g, 0.027 mmol) with 1,4-dioxane (1.08 mL) and water (0.27 mL). The reaction was sparged for 5 min with argon, then stirred at 90° C. for 16 h. The reaction was concentrated under reduced pressure and purified by silica gel chromatography eluted with 0% to 60% EtOAc in heptane to give the title compound (0.24 g, quant) as a yellow oil. MS ES+ m/z 450 [M+H]⁺.

### Intermediate 23B: 8-[(1S)-1-[tert-Butyl(dimethyl)silyl]oxyethyl]-2-ethylsulfanyl-3,6-dimethyl-chromen-4-one

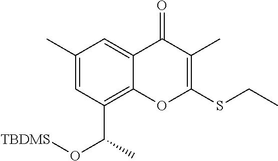

A solution of 2-ethylsulfanyl-8-[(1S)-1-hydroxyethyl]-3,6-dimethyl-chromen-4-one (0.500 g, 1.80 mmol) in DCM (10 mL) was treated with tert-butylchlorodimethylsilane (0.406 g, 2.69 mmol), 1,8-diazabicyclo[5.4.0]undec-7-ene (0.54 mL, 3.59 mmol) and 4-dimethylaminopyridine (0.219 g, 1.80 mmol). The reaction was stirred at rt for 3 h, then concentrated under reduced pressure. The residue was purified by silica gel chromatography eluted with 0% to 50% EtOAc in heptane to give the title compound (0.720 g, 97%) as a colorless oil. MS ES+ m/z 393 [M+H]⁺.

### Intermediate 24B: 8-[(1S)-1-[tert-Butyl(dimethyl)silyl]oxyethyl]-2-ethylsulfinyl-3,6-dimethyl-chromen-4-one

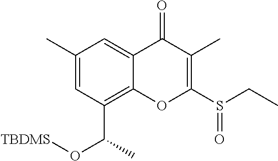

A solution of 8-[(1S)-1-[tert-butyl(dimethyl)silyl]oxyethyl]-2-ethylsulfanyl-3,6-dimethyl-chromen-4-one (0.720 g, 1.83 mmol) in DCM (12 mL) was cooled to 0° C. and treated in portions with mCPBA (0.452 g, 77%, 2.02 mmol). After addition was complete, the reaction was stirred at 0° C. for 1 h. The reaction was diluted with EtOAc and washed with saturated aqueous NaHCO₃. The organic layer was removed and the aqueous layer extracted with EtOAc (2×). The combined organic layers were dried over Na₂SO₄, filtered, and concentrated under reduced pressure. The residue was purified by silica gel chromatography eluted with 0% to 100% EtOAc in heptane to give the title compound (0.682 g, 91%) as a colorless gel. MS ES+ m/z 409 [M+H]⁺.

### Intermediate 25B: 8-[(1S)-1-[tert-Butyl(dimethyl)silyl]oxyethyl]-2-chloro-3,6-dimethyl-chromen-4-one

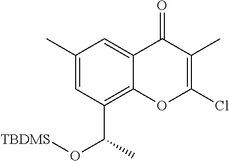

A 0° C. solution of 8-[(1S)-1-[tert-butyl(dimethyl)silyl]oxyethyl]-2-ethylsulfinyl-3,6-dimethyl-chromen-4-one (0.680 g, 1.66 mmol) and benzyl(triethyl) ammonium chloride (0.190 g, 0.832 mmol) in DCM (30 mL) was treated with aqueous HCl (12M, 0.417 mL, 4.99 mmol). After 1 h, additional aqueous HCl (12M, 0.417 mL, 4.99 mmol) was added. After an additional 1 h, additional aqueous HCl (12M, 0.417 mL, 4.99 mmol) was added. After 30 min, the reaction mixture was quenched with saturated aqueous NaHCO₃. The aqueous layer extracted with DCM. The organic layer was washed with saturated aqueous sodium chloride, dried over MgSO₄, filtered, and concentrated under reduced pressure. The residue was purified by silica gel chromatography eluted with 0% to 50% EtOAc in heptane to give the title compound (0.87 g, 77%). MS ES+ m/z 367 [M+H]⁺.

### Intermediate 26B: 8-[(1S)-1-[tert-Butyl(dimethyl)silyl]oxyethyl]-2-cyclopropyl-3,6-dimethyl-chromen-4-one

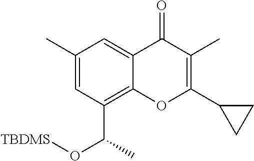

Cyclopropylzine iodide: An oven dried flask was charged with lithium chloride (2.52 g, 59.5 mmol) and heated under high vacuum at 170° C. for 20 min. The vacuum was removed and nitrogen gas introduced into the flask after cooling. Zinc (3.89 g, 59.5 mmol) was added to the flask when cool and dried under high vacuum at 170° C. for 20 min and backfilled with nitrogen after cooling. When cooled to rt, THE (20 mL) and 1,2-dibromoethane (0.13 mL g, 1.49 mmol) were added via syringe and the reaction heated at 60° C. until bubbling occurred. After cooling to rt, chlorotrimethylsilane (0.038 mL g, 0.30 mmol) and iodine (0.038 g, 0.15 mmol) in THE (1 mL) were added via syringe and the reaction heated at 60° C. for 20 min and then cooled to rt. Added iodocyclopropane (5.0 g, 29.8 mmol) and stirred the reaction at 50° C. overnight. The reaction was allowed to stand at rt for 1 h and the solution on top of the solids carefully transferred via cannula to a dry flask for use without purification assuming ˜0.5 M concentration.

A dry vial was charged with 8-[(1S)-1-[tert-butyl(dimethyl)silyl]oxyethyl]-2-chloro-3,6-dimethyl-chromen-4-one (0.167 g, 0.455 mmol), palladium(II) acetate (10.2 mg, 0.046 mmol), and 2-dicyclohexylphosphino-2′,6′-bis(N,N-dimethylamino)biphenyl (CPhos, 0.040 g, 0.091 mmol). The vial was evacuated and refilled with nitrogen three times. Added THE (3 mL) via syringe and cooled the reaction to 0° C. Added cyclopropylzinc iodide (˜0.5M, 2.28 mL, 1.14 mmol) via syringe, removed the cooling bath, and stirred overnight at rt. The reaction was diluted with EtOAc and washed with saturated aqueous NH₄Cl. The organic layer was removed and the aqueous layer re-extracted with EtOAc (2×). The organic layers were combined, dried over Na₂SO₄, filtered, and concentrated under reduced pressure. The residue was purified by silica gel chromatography eluted with 0% to 50% EtOAc in heptane to give the title compound (0.135 g, 80%) as a brown gel. MS ES+ m/z 373 [M+H]⁺.

### Intermediate 61C: ethyl 2-[8-[(1S)-1-[tert-Butyl(dimethyl)silyl]oxyethyl]-3,6-dimethyl-4-oxo-chromen-2-yl]cyclopropanecarboxylate

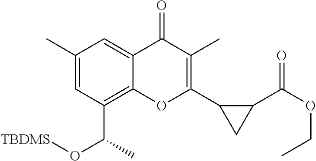

A microwave vial was charged with 8-[(1S)-1-[tert-butyl(dimethyl)silyl]oxyethyl]-2-chloro-3,6-dimethyl-chromen-4-one (0.500 g, 1.36 mmol), bis(1-adamantyl)-butyl-phosphane (0.147 g, 0.409 mmol), (2-(ethoxycarbonyl)cyclopropyl)trifluoroborate, potassium salt (0.5M; 5.45 mL, 2.73 mmol), cesium carbonate (1.33 g, 4.09 mmol), and palladium diacetate (0.061 g, 0.273 mmol). The vial was sealed and cycled between vacuum and nitrogen three times. Toluene (10 mL) and water (1 mL) were added via syringe. The vial was heated in a microwave reactor at 110° C. for 1 h, then diluted with EtOAc. The mixture was washed with water and the aqueous layer was extracted with EtOAc (2×). The combined organic layers were dried over sodium sulfate, filtered, and concentrated under reduced pressure. The residue was purified by silica gel chromatography eluted with 0% to 100% EtOAc in heptane to give the title compound (0.709 g, 99%) as a tan gel. MS ES+ m/z 445 [M+H]⁺.

The following compounds in Table 11 were made in a similar way as described for ethyl 2-[8-[(1S)-1-[tert-Butyl(dimethyl)silyl]oxyethyl]-3,6-dimethyl-4-oxo-chromen-2-yl]cyclopropanecarboxylate. Various methods were used to purify the compounds, which would be apparent to one skilled in the art.

TABLE 11

| | | | MS ES+ |
|---|---|---|---|
| Interm # | Chemical Name | Structure | m/z |
| 62C | 8-[(1S)-1-[tert- Butyl(dimethyl)silyl]oxyethyl]- 3,6-dimethyl-2-(5- oxaspiro[2.3]hexan-2- yl)chromen-4-one | 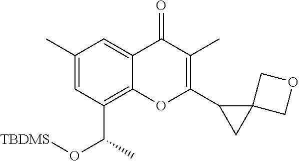 | 415 [M + H]⁺ |
| 63C | ethyl 2-[1-[8-[(1S)-1-[tert- Butyl(dimethyl)silyl]oxyethyl]- 3,6-dimethyl-4-oxo-chromen- 2-yl]cyclopropyl]acetate | 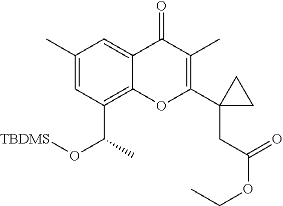 | 459 [M + H]⁺ |

### Intermediate 64C: 8-[(1S)-1-[tert-Butyl(dimethyl)silyl]oxyethyl]-3,6-dimethyl-2-(oxetan-3-yl)chromen-4-one

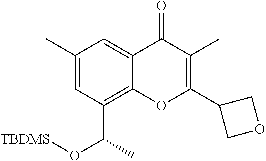

To a small vial was added photocatalyst (Ir[dF(CF₃)ppy]₂(dtbpy))PF₆ (0.0076 g, 0.0068 mmol), 3-bromooxetane (0.140 g, 1.02 mmol), 8-[(1S)-1-[tert-butyl(dimethyl)silyl]oxyethyl]-2-chloro-3,6-dimethyl-chromen-4-one (0.250 g, 0.681 mmol), tris(trimethylsilyl)silane (0.21 mL, 0.681 mmol), and lutidine (0.16 mL, 1.36 mmol). The vial was sealed and placed under nitrogen, then dimethoxyethane (9 mL) was added. To a second vial was added (4,4′-di-tert-butyl-2,2′-bipyridine)NiCl₂ (0.0014 g, 0.0034 mmol) and dimethoxyethane (1 mL). The mixture was degassed and sonicated for 3 min, then added via syringe to the first vial. The vial was cycled between vacuum and nitrogen three times, then placed in a photobox equipped with a fan and blue LED lamp. The reaction mixture was stirred overnight, then filtered through diatomaceous earth. The filtrate was concentrated under reduced pressure to give the crude title compound. MS ES+ m/z 389 [M+H]⁺.

### Intermediate 27B: 2-Cyclopropyl-8-[(1S)-1-hydroxyethyl]-3,6-dimethyl-chromen-4-one

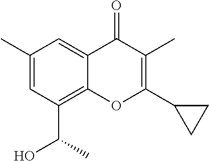 [SMILES: Cc1cc([C@H](C)O)c2oc(C3CC3)c(C)c(=O)c2c1]

A 0° C. solution of 8-[(1S)-1-[tert-butyl(dimethyl)silyl]oxyethyl]-2-cyclopropyl-3,6-dimethyl-chromen-4-one (0.135 g, 0.362 mmol) in THE (3 mL) was treated with tetra-n-butylammonium fluoride (1M in THF, 0.435 mL, 0.435 mmol). The reaction was stirred at 0° C. for 2.5 h, then concentrated under reduced pressure. The residue was purified by reversed phase chromatography eluted with a gradient of 0 to 100% ACN in H₂O+0.1% formic acid. Fractions containing the desired product were combined, washed with saturated aqueous sodium chloride, and extracted with IPA:CHCl₃ (1:3). The combined extracts were dried over MgSO₄, filtered, and concentrated under reduced pressure to give the crude title compound. MS ES+ m/z 259 [M+H]⁺.

The following compounds in Table 12 were made in a similar way as described for 2-cyclopropyl-8-[(1S)-1-hydroxyethyl]-3,6-dimethyl-chromen-4-one. Various methods were used to purify the compounds, which would be apparent to one skilled in the art.

TABLE 12

| | | | MS ES+ |
|---|---|---|---|
| Interm # | Chemical Name | Structure | m/z |
| 65C | ethyl 2-[8-[(1S)-1- Hydroxyethyl]-3,6- dimethyl-4-oxo-chromen- 2- yl]cyclopropanecarboxylate | 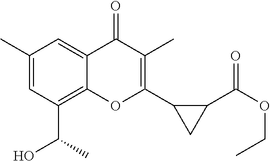 [SMILES: CCOC(=O)C1CC1c1oc2c([C@H](C)O)cc(C)cc2c(=O)c1C] | 331 [M + H]⁺ |
| 66C | 8-[(1S)-1-Hydroxyethyl]- 3,6-dimethyl-2-(5- oxaspiro[2.3]hcxan-2- yl)chromen-4-one | 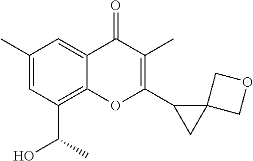 [SMILES: Cc1cc([C@H](C)O)c2oc(C3CC34COC4)c(C)c(=O)c2c1] | 301 [M + H]⁺ |
| 67C | ethyl 2-[1-[8-[(1S)-1- Hydroxyethyl]-3,6- dimethyl-4-oxo-chromen- 2-yl]cyclopropyl]acetate | 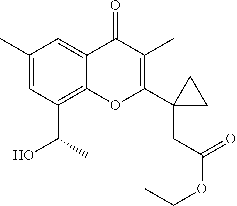 [SMILES: CCOC(=O)CC1(c2oc3c([C@H](C)O)cc(C)cc3c(=O)c2C)CC1] | 345 [M + H]⁺ |
| 68C | 8-[(1S)-1-Hydroxyethyl]- 3,6-dimethyl-2-(oxetan-3- yl)chromen-4-one | 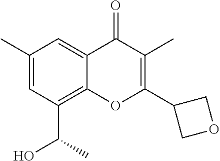 [SMILES: Cc1cc([C@H](C)O)c2oc(C3COC3)c(C)c(=O)c2c1] | 275 [M + H]⁺ |

### Intermediate 9A: 8-[(1S)-1-Hydroxyethyl]3,6-dimethyl-2-(2-pyridyl)chromen-4-one

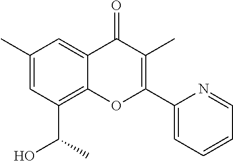 [SMILES: Cc1cc([C@H](C)O)c2oc(-c3ccccn3)c(C)c(=O)c2c1]

Combined 2-ethylsulfanyl-8-[(1S)-1-hydroxyethyl]-3,6-dimethyl-chromen-4-one (500 mg, 1.80 mmol), 2-pyridylboronic acid (662 mg, 5.39 mmol), copper(I) thiophene-2-carboxylate (514 mg, 2.69 mmol) and tetrakis(triphenylphosphine)palladium(0) (415 mg, 0.36 mmol) in EtOH (10 mL), degassed with nitrogen for 2 min, and stirred at 60° C. for 16 h. The reaction was recharged with 2-pyridylboronic acid (662 mg, 5.39 mmol), copper(I) thiophene-2-carboxylate (514 mg, 2.69 mmol) and tetrakis(triphenylphosphine)palladium(0) (415 mg, 0.36 mmol), purged with nitrogen for 5 min, and stirred at 60° C. for 4 h. The reaction was filtered and the solids washed with DCM/MeOH. The filtrate was concentrated under reduced pressure and the residue purified by reversed phase chromatography on C18 eluted with 0% to 100% ACN in 10 mM aqueous NH₄HCO₃ with 5% MeOH to give the title compound (281 mg, 53%) as a tan solid. ES/MS m/z 296.2 (M+H).

The following compounds in Table 13 were made in a similar way as described for 8-[(1S)-1-hydroxyethyl]-3,6-dimethyl-2-(2-pyridyl)chromen-4-one. Various methods were used to purify the compounds, which would be apparent to one skilled in the art.

TABLE 13

| Interm | | | MS ES+ |
|---|---|---|---|
| # | Chemical Name | Structure | m/z |
| 10A | 8-[(1S)-1- Hydroxyethyl]-3,6- dimethyl-2-(1- methylpyrazol-4- yl)chromen-4-one | 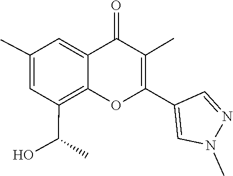 [SMILES: Cc1cc([C@H](C)O)c2oc(-c3cnn(C)c3)c(C)c(=O)c2c1] | 299 [M + H]⁺ |
| 11A | 8-[(1S)-1- Hydroxyethyl]-3,6- dimethyl-2-(3-pyridyl) chromen-4-one | 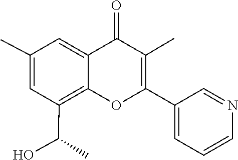 [SMILES: Cc1cc([C@H](C)O)c2oc(-c3cccnc3)c(C)c(=O)c2c1] | 296 [M + H]⁺ |
| 12A | 2-(2-Fluoro-3- pyridyl)-8-[(1S)-1- hydroxyethyl]- 3,6-dimethyl- chromen-4-one | 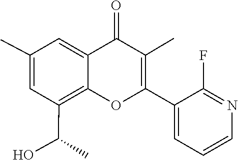 [SMILES: Cc1cc([C@H](C)O)c2oc(-c3cccnc3F)c(C)c(=O)c2c1] | 314 [M + H]⁺ |
| 13A | 2-(5-Fluoro-3- pyridyl)-8-[(1S)-1- hydroxyethyl]- 3,6-dimethyl- chromen-4-one | 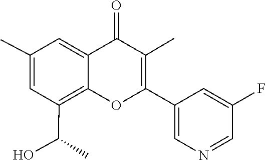 [SMILES: Cc1cc([C@H](C)O)c2oc(-c3cncc(F)c3)c(C)c(=O)c2c1] | 314 [M + H]⁺ |
| 14A | 2-(2,5-Difluoro- 3-pyridyl)-8- [(1S)-1-hydroxyethyl]- 3,6- dimethyl- chromen-4-one | 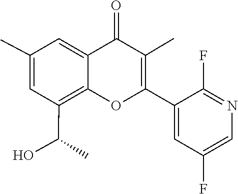 [SMILES: Cc1cc([C@H](C)O)c2oc(-c3cc(F)cnc3F)c(C)c(=O)c2c1] | 332 [M + H]⁺ |
| 15A | 6-Chloro-3-[(1R)-1- [2-(3-fluoro- 2-pyridyl)-3,6- dimethyl-4-oxo- chromen-8-yl]ethoxy] pyridine-2- carbonitrile | 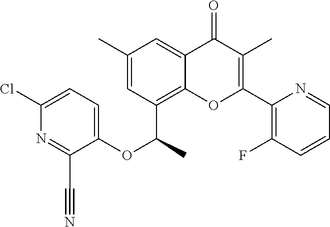 [SMILES: Cc1cc([C@@H](C)Oc2ccc(Cl)nc2C#N)c2oc(-c3ncccc3F)c(C)c(=O)c2c1] | 450 [M + H]⁺ |
| 16A | 6-Chloro-3-[(1R)- 1-[2-(6-fluoro- 2-pyridyl)-3,6- dimethyl-4-oxo- chromen-8-yl] ethoxy]pyridine-2- carbonitrile | 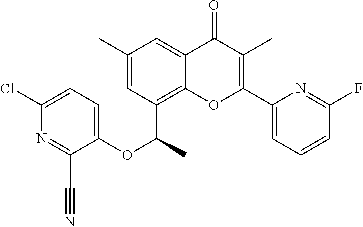 [SMILES: Cc1cc([C@@H](C)Oc2ccc(Cl)nc2C#N)c2oc(-c3cccc(F)n3)c(C)c(=O)c2c1] | 450 [M + H]⁺ |
| 17A | 8-[(1S)-1- Hydroxyethyl]-3,6- dimethyl-2-(2- methylindazol-5- yl)chromen-4-one | 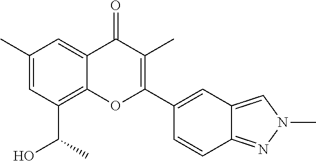 | 349 [M + H]⁺ |
| 18A | 8-[(1R)-1-[(6-Chloro- 2-fluoro-3- pyridyl)oxy]ethyl]- 3,6-dimethyl- 2-(1-methylpyrazol- 4-yl)chromen- 4-one | 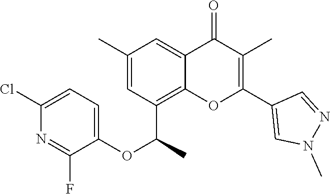 [SMILES: Cc1cc([C@@H](C)Oc2ccc(Cl)nc2F)c2oc(-c3cnn(C)c3)c(C)c(=O)c2c1] | 428 [M + H]⁺ |
| 19A | Methyl 6-[(1R)-1-[3,6- dimethyl-2- (2-methylindazol- 5-yl)-4-oxo- chromen-8-yl] ethoxy]-2,3- difluoro-benzoate | 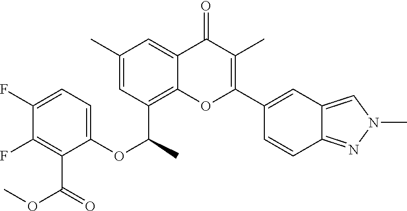 [SMILES: COC(=O)c1c(O[C@H](C)c2cc(C)cc3c(=O)c(C)c(-c4ccc5nn(C)cc5c4)oc23)ccc(F)c1F] | 519 [M + H]⁺ |
| 20A | 8-[(1R)-1-[(6-Chloro- 2-fluoro-3- pyridyl)oxy]ethyl]-2- ([1,3-dioxolo[4,5-b] pyridin-6-yl)- 3,6-dimethyl- chromen-4-one | 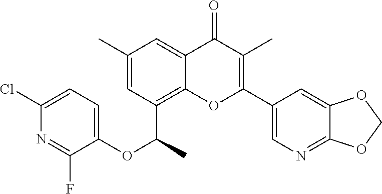 [SMILES: Cc1cc([C@@H](C)Oc2ccc(Cl)nc2F)c2oc(-c3cnc4c(c3)OCO4)c(C)c(=O)c2c1] | 469 [M + H]⁺ |
| 28B | 8-[(1S)-1- Hydroxyethyl]-3,6- dimethyl-2-(2- methylthiazol-4- yl)chromen-4-one | 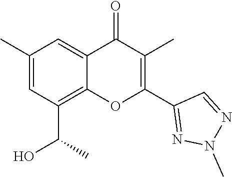 [SMILES: Cc1cc([C@H](C)O)c2oc(-c3cnn(C)n3)c(C)c(=O)c2c1] | 300 [M + H]⁺ |
| 29B | 6-Chloro-3- [(1R)-1-[2- ([1,3]dioxolo[4,5-b] pyridin-6-yl)- 3,6-dimethyl-4- oxo-chromen-8- yl]ethoxy]pyridine- 2-carbonitrile | 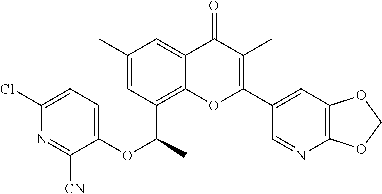 [SMILES: Cc1cc([C@@H](C)Oc2ccc(Cl)nc2C#N)c2oc(-c3cnc4c(c3)OCO4)c(C)c(=O)c2c1] | 476 [M + H]⁺ |
| 30B | 2-(6,7-Dihydro- 4H-pyrazolo[5,1- c][1,4]oxazin-3-yl)- 8-[(1S)-1- hydroxyethyl]- 3,6-dimethyl- chromen-4-one | 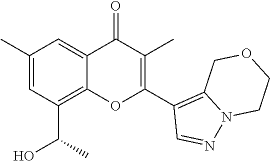 [SMILES: Cc1cc([C@H](C)O)c2oc(-c3cnn4c3COCC4)c(C)c(=O)c2c1] | 341 [M + H]⁺ |
| 31B | 6-Chloro-3- [(1R)-1-[2-(2,2- difluoro-1,3- benzodioxol-5-yl)- 3,6-dimethyl-4- oxo-chromen-8- yl]ethoxy]pyridine- 2-carbonitrile | 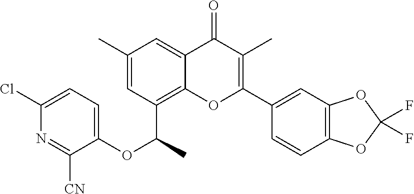 [SMILES: Cc1cc([C@@H](C)Oc2ccc(Cl)nc2C#N)c2oc(-c3ccc4c(c3)OC(F)(F)O4)c(C)c(=O)c2c1] | 511 [M + H]⁺ |
| 32B | 6-Chloro-3- [(1R)-1-[2-(2,3- dihydro-[1,4]dioxino[2,3- b]pyridin-7-yl)- 3,6-dimethyl-4- oxo-chromen-8- yl]ethoxy]pyridine- 2-carbonitrile | 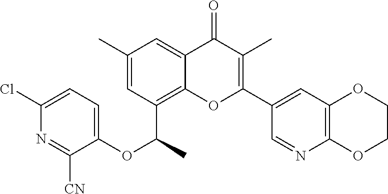 [SMILES: Cc1cc([C@@H](C)Oc2ccc(Cl)nc2C#N)c2oc(-c3cnc4c(c3)OCCO4)c(C)c(=O)c2c1] | 490 [M + H]⁺ |
| 33B | 6-Chloro-3-[(1R)- 1-[3,6-dimethyl- 2-(4-methyl- 2,3-dihydro-1,4- benzoxazin-7-yl)- 4-oxo-chromen- 8-yl]ethoxy]pyridine-2- carbonitrile | 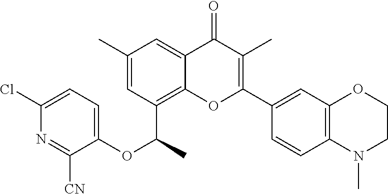 [SMILES: Cc1cc([C@@H](C)Oc2ccc(Cl)nc2C#N)c2oc(-c3ccc4c(c3)OCCN4C)c(C)c(=O)c2c1] | 502 [M + H]⁺ |
| 34B | 6-Chloro-3-[(1R)- 1-[3,6-dimethyl- 2-(4-methyl-2,3- dihydropyrido[3,2-b] [1,4]oxazin- 7-yl)-4-oxo- chromen-8- yl]ethoxy]pyridine- 2-carbonitrile | 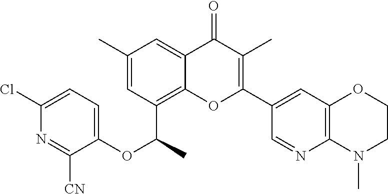 [SMILES: Cc1cc([C@@H](C)Oc2ccc(Cl)nc2C#N)c2oc(-c3cnc4c(c3)OCCN4C)c(C)c(=O)c2c1] | 503 [M + H]⁺ |
| 35B | 6-Chloro-3-[(1R)- 1-[3,6-dimethyl- 2-(1,5-naphthyridin- 3-yl)-4-oxo- chromen-8-yl]ethoxy] pyridine-2- carbonitrile | 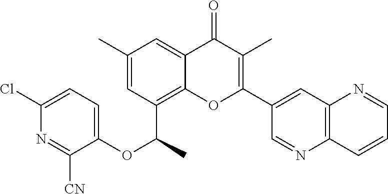 [SMILES: Cc1cc([C@@H](C)Oc2ccc(Cl)nc2C#N)c2oc(-c3cnc4cccnc4c3)c(C)c(=O)c2c1] | 483 [M + H]⁺ |
| 36B | 6-Chloro-3-[(1R)- 1-[2-(5,6- dihydro-4H-pyrrolo [2,1-e]pyrazol- 3-yl)-3,6-dimethyl-4-oxo- chromen-8-yl] ethoxy]pyridine-2- carbonitrile | 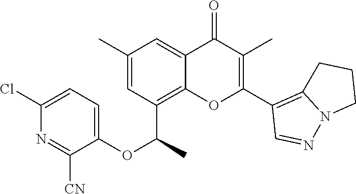 [SMILES: Cc1cc([C@@H](C)Oc2ccc(Cl)nc2C#N)c2oc(-c3cnn4c3CCC4)c(C)c(=O)c2c1] | 461 [M + H]⁺ |
| 37B | 3-[(1R)-1-[3,6- Dimethyl-2-(2- methylindazol-5-yl)-4-oxo- chromen-8-yl] ethoxy]-6-fluoro- pyridine-2-carbonitrile | 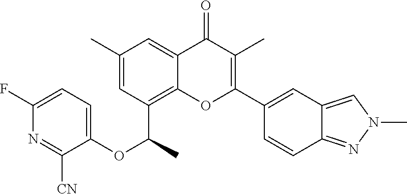 [SMILES: Cc1cc([C@@H](C)Oc2ccc(F)nc2C#N)c2oc(-c3ccc4nn(C)cc4c3)c(C)c(=O)c2c1] | 469 [M + H]⁺ |
| 38B | 6-Chloro-3-[(1R)-1- (3,6-dimethyl- 4-oxo-2-thiazolo [5,4-b]pyridin-6- yl-chromen-8-yl) ethoxy]pyridine- 2-carbonitrile | 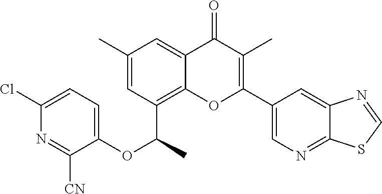 [SMILES: Cc1cc([C@@H](C)Oc2ccc(Cl)nc2C#N)c2oc(-c3cnc4scnc4c3)c(C)c(=O)c2c1] | 489 [M + H]⁺ |
| 39B | 8-[(1R)-1-[(6- Chloro-2-fluoro-3- pyridyl)oxy]ethyl]- 3,6-dimethyl- 2-(2-methyltriazol- 4-yl)chromen-4-one | 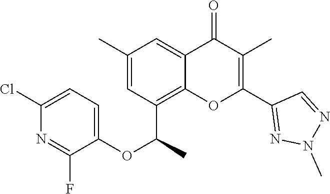 [SMILES: Cc1cc([C@@H](C)Oc2ccc(Cl)nc2F)c2oc(-c3cnn(C)n3)c(C)c(=O)c2c1] | 429 [M + H]⁺ |
| 40B | 8-[(1R)-1-[(6-Chloro- 2-fluoro-3- pyridyl)oxy]ethyl]- 3,6-dimethyl- 2-pyrimidin-5-yl- chromen-4-one | 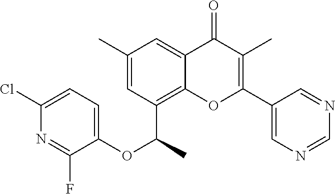 [SMILES: Cc1cc([C@@H](C)Oc2ccc(Cl)nc2F)c2oc(-c3cncnc3)c(C)c(=O)c2c1] | 426 [M + H]⁺ |
| 41B | 8-[(1R)-1-[(6- Chloro-2-fluoro-3- pyridyl)oxy]ethyl]- 3,6-dimethyl- 2-(2-methylpyrimidin-5- yl)chromen-4-one | 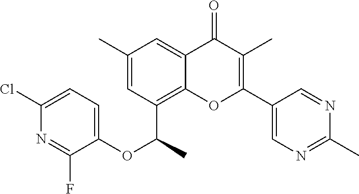 [SMILES: Cc1cc([C@@H](C)Oc2ccc(Cl)nc2F)c2oc(-c3cnc(C)nc3)c(C)c(=O)c2c1] | 440 [M + H]⁺ |
| 42B | 8-[(1R)-1-[(6- Chloro-2-fluoro-3- pyridyl)oxy]ethyl]-2-(2- cyclopropylpyrimidin- 5-yl)-3,6- dimethyl- chromen-4-one | 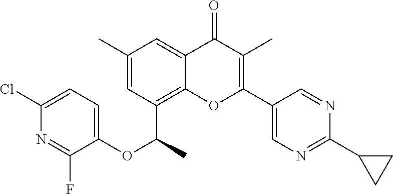 [SMILES: Cc1cc([C@@H](C)Oc2ccc(Cl)nc2F)c2oc(-c3cnc(C4CC4)nc3)c(C)c(=O)c2c1] | 466 [M + H]⁺ |
| 43B | 8-[(1R)-1-[(6- Chloro-2-fluoro-3- pyridyl)oxy]ethyl]-2-[1-(2- hydroxy-2-methyl- propyl)pyrazol- 4-yl]-3,6-dimethyl- chromen-4-one | 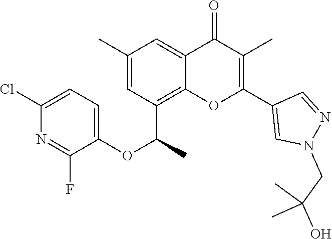 [SMILES: Cc1cc([C@@H](C)Oc2ccc(Cl)nc2F)c2oc(-c3cnn(CC(C)(C)O)c3)c(C)c(=O)c2c1] | 486 [M + H]⁺ |
| 44B | 8-[(1R)-1-[(6- Chloro-2-fluoro-3- pyridyl)oxy]ethyl]- 2-(6,7-dihydro- 4H-pyrazolo[5,1-c] [1,4]oxazin-3- yl)-3,6-dimethyl- chromen-4-one | 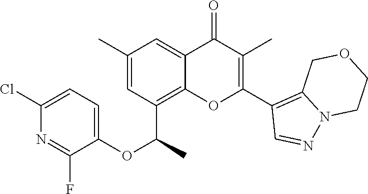 [SMILES: Cc1cc([C@@H](C)Oc2ccc(Cl)nc2F)c2oc(-c3cnn4c3COCC4)c(C)c(=O)c2c1] | 470 [M + H]⁺ |
| 45B | 8-[(1R)-1-[(6- Chloro-2-fluoro-3- pyridyl)oxy]ethyl]-2- [1-[(2R)-2- hydroxypropyl] pyrazol-4-yl]-3,6- dimethyl-chromen-4-one | 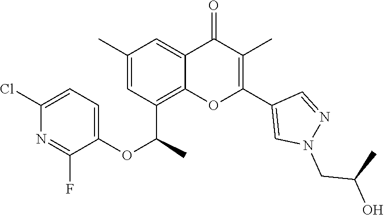 [SMILES: Cc1cc([C@@H](C)Oc2ccc(Cl)nc2F)c2oc(-c3cnn(C[C@@H](C)O)c3)c(C)c(=O)c2c1] | 472 [M + H]⁺ |
| 46B | 8-[(1R)-1-[(6- Chloro-2-fluoro-3- pyridyl)oxy]ethyl]- 3,6-dimethyl- 2-(2-methylpyrazolo[4,3- b]pyridin-5-yl) chromen-4-one | 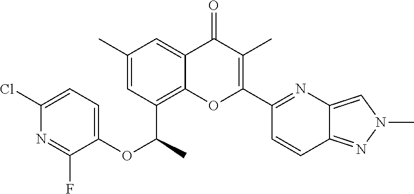 [SMILES: Cc1cc([C@@H](C)Oc2ccc(Cl)nc2F)c2oc(-c3ccc4nn(C)cc4n3)c(C)c(=O)c2c1] | 479 [M + H]⁺ |
| 47B | 8-[(1R)-1-[(6- Chloro-2-fluoro-3- pyridyl)oxy]ethyl]- 2-(2,3-dihydro- [1,4]dioxino[2,3-b] pyridin-7-yl)- 3,6-dimethyl- chromen-4-one | 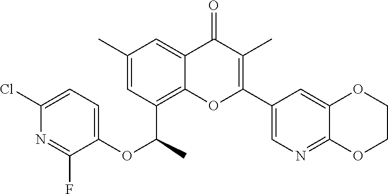 [SMILES: Cc1cc([C@@H](C)Oc2ccc(Cl)nc2F)c2oc(-c3cnc4c(c3)OCCO4)c(C)c(=O)c2c1] | 483 [M + H]⁺ |
| 48B | 8-[(1R)-1-[(6- Chloro-2-fluoro-3- pyridyl)oxy]ethyl]- 2-[1-[(2S)-2- hydroxypropyl]pyrazol- 4-yl]-3,6- dimethyl-chromen-4-one | 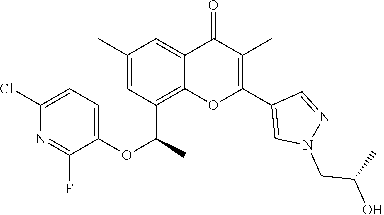 [SMILES: Cc1cc([C@@H](C)Oc2ccc(Cl)nc2F)c2oc(-c3cnn(C[C@H](C)O)c3)c(C)c(=O)c2c1] | 472 [M + H]⁺ |
| 49B | 8-[(1S)-1- Hydroxyethyl]-3,6- dimethyl-2-(2- methylindazol-5- yl)chromen-4-one | 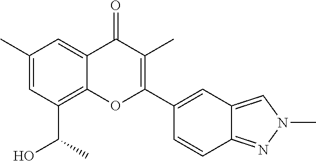 | 349 [M + H]⁺ |
| 50B | methyl 6-Chloro- 3-[(1R)-1-[3,6- dimethyl-2-(1- methylpyrazol-4- yl)-4-oxo-chromen-8- yl]ethoxy]pyridine- 2-carboxylate | 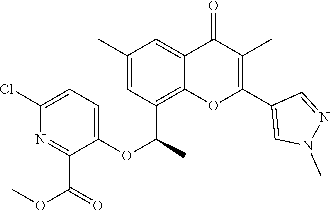 [SMILES: COC(=O)c1nc(Cl)ccc1O[C@H](C)c1cc(C)cc2c(=O)c(C)c(-c3cnn(C)c3)oc12] | 468 [M + H]⁺ |
| 51B | 2-[1-[2-[tert- Butyl(dimethyl) silyl]oxyethyl] pyrazol-4-yl]-8-[(1S)-1- hydroxyethyl]-3,6-dimethyl- chromen-4-one | 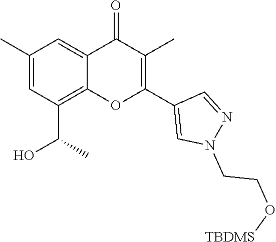 | 443 [M + H]⁺ |
| 52B | 8-Acetyl-6- methyl-2-(1- methylpyrazol-4- yl)chromen-4-one | 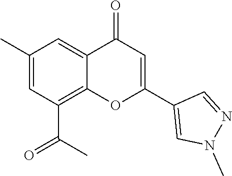 [SMILES: CC(=O)c1cc(C)cc2c(=O)cc(-c3cnn(C)c3)oc12] | 283 [M + H]⁺ |
| 53B | 6-Chloro-3-[(1R)- 1-[6-methyl-2- (2-methylindazol- 5-yl)-4-oxo- chromen-8-yl]ethoxy] pyridine-2- carbonitrile | 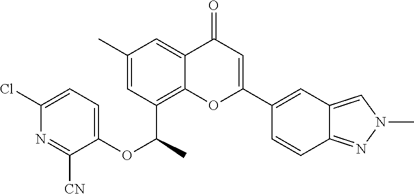 [SMILES: Cc1cc([C@@H](C)Oc2ccc(Cl)nc2C#N)c2oc(-c3ccc4nn(C)cc4c3)cc(=O)c2c1] | 471 [M + H]⁺ |
| 54B | 6-Chloro-3-[(1R)- 1-(6-methyl-4- oxo-2-phenyl- chromen-8- yl)ethoxy]pyridine- 2-carbonitrile | 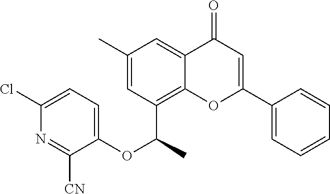 | 471 [M + H]⁺ |
| 55B | 6-Chloro-3-[(1R)-1-[2-(1H- indazol-5-yl)-3,6- dimethyl-4-oxo- chromen-8-yl]ethoxy] pyridine-2- carbonitrile | 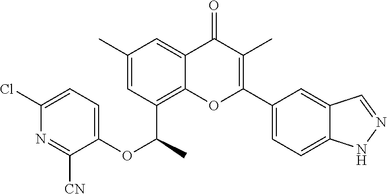 [SMILES: Cc1cc([C@@H](C)Oc2ccc(Cl)nc2C#N)c2oc(-c3ccc4[nH]ncc4c3)c(C)c(=O)c2c1] | 471 [M + H]⁺ |
| 56B | 3-[(1R)-1-[3,6- Dimethyl-2-(1- methylpyrazol- 4-yl)-4-oxo- chromen-8-yl] ethoxy]-6-fluoro- pyridine-2-carbonitrile | 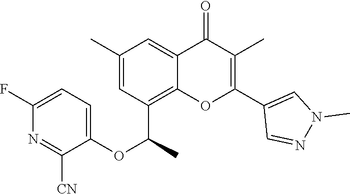 [SMILES: Cc1cc([C@@H](C)Oc2ccc(F)nc2C#N)c2oc(-c3cnn(C)c3)c(C)c(=O)c2c1] | 417 [M − H]⁻ |
| 69C | 6-Chloro-3-[(1R)-1- [3,6-dimethyl- 2-(2-methylpyrazolo[4,3- b]pyridin-5-yl)-4- oxo-chromen-8- yl]ethoxy]pyridine- 2-carbonitrile | 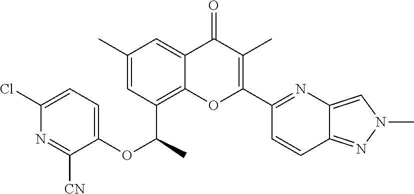 [SMILES: Cc1cc([C@@H](C)Oc2ccc(Cl)nc2C#N)c2oc(-c3ccc4nn(C)cc4n3)c(C)c(=O)c2c1] | 486 [M + H]⁺ |
| 70C | 6-Chloro-3-[(1R)- 1-[3,6-dimethyl- 2-(1-methylpyrazolo[3,4- b]pyridin-6-yl)-4- oxo-chromen-8- yl]ethoxy]pyridine- 2-carbonitrile | 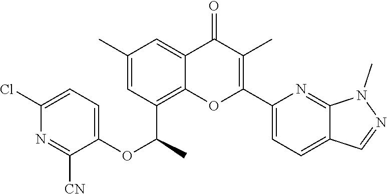 [SMILES: Cc1cc([C@@H](C)Oc2ccc(Cl)nc2C#N)c2oc(-c3ccc4cnn(C)c4n3)c(C)c(=O)c2c1] | 486 [M + H]⁺ |
| 71C | 6-Chloro-3-[(1R)-1- (3,6-dimethyl- 4-oxo-2-pyrazin-2- yl-chromen-8- yl)ethoxy]pyridine- 2-carbonitrile | 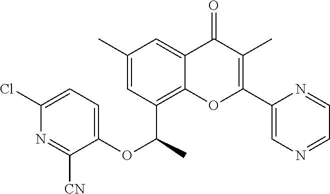 [SMILES: Cc1cc([C@@H](C)Oc2ccc(Cl)nc2C#N)c2oc(-c3cnccn3)c(C)c(=O)c2c1] | 433 [M + H]⁺ |
| 72C | 6-Chloro-3-[(1R)- 1-[3,6-dimethyl- 2-(2-methylimidazo [1,2-a]pyridin- 6-yl)-4-oxo-chromen-8- yl]ethoxy]pyridine- 2-carbonitrile | 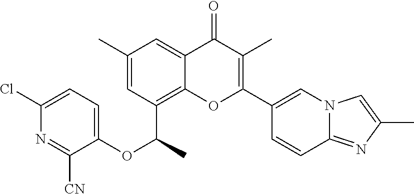 [SMILES: Cc1cc([C@@H](C)Oc2ccc(Cl)nc2C#N)c2oc(-c3ccc4nc(C)cn4c3)c(C)c(=O)c2c1] | 485 [M + H]⁺ |
| 73C | 6-Chloro-3- [(1R)-1-[2-(2- isopropoxypyrimidin- 5-yl)-3,6- dimethyl-4-oxo- chromen-8- yl]ethoxy]pyridine- 2-carbonitrile | 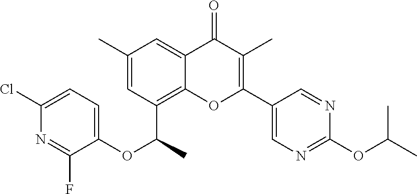 | 491 [M + H]⁺ |
| 74C | 2-Furo[3,2-b]pyridin- 6-yl-8-[(1S)- 1-hydroxyethyl]- 3,6-dimethyl- chromen-4-one | 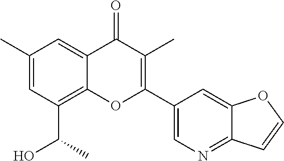 [SMILES: Cc1cc([C@H](C)O)c2oc(-c3cnc4ccoc4c3)c(C)c(=O)c2c1] | 336 [M + H]⁺ |
| 75C | 2-(2,3- Dihydropyrazolo[5,1- b]oxazol-7-yl)-8-[(1S)-1- hydroxyethyl]-3,6-dimethyl- chromen-4-one | 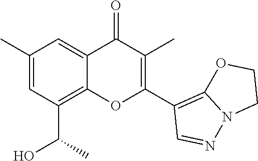 [SMILES: Cc1cc([C@H](C)O)c2oc(-c3cnn4c3OCC4)c(C)c(=O)c2c1] | 327 [M + H]⁺ |
| 76C | 2-(6,8-Dihydro- 5H-imidazo[2,1- c][1,4]oxazin-3- yl)-8-[(1S)-1- hydroxyethyl]- 3,6-dimethyl- chromen-4-one | 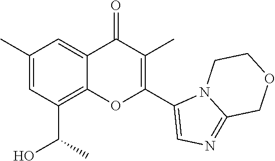 [SMILES: Cc1cc([C@H](C)O)c2oc(-c3cnc4n3CCOC4)c(C)c(=O)c2c1] | 341 [M + H]⁺ |
| 77C | 8-[(1S)-1-Hydroxyethyl]- 3,6- dimethyl-2-(1,5- naphthyridin-3- yl)chromen-4-one | 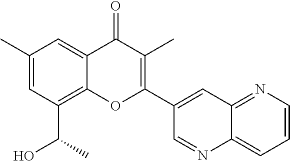 [SMILES: Cc1cc([C@H](C)O)c2oc(-c3cnc4cccnc4c3)c(C)c(=O)c2c1] | 347 [M + H]⁺ |
| 78C | 2-[2-[2-[tert- Butyl(dimethyl)silyl] oxyethyl]indazol- 5-yl]-8-[(1S)-1- hydroxyethyl]- 3,6-dimethyl- chromen-4-one | 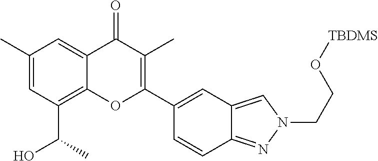 | 493 [M + H]⁺ |
| 79C | 2-(6,8-Dihydro- 5H-imidazo[2,1- c][1,4]oxazin-2-yl)-8- [(1S)-1- hydroxyethyl]- 3,6-dimethyl- chromen-4-one | 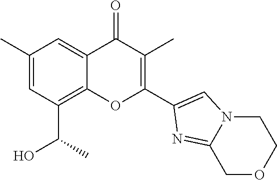 [SMILES: Cc1cc([C@H](C)O)c2oc(-c3cn4c(n3)COCC4)c(C)c(=O)c2c1] | 341 [M + H]⁺ |
| 80C | 8-[(1S)-1- Hydroxyethyl]-3,6- dimethyl-2-[1-(2- methylsulfonylethyl) pyrazol-4- yl]chromen-4-one | 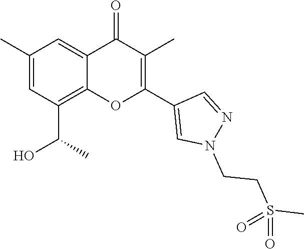 | 391 [M + H]⁺ |
| 81C | 8-[(1S)-1- Hydroxyethyl]-3,6- dimethyl-2-(1- methylimidazol-4- yl)chromen-4-one | 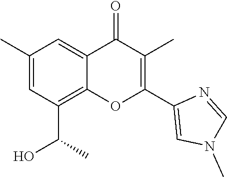 [SMILES: Cc1cc([C@H](C)O)c2oc(-c3cn(C)cn3)c(C)c(=O)c2c1] | 299 [M + H]⁺ |
| 82C | 8-[(1R)-1- Hydroxyethyl]-3,6- dimethyl-2-(1- tetrahydropyran-2- ylpyrazol-4-yl) chromen-4-one | 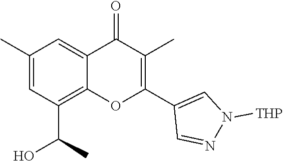 | 369 [M + H]⁺ |
| 83C | 8-[(1R)-1- Hydroxyethyl]-3,6- dimethyl-2-[1-(2- oxabicyclo [2.1.1]hexan-4- ylmethyl)pyrazol- 4-yl]chromen-4- one | 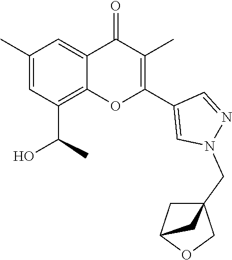 [SMILES: Cc1cc([C@@H](C)O)c2oc(-c3cnn(C[C@]45CO[C@H](C4)C5)c3)c(C)c(=O)c2c1] | 381 [M + H]⁺ |
| 84C | 8-[(1R)-1- Hydroxyethyl]-2-[1-(2- methoxyethyl)pyrazol- 4-yl]-3,6- dimethyl-chromen-4-one | 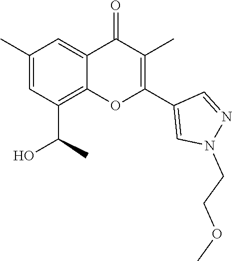 [SMILES: COCCn1cc(-c2oc3c([C@@H](C)O)cc(C)cc3c(=O)c2C)cn1] | 343 [M + H]⁺ |
| 85C | 8-[(1R)-1- Hydroxyethyl]-3,6- dimethyl-2-[1-(2- oxabicyclo[2.1.1]hexan-1- ylmethyl)pyrazol- 4-yl]chromen-4- one | 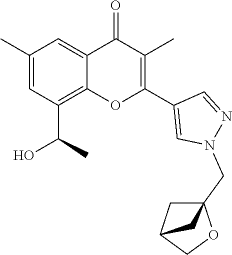 [SMILES: Cc1cc([C@@H](C)O)c2oc(-c3cnn(C[C@]45C[C@H](CO4)C5)c3)c(C)c(=O)c2c1] | 381 [M + H]⁺ |
| 86C | 2-[1-(3,3- Difluoropropyl)pyrazol- 4-yl]-8-[(1R)-1- hydroxyethyl]- 3,6-dimethyl- chromen-4-one | 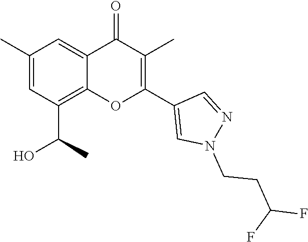 [SMILES: Cc1cc([C@@H](C)O)c2oc(-c3cnn(CCC(F)F)c3)c(C)c(=O)c2c1] | 363 [M + H]⁺ |
| 87C | 8-[(1R)-1- Hydroxyethyl]-3,6- dimethyl-2-(2- methylimidazo[1,2- a]pyridin-6-yl) chromen-4-one | 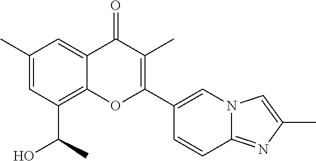 | 349 [M + H]⁺ |
| 88C | 8-[(1R)-1- Hydroxyethyl]-2-[1-(2- hydroxy-2-methyl- propyl)pyrazol- 4-yl]-3,6-dimethyl- chromen-4-one | 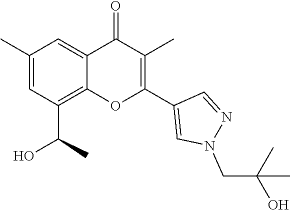 [SMILES: Cc1cc([C@@H](C)O)c2oc(-c3cnn(CC(C)(C)O)c3)c(C)c(=O)c2c1] | 357 [M + H]⁺ |
| 89C | 6-Chloro-3- [(1R)-1-[2-(2- ethylindazol-5-yl)- 3,6-dimethyl-4- oxo-chromen-8- yl]ethoxy]pyridine- 2-carbonitrile | 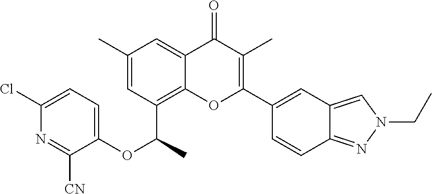 [SMILES: CCn1cc2cc(-c3oc4c([C@@H](C)Oc5ccc(Cl)nc5C#N)cc(C)cc4c(=O)c3C)ccc2n1] | 499 [M + H]⁺ |
| 90C | 6-Chloro-3-[(1R)- 1-[3,6-dimethyl- 2-(2-methylpyrazolo[1,5- a]pyridin-5-yl)-4- oxo-chromen-8- yl]ethoxy]pyridine- 2-carbonitrile | 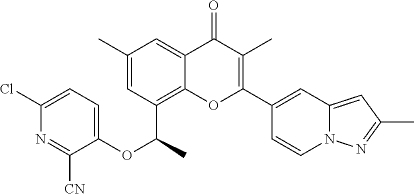 [SMILES: Cc1cc([C@@H](C)Oc2ccc(Cl)nc2C#N)c2oc(-c3ccn4nc(C)cc4c3)c(C)c(=O)c2c1] | 485 [M + H]⁺ |
| 91C | 6-Chloro-3-[(1R)- 1-[3,6-dimethyl- 2-[2-(oxetan-3- yl)indazol-5-yl]-4- oxo-chromen-8- yl]ethoxy]pyridine- 2-carbonitrile | 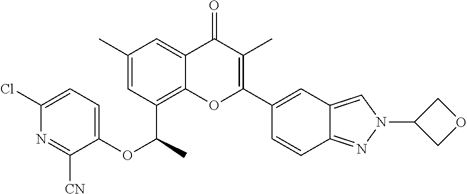 [SMILES: Cc1cc([C@@H](C)Oc2ccc(Cl)nc2C#N)c2oc(-c3ccc4nn(C5COC5)cc4c3)c(C)c(=O)c2c1] | 527 [M + H]⁺ |
| 92C | 2-(2-Ethyl-1,3- benzothiazol-6-yl)- 8-[(1R)-1- hydroxyethyl]-3,6- dimethyl- chromen-4-one | 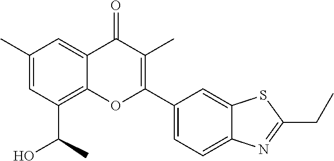 [SMILES: CCc1nc2ccc(-c3oc4c([C@@H](C)O)cc(C)cc4c(=O)c3C)cc2s1] | 380 [M + H]⁺ |
| 93C | 2-(2-Ethyl-1,3- benzoxazol-6-yl)- 8-[(1R)-1-hydroxyethyl]-3,6- dimethyl-chromen-4-one | 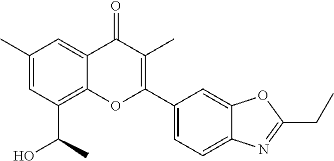 [SMILES: CCc1nc2ccc(-c3oc4c([C@@H](C)O)cc(C)cc4c(=O)c3C)cc2o1] | 364 [M + H]⁺ |
| 94C | 8-[(1S)-1- Hydroxyethyl]-3,6- dimethyl-2-[1-[(1- tetrahydropyran-2- yloxycyclopropyl) methyl]pyrazol- 4-yl]chromen-4-one | 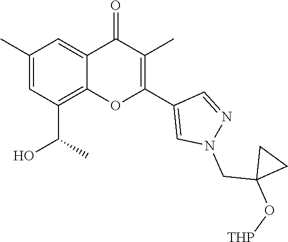 | 439 [M + H]⁺ |
| 95C | 8-[(1R)-1-[(6- Chloro-2-fluoro-3- pyridyl)oxy]ethyl]- 3,6-dimethyl- 2-(1H-pyrazol-5- yl)chromen-4- one | 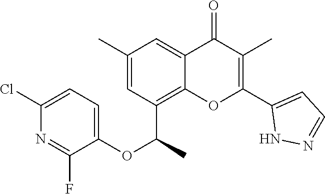 [SMILES: Cc1cc([C@@H](C)Oc2ccc(Cl)nc2F)c2oc(-c3ccn[nH]3)c(C)c(=O)c2c1] | 414 [M + H]⁺ |
| 96C | 3-chloro-8-[(1S)-1- Hydroxyethyl]- 6-methyl-2-(1- methylpyrazol-4- yl)chromen-4- one | 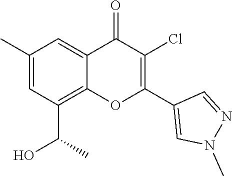 [SMILES: Cc1cc([C@H](C)O)c2oc(-c3cnn(C)c3)c(Cl)c(=O)c2c1] | 319 [M + H]⁺ |
| 97C | 8-[(1S)-1- Hydroxyethyl]-2-[1-(2- hydroxy-2-methyl- propyl)pyrazol- 4-yl]-3,6-dimethyl- chromen-4-one | 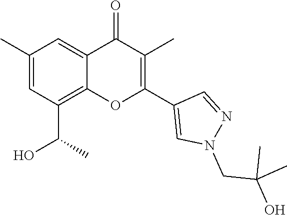 [SMILES: Cc1cc([C@H](C)O)c2oc(-c3cnn(CC(C)(C)O)c3)c(C)c(=O)c2c1] | 357 [M + H]⁺ |
| 98C | 8-[(1R)-1- Hydroxyethyl]-3,6- dimethyl-2-(1- methylpyrazol-4- yl)chromen-4-one | 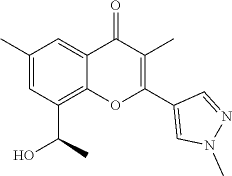 [SMILES: Cc1cc([C@@H](C)O)c2oc(-c3cnn(C)c3)c(C)c(=O)c2c1] | 299 [M + H]⁺ |

### Intermediate 99C: 6-Chloro-3-[(1R)-1-[2-(2-ethyl-1,3-benzothiazol-6-yl)-3,6-dimethyl-4-oxo-chromen-8-yl]ethoxy]pyridine-2-carbonitrile

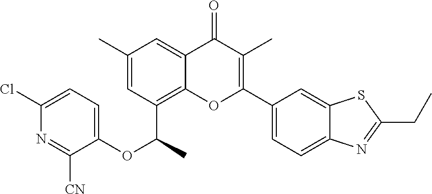 [SMILES: CCc1nc2ccc(-c3oc4c([C@@H](C)Oc5ccc(Cl)nc5C#N)cc(C)cc4c(=O)c3C)cc2s1]

To a 0° C. mixture of 2-(2-ethyl-1,3-benzothiazol-6-yl)-8-[(1R)-1-hydroxyethyl]-3,6-dimethyl-chromen-4-one (0.160 g, 0.422 mmol) in THE (10 mL) was added sodium hydride (60%; 0.202 g, 8.44 mmol) under nitrogen. The mixture was stirred at 0° C. for 40 min, then 6-chloro-3-fluoro-pyridine-2-carbonitrile (0.198 g, 1.27 mmol) was added. The reaction mixture was stirred for 1 h at rt, cooled to 0° C., and quenched with ice/water (50 mL). The resulting mixture was extracted with EtOAc (3×50 mL). The combined organic layers were washed with saturated aqueous sodium chloride (2×50 mL), dried over anhydrous sodium sulfate, and filtered. The filtrate was concentrated under reduced pressure to give the crude title compound as a brown solid. MS ES+ m z 516 [M+H]⁺.

### Intermediate 100C: 6-Chloro-3-[(1R)-1-[2-(2-ethyl-1,3-benzoxazol-6-yl)-3,6-dimethyl-4-oxo-chromen-8-yl]ethoxy]pyridine-2-carbonitrile

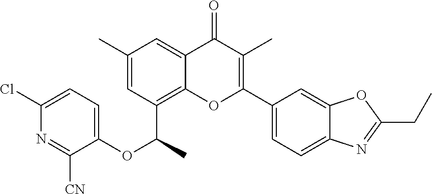 [SMILES: CCc1nc2ccc(-c3oc4c([C@@H](C)Oc5ccc(Cl)nc5C#N)cc(C)cc4c(=O)c3C)cc2o1]

2-(2-Ethyl-1,3-benzoxazol-6-yl)-8-[(1R)-1-hydroxyethyl]-3,6-dimethyl-chromen-4-one was used in a manner analogous to the method of preparation of 6-chloro-3-[(1R)-1-[2-(2-ethyl-1,3-benzothiazol-6-yl)-3,6-dimethyl-4-oxo-chromen-8-yl]ethoxy]pyridine-2-carbonitrile to afford the title compound (0.110 g, 27%). MS ES+ m/z 500 [M+H]⁺.

### Intermediate 57B: 8-[(1S)-1-Hydroxyethyl]-6-methyl-2-(1-methylpyrazol-4-yl)chromen-4-one

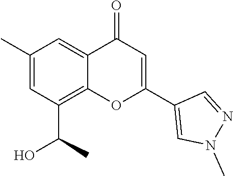 [SMILES: Cc1cc([C@@H](C)O)c2oc(-c3cnn(C)c3)cc(=O)c2c1]

8-Acetyl-6-methyl-2-(1-methylpyrazol-4-yl)chromen-4-one was used in a manner analogous to the method of preparation of Intermediate 4A to afford the title compound (1.24 g, 63%). MS ES+m/z 285 [M+H]⁺.

### Intermediate 58B: 3-Bromo-8-[(1S)-1-hydroxyethyl]-6-methyl-2-(1-methylpyrazol-4-yl)chromen-4-one

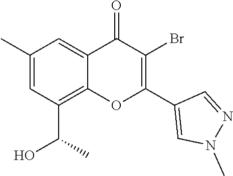 [SMILES: Cc1cc([C@H](C)O)c2oc(-c3cnn(C)c3)c(Br)c(=O)c2c1]

A mixture of 8-[(1S)-1-hydroxyethyl]-6-methyl-2-(1-methylpyrazol-4-yl)chromen-4-one (1.02 g, 3.59 mmol) and N-bromosuccinimide (0.798 g, 4.48 mmol) in ACN (20 mL) was stirred at rt for 1.5 h. The reaction mixture was filtered and the solids were washed with acetonitrile and water. The solids were dried under reduced pressure to give the title compound (0.870 g, 80% purity). MS ES+m/z 363,365 [M+H]⁺.

### Intermediate 59B: 8-[(1S)-1-Hydroxyethyl]-6-methyl-2-(1-methylpyrazol-4-yl)-4-oxo-chromene-3-carbonitrile

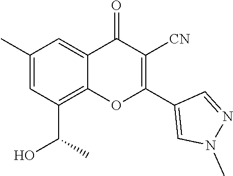 [SMILES: Cc1cc([C@H](C)O)c2oc(-c3cnn(C)c3)c(C#N)c(=O)c2c1]

A mixture of 3-bromo-8-[(1S)-1-hydroxyethyl]-6-methyl-2-(1-methylpyrazol-4-yl)chromen-4-one (0.870 g, 80%, 1.92 mmol) and potassium cyanide (0.624 g, 9.58 mmol) in DMF (10 mL) was stirred at 70° C. for 20 h, then cooled and concentrated. The residue was diluted with isopropanol:chloroform (1:3) and water. The aqueous layer was removed. The organic phase was dried over Na₂SO₄, filtered, and concentrated under reduced pressure. The residue was purified by silica gel chromatography eluted with a gradient of 0 to 10% MeOH in DCM to give the title compound (0.519 g, 88%). MS ES+ m/z 310 [M+H]⁺.

### Intermediate 11: 8-[(1S)-1-Chloroethyl]-3,6-dimethyl-2-phenyl-chromen-4-one

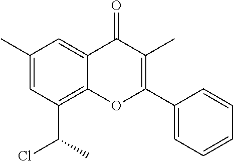 [SMILES: Cc1cc([C@H](C)Cl)c2oc(-c3ccccc3)c(C)c(=O)c2c1]

A flask equipped with overhead stirring was charged with 8-[(1R)-1-hydroxyethyl]-3,6-dimethyl-2-phenyl-chromen-4-one (20.0 g, 68.0 mmol) and cyclopentyl methyl ether (200 mL). Added 2,4,6-trichloro[1,3,5]triazine (12.5 g, 23.8 mmol) followed by DMF (7.9 mL, 102 mmol) and stirred overnight at rt. The reaction was treated slowly with 2M aqueous NaOH (100 mL) and allowed to stir for 10 min. The reaction was transferred to a separatory funnel and the organic layer removed. The organic layer was diluted with water (100 mL) and saturated aqueous NaHCO₃ (100 mL). After removal of the aqueous layer, the organic layer was washed with 5% aqueous LiCl. The organic layer was transferred to a flask and solvent swapped with IPA by charging the flask with 200 mL of IPA and concentrating down to 100 mL three times. The slurry was then warmed to 45° C. and stirred at that temperature for 2 h and allowed to cool to rt. The material was treated with 80 mL of water via syringe pump over 4 h and the reaction aged overnight. The product (18.4 g, 87%) was collected by filtration, washed with 40 mL of 1:1 IPA/water and dried at 45° C.

The following compound in Table 14 was made in a similar way as described for 8-[(1S)-1-chloroethyl]-3,6-dimethyl-2-phenyl-chromen-4-one.

TABLE 14

| Interm | Chemical | | MS ES+ |
|---|---|---|---|
| # | Name | Structure | m/z |
| 12 | 8-[(1S)-1- Chloroethyl]- 3,6-dimethyl- 2-(3- pyridyl) chromen- 4-one | 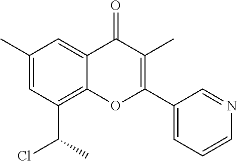 [SMILES: Cc1cc([C@H](C)Cl)c2oc(-c3cccnc3)c(C)c(=O)c2c1] | 314 [M + H]⁺ |

### Intermediate 13: 6-Chloro-3-[(1R)-1-(3,6-dimethyl-4-oxo-2-phenyl-chromen-8-yl)ethoxy]pyridine-2-carbonitrile

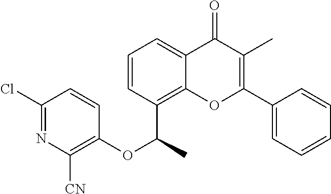 [SMILES: Cc1c(-c2ccccc2)oc2c([C@@H](C)Oc3ccc(Cl)nc3C#N)cccc2c1=O]

To a solution of 8-[(1S)-1-hydroxyethyl]-3,6-dimethyl-2-phenyl-chromen-4-one (0.200 g, 0.679 mmol), 6-chloro-3-hydroxy-pyridine-2-carbonitrile (0.196 g, 1.27 mmol), and triphenylphosphine (0.333 g, 1.27 mmol) in THE (6 mL) was added diisopropyl azodicarboxylate (0.257 g, 1.27 mmol) at 0° C. After 2 h, the reaction was concentrated under reduced pressure. The residue was purified by silica gel chromatography eluted with a gradient of 0 to 100% EtOAc in hexanes to give the title compound (0.300 g, 90% purity, 92%). MS ES+ m/z 431 [M+H]⁺.

The following compounds in Table 15 were made in a similar way as described for 6-chloro-3-[(1R)-1-(3,6-dimethyl-4-oxo-2-phenyl-chromen-8-yl)ethoxy]pyridine-2-carbonitrile. Various methods were used to purify the compounds, which would be apparent to one skilled in the art.

TABLE 15

| | | | MS ES+ |
|---|---|---|---|
| Interm # | Chemical Name | Structure | m/z |
| 14 | 3-[(1R)-1-(3,6-Dimethyl-4-oxo-2- phenyl-chromen-8-yl)ethoxy]pyridine- 2-carbonitrile | 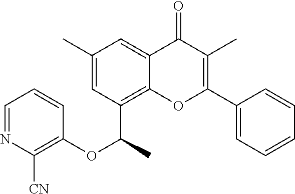 [SMILES: Cc1cc([C@@H](C)Oc2cccnc2C#N)c2oc(-c3ccccc3)c(C)c(=O)c2c1] | 397 [M + H]⁺ |
| 15 | 2-[(1R)-1-(3,6-Dimethyl-4-oxo-2- phenyl-chromen-8- yl)ethoxy]benzonitrile | 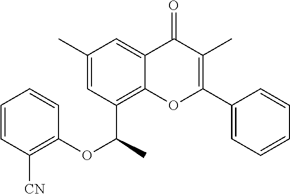 [SMILES: Cc1cc([C@@H](C)Oc2ccccc2C#N)c2oc(-c3ccccc3)c(C)c(=O)c2c1] | 396 [M + H]⁺ |
| 16 | 2-[(1R)-1-(3,6-Dimethyl-4-oxo-2- phenyl-chromen-8-yl)ethoxy]-6- fluoro-benzonitrile | 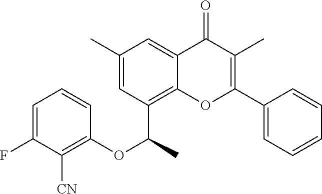 [SMILES: Cc1cc([C@@H](C)Oc2cccc(F)c2C#N)c2oc(-c3ccccc3)c(C)c(=O)c2c1] | 414 [M + H]⁺ |
| 17 | Methyl 6-Chloro-3-[(1R)-1-(3,6- dimethyl-4-oxo-2-phenyl-chromen-8- yl)ethoxy]pyridine-2-carboxylate | 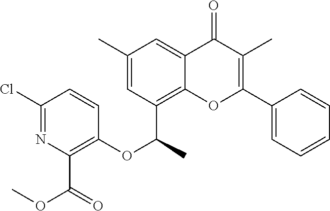 [SMILES: COC(=O)c1nc(Cl)ccc1O[C@H](C)c1cc(C)cc2c(=O)c(C)c(-c3ccccc3)oc12] | 464 [M + H]⁺ |
| 8A | 8-[(1R)-1-[(6-Chloro-2-fluoro-3- pyridyl)oxy]ethyl]-3,6-dimethyl-2-(2- methylindazol-5-yl)chromen-4-one | 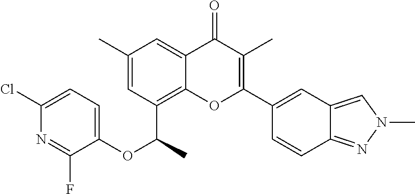 [SMILES: Cc1cc([C@@H](C)Oc2ccc(Cl)nc2F)c2oc(-c3ccc4nn(C)cc4c3)c(C)c(=O)c2c1] | 478 [M + H]⁺ |
| 21A | 8-[(1R)-1-[(2-Chloro-3- pyridyl)oxy]ethyl]-3,6-dimethyl-2- phenyl-chromen-4-one | 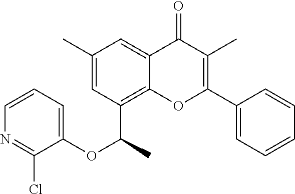 [SMILES: Cc1cc([C@@H](C)Oc2cccnc2Cl)c2oc(-c3ccccc3)c(C)c(=O)c2c1] | 406 [M + H]⁺ |
| 22A | 6-Chloro-3-[(1R)-1-[3,6-dimethyl-4- oxo-2-(2-pyridyl)chromen-8- yl]ethoxy]pyridine-2-carbonitrile | 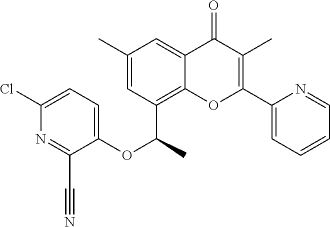 [SMILES: Cc1cc([C@@H](C)Oc2ccc(Cl)nc2C#N)c2oc(-c3ccccn3)c(C)c(=O)c2c1] | 432 [M + H]⁺ |
| 23A | 6-Chloro-3-[(1R)-1-[3,6-dimethyl-2- (1-methylpyrazol-4-yl)-2-oxo- chromen-8-yl]ethoxy]pyridine-2- carbonitrile | 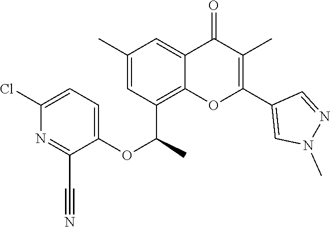 [SMILES: Cc1cc([C@@H](C)Oc2ccc(Cl)nc2C#N)c2oc(-c3cnn(C)c3)c(C)c(=O)c2c1] | 435 [M + H]⁺ |
| 24A | 6-Chloro-3-[(1R)-1-[3,6-dimethyl-4- oxo-2-(3-pyridyl)chromen-8- yl]ethoxy]pyridine-2-carbonitrile | 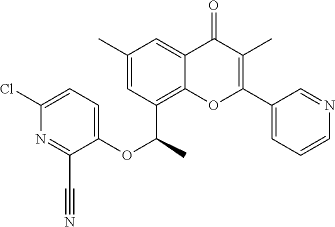 [SMILES: Cc1cc([C@@H](C)Oc2ccc(Cl)nc2C#N)c2oc(-c3cccnc3)c(C)c(=O)c2c1] | 432 [M + H]⁺ |
| 25A | 6-Chloro-3-[(1R)-1-[2-(2-fluoro-3- pyridyl)-3,6-dimethyl-4-oxo-chromen- 8-yl]ethoxy]pyridine-2-carbonitrile | 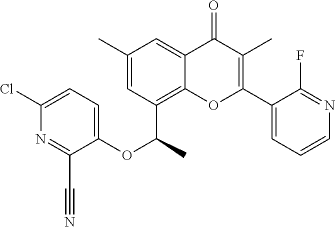 [SMILES: Cc1cc([C@@H](C)Oc2ccc(Cl)nc2C#N)c2oc(-c3cccnc3F)c(C)c(=O)c2c1] | 450 [M + H]⁺ |
| 26A | 6-Chloro-3-[(1R)-1-[2-(5-fluoro-3- pyridyl)-3,6-dimethyl-4-oxo-chromen- 8-yl]ethoxy]pyridine-2-carbonitrile | 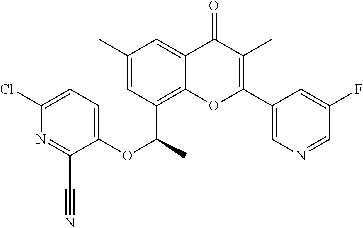 [SMILES: Cc1cc([C@@H](C)Oc2ccc(Cl)nc2C#N)c2oc(-c3cncc(F)c3)c(C)c(=O)c2c1] | 450 [M + H]⁺ |
| 27A | 6-Chloro-3-[(1R)-1-[2-(2,5-difluoro-3- pyridyl)-3,6-dimethyl-4-oxo-chromen- 8-yl]ethoxy]pyridine-2-carbonitrile | 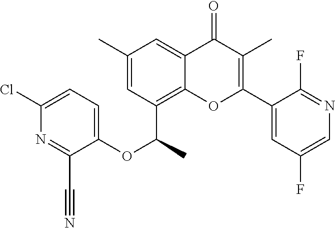 [SMILES: Cc1cc([C@@H](C)Oc2ccc(Cl)nc2C#N)c2oc(-c3cc(F)cnc3F)c(C)c(=O)c2c1] | 468 [M + H]⁺ |
| 28A | 2-[(1R)-1-[3,6-Dimethyl-2-(2- methylindazol-5-yl)-4-oxo-chromen-8- yl]ethoxy]benzonitrile | 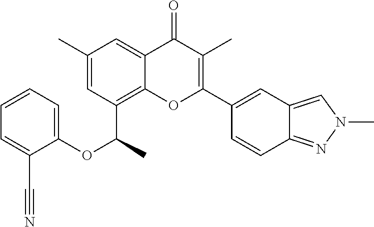 [SMILES: Cc1cc([C@@H](C)Oc2ccccc2C#N)c2oc(-c3ccc4nn(C)cc4c3)c(C)c(=O)c2c1] | [450 [M + H]⁺ |
| 29A | 3-[(1R)-1-[3,6-Dimethyl-2-(2- methylindazol-5-yl)-4-oxo-4-chromen-8- yl]ethoxy]pyridine-2-carbonitrile | 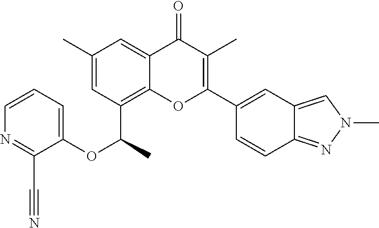 [SMILES: Cc1cc([C@@H](C)Oc2cccnc2C#N)c2oc(-c3ccc4nn(C)cc4c3)c(C)c(=O)c2c1] | 351 [M + H]⁺ |
| 30A | 6-Chloro-3-[(1R)-1-[3,6-dimethyl-2- (2-methylindazol-5-yl)-4-oxo- chromen-8-yl]ethoxy]pyridine-2- carbonitrile | 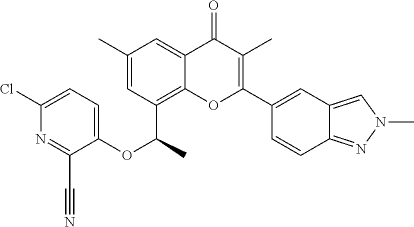 [SMILES: Cc1cc([C@@H](C)Oc2ccc(Cl)nc2C#N)c2oc(-c3ccc4nn(C)cc4c3)c(C)c(=O)c2c1] | 485 [M + H]⁺ |
| 31A | 8-[(1R)-1-[(6-Chloro-2-fluoro-3- pyridyl)oxy]ethyl]-3,6-dimethyl-2- phenyl-chromen-4-one | 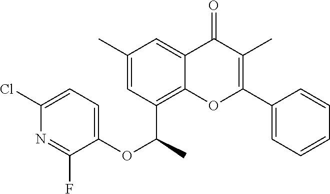 [SMILES: Cc1cc([C@@H](C)Oc2ccc(Cl)nc2F)c2oc(-c3ccccc3)c(C)c(=O)c2c1] | 424 [M + H]⁺ |
| 60B | 6-Chloro-3-[(1R)-1-[3,6-dimethyl-2- (2-methylthiazol-4-yl)-4-oxo-chromen- 8-yl]ethoxy]pyridine-2-carbonitrile | 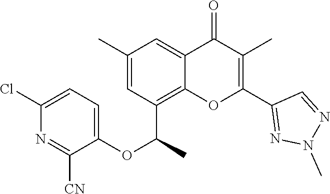 [SMILES: Cc1cc([C@@H](C)Oc2ccc(Cl)nc2C#N)c2oc(-c3cnn(C)n3)c(C)c(=O)c2c1] | 436 [M + H]⁺ |
| 61B | 6-Chloro-3-[(1R)-1-[2-(6,7-dihydro- 4H-pyrazolo[5,1-c][1,4]oxazin-3-yl)- 3,6-dimethyl-4-oxo-chromen-8- yl]ethoxy]pyridine-2-carbonitrile | 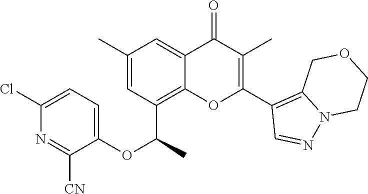 [SMILES: Cc1cc([C@@H](C)Oc2ccc(Cl)nc2C#N)c2oc(-c3cnn4c3COCC4)c(C)c(=O)c2c1] | 477 [M + H]⁺ |
| 62B | methyl 6-[(1R)-1-[3,6-Dimethyl-2-(1- methylpyrazol-4-yl)-4-oxo-chromen- 8-yl]ethoxy]-2,3-difluoro-benzoate | 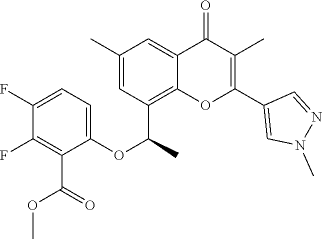 | 447 [M + H]⁺ |
| 63B | 8-[(1R)-1-[(6-Chloro-2-fluoro-3- pyridyl)oxy]ethyl]-3,6-dimethyl-2-(3- pyridyl)chromen-4-one | 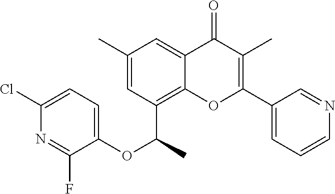 [SMILES: Cc1cc([C@@H](C)Oc2ccc(Cl)nc2F)c2oc(-c3cccnc3)c(C)c(=O)c2c1] | 425 [M + H]⁺ |
| 64B | methyl 3-[(1R)-1-[3,6-Dimethyl-2-(2- methyindazol-5-yl)-4-oxo-chromen-8- yl]ethoxy]pyridine-2-carboxylate | 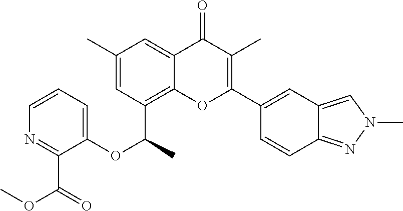 | 484 [M + H]⁺ |
| 65B | methyl 6-Chloro-3-[(1R)-1-[3,6- dimethyl-4-oxo-2-(2-pyridyl)chromen- 8-yl]ethoxy]pyridine-2-carboxylate | 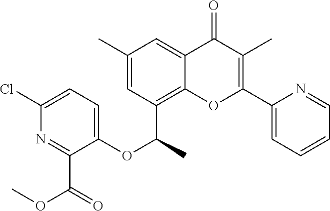 [SMILES: COC(=O)c1nc(Cl)ccc1O[C@H](C)c1cc(C)cc2c(=O)c(C)c(-c3ccccn3)oc12] | 465 [M + H]⁺ |
| 66B | methyl 6-Chloro-3-[(1R)-1-[2-(5- fluoro-3-pyridyl)-3,6-dimethyl-4-oxo- chromen-8-yl]ethoxy]pyridine-2- carboxylate | 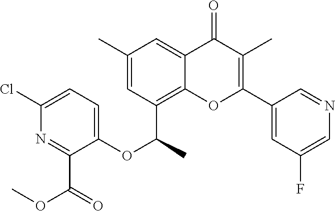 [SMILES: COC(=O)c1nc(Cl)ccc1O[C@H](C)c1cc(C)cc2c(=O)c(C)c(-c3cncc(F)c3)oc12] | 483 [M + H]⁺ |
| 67B | 2-[1-[2-[tert- Butyl(dimethylsilyl]oxyethyl]pyrazol- 4-yl]-8-[(1R)-1-[(6-chloro-2-fluoro-3- pyridyl)oxy]ethyl]-3,6-dimethyl chromen-4-one | 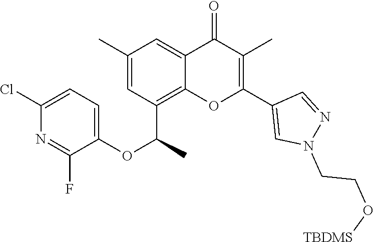 | 572 [M + H]⁺ |
| 68B | 8-[(1R)-1-[(6-Chloro-2-fluoro-3- pyridyl)oxy]ethyl]-2-cyclopropyl-3,6- dimethyl-chromen-4-one | 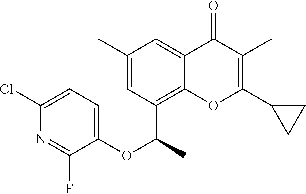 [SMILES: Cc1cc([C@@H](C)Oc2ccc(Cl)nc2F)c2oc(C3CC3)c(C)c(=O)c2c1] | 388 [M + H]⁺ |
| 69B | 6-Chloro-3-[(1R)-1-(2-cyclopropyl- 3,6-dimethyl-4-oxo-chromen-8- yl)ethoxy]pyridine-2-carbonitrile | 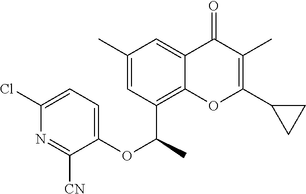 [SMILES: Cc1cc([C@@H](C)Oc2ccc(Cl)nc2C#N)c2oc(C3CC3)c(C)c(=O)c2c1] | 395 [M + H]⁺ |
| 70B | 6-Chloro-3-[1-[7-chloro-3,6-dimethyl- 4-oxo-2-(3-pyridyl)chromen-8- yl]ethoxy]pyridine-2-carbonitrile | 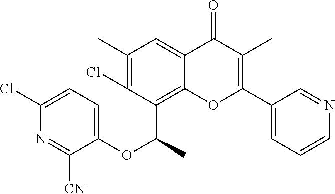 [SMILES: Cc1cc2c(=O)c(C)c(-c3cccnc3)oc2c([C@@H](C)Oc2ccc(Cl)nc2C#N)c1Cl] | 466 [M + H]⁺ |
| 101C | 6-Chloro-3-[(1R)-1-(2-furo[3,2- b]pyridin-6-yl-3,6-dimethyl-4-oxo- chromen-8-yl)ethoxy]pyridine-2- carbonitrile | 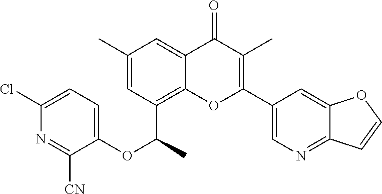 [SMILES: Cc1cc([C@@H](C)Oc2ccc(Cl)nc2C#N)c2oc(-c3cnc4ccoc4c3)c(C)c(=O)c2c1] | 472 [M + H]⁺ |
| 102C | 6-Bromo-3-[(1R)-1-[3,6-dimethyl-2- (2-methylindazol-5-yl)-4-oxo- chromen-8-yl]ethoxy]pyridine-2- carbonitrile | 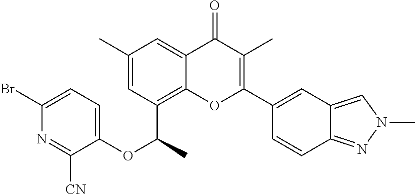 [SMILES: Cc1cc([C@@H](C)Oc2ccc(Br)nc2C#N)c2oc(-c3ccc4nn(C)cc4c3)c(C)c(=O)c2c1] | 531 [M + H]⁺ |
| 103C | 2-[(1R)-1-[3,6-Dimethyl-2-(2- methylindazol-5-yl)-4-oxo-chromen-8- yl]ethoxy]-5-fluoro-benzonitrile | 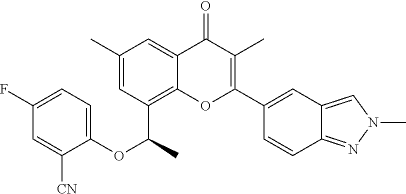 [SMILES: Cc1cc([C@@H](C)Oc2ccc(F)cc2C#N)c2oc(-c3ccc4nn(C)cc4c3)c(C)c(=O)c2c1] | 468 [M + H]⁺ |
| 104C | 2-[(1R)-1-[3,6-Dimethyl-2-(2- methylindazol-5-yl)-4-oxo-chromen-8- yl]ethoxy]-6-fluoro-benzonitrile | 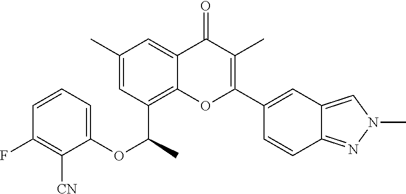 [SMILES: Cc1cc([C@@H](C)Oc2cccc(F)c2C#N)c2oc(-c3ccc4nn(C)cc4c3)c(C)c(=O)c2c1] | 468 [M + H]⁺ |
| 105C | 6-Chloro-3-[(1R)-1-[3,6-dimethyl-4- oxo-2-[1-[(1-tetrahydropyran-2- yloxycyclopropyl)methyl]pyrazol-4- yl]chromen-8-yl]ethoxy]pyridine-2- sulfonamide | 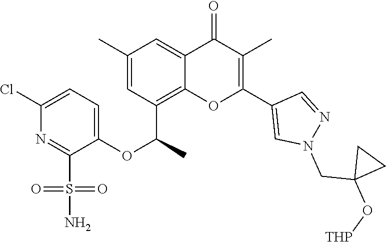 | 627 [M − H]⁻ |
| 106C | ethyl 2-[8-[(1R)-1-[(6-Chloro-2- fluoro-3-pyridyl)oxy]ethyl]-3,6- dimethyl-4-oxo-chromen-2- yl]cyclopropanecarboxylate | 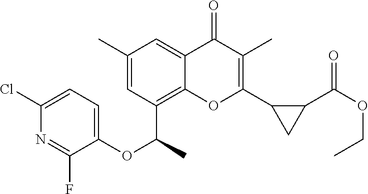 [SMILES: CCOC(=O)C1CC1c1oc2c([C@@H](C)Oc3ccc(Cl)nc3F)cc(C)cc2c(=O)c1C] | 460 [M + H]⁺ |
| 107C | ethyl 2-[1-[8-[(1R)-1-[(6-Chloro-2- sulfamoyl-3-pyridyl)oxy]ethyl]-3,6- dimethyl-4-oxo-chromen-2- yl]cyclopropyl]acetate | 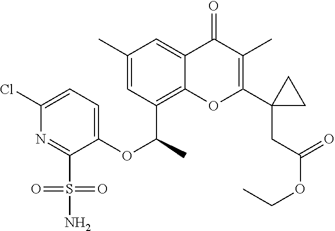 [SMILES: CCOC(=O)CC1(c2oc3c([C@@H](C)Oc4ccc(Cl)nc4S(N)(=O)=O)cc(C)cc3c(=O)c2C)CC1] | 535 [M + H]⁺ |

### Intermediate 18: 6-Chloro-3-[(1R)-1-(3,6-dimethyl-4-oxo-2-phenyl-chromen-8-yl)ethoxy]-N′-hydroxy-pyridine-2-carboxamidine

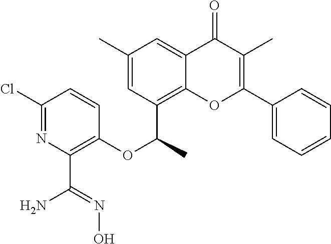 [SMILES: Cc1cc([C@@H](C)Oc2ccc(Cl)nc2/C(N)=N/O)c2oc(-c3ccccc3)c(C)c(=O)c2c1]

To a solution of 6-chloro-3-[(1R)-1-(3,6-dimethyl-4-oxo-2-phenyl-chromen-8-yl)ethoxy]pyridine-2-carbonitrile (0.588 g, 1.36 mmol) in EtOH (6 mL) was added triethylamine (0.38 mL, 2.73 mmol) and hydroxylamine hydrochloride (0.114 g, 1.64 mmol). The reaction was heated at 80° C. overnight and concentrated under reduced pressure. The residue was purified by reversed phase chromatography eluted with a gradient of 0 to 100% ACN in H₂O+0.1% formic acid. Fractions containing the desired product were combined, washed with saturated aqueous sodium chloride, and extracted with IPA:CHCl₃ (1:3). The combined extracts were dried over MgSO₄, filtered, and concentrated under reduced pressure to give the title compound (0.180 g, 28%). MS ES+ m/z 464 [M+H]⁺.

The following compounds in Table 16 were made in a similar way as described for 6-chloro-3-[(1R)-1-(3,6-dimethyl-4-oxo-2-phenyl-chromen-8-yl)ethoxy]-N′-hydroxy-pyridine-2-carboxamidine. Various methods were used to purify the compounds, which would be apparent to one skilled in the art.

TABLE 16

| | | | MS ES+ |
|---|---|---|---|
| Interm # | Chemical Name | Structure | m/z |
| 19 | 3-[(1R)-1-(3,6-Dimethyl- 4-oxo-2-phenyl- chromen-8-yl)ethoxy]- N′-hydroxy-pyridine-2- carboxamidine | 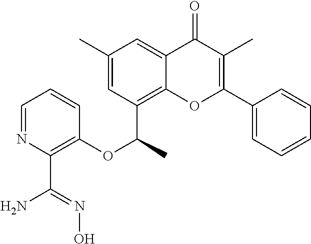 [SMILES: Cc1cc([C@@H](C)Oc2cccnc2/C(N)=N/O)c2oc(-c3ccccc3)c(C)c(=O)c2c1] | 430 [M + H]⁺ |
| 20 | 2-[(1R)-1-(3,6-Dimethyl- 4-oxo-2-phenyl- chromen-8-yl)ethoxy]- N′-hydroxy-benzamidine | 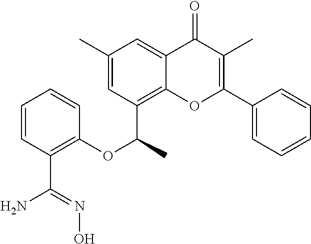 [SMILES: Cc1cc([C@@H](C)Oc2ccccc2/C(N)=N/O)c2oc(-c3ccccc3)c(C)c(=O)c2c1] | 429 [M + H]⁺ |
| 21 | 2-[(1R)-1-(3,6-Dimethyl- 4-oxo-2-phenyl- chromen-8-yl)ethoxy]-6- fluoro-N′-hydroxy- benzamidine | 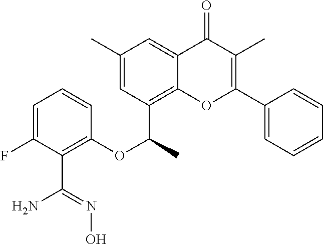 [SMILES: Cc1cc([C@@H](C)Oc2cccc(F)c2/C(N)=N/O)c2oc(-c3ccccc3)c(C)c(=O)c2c1] | 447 [M + H]⁺ |
| 32A | 6-Chloro-3-[(1R)-1-[3,6- dimethyl-4-oxo-2-(2- pyridyl)chromen-8- yl]ethoxy]-N′-hydroxy- pyridine-2- carboxamidine | 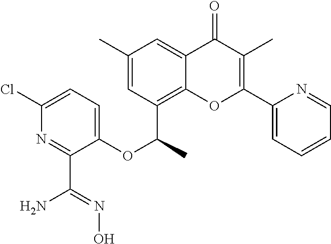 [SMILES: Cc1cc([C@@H](C)Oc2ccc(Cl)nc2/C(N)=N/O)c2oc(-c3ccccn3)c(C)c(=O)c2c1] | 465 [M + H]⁺ |
| 33A | 6-Chloro-3-[(1R)-1-[3,6- dimethyl-2-(1- methylpyrazol-4-yl)-4- oxo-chromen-8- yl]ethoxy]-N′-hydroxy- pyridine-2- carboxamidine | 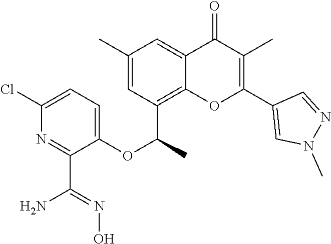 [SMILES: Cc1cc([C@@H](C)Oc2ccc(Cl)nc2/C(N)=N/O)c2oc(-c3cnn(C)c3)c(C)c(=O)c2c1] | 468 [M + H]⁺ |
| 34A | 6-Chloro-3-[(1R)-1-[3,6- dimethyl-4-oxo-2-(3- pyridyl)chromen-8- yl]ethoxy]-N′-hydroxy- pyridine-2- carboxamidine | 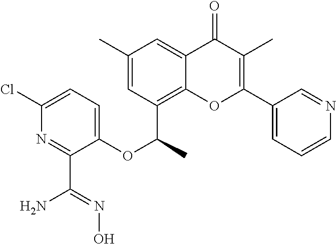 [SMILES: Cc1cc([C@@H](C)Oc2ccc(Cl)nc2/C(N)=N/O)c2oc(-c3cccnc3)c(C)c(=O)c2c1] | 465 [M + H]⁺ |
| 35A | 6-Chloro-3-[(1R)-1-[2- (2-fluoro-3-pyridyl)-3,6- dimethyl-4-oxo- chromen-8-yl]ethoxy]- N′-hydroxy-pyridine-2- carboxamidine | 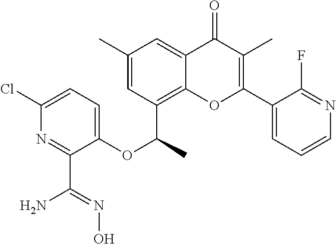 [SMILES: Cc1cc([C@@H](C)Oc2ccc(Cl)nc2/C(N)=N/O)c2oc(-c3cccnc3F)c(C)c(=O)c2c1] | 483 [M + H]⁺ |
| 36A | 6-Chloro-3-[(1R)-1-[2- (5-fluoro-3-pyridyl)-3,6- dimethyl-4-oxo- chromen-8-yl]ethoxy]- N′-hydroxy-pyridine-2- carboxamidine | 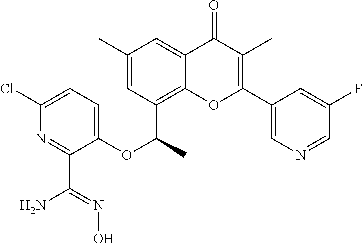 [SMILES: Cc1cc([C@@H](C)Oc2ccc(Cl)nc2/C(N)=N/O)c2oc(-c3cncc(F)c3)c(C)c(=O)c2c1] | 483 [M + H]⁺ |
| 37A | 6-Chloro-3-[(1R)-1-[2- (2,5-difluoro-3-pyridyl)- 3,6-dimethyl-4-oxo- chromen-8-yl]ethoxy]- N′-hydroxy-pyridine-2- carboxamidine | 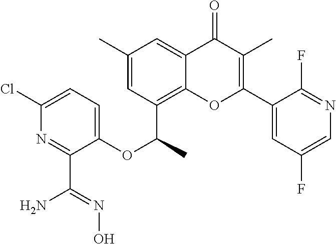 [SMILES: Cc1cc([C@@H](C)Oc2ccc(Cl)nc2/C(N)=N/O)c2oc(-c3cc(F)cnc3F)c(C)c(=O)c2c1] | 501 [M + H]⁺ |
| 38A | 6-Chloro-3-[(1R)-1-[2- (3-fluoro-2-pyridyl)-3,6- dimethyl-4-oxo- chromen-8-yl]ethoxy]- N′-hydroxy-pyridine-2- carboxamidine | 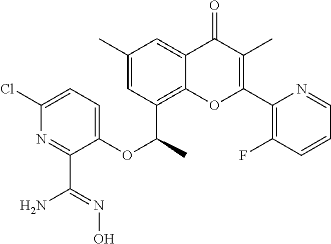 [SMILES: Cc1cc([C@@H](C)Oc2ccc(Cl)nc2/C(N)=N/O)c2oc(-c3ncccc3F)c(C)c(=O)c2c1] | 483 [M + H]⁺ |
| 39A | 6-Chloro-3-[(1R)-1-[2- (6-fluoro-2-pyridyl)-3,6- dimethyl-4-oxo- chromen-8-yl]ethoxy]- N′-hydroxy-pyridine-2- carboxamidine | 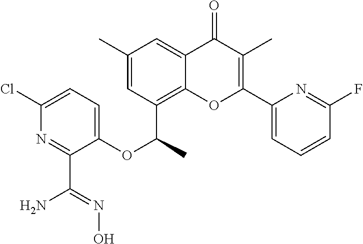 [SMILES: Cc1cc([C@@H](C)Oc2ccc(Cl)nc2/C(N)=N/O)c2oc(-c3cccc(F)n3)c(C)c(=O)c2c1] | 483 [M + H]⁺ |
| 71B | 6-Chloro-3-[(1R)-1-[3,6- dimethyl-2-(2- methylindazol-5-yl)-4- oxo-chromen-8- yl]ethoxy]-N′-hydroxy- pyridine-2- carboxamidine | 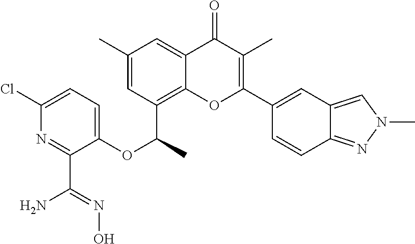 [SMILES: Cc1cc([C@@H](C)Oc2ccc(Cl)nc2/C(N)=N/O)c2oc(-c3ccc4nn(C)cc4c3)c(C)c(=O)c2c1] | 516 [M − H]⁻ |
| 72B | 6-Chloro-3-[(1R)-1-[3,6- dimethyl-2-(2- methyltriazol-4-yl)-4- oxo-chromen-8- yl]ethoxy]-N′-hydroxy- pyridine-2- carboxamidine | 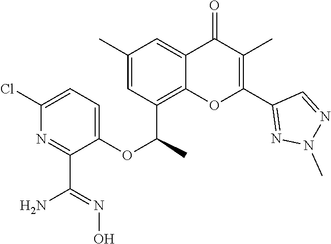 [SMILES: Cc1cc([C@@H](C)Oc2ccc(Cl)nc2/C(N)=N/O)c2oc(-c3cnn(C)n3)c(C)c(=O)c2c1] | 469 [M + H]⁺ |
| 73B | 6-Chloro-3-[(1R)-1-[2- ([1,3]dioxolo[4,5- b]pyridin-6-yl)-3,6- dimethyl-4-oxo- chromen-8-yl]ethoxy]- N′-hydroxy-pyridine-2- carboxamidine | 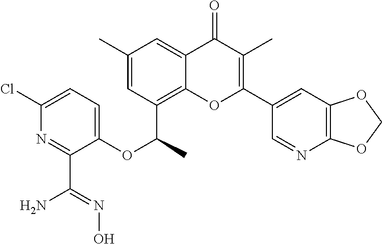 [SMILES: Cc1cc([C@@H](C)Oc2ccc(Cl)nc2/C(N)=N/O)c2oc(-c3cnc4c(c3)OCO4)c(C)c(=O)c2c1] | 509 [M + H]⁺ |
| 74B | 6-Chloro-3-[(1R)-1-[2- (6,7-dihydro-4H- pyrazolo[5,1- c][1,4]oxazin-3-yl)-3,6- dimethyl-4-oxo- chromen-8-yl]ethoxy]- N′-hydroxy-pyridine-2- carboxamidine | 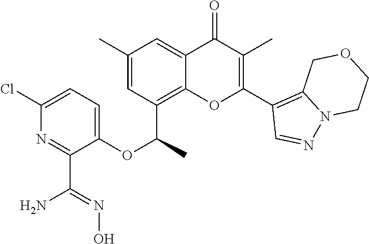 [SMILES: Cc1cc([C@@H](C)Oc2ccc(Cl)nc2/C(N)=N/O)c2oc(-c3cnn4c3COCC4)c(C)c(=O)c2c1] | 510 [M + H]⁺ |
| 75B | 6-Chloro-3-[(1R)-1-[2- (2,2-difluoro-1,3- benzodioxol-5-yl)-3,6- dimethyl-4-oxo- chromen-8-yl]ethoxy]- N′-hydroxy-pyridine-2- carboxamidine | 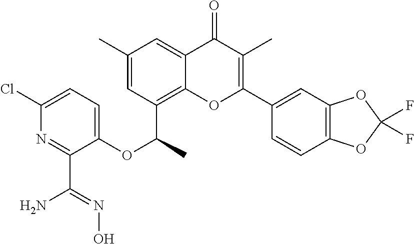 [SMILES: Cc1cc([C@@H](C)Oc2ccc(Cl)nc2/C(N)=N/O)c2oc(-c3ccc4c(c3)OC(F)(F)O4)c(C)c(=O)c2c1] | 542 [M − H]⁻ |
| 76B | 6-Chloro-3-[(1R)-1-[2- (2,3-dihydro- [1,4]dioxino[2,3- [b]pyridin-7-yl)-3,6- dimethyl-4-oxo- chromen-8-yl]ethoxy]- N′-hydroxy-pyridine-2- carboxamidine | 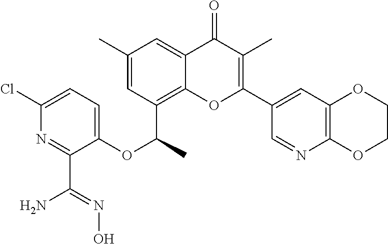 [SMILES: Cc1cc([C@@H](C)Oc2ccc(Cl)nc2/C(N)=N/O)c2oc(-c3cnc4c(c3)OCCO4)c(C)c(=O)c2c1] | 523 [M + H]⁺ |
| 77B | 6-Chloro-3-[(1R)-1-[3,6- dimethyl-2-(4-methyl- 2,3-dihydro-1,4- benzoxazin-7-yl)-4-oxo- chromen-8-yl]ethoxy]- N′-hydroxy-pyridine-2- carboxamidine | 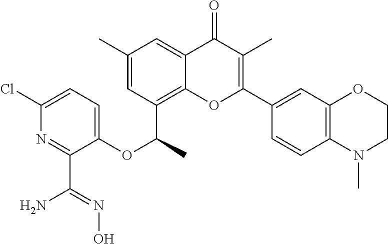 [SMILES: Cc1cc([C@@H](C)Oc2ccc(Cl)nc2/C(N)=N/O)c2oc(-c3ccc4c(c3)OCCN4C)c(C)c(=O)c2c1] | 533 [M − H]⁻ |
| 78B | 6-Chloro-3-[(1R)-1-[3,6- dimethyl-2-(4-methyl- 2,3-dihydropyrido[3,2- [b][1,4]oxazin-7-yl)-4- oxo-chromen-8- yl]ethoxy]-N′-hydroxy- pyridine-2- carboxamidine | 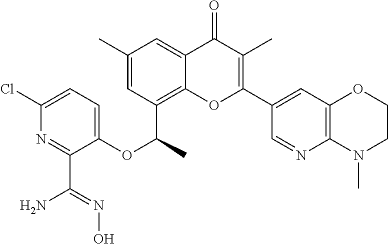 [SMILES: Cc1cc([C@@H](C)Oc2ccc(Cl)nc2/C(N)=N/O)c2oc(-c3cnc4c(c3)OCCN4C)c(C)c(=O)c2c1] | 536 [M + H]⁺ |
| 79B | 6-Chloro-3-[(1R)-1-[3,6- dimethyl-2-(1,5- naphthyridin-3-yl)-4- oxo-chromen-8- yl]ethoxy]-N′-hydroxy- pyridine-2- carboxamidine | 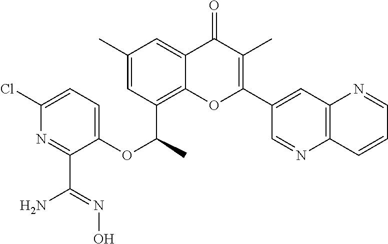 [SMILES: Cc1cc([C@@H](C)Oc2ccc(Cl)nc2/C(N)=N/O)c2oc(-c3cnc4cccnc4c3)c(C)c(=O)c2c1] | 516 [M + H]⁺ |
| 80B | 6-Chloro-3-[(1R)-1-[2- (5,6-dihydro-4H- pyrrolo[2,1-e]pyrazol-3- yl)-3,6-dimethyl-4-oxo- chromen-8-yl]ethoxy]- N′-hydroxy-pyridine-2- carboxamidine | 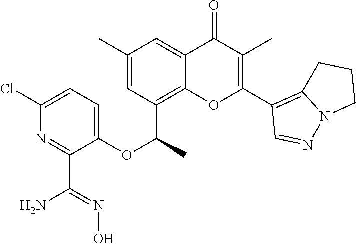 [SMILES: Cc1cc([C@@H](C)Oc2ccc(Cl)nc2/C(N)=N/O)c2oc(-c3cnn4c3CCC4)c(C)c(=O)c2c1] | 494 [M + H]⁺ |
| 81B | 3-[(1R)-1-[3,6-Dimethyl- 2-(2-methylindazol-5-yl)- 4-oxo-chromen-8- yl]ethoxy]-6-fluoro-N′- hydroxy-pyridine-2- carboxamidine | 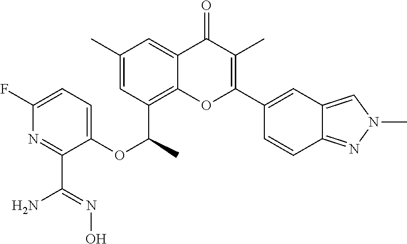 [SMILES: Cc1cc([C@@H](C)Oc2ccc(F)nc2/C(N)=N/O)c2oc(-c3ccc4nn(C)cc4c3)c(C)c(=O)c2c1] | 502 [M + H]⁺ |
| 82B | 6-Chloro-3-[(1R)-1-(3,6- dimethyl-4-oxo-2- thiazolo[5,4-b]pyridin-6- yl-chromen-8-yl)ethoxy]- N′-hydroxy-pyridine-2- carboxamidine | 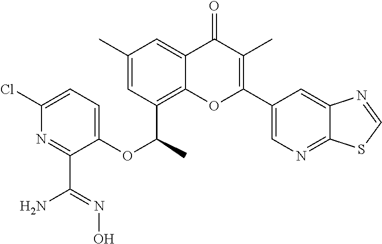 [SMILES: Cc1cc([C@@H](C)Oc2ccc(Cl)nc2/C(N)=N/O)c2oc(-c3cnc4scnc4c3)c(C)c(=O)c2c1] | 522 [M + H]⁺ |
| 83B | 6-Chloro-3-[(1R)-1-(2- cyclopropyl-3,6- dimethyl-4-oxo- chromen-8-yl)ethoxy]- N′-hydroxy-pyridine-2- carboxamidine | 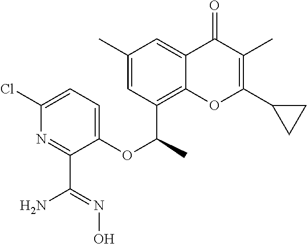 [SMILES: Cc1cc([C@@H](C)Oc2ccc(Cl)nc2/C(N)=N/O)c2oc(C3CC3)c(C)c(=O)c2c1] | 426 [M − H]⁻ |
| 108C | 6-Chloro-3-[(1R)-1-[3,6- dimethyl-2-(2- methylpyrazolo[4,3- b]pyridin-5-yl)-4-oxo- chromen-8-yl]ethoxy]- N′-hydroxy-pyridine-2- carboxamidine | 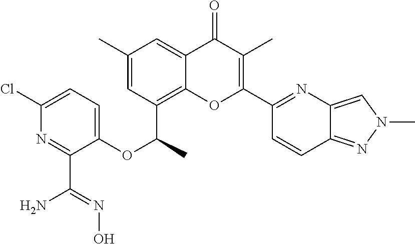 [SMILES: Cc1cc([C@@H](C)Oc2ccc(Cl)nc2/C(N)=N/O)c2oc(-c3ccc4nn(C)cc4n3)c(C)c(=O)c2c1] | 519 [M + H]⁺ |
| 109C | 6-Chloro-3-[(1R)-1-[3,6- dimethyl-2-(1- methylpyrazolo[3,4- b]pyridin-6-yl)-4-oxo- chromen-8-yl]ethoxy]- N′-hydroxy-pyridine-2- carboxamidine | 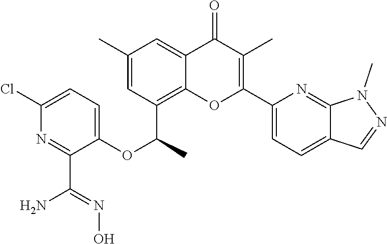 [SMILES: Cc1cc([C@@H](C)Oc2ccc(Cl)nc2/C(N)=N/O)c2oc(-c3ccc4cnn(C)c4n3)c(C)c(=O)c2c1] | 519 [M + H]⁺ |
| 110C | 6-Chloro-3-[(1R)-1-(3,6- dimethyl-4-oxo-2- pyrazin-2-yl-chromen-8- yl)ethoxy]-N′-hydroxy- pyridine-2- carboxamidine | 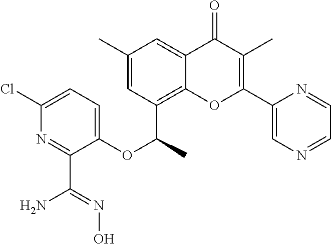 [SMILES: Cc1cc([C@@H](C)Oc2ccc(Cl)nc2/C(N)=N/O)c2oc(-c3cnccn3)c(C)c(=O)c2c1] | 466 [M + H]⁺ |
| 111C | 6-Chloro-3-[(1R)-1-[3,6- dimethyl-2-(2- methylimidazo[1,2- a]pyridin-6-yl)-4-oxo- chromen-8-yl]ethoxy]- N′-hydroxy-pyridine-2- carboxamidine | 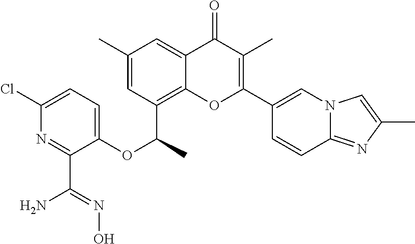 [SMILES: Cc1cc([C@@H](C)Oc2ccc(Cl)nc2/C(N)=N/O)c2oc(-c3ccc4nc(C)cn4c3)c(C)c(=O)c2c1] | 518 [M + H]⁺ |
| 112C | 6-Chloro-N′-hydroxy-3- [(1R)-1-[2-(2- isopropoxypyrimidin-5- yl)-3,6-dimethyl-4-oxo- chromen-8- yl]ethoxy]pyridine-2- carboxamidine | 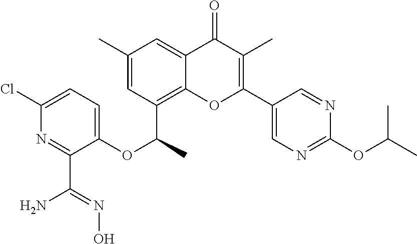 [SMILES: Cc1cc([C@@H](C)Oc2ccc(Cl)nc2/C(N)=N/O)c2oc(-c3cnc(OC(C)C)nc3)c(C)c(=O)c2c1] | 524 [M + H]⁺ |
| 113C | 6-Chloro-3-[(1R)-1-(2- furo[3,2-b]pyridin-6-yl- 3,6-dimethyl-4-oxo- chromen-8-yl)ethoxy]- N′-hydroxy-pyridine-2- carboxamidine | 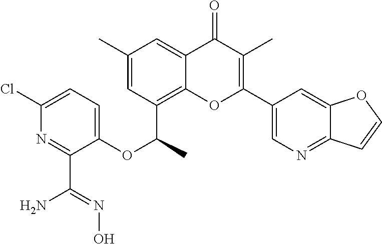 [SMILES: Cc1cc([C@@H](C)Oc2ccc(Cl)nc2/C(N)=N/O)c2oc(-c3cnc4ccoc4c3)c(C)c(=O)c2c1] | 505 [M + H]⁺ |

### Intermediate 841B: 2-[1-[(2S)-2-[tert-Butyl(dimethyl)silyl]oxypropyl]pyrazol-4-yl]-8-[(1R)-1-[(6-chloro-2-fluoro-3-pyridyl)oxy]ethyl]-3,6-dimethyl-chromen-4-one

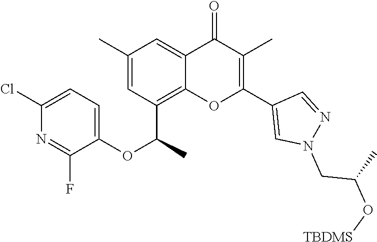

A solution of 8-[(1R)-1-[(6-chloro-2-fluoro-3-pyridyl)oxy]ethyl]-2-[1-[(2S)-2-hydroxypropyl]pyrazol-4-yl]-3,6-dimethyl-chromen-4-one (0.278 g, 0.589 mmol) in DCM (6 mL) was treated with tert-butylchlorodimethylsilane (0.133 g, 0.884 mmol), 1,8-diazabicyclo[5.4.0]undec-7-ene (0.179 g, 1.18 mmol) and 4-dimethylaminopyridine (0.072 g, 0.589 mmol). The reaction was stirred at rt for 1 h, then treated with tert-butylchlorodimethylsilane (0.133 g, 0.884 mmol) and 1,8-diazabicyclo[5.4.0]undec-7-ene (0.179 g, 1.18 mmol). The reaction was stirred at rt for 1 h, then diluted with EtOAc and washed with saturated aqueous sodium chloride. The aqueous layer was back extracted with EtOAc. The combined organic phases were dried over Na₂SO₄, filtered, and concentrated under reduced pressure to give the title compound (0.35 g, 91%) as a tan gel. MS ES+ m/z 586 [M+H]⁺.

### Intermediate 40A: 8-[(1R)-1-[(2-Benzylsulfanyl-6-chloro-3-pyridyl)oxy]ethyl]-3,6-dimethyl-2-phenyl-chromen-4-one

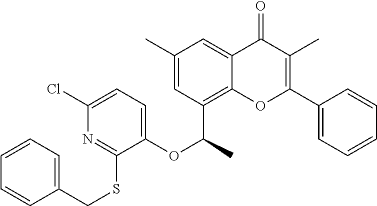 [SMILES: Cc1cc([C@@H](C)Oc2ccc(Cl)nc2SCc2ccccc2)c2oc(-c3ccccc3)c(C)c(=O)c2c1]

A suspension of sodium hydride (1.08 g, 60%, 26.90 mmol) in THE (30 mL) at 0° C. was treated with benzyl mercaptan (3.34 g, 26.90 mmol). The bath was removed and the reaction allowed to stir for 20 min. The reaction was cooled to 0° C. and treated with 8-[(1R)-1-[(6-chloro-2-fluoro-3-pyridyl)oxy]ethyl]-3,6-dimethyl-2-phenyl-chromen-4-one (2.28 g, 26.90 mmol) dissolved in THE (3 mL). The reaction was allowed to warm to rt and stirred for 2 h. The reaction was diluted with EtOAc and washed with water. The organic layer was collected, dried over Na₂SO₄, filtered, and concentrated under reduced pressure. The residue was purified by silica gel chromatography eluted with 000 to 40% EtOAc in heptane to give the title compound (1.35 g, 48%) as a white solid. ES-MS m/z 528 (M+H).

The following compounds in Table 17 were made in a similar way as described for 8-[(1R)-1-[(2-benzylsulfanyl-6-chloro-3-pyridyl)oxy]ethyl]-3,6-dimethyl-2-phenyl-chromen-4-one. Various methods were used to purify the compounds, which would be apparent to one skilled in the art.

TABLE 17

| | | | MS ES+ |
|---|---|---|---|
| Interm # | Chemical Name | Structure | m/z |
| 42A | 8-[(1R)-1-[(2-Benzylsulfanyl-6-chloro- 3-pyridyl)oxy]ethyl]-3,6-dimethyl-2-(1- methylpyrazol-4-yl)chromen-4-one | 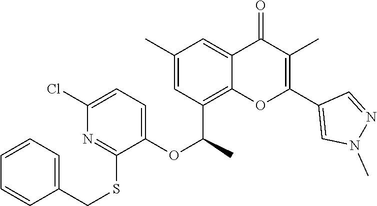 [SMILES: Cc1cc([C@@H](C)Oc2ccc(Cl)nc2SCc2ccccc2)c2oc(-c3cnn(C)c3)c(C)c(=O)c2c1] | 532 [M + H]⁺ |
| 43A | 8-[(1R)-1-[(2-Benzylsulfanyl-6-chloro- 3-pyridyl)oxy]ethyl]-2- ([1,3]dioxolo[4,5-b]pyridin-6-yl)-3,6- dimethyl-chromen-4-one | 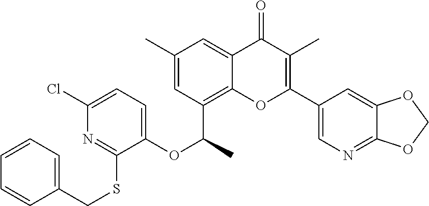 [SMILES: Cc1cc([C@@H](C)Oc2ccc(Cl)nc2SCc2ccccc2)c2oc(-c3cnc4c(c3)OCO4)c(C)c(=O)c2c1] | 573 [M + H]⁺ |
| 44A | 8-[(1R)-1-[(2-Benzylsulfanyl-6-chloro- 3-pyridyl)oxy]ethyl]-3,6-dimethyl-2-(2- methylindazol-5-yl)chromen-4-one | 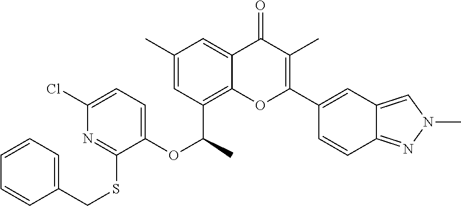 [SMILES: Cc1cc([C@@H](C)Oc2ccc(Cl)nc2SCc2ccccc2)c2oc(-c3ccc4nn(C)cc4c3)c(C)c(=O)c2c1] | 582 [M + H]⁺ |
| 85B | 8-[(1R)-1-[(2-Benzylsulfanyl-6-chloro- 3-pyridyl)oxy]ethyl]-3,6-dimethyl-2-(2- methyltriazol-4-yl)chromen-4-one | 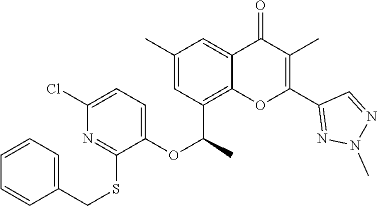 [SMILES: Cc1cc([C@@H](C)Oc2ccc(Cl)nc2SCc2ccccc2)c2oc(-c3cnn(C)n3)c(C)c(=O)c2c1] | 533 [M + H]⁺ |
| 86B | 8-[(1R)-1-[(2-Benzylsulfanyl-6-chloro- 3-pyridyl)oxy]ethyl]-3,6-dimethyl-2- pyrimidin-5-yl-chromen-4-one | 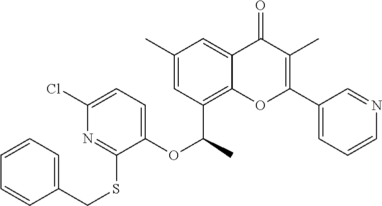 [SMILES: Cc1cc([C@@H](C)Oc2ccc(Cl)nc2SCc2ccccc2)c2oc(-c3cccnc3)c(C)c(=O)c2c1] | 530 [M + H]⁺ |
| 87B | 8-[(1R)-1-[(2-Benzylsulfanyl-6-chloro- 3-pyridyl)oxy]ethyl]-3,6-dimethyl-2-(3- pyridyl)chromen-4-one | 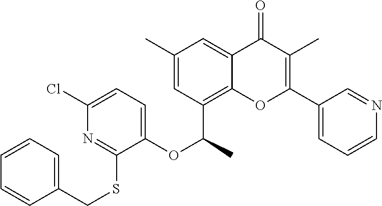 [SMILES: Cc1cc([C@@H](C)Oc2ccc(Cl)nc2SCc2ccccc2)c2oc(-c3cccnc3)c(C)c(=O)c2c1] | 529 [M + H]⁺ |
| 88B | 8-[(1R)-1-[(2-Benzylsulfanyl-6-chloro- 3-pyridyl)oxy]ethyl]-3,6-dimethyl-2-(2- methylpyrimidin-5-yl)chromen-4-one | 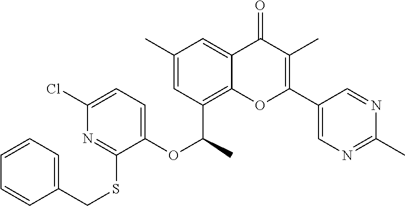 [SMILES: Cc1cc([C@@H](C)Oc2ccc(Cl)nc2SCc2ccccc2)c2oc(-c3cnc(C)nc3)c(C)c(=O)c2c1] | 544 [M + H]⁺ |
| 89B | 8-[(1R)-1-[(2-Benzylsulfanyl-6-chloro- 3-pyridyl)oxy]ethyl]-2-(2- cyclopropylpyrimidin-5-yl)-3,6- dimethyl-chromen-4-one | 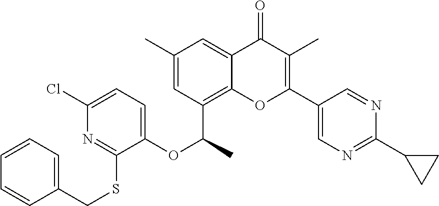 [SMILES: Cc1cc([C@@H](C)Oc2ccc(Cl)nc2SCc2ccccc2)c2oc(-c3cnc(C4CC4)nc3)c(C)c(=O)c2c1] | 570 [M + H]⁺ |
| 90B | 8-[(1R)-1-[(2-Benzylsulfanyl-6-chloro- 3-pyridyl)oxy]ethyl]-2-[1-(2-hydroxy- 2-methyl-propyl)pyrazol-4-yl]-3,6- dimethyl-chromen-4-one | 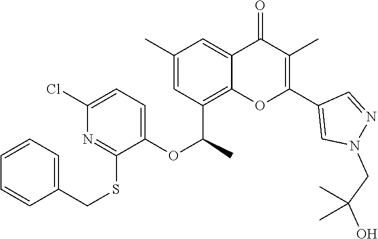 [SMILES: Cc1cc([C@@H](C)Oc2ccc(Cl)nc2SCc2ccccc2)c2oc(-c3cnn(CC(C)(C)O)c3)c(C)c(=O)c2c1] | 590 [M + H]⁺ |
| 91B | 8-[(1R)-1-[(2-Benzylsulfanyl-6-chloro- 3-pyridyl)oxy]ethyl]-2-(6,7-dihydro- 4H-pyrazolo[5,1-c][1,4]oxazin-3-yl)- 3,6-dimethyl-chromen-4-one | 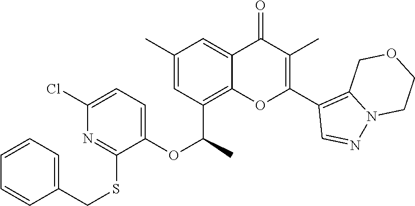 [SMILES: Cc1cc([C@@H](C)Oc2ccc(Cl)nc2SCc2ccccc2)c2oc(-c3cnn4c3COCC4)c(C)c(=O)c2c1] | 574 [M + H]⁺ |
| 92B | 8-[(1R)-1-[(2-Benzylsulfanyl-6-chloro- 3-pyridyl)oxy]ethyl]-2-[1-[(2R)-2- hydroxypropyl]pyrazol-4-yl]-3,6- dimethyl-chromen-4-one | 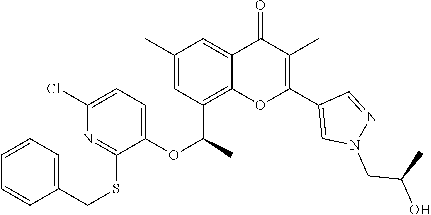 [SMILES: Cc1cc([C@@H](C)Oc2ccc(Cl)nc2SCc2ccccc2)c2oc(-c3cnn(C[C@@H](C)O)c3)c(C)c(=O)c2c1] | 576 [M + H]⁺ |
| 93B | 8-[(1R)-1-[(2-Benzylsulfanyl-6-chloro- 3-pyridyl)oxy]ethyl]-3,6-dimethyl-2-(2- methylpyrazolo[4,3-b]pyridin-5- yl)chromen-4-one | 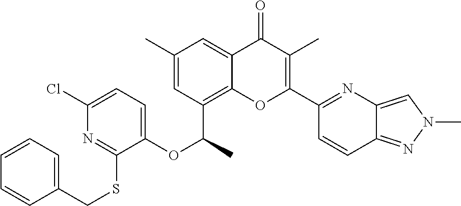 [SMILES: Cc1cc([C@@H](C)Oc2ccc(Cl)nc2SCc2ccccc2)c2oc(-c3ccc4nn(C)cc4n3)c(C)c(=O)c2c1] | 583 [M + H]⁺ |
| 94B | 8-[(1R)-1-[(2-Benzylsulfanyl-6-chloro- 3-pyridyl)oxy]ethyl]-2-(2,3-dihydro- [1,4]dioxino[2,3-b]pyridin-7-yl)-3,6- dimethyl-chromen-4-one | 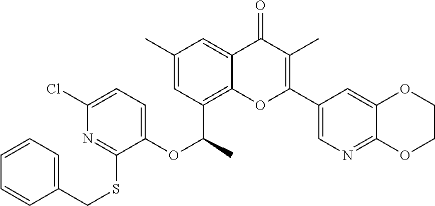 [SMILES: Cc1cc([C@@H](C)Oc2ccc(Cl)nc2SCc2ccccc2)c2oc(-c3cnc4c(c3)OCCO4)c(C)c(=O)c2c1] | 587 [M + H]⁺ |
| 95B | 8-[(1R)-1-[(2-Benzylsulfanyl-6-chloro- 3-pyridyl)oxy]ethyl]-2-[1-[(2S)-2-[tert- butyl(dimethyl)silyl]oxypropyl]pyrazol- 4-yl]-3,6-dimethyl-chromen-4-one | 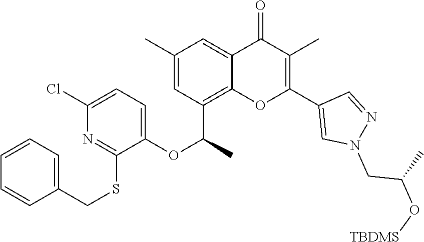 | 690 [M + H]⁺ |
| 96B | 8-[(1R)-1-[(2-Benzylsulfanyl-6-chloro- 3-pyridyl)oxy]ethyl]-2-[1-[2-[tert- butyl(dimethyl)silyl]oxyethyl]pyrazol- 4-yl]-3,6-dimethyl-chromen-4-one | 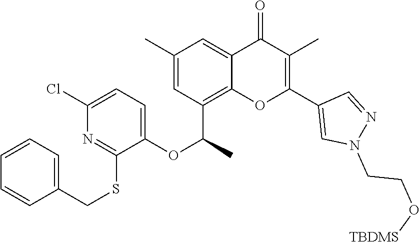 | 676 [M + H]⁺ |
| 97B | 8-[(1R)-1-[(2-Benzylsulfanyl-6-chloro- 3-pyridyl)oxy]ethyl]-2-cyclopropyl-3,6- dimethyl-chromen-4-one | 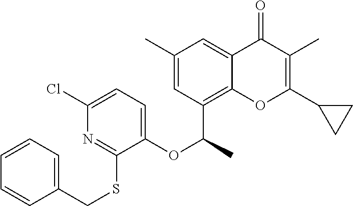 [SMILES: Cc1cc([C@@H](C)Oc2ccc(Cl)nc2SCc2ccccc2)c2oc(C3CC3)c(C)c(=O)c2c1] | 492 [M + H]⁺ |
| 114C | 8-[(1R)-1-[(2-Benzylsulfanyl-6-chloro- 3-pyridyl)oxy]ethyl]-3,6-dimethyl-2- (1H-pyrazol-5-yl)chromen-4-one | 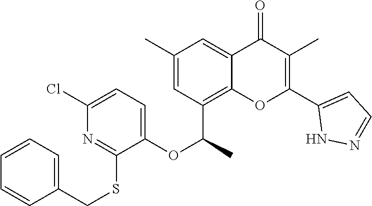 [SMILES: Cc1cc([C@@H](C)Oc2ccc(Cl)nc2SCc2ccccc2)c2oc(-c3ccn[nH]3)c(C)c(=O)c2c1] | 518 [M + H]⁺ |
| 115C | ethyl 2-[8-[(1R)-1-[(2-Benzylsulfanyl- 6-chloro-3-pyridyl)oxy]ethyl]-3,6- dimethyl-4-oxo-chromen-2- yl]cyclopropanecarboxylate | 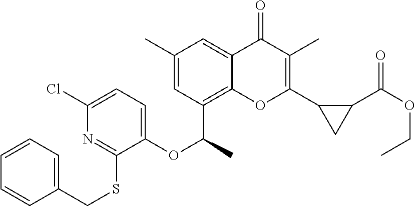 [SMILES: CCOC(=O)C1CC1c1oc2c([C@@H](C)Oc3ccc(Cl)nc3SCc3ccccc3)cc(C)cc2c(=O)c1C] | 564 [M + H]⁺ |

### Intermediate 45A: (2,3,4,5,6-Pentafluorophenyl) 6-chloro-3-[(1R)-1-(3,6-dimethyl-4-oxo-2-phenyl-chromen-8-yl)ethoxy]pyridine-2-sulfonate

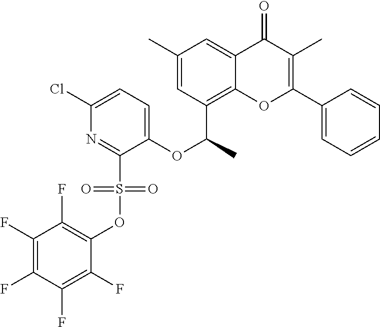 [SMILES: Cc1cc([C@@H](C)Oc2ccc(Cl)nc2S(=O)(=O)Oc2c(F)c(F)c(F)c(F)c2F)c2oc(-c3ccccc3)c(C)c(=O)c2c1]

A solution of 8+[(R)-1-[(2-benzylsulfanyl-6-chloro-3-pyridyl)oxy]ethyl]-3,6-dimethyl-2-phenyl-chromen-4-one (100 mg, 0.19 mmol) in AcOH/water (3:1; 2 mL) was treated with N-chlorosuccinimide (101 mg, 0.76 mmol) at 0° C. The cooling bath was removed and the reaction stirred at rt for 2 h. The reaction was re-cooled to 0° C. and treated with pentafluorophenol (349 mg, 1.89 mmol) and DIPEA (245 mg, 1.89 mmol). The cooling bath was removed and the reaction stirred at rt for 2 h. The reaction was diluted with EtOAc and washed with saturated aqueous NaHCO₃. The aqueous layer was re-extracted with EtOAc (2×). The organics were combined, dried over Na₂SO₄, filtered, and concentrated under reduced pressure. The residue was purified by silica gel chromatography eluted with 000 to 100% EtOAc in heptane to give the title compound (85 mg, 690m) as a white solid. ES-MS m/z 652 (M+H).

The following compounds in Table 18 were made in a similar way as described for (2,3,4,5,6-pentafluorophenyl) 6-chloro-3-[(1R)-1-(3,6-dimethyl-4-oxo-2-phenyl-chromen-8-yl)ethoxy]pyridine-2-sulfonate. Various methods were used to purify the compounds, which would be apparent to one skilled in the art.

TABLE 18

| | | | MS ES+ |
|---|---|---|---|
| Interm # | Chemical Name | Structure | m/z |
| 46A | (2,3,4,5,6-Pentafluorophenyl) 6-chloro-3-[(1R)-1-[3,6- dimethyl-2-(1-methylpyrazol- 4-yl)-4-oxo-chromen-8- yl]ethoxy]pyridine-2- sulfonate | 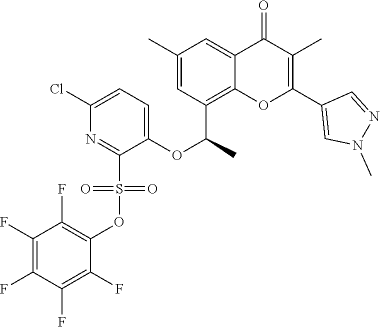 [SMILES: Cc1cc([C@@H](C)Oc2ccc(Cl)nc2S(=O)(=O)Oc2c(F)c(F)c(F)c(F)c2F)c2oc(-c3cnn(C)c3)c(C)c(=O)c2c1] | 656 [M + H]⁺ |
| 47A | (2,3,4,5,6-Pentafluorophenyl) 6-chloro-3-[(1R)-1-[2- ([1,3]dioxolo[4,5-b]pyridin- 6-yl)-3,6-dimethyl-4-oxo- chromen-8- yl]ethoxy]pyridine-2- sulfonate | 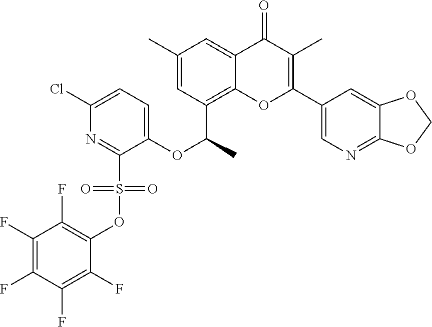 [SMILES: Cc1cc([C@@H](C)Oc2ccc(Cl)nc2S(=O)(=O)Oc2c(F)c(F)c(F)c(F)c2F)c2oc(-c3cnc4c(c3)OCO4)c(C)c(=O)c2c1] | 697 [M + H]⁺ |
| 48A | (2,3,4,5,6-Pentafluorophenyl) 6-chloro-3-[(1R)-1-[3,6- dimethyl-2-(2-methylindazol- 5-yl)-4-oxo-chromen-8- yl]ethoxy]pyridine-2- sulfonate | 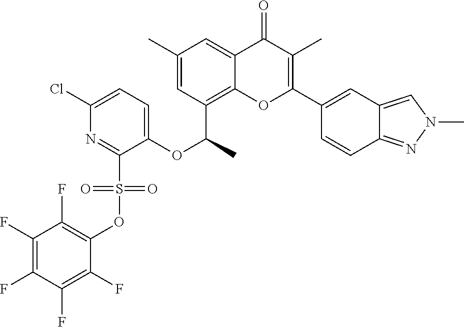 [SMILES: Cc1cc([C@@H](C)Oc2ccc(Cl)nc2S(=O)(=O)Oc2c(F)c(F)c(F)c(F)c2F)c2oc(-c3ccc4nn(C)cc4c3)c(C)c(=O)c2c1] | 706 [M + H]⁺ |
| 98B | (2,3,4,5,6-Pentafluorophenyl) 6-chloro-3-[(1R)-1-[3,6- dimethyl-2-(2-methyltriazol- 4-yl)-4-oxo-chromen-8- yl]ethoxy]pyridine-2- sulfonate | 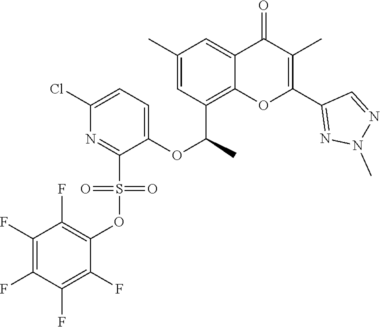 [SMILES: Cc1cc([C@@H](C)Oc2ccc(Cl)nc2S(=O)(=O)Oc2c(F)c(F)c(F)c(F)c2F)c2oc(-c3cnn(C)n3)c(C)c(=O)c2c1] | 657 [M + H]⁺ |
| 99B | (2,3,4,5,6-Pentafluorophenyl) 6-chloro-3-[(1R)-1-(3,6- dimethyl-4-oxo-2-pyrimidin- 5-yl-chromen-8- yl)ethoxy]pyridine-2- sulfonate | 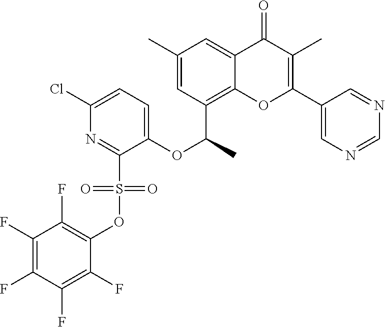 [SMILES: Cc1cc([C@@H](C)Oc2ccc(Cl)nc2S(=O)(=O)Oc2c(F)c(F)c(F)c(F)c2F)c2oc(-c3cncnc3)c(C)c(=O)c2c1] | 654 [M + H]⁺ |
| 100B | (2,3,4,5,6-Pentafluorophenyl) 6-chloro-3-[(1R)-1-[3,6- dimethyl-4-oxo-2-(3- pyridyl)chromen-8- yl]ethoxy]pyridine-2- sulfonate | 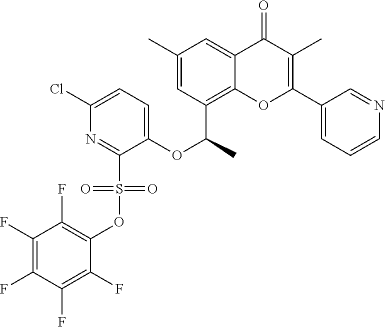 [SMILES: Cc1cc([C@@H](C)Oc2ccc(Cl)nc2S(=O)(=O)Oc2c(F)c(F)c(F)c(F)c2F)c2oc(-c3cccnc3)c(C)c(=O)c2c1] | 653 [M + H]⁺ |
| 101B | (2,3,4,5,6-Pentafluorophenyl) 6-chloro-3-[(1R)-1-[3,6- dimethyl-2-(2- methylpyrimidin-5-yl)-4-oxo- chromen-8- yl]ethoxy]pyridine-2- sulfonate | 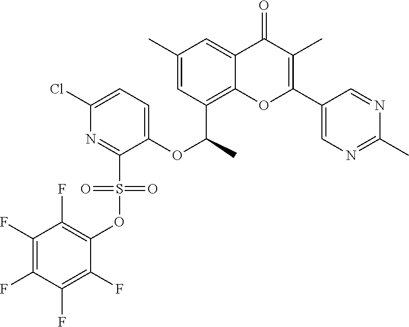 [SMILES: Cc1cc([C@@H](C)Oc2ccc(Cl)nc2S(=O)(=O)Oc2c(F)c(F)c(F)c(F)c2F)c2oc(-c3cnc(C)nc3)c(C)c(=O)c2c1] | 668 [M + H]⁺ |
| 102B | (2,3,4,5,6-Pentafluorophenyl) 6-chloro-3-[(1R)-1-[2-(2- cyclopropylpyrimidin-5-yl)- 3,6-dimethyl-4-oxo-chromen- 8-yl]ethoxy]pyridine-2- sulfonate | 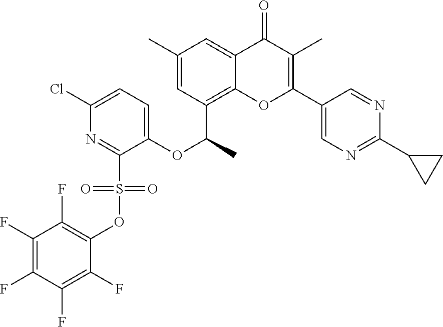 [SMILES: Cc1cc([C@@H](C)Oc2ccc(Cl)nc2S(=O)(=O)Oc2c(F)c(F)c(F)c(F)c2F)c2oc(-c3cnc(C4CC4)nc3)c(C)c(=O)c2c1] | 694 [M + H]⁺ |
| 103B | (2,3,4,5,6-Pentafluorophenyl) 6-chloro-3-[(1R)-1-[2-[1-(2- hydroxy-2-methyl- propyl)pyrazol-4-yl]-3,6- dimethyl-4-oxo-chromen-8- yl]ethoxy]pyridine-2- sulfonate | 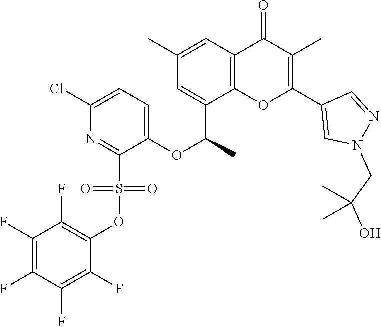 [SMILES: Cc1cc([C@@H](C)Oc2ccc(Cl)nc2S(=O)(=O)Oc2c(F)c(F)c(F)c(F)c2F)c2oc(-c3cnn(CC(C)(C)O)c3)c(C)c(=O)c2c1] | 714 [M + H]⁺ |
| 104B | (2,3,4,5,6-Pentafluorophenyl) 6-chloro-3-[(1R)-1-[2-(6,7- dihydro-4H-pyrazolo[5,1- c][1,4]oxazin-3-yl)-3,6- dimethyl-4-oxo-chromen-8- yl]ethoxy]pyridine-2- sulfonate | 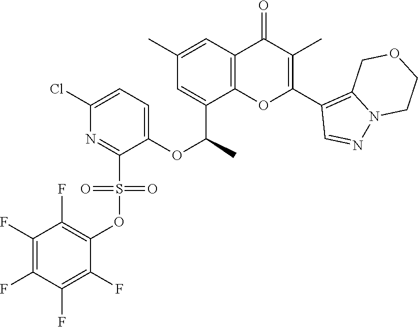 [SMILES: Cc1cc([C@@H](C)Oc2ccc(Cl)nc2S(=O)(=O)Oc2c(F)c(F)c(F)c(F)c2F)c2oc(-c3cnn4c3COCC4)c(C)c(=O)c2c1] | 698 [M + H]⁺ |
| 105B | (2,3,4,5,6-Pentafluorophenyl) 6-chloro-3-[(1R)-1-[2-[1- [(2R)-2- hydroxypropyl]pyrazol-4-yl]- 3,6-dimethyl-4-oxo-chromen- 8-yl]ethoxy]pyridine-2- sulfonate | 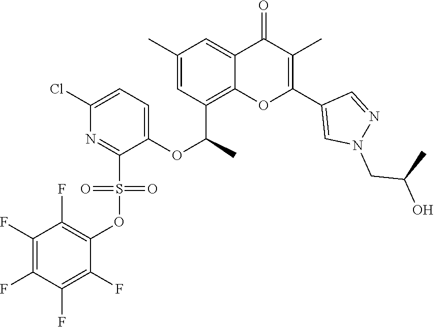 [SMILES: Cc1cc([C@@H](C)Oc2ccc(Cl)nc2S(=O)(=O)Oc2c(F)c(F)c(F)c(F)c2F)c2oc(-c3cnn(C[C@@H](C)O)c3)c(C)c(=O)c2c1] | 700 [M + H]⁺ |
| 106B | (2,3,4,5,6-Pentafluorophenyl) 6-chloro-3-[(1R)-1-[3,6- dimethyl-2-(2- methylpyrazolo[4,3- b]pyridin-5-yl)-4-oxo- chromen-8- yl]ethoxy]pyridine-2- sulfonate | 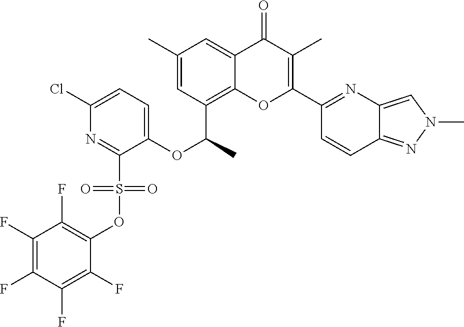 [SMILES: Cc1cc([C@@H](C)Oc2ccc(Cl)nc2S(=O)(=O)Oc2c(F)c(F)c(F)c(F)c2F)c2oc(-c3ccc4nn(C)cc4n3)c(C)c(=O)c2c1] | 707 [M + H]⁺ |
| 107B | (2,3,4,5,6-Pentafluorophenyl) 6-chloro-3-[(1R)-1-[2-(2,3- dihydro-[1,4]dioxino[2,3- b]pyridin-7-yl)-3,6-dimethyl- 4-oxo-chromen-8- yl]ethoxy]pyridine-2- sulfonate | 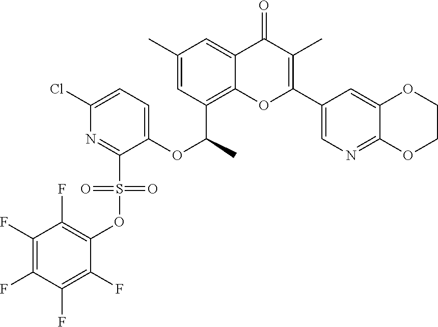 [SMILES: Cc1cc([C@@H](C)Oc2ccc(Cl)nc2S(=O)(=O)Oc2c(F)c(F)c(F)c(F)c2F)c2oc(-c3cnc4c(c3)OCCO4)c(C)c(=O)c2c1] | 711 [M + H]⁺ |
| 108B | (2,3,4,5,6-Pentafluorophenyl) 6-chloro-3-[(1R)-1-[2-[1- [(2S)-2- hydroxypropyl]pyrazol-4-yl]- 3,6-dimethyl-4-oxo-chromen- 8-yl]ethoxy]pyridine-2- sulfonate | 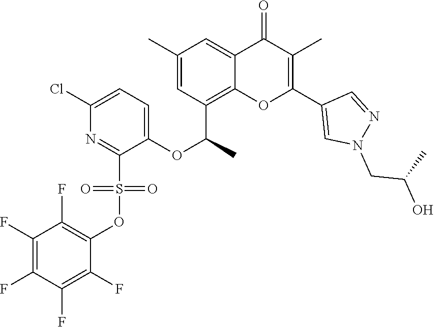 [SMILES: Cc1cc([C@@H](C)Oc2ccc(Cl)nc2S(=O)(=O)Oc2c(F)c(F)c(F)c(F)c2F)c2oc(-c3cnn(C[C@H](C)O)c3)c(C)c(=O)c2c1] | 700 [M + H]⁺ |
| 109B | (2,3,4,5,6-Pentafluorophenyl) 6-chloro-3-[(1R)-1-[2-[1-(2- hydroxyethyl)pyrazol-4-yl]- 3,6-dimethyl-4-oxo-chromen- (8-yl]ethoxy]pyridine-2- sulfonate | 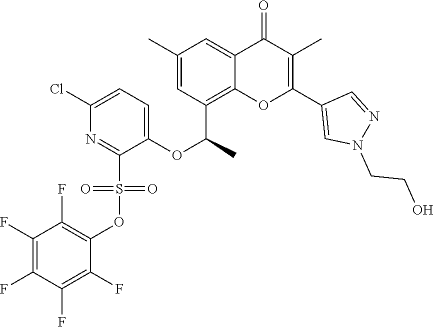 [SMILES: Cc1cc([C@@H](C)Oc2ccc(Cl)nc2S(=O)(=O)Oc2c(F)c(F)c(F)c(F)c2F)c2oc(-c3cnn(CCO)c3)c(C)c(=O)c2c1] | 686 [M + H]⁺ |
| 110B | (2,3,4,5,6-Pentafluorophenyl) 6-chloro-3-[(1R)-1-(2- cyclopropyl-3,6-dimethyl-4- oxo-chromen-8- yl)ethoxy]pyridine-2- sulfonate | 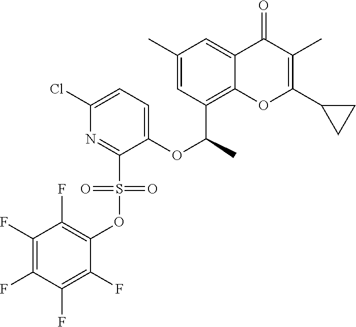 [SMILES: Cc1cc([C@@H](C)Oc2ccc(Cl)nc2S(=O)(=O)Oc2c(F)c(F)c(F)c(F)c2F)c2oc(C3CC3)c(C)c(=O)c2c1] | 616 [M + H]⁺ |
| 116C | ethyl 2-[8-[(1R)-1-[6- Chloro-2-(2,3,4,5,6- pentafluorophenoxy)sulfonyl- 3-pyridyl]oxylethyl]-3,6- dimethyl-4-oxo-chromen-2- yl]cyclopropanecarboxylate | 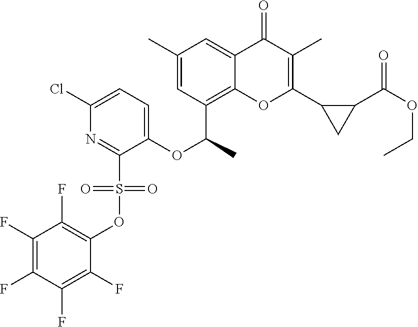 [SMILES: CCOC(=O)C1CC1c1oc2c([C@@H](C)Oc3ccc(Cl)nc3S(=O)(=O)Oc3c(F)c(F)c(F)c(F)c3F)cc(C)cc2c(=O)c1C] | 688 [M + H]⁺ |

### Intermediate 117C: ethyl 2-[8-[(1R)-1-[(6-Chloro-2-sulfamoyl-3-pyridyl)oxy]ethyl]-3,6-dimethyl-4-oxo-chromen-2-yl]cyclopropanecarboxylate

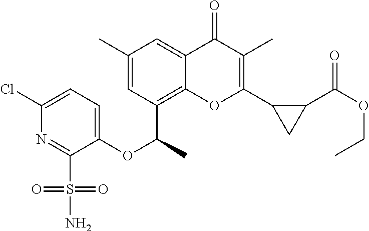 [SMILES: CCOC(=O)C1CC1c1oc2c([C@@H](C)Oc3ccc(Cl)nc3S(N)(=O)=O)cc(C)cc2c(=O)c1C]

A solution of ethyl 2-[8-[(1R)-1-[[6-chloro-2-(2,3,4,5,6-pentafluorophenoxy)sulfonyl-3-pyridyl]oxy]ethyl]-3,6-dimethyl-4-oxo-chromen-2-yl]cyclopropanecarboxylate (1.13 g, 1.64 mmol) in THE (16 mL) was treated with aqueous ammonia (28%, 1.13 mL, 8.21 mmol) and then stirred at 60° C. for 30 min. The reaction was concentrated under reduced pressure and the residue was purified by reversed phase chromatography eluted with a gradient of 0 to 100% ACN in H₂O+0.1% formic acid. Fractions containing the desired product were combined, washed with saturated aqueous sodium chloride, and extracted with IPA:CHCl₃ (1:3). The combined extracts were dried over MgSO₄, filtered, and concentrated under reduced pressure. The material was recrystallized in DCM/MeOH/hexanes to give the title compound (0.220 g, 26%) as a white solid. MS ES+ m/z 521 [M+H]⁺.

### Intermediate 22: 6-Chloro-3-[(1R)-1-(3,6-dimethyl-4-oxo-2-phenyl-chromen-8-yl)ethoxy]pyridine-2-carboxylic acid

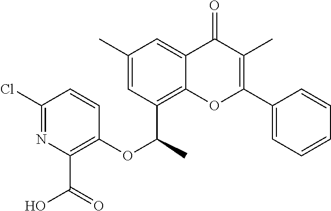 [SMILES: Cc1cc([C@@H](C)Oc2ccc(Cl)nc2C(=O)O)c2oc(-c3ccccc3)c(C)c(=O)c2c1]

To a solution of methyl 6-chloro-3-[(1R)-1-(3,6-dimethyl-4-oxo-2-phenyl-chromen-8-yl)ethoxy]pyridine-2-carboxylate (0.624 g, 50% purity, 0.673 mmol) in THE (9 mL) and water (3 mL) was added lithium hydroxide (0.0644 g, 2.69 mmol). The reaction was heated at 50° C. for 2 h and diluted with DCM and saturated aqueous ammonium chloride. The layers were separated. The aqueous layer was extracted twice with IPA:CHCl₃ (1:3). The combined organic extracts were dried over MgSO₄, filtered, and concentrated under reduced pressure to give the title compound (0.158 g, 52%). MS ES+ m/z 450 [M+H]⁺.

The following compounds in Table 19 were made in a similar way as described for 6-chloro-3-[(1R)-1-(3,6-dimethyl-4-oxo-2-phenyl-chromen-8-yl)ethoxy]pyridine-2-carboxylic acid. Various methods were used to purify the compounds, which would be apparent to one skilled in the art.

TABLE 19

| | | | MS ES+ |
|---|---|---|---|
| Interm # | Chemical Name | Structure | m/z |
| 49A | 6-[(1R)-1-[3,6-Dimethyl- 2-(2-methylindazol-5-yl)- 4-oxo-chromen-8- yl]ethoxy]-2,3-difluoro- benzoic acid | 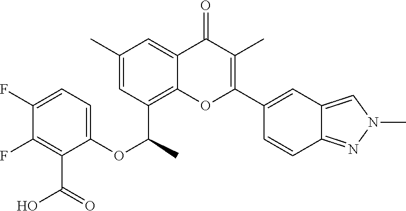 [SMILES: Cc1cc([C@@H](C)Oc2ccc(F)c(F)c2C(=O)O)c2oc(-c3ccc4nn(C)cc4c3)c(C)c(=O)c2c1] | 505 [M + H]⁺ |
| 111B | 6-Chloro-3-[(1R)-1-(2- ethylsulfanyl-3,6- dimethyl-4-oxo-chromen- 8-yl)ethoxy]pyridine-2- carboxylic acid | 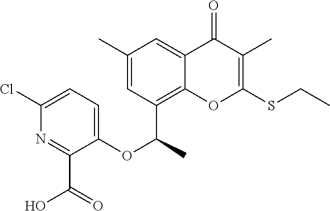 [SMILES: CCSc1oc2c([C@@H](C)Oc3ccc(Cl)nc3C(=O)O)cc(C)cc2c(=O)c1C] | 434 [M + H]⁺ |
| 112B | 6-[(1R)-1-[3,6-Dimethyl- 2-(1-methylpyrazol-4-yl)- 4-oxo-chromen-8- yl]ethoxy]-2,3-difluoro- benzoic acid | 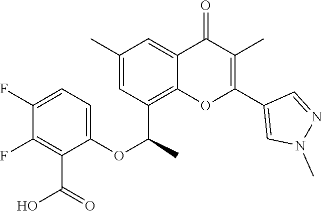 [SMILES: Cc1cc([C@@H](C)Oc2ccc(F)c(F)c2C(=O)O)c2oc(-c3cnn(C)c3)c(C)c(=O)c2c1] | 455 [M + H]⁺ |
| 113B | 3-[(1R)-1-[3,6-Dimethyl- 2-(2-methylindazol-5-yl)- 4-oxo-chromen-8- yl]ethoxy]pyridine-2- carboxylic acid | 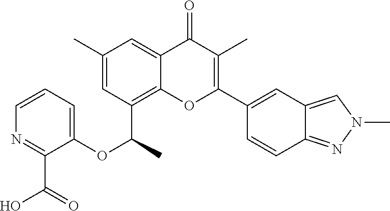 [SMILES: Cc1cc([C@@H](C)Oc2cccnc2C(=O)O)c2oc(-c3ccc4nn(C)cc4c3)c(C)c(=O)c2c1] | 470 [M + H]⁺ |
| 114B | 6-Chloro-3-[(1R)-1-[3,6- dimethyl-2-(1- methylpyrazol-4-yl)-4- oxo-chromen-8- yl]ethoxy]pyridine-2- carboxylic acid | 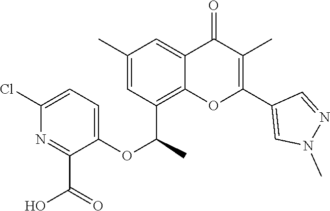 [SMILES: Cc1cc([C@@H](C)Oc2ccc(Cl)nc2C(=O)O)c2oc(-c3cnn(C)c3)c(C)c(=O)c2c1] | 454 [M + H]⁺ |
| 115B | 6-Chloro-3-[(1R)-1-[3,6- dimethyl-4-oxo-2-(2- pyridyl)chromen-8- yl]ethoxy]pyridine-2- carboxylic acid | 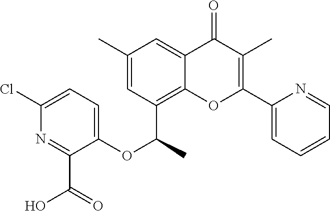 [SMILES: Cc1cc([C@@H](C)Oc2ccc(Cl)nc2C(=O)O)c2oc(-c3ccccn3)c(C)c(=O)c2c1] | 451 [M + H]⁺ |
| 116B | 6-Chloro-3-[(1R)-1-[2-(5- fluoro-3-pyridyl)-3,6- dimethyl-4-oxo-chromen- 8-yl]ethoxy]pyridine-2- carboxylic acid | 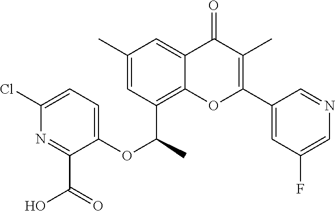 [SMILES: Cc1cc([C@@H](C)Oc2ccc(Cl)nc2C(=O)O)c2oc(-c3cncc(F)c3)c(C)c(=O)c2c1] | 469 [M + H]⁺ |
| 118C | 2-[8-[(1R)-1-[(6-Chloro- 2-sulfamoyl-3- pyridyl)oxy]ethyl]-3,6- dimethyl-4-oxo-chromen- 2- yl]cyclopropanecarboxylic acid | 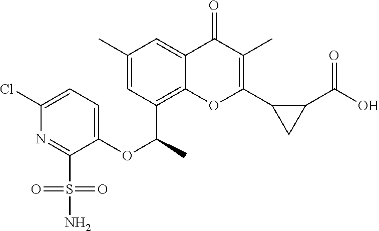 [SMILES: Cc1cc([C@@H](C)Oc2ccc(Cl)nc2S(N)(=O)=O)c2oc(C3CC3C(=O)O)c(C)c(=O)c2c1] | 493 [M + H]⁺ |
| 119C | 2-[1-[8-[(1R)-1-[(6- Chloro-2-sulfamoyl-3- pyridyl)oxyJethyl]-3,6- dimethyl-4-oxo-chromen- 2-yl]cyclopropyl]acetic acid | 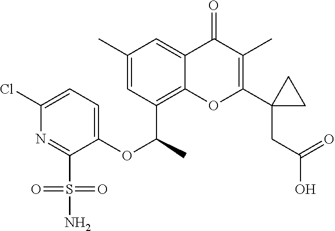 [SMILES: Cc1cc([C@@H](C)Oc2ccc(Cl)nc2S(N)(=O)=O)c2oc(C3(CC(=O)O)CC3)c(C)c(=O)c2c1] | 507 [M + H]⁺ |

### Intermediate 117B: 6-Chloro-3-[(1R)-1-(2-ethylsulfanyl-3,6-dimethyl-4-oxo-chromen-8-yl)ethoxy]pyridine-2-carboxamide

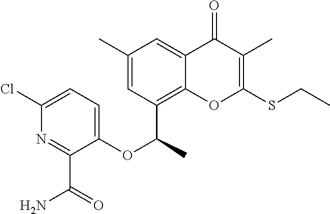 [SMILES: CCSc1oc2c([C@@H](C)Oc3ccc(Cl)nc3C(N)=O)cc(C)cc2c(=O)c1C]

Dissolved 6-chloro-3-[(1R)-1-(2-ethylsulfanyl-3,6-dimethyl-4-oxo-chromen-8-yl)ethoxy]pyridine-2-carboxylic acid (0.8 g, 2 mmol) in DMF (3 mL) and treated with HATU (0.9 g, 2 mmol) and DIPEA (1 mL, 6 mmol). The reaction was stirred at rt for 10 min and then added ammonia (0.4 M in 1,4-dioxane, 20 mL, 7 mmol). The reaction was stirred at rt for 4 h. The reaction was concentrated under reduced pressure, the residue dissolved in 2 mL of DMF, and purified by reversed phase chromatography eluted with 30% to 55% ACN in 10 mM aqueous NH₄HCO₃ with 5% MeOH to give the title compound (0.3 g, 40%) as a white solid. MS ES− m/z 431 [M−H]⁻.

### Intermediate 120C: 6-Chloro-3-[(1R)-1-(2-ethylsulfanyl-3,6-dimethyl-4-oxo-chromen-8-yl)ethoxy]-N-methoxy-pyridine-2-carboxamide

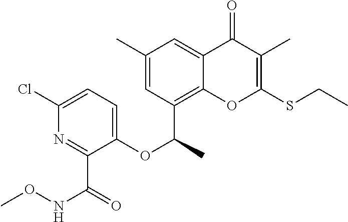 [SMILES: CCSc1oc2c([C@@H](C)Oc3ccc(Cl)nc3C(=O)NOC)cc(C)cc2c(=O)c1C]

Combined 6-chloro-3-[(1R)-1-(2-ethylsulfanyl-3,6-dimethyl-4-oxo-chromen-8-yl)ethoxy]pyridine-2-carboxylic acid (50%; 2.69 g, 3.10 mmol), diisopropylethylamine (5.40 mL, 31.0 mmol), O-methylhydroxylammonium chloride (0.647 g, 7.75 mmol), 4-dimethylaminopyridine (0.038 g, 0.310 mmol) and propylphosphonic anhydride (50 wt % in DMF, 4.75 mL, 7.75 mmol) in DMF (7.5 mL). The reaction mixture was stirred at 25° C. for 40 min, diluted with water (50 mL), and extracted with EtOAc (3×50 mL). The combined organic layers were washed with saturated aqueous sodium chloride (2×), dried over sodium sulfate, filtered, and concentrated under reduced pressure. The residue was purified by silica gel chromatography eluted with 0% to 75% EtOAc in heptane to give the title compound (1.38 g, 91%) as a white solid. MS ES− m/z 461 [M−H]⁻.

The following compounds in Table 20 were made in a similar way as described for 8-[(1S)-1-hydroxyethyl]-3,6-dimethyl-2-(2-pyridyl)chromen-4-one. Various methods were used to purify the compounds, which would be apparent to one skilled in the art.

TABLE 20

| | | | MS ES+ |
|---|---|---|---|
| Interm # | Chemical Name | Structure | m/z |
| 121C | 3-[(1R)-1-[2-[2-[2-[tert- Butyl(dimethyl)silyl] oxyethyl]indazol-5-yl]-3,6- dimethyl-4-oxo-chromen- 8-yl]ethoxy]-6-chloro-N- methoxy-pyridine-2- carboxamide | 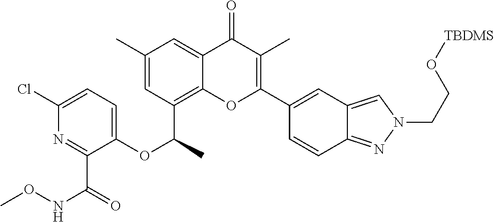 | 675 [M − H]⁻ |
| 122C | 3-[(1R)-1-[2-[2-[2-[tert- Butyl(dimethyl)silyl] oxyethyl]indazol-5-yl]-3,6- dimethyl-4-oxo-chromen- 8-yl]ethoxy]-6-chloro- pyridine-2-carboxamide | 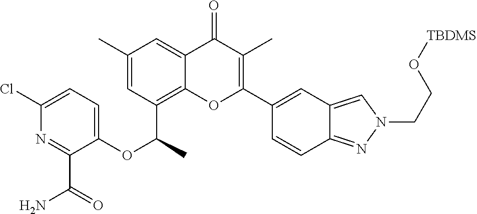 | 645 [M − H]⁻ |
| 123C | 6-Chloro-3-[(1R)-1-[3,6- dimethyl-4-oxo-2-[2-[(1- tetrahydropyran-2- yloxycyclopropyl)methyl] indazol-5-yl]chromen-8- yl]ethoxy]pyridine-2- carboxamide | 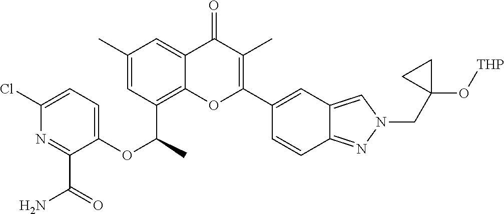 | 641 [M − H]⁻ |
| 124C | 3-[(1R)-1-[2-[2-[2-[tert- Butyl(dimethyl)silyl] oxyethyl] imidazo[1,2- a]pyridin-6-yl]-3,6- dimethyl-4-oxo-chromen- 8-yl]ethoxy]-6-chloro- pyridine-2-carboxamide | 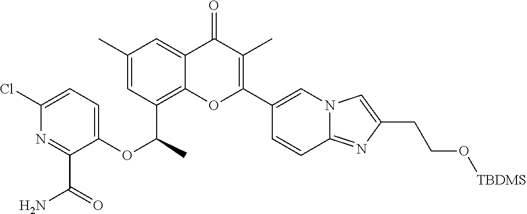 | 647 [M + H]⁺ |
| 125C | 3-[(1R)-1-[2-[2-[2-[tert- Butyl(dimethyl)silyl] oxyethyl]-1,3-benzothiazol-6- yl]-3,6-dimethyl-4-oxo- chromen-8-yl]ethoxy]-6- chloro-pyridine-2- carboxamide | 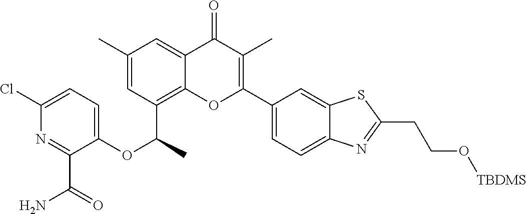 | 662 [M − H]⁻ |
| 126C | 3-[(1R)-1-[2-[2-[3-[tert- Butyl (dimethyl)silyl]oxycyclobutyl] indazol-5-yl]-3,6- dimethyl-4-oxo-chromen- 8-yl]ethoxy]-6-chloro- pyridine-2-carboxamide | 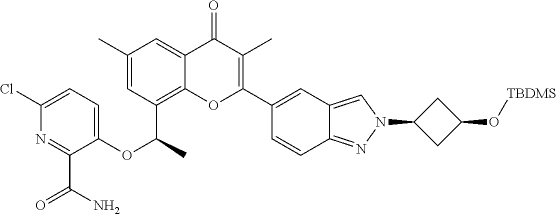 | 673 [M + H]⁺ |
| 127C | 3-[(1R)-1-[2-[2-[3-[tert- Butyl (dimethyl)silyl]oxycyclobutyl] indazol-5-yl]-3,6- dimethyl-4-oxo-chromen- 8-yl]ethoxy]-6-chloro- pyridine-2-carboxamide | 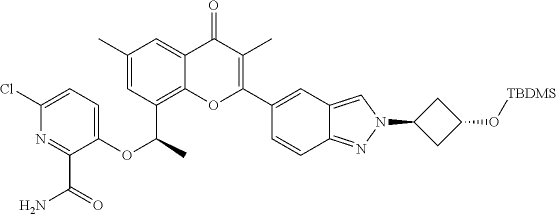 | 673 [M + H]⁺ |

### Example 1: 8-[(1R)-1-[(6-Chloro-2-methyl-3-pyridyl)oxy]ethyl]-3,6-dimethyl-2-phenyl-chromen-4-one

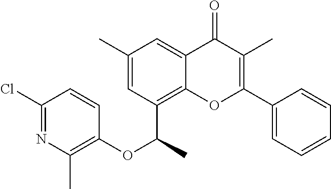 [SMILES: Cc1cc([C@@H](C)Oc2ccc(Cl)nc2C)c2oc(-c3ccccc3)c(C)c(=O)c2c1]

Combined 8-[(1R)-1-hydroxyethyl]-3,6-dimethyl-2-phenyl-chromen-4-one (0.100 g, 0.340 mmol), 6-chloro-3-iodo-2-methyl-pyridine (0.0861 g, 0.340 mmol), tripotassium phosphate (0.144 g, 0.679 mmol), copper(I) iodide (6.47 mg, 0.034 mmol), and 4-pyrrolidinopyridine (0.0604 g, 0.408 mmol) in toluene (3 mL). The mixture was sparged with nitrogen (direct line) for 3 min. The reaction was heated at reflux for 19 h, filtered, and the filtrate was concentrated under reduced pressure. The residue was purified by reversed phase chromatography eluted with a gradient of 0 to 100% ACN in aqueous NH₄HCO₃ (10 mM, plus 5% MeOH). Fractions containing the desired product were combined, concentrated under reduced pressure, and lyophilized to give the title compound (0.018 g, 12%). MS ES+ m/z 420 [M+H]⁺.

### Example 2: 3-[6-Chloro-3-[(1R)-1-(3,6-dimethyl-4-oxo-2-phenyl-chromen-8-yl)ethoxy]-2-pyridyl]-4H-1,2,4-oxadiazol-5-one

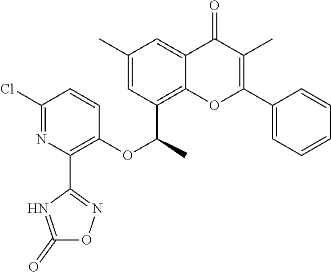

To a solution of 6-chloro-3-[(1R)-1-(3,6-dimethyl-4-oxo-2-phenyl-chromen-8-yl)ethoxy]-N′-hydroxy-pyridine-2-carboxamidine (0.180 g, 0.388 mmol) in 1,4-dioxane (4 mL) was added 1,8-diazabicyclo[5.4.0]undec-7-ene (0.117 mL, 0.776 mmol) and 1,1′-carbonyldiimidazole (0.0944 g, 0.582 mmol). The reaction was sealed, heated under microwave conditions (100° C., 1 h), and concentrated under reduced pressure. The residue was purified by reversed phase chromatography eluted with a gradient of 0 to 100% ACN in H₂O+0.1% formic acid. Fractions containing the desired product were combined, washed with saturated aqueous sodium chloride, and extracted with IPA:CHCl₃ (1:3). The combined extracts were dried over MgSO₄, filtered, and concentrated under reduced pressure to give the title compound (0.0846 g, 45%). MS ES+ m/z 490 [M+H]⁺.

The following compounds in Table 21 were made in a similar way as described for 3-[6-chloro-3-[(1R)-1-(3,6-dimethyl-4-oxo-2-phenyl-chromen-8-yl)ethoxy]-2-pyridyl]-4H-1,2,4-oxadiazol-5-one. Various methods were used to purify the compounds, which would be apparent to one skilled in the art.

TABLE 21

| | | | MS ES+ |
|---|---|---|---|
| Example # | Chemical Name | Structure | m/z |
| 3 | 3-[3-[(1R)-1-(3,6-Dimethyl-4- oxo-2-phenyl-chromen-8- yl)ethoxy]-2-pyridyl]-4H- 1,2,4-oxadiazol-5-one | 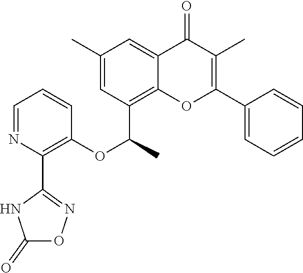 [SMILES: Cc1cc([C@@H](C)Oc2cccnc2-c2noc(=O)[nH]2)c2oc(-c3ccccc3)c(C)c(=O)c2c1] | 456 [M + H]⁺ |
| 4 | 3-[2-[(1R)-1-(3,6-Dimethyl-4- oxo-2-phenyl-chromen-8- yl)ethoxy]phenyl]-4H-1,2,4- oxadiazol-5-one | 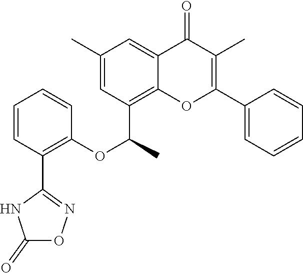 [SMILES: Cc1cc([C@@H](C)Oc2ccccc2-c2noc(=O)[nH]2)c2oc(-c3ccccc3)c(C)c(=O)c2c1] | 455 [M + H]⁺ |
| 5 | 3-[2-[(1R)-1-(3,6-Dimethyl-4- oxo-2-phenyl-chromen-8- yl)ethoxy]-6-fluoro-phenyl]- 4H-1,2,4-oxadiazol-5-one | 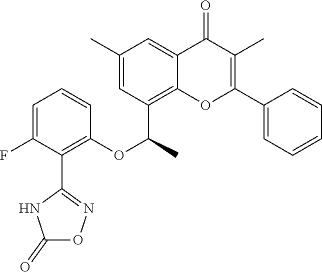 [SMILES: Cc1cc([C@@H](C)Oc2cccc(F)c2-c2noc(=O)[nH]2)c2oc(-c3ccccc3)c(C)c(=O)c2c1] | 473 [M + H]⁺ |
| 1A | 3-[6-Chloro-3-[(1R)-1-[3,6- dimethyl-4-oxo-2-(2- pyridyl)chromen-8-yl]ethoxy]- 2-pyridyl]-4H-1,2,4-oxadiazol- 5-one | 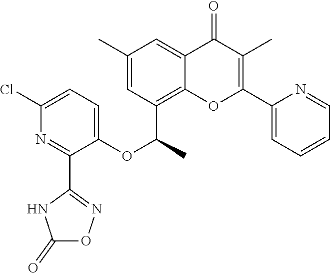 [SMILES: Cc1cc([C@@H](C)Oc2ccc(Cl)nc2-c2noc(=O)[nH]2)c2oc(-c3ccccn3)c(C)c(=O)c2c1] | 491 [M + H]⁺ |
| 2A | 3-[6-Chloro-3-[(1R)-1-[3,6- dimethyl-2-(1-methylpyrazol- 4-yl)-4-oxo-chromen-8- yl]ethoxy]-2-pyridyl]-4H- 1,2,4-oxadiazol-5-one | 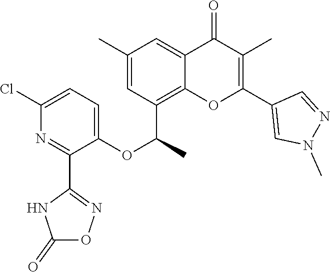 [SMILES: Cc1cc([C@@H](C)Oc2ccc(Cl)nc2-c2noc(=O)[nH]2)c2oc(-c3cnn(C)c3)c(C)c(=O)c2c1] | 494 [M + H]⁺ |
| 3A | 3-[6-Chloro-3-[(1R)-1-[3,6- dimethyl-4-oxo-2-(3- pyridyl)chromen-8-yl]ethoxy]- 2-pyridyl]-4H-1,2,4-oxadiazol- 5-one | 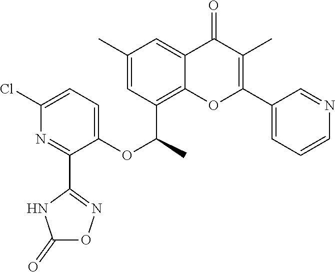 [SMILES: Cc1cc([C@@H](C)Oc2ccc(Cl)nc2-c2noc(=O)[nH]2)c2oc(-c3cccnc3)c(C)c(=O)c2c1] | 491 [M + H]⁺ |
| 4A | 3-[6-Chloro-3-[(1R)-1-[2-(2- fluoro-3-pyridyl)-3,6- dimethyl-4-oxo-chromen-8- yl]ethoxy]-2-pyridyl]-4H- 1,2,4-oxadiazol-5-one | 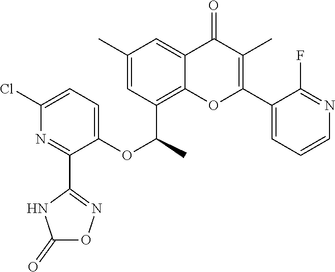 [SMILES: Cc1cc([C@@H](C)Oc2ccc(Cl)nc2-c2noc(=O)[nH]2)c2oc(-c3cccnc3F)c(C)c(=O)c2c1] | 509 [M + H]⁺ |
| 5A | 3-[6-Chloro-3-[(1R)-1-[2-(5- fluoro-3-pyridyl)-3,6- dimethyl-4-oxo-chromen-8- yl]ethoxy]-2-pyridyl]-4H- 1,2,4-oxadiazol-5-one | 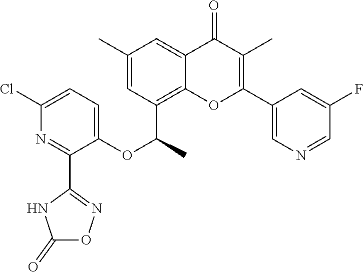 [SMILES: Cc1cc([C@@H](C)Oc2ccc(Cl)nc2-c2noc(=O)[nH]2)c2oc(-c3cncc(F)c3)c(C)c(=O)c2c1] | 509 [M + H]⁺ |
| 6A | 3-[6-Chloro-3-[(1R)-1-[2-(2,5- difluoro-3-pyridyl)-3,6- dimethyl-4-oxo-chromen-8- yl]ethoxy]-2-pyridyl]-4H- 1,2,4-oxadiazol-5-one | 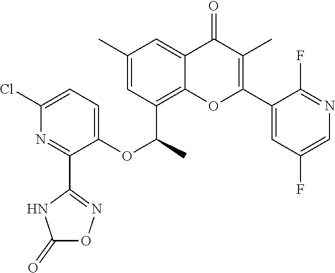 [SMILES: Cc1cc([C@@H](C)Oc2ccc(Cl)nc2-c2noc(=O)[nH]2)c2oc(-c3cc(F)cnc3F)c(C)c(=O)c2c1] | 527 [M + H]⁺ |
| 7A | 3-[6-Chloro-3-[(1R)-1-[2-(3- fluoro-2-pyridyl)-3,6- dimethyl-4-oxo-chromen-8- yl]ethoxy]-2-pyridyl]-4H- 1,2,4-oxadiazol-5-one | 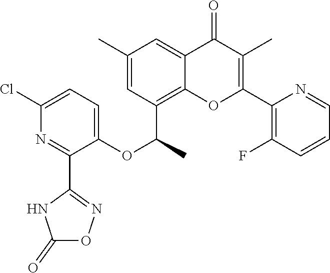 [SMILES: Cc1cc([C@@H](C)Oc2ccc(Cl)nc2-c2noc(=O)[nH]2)c2oc(-c3ncccc3F)c(C)c(=O)c2c1] | 509 [M + H]⁺ |
| 8A | 3-[6-Chloro-3-[(1R)-1-[2-(6- fluoro-2-pyridyl)-3,6- dimethyl-4-oxo-chromen-8- yl]ethoxy]-2-pyridyl]-4H- 1,2,4-oxadiazol-5-one | 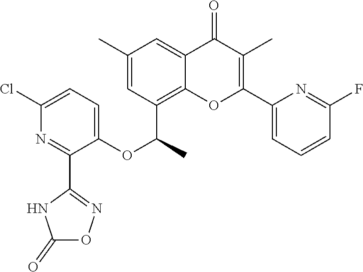 [SMILES: Cc1cc([C@@H](C)Oc2ccc(Cl)nc2-c2noc(=O)[nH]2)c2oc(-c3cccc(F)n3)c(C)c(=O)c2c1] | 509 [M + H]⁺ |
| 18A | 3-[6-Chloro-3-[(1R)-1-[3,6- dimethyl-2-(2-methylindazol- 5-yl)-4-oxo-chromen-8- yl]ethoxy]-2-pyridyl]-4H- 1,2,4-oxadiazol-5-one | 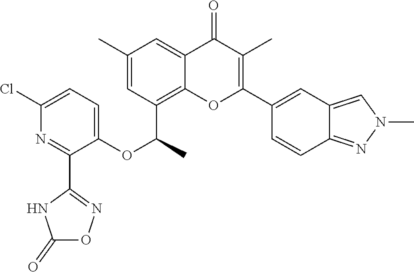 [SMILES: Cc1cc([C@@H](C)Oc2ccc(Cl)nc2-c2noc(=O)[nH]2)c2oc(-c3ccc4nn(C)cc4c3)c(C)c(=O)c2c1] | 544 [M + H]⁺ |
| 34A | 3-[6-Chloro-3-[(1R)-1-[3,6- dimethyl-2-(2-methyltriazol-4- yl)-4-oxo-chromen-8- yl]ethoxy]-2-pyridyl]-4H- 1,2,4-oxadiazol-5-one | 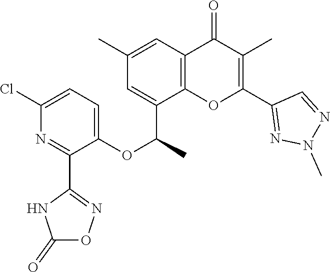 [SMILES: Cc1cc([C@@H](C)Oc2ccc(Cl)nc2-c2noc(=O)[nH]2)c2oc(-c3cnn(C)n3)c(C)c(=O)c2c1] | 495 [M + H]⁺ |
| 39A | 3-[6-Chloro-3-[(1R)-1-[2- ([1,3]dioxolo[4,5-b]pyridin-6- yl)-3,6-dimethyl-4-oxo- chromen-8-yl]ethoxy]-2- pyridyl]-4H-1,2,4-oxadiazol-5- one | 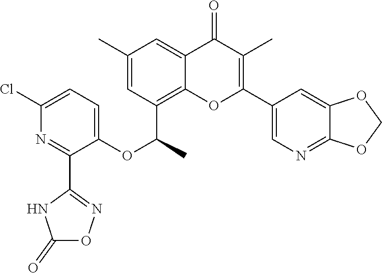 [SMILES: Cc1cc([C@@H](C)Oc2ccc(Cl)nc2-c2noc(=O)[nH]2)c2oc(-c3cnc4c(c3)OCO4)c(C)c(=O)c2c1] | 535 [M + H]⁺ |
| 41A | 3-[6-Chloro-3-[(1R)-1-[2-(6,7- dihydro-4H-pyrazolo[5,1- c][1,4]oxazin-3-yl)-3,6- dimethyl-4-oxo-chromen-8- yl]ethoxy]-2-pyridyl]-4H- 1,2,4-oxadiazol-5-one | 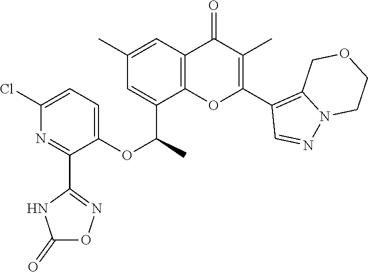 [SMILES: Cc1cc([C@@H](C)Oc2ccc(Cl)nc2-c2noc(=O)[nH]2)c2oc(-c3cnn4c3COCC4)c(C)c(=O)c2c1] | 536 [M + H]⁺ |
| 46A | 3-[6-Chloro-3-[(1R)-1-[2-(2,2- difluoro-1,3-benzodioxol-5- yl)-3,6-dimethyl-4-oxo- chromen-8-yl]ethoxy]-2- pyridyl]-4H-1,2,4-oxadiazol-5- one | 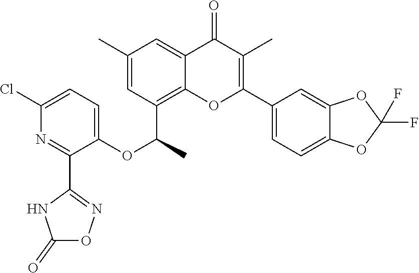 [SMILES: Cc1cc([C@@H](C)Oc2ccc(Cl)nc2-c2noc(=O)[nH]2)c2oc(-c3ccc4c(c3)OC(F)(F)O4)c(C)c(=O)c2c1] | 570 [M + H]⁺ |
| 47A | 3-[6-Chloro-3-[(1R)-1-[2-(2,3- dihydro-[1,4]dioxino[2,3- b]pyridin-7-yl)-3,6-dimethyl- 4-oxo-chromen-8-yl]ethoxy]- 2-pyridyl]-4H-1,2,4-oxadiazol- 5-one | 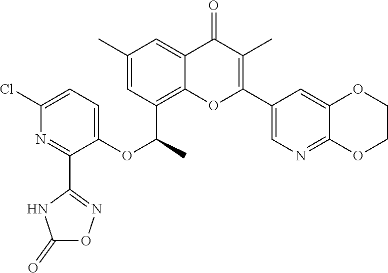 [SMILES: Cc1cc([C@@H](C)Oc2ccc(Cl)nc2-c2noc(=O)[nH]2)c2oc(-c3cnc4c(c3)OCCO4)c(C)c(=O)c2c1] | 549 [M + H]⁺ |
| 48A | 3-[6-Chloro-3-[(1R)-1-[3,6- dimethyl-2-(4-methyl-2,3- dihydro-1,4-benzoxazin-7-yl)- 4-oxo-chromen-8-yl]ethoxy]- 2-pyridyl]-4H-1,2,4-oxadiazol- 5-one | 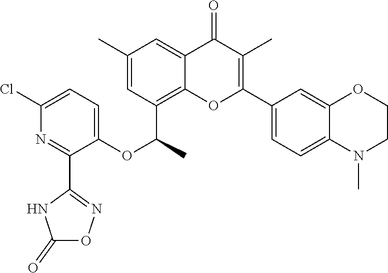 [SMILES: Cc1cc([C@@H](C)Oc2ccc(Cl)nc2-c2noc(=O)[nH]2)c2oc(-c3ccc4c(c3)OCCN4C)c(C)c(=O)c2c1] | 561 [M + H]⁺ |
| 49A | 3-[6-Chloro-3-[(1R)-1-[3,6- dimethyl-2-(4-methyl-2,3- dihydropyrido[3,2- b][1,4]oxazin-7-yl)-4-oxo- chromen-8-yl]ethoxy]-2- pyridyl]-4H-1,2,4-oxadiazol-5- lone | 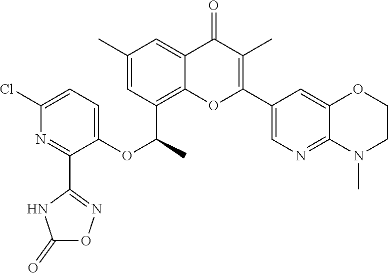 [SMILES: Cc1cc([C@@H](C)Oc2ccc(Cl)nc2-c2noc(=O)[nH]2)c2oc(-c3cnc4c(c3)OCCN4C)c(C)c(=O)c2c1] | 562 [M + H]⁺ |
| 54A | 3-[6-Chloro-3-[(1R)-1-[3,6- dimethyl-2-(1,5-naphthyridin- 3-yl)-4-oxo-chromen-8- yl]ethoxy]-2-pyridyl]-4H- 1,2,4-oxadiazol-5-one | 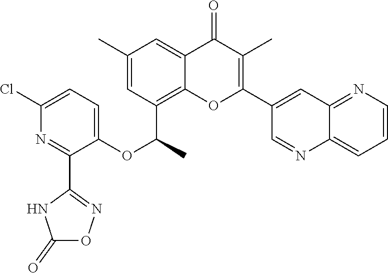 [SMILES: Cc1cc([C@@H](C)Oc2ccc(Cl)nc2-c2noc(=O)[nH]2)c2oc(-c3cnc4cccnc4c3)c(C)c(=O)c2c1] | 542 [M + H]⁺ |
| 64A | 3-[6-Chloro-3-[(1R)-1-(2- cyclopropyl-3,6-dimethyl-4- oxo-chromen-8-yl)ethoxy]-2- pyridyl]-4H-1,2,4-oxadiazol-5- lone | 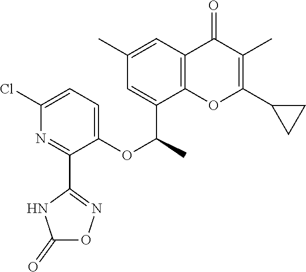 [SMILES: Cc1cc([C@@H](C)Oc2ccc(Cl)nc2-c2noc(=O)[nH]2)c2oc(C3CC3)c(C)c(=O)c2c1] | 454 [M + H]⁺ |
| 102A | 3-[6-Chloro-3-[(1R)-1-[2-(5,6- dihydro-4H-pyrrolo[2,1- e]pyrazol-3-yl)-3,6-dimethyl- 4-oxo-chromen-8-yl]ethoxy]- 2-pyridyl]-4H-1,2,4-oxadiazol- 5-one | 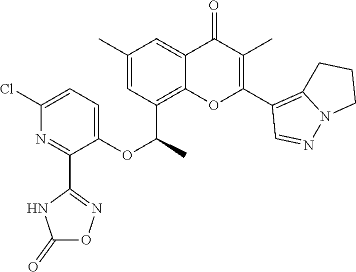 [SMILES: Cc1cc([C@@H](C)Oc2ccc(Cl)nc2-c2noc(=O)[nH]2)c2oc(-c3cnn4c3CCC4)c(C)c(=O)c2c1] | 520 [M + H]⁺ |
| 103A | 3-[3-[(1R)-1-[3,6-Dimethyl-2- (2-methylindazol-5-yl)-4-oxo- chromen-8-yl]ethoxy]-6- fluoro-2-pyridyl]-4H-1,2,4- oxadiazol-5-one | 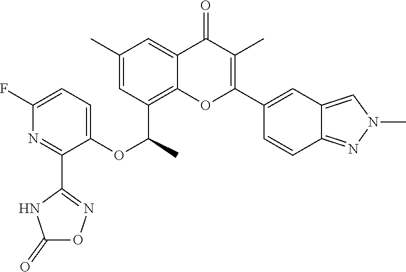 [SMILES: Cc1cc([C@@H](C)Oc2ccc(F)nc2-c2noc(=O)[nH]2)c2oc(-c3ccc4nn(C)cc4c3)c(C)c(=O)c2c1] | 528 [M + H]⁺ |
| 1B | 3-[6-Chloro-3-[(1R)-1-(3,6- dimethyl-4-oxo-2-thiazolo[5,4- b]pyridin-6-yl-chromen-8- yl)ethoxy]-2-pyridyl]-4H- 1,2,4-oxadiazol-5-one | 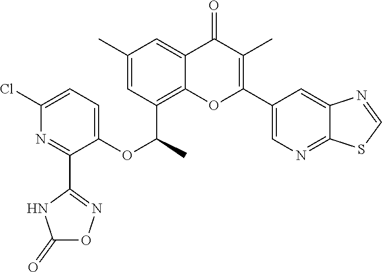 [SMILES: Cc1cc([C@@H](C)Oc2ccc(Cl)nc2-c2noc(=O)[nH]2)c2oc(-c3cnc4scnc4c3)c(C)c(=O)c2c1] | 548 [M + H]⁺ |
| 1C | 3-[6-Chloro-3-[(1R)-1-[3,6- dimethyl-2-(2- methylpyrazolo[4,3-b]pyridin- 5-yl)-4-oxo-chromen-8- yl]ethoxy]-2-pyridyl]-4H- 1,2,4-oxadiazol-5-one | 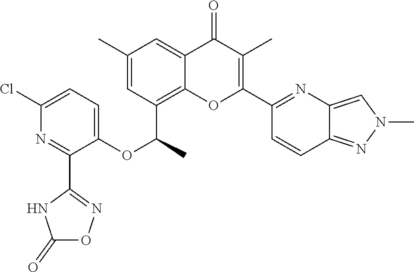 [SMILES: Cc1cc([C@@H](C)Oc2ccc(Cl)nc2-c2noc(=O)[nH]2)c2oc(-c3ccc4nn(C)cc4n3)c(C)c(=O)c2c1] | 545 [M + H]⁺ |
| 2C | 3-[6-Chloro-3-[(1R)-1-[3,6- dimethyl-2-(1- methylpyrazolo[3,4-b]pyridin- 6-yl)-4-oxo-chromen-8- yl]ethoxy]-2-pyridyl]-4H- 1,2,4-oxadiazol-5-one | 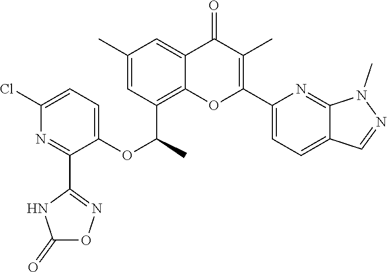 [SMILES: Cc1cc([C@@H](C)Oc2ccc(Cl)nc2-c2noc(=O)[nH]2)c2oc(-c3ccc4cnn(C)c4n3)c(C)c(=O)c2c1] | 545 [M + H]⁺ |
| 3C | 3-[6-Chloro-3-[(1R)-1-(3,6- dimethyl-4-oxo-2-pyrazin-2- yl-chromen-8-yl)ethoxy]-2- pyridyl]-4H-1,2,4-oxadiazol-5- one | 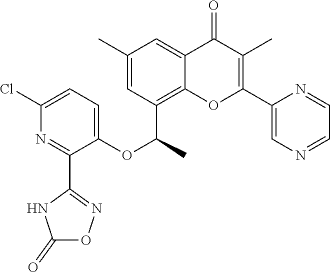 [SMILES: Cc1cc([C@@H](C)Oc2ccc(Cl)nc2-c2noc(=O)[nH]2)c2oc(-c3cnccn3)c(C)c(=O)c2c1] | 492 [M + H]⁺ |
| 4C | 3-[6-Chloro-3-[(1R)-1-[3,6- dimethyl-2-(2- methylimidazo[1,2-a]pyridin- 6-yl)-4-oxo-chromen-8- yl]ethoxy]-2-pyridyl]-4H- 1,2,4-oxadiazol-5-one | 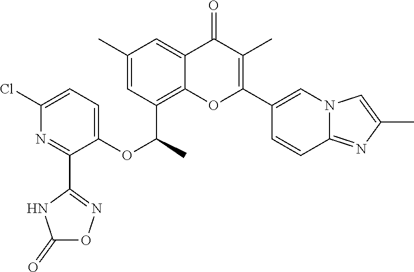 [SMILES: Cc1cc([C@@H](C)Oc2ccc(Cl)nc2-c2noc(=O)[nH]2)c2oc(-c3ccc4nc(C)cn4c3)c(C)c(=O)c2c1] | 544 [M + H]⁺ |
| 5C | 3-[6-Chloro-3-[(1R)-1-[2-(2- isopropoxypyrimidin-5-yl)- 3,6-dimethyl-4-oxo-chromen- 8-yl]ethoxy]-2-pyridyl]-4H- 1,2,4-oxadiazol-5-one | 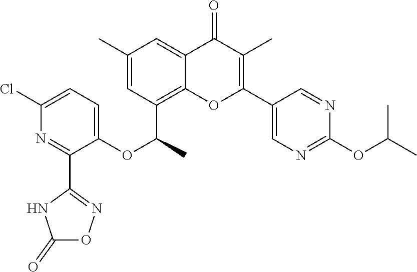 [SMILES: Cc1cc([C@@H](C)Oc2ccc(Cl)nc2-c2noc(=O)[nH]2)c2oc(-c3cnc(OC(C)C)nc3)c(C)c(=O)c2c1] | 550 [M + H]⁺ |
| 6C | 3-[6-Chloro-3-[(1R)-1-(2- furo[3,2-b]pyridin-6-yl-3,6- yl)ethoxy]-2-pyridyl]-4H- dimethyl-4-oxo-chromen-8- 1,2,4-oxadiazol-5-one | 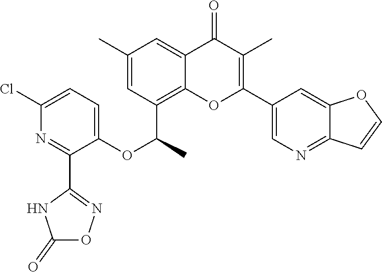 [SMILES: Cc1cc([C@@H](C)Oc2ccc(Cl)nc2-c2noc(=O)[nH]2)c2oc(-c3cnc4ccoc4c3)c(C)c(=O)c2c1] | 531 [M + H]⁺ |

### Example 9A: 2-[(1R)-1-[3,6-Dimethyl-2-(2-methylindazol-5-yl)-4-oxo-chromen-8-yl]ethoxy]benzamide

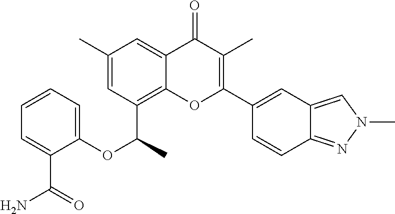 [SMILES: Cc1cc([C@@H](C)Oc2ccccc2C(N)=O)c2oc(-c3ccc4nn(C)cc4c3)c(C)c(=O)c2c1]

Dissolved 2-[(1R)-1-[3,6-dimethyl-2-(2-methylindazol-5-yl)-4-oxo-chromen-8-yl]ethoxy]benzonitrile (120 mg, 0.27 mmol) in toluene (0.7 mL) and treated with (E)-propionaldehyde oxime (58.5 mg, 0.80 mmol) and tris(triphenylphosphine)rhodium(I) chloride (Wilkinson's catalyst, 2.47 mg, 0.003 mmol). The reaction was stirred at 110° C. After cooling to rt, the reaction was concentrated under reduced pressure and the residue purified by reversed phase chromatography on C18 eluted with 10% to 45% ACN in 10 mM aqueous NH₄HCO₃ (with 5% MeOH) to give the title compound (60 mg, 46%). ES-MS m/z 466 (M−H).

The following compounds in Table 22 were made in a similar way as described for 2-[(1R)-1-[3,6-dimethyl-2-(2-methylindazol-5-yl)-4-oxo-chromen-8-yl]ethoxy]benzamide. Various methods were used to purify the compounds, which would be apparent to one skilled in the art.

TABLE 22

| | | | MS ES+ |
|---|---|---|---|
| Example # | Chemical Name | Structure | m/z |
| 10A | 3-[(1R)-1-[3,6-Dimethyl-2-(2- methylindazol-5-yl)-4-oxo- chromen-8-yl]ethoxy]pyridine- 2-carboxamide | 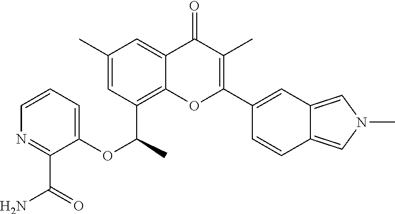 [SMILES: Cc1cc([C@@H](C)Oc2cccnc2C(N)=O)c2oc(-c3ccc4cn(C)cc4c3)c(C)c(=O)c2c1] | 467 [M − H]⁻ |
| 11A | 6-Chloro-3-[(1R)-1-[3,6- dimethyl-2-(2-methylindazol-5- yl)-4-oxo-chromen-8- yl]ethoxy]pyridine-2- carboxamide | 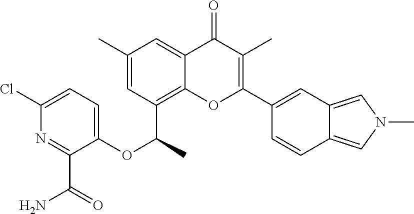 [SMILES: Cc1cc([C@@H](C)Oc2ccc(Cl)nc2C(N)=O)c2oc(-c3ccc4cn(C)cc4c3)c(C)c(=O)c2c1] | 501 [M − H]⁻ |
| 19A | 6-Chloro-3-[(1R)-1-[3,6- dimethyl-4-oxo-2-(2- pyridyl)chromen-8- yl]ethoxy]pyridine-2- carboxamide | 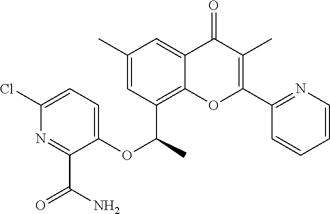 [SMILES: Cc1cc([C@@H](C)Oc2ccc(Cl)nc2C(N)=O)c2oc(-c3ccccn3)c(C)c(=O)c2c1] | 450 [M + H]⁺ |
| 20A | 6-Chloro-3-[(1R)-1-[2-(5- fluoro-3-pyridyl)-3,6-dimethyl- 4-oxo-chromen-8- yl]ethoxy]pyridine-2- carboxamide | 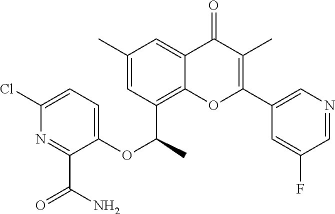 [SMILES: Cc1cc([C@@H](C)Oc2ccc(Cl)nc2C(N)=O)c2oc(-c3cncc(F)c3)c(C)c(=O)c2c1] | 468 [M + H]⁺ |
| 22A | 6-Chloro-3-[(1R)-1-[3,6- dimethyl-2-(1-methylpyrazol-4- yl)-4-oxo-chromen-8- yl]ethoxy]pyridine-2- carboxamide | 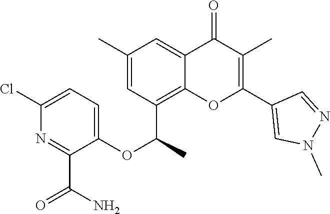 [SMILES: Cc1cc([C@@H](C)Oc2ccc(Cl)nc2C(N)=O)c2oc(-c3cnn(C)c3)c(C)c(=O)c2c1] | 453 [M + H]⁺ |
| 33A | 6-Chloro-3-[(1R)-1-[3,6- dimethyl-2-(2-methyltriazol-4- yl)-4-oxo-chromen-8- yl]ethoxy]pyridine-2- carboxamide | 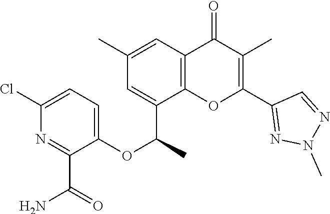 [SMILES: Cc1cc([C@@H](C)Oc2ccc(Cl)nc2C(N)=O)c2oc(-c3cnn(C)n3)c(C)c(=O)c2c1] | 454 [M + H]⁺ |
| 40A | 6-Chloro-3-[(1R)-1-[2-(6,7- dihydro-4H-pyrazolo[5,1- c][1,4]oxazin-3-yl)-3,6- dimethyl-4-oxo-chromen-8- yl]ethoxy]pyridine-2- carboxamide | 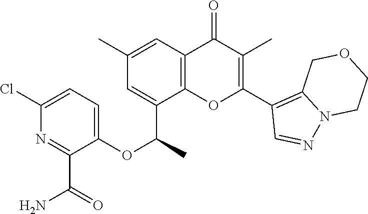 [SMILES: Cc1cc([C@@H](C)Oc2ccc(Cl)nc2C(N)=O)c2oc(-c3cnn4c3COCC4)c(C)c(=O)c2c1] | 493 [M − H]⁻ |
| 52A | 6-Chloro-3-[(1R)-1-[6-methyl- 2-(2-methylindazol-5-yl)-4- oxo-chromen-8- yl]ethoxy]pyridine-2- carboxamide | 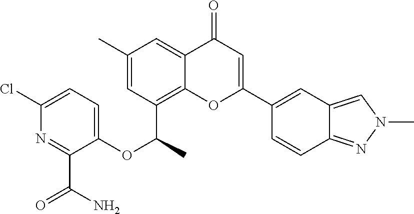 [SMILES: Cc1cc([C@@H](C)Oc2ccc(Cl)nc2C(N)=O)c2oc(-c3ccc4nn(C)cc4c3)cc(=O)c2c1] | 487 [M − H]⁻ |
| 53A | 6-Chloro-3-[(1R)-1-(6-methyl- 4-oxo-2-phenyl-chromen-8- yl)ethoxy]pyridine-2- carboxamide | 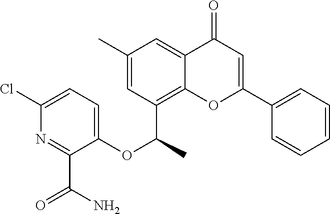 [SMILES: Cc1cc([C@@H](C)Oc2ccc(Cl)nc2C(N)=O)c2oc(-c3ccccc3)cc(=O)c2c1] | 433 [M − H]⁻ |
| 63A | 6-Chloro-3-[(1R)-1-(2- cyclopropyl-3,6-dimethyl-4- oxo-chromen-8- yl)ethoxy]pyridine-2- carboxamide | 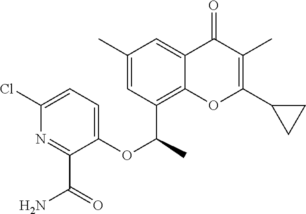 [SMILES: Cc1cc([C@@H](C)Oc2ccc(Cl)nc2C(N)=O)c2oc(C3CC3)c(C)c(=O)c2c1] | 411 [M − H]⁻ |
| 123A | 6-Chloro-3-[(1R)-1-[2-(5,6- dihydro-4H-pyrrolo[2,1- e]pyrazol-3-yl)-3,6-dimethyl-4- oxo-chromen-8- yl]ethoxy]pyridine-2- carboxamide | 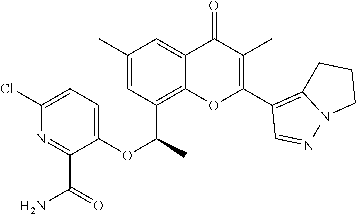 [SMILES: Cc1cc([C@@H](C)Oc2ccc(Cl)nc2C(N)=O)c2oc(-c3cnn4c3CCC4)c(C)c(=O)c2c1] | 477 [M − H]⁻ |
| 7C | 2-[(1R)-1-[3,6-Dimethyl-2-(2- methylindazol-5-yl)-4-oxo- chromen-8-yl]ethoxy]-5-fluoro- benzamide | 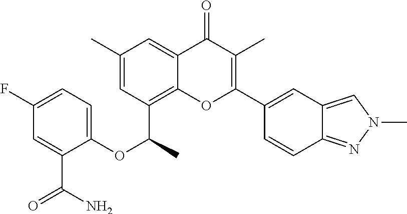 [SMILES: Cc1cc([C@@H](C)Oc2ccc(F)cc2C(N)=O)c2oc(-c3ccc4nn(C)cc4c3)c(C)c(=O)c2c1] | 484 [M − H]⁻ |

### Example 6: 6-Chloro-3-[(1R)-1-(3,6-dimethyl-4-oxo-2-phenyl-chromen-8-yl)ethoxy]-N-methylsulfonyl-pyridine-2-carboxamide

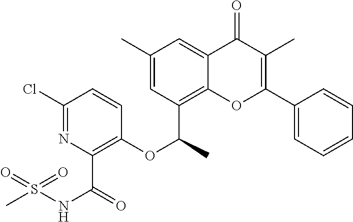 [SMILES: Cc1cc([C@@H](C)Oc2ccc(Cl)nc2C(=O)NS(C)(=O)=O)c2oc(-c3ccccc3)c(C)c(=O)c2c1]

6-Chloro-3-[(1R)-1-(3,6-dimethyl-4-oxo-2-phenyl-chromen-8-yl)ethoxy]pyridine-2-carboxylic acid (0.158 g, 0.351 mmol), 2-chloro-1-methylpyridinium iodide (0.135 g, 0.527 mmol), methanesulfonamide (0.0668 g, 0.702 mmol), and N,N-dimethylpyridin-4-amine (4.29 mg, 0.0351 mmol) were dissolved in DCM (4 mL). After 5 min, triethylamine (0.147 mL, 1.05 mmol) was added. The reaction mixture was stirred at rt overnight and concentrated under reduced pressure. The residue was purified by reversed phase chromatography eluted with a gradient of 0 to 100% ACN in H₂O+0.1% formic acid. Fractions containing the desired product were combined, washed with saturated aqueous sodium chloride, and extracted with IPA:CHCl₃ (1:3). The combined extracts were dried over MgSO₄, filtered, concentrated under reduced pressure, and the residue was recrystallized from DCM/hexanes to give the title compound (0.028 g, 15%). MS ES+ m/z 527 [M+H]⁺.

### Example 7: 8-[(1R)-1-[(6-Chloro-3-pyridyl)oxy]ethyl]-3,6-dimethyl-2-phenyl-chromen-4-one

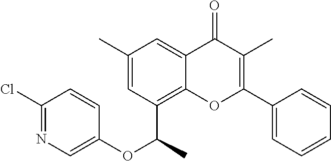 [SMILES: Cc1cc([C@@H](C)Oc2ccc(Cl)nc2)c2oc(-c3ccccc3)c(C)c(=O)c2c1]

To a solution of 8-[(1S)-1-chloroethyl]-3,6-dimethyl-2-phenyl-chromen-4-one (0.100 g, 0.320 mmol) in toluene (5 mL) was added 6-chloropyridin-3-ol (0.0828 g, 0.639 mmol) and triethylamine (0.239 mL, 1.71 mmol). The reaction mixture was stirred at 100° C. After 48 h, the reaction was concentrated under reduced pressure. The residue was purified by silica gel chromatography eluted with a gradient of 0 to 100% EtOAc in hexanes to give the title compound (0.049 g, 38%). MS ES+m/z 406 [M+H]⁺.

The following compounds in Table 23 were made in a similar way as described for 8-[(1R)-1-[(6-chloro-3-pyridyl)oxy]ethyl]-3,6-dimethyl-2-phenyl-chromen-4-one. Various methods were used to purify the compounds, which would be apparent to one skilled in the art.

TABLE 23

| | | | MS ES+ |
|---|---|---|---|
| Example # | Chemical Name | Structure | m/z |
| 8 ^{A} | 8-[1-[(6-Chloro-2- methyl-3- pyridyl)oxy]ethyl]-3,6- dimethyl-2-(3- pyridyl)chromen-4-one | 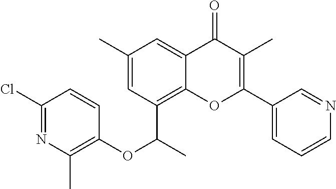 [SMILES: Cc1cc(C(C)Oc2ccc(Cl)nc2C)c2oc(-c3cccnc3)c(C)c(=O)c2c1] | 421 [M + H]⁺ |
| 9 ^{B} | 8-[1-[(6-Chloro-2- methyl-3- pyridyl)oxy]ethyl]-3,6- dimethyl-2-(3- pyridyl)chromen-4-one, Isomer 1 | 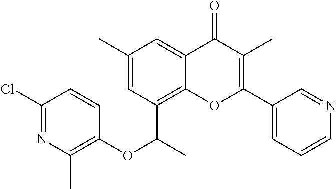 [SMILES: Cc1cc(C(C)Oc2ccc(Cl)nc2C)c2oc(-c3cccnc3)c(C)c(=O)c2c1] | 421 [M + H]⁺ |
| 10 ^{B} | 8-[1-[(6-Chloro-2- methyl-3- pyridyl)oxy]ethyl]-3,6- dimethyl-2-(3- pyridyl)chromen-4-one, Isomer 2 | 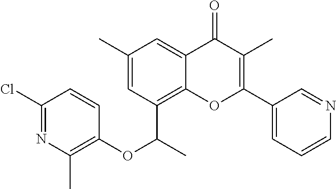 [SMILES: Cc1cc(C(C)Oc2ccc(Cl)nc2C)c2oc(-c3cccnc3)c(C)c(=O)c2c1] | 421 [M + H]⁺ |

^{A} Material was prepared from 8-[(1S)-1-chloroethyl]-3,6-dimethyl-2-(3-pyridyl)chromen-4-one; there was a loss of chiral purity during the reaction
^{B} [Chiralcel OJ-H, 21 × 150 mm; 10% MeOH in CO₂]

### Example 12A: 8-[(1R)-1-[[2-(2-Fluorophenyl)-3-pyridyl]oxy]ethyl]-3,6-dimethyl-2-phenyl-chromen-4-one

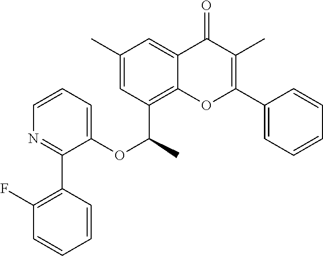 [SMILES: Cc1cc([C@@H](C)Oc2cccnc2-c2ccccc2F)c2oc(-c3ccccc3)c(C)c(=O)c2c1]

Combined 8-[(1R)-1-[(2-chloro-3-pyridyl)oxy]ethyl]-3,6-dimethyl-2-phenyl-chromen-4-one (135 mg, 0.33 mmol), (2-fluorophenyl)boronic acid (69.8 mg, 0.50 mmol), K₂CO₃ (91.9 mg, 0.67 mmol) and tetrakis(triphenylphosphine)palladium(0) (19.2 mg, 0.02 mmol) with 1,4-dioxane (2 mL) and water (0.5 mL). The reaction was sparged for 5 min with argon, and stirred at 100° C. overnight. The reaction was purified by silica gel chromatography eluted with 0% to 100% EtOAc in heptane followed by a repurification by silica gel chromatography eluted with 0% to 50% EtOAc in heptane to give the title compound (67 mg, 43%). ES/MS m/z 466.2 (M+H).

### Example 13A: 6-Chloro-3-[(1R)-1-(3,6-dimethyl-4-oxo-2-phenyl-chromen-8-yl)ethoxy]pyridine-2-sulfonamide

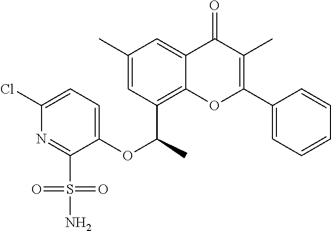 [SMILES: Cc1cc([C@@H](C)Oc2ccc(Cl)nc2S(N)(=O)=O)c2oc(-c3ccccc3)c(C)c(=O)c2c1]

A solution of (2,3,4,5,6-pentafluorophenyl) 6-chloro-3-[(1R)-1-(3,6-dimethyl-4-oxo-2-phenyl-chromen-8-yl)ethoxy]pyridine-2-sulfonate (36 mg, 0.055 mmol) in THE (1 mL) was treated with aqueous ammonia (28%, 38 μL, 0.28 mmol) and then stirred at 60° C. overnight. The reaction was concentrated under reduced pressure and purified by reversed phase chromatography on C18 eluted with 0% to 100% ACN in 10 mM aqueous NH₄HCO₃ with 5% MeOH. Fractions containing the product were pooled, washed with saturated aqueous sodium chloride, and extracted with IPA/CHCl₃ (1:3, v/v). The organics were collected, dried over MgSO₄, filtered, and concentrated under reduced pressure. The residue was crystallized from DCM/MeOH/hexane to give the title compound (22 mg, 81%) as a white solid. ES-MS m/z 485 (M+H).

The following compounds in Table 24 were made in a similar way as described for 6-chloro-3-[(1R)-1-(3,6-dimethyl-4-oxo-2-phenyl-chromen-8-yl)ethoxy]pyridine-2-sulfonamide. A base reagent such as diisopropylethylamine was sometimes used. Various methods were used to purify the compounds, which would be apparent to one skilled in the art.

TABLE 24

| | | | MS ES+ |
|---|---|---|---|
| Example # | Chemical Name | Structure | m/z |
| 14A | 6-Chloro-3-[(1R)-1-[3,6- dimethyl-2-(1-methylpyrazol-4- yl)-4-oxo-chromen-8- yl]ethoxy]pyridine-2- sulfonamide | 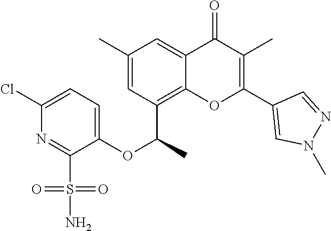 [SMILES: Cc1cc([C@@H](C)Oc2ccc(Cl)nc2S(N)(=O)=O)c2oc(-c3cnn(C)c3)c(C)c(=O)c2c1] | 489 [M + H]⁺ |
| 15A | 6-Chloro-3-[(1R)-1-[2- ([1,3]dioxolo[4,5-b]pyridin-6- yl)-3,6-dimethyl-4-oxo- chromen-8-yl]ethoxy]pyridine- 2-sulfonamide | 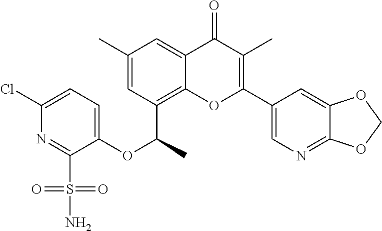 [SMILES: Cc1cc([C@@H](C)Oc2ccc(Cl)nc2S(N)(=O)=O)c2oc(-c3cnc4c(c3)OCO4)c(C)c(=O)c2c1] | 530 [M + H]⁺ |
| 16A | 6-Chloro-3-[(1R)-1-[3,6- dimethyl-2-(2-methylindazol-5- yl)-4-oxo-chromen-8- yl]ethoxy]pyridine-2- sulfonamide | 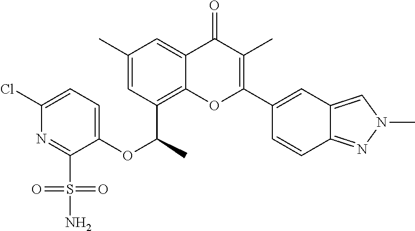 [SMILES: Cc1cc([C@@H](C)Oc2ccc(Cl)nc2S(N)(=O)=O)c2oc(-c3ccc4nn(C)cc4c3)c(C)c(=O)c2c1] | 539 [M + H]⁺ |
| 35A | 6-Chloro-3-[(1R)-1-[3,6- dimethyl-2-(2-methyltriazol-4- yl)-4-oxo-chromen-8- yl]ethoxy]pyridine-2- sulfonamide | 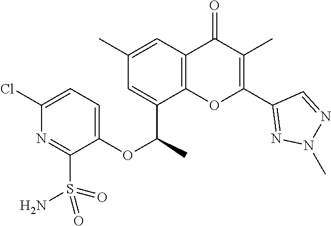 [SMILES: Cc1cc([C@@H](C)Oc2ccc(Cl)nc2S(N)(=O)=O)c2oc(-c3cnn(C)n3)c(C)c(=O)c2c1] | 490 [M + H]⁺ |
| 38A | 6-Chloro-3-[(1R)-1-(3,6- dimethyl-4-oxo-2-pyrimidin-5- yl-chromen-8- yl)ethoxy]pyridine-2- sulfonamide | 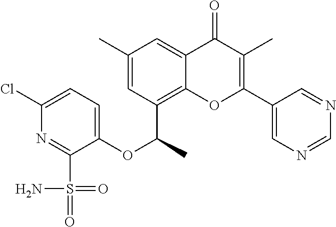 [SMILES: Cc1cc([C@@H](C)Oc2ccc(Cl)nc2S(N)(=O)=O)c2oc(-c3cncnc3)c(C)c(=O)c2c1] | 487 [M + H]⁺ |
| 42A | 6-Chloro-3-[(1R)-1-[3,6- dimethyl-4-oxo-2-(3- pyridyl)chromen-8- yl]ethoxy]pyridine-2- sulfonamide | 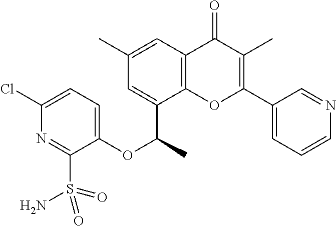 [SMILES: Cc1cc([C@@H](C)Oc2ccc(Cl)nc2S(N)(=O)=O)c2oc(-c3cccnc3)c(C)c(=O)c2c1] | 486 [M + H]⁺ |
| 43A | 6-Chloro-N-cyclopropyl-3- [(1R)-1-(3,6-dimethyl-4-oxo-2- phenyl-chromen-8- yl)ethoxy]pyridine-2- sulfonamide | 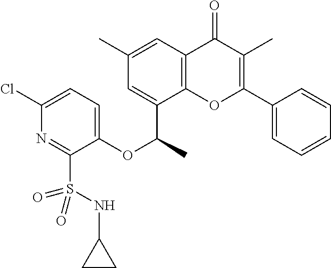 [SMILES: Cc1cc([C@@H](C)Oc2ccc(Cl)nc2S(=O)(=O)NC2CC2)c2oc(-c3ccccc3)c(C)c(=O)c2c1] | 525 [M + H]⁺ |
| 44A | 6-Chloro-3-[(1R)-1-(3,6- dimethyl-4-oxo-2-phenyl- chromen-8-yl)ethoxy]-N- (oxetan-3-yl)pyridine-2- sulfonamide | 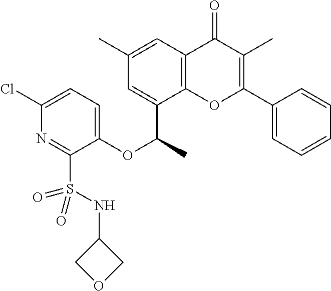 [SMILES: Cc1cc([C@@H](C)Oc2ccc(Cl)nc2S(=O)(=O)NC2COC2)c2oc(-c3ccccc3)c(C)c(=O)c2c1] | 541 [M + H]⁺ |
| 45A | 6-Chloro-3-[(1R)-1-(3,6- dimethyl-4-oxo-2-phenyl- chromen-8-yl)ethoxy]-N- (oxetan-3-ylmethyl)pyridine-2- sulfonamide | 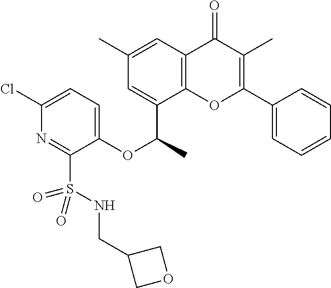 [SMILES: Cc1cc([C@@H](C)Oc2ccc(Cl)nc2S(=O)(=O)NCC2COC2)c2oc(-c3ccccc3)c(C)c(=O)c2c1] | 555 [M + H]⁺ |
| 61A | 6-Chloro-3-[(1R)-1-[2-[1-(2- hydroxyethyl)pyrazol-4-yl]-3,6- dimethyl-4-oxo-chromen-8- yl]ethoxy]pyridine-2- sulfonamide | 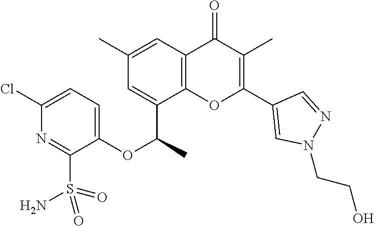 [SMILES: Cc1cc([C@@H](C)Oc2ccc(Cl)nc2S(N)(=O)=O)c2oc(-c3cnn(CCO)c3)c(C)c(=O)c2c1] | 519 [M + H]⁺ |
| 74A | 6-Chloro-3-[(1R)-1-(2- cyclopropyl-3,6-dimethyl-4- oxo-chromen-8- yl)ethoxy]pyridine-2- sulfonamide | 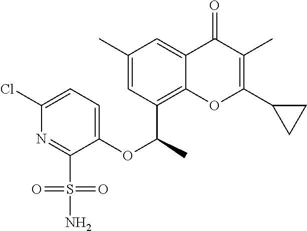 [SMILES: Cc1cc([C@@H](C)Oc2ccc(Cl)nc2S(N)(=O)=O)c2oc(C3CC3)c(C)c(=O)c2c1] | 449 [M + H]⁺ |
| 78A | 6-Chloro-3-[(1R)-1-[3,6- dimethyl-2-(2-methylpyrimidin- 5-yl)-4-oxo-chromen-8- yl]ethoxy]pyridine-2- sulfonamide | 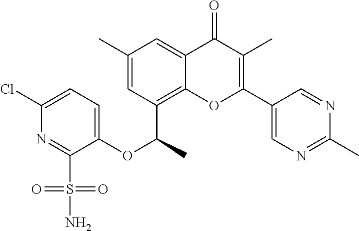 [SMILES: Cc1cc([C@@H](C)Oc2ccc(Cl)nc2S(N)(=O)=O)c2oc(-c3cnc(C)nc3)c(C)c(=O)c2c1] | 501 [M + H]⁺ |
| 83A | 6-Chloro-3-[(1R)-1-[2-(2- cyclopropylpyrimidin-5-yl)-3,6- dimethyl-4-oxo-chromen-8- yl]ethoxy]pyridine-2- sulfonamide | 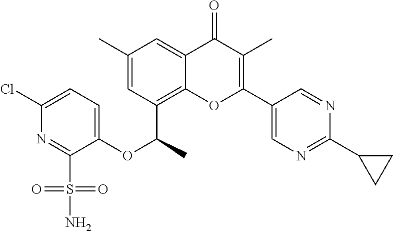 [SMILES: Cc1cc([C@@H](C)Oc2ccc(Cl)nc2S(N)(=O)=O)c2oc(-c3cnc(C4CC4)nc3)c(C)c(=O)c2c1] | 527 [M + H]⁺ |
| 2B | 6-Chloro-3-[(1R)-1-[2-[1-(2- hydroxy-2-methyl- propyl)pyrazol-4-yl]-3,6- dimethyl-4-oxo-chromen-8- yl]ethoxy]pyridine-2- sulfonamide | 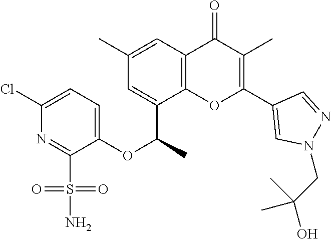 [SMILES: Cc1cc([C@@H](C)Oc2ccc(Cl)nc2S(N)(=O)=O)c2oc(-c3cnn(CC(C)(C)O)c3)c(C)c(=O)c2c1] | 547 [M + H]⁺ |
| 3B | 6-Chloro-3-[(1R)-1-[2-(6,7- dihydro-4H-pyrazolo[5,1- c][1,4]oxazin-3-yl)-3,6- dimethyl-4-oxo-chromen-8- yl]ethoxy]pyridine-2- sulfonamide | 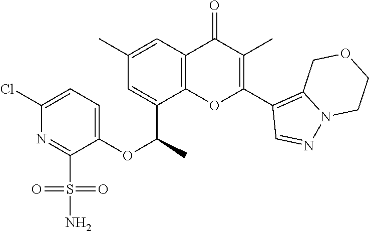 [SMILES: Cc1cc([C@@H](C)Oc2ccc(Cl)nc2S(N)(=O)=O)c2oc(-c3cnn4c3COCC4)c(C)c(=O)c2c1] | 531 [M + H]⁺ |
| 4B | 6-Chloro-3-[(1R)-1-[2-[1-[(2R)- 2-hydroxypropyl]pyrazol-4-yl]- 3,6-dimethyl-4-oxo-chromen-8- yl]ethoxy]pyridine-2- sulfonamide | 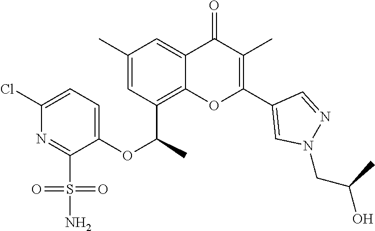 [SMILES: Cc1cc([C@@H](C)Oc2ccc(Cl)nc2S(N)(=O)=O)c2oc(-c3cnn(C[C@@H](C)O)c3)c(C)c(=O)c2c1] | 533 [M + H]⁺ |
| 5B | 6-Chloro-3-[(1R)-1-[3,6- dimethyl-2-(2- methylpyrazolo[4,3-b]pyridin- 5-yl)-4-oxo-chromen-8- yl]ethoxy]pyridine-2- sulfonamide | 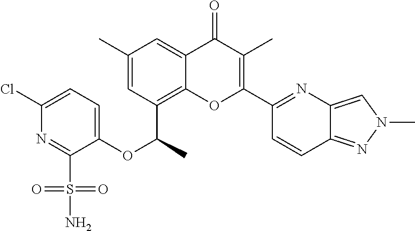 [SMILES: Cc1cc([C@@H](C)Oc2ccc(Cl)nc2S(N)(=O)=O)c2oc(-c3ccc4nn(C)cc4n3)c(C)c(=O)c2c1] | 540 [M + H]⁺ |
| 6B | 6-Chloro-3-[(1R)-1-[2-(2,3- dihydro-[1,4]dioxino[2,3- [b]pyridin-7-yl)-3,6-dimethyl-4- oxo-chromen-8- yl]ethoxy]pyridine-2- sulfonamide | 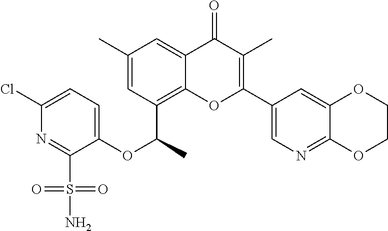 [SMILES: Cc1cc([C@@H](C)Oc2ccc(Cl)nc2S(N)(=O)=O)c2oc(-c3cnc4c(c3)OCCO4)c(C)c(=O)c2c1] | 544 [M + H]⁺ |
| 7B | 6-Chloro-3-[(1R)-1-[2-[1-[(2S)- 2-hydroxypropyl]pyrazol-4-yl]- 3,6-dimethyl-4-oxo-chromen-8- yl]ethoxy]pyridine-2- sulfonamide | 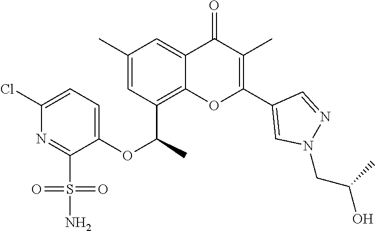 [SMILES: Cc1cc([C@@H](C)Oc2ccc(Cl)nc2S(N)(=O)=O)c2oc(-c3cnn(C[C@H](C)O)c3)c(C)c(=O)c2c1] | 533 [M + H]⁺ |

### Example 32A: 6-Chloro-3-[(1R)-1-(3,6-dimethyl-4-oxo-2-phenyl-chromen-8-yl)ethoxy]-N-methoxy-pyridine-2-sulfonamide

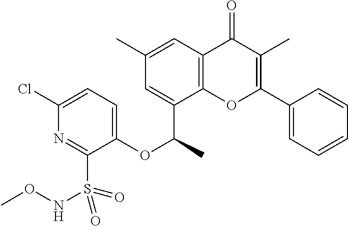 [SMILES: CONS(=O)(=O)c1nc(Cl)ccc1O[C@H](C)c1cc(C)cc2c(=O)c(C)c(-c3ccccc3)oc12]

A solution of 8-[(1R)-1-[(2-benzylsulfanyl-6-chloro-3-pyridyl)oxy]ethyl]-3,6-dimethyl-2-phenyl-chromen-4-one (0.100 g, 0.189 mmol) in AcOH/water (3:1; 2 mL) was treated with N-chlorosuccinimide (0.101 g, 0.757 mmol) at 0° C. The cooling bath was removed and the reaction stirred at rt for 2 h. The reaction was re-cooled to 0° C. and treated with O-methylhydroxylaminehydrochloride (0.158 g, 1.89 mmol) and DIPEA (0.33 mL, 1.89 mmol). The cooling bath was removed and the reaction stirred at rt for 30 min. The reaction was diluted with EtOAc and washed with saturated aqueous NaHCO₃. The aqueous layer was re-extracted with EtOAc (2×). The organics were combined, dried over Na₂SO₄, filtered, and concentrated under reduced pressure. The residue was purified by silica gel chromatography eluted with 0% to 100% (3:1 EtOAc:EtOH) in heptane and further purified twice by prep-TLC (25% EtOAc in hexanes, then 50% EtOAc in hexanes). Desired fractions were combined, concentrated, diluted with DCM/hexanes, and filtered to give the title compound (0.052 g, 54%) as a white solid. MS ES+ m z 515 [M+H]⁺.

### Example 8C: 6-Chloro-3-[(1R)-1-[3,6-dimethyl-4-oxo-2-(1H-pyrazol-5-yl)chromen-8-yl]ethoxy]pyridine-2-sulfonamide

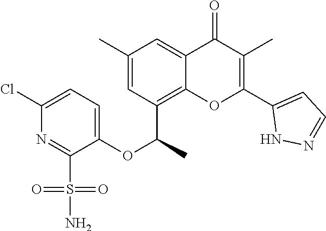 [SMILES: Cc1cc([C@@H](C)Oc2ccc(Cl)nc2S(N)(=O)=O)c2oc(-c3ccn[nH]3)c(C)c(=O)c2c1]

8-[(1R)-1-[(2-Benzylsulfanyl-6-chloro-3-pyridyl)oxy]ethyl]-3,6-dimethyl-2-(1H-pyrazol-5-yl)chromen-4-one and ammonium hydroxide were used in a manner analogous to the method of preparation of Example 32A to afford the title compound (0.002 g, 4%). MS ES+ m/z 475 [M+H]⁺.

### Example 17A: 6-[(1R)-1-[3,6-Dimethyl-2-(2-methylindazol-5-yl)-4-oxo-chromen-8-yl]ethoxy]-2,3-difluoro-benzamide

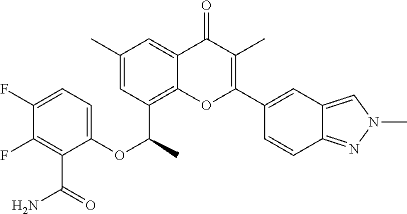 [SMILES: Cc1cc([C@@H](C)Oc2ccc(F)c(F)c2C(N)=O)c2oc(-c3ccc4nn(C)cc4c3)c(C)c(=O)c2c1]

Dissolved 6-[(1R)-1-[3,6-dimethyl-2-(2-methylindazol-5-yl)-4-oxo-chromen-8-yl]ethoxy]-2,3-difluoro-benzoic acid (50 mg, 0.1 mmol) in DMF (3 mL) and treated with HATU (57 mg, 0.15 mmol) and DIPEA (64 mg, 0.50 mmol). The reaction was stirred at rt for 10 min and then added ammonia (0.4 M in 1,4-dioxane, 1.2 mL, 0.50 mmol). The reaction was stirred at rt for 4 h. The reaction was concentrated under reduced pressure, the residue dissolved in 2 mL of DMF, and purified by reversed phase chromatography on CSH (30×150 mm, 5 μm) eluted with 30% to 60% ACN in 10 mM aqueous NH₄HCO₃ with 5% MeOH to give the title compound (6.5 mg, 13%). ES-MS m/z 502 (M−H).

The following compounds in Table 25 were made in a similar way as described for 6-[(1R)-1-[3,6-Dimethyl-2-(2-methylindazol-5-yl)-4-oxo-chromen-8-yl]ethoxy]-2,3-difluoro-benzamide. Various methods were used to purify the compounds, which would be apparent to one skilled in the art.

TABLE 25

| | | | MS ES+ |
|---|---|---|---|
| Example # | Chemical Name | Structure | m/z |
| 31A | 6-[(1R)-1-[3,6-Dimethyl-2-(1- methylpyrazol-4-yl)-4-oxo- chromen-8-yl]ethoxy]-2,3- difluoro-benzamide | 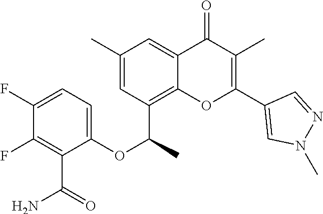 [SMILES: Cc1cc([C@@H](C)Oc2ccc(F)c(F)c2C(N)=O)c2oc(-c3cnn(C)c3)c(C)c(=O)c2c1] | 452 [M − H]⁻ |
| 36A | 6-Chloro-3-[(1R)-1-[3,6- dimethyl-2-(1-methylpyrazol-4- yl)-4-oxo-chromen-8- yl]ethoxy]-N-methoxy- pyridine-2-carboxamide | 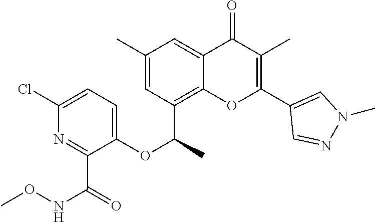 | 481 [M − H]⁻ |
| 37A | 6-[(1R)-1-[3,6-Dimethyl-2-(2- methylindazol-5-yl)-4-oxo- chromen-8-yl]ethoxy]-2,3- difluoro-N-methoxy- benzamide | 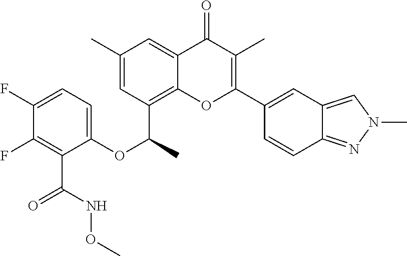 [SMILES: CONC(=O)c1c(O[C@H](C)c2cc(C)cc3c(=O)c(C)c(-c4ccc5nn(C)cc5c4)oc23)ccc(F)c1F] | 532 [M − H]⁻ |
| 55A | 6-Chloro-3-[(1R)-1-[3,6- dimethyl-4-oxo-2-(2- pyridyl)chromen-8-yl]ethoxy]- N-methoxy-pyridine-2- carboxamide | 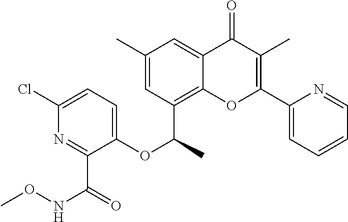 [SMILES: CONC(=O)c1nc(Cl)ccc1O[C@H](C)c1cc(C)cc2c(=O)c(C)c(-c3ccccn3)oc12] | 480 [M + H]⁺ |
| 56A | 6-Chloro-3-[(1R)-1-[2-(5- fluoro-3-pyridyl)-3,6-dimethyl- 4-oxo-chromen-8-yl]ethoxy]-N- methoxy-pyridine-2- carboxamide | 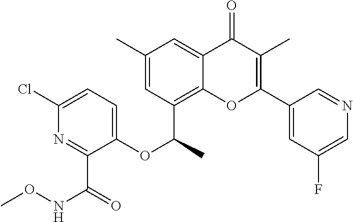 [SMILES: CONC(=O)c1nc(Cl)ccc1O[C@H](C)c1cc(C)cc2c(=O)c(C)c(-c3cncc(F)c3)oc12] | 498 [M + H]⁺ |

### Example 23A: 3-[(1R)-1-[3,6-Dimethyl-2-(2-methylindazol-5-yl)-4-oxo-chromen-8-yl]ethoxy]-N-methoxy-pyridine-2-carboxamide

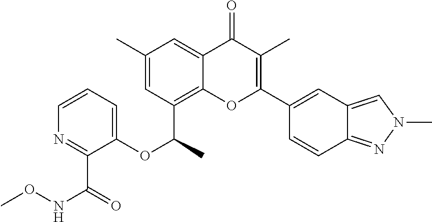

Combined 3-[(1R)-1-[3,6-dimethyl-2-(2-methylindazol-5-yl)-4-oxo-chromen-8-yl]ethoxy]pyridine-2-carboxylic acid (0.040 g, 0.085 mmol), diisopropylethylamine (0.074 mL, 0.43 mmol), O-methylhydroxylammonium chloride (0.018 g, 0.21 mmol), 4-dimethylaminopyridine (0.52 mg, 0.0043 mmol) and propylphosphonic anhydride (50 wt %, 0.14 g, 0.21 mmol) in DMF (1 mL, degassed with argon). The reaction mixture was stirred at 25° C. for 30 min, then filtered. The filtrate was concentrated under reduced pressure. The residue was purified by reversed phase chromatography eluted with 10% to 80% ACN in 0.1% aqueous formic acid to give the title compound as a white solid (0.025 mg, 55%). MS ES+ m/z 499 [M+H]⁺.

The following compounds in Table 26 were made in a similar way as described for 3-[(1R)-1-[3,6-Dimethyl-2-(2-methylindazol-5-yl)-4-oxo-chromen-8-yl]ethoxy]-N-methoxy-pyridine-2-carboxamide. Various methods were used to purify the compounds, which would be apparent to one skilled in the art.

TABLE 26

| | | | MS ES+ |
|---|---|---|---|
| Example # | Chemical Name | Structure | m/z |
| 24A | N-tert-Butoxy-3-[(1R)-1-[3,6- dimethyl-2-(2-methylindazol-5- yl)-4-oxo-chromen-8- yl]ethoxy]pyridine-2- carboxamide | 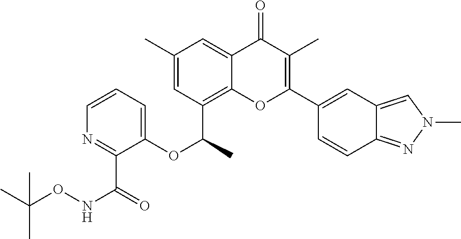 [SMILES: Cc1cc([C@@H](C)Oc2cccnc2C(=O)NOC(C)(C)C)c2oc(-c3ccc4nn(C)cc4c3)c(C)c(=O)c2c1] | 541 [M + H]⁺ |
| 25A | N-(Cyclopropylmethoxy)-3- [(1R)-1-[3,6-dimethyl-2-(2- methylindazol-5-yl)-4-oxo- chromen-8-yl]ethoxy]pyridine- 2-carboxamide | 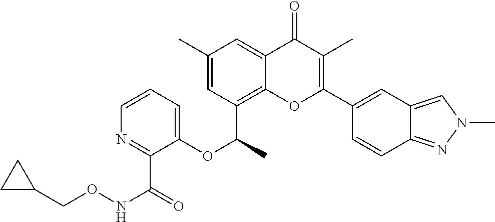 [SMILES: Cc1cc([C@@H](C)Oc2cccnc2C(=O)NOCC2CC2)c2oc(-c3ccc4nn(C)cc4c3)c(C)c(=O)c2c1] | 539 [M + H]⁺ |
| 26A | 6-Chloro-3-[(1R)-1-[3,6- dimethyl-2-(2-methylindazol-5- yl)-4-oxo-chromen-8- yl]ethoxy]-N-methoxy- pyridine-2-carboxamide | 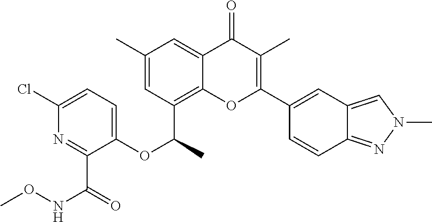 | 533 [M + H]⁺ |
| 27A | N-tert-Butoxy-6-chloro-3- [(1R)-1-[3,6-dimethyl-2-(2- methylindazol-5-yl)-4-oxo- chromen-8-yl]ethoxy]pyridine- 2-carboxamide | 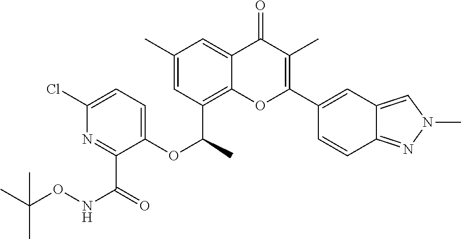 [SMILES: Cc1cc([C@@H](C)Oc2ccc(Cl)nc2C(=O)NOC(C)(C)C)c2oc(-c3ccc4nn(C)cc4c3)c(C)c(=O)c2c1] | 575 [M + H]⁺ |
| 28A | 6-Chloro-N- (cyclopropylmethoxy)-3-[(1R)- 1-[3,6-dimethyl-2-(2- methylindazol-5-yl)-4-oxo- chromen-8-yl]ethoxy]pyridine- 2-carboxamide | 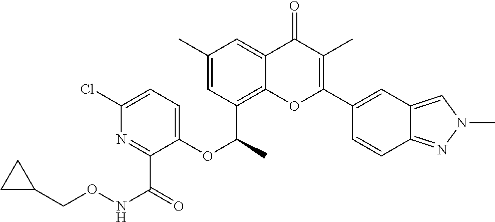 [SMILES: Cc1cc([C@@H](C)Oc2ccc(Cl)nc2C(=O)NOCC2CC2)c2oc(-c3ccc4nn(C)cc4c3)c(C)c(=O)c2c1] | 573 [M + H]⁺ |

### Example 29A: 4-[8-[(1R)-1-[[2-(2-Fluorophenyl)-3-pyridyl]oxy]ethyl]-3,6-dimethyl-4-oxo-chromen-2-yl]-1H-pyridin-2-one

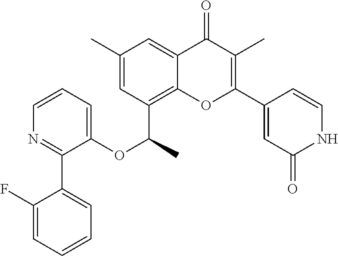 [SMILES: Cc1cc([C@@H](C)Oc2cccnc2-c2ccccc2F)c2oc(-c3cc[nH]c(=O)c3)c(C)c(=O)c2c1]

Combined 2-ethylsulfanyl-8-[(1R)-1-[[2-(2-fluorophenyl)-3-pyridyl]oxy]ethyl]-3,6-dimethyl-chromen-4-one (0.210 g, 0.467 mmol), 4-(4,4,5,5-tetramethyl-1,3,2-dioxaborolan-2-yl)-1H-pyridin-2-one (0.207 g, 0.934 mmol), copper(I) thiophene-2-carboxylate (0.178 g, 0.934 mmol) and tetrakis(triphenylphosphine)palladium(0) (0.108 g, 0.093 mmol) in EtOH (1.2 mL), degassed with argon for 5 min, and stirred at 65° C. for 16 h. The reaction mixture was filtered through diatomaceous earth and the solids washed with DCM. The combined filtrate was concentrated under reduced pressure and the residue purified by reversed phase chromatography on C18 eluted with 0% to 100% ACN in water to give the title compound (0.041 g, 17%) as a white solid. MS ES+ m/z 483 [M+H]⁺.

The following compounds in Table 27 were made in a similar way as described for 4-[8-[(1R)-1-[[2-(2-fluorophenyl)-3-pyridyl]oxy]ethyl]-3,6-dimethyl-4-oxo-chromen-2-yl]-1H-pyridin-2-one. Various methods were used to purify the compounds, which would be apparent to one skilled in the art.

TABLE 27

| | | | MS ES+ |
|---|---|---|---|
| Example # | Chemical Name | Structure | m/z |
| 21A | 8-[(1R)-1-[(6-Chloro-2- morpholino-3- pyridyl)oxy]ethyl]-3,6- dimethyl-2-(3- pyridyl)chromen-4-one | 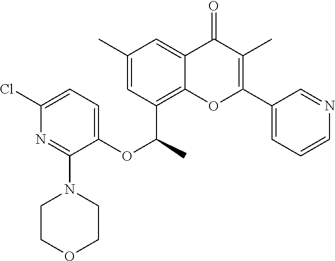 [SMILES: Cc1cc([C@@H](C)Oc2ccc(Cl)nc2N2CCOCC2)c2oc(-c3cccnc3)c(C)c(=O)c2c1] | 492 [M + H]⁺ |
| 30A | 5-[8-[(1R)-1-[2-(2- Fluorophenyl)-3- pyridyl]oxy]ethyl]-3,6- dimethyl-4-oxo-chromen-2- yl]-1H-pyridin-2-one | 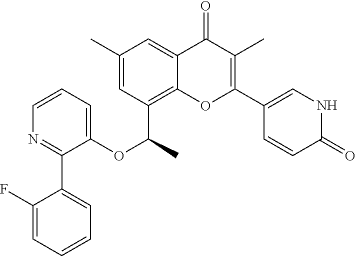 [SMILES: Cc1cc([C@@H](C)Oc2cccnc2-c2ccccc2F)c2oc(-c3ccc(=O)[nH]c3)c(C)c(=O)c2c1] | 483 [M + H]⁺ |
| 50A | 6-Chloro-3-[(1R)-1-[3,6- dimethyl-2-(2- methylbenzotriazol-5-yl)-4- oxo-chromen-8- yl]ethoxy]pyridine-2- carboxamide | 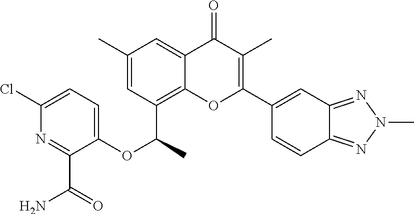 [SMILES: Cc1cc([C@@H](C)Oc2ccc(Cl)nc2C(N)=O)c2oc(-c3ccc4nn(C)nc4c3)c(C)c(=O)c2c1] | 502 [M − H]⁻ |
| 51A | 6-Chloro-3-[(1R)-1-[2-(1- cyclopropylpyrazol-4-yl)-3,6- dimethyl-4-oxo-chromen-8- yl]ethoxy]pyridine-2- carboxamide | 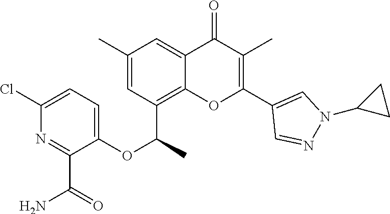 [SMILES: Cc1cc([C@@H](C)Oc2ccc(Cl)nc2C(N)=O)c2oc(-c3cnn(C4CC4)c3)c(C)c(=O)c2c1] | 477 [M − H]⁻ |
| 57A | 6-Chloro-3-[(1R)-1-[3,6- dimethyl-2-(2- methylimidazo[1,2-a]pyridin- 6-yl)-4-oxo-chromen-8- yl]ethoxy]pyridine-2- carboxamide | 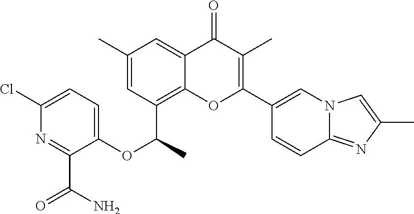 [SMILES: Cc1cc([C@@H](C)Oc2ccc(Cl)nc2C(N)=O)c2oc(-c3ccc4nc(C)cn4c3)c(C)c(=O)c2c1] | 503 [M + H]⁺ |
| 58A | 6-Chloro-3-[(1R)-1-[2-[1- (difluoromethyl)pyrazol-4- yl]-3,6-dimethyl-4-oxo- chromen-8- yl]ethoxy]pyridine-2- carboxamide | 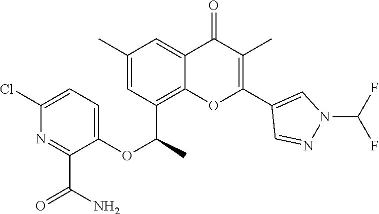 [SMILES: Cc1cc([C@@H](C)Oc2ccc(Cl)nc2C(N)=O)c2oc(-c3cnn(C(F)F)c3)c(C)c(=O)c2c1] | 489 [M + H]⁺ |
| 59A | 6-Chloro-3-[(1R)-1-[2- ([1,3]dioxolo[4,5-b]pyridin- 6-yl)-3,6-dimethyl-4-oxo- chromen-8- yl]ethoxy]pyridine-2- carboxamide | 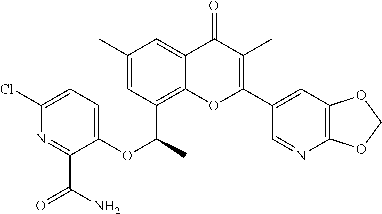 [SMILES: Cc1cc([C@@H](C)Oc2ccc(Cl)nc2C(N)=O)c2oc(-c3cnc4c(c3)OCO4)c(C)c(=O)c2c1] | 494 [M + H]⁺ |
| 60A | 6-Chloro-3-[(1R)-1-[2-(2,2- difluoro-1,3-benzodioxol-5- yl)-3,6-dimethyl-4-oxo- chromen-8- yl]ethoxy]pyridine-2- carboxamide | 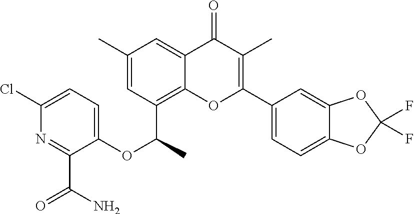 [SMILES: Cc1cc([C@@H](C)Oc2ccc(Cl)nc2C(N)=O)c2oc(-c3ccc4c(c3)OC(F)(F)O4)c(C)c(=O)c2c1] | 527 [M − H]⁻ |
| 62A | 6-Chloro-3-[(1R)-1-(3,6- dimethyl-4-oxo-2-quinoxalin- 6-yl-chromen-8- yl)ethoxy]pyridine-2- carboxamide | 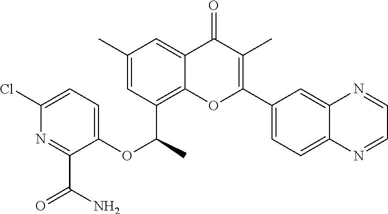 [SMILES: Cc1cc([C@@H](C)Oc2ccc(Cl)nc2C(N)=O)c2oc(-c3ccc4nccnc4c3)c(C)c(=O)c2c1] | 499 [M − H]⁻ |
| 65A | 6-Chloro-3-[(1R)-1-[2-(2,3- dihydro-1,4-benzodioxin-6- yl)-3,6-dimethyl-4-oxo- chromen-8- yl]ethoxy]pyridine-2- carboxamide | 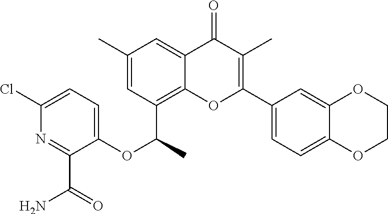 [SMILES: Cc1cc([C@@H](C)Oc2ccc(Cl)nc2C(N)=O)c2oc(-c3ccc4c(c3)OCCO4)c(C)c(=O)c2c1] | 505 [M − H]⁻ |
| 66A | 6-Chloro-3-[(1R)-1-[2-[2- (difluoromethyl)indazol-5- yl]-3,6-dimethyl-4-oxo- chromen-8- yl]ethoxy]pyridine-2- carboxamide | 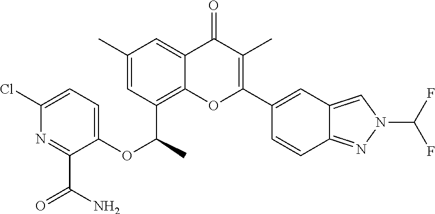 [SMILES: Cc1cc([C@@H](C)Oc2ccc(Cl)nc2C(N)=O)c2oc(-c3ccc4nn(C(F)F)cc4c3)c(C)c(=O)c2c1] | 537 [M − H]⁻ |
| 67A | 6-Chloro-3-[(1R)-1-(3,6- dimethyl-4-oxo-2-pyrimidin- 5-yl-chromen-8- yl)ethoxy]pyridine-2- carboxamide | 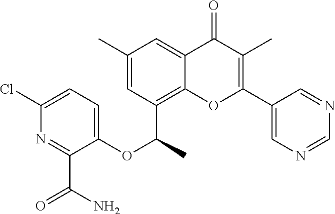 [SMILES: Cc1cc([C@@H](C)Oc2ccc(Cl)nc2C(N)=O)c2oc(-c3cncnc3)c(C)c(=O)c2c1] | 449 [M − H]⁻ |
| 68A | 6-Chloro-3-[(1R)-1-[3,6- dimethyl-2-(2-methyl-1,3- benzothiazol-6-yl)-4-oxo- chromen-8- yl]ethoxy]pyridine-2- carboxamide | 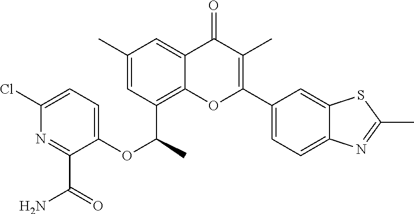 [SMILES: Cc1cc([C@@H](C)Oc2ccc(Cl)nc2C(N)=O)c2oc(-c3ccc4nc(C)sc4c3)c(C)c(=O)c2c1] | 518 [M − H]⁻ |
| 69A | 6-Chloro-3-[(1R)-1-[3,6- dimethyl-2-(1- methylbenzimidazol-5-yl)-4- oxo-chromen-8- yl]ethoxy]pyridine-2- carboxamide | 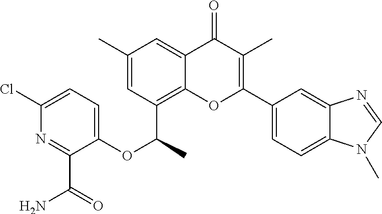 [SMILES: Cc1cc([C@@H](C)Oc2ccc(Cl)nc2C(N)=O)c2oc(-c3ccc4c(c3)ncn4C)c(C)c(=O)c2c1] | 503 [M + H]⁺ |
| 70A | 6-Chloro-3-[(1R)-1-(3,6- dimethyl-4-oxo-2-quinazolin- 7-yl-chromen-8- yl)ethoxy]pyridine-2- carboxamide | 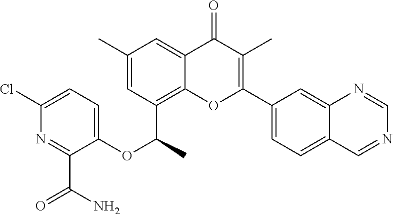 [SMILES: Cc1cc([C@@H](C)Oc2ccc(Cl)nc2C(N)=O)c2oc(-c3ccc4cncnc4c3)c(C)c(=O)c2c1] | 499 [M − H]⁻ |
| 71A | 6-Chloro-3-[(1R)-1-[3,6- dimethyl-2-(2-methyl-3,4- dihydro-1H-isoquinolin-6- yl)-4-oxo-chromen-8- yl]ethoxy]pyridine-2- carboxamide | 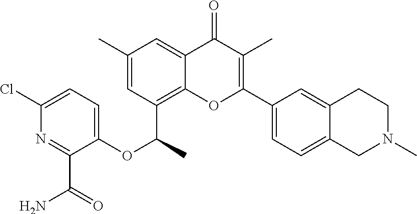 [SMILES: Cc1cc([C@@H](C)Oc2ccc(Cl)nc2C(N)=O)c2oc(-c3ccc4c(c3)CCN(C)C4)c(C)c(=O)c2c1] | 518 [M + H]⁺ |
| 104A | 6-Chloro-3-[(1R)-1-[3,6- dimethyl-2-(4-methyl-2,3- dihydro-1,4-benzoxazin-7- yl)-4-oxo-chromen-8- yl]ethoxy]pyridine-2- carboxamide | 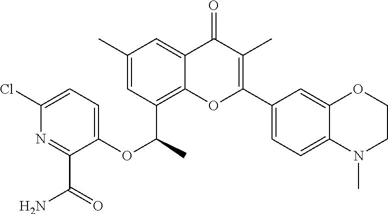 [SMILES: Cc1cc([C@@H](C)Oc2ccc(Cl)nc2C(N)=O)c2oc(-c3ccc4c(c3)OCCN4C)c(C)c(=O)c2c1] | 518 [M − H]⁻ |
| 105A | 6-Chloro-3-[(1R)-1-[3,6- dimethyl-4-oxo-2-(2-oxo-1H- quinolin-6-yl)chromen-8- yl]ethoxy]pyridine-2- carboxamide | 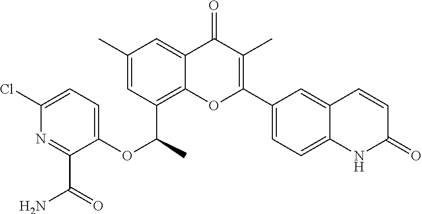 [SMILES: Cc1cc([C@@H](C)Oc2ccc(Cl)nc2C(N)=O)c2oc(-c3ccc4[nH]c(=O)ccc4c3)c(C)c(=O)c2c1] | 514 [M − H]⁻ |
| 106A | 6-Chloro-3-[(1R)-1-[3,6- dimethyl-2-(1-methyl-2-oxo- 3,4-dihydroquinolin-7-yl)-4- oxo-chromen-8- yl]ethoxy]pyridine-2- carboxamide | 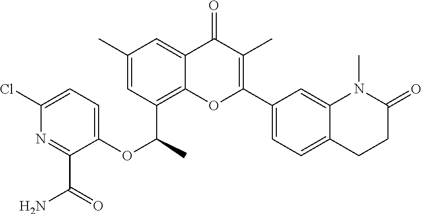 [SMILES: Cc1cc([C@@H](C)Oc2ccc(Cl)nc2C(N)=O)c2oc(-c3ccc4c(c3)N(C)C(=O)CC4)c(C)c(=O)c2c1] | 530 [M − H]⁻ |
| 107A | 6-Chloro-3-[(1R)-1-[2-[2- (dimethylamino)quinazolin- 6-yl]-3,6-dimethyl-4-oxo- chromen-8- yl]ethoxy]pyridine-2- carboxamide | 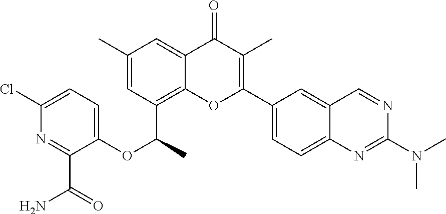 [SMILES: Cc1cc([C@@H](C)Oc2ccc(Cl)nc2C(N)=O)c2oc(-c3ccc4nc(N(C)C)ncc4c3)c(C)c(=O)c2c1] | 542 [M − H]⁻ |
| 108A | 6-Chloro-3-[(1R)-1-[2-(6,7- dihydro-5H-pyrazolo[5,1- b][1,3]oxazin-3-yl)-3,6- dimethyl-4-oxo-chromen-8- yl]ethoxy]pyridine-2- carboxamide | 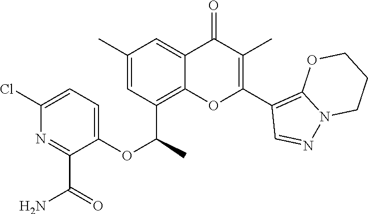 [SMILES: Cc1cc([C@@H](C)Oc2ccc(Cl)nc2C(N)=O)c2oc(-c3cnn4c3OCCC4)c(C)c(=O)c2c1] | 493 [M − H]⁻ |
| 109A | 6-Chloro-3-[(1R)-1-[3,6- dimethyl-2-(4-methyl-2,3- dihydro-1,4-benzoxazin-6- yl)-4-oxo-chromen-8- yl]ethoxy]pyridine-2- carboxamide | 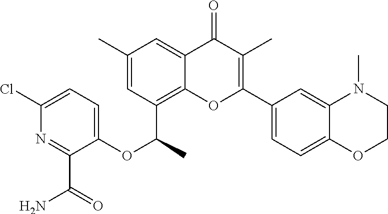 [SMILES: Cc1cc([C@@H](C)Oc2ccc(Cl)nc2C(N)=O)c2oc(-c3ccc4c(c3)N(C)CCO4)c(C)c(=O)c2c1] | 519 [M + H]⁺ |
| 110A | 6-Chloro-3-[(1R)-1-[3,6- dimethyl-2-(2-methyl-1-oxo- 3,4-dihydroisoquinolin-7-yl)- 4-oxo-chromen-8- yl]ethoxy]pyridine-2- carboxamide | 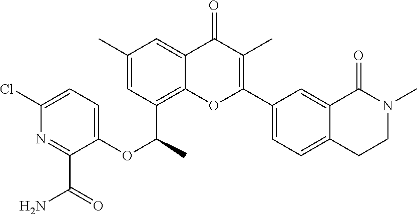 [SMILES: Cc1cc([C@@H](C)Oc2ccc(Cl)nc2C(N)=O)c2oc(-c3ccc4c(c3)C(=O)N(C)CC4)c(C)c(=O)c2c1] | 530 [M − H]⁻ |
| 111A | 6-Chloro-3-[(1R)-1-[3,6- dimethyl-2-(2-methyl-3-oxo- 1,4-dihydroisoquinolin-6-yl)- 4-oxo-chromen-8- yl]ethoxy]pyridine-2- carboxamide | 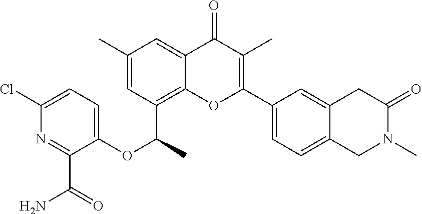 [SMILES: Cc1cc([C@@H](C)Oc2ccc(Cl)nc2C(N)=O)c2oc(-c3ccc4c(c3)CC(=O)N(C)C4)c(C)c(=O)c2c1] | 530 [M − H]⁻ |
| 112A | 6-Chloro-3-[(1R)-1-[3,6- dimethyl-2-(2-methyl-1,3- benzothiazol-5-yl)-4-oxo- chromen-8- yl]ethoxy]pyridine-2- carboxamide | 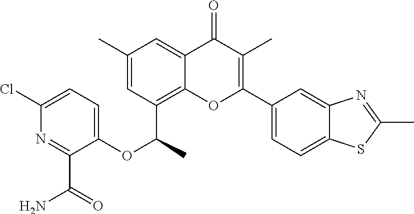 [SMILES: Cc1cc([C@@H](C)Oc2ccc(Cl)nc2C(N)=O)c2oc(-c3ccc4sc(C)nc4c3)c(C)c(=O)c2c1] | 518 [M − H]⁻ |
| 113A | 6-Chloro-3-[(1R)-1-(3,6- dimethyl-4-oxo-2- pyrazolo[1,5-a]pyridin-3-yl- chromen-8- yl)ethoxy]pyridine-2- carboxamide | 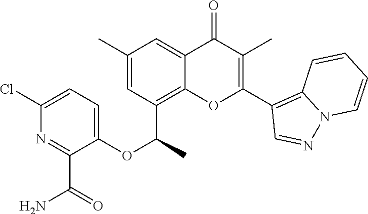 [SMILES: Cc1cc([C@@H](C)Oc2ccc(Cl)nc2C(N)=O)c2oc(-c3cnn4ccccc34)c(C)c(=O)c2c1] | 487 [M − H]⁻ |
| 114A | 6-Chloro-3-[(1R)-1-[3,6- dimethyl-2-(1-methyl-2-oxo- 3,4-dihydroquinolin-6-yl)-4- oxo-chromen-8- yl]ethoxy]pyridine-2- carboxamide | 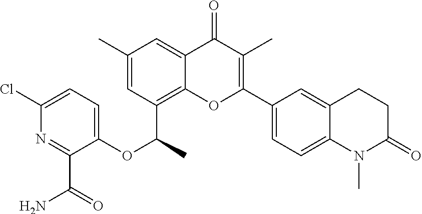 [SMILES: Cc1cc([C@@H](C)Oc2ccc(Cl)nc2C(N)=O)c2oc(-c3ccc4c(c3)CCC(=O)N4C)c(C)c(=O)c2c1] | 530 [M − H]⁻ |
| 115A | 6-Chloro-3-[(1R)-1-[3,6- dimethyl-2-(2-methyl-1-oxo- 7-isoquinolyl)-4-oxo- chromen-8- yl]ethoxy]pyridine-2- carboxamide | 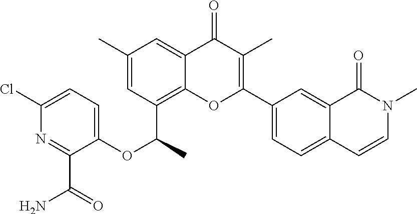 [SMILES: Cc1cc([C@@H](C)Oc2ccc(Cl)nc2C(N)=O)c2oc(-c3ccc4ccn(C)c(=O)c4c3)c(C)c(=O)c2c1] | 528 [M − H]⁻ |
| 116A | 6-Chloro-3-[(1R)-1-[3,6- dimethyl-4-oxo-2-(1-oxo-3,4- dihydro-2H-isoquinolin-6- yl)chromen-8- yl]ethoxy]pyridine-2- carboxamide | 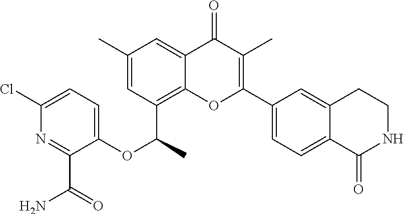 [SMILES: Cc1cc([C@@H](C)Oc2ccc(Cl)nc2C(N)=O)c2oc(-c3ccc4c(c3)CCNC4=O)c(C)c(=O)c2c1] | 516 [M − H]⁻ |
| 119A | 6-Chloro-3-[(1R)-1-[3,6- dimethyl-2-(4-methyl-3-oxo- 1,4-benzoxazin-7-yl)-4-oxo- chromen-8- yl]ethoxy]pyridine-2- carboxamide | 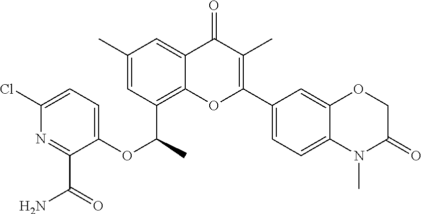 [SMILES: Cc1cc([C@@H](C)Oc2ccc(Cl)nc2C(N)=O)c2oc(-c3ccc4c(c3)OCC(=O)N4C)c(C)c(=O)c2c1] | 532 [M − H]⁻ |
| 120A | 6-Chloro-3-[(1R)-1-[3,6- dimethyl-4-oxo-2-(1-oxo-2H- isoquinolin-7-yl)chromen-8- yl]ethoxy]pyridine-2- carboxamide | 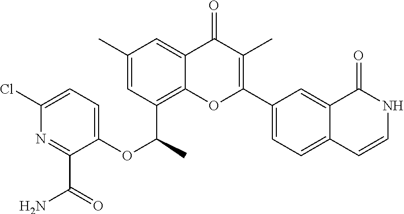 [SMILES: Cc1cc([C@@H](C)Oc2ccc(Cl)nc2C(N)=O)c2oc(-c3ccc4cc[nH]c(=O)c4c3)c(C)c(=O)c2c1] | 514 [M − H]⁻ |
| 121A | 6-Chloro-3-[(1R)-1-[3,6- dimethyl-4-oxo-2-(1-oxo-2H- isoquinolin-6-yl)chromen-8- yl]ethoxy]pyridine-2- carboxamide | 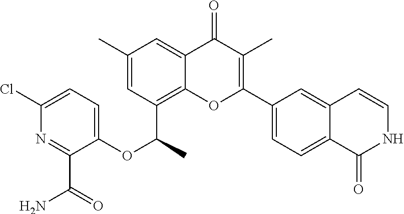 [SMILES: Cc1cc([C@@H](C)Oc2ccc(Cl)nc2C(N)=O)c2oc(-c3ccc4c(=O)[nH]ccc4c3)c(C)c(=O)c2c1] | 514 [M − H]⁻ |
| 126A | 6-Chloro-3-[(1R)-1-(3,6- dimethyl-4-oxo-2- thiazolo[5,4-b]pyridin-6-yl- chromen-8- yl)ethoxy]pyridine-2- carboxamide | 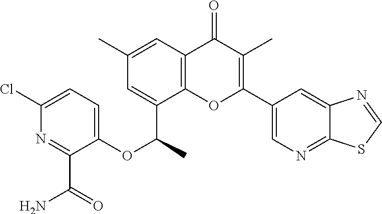 [SMILES: Cc1cc([C@@H](C)Oc2ccc(Cl)nc2C(N)=O)c2oc(-c3cnc4scnc4c3)c(C)c(=O)c2c1] | 507 [M + H]⁺ |
| 128A | 6-Chloro-3-[(1R)-1-[3,6- dimethyl-2-(2- methylpyrazolo[3,4- c]pyridin-5-yl)-4-oxo- chromen-8- yl]ethoxy]pyridine-2- carboxamide | 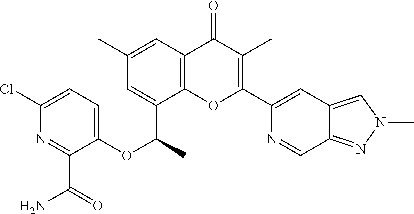 [SMILES: Cc1cc([C@@H](C)Oc2ccc(Cl)nc2C(N)=O)c2oc(-c3cc4cn(C)nc4cn3)c(C)c(=O)c2c1] | 504 [M + H]⁺ |
| 130A | 6-Chloro-3-[(1R)-1-[3,6- dimethyl-2-(2- methyloxazolo[5,4-b]pyridin- 6-yl)-4-oxo-chromen-8- yl]ethoxy]pyridine-2- carboxamide | 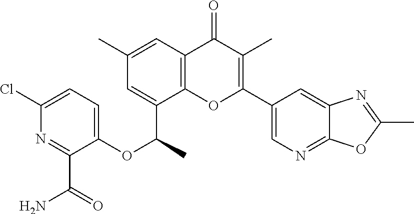 [SMILES: Cc1cc([C@@H](C)Oc2ccc(Cl)nc2C(N)=O)c2oc(-c3cnc4oc(C)nc4c3)c(C)c(=O)c2c1] | 505 [M + H]⁺ |
| 132A | 6-Chloro-3-[(1R)-1-(3,6- dimethyl-4-oxo-2- pyrazolo[1,5-a]pyrimidin-6- yl-chromen-8- yl)ethoxy]pyridine-2- carboxamide | 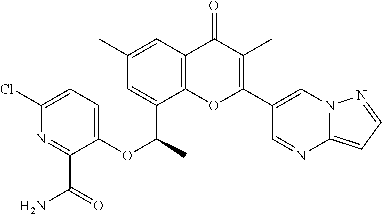 [SMILES: Cc1cc([C@@H](C)Oc2ccc(Cl)nc2C(N)=O)c2oc(-c3cnc4ccnn4c3)c(C)c(=O)c2c1] | 490 [M + H]⁺ |
| 137A | 6-Chloro-3-[(1R)-1-[3,6- dimethyl-2-(2- methylpyrazolo[3,4- b]pyridin-5-yl)-4-oxo- chromen-8- yl]ethoxy]pyridine-2- carboxamide | 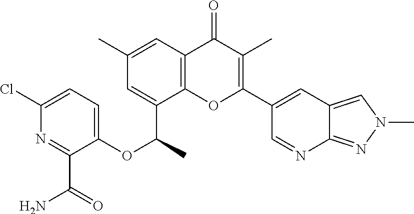 [SMILES: Cc1cc([C@@H](C)Oc2ccc(Cl)nc2C(N)=O)c2oc(-c3cnc4nn(C)cc4c3)c(C)c(=O)c2c1] | 502 [M − H]⁻ |
| 140A | 3-[(1R)-1-[2-(2,1,3- Benzothiadiazol-5-yl)-3,6- dimethyl-4-oxo-chromen-8- yl]ethoxy]-6-chloro-pyridine- 2-carboxamide | 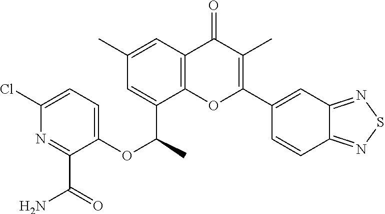 [SMILES: Cc1cc([C@@H](C)Oc2ccc(Cl)nc2C(N)=O)c2oc(-c3ccc4nsnc4c3)c(C)c(=O)c2c1] | 505 [M − H]⁻ |
| 141A | 6-Chloro-3-[(1R)-1-[3,6- dimethyl-2-(2- methylpyrazolo[4,3- b]pyridin-5-yl)-4-oxo- chromen-8- yl]ethoxy]pyridine-2- carboxamide | 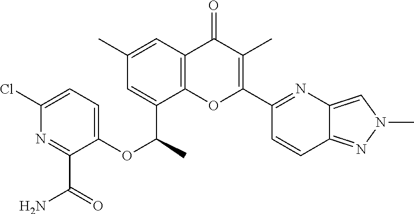 [SMILES: Cc1cc([C@@H](C)Oc2ccc(Cl)nc2C(N)=O)c2oc(-c3ccc4nn(C)cc4n3)c(C)c(=O)c2c1] | 504 [M + H]⁺ |
| 8B | 6-chloro-3-[(1R)-1-[2-(2- Methoxypyrimidin-5-yl)-3,6- dimethyl-4-oxo-chromen-8- yl]ethoxy]pyridine-2- carboxamide | 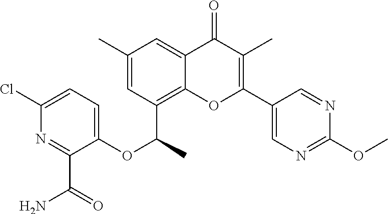 [SMILES: COc1ncc(-c2oc3c([C@@H](C)Oc4ccc(Cl)nc4C(N)=O)cc(C)cc3c(=O)c2C)cn1] | 479 [M − H]⁻ |
| 9B | 6-Chloro-3-[(1R)-1-[2-(2- cyclopropylpyrimidin-5-yl)- 3,6-dimethyl-4-oxo-chromen- 8-yl]ethoxy]pyridine-2- carboxamide | 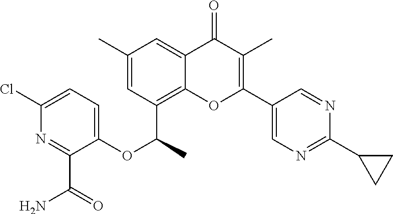 [SMILES: Cc1cc([C@@H](C)Oc2ccc(Cl)nc2C(N)=O)c2oc(-c3cnc(C4CC4)nc3)c(C)c(=O)c2c1] | 491 [M + H]⁺ |
| 10B | 6-Chloro-3-[(1R)-1-[3,6- dimethyl-4-oxo-2-(3- pyridyl)chromen-8- yl]ethoxy]pyridine-2- carboxamide | 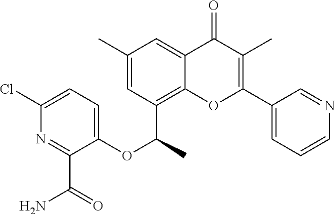 [SMILES: Cc1cc([C@@H](C)Oc2ccc(Cl)nc2C(N)=O)c2oc(-c3cccnc3)c(C)c(=O)c2c1] | 450 [M + H]⁺ |
| 11B | 3-[(1R)-1-[2-(1,2- Benzothiazol-5-yl)-3,6- dimethyl-4-oxo-chromen-8- yl]ethoxy]-6-chloro-pyridine- 2-carboxamide | 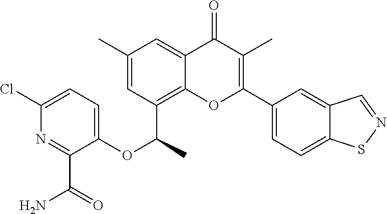 [SMILES: Cc1cc([C@@H](C)Oc2ccc(Cl)nc2C(N)=O)c2oc(-c3ccc4sncc4c3)c(C)c(=O)c2c1] | 504 [M − H]⁻ |
| 12B | 3-[(1R)-1-[2-(1,3- Benzothiazol-6-yl)-3,6- dimethyl-4-oxo-chromen-8- yl]ethoxy]-6-chloro-pyridine- 2-carboxamide | 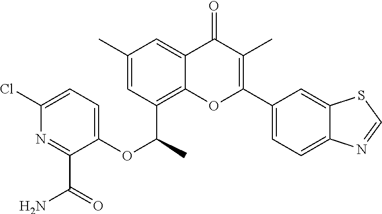 [SMILES: Cc1cc([C@@H](C)Oc2ccc(Cl)nc2C(N)=O)c2oc(-c3ccc4ncsc4c3)c(C)c(=O)c2c1] | 504 [M − H]⁻ |
| 13B | 6-Chloro-3-[(1R)-1-(3,6- dimethyl-4-oxo-2- pyrazolo[1,5-a]pyrazin-3-yl- chromen-8- yl)ethoxy]pyridine-2- carboxamide | 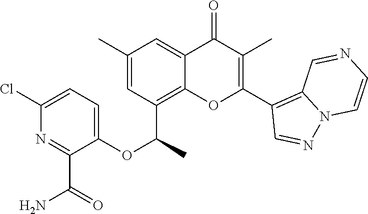 [SMILES: Cc1cc([C@@H](C)Oc2ccc(Cl)nc2C(N)=O)c2oc(-c3cnn4ccncc34)c(C)c(=O)c2c1] | 488 [M − H]⁻ |
| 14B | 6-Chloro-3-[(1R)-1-[3,6- dimethyl-2-(2-methyl-1-oxo- isoindolin-5-yl)-4-oxo- chromen-8- yl]ethoxy]pyridine-2- carboxamide | 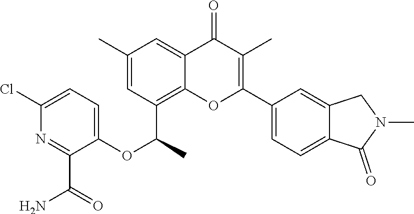 [SMILES: Cc1cc([C@@H](C)Oc2ccc(Cl)nc2C(N)=O)c2oc(-c3ccc4c(c3)CN(C)C4=O)c(C)c(=O)c2c1] | 516 [M − H]⁻ |
| 15B | 6-Chloro-3-[(1R)-1-[3,6- dimethyl-2-(2-methyl-3-oxo- isoindolin-5-yl)-4-oxo- chromen-8- yl]ethoxy]pyridine-2- carboxamide | 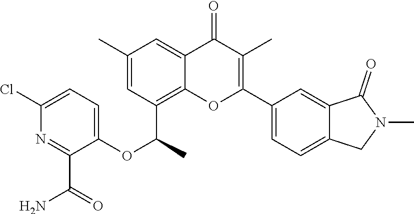 [SMILES: Cc1cc([C@@H](C)Oc2ccc(Cl)nc2C(N)=O)c2oc(-c3ccc4c(c3)C(=O)N(C)C4)c(C)c(=O)c2c1] | 516 [M − H]⁻ |
| 16B | 6-Chloro-3-[(1R)-1-(3,6- dimethyl-4-oxo-2- pyrazolo[1,5-a]pyrimidin-3- yl-chromen-8- yl)ethoxy]pyridine-2- carboxamide | 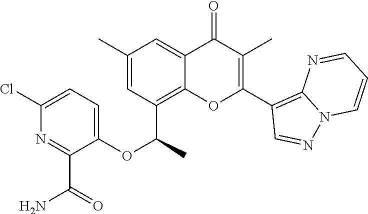 [SMILES: Cc1cc([C@@H](C)Oc2ccc(Cl)nc2C(N)=O)c2oc(-c3cnn4cccnc34)c(C)c(=O)c2c1] | 488 [M − H]⁻ |
| 17B | 6-Chloro-3-[(1R)-1-[2-(7- fluoro-2-methyl-indazol-5- yl)-3,6-dimethyl-4-oxo- chromen-8- yl]ethoxy]pyridine-2- carboxamide | 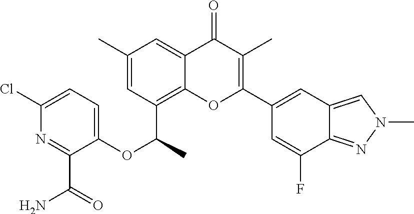 [SMILES: Cc1cc([C@@H](C)Oc2ccc(Cl)nc2C(N)=O)c2oc(-c3cc(F)c4nn(C)cc4c3)c(C)c(=O)c2c1] | 519 [M − H]⁻ |
| 18B | 6-Chloro-3-[(1R)-1-[2-(1,2- dimethylbenzimidazol-5-yl)- 3,6-dimethyl-4-oxo-chromen- 8-yl]ethoxy]pyridine-2- carboxamide | 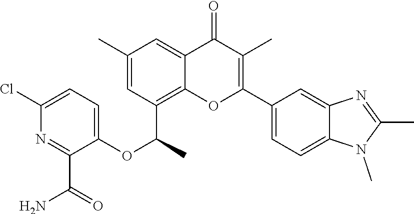 [SMILES: Cc1cc([C@@H](C)Oc2ccc(Cl)nc2C(N)=O)c2oc(-c3ccc4c(c3)nc(C)n4C)c(C)c(=O)c2c1] | 517 [M + H]⁺ |
| 19B | 6-Fluoro-3-[(1R)-1-[6- methyl-2-(2-methylindazol-5- yl)-4-oxo-chromen-8- yl]ethoxy]pyridine-2- carboxamide | 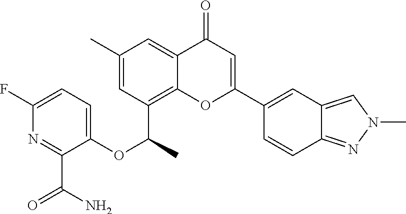 [SMILES: Cc1cc([C@@H](C)Oc2ccc(F)nc2C(N)=O)c2oc(-c3ccc4nn(C)cc4c3)cc(=O)c2c1] | 471 [M − H]⁻ |
| 9C | 6-Chloro-3-[(1R)-1-(2- isothiazol-4-yl-3,6-dimethyl- 4-oxo-chromen-8- yl)ethoxy]pyridine-2- sulfonamide | 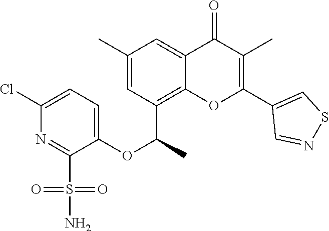 [SMILES: Cc1cc([C@@H](C)Oc2ccc(Cl)nc2S(N)(=O)=O)c2oc(-c3cnsc3)c(C)c(=O)c2c1] | 492 [M + H]⁺ |
| 10C | 6-Chloro-3-[(1R)-1-[3,6- dimethyl-2-(2-methyloxazol- 5-yl)-4-oxo-chromen-8- yl]ethoxy]pyridine-2- sulfonamide | 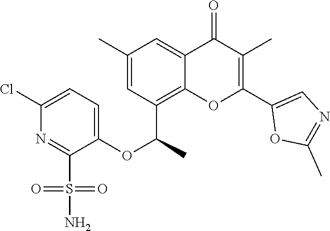 [SMILES: Cc1cc([C@@H](C)Oc2ccc(Cl)nc2S(N)(=O)=O)c2oc(-c3cnc(C)o3)c(C)c(=O)c2c1] | 490 [M + H]⁺ |
| 11C | 6-Chloro-3-[(1R)-1-[3,6- dimethyl-2-(4-methyloxazol- 5-yl)-4-oxo-chromen-8- yl]ethoxy]pyridine-2- sulfonamide | 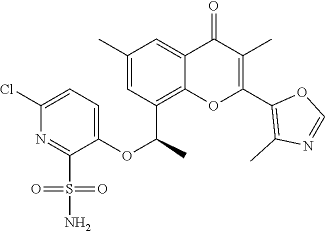 [SMILES: Cc1cc([C@@H](C)Oc2ccc(Cl)nc2S(N)(=O)=O)c2oc(-c3ocnc3C)c(C)c(=O)c2c1] | 490 [M + H]⁺ |
| 12C | 6-Chloro-3-[(1R)-1-(2- isoxazol-4-yl-3,6-dimethyl-4- oxo-chromen-8- yl)ethoxy]pyridine-2- sulfonamide | 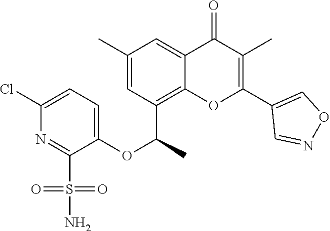 [SMILES: Cc1cc([C@@H](C)Oc2ccc(Cl)nc2S(N)(=O)=O)c2oc(-c3cnoc3)c(C)c(=O)c2c1] | 476 [M + H]⁺ |
| 13C | 6-Chloro-3-[(1R)-1-(3,6- dimethyl-4-oxo-2- thiazolo[5,4-b]pyridin-6-yl- chromen-8- yl)ethoxy]pyridine-2- sulfonamide | 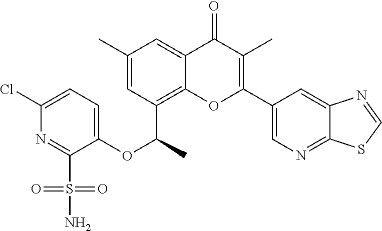 [SMILES: Cc1cc([C@@H](C)Oc2ccc(Cl)nc2S(N)(=O)=O)c2oc(-c3cnc4scnc4c3)c(C)c(=O)c2c1] | 543 [M + H]⁺ |
| 14C | 6-Chloro-3-[(1R)-1-[2-[2-(1- cyanocyclopropyl)pyrimidin- 5-yl]-3,6-dimethyl-4-oxo- chromen-8- yl]ethoxy]pyridine-2- sulfonamide | 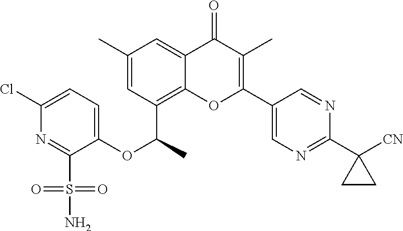 [SMILES: Cc1cc([C@@H](C)Oc2ccc(Cl)nc2S(N)(=O)=O)c2oc(-c3cnc(C4(C#N)CC4)nc3)c(C)c(=O)c2c1] | 552 [M + H]⁺ |
| 15C | 6-Chloro-3-[(1R)-1-[3,6- dimethyl-2-(2-methylpyrazol- 3-yl)-4-oxo-chromen-8- yl]ethoxy]pyridine-2- sulfonamide | 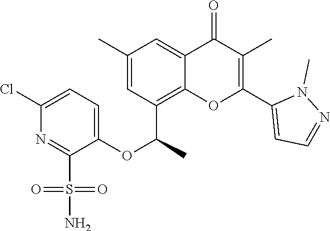 [SMILES: Cc1cc([C@@H](C)Oc2ccc(Cl)nc2S(N)(=O)=O)c2oc(-c3ccnn3C)c(C)c(=O)c2c1] | 489 [M + H]⁺ |
| 16C | 6-Chloro-3-[(1R)-1-[2-(2,5- dimethylpyrazol-3-yl)-3,6- dimethyl-4-oxo-chromen-8- yl]ethoxy]pyridine-2- sulfonamide | 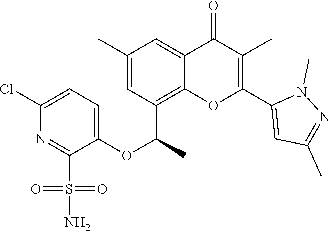 [SMILES: Cc1cc([C@@H](C)Oc2ccc(Cl)nc2S(N)(=O)=O)c2oc(-c3cc(C)nn3C)c(C)c(=O)c2c1] | 503 [M + H]⁺ |
| 17C | 6-Chloro-3-[(1R)-1-[3,6- dimethyl-2-(1-methylpyrazol- 3-yl)-4-oxo-chromen-8- yl]ethoxy]pyridine-2- sulfonamide | 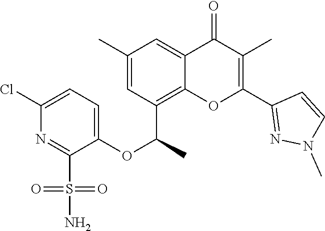 [SMILES: Cc1cc([C@@H](C)Oc2ccc(Cl)nc2S(N)(=O)=O)c2oc(-c3ccn(C)n3)c(C)c(=O)c2c1] | 489 [M + H]⁺ |
| 18C | 6-Chloro-3-[(1R)-1-[2-(1,5- dimethylpyrazol-4-yl)-3,6- dimethyl-4-oxo-chromen-8- yl]ethoxy]pyridine-2- sulfonamide | 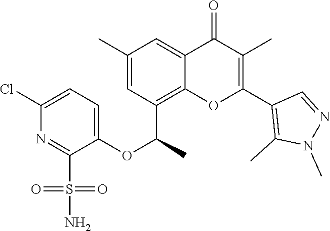 [SMILES: Cc1cc([C@@H](C)Oc2ccc(Cl)nc2S(N)(=O)=O)c2oc(-c3cnn(C)c3C)c(C)c(=O)c2c1] | 503 [M + H]⁺ |
| 19C | 6-Chloro-3-[(1R)-1-[3,6- dimethyl-2-(2- methylimidazo[1,2- b]pyridazin-6-yl)-4-oxo- chromen-8- yl]ethoxy]pyridine-2- sulfonamide | 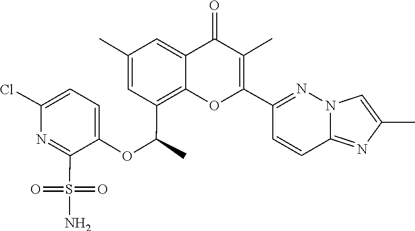 [SMILES: Cc1cc([C@@H](C)Oc2ccc(Cl)nc2S(N)(=O)=O)c2oc(-c3ccc4nc(C)cn4n3)c(C)c(=O)c2c1] | 540 [M + H]⁺ |
| 20C | 6-Chloro-3-[(1R)-1-(3,6- dimethyl-4-oxo-2- pyrazolo[1,5-a]pyridin-6-yl- chromen-8- yl)ethoxy]pyridine-2- sulfonamide | 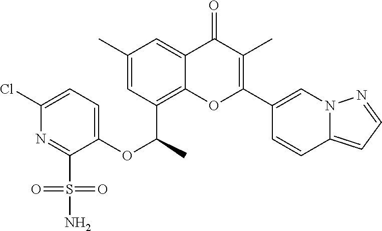 [SMILES: Cc1cc([C@@H](C)Oc2ccc(Cl)nc2S(N)(=O)=O)c2oc(-c3ccc4ccnn4c3)c(C)c(=O)c2c1] | 525 [M + H]⁺ |
| 21C | 6-Chloro-3-[(1R)-1-[2-(5- cyano-1-methyl-pyrazol-4- yl)-3,6-dimethyl-4-oxo- chromen-8- yl]ethoxy]pyridine-2- sulfonamide | 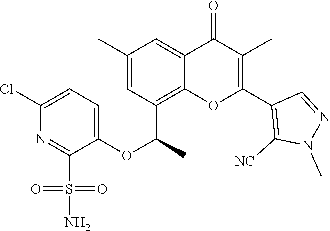 [SMILES: Cc1cc([C@@H](C)Oc2ccc(Cl)nc2S(N)(=O)=O)c2oc(-c3cnn(C)c3C#N)c(C)c(=O)c2c1] | 514 [M + H]⁺ |
| 22C | 6-Chloro-3-[(1R)-1-(2- furo[3,2-b]pyridin-6-yl-3,6- dimethyl-4-oxo-chromen-8- yl)ethoxy]pyridine-2- sulfonamide | 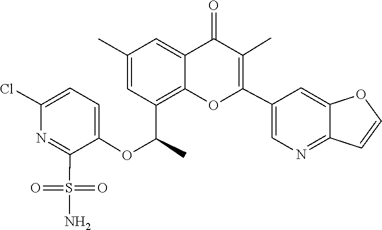 [SMILES: Cc1cc([C@@H](C)Oc2ccc(Cl)nc2S(N)(=O)=O)c2oc(-c3cnc4ccoc4c3)c(C)c(=O)c2c1] | 526 [M + H]⁺ |
| 23C | 6-Chloro-3-[(1R)-1-[2-(2- isopropoxypyrimidin-5-yl)- 3,6-dimethyl-4-oxo-chromen- 8-yl]ethoxy]pyridine-2- sulfonamide | 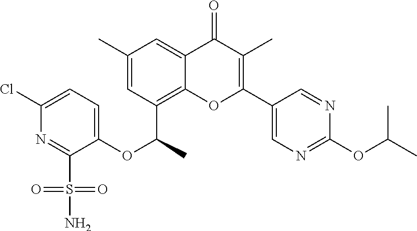 [SMILES: Cc1cc([C@@H](C)Oc2ccc(Cl)nc2S(N)(=O)=O)c2oc(-c3cnc(OC(C)C)nc3)c(C)c(=O)c2c1] | 545 [M + H]⁺ |
| 24C | 6-Chloro-3-[(1R)-1-[2-(3- cyanophenyl)-3,6-dimethyl- 4-oxo-chromen-8- yl]ethoxy]pyridine-2- sulfonamide | 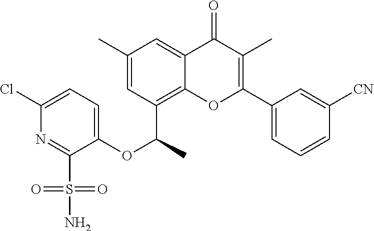 [SMILES: Cc1cc([C@@H](C)Oc2ccc(Cl)nc2S(N)(=O)=O)c2oc(-c3cccc(C#N)c3)c(C)c(=O)c2c1] | 510 [M + H]⁺ |
| 25C | 6-Chloro-3-[(1R)-1-[2-(4- cyanophenyl)-3,6-dimethyl- 4-oxo-chromen-8- yl]ethoxy]pyridine-2- sulfonamide | 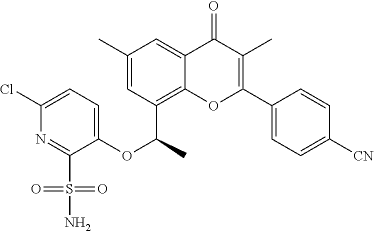 [SMILES: Cc1cc([C@@H](C)Oc2ccc(Cl)nc2S(N)(=O)=O)c2oc(-c3ccc(C#N)cc3)c(C)c(=O)c2c1] | 510 [M + H]⁺ |
| 26C | 6-Chloro-3-[(1R)-1-[3,6- dimethyl-4-oxo-2-[1-(3- pyridyl)pyrazol-4- yl]chromen-8- yl]ethoxy]pyridine-2- sulfonamide | 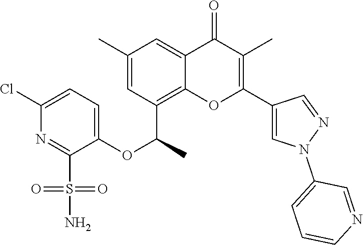 [SMILES: Cc1cc([C@@H](C)Oc2ccc(Cl)nc2S(N)(=O)=O)c2oc(-c3cnn(-c4cccnc4)c3)c(C)c(=O)c2c1] | 552 [M + H]⁺ |
| 27C | 3-[(1R)-1-[2-(2,1,3- Benzoxadiazol-5-yl)-3,6- dimethyl-4-oxo-chromen-8- yl]ethoxy]-6-chloro-pyridine- 2-sulfonamide | 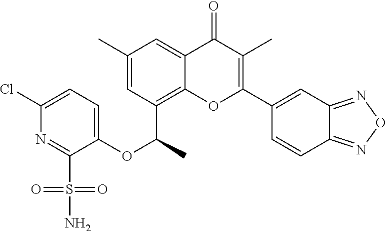 [SMILES: Cc1cc([C@@H](C)Oc2ccc(Cl)nc2S(N)(=O)=O)c2oc(-c3ccc4nonc4c3)c(C)c(=O)c2c1] | 527 [M + H]⁺ |
| 28C | 6-Chloro-3-[(1R)-1-[3,6- dimethyl-4-oxo-2-(2- pyridyl)chromen-8- yl]ethoxy]pyridine-2- sulfonamide | 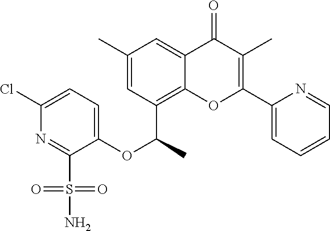 [SMILES: Cc1cc([C@@H](C)Oc2ccc(Cl)nc2S(N)(=O)=O)c2oc(-c3ccccn3)c(C)c(=O)c2c1] | 486 [M + H]⁺ |
| 29C | 6-Chloro-3-[(1R)-1-(3,6- dimethyl-4-oxo-2-pyrazin-2- yl-chromen-8- yl)ethoxy]pyridine-2- sulfonamide | 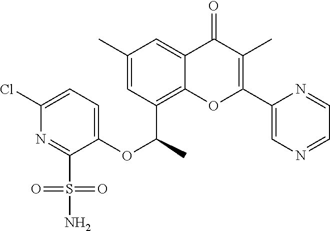 [SMILES: Cc1cc([C@@H](C)Oc2ccc(Cl)nc2S(N)(=O)=O)c2oc(-c3cnccn3)c(C)c(=O)c2c1] | 487 [M + H]⁺ |
| 30C | 6-Chloro-3-[(1R)-1-[3,6- dimethyl-2- ([1,2,5]oxadiazolo[3,4- b]pyridin-6-yl)-4-oxo- chromen-8- yl]ethoxy]pyridine-2- sulfonamide | 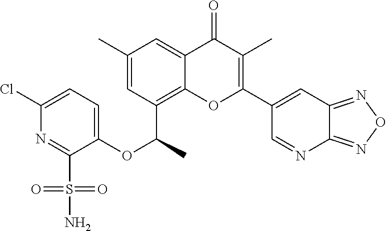 [SMILES: Cc1cc([C@@H](C)Oc2ccc(Cl)nc2S(N)(=O)=O)c2oc(-c3cnc4nonc4c3)c(C)c(=O)c2c1] | 528 [M + H]⁺ |
| 31C | 6-Chloro-3-[(1R)-1-[3,6- dimethyl-2-(4- methylsulfonylphenyl)-4- oxo-chromen-8- yl]ethoxy]pyridine-2- sulfonamide | 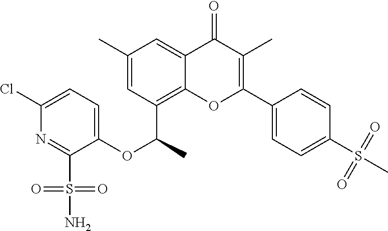 [SMILES: Cc1cc([C@@H](C)Oc2ccc(Cl)nc2S(N)(=O)=O)c2oc(-c3ccc(S(C)(=O)=O)cc3)c(C)c(=O)c2c1] | 563 [M + H]⁺ |
| 32C | 3-[(1R)-1-[2-(1,3- Benzoxazol-4-yl)-3,6- dimethyl-4-oxo-chromen-8- yl]ethoxy]-6-chloro-pyridine- 2-sulfonamide | 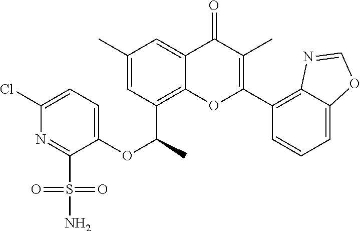 [SMILES: Cc1cc([C@@H](C)Oc2ccc(Cl)nc2S(N)(=O)=O)c2oc(-c3cccc4ocnc34)c(C)c(=O)c2c1] | 526 [M + H]⁺ |
| 33C | 6-Chloro-3-[(1R)-1-[2-(4- ethylsulfonylphenyl)-3,6- dimethyl-4-oxo-chromen-8- yl]ethoxy]pyridine-2- sulfonamide | 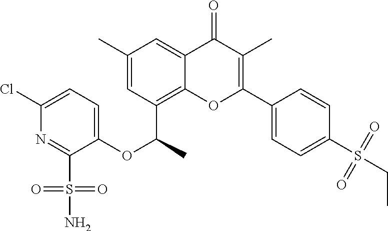 [SMILES: CCS(=O)(=O)c1ccc(-c2oc3c([C@@H](C)Oc4ccc(Cl)nc4S(N)(=O)=O)cc(C)cc3c(=O)c2C)cc1] | 577 [M + H]⁺ |
| 34C | 3-[(1R)-1-[3,6-Dimethyl-4- oxo-2-(2-pyridyl)chromen-8- yl]ethoxy]pyridine-2- sulfonamide | 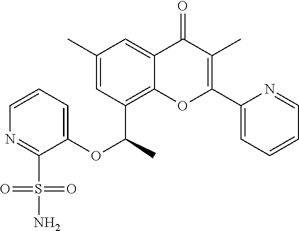 [SMILES: Cc1cc([C@@H](C)Oc2cccnc2S(N)(=O)=O)c2oc(-c3ccccn3)c(C)c(=O)c2c1] | 452 [M + H]⁺ |
| 35C | 3-[(1R)-1-(6-Methyl-4-oxo-2- phenyl-chromen-8- yl)ethoxy]pyridine-2- sulfonamide | 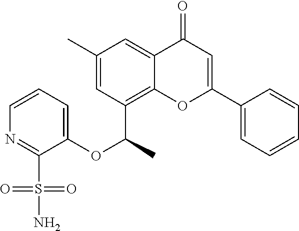 [SMILES: Cc1cc([C@@H](C)Oc2cccnc2S(N)(=O)=O)c2oc(-c3ccccc3)cc(=O)c2c1] | 437 [M + H]⁺ |
| 36C | 6-Chloro-3-[(1R)-1-[2-(2,3- dihydropyrazolo[5,1- b]oxazol-7-yl)-3,6-dimethyl- 4-oxo-chromen-8- yl]ethoxy]pyridine-2- carboxamide | 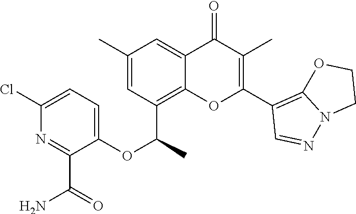 [SMILES: Cc1cc([C@@H](C)Oc2ccc(Cl)nc2C(N)=O)c2oc(-c3cnn4c3OCC4)c(C)c(=O)c2c1] | 479 [M − H]⁻ |
| 37C | 6-Chloro-3-[(1R)-1-[3,6- dimethyl-2-(2- methyloxazolo[5,4-b]pyridin- 6-yl)-4-oxo-chromen-8- yl]ethoxy]pyridine-2- carboxamide | 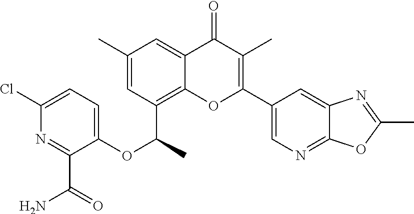 [SMILES: Cc1cc([C@@H](C)Oc2ccc(Cl)nc2C(N)=O)c2oc(-c3cnc4oc(C)nc4c3)c(C)c(=O)c2c1] | 505 [M + H]⁺ |
| 38C | 6-Chloro-3-[(1R)-1-[2-(8- fluoro-2-methyl-imidazo[1,2- alpyridin-6-yl)-3,6-dimethyl- 4-oxo-chromen-8- yl]ethoxy]pyridine-2- carboxamide | 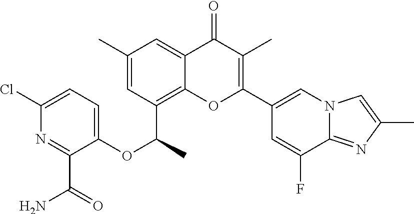 [SMILES: Cc1cc([C@@H](C)Oc2ccc(Cl)nc2C(N)=O)c2oc(-c3cc(F)c4nc(C)cn4c3)c(C)c(=O)c2c1] | 521 [M + H]⁺ |
| 39C | 6-Chloro-3-[(1R)-1-(3,6- dimethyl-4-oxo-2- pyrazolo[1,5-b]pyridazin-3- yl-chromen-8- yl)ethoxy]pyridine-2- carboxamide | 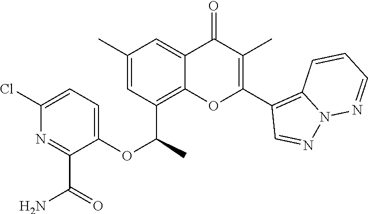 [SMILES: Cc1cc([C@@H](C)Oc2ccc(Cl)nc2C(N)=O)c2oc(-c3cnn4ncccc34)c(C)c(=O)c2c1] | 488 [M − H]⁻ |
| 40C | 6-Chloro-3-[(1R)-1-[3,6- dimethyl-2-(1-methylindazol- 5-yl)-4-oxo-chromen-8- yl]ethoxy]pyridine-2- carboxamide | 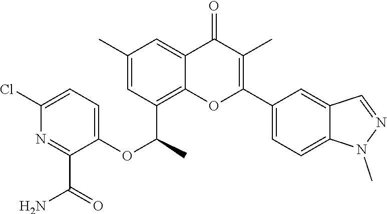 [SMILES: Cc1cc([C@@H](C)Oc2ccc(Cl)nc2C(N)=O)c2oc(-c3ccc4c(cnn4C)c3)c(C)c(=O)c2c1] | 501 [M − H]⁻ |
| 41C | 6-Chloro-3-[(1R)-1-[3,6- dimethyl-4-oxo-2-(2-oxo-1,4- dihydro-3,1-benzoxazin-6- yl)chromen-8- yl]ethoxy]pyridine-2- carboxamide | 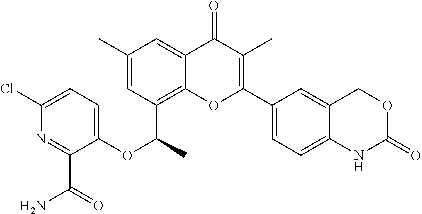 [SMILES: Cc1cc([C@@H](C)Oc2ccc(Cl)nc2C(N)=O)c2oc(-c3ccc4c(c3)COC(=O)N4)c(C)c(=O)c2c1] | 518 [M − H]⁻ |
| 42C | 6-Chloro-3-[(1R)-1-[2-(6- fluoro-2-methyl-indazol-5- yl)-3,6-dimethyl-4-oxo- chromen-8- yl]ethoxy]pyridine-2- carboxamide | 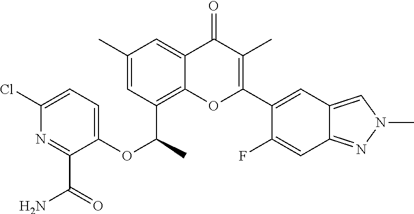 [SMILES: Cc1cc([C@@H](C)Oc2ccc(Cl)nc2C(N)=O)c2oc(-c3cc4cn(C)nc4cc3F)c(C)c(=O)c2c1] | 519 [M − H]⁻ |
| 43C | 6-Chloro-3-[(1R)-1-(2- imidazo[1,5-a]pyridin-6-yl- 3,6-dimethyl-4-oxo-chromen- 8-yl)ethoxy]pyridine-2- carboxamide | 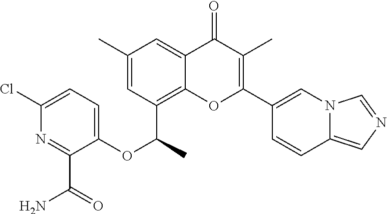 [SMILES: Cc1cc([C@@H](C)Oc2ccc(Cl)nc2C(N)=O)c2oc(-c3ccc4cncn4c3)c(C)c(=O)c2c1] | 489 [M + H]⁺ |
| 44C | 6-Chloro-3-[(1R)-1-[2-(7- methoxy-2-methyl-indazol-5- yl)-3,6-dimethyl-4-oxo- chromen-8- yl]ethoxy]pyridine-2- carboxamide | 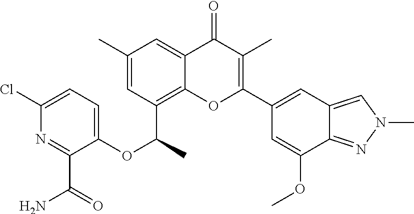 [SMILES: COc1cc(-c2oc3c([C@@H](C)Oc4ccc(Cl)nc4C(N)=O)cc(C)cc3c(=O)c2C)cc2cn(C)nc12] | 531 [M − H]⁻ |
| 45C | 6-Chloro-3-[(1R)-1-[2-(2,7- dimethylindazol-5-yl)-3,6- dimethyl-4-oxo-chromen-8- yl]ethoxy]pyridine-2- carboxamide | 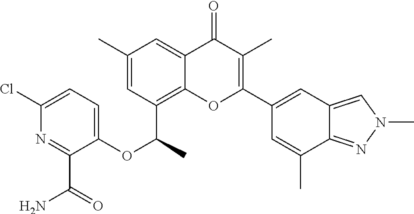 [SMILES: Cc1cc([C@@H](C)Oc2ccc(Cl)nc2C(N)=O)c2oc(-c3cc(C)c4nn(C)cc4c3)c(C)c(=O)c2c1] | 517 [M + H]⁺ |
| 46C | 6-Chloro-3-[(1R)-1-[2-(4- fluoro-2-methyl-indazol-5- yl)-3,6-dimethyl-4-oxo- chromen-8- yl]ethoxy]pyridine-2- carboxamide | 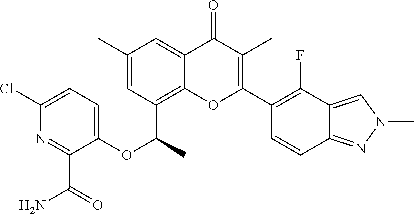 [SMILES: Cc1cc([C@@H](C)Oc2ccc(Cl)nc2C(N)=O)c2oc(-c3ccc4nn(C)cc4c3F)c(C)c(=O)c2c1] | 521 [M + H]⁺ |
| 47C | 6-Chloro-3-[(1R)-1-[2-(4,4- dimethyl-2-oxo-1H-3,1- benzoxazin-6-yl)-3,6- dimethyl-4-oxo-chromen-8- yl]ethoxy]pyridine-2- carboxamide | 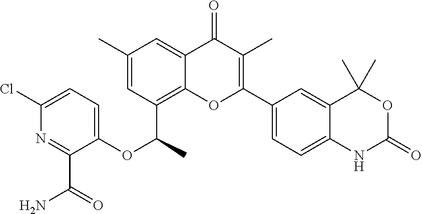 [SMILES: Cc1cc([C@@H](C)Oc2ccc(Cl)nc2C(N)=O)c2oc(-c3ccc4c(c3)C(C)(C)OC(=O)N4)c(C)c(=O)c2c1] | 546 [M − H]⁻ |
| 48C | 6-Chloro-3-[(1R)-1-[2-(3- fluoro-2-methyl-indazol-5- yl)-3,6-dimethyl-4-oxo- chromen-8- yl]ethoxy]pyridine-2- carboxamide | 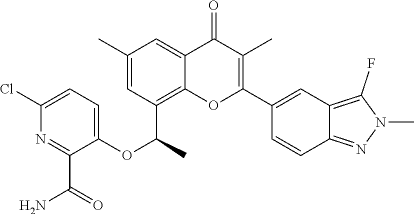 [SMILES: Cc1cc([C@@H](C)Oc2ccc(Cl)nc2C(N)=O)c2oc(-c3ccc4nn(C)c(F)c4c3)c(C)c(=O)c2c1] | 521 [M + H]⁺ |
| 49C | 6-Chloro-3-[(1R)-1-[2-(1,3- dimethylindazol-5-yl)-3,6- dimethyl-4-oxo-chromen-8- yl]ethoxy]pyridine-2- carboxamide | 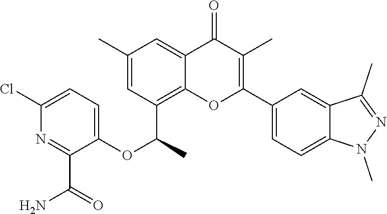 [SMILES: Cc1cc([C@@H](C)Oc2ccc(Cl)nc2C(N)=O)c2oc(-c3ccc4c(c3)c(C)nn4C)c(C)c(=O)c2c1] | 515 [M − H]⁻ |
| 50C | 6-Chloro-3-[(1R)-1-[2-(2,3- dimethylindazol-5-yl)-3,6- dimethyl-4-oxo-chromen-8- yl]ethoxy]pyridine-2- carboxamide | 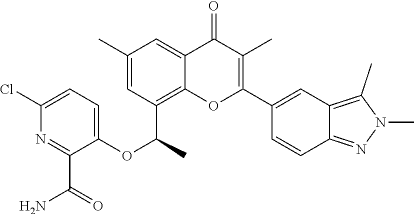 [SMILES: Cc1cc([C@@H](C)Oc2ccc(Cl)nc2C(N)=O)c2oc(-c3ccc4nn(C)c(C)c4c3)c(C)c(=O)c2c1] | 515 [M − H]⁻ |
| 51C | 6-Chloro-3-[(1R)-1-[3,6- dimethyl-2-(1-methylindazol- 3-yl)-4-oxo-chromen-8- yl]ethoxy]pyridine-2- carboxamide | 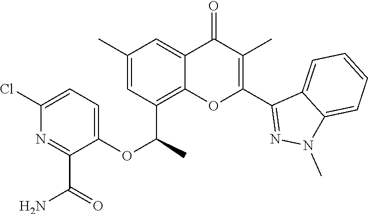 [SMILES: Cc1cc([C@@H](C)Oc2ccc(Cl)nc2C(N)=O)c2oc(-c3nn(C)c4ccccc34)c(C)c(=O)c2c1] | 503 [M + H]⁺ |
| 52C | 6-Chloro-3-[(1R)-1-[2-(7- cyano-2-methyl-indazol-5- yl)-3,6-dimethyl-4-oxo- chromen-8- yl]ethoxy]pyridine-2- carboxamide | 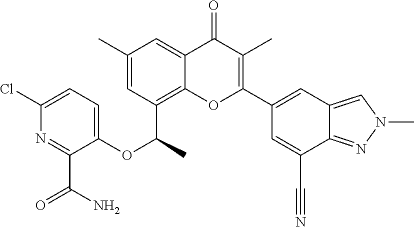 [SMILES: Cc1cc([C@@H](C)Oc2ccc(Cl)nc2C(N)=O)c2oc(-c3cc(C#N)c4nn(C)cc4c3)c(C)c(=O)c2c1] | 528 [M + H]⁺ |
| 53C | 6-Chloro-3-[(1R)-1-[3,6- dimethyl-2-(2-methyl-1,3- benzoxazol-6-yl)-4-oxo- chromen-8- yl]ethoxy]pyridine-2- carboxamide | 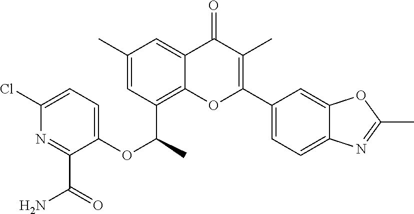 [SMILES: Cc1cc([C@@H](C)Oc2ccc(Cl)nc2C(N)=O)c2oc(-c3ccc4nc(C)oc4c3)c(C)c(=O)c2c1] | 504 [M + H]⁺ |
| 54C | 6-Chloro-3-[(1R)-1-[2-(3- fluoroimidazo[1,2-a]pyridin- 6-yl)-3,6-dimethyl-4-oxo- chromen-8- yl]ethoxy]pyridine-2- carboxamide | 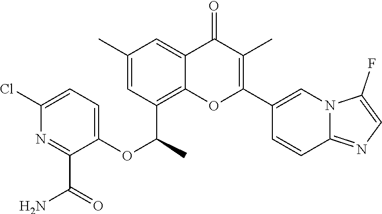 [SMILES: Cc1cc([C@@H](C)Oc2ccc(Cl)nc2C(N)=O)c2oc(-c3ccc4ncc(F)n4c3)c(C)c(=O)c2c1] | 507 [M + H]⁺ |
| 55C | 6-Chloro-3-[(1R)-1-(3,6- dimethyl-4-oxo-2- pyrazolo[1,5-a]pyridin-6-yl- chromen-8- yl)ethoxy]pyridine-2- carboxamide | 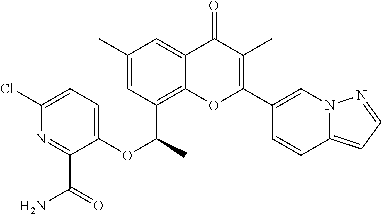 [SMILES: Cc1cc([C@@H](C)Oc2ccc(Cl)nc2C(N)=O)c2oc(-c3ccc4ccnn4c3)c(C)c(=O)c2c1] | 489 [M + H]⁺ |
| 56C | 6-Chloro-3-[(1R)-1-(2- furo[3,2-c]pyridin-2-yl-3,6- dimethyl-4-oxo-chromen-8- yl)ethoxy]pyridine-2- carboxamide | 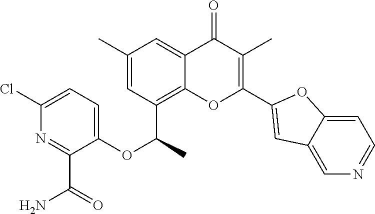 [SMILES: Cc1cc([C@@H](C)Oc2ccc(Cl)nc2C(N)=O)c2oc(-c3cc4cnccc4o3)c(C)c(=O)c2c1] | 490 [M + H]⁺ |
| 57C | 3-[(1R)-1-[2-(1,3- Benzoxazol-2-yl)-3,6- dimethyl-4-oxo-chromen-8- yl]ethoxy]-6-chloro-pyridine- 2-carboxamide | 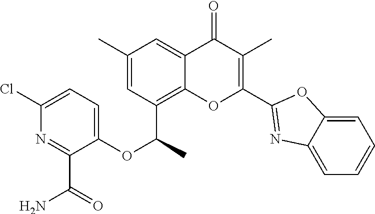 [SMILES: Cc1cc([C@@H](C)Oc2ccc(Cl)nc2C(N)=O)c2oc(-c3nc4ccccc4o3)c(C)c(=O)c2c1] | 490 [M + H]⁺ |
| 58C | 3-[(1R)-1-[2-(4- Acetamidophenyl)-3,6- dimethyl-4-oxo-chromen-8- yl]ethoxy]-6-chloro-pyridine- 2-carboxamide | 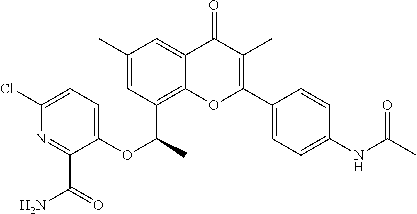 [SMILES: CC(=O)Nc1ccc(-c2oc3c([C@@H](C)Oc4ccc(Cl)nc4C(N)=O)cc(C)cc3c(=O)c2C)cc1] | 504 [M − H]⁻ |
| 59C | 3-[(1R)-1-[2-(2-Acetamido- 1,3-benzothiazol-6-yl)-3,6- dimethyl-4-oxo-chromen-8- yl]ethoxy]-6-chloro-pyridine- 2-carboxamide | 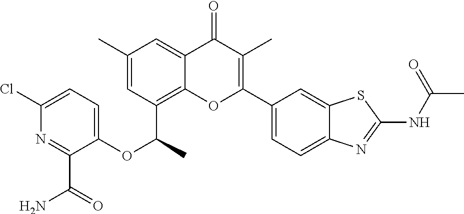 [SMILES: CC(=O)Nc1nc2ccc(-c3oc4c([C@@H](C)Oc5ccc(Cl)nc5C(N)=O)cc(C)cc4c(=O)c3C)cc2s1] | 563 [M + H]⁺ |
| 60C | 6-Chloro-3-[(1R)-1-[2-(2- methoxy-6-quinolyl)-3,6- dimethyl-4-oxo-chromen-8- yl]ethoxy]pyridine-2- carboxamide | 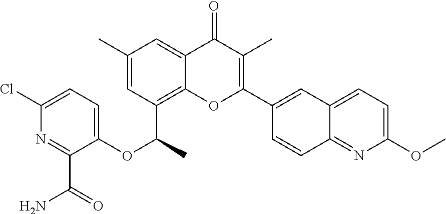 [SMILES: COc1ccc2cc(-c3oc4c([C@@H](C)Oc5ccc(Cl)nc5C(N)=O)cc(C)cc4c(=O)c3C)ccc2n1] | 530 [M + H]⁺ |
| 61C | 3-[(1R)-1-[2-(Benzofuran-5- yl)-3,6-dimethyl-4-oxo- chromen-8-yl]ethoxy]-6- chloro-pyridine-2- carboxamide | 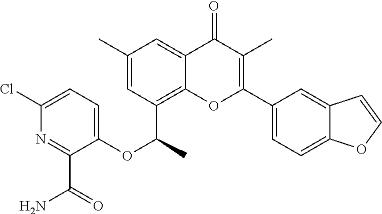 [SMILES: Cc1cc([C@@H](C)Oc2ccc(Cl)nc2C(N)=O)c2oc(-c3ccc4occc4c3)c(C)c(=O)c2c1] | 489 [M + H]⁺ |
| 62C | 6-Chloro-3-[(1R)-1-[3,6- dimethyl-2-(2-methyloxazol- 5-yl)-4-oxo-chromen-8- yl]ethoxy]pyridine-2- carboxamide | 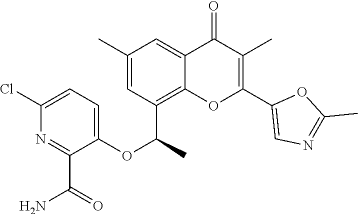 [SMILES: Cc1cc([C@@H](C)Oc2ccc(Cl)nc2C(N)=O)c2oc(-c3cnc(C)o3)c(C)c(=O)c2c1] | 452 [M − H]⁻ |
| 63C | 6-Chloro-3-[(1R)-1-[3,6- dimethyl-2-(2-methylthiazol- 5-yl)-4-oxo-chromen-8- yl]ethoxy]pyridine-2- carboxamide | 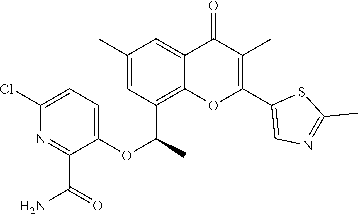 [SMILES: Cc1cc([C@@H](C)Oc2ccc(Cl)nc2C(N)=O)c2oc(-c3cnc(C)s3)c(C)c(=O)c2c1] | 468 [M − H]⁻ |
| 64C | 6-Chloro-3-[(1R)-1-[2-(2- cyanoimidazo[1,2-a]pyridin- 6-yl)-3,6-dimethyl-4-oxo- chromen-8- yl]ethoxy]pyridine-2- carboxamide | 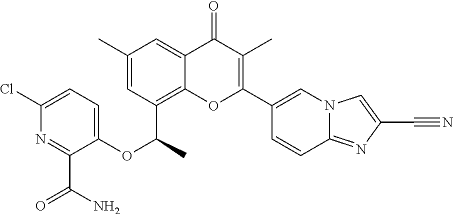 [SMILES: Cc1cc([C@@H](C)Oc2ccc(Cl)nc2C(N)=O)c2oc(-c3ccc4nc(C#N)cn4c3)c(C)c(=O)c2c1] | 514 [M + H]⁺ |
| 65C | 6-Chloro-3-[(1R)-1-[2-(3,3- dimethyl-2H-pyrazolo[5,1- b]oxazol-7-yl)-3,6-dimethyl- 4-oxo-chromen-8- yl]ethoxy]pyridine-2- carboxamide | 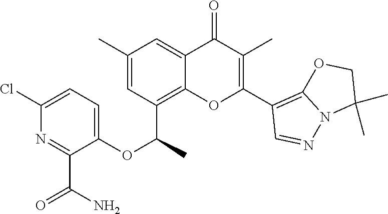 [SMILES: Cc1cc([C@@H](C)Oc2ccc(Cl)nc2C(N)=O)c2oc(-c3cnn4c3OCC4(C)C)c(C)c(=O)c2c1] | 509 [M + H]⁺ |
| 66C | 6-Chloro-3-[(1R)-1-[2-[2- (fluoromethyl)indazol-5-yl]- 3,6-dimethyl-4-oxo-chromen- 8-yl]ethoxy]pyridine-2- carboxamide | 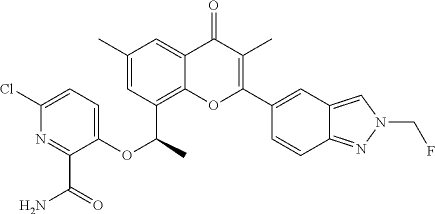 [SMILES: Cc1cc([C@@H](C)Oc2ccc(Cl)nc2C(N)=O)c2oc(-c3ccc4nn(CF)cc4c3)c(C)c(=O)c2c1] | 521 [M + H]⁺ |
| 67C | 6-Chloro-3-[(1R)-1-[2-(2,4- dimethylindazol-5-yl)-6- methyl-4-oxo-chromen-8- yl]ethoxy]pyridine-2- carboxamide | 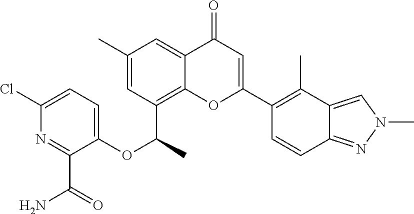 [SMILES: Cc1cc([C@@H](C)Oc2ccc(Cl)nc2C(N)=O)c2oc(-c3ccc4nn(C)cc4c3C)cc(=O)c2c1] | 501 [M − H]⁻ |
| 68C | 6-Chloro-3-[(1R)-1-[6- methyl-2-(2- methylimidazo[1,2-a]pyridin- 6-yl)-4-oxo-chromen-8- yl]ethoxy]pyridine-2- carboxamide | 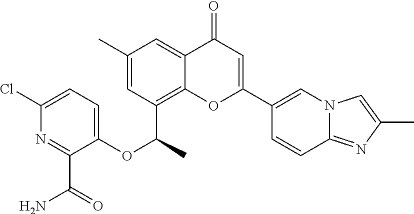 [SMILES: Cc1cc([C@@H](C)Oc2ccc(Cl)nc2C(N)=O)c2oc(-c3ccc4nc(C)cn4c3)cc(=O)c2c1] | 489 [M + H]⁺ |
| 69C | 6-Chloro-3-[(1R)-1-[3,6- dimethyl-2-(2-methyl-1,3- benzothiazol-6-yl)-4-oxo- chromen-8-yl]ethoxy]-N- methoxy-pyridine-2- carboxamide | 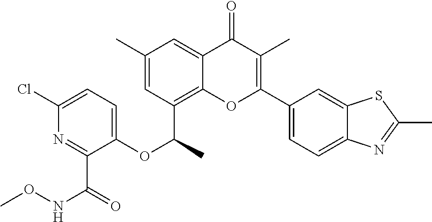 | 548 [M − H]⁻ |
| 70C | 6-Chloro-3-[(1R)-1-[3,6- dimethyl-2-(2- methylimidazo[1,2-a]pyridin- 6-yl)-4-oxo-chromen-8- yl]ethoxy]-N-methoxy- pyridine-2-carboxamide | 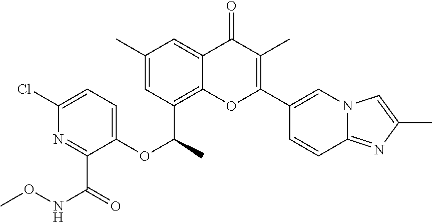 | 533 [M + H]⁺ |
| 71C | 5-[8-[(1R)-1-[(6-Chloro-2- methyl-3-pyridyl)oxy]ethyl]- 3,6-dimethyl-4-oxo-chromen- 2-yl]-1H-pyridin-2-one | 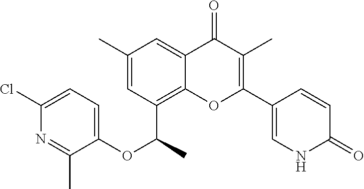 [SMILES: Cc1cc([C@@H](C)Oc2ccc(Cl)nc2C)c2oc(-c3ccc(=O)[nH]c3)c(C)c(=O)c2c1] | 437 [M + H]⁺ |
| 72C | 8-[(1R)-1-[(6-Chloro-2- methyl-3-pyridyl)oxy]ethyl]- 2-[1-(2- hydroxyethyl)pyrazol-4-yl]- 3,6-dimethyl-chromen-4-one | 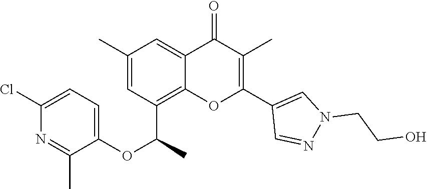 [SMILES: Cc1cc([C@@H](C)Oc2ccc(Cl)nc2C)c2oc(-c3cnn(CCO)c3)c(C)c(=O)c2c1] | 454 [M + H]⁺ |
| 73C | 6-Fluoro-3-[(1R)-1-(2- furo[3,2-b]pyridin-6-yl-3,6- dimethyl-4-oxo-chromen-8- yl)ethoxy]pyridine-2- sulfonamide | 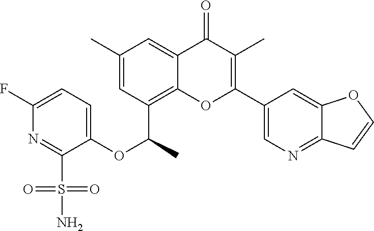 [SMILES: Cc1cc([C@@H](C)Oc2ccc(F)nc2S(N)(=O)=O)c2oc(-c3cnc4ccoc4c3)c(C)c(=O)c2c1] | 510 [M + H]⁺ |
| 74C | 3-[(1R)-1-(3,6-Dimethyl-4- oxo-2-phenyl-chromen-8- yl)ethoxy]-6-fluoro-pyridine- 2-sulfonamide | 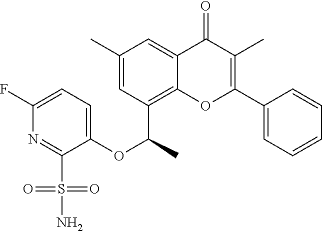 [SMILES: Cc1cc([C@@H](C)Oc2ccc(F)nc2S(N)(=O)=O)c2oc(-c3ccccc3)c(C)c(=O)c2c1] | 469 [M + H]⁺ |

### Example 20B and Example 21B: 6-Chloro-3-[1-[7-chloro-3,6-dimethyl-4-oxo-2-(3-pyridyl)chromen-8-yl]ethoxy]pyridine-2-carboxamide, Isomer 1 and Isomer 2

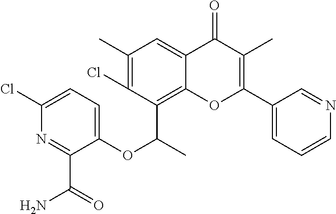 [SMILES: Cc1cc2c(=O)c(C)c(-c3cccnc3)oc2c(C(C)Oc2ccc(Cl)nc2C(N)=O)c1Cl]

A mixture of 6-chloro-3-[1-[7-chloro-3,6-dimethyl-4-oxo-2-(3-pyridyl)chromen-8-yl]ethoxy]pyridine-2-carbonitrile (0.070 g, 0.150 mmol) and hydrido(dimethylphosphinous acid-kP)[hydrogen bis(dimethylphosphinito-kP)]platinum(II) (0.013 g, 0.030 mmol) in EtOH (2 mL) and water (2 mL) was stirred at 50° C. for 12 h. The reaction mixture was concentrated under reduced pressure and the residue purified by reversed phase chromatography on C18 eluted with 0% to 100% ACN in water to give the racemic product (0.033 g, 45%) as a white solid. Chiral separation [Prep-Chiral-HPLC; Phenomenex Lux i-Amylose-3, 30×150 mm, 20-100% EtOH in heptane, 42.5 mL/min] was carried out to give the two title compounds. MS ES+ m/z 484 [M+H]⁺.

The following compounds in Table 28 were made in a similar way as described for 6-chloro-3-[1-[7-chloro-3,6-dimethyl-4-oxo-2-(3-pyridyl)chromen-8-yl]ethoxy]pyridine-2-carboxamide. Various methods were used to purify the compounds, which would be apparent to one skilled in the art.

TABLE 28

| Example | | | MS ES+ |
|---|---|---|---|
| # | Chemical Name | Structure | m/z |
| 22B | 6-Chloro-3-[(1R)-1-[2-(1H- indazol-5-yl)-3,6-dimethyl- 4-oxo-chromen-8- yl]ethoxy]pyridine-2- carboxamide | 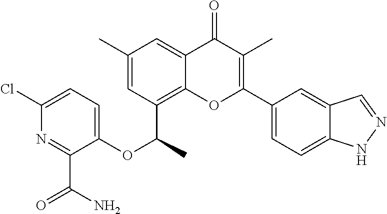 [SMILES: Cc1cc([C@@H](C)Oc2ccc(Cl)nc2C(N)=O)c2oc(-c3ccc4[nH]ncc4c3)c(C)c(=O)c2c1] | 487 [M − H]⁻ |
| 23B | 3-[(1R)-1-[3,6-Dimethyl-2- (1-methylpyrazol-4-yl)-4- oxo-chromen-8-yl]ethoxy]- 6-fluoro-pyridine-2- carboxamide | 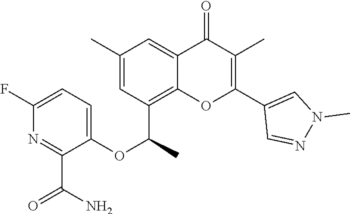 [SMILES: Cc1cc([C@@H](C)Oc2ccc(F)nc2C(N)=O)c2oc(-c3cnn(C)c3)c(C)c(=O)c2c1] | 435 [M − H]⁻ |
| 24B | 3-[(1R)-1-[3,6-Dimethyl-2- (2-methylindazol-5-yl)-4- oxo-chromen-8-yl]ethoxy]- 6-fluoro-pyridine-2- carboxamide | 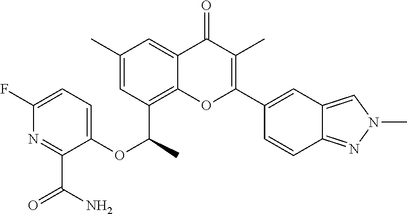 [SMILES: Cc1cc([C@@H](C)Oc2ccc(F)nc2C(N)=O)c2oc(-c3ccc4nn(C)cc4c3)c(C)c(=O)c2c1] | 485 [M − H]⁻ |
| 75C | 6-Chloro-3-[(1R)-1-[2-(2- ethylindazol-5-yl)-3,6- dimethyl-4-oxo-chromen-8- yl]ethoxy]pyridine-2- carboxamide | 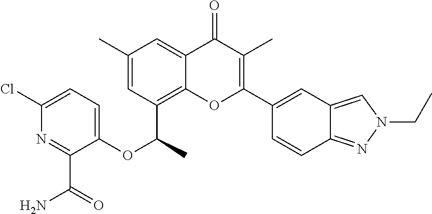 [SMILES: CCn1cc2cc(-c3oc4c([C@@H](C)Oc5ccc(Cl)nc5C(N)=O)cc(C)cc4c(=O)c3C)ccc2n1] | 517 [M + H]⁺ |
| 76C | 6-Chloro-3-[(1R)-1-[3,6- dimethyl-2-(2- methylpyrazol[1,5- a]pyridin-5-yl)-4-oxo- chromen-8- yl]ethoxy]pyridine-2- carboxamide | 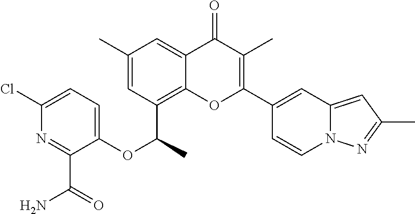 [SMILES: Cc1cc([C@@H](C)Oc2ccc(Cl)nc2C(N)=O)c2oc(-c3ccn4nc(C)cc4c3)c(C)c(=O)c2c1] | 501 [M − H]⁻ |
| 77C | 6-Chloro-3-[(1R)-1-[3,6- dimethyl-2-[2-(oxetan-3- yl)indazol-5-yl]-4-oxo- chromen-8- yl]ethoxy]pyridine-2- carboxamide | 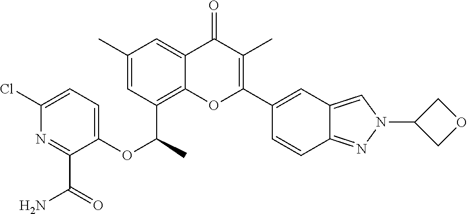 [SMILES: Cc1cc([C@@H](C)Oc2ccc(Cl)nc2C(N)=O)c2oc(-c3ccc4nn(C5COC5)cc4c3)c(C)c(=O)c2c1] | 545 [M + H]⁺ |
| 78C | 6-Chloro-3-[(1R)-1-(3,6- dimethyl-4-oxo-2-pyrazin-2- yl-chromen-8- yl)ethoxy]pyridine-2- carboxamide | 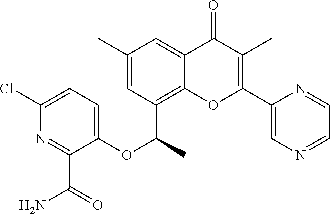 [SMILES: Cc1cc([C@@H](C)Oc2ccc(Cl)nc2C(N)=O)c2oc(-c3cnccn3)c(C)c(=O)c2c1] | 451 [M + H]⁺ |
| 79C | 6-Chloro-3-[(1R)-1-[2-(2- isopropoxypyrimidin-5-yl)- 3,6-dimethyl-4-oxo- chromen-8- yl]ethoxy]pyridine-2- carboxamide | 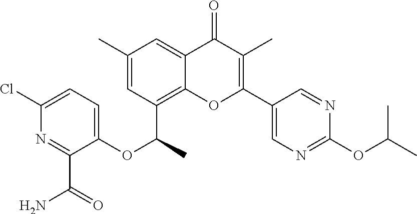 [SMILES: Cc1cc([C@@H](C)Oc2ccc(Cl)nc2C(N)=O)c2oc(-c3cnc(OC(C)C)nc3)c(C)c(=O)c2c1] | 509 [M + H]⁺ |
| 80C | 6-Chloro-3-[(1R)-1-(2- furo[3,2-b]pyridin-6-yl-3,6- dimethyl-4-oxo-chromen-8- yl)ethoxy]pyridine-2- carboxamide | 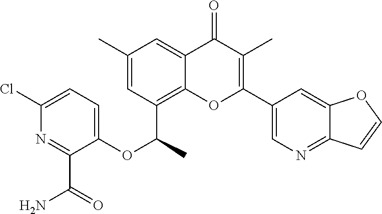 [SMILES: Cc1cc([C@@H](C)Oc2ccc(Cl)nc2C(N)=O)c2oc(-c3cnc4ccoc4c3)c(C)c(=O)c2c1] | 490 [M + H]⁺ |
| 81C | 6-Bromo-3-[(1R)-1-[3,6- dimethyl-2-(2- methylindazol-5-yl)-4-oxo- chromen-8- yl]ethoxy]pyridine-2- carboxamide | 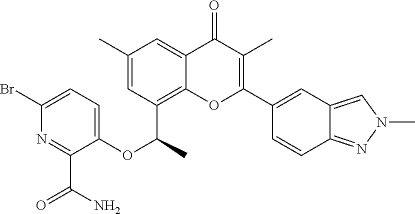 [SMILES: Cc1cc([C@@H](C)Oc2ccc(Br)nc2C(N)=O)c2oc(-c3ccc4nn(C)cc4c3)c(C)c(=O)c2c1] | 545 [M − H]⁻ |
| 82C | 2-[(1R)-1-[3,6-Dimethyl-2- (2-methylindazol-5-yl)-4- oxo-chromen-8-yl]ethoxy]- 6-fluoro-benzamide | 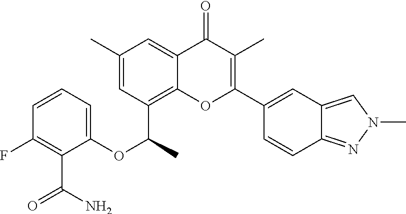 [SMILES: Cc1cc([C@@H](C)Oc2cccc(F)c2C(N)=O)c2oc(-c3ccc4nn(C)cc4c3)c(C)c(=O)c2c1] | 484 [M − H]⁻ |
| 83C | 6-Chloro-3-[(1R)-1-[2-(2- ethyl-1,3-benzothiazol-6-yl)- 3,6-dimethyl-4-oxo- chromen-8- yl]ethoxy]pyridine-2- carboxamide | 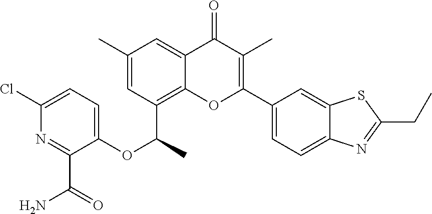 [SMILES: CCc1nc2ccc(-c3oc4c([C@@H](C)Oc5ccc(Cl)nc5C(N)=O)cc(C)cc4c(=O)c3C)cc2s1] | 534 [M + H]⁺ |
| 84C | 6-Chloro-3-[(1R)-1-[2-(2- ethyl-1,3-benzoxazol-6-yl)- 3,6-dimethyl-4-oxo- chromen-8- yl]ethoxy]pyridine-2- carboxamide | 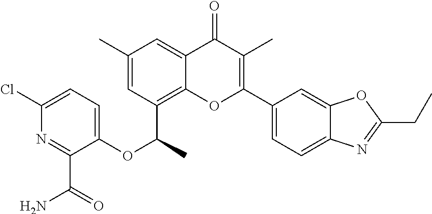 [SMILES: CCc1nc2ccc(-c3oc4c([C@@H](C)Oc5ccc(Cl)nc5C(N)=O)cc(C)cc4c(=O)c3C)cc2o1] | 518 [M + H]⁺ |

### Example 25B: 6-Chloro-3-[(1R)-1-[3-cyano-6-methyl-2-(1-methylpyrazol-4-yl)-4-oxo-chromen-8-yl]ethoxy]pyridine-2-sulfonamide

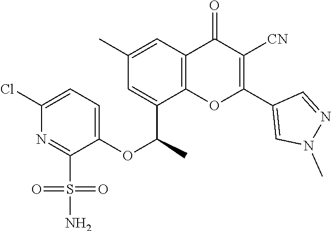 [SMILES: Cc1cc([C@@H](C)Oc2ccc(Cl)nc2S(N)(=O)=O)c2oc(-c3cnn(C)c3)c(C#N)c(=O)c2c1]

A 0° C. mixture of 8-[(1S)-1-hydroxyethyl]-6-methyl-2-(1-methylpyrazol-4-yl)-4-oxo-chromene-3-carbonitrile (0.573 g, 1.85 mmol), 6-chloro-3-hydroxy-pyridine-2-sulfonamide (1.16 g, 5.56 mmol), and triphenylphosphine (1.70 g, 6.48 mmol) in THE (22 mL) was treated with diisopropyl azodicarboxylate (1.31 g, 6.48 mmol). After 2 h, the reaction was concentrated under reduced pressure. The residue was purified three times by silica gel chromatography eluted with a gradient of 0 to 6% MeOH in DCM, then twice with 35 to 70% EtOAc in heptanes, to give the title compound (0.503 g, 53%). MS ES+ m/z 500 [M+H]⁺.

The following compounds in Table 29 were made in a similar way as described for 6-chloro-3-[(1R)-1-[3-cyano-6-methyl-2-(1-methylpyrazol-4-yl)-4-oxo-chromen-8-yl]ethoxy]pyridine-2-sulfonamide. Various methods were used to purify the compounds, which would be apparent to one skilled in the art.

TABLE 29

| Example | | | MS ES+ |
|---|---|---|---|
| # | Chemical Name | Structure | m/z |
| 85C | 6-Chloro-3-[(1R)-1-[2-(2,3- dihydropyrazolo[5,1- b]oxazol-7-yl)-3,6- dimethyl-4-oxo-chromen-8- yl]ethoxy]pyridine-2- sulfonamide | 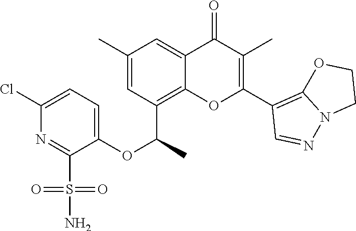 [SMILES: Cc1cc([C@@H](C)Oc2ccc(Cl)nc2S(N)(=O)=O)c2oc(-c3cnn4c3OCC4)c(C)c(=O)c2c1] | 517 [M + H]⁺ |
| 86C | 6-Chloro-3-[(1R)-1-[2-(6,8- dihydro-5H-imidazo[2,1- c][1,4]oxazin-3-yl)-3,6- dimethyl-4-oxo-chromen-8- yl]ethoxy]pyridine-2- sulfonamide | 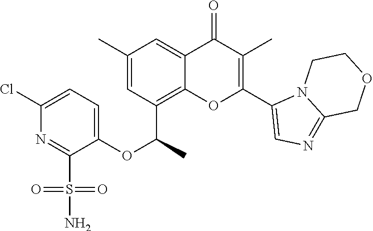 [SMILES: Cc1cc([C@@H](C)Oc2ccc(Cl)nc2S(N)(=O)=O)c2oc(-c3cnc4n3CCOC4)c(C)c(=O)c2c1] | 531 [M + H]⁺ |
| 87C | 6-Chloro-3-[(1R)-1-[3,6- dimethyl-2-(1,5- naphthyridin-3-yl)-4-oxo- chromen-8- yl]ethoxy]pyridine-2- sulfonamide | 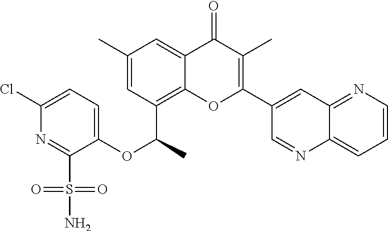 [SMILES: Cc1cc([C@@H](C)Oc2ccc(Cl)nc2S(N)(=O)=O)c2oc(-c3cnc4cccnc4c3)c(C)c(=O)c2c1] | 537 [M + H]⁺ |
| 88C | 6-Chloro-3-[(1R)-1-[2-[2- (2-hydroxyethyl)indazol-5- yl]-3,6-dimethyl-4-oxo- chromen-8- yl]ethoxy]pyridine-2- sulfonamide | 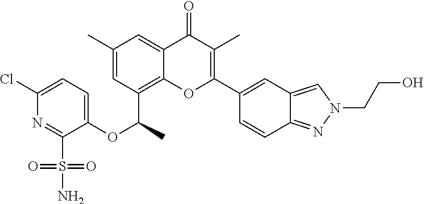 [SMILES: Cc1cc([C@@H](C)Oc2ccc(Cl)nc2S(N)(=O)=O)c2oc(-c3ccc4nn(CCO)cc4c3)c(C)c(=O)c2c1] | 569 [M + H]⁺ |
| 89C | 6-Chloro-3-[(1R)-1-[2-(6,8- dihydro-5H-imidazo[2,1- c][1,4]oxazin-2-yl)-3,6- dimethyl-4-oxo-chromen-8- yl]ethoxy]pyridine-2- sulfonamide | 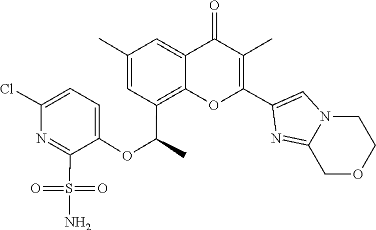 [SMILES: Cc1cc([C@@H](C)Oc2ccc(Cl)nc2S(N)(=O)=O)c2oc(-c3cn4c(n3)COCC4)c(C)c(=O)c2c1] | 531 [M + H]⁺ |
| 90C | 6-Chloro-3-[(1R)-1-[3,6- dimethyl-2-[1-(2- methylsulfonylethyl)pyrazol- 4-yl]-4-oxo-chromen-8- yl]ethoxy]pyridine-2- sulfonamide | 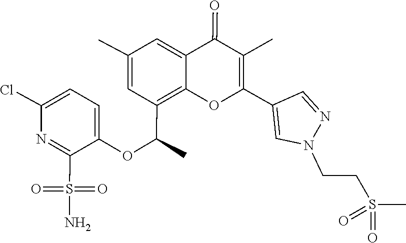 [SMILES: Cc1cc([C@@H](C)Oc2ccc(Cl)nc2S(N)(=O)=O)c2oc(-c3cnn(CCS(C)(=O)=O)c3)c(C)c(=O)c2c1] | 581 [M + H]⁺ |
| 91C | 6-Chloro-3-[(1R)-1-[3,6- dimethyl-2-(1- methylimidazol-4-yl)-4- oxo-chromen-8- yl]ethoxy]pyridine-2- sulfonamide | 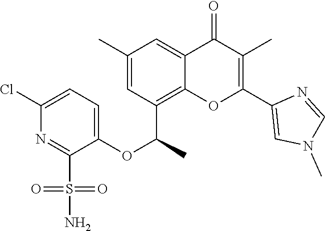 [SMILES: Cc1cc([C@@H](C)Oc2ccc(Cl)nc2S(N)(=O)=O)c2oc(-c3cn(C)cn3)c(C)c(=O)c2c1] | 489 [M + H]⁺ |
| 92C | 3-[(1R)-1-[3,6-Dimethyl-2- (1-methylpyrazol-4-yl)-4- oxo-chromen-8-yl]ethoxy]- 6-methoxy-pyridine-2- sulfonamide | 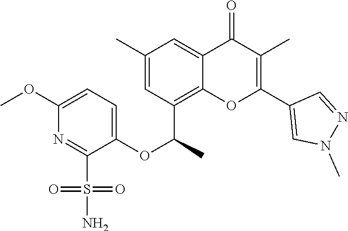 [SMILES: COc1ccc(O[C@H](C)c2cc(C)cc3c(=O)c(C)c(-c4cnn(C)c4)oc23)c(S(N)(=O)=O)n1] | 485 [M + H]⁺ |
| 93C | 3-[(1R)-1-[3,6-Dimethyl-2- (1-methylpyrazol-4-yl)-4- oxo-chromen-8- yl]ethoxy]pyridine-2- sulfonamide | 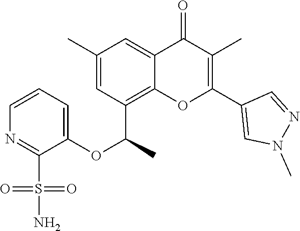 [SMILES: Cc1cc([C@@H](C)Oc2cccnc2S(N)(=O)=O)c2oc(-c3cnn(C)c3)c(C)c(=O)c2c1] | 455 [M + H]⁺ |
| 94C | 3-[(1R)-1-(3,6-Dimethyl-4- oxo-2-phenyl-chromen-8- yl)ethoxy]pyridine-2- sulfonamide | 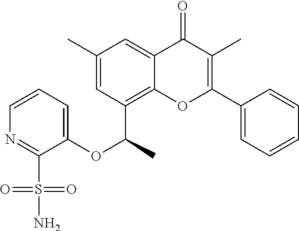 [SMILES: Cc1cc([C@@H](C)Oc2cccnc2S(N)(=O)=O)c2oc(-c3ccccc3)c(C)c(=O)c2c1] | 451 [M + H]⁺ |
| 95C | 3-[(1R)-1-[2-(2,3- Dihydropyrazolo[5,1- b]oxazol-7-yl)-3,6- dimethyl-4-oxo-chromen-8- yl]ethoxy]pyridine-2- sulfonamide | 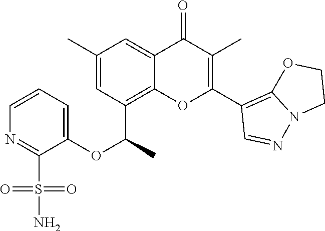 [SMILES: Cc1cc([C@@H](C)Oc2cccnc2S(N)(=O)=O)c2oc(-c3cnn4c3OCC4)c(C)c(=O)c2c1] | 483 [M + H]⁺ |
| 96C | 3-[(1R)-1-(2-Furo[3,2- b]pyridin-6-yl-3,6-dimethyl- 4-oxo-chromen-8- yl)ethoxy]pyridine-2- sulfonamide | 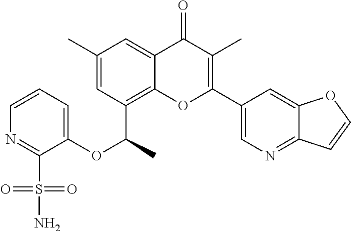 [SMILES: Cc1cc([C@@H](C)Oc2cccnc2S(N)(=O)=O)c2oc(-c3cnc4ccoc4c3)c(C)c(=O)c2c1] | 492 [M + H]⁺ |
| 97C | 3-[(1R)-1-(2-Cyclopropyl- 3,6-dimethyl-4-oxo- chromen-8- yl)ethoxy]pyridine-2- sulfonamide | 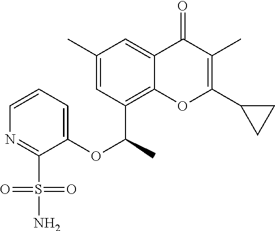 [SMILES: Cc1cc([C@@H](C)Oc2cccnc2S(N)(=O)=O)c2oc(C3CC3)c(C)c(=O)c2c1] | 415 [M + H]⁺ |
| 98C | 5-Chloro-2-[(1R)-1-(3,6- dimethyl-4-oxo-2-phenyl- chromen-8- yl)ethoxy]benzenesulfonamide | 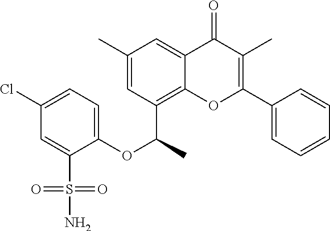 [SMILES: Cc1cc([C@@H](C)Oc2ccc(Cl)cc2S(N)(=O)=O)c2oc(-c3ccccc3)c(C)c(=O)c2c1] | 484 [M + H]⁺ |
| 99C | 5-Chloro-2-[(1R)-1-[3,6- dimethyl-2-(1- methylpyrazol-4-yl)-4-oxo- chromen-8- yl]ethoxy]benzenesulfonamide | 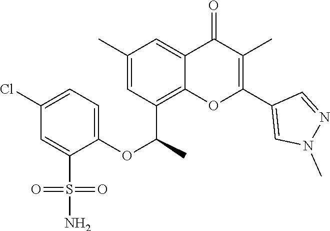 [SMILES: Cc1cc([C@@H](C)Oc2ccc(Cl)cc2S(N)(=O)=O)c2oc(-c3cnn(C)c3)c(C)c(=O)c2c1] | 488 [M + H]⁺ |
| 100C | 5-Chloro-2-[(1R)-1-(2- furo[3,2-b]pyridin-6-yl-3,6- dimethyl-4-oxo-chromen-8- yl)ethoxy]benzenesulfonamide | 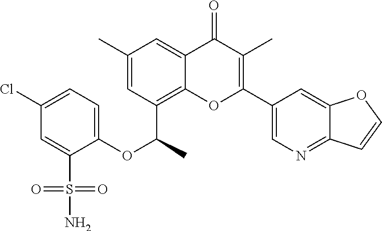 [SMILES: Cc1cc([C@@H](C)Oc2ccc(Cl)cc2S(N)(=O)=O)c2oc(-c3cnc4ccoc4c3)c(C)c(=O)c2c1] | 525 [M + H]⁺ |
| 101C | 5-Chloro-2-[(1R)-1-(2- cyclopropyl-3,6-dimethyl-4- oxo-chromen-8- yl)ethoxy]benzenesulfonamide | 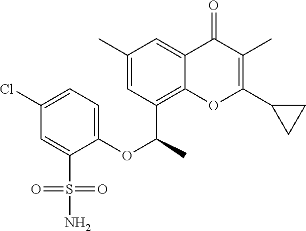 [SMILES: Cc1cc([C@@H](C)Oc2ccc(Cl)cc2S(N)(=O)=O)c2oc(C3CC3)c(C)c(=O)c2c1] | 44/8 [M + H]⁺ |
| 102C | 2-[(1R)-1-(3,6-Dimethyl-4- oxo-2-phenyl-chromen-8- yl)ethoxy]benzenesulfonamide | 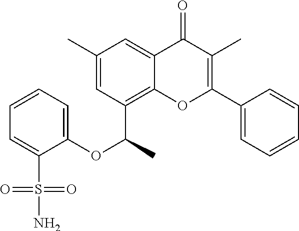 [SMILES: Cc1cc([C@@H](C)Oc2ccccc2S(N)(=O)=O)c2oc(-c3ccccc3)c(C)c(=O)c2c1] | 450 [M + H]⁺ |
| 103C | 2-[(1R)-1-[3,6-Dimethyl-2- (1-methylpyrazol-4-yl)-4- oxo-chromen-8- yl]ethoxy]benzenesulfonamide | 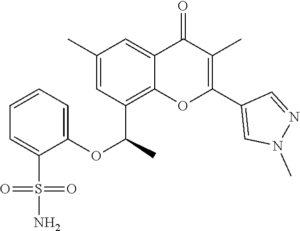 [SMILES: Cc1cc([C@@H](C)Oc2ccccc2S(N)(=O)=O)c2oc(-c3cnn(C)c3)c(C)c(=O)c2c1] | 454 [M + H]⁺ |
| 104C | 2-[(1R)-1-(2-Cyclopropyl- 3,6-dimethyl-4-oxo- chromen-8- yl)ethoxy]benzenesulfonamide | 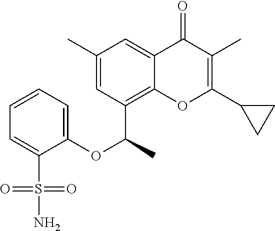 [SMILES: Cc1cc([C@@H](C)Oc2ccccc2S(N)(=O)=O)c2oc(C3CC3)c(C)c(=O)c2c1] | 414 [M + H]⁺ |
| 105C | 2-[(1R)-1-[3,6-Dimethyl-2- (1-methylpyrazol-4-yl)-4- oxo-chromen-8-yl]ethoxy]- 5-fluoro- benzenesulfonamide | 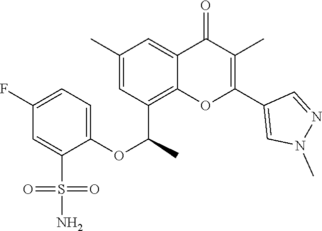 [SMILES: Cc1cc([C@@H](C)Oc2ccc(F)cc2S(N)(=O)=O)c2oc(-c3cnn(C)c3)c(C)c(=O)c2c1] | 472 [M + H]⁺ |
| 106C | 5-Fluoro-2-[(1R)-1-(2- furo[3,2-b]pyridin-6-yl-3,6- dimethyl-4-oxo-chromen-8- yl)ethoxy]benzenesulfonamide | 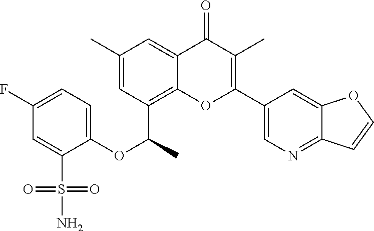 [SMILES: Cc1cc([C@@H](C)Oc2ccc(F)cc2S(N)(=O)=O)c2oc(-c3cnc4ccoc4c3)c(C)c(=O)c2c1] | 509 [M + H]⁺ |
| 107C | 2-[(1R)-1-(2-Cyclopropyl- 3,6-dimethyl-4-oxo- chromen-8-yl)ethoxy]-5- fluoro-benzenesulfonamide | 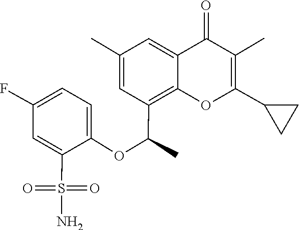 [SMILES: Cc1cc([C@@H](C)Oc2ccc(F)cc2S(N)(=O)=O)c2oc(C3CC3)c(C)c(=O)c2c1] | 432 [M + H]⁺ |
| 108C | 5-Cyano-2-[(1R)-1-[3,6- dimethyl-2-(2- methylindazol-5-yl)-4-oxo- chromen-8- yl]ethoxy]benzamide | 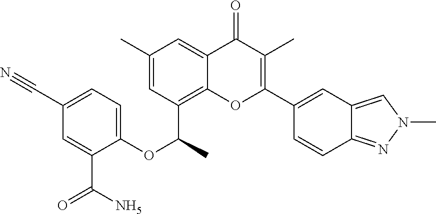 | 491 [M − H]⁻ |
| 109C | 3-[(1R)-1-(2-Cyclopropyl- 3,6-dimethyl-4-oxo- chromen-8-yl)ethoxy]-6- fluoro-pyridine-2- sulfonamide | 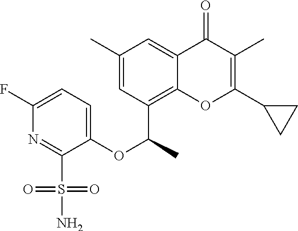 [SMILES: Cc1cc([C@@H](C)Oc2ccc(F)nc2S(N)(=O)=O)c2oc(C3CC3)c(C)c(=O)c2c1] | 433 [M + H]⁺ |
| 110C | 6-[(1R)-1-[3,6-Dimethyl-2- (1-methylpyrazol-4-yl)-4- oxo-chromen-8-yl]ethoxy]- 2,3-difluoro- benzenesulfonamide | 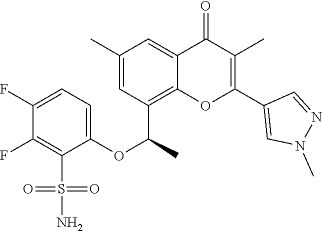 [SMILES: Cc1cc([C@@H](C)Oc2ccc(F)c(F)c2S(N)(=O)=O)c2oc(-c3cnn(C)c3)c(C)c(=O)c2c1] | 490 [M + H]⁺ |
| 111C | 6-Chloro-3-[rel-(1R)-1-[3- chloro-6-methyl-2-(1- methylpyrazol-4-yl)-4-oxo- chromen-8- yl]ethoxy]pyridine-2- sulfonamide | 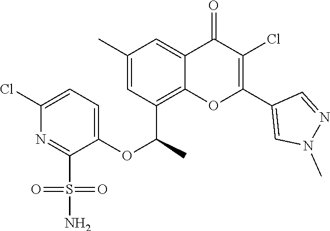 [SMILES: Cc1cc([C@@H](C)Oc2ccc(Cl)nc2S(N)(=O)=O)c2oc(-c3cnn(C)c3)c(Cl)c(=O)c2c1] | 509 [M + H]⁺ |
| 112C ^{A} | 6-Chloro-3-[(1R)-1-[3,6- dimethyl-2-(5- oxaspiro[2.3]hexan-2-yl)-4- oxo-chromen-8- yl]ethoxy]pyridine-2- sulfonamide, Isomer 1 | 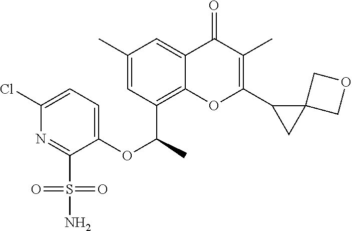 [SMILES: Cc1cc([C@@H](C)Oc2ccc(Cl)nc2S(N)(=O)=O)c2oc(C3CC34COC4)c(C)c(=O)c2c1] | 491 [M + H]⁺ |
| 113C ^{A} | 6-Chloro-3-[(1R)-1-[3,6- dimethyl-2-(5- oxaspiro[2.3]hexan-2-yl)-4- oxo-chromen-8- yl]ethoxy]pyridine-2- sulfonamide, Isomer 2 | 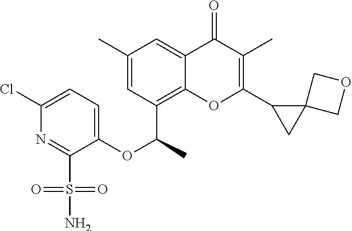 [SMILES: Cc1cc([C@@H](C)Oc2ccc(Cl)nc2S(N)(=O)=O)c2oc(C3CC34COC4)c(C)c(=O)c2c1] | 491 [M + H]⁺ |
| 114C | 6-Chloro-3-[(1R)-1-[3,6- dimethyl-2-(oxetan-3-yl)-4- oxo-chromen-8- yl]ethoxy]pyridine-2- sulfonamide | 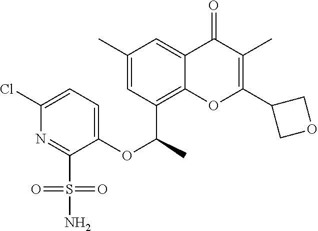 [SMILES: Cc1cc([C@@H](C)Oc2ccc(Cl)nc2S(N)(=O)=O)c2oc(C3COC3)c(C)c(=O)c2c1] | 465 [M + H]⁺ |

^{A} Reversed phase chromatography (C18) eluted with a gradient of 0 to 100% ACN in H₂O + 0.1% formic acid

### Example 115C: 6-Chloro-3-[(1R)-1-[3,6-dimethyl-4-oxo-2-(1H-pyrazol-4-yl)chromen-8-yl]ethoxy]pyridine-2-sulfonamide

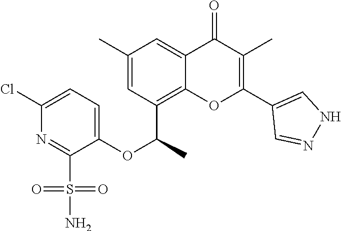 [SMILES: Cc1cc([C@@H](C)Oc2ccc(Cl)nc2S(N)(=O)=O)c2oc(-c3cn[nH]c3)c(C)c(=O)c2c1]

To a 0° C. mixture of 8-[(1R)-1-hydroxyethyl]-3,6-dimethyl-2-(1-tetrahydropyran-2-ylpyrazol-4-yl)chromen-4-one (0.150 g, 0.407 mmol) in THF (10 mL) was added sodium hydride (60%; 0.162 g, 4.07 mmol) in portions under nitrogen. The mixture was stirred at rt for 1 h, then 6-chloro-3-fluoro-pyridine-2-sulfonamide (0.171 g, 0.814 mmol) was added. The reaction mixture was stirred overnight at 50° C., cooled to 0° C., quenched with ice/water, and diluted with water (120 mL). The resulting mixture was extracted with EtOAc (3×10⁰ mL). The combined organic layers were washed with saturated aqueous sodium chloride (80 mL), dried over anhydrous sodium sulfate, and filtered. The filtrate was concentrated under reduced pressure to give crude 6-chloro-3-[(1R)-1-[3,6-dimethyl-4-oxo-2-(1-tetrahydropyran-2-ylpyrazol-4-yl)chromen-8-yl]ethoxy]pyridine-2-sulfonamide as a white solid. MS ES+ m/z 559 [M+H]⁺.

A solution of 6-chloro-3-[(1R)-1-[3,6-dimethyl-4-oxo-2-(1-tetrahydropyran-2-ylpyrazol-4-yl)chromen-8-yl]ethoxy]pyridine-2-sulfonamide (0.400 g, 0.716 mmol) and TFA (0.8 mL) in MeOH (4 mL) was stirred for 1 h at rt under nitrogen. The resulting mixture was diluted with water (100 mL) and basified to pH ˜8 with ammonium hydroxide. The resulting mixture was extracted with EtOAc (3×10⁰ mL). The combined organic layers were washed with saturated aqueous sodium chloride (60 mL), dried over anhydrous sodium sulfate, and filtered. The filtrate was concentrated under reduced pressure and the residue was purified by reversed phase chromatography eluted with 18% to 43% ACN in 10 mM aqueous NH₄HCO₃ with 0.05% ammonium hydroxide to give the title compound (0.044 g, 23%) as a white solid. MS ES+ m/z 475 [M+H]⁺.

The following compounds in Table 30 were made in a similar way as described for 6-chloro-3-[(1R)-1-[3,6-dimethyl-4-oxo-2-(1H-pyrazol-4-yl)chromen-8-yl]ethoxy]pyridine-2-sulfonamide. The deprotection step was not always carried out, depending on the intermediate used. Various methods were used to purify the compounds, which would be apparent to one skilled in the art.

TABLE 30

| | | | MS ES+ |
|---|---|---|---|
| Example # | Chemical Name | Structure | m/z |
| 116C | 6-Chloro-3-[(1R)-1-[3,6- dimethyl-2-[1-(2- oxabicyclo[2.1.1]hexan-4- ylmethyl)pyrazol-4-yl]-4- oxo-chromen-8- yl]ethoxy]pyridine-2- sulfonamide | 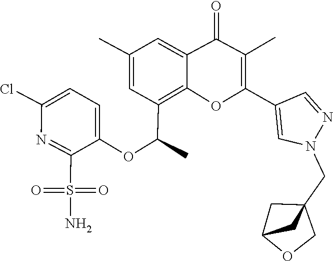 [SMILES: Cc1cc([C@@H](C)Oc2ccc(Cl)nc2S(N)(=O)=O)c2oc(-c3cnn(C[C@]45CO[C@H](C4)C5)c3)c(C)c(=O)c2c1] | 571 [M + H]⁺ |
| 117C | 6-Chloro-3-[(1R)-1-[2-[1- (2-methoxyethyl)pyrazol- 4-yl]-3,6-dimethyl-4-oxo- chromen-8- yl]ethoxy]pyridine-2- sulfonamide | 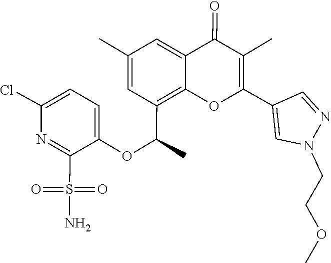 [SMILES: COCCn1cc(-c2oc3c([C@@H](C)Oc4ccc(Cl)nc4S(N)(=O)=O)cc(C)cc3c(=O)c2C)cn1] | 533 [M + H]⁺ |
| 118C | 6-Chloro-3-[(1R)-1-[3,6- dimethyl-2-[1-(2- oxabicyclo[2.1.1]hexan-1- ylmethyl)pyrazol-4-yl]-4- oxo-chromen-8- yl]ethoxy]pyridine-2- sulfonamide | 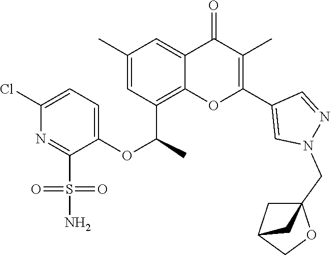 [SMILES: Cc1cc([C@@H](C)Oc2ccc(Cl)nc2S(N)(=O)=O)c2oc(-c3cnn(C[C@]45C[C@H](CO4)C5)c3)c(C)c(=O)c2c1] | 571 [M + H]⁺ |
| 119C | 6-Chloro-3-[(1R)-1-[2-[1- (3,3- difluoropropyl)pyrazol-4- yl]-3,6-dimethyl-4-oxo- chromen-8- yl]ethoxy]pyridine-2- sulfonamide | 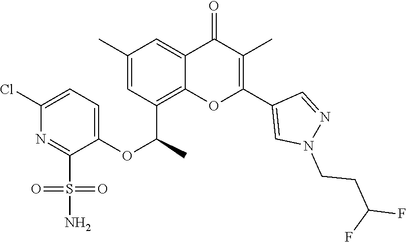 [SMILES: Cc1cc([C@@H](C)Oc2ccc(Cl)nc2S(N)(=O)=O)c2oc(-c3cnn(CCC(F)F)c3)c(C)c(=O)c2c1] | 553 [M + H]⁺ |
| 120C | 6-Chloro-3-[(1R)-1-[3,6- dimethyl-2-(2- methylimidazo[1,2- a]pyridin-6-yl)-4-oxo- chromen-8- yl]ethoxy]pyridine-2- sulfonamide | 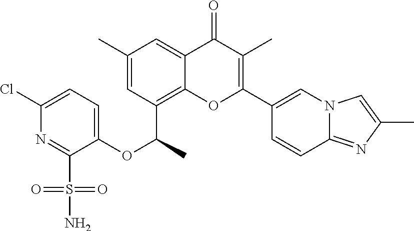 [SMILES: Cc1cc([C@@H](C)Oc2ccc(Cl)nc2S(N)(=O)=O)c2oc(-c3ccc4nc(C)cn4c3)c(C)c(=O)c2c1] | 539 [M + H]⁺ |
| 121C | 6-Chloro-3-[(1R)-1-[2-[1- (2-hydroxy-2-methyl- propyl)pyrazol-4-yl]-3,6- dimethyl-4-oxo-chromen- 8-yl]ethoxy]-N-methoxy- pyridine-2-sulfonamide | 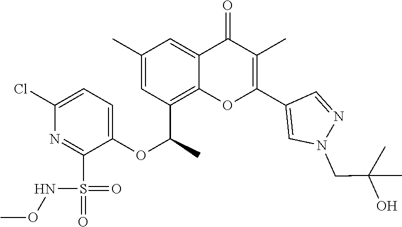 [SMILES: CONS(=O)(=O)c1nc(Cl)ccc1O[C@H](C)c1cc(C)cc2c(=O)c(C)c(-c3cnn(CC(C)(C)O)c3)oc12] | 577 [M + H]⁺ |
| 122C | 3-[(1R)-1-(3,6-Dimethyl-4- oxo-2-phenyl-chromen-8- yl)ethoxy]-6-methyl- pyridine-2-sulfonamide | 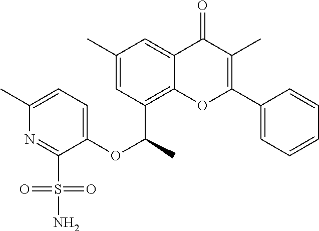 [SMILES: Cc1cc([C@@H](C)Oc2ccc(C)nc2S(N)(=O)=O)c2oc(-c3ccccc3)c(C)c(=O)c2c1] | 465 [M + H]⁺ |
| 123C | 3-[(1R)-1-[3,6-Dimethyl-2- (1-methylpyrazol-4-yl)-4- oxo-chromen-8-yl]ethoxy]- 6-methyl-pyridine-2- sulfonamide | 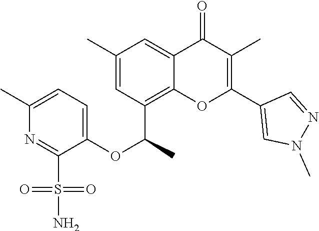 [SMILES: Cc1cc([C@@H](C)Oc2ccc(C)nc2S(N)(=O)=O)c2oc(-c3cnn(C)c3)c(C)c(=O)c2c1] | 469 [M + H]⁺ |

### Example 124C: 6-Chloro-3-[(1R)-1-[2-[1-[(1-cyanocyclopropyl)methyl]pyrazol-4-yl]-3,6-dimethyl-4-oxo-chromen-8-yl]ethoxy]pyri dine-2-sulfonamide

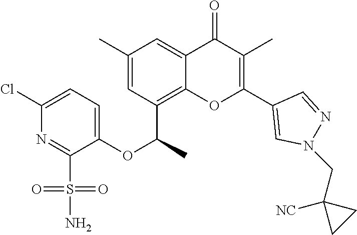 [SMILES: Cc1cc([C@@H](C)Oc2ccc(Cl)nc2S(N)(=O)=O)c2oc(-c3cnn(CC4(C#N)CC4)c3)c(C)c(=O)c2c1]

A mixture of 6-chloro-3-[(1R)-1-[3,6-dimethyl-4-oxo-2-(1H-pyrazol-4-yl)chromen-8-yl]ethoxy]pyridine-2-sulfonamide (0.040 g, 0.084 mmol), 1-bromomethyl-cyclopropanecarbonitrile (0.0081 g, 0.0051 mmol), and cesium carbonate (0.069 g, 0.21 mmol) in N,N-dimethylacetamide (2 mL) was stirred at 45° C. for 8 h. The crude reaction mixture was purified by reversed phase chromatography eluted with a gradient of 10 to 100% ACN in water (with formic acid). Fractions containing the desired product were combined, concentrated under reduced pressure, and lyophilized to give the title compound (0.004 g, 7%) as a white solid. MS ES+ m/z 554 [M+H]⁺.

### Examples 125C and 126C: 6-Chloro-3-[(1R)-1-[2-[2-(hydroxymethyl)cyclopropyl]-3,6-dimethyl-4-oxo-chromen-8-yl]ethoxy]pyridine-2-sulfonamide, Isomers 1 and 2

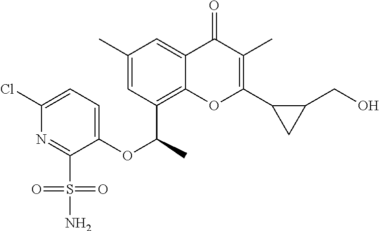 [SMILES: Cc1cc([C@@H](C)Oc2ccc(Cl)nc2S(N)(=O)=O)c2oc(C3CC3CO)c(C)c(=O)c2c1]

To a 0° C. solution of 2-[8-[(1R)-1-[(6-chloro-2-sulfamoyl-3-pyridyl)oxy]ethyl]-3,6-dimethyl-4-oxo-chromen-2-yl]cyclopropanecarboxylic acid (0.210 g, 0.426 mmol) in THE (3 mL) was added borane-THF complex (0.9 M, 1.18 mL, 1.07 mmol). The mixture was stirred at 0° C. for 1 h, then additional borane-THF complex (0.9 M, 0.23 mL, 0.21 mmol) was added. The mixture was allowed to warm to rt and stirred for 1 h. The reaction mixture was concentrated under reduced pressure and the residue was purified by reversed phase chromatography eluted with a gradient of 0 to 100% ACN in H₂O+0.1% formic acid. Fractions containing the desired products were separately combined, washed with saturated aqueous sodium chloride, and extracted with IPA:CHCl₃ (1:3).

The combined extracts were dried over MgSO₄, filtered, and concentrated under reduced pressure to give the title compounds (Isomer 1, 0.008 g, 4%; Isomer 2, 0.011 g, 5%). MS ES+ m/z 479 [M+H]*, for both.

### Example 127C: 6-Chloro-3-[(1R)-1-[2-[1-(2-hydroxyethyl)cyclopropyl]-3,6-dimethyl-4-oxo-chromen-8-yl]ethoxy]pyridine-2-sulfonamide

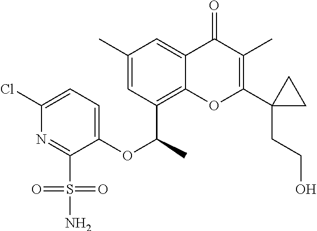 [SMILES: Cc1cc([C@@H](C)Oc2ccc(Cl)nc2S(N)(=O)=O)c2oc(C3(CCO)CC3)c(C)c(=O)c2c1]

2-[1-[8-[(1R)-1-[(6-Chloro-2-sulfamoyl-3-pyridyl)oxy]ethyl]-3,6-dimethyl-4-oxo-chromen-2-yl]cyclopropyl]acetic acid was used in a manner analogous to the method of preparation of Examples 125C and 126C to afford the title compound (0.063 g, 18%). MS ES+ m/z 493 [M+H]⁺.

### Example 128C: 6-Chloro-3-[(1R)-1-[2-[1-[(1-hydroxycyclopropyl)methyl]pyrazol-4-yl]-3,6-dimethyl-4-oxo-chromen-8-yl]ethoxy]pyridine-2-sulfonamide

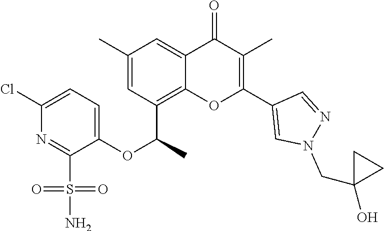 [SMILES: Cc1cc([C@@H](C)Oc2ccc(Cl)nc2S(N)(=O)=O)c2oc(-c3cnn(CC4(O)CC4)c3)c(C)c(=O)c2c1]

To a solution of 6-chloro-3-[(1R)-1-[3,6-dimethyl-4-oxo-2-[1-[(1-tetrahydropyran-2-yloxycyclopropyl)methyl]pyrazol-4-yl]chromen-8-yl]ethoxy]pyridine-2-sulfonamide (0.098 g, 0.16 mmol) in THE (2 mL) was added HCl (10% aqueous; 0.70 mL). The reaction mixture was heated at 50° C. for 1 h, then concentrated under reduced pressure. The residue was purified by reversed phase chromatography eluted with a gradient of 0 to 100% ACN in H₂O+0.1% formic acid. Fractions containing the desired product were combined, washed with saturated aqueous sodium chloride, and extracted with IPA:CHCl₃ (1:3). The combined extracts were dried over MgSO₄, filtered, and concentrated under reduced pressure to give the title compound (0.032 g, 38%) as a white solid. MS ES+ m/z 545 [M+H]⁺.

### Example 129C: 6-Chloro-3-[(1R)-1-[2-[2-(2-hydroxyethyl)indazol-5-yl]-3,6-dimethyl-4-oxo-chromen-8-yl]ethoxy]pyridine-2-carboxamide

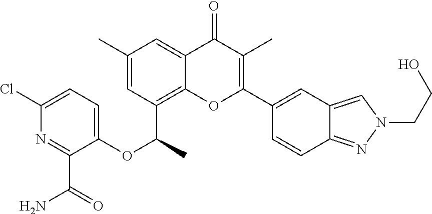 [SMILES: Cc1cc([C@@H](C)Oc2ccc(Cl)nc2C(N)=O)c2oc(-c3ccc4nn(CCO)cc4c3)c(C)c(=O)c2c1]

To a solution of 3-[(1R)-1-[2-[2-[2-[tert-butyl(dimethyl)silyl]oxyethyl]indazol-5-yl]-3,6-dimethyl-4-oxo-chromen-8-yl]ethoxy]-6-chloro-pyridine-2-carboxamide (60%; 0.228 g, 0.211 mmol) in THE (5 mL) was added tetra-n-butylammonium fluoride (1M in THF, 0.350 mL, 0.350 mmol) dropwise. The reaction was stirred at rt for 1 h, then quenched with saturated aqueous ammonium chloride (5 mL). The resulting mixture was extracted with DCM (3×). The combined organic layers were dried over anhydrous sodium sulfate, filtered, and concentrated under reduced pressure. The residue was purified by reversed phase chromatography on C18 eluted with 20% to 100% ACN in 10 mM aqueous NH₄HCO₃ with 5% MeOH. Fractions containing the desired product were combined, concentrated under reduced pressure, and lyophilized to give crude product. This material was dissolved in DCM (5 mL) and washed with saturated aqueous ammonium chloride (3×) and water (1×). The organic layer was dried over anhydrous sodium sulfate, filtered, and dried under a stream of nitrogen to give the title compound (0.061 g, 54%) as a white solid. MS ES+ m z 531 [M−H]⁻.

The following compounds in Table 31 were made in a similar way as described for 6-chloro-3-[(1R)-1-[2-[1-[(1-hydroxycyclopropyl)methyl]pyrazol-4-yl]-3,6-dimethyl-4-oxo-chromen-8-yl]ethoxy]pyridine-2-sulfonamide and 6-chloro-3-[(1R)-1-[2-[2-(2-hydroxyethyl)indazol-5-yl]-3,6-dimethyl-4-oxo-chromen-8-yl]ethoxy]pyridine-2-carboxamide. Various methods were used to purify the compounds, which would be apparent to one skilled in the art.

TABLE 31

| | | | MS ES+ |
|---|---|---|---|
| Example # | Chemical Name | Structure | m/z |
| 130C | 6-Chloro-3-[(1R)-1-[2-[2-[(1- hydroxycyclopropyl)methyl] indazol-5-yl]-3,6-dimethyl-4-oxo- chromen-8-yl]ethoxy]pyridine- 2-carboxamide | 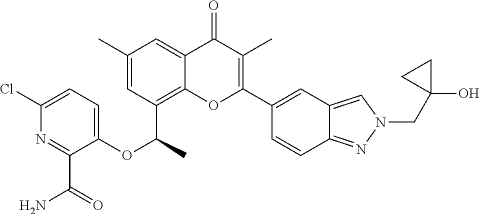 [SMILES: Cc1cc([C@@H](C)Oc2ccc(Cl)nc2C(N)=O)c2oc(-c3ccc4nn(CC5(O)CC5)cc4c3)c(C)c(=O)c2c1] | 557 [M − H]⁻ |
| 131C | 6-Chloro-3-[(1R)-1-[2-[2-(2- hydroxyethyl)imidazo[1,2- a]pyridin-6-yl]-3,6-dimethyl-4- oxo-chromen-8- yl]ethoxy]pyridine-2- carboxamide | 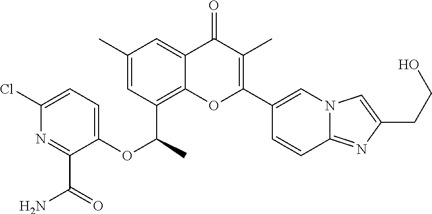 [SMILES: Cc1cc([C@@H](C)Oc2ccc(Cl)nc2C(N)=O)c2oc(-c3ccc4nc(CCO)cn4c3)c(C)c(=O)c2c1] | 533 [M + H]⁺ |
| 132C | 6-Chloro-3-[(1R)-1-[2-[2-(2- hydroxyethyl)-1,3- benzothiazol-6-yl]-3,6- dimethyl-4-oxo-chromen-8- yl]ethoxy]pyridine-2- carboxamide | 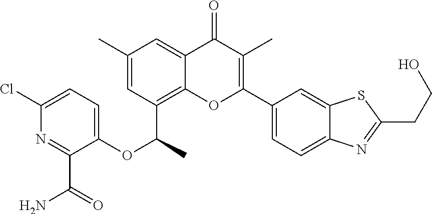 [SMILES: Cc1cc([C@@H](C)Oc2ccc(Cl)nc2C(N)=O)c2oc(-c3ccc4nc(CCO)sc4c3)c(C)c(=O)c2c1] | 572 [M + Na]⁺ |
| 133C | 6-Chloro-3-[(1R)-1-[2-[2-(3- hydroxycyclobutyl)indazol-5- yl]-3,6-dimethyl-4-oxo- chromen-8-yl]ethoxy]pyridine- 2-carboxamide | 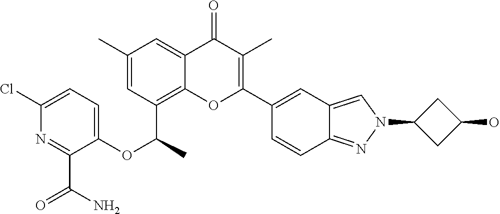 | 559 [M + H]⁺ |
| 134C | 6-Chloro-3-[(1R)-1-[2-[2-(3- hydroxycyclobutyl)indazol-5- yl]-3,6-dimethyl-4-oxo- chromen-8-yl]ethoxy]pyridine- 2-carboxamide | 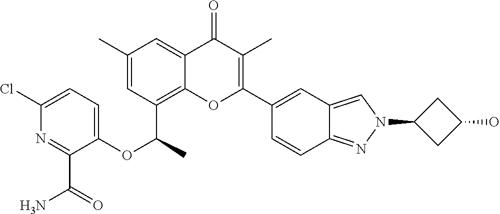 | 559 [M + H]⁺ |
| 135C | 6-Chloro-3-[(1R)-1-[2-[2-(2- hydroxyethyl)indazol-5-yl]-3,6- dimethyl-4-oxo-chromen-8- yl]ethoxy]-N-methoxy- pyridine-2-carboxamide | 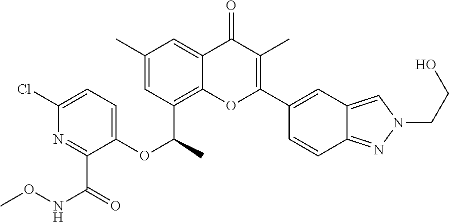 [SMILES: CONC(=O)c1nc(Cl)ccc1O[C@H](C)c1cc(C)cc2c(=O)c(C)c(-c3ccc4nn(CCO)cc4c3)oc12] | 561 [M − H]⁻ |

The following compounds in Table 32 can be made according to the foregoing Examples and/or using common general knowledge in synthetic chemistry.

TABLE 32

| | | | MS ES+ |
|---|---|---|---|
| Example # | Chemical Name | Structure | m/z |
| 146A | 8-[(1R)-1-[(6-Chloro-2- methylsulfonyl-3- pyridyl)oxy]ethyl]-3,6- dimethyl-2-(1- methylpyrazol-4- yl)chromen-4-one | 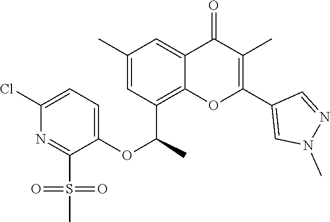 [SMILES: Cc1cc([C@@H](C)Oc2ccc(Cl)nc2S(C)(=O)=O)c2oc(-c3cnn(C)c3)c(C)c(=O)c2c1] | 488 [M + H]⁺ |
| 147A | 8-[(1R)-1-[[6-Chloro-2- (methylsulfonimidoyl)-3- pyridyl]oxy]ethyl]-3,6- dimethyl-2-(1- methylpyrazol-4- yl)chromen-4-one | 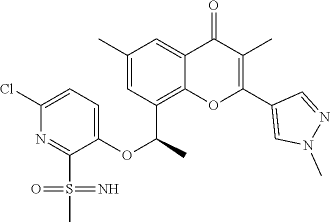 [SMILES: Cc1cc([C@@H](C)Oc2ccc(Cl)nc2S(C)(=N)=O)c2oc(-c3cnn(C)c3)c(C)c(=O)c2c1] | 487 [M + H]⁺ |
| 149A | N-[6-Chloro-3-[(1R)-1-[3,6- dimethyl-2-(1- methylpyrazol-4-yl)-4-oxo- chromen-8-yl]ethoxy]-2- pyridyl]methanesulfonamide | 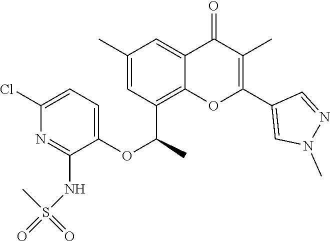 [SMILES: Cc1cc([C@@H](C)Oc2ccc(Cl)nc2NS(C)(=O)=O)c2oc(-c3cnn(C)c3)c(C)c(=O)c2c1] | 503 [M + H]⁺ |
| 26B | 3-[(1R)-1-[3,6-Dimethyl-2- (1-methylpyrazol-4-yl)-4- oxo-chromen-8-yl]ethoxy]- 6-fluoro-pyridine-2- sulfonamide | 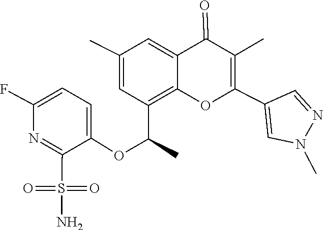 [SMILES: Cc1cc([C@@H](C)Oc2ccc(F)nc2S(N)(=O)=O)c2oc(-c3cnn(C)c3)c(C)c(=O)c2c1] | 473 [M + H]⁺ |
| 27B | N-[6-Chloro-3-[(1R)-1-[3,6- dimethyl-2-(1- methylpyrazol-4-yl)-4-oxo- chromen-8-yl]ethoxy]-2- pyridyl]-2-hydroxy- acetamide | 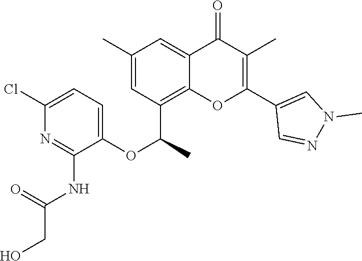 [SMILES: Cc1cc([C@@H](C)Oc2ccc(Cl)nc2NC(=O)CO)c2oc(-c3cnn(C)c3)c(C)c(=O)c2c1] | 483 [M + H]⁺ |
| 28B | 8-[(1R)-1-[[6-Chloro-2- (methylsulfonimidoyl)-3- pyridyl]oxylethyl]-2-[1-(2- hydroxyethyl)pyrazol-4-yl]- 3,6-dimethyl-chromen-4-one | 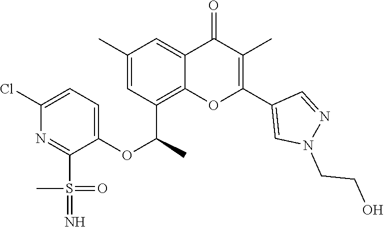 [SMILES: Cc1cc([C@@H](C)Oc2ccc(Cl)nc2S(C)(=N)=O)c2oc(-c3cnn(CCO)c3)c(C)c(=O)c2c1] | 517 [M + H]⁺ |
| 29B | 8-[(1R)-1-[(6-Chloro-2- methylsulfonyl-3- pyridyl)oxy]ethyl]-2-[1-(2- hydroxyethyl)pyrazol-4-yl]- 3,6-dimethyl-chromen-4-one | 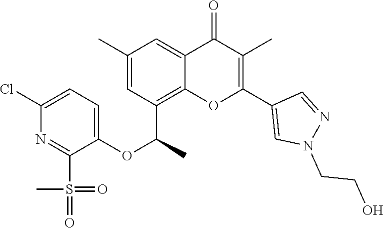 [SMILES: Cc1cc([C@@H](C)Oc2ccc(Cl)nc2S(C)(=O)=O)c2oc(-c3cnn(CCO)c3)c(C)c(=O)c2c1] | 518 [M + H]⁺ |
| 30B | 8-[(1R)-1-[(2-Amino-6- chloro-3-pyridyl)oxy]ethyl]- 3,6-dimethyl-2-(1- methylpyrazol-4- yl)chromen-4-one | 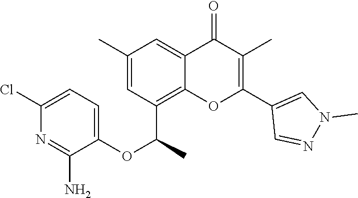 [SMILES: Cc1cc([C@@H](C)Oc2ccc(Cl)nc2N)c2oc(-c3cnn(C)c3)c(C)c(=O)c2c1] | 425 [M + H]⁺ |
| 31B | N-[6-Chloro-3-[(1R)-1-[3,6- dimethyl-2-(1- methylpyrazol-4-yl)-4-oxo- chromen-8-yl]ethoxy]-2- pyridyl]-N-methylsulfonyl- methanesulfonamide | 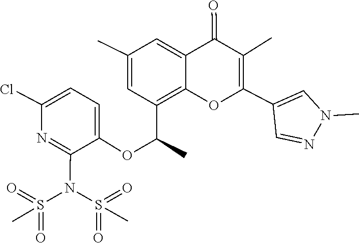 [SMILES: Cc1cc([C@@H](C)Oc2ccc(Cl)nc2N(S(C)(=O)=O)S(C)(=O)=O)c2oc(-c3cnn(C)c3)c(C)c(=O)c2c1] | 581 [M + H]⁺ |
| 136C | 6-Fluoro-3-[(1R)-1-[2-[1-(2- hydroxy-2-methyl- propyl)pyrazol-4-yl]-3,6- dimethyl-4-oxo-chromen-8- yl]ethoxy]-N-methoxy- pyridine-2-sulfonamide | 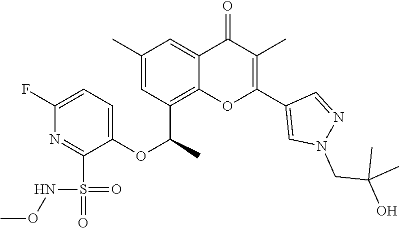 [SMILES: CONS(=O)(=O)c1nc(F)ccc1O[C@H](C)c1cc(C)cc2c(=O)c(C)c(-c3cnn(CC(C)(C)O)c3)oc12] | 561 [M + H]⁺ |

The following compounds in Table 33 can be made according to the foregoing Examples and/or using common general knowledge in synthetic chemistry.

TABLE 33

| Example | | |
|---|---|---|
| # | Chemical Name | Structure |
| 72A | 6-Chloro-3-[(1R)-1-[3,6- dimethyl-2-(1-methyltriazol-4- yl)-4-oxo-chromen-8- yl]ethoxy]pyridine-2- sulfonamide | 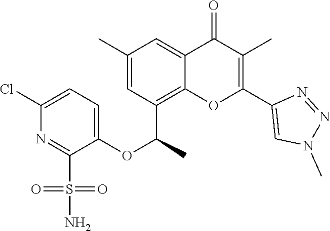 [SMILES: Cc1cc([C@@H](C)Oc2ccc(Cl)nc2S(N)(=O)=O)c2oc(-c3cn(C)nn3)c(C)c(=O)c2c1] |
| 73A | 6-Chloro-3-[(1R)-1-[2-(1- cyclopropylpyrazol-4-yl)-3,6- dimethyl-4-oxo-chromen-8- yl]ethoxy]pyridine-2- sulfonamide | 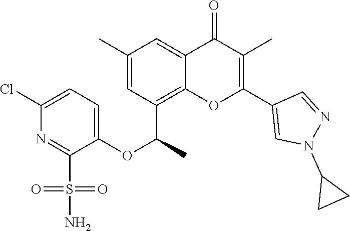 [SMILES: Cc1cc([C@@H](C)Oc2ccc(Cl)nc2S(N)(=O)=O)c2oc(-c3cnn(C4CC4)c3)c(C)c(=O)c2c1] |
| 75A | 6-Chloro-3-[(1R)-1-(3,6- dimethyl-2-oxazolo[5,4- b]pyridin-6-yl-4-oxo-chromen- 8-yl)ethoxy]pyridine-2- sulfonamide | 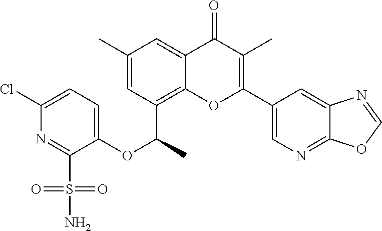 [SMILES: Cc1cc([C@@H](C)Oc2ccc(Cl)nc2S(N)(=O)=O)c2oc(-c3cnc4ocnc4c3)c(C)c(=O)c2c1] |
| 76A | 6-Chloro-3-[(1R)-1-[3,6- dimethyl-2-(2- methyloxazolo[5,4-b]pyridin-6- yl)-4-oxo-chromen-8- yl]ethoxy]pyridine-2- sulfonamide | 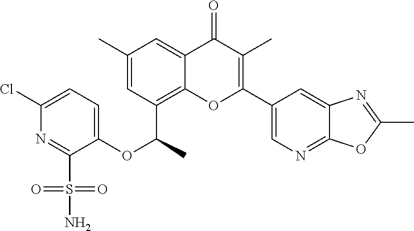 [SMILES: Cc1cc([C@@H](C)Oc2ccc(Cl)nc2S(N)(=O)=O)c2oc(-c3cnc4oc(C)nc4c3)c(C)c(=O)c2c1] |
| 77A | 6-Chloro-3-[(1R)-1-[2-(1H- indazol-5-yl)-3,6-dimethyl-4- oxo-chromen-8- yl]ethoxy]pyridine-2- sulfonamide | 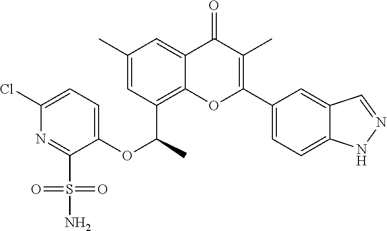 [SMILES: Cc1cc([C@@H](C)Oc2ccc(Cl)nc2S(N)(=O)=O)c2oc(-c3ccc4[nH]ncc4c3)c(C)c(=O)c2c1] |
| 79A | 6-Chloro-3-[(1R)-1-[2-(4,4- difluorocyclohexyl)-3,6- dimethyl-4-oxo-chromen-8- yl]ethoxy]pyridine-2- sulfonamide | 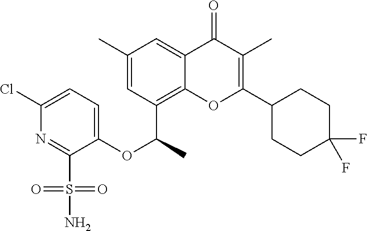 [SMILES: Cc1cc([C@@H](C)Oc2ccc(Cl)nc2S(N)(=O)=O)c2oc(C3CCC(F)(F)CC3)c(C)c(=O)c2c1] |
| 80A | 6-Chloro-3-[(1R)-1-(3,6- dimethyl-2-oxazolo[4,5- b]pyridin-6-yl-4-oxo-chromen- 8-yl)ethoxy]pyridine-2- sulfonamide | 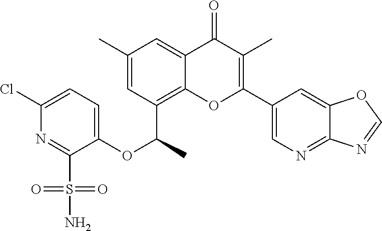 [SMILES: Cc1cc([C@@H](C)Oc2ccc(Cl)nc2S(N)(=O)=O)c2oc(-c3cnc4ncoc4c3)c(C)c(=O)c2c1] |
| 81A | 6-Chloro-3-[(1R)-1-[3,6- dimethyl-2-(2- methyloxazolo[4,5-b]pyridin-6- yl)-4-oxo-chromen-8- yl]ethoxy]pyridine-2- sulfonamide | 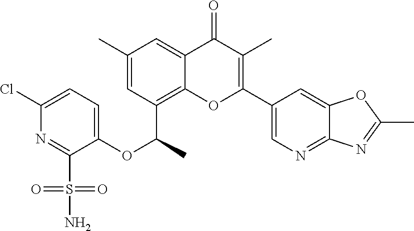 [SMILES: Cc1cc([C@@H](C)Oc2ccc(Cl)nc2S(N)(=O)=O)c2oc(-c3cnc4nc(C)oc4c3)c(C)c(=O)c2c1] |
| 82A | 6-Chloro-3-[(1R)-1-[2-[1- (difluoromethyl)pyrazol-4-yl]- 3,6-dimethyl-4-oxo-chromen-8- yl]ethoxy]pyridine-2- sulfonamide | 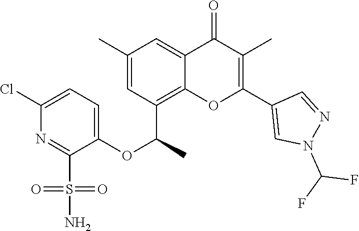 [SMILES: Cc1cc([C@@H](C)Oc2ccc(Cl)nc2S(N)(=O)=O)c2oc(-c3cnn(C(F)F)c3)c(C)c(=O)c2c1] |
| 84A | 6-Chloro-3-[(1R)-1-[3,6- dimethyl-2-(6-morpholino-3- pyridyl)-4-oxo-chromen-8- yl]ethoxy]pyridine-2- sulfonamide | 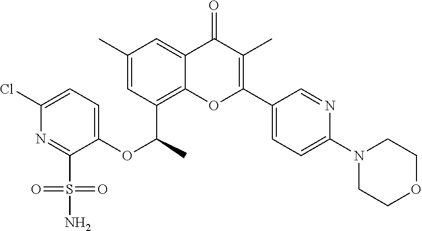 [SMILES: Cc1cc([C@@H](C)Oc2ccc(Cl)nc2S(N)(=O)=O)c2oc(-c3ccc(N4CCOCC4)nc3)c(C)c(=O)c2c1] |
| 85A | 6-Chloro-3-[(1R)-1-(3,6- dimethyl-2-oxazolo[4,5- b]pyridin-5-yl-4-oxo-chromen- [8-yl)ethoxy]pyridine-2- sulfonamide | 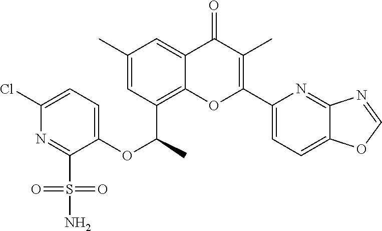 [SMILES: Cc1cc([C@@H](C)Oc2ccc(Cl)nc2S(N)(=O)=O)c2oc(-c3ccc4ocnc4n3)c(C)c(=O)c2c1] |
| 86A | 6-Chloro-3-[(1R)-1-[3,6- dimethyl-2-(2- methyloxazolo[4,5-b]pyridin-5- yl)-4-oxo-chromen-8- yl]ethoxy]pyridine-2- sulfonamide | 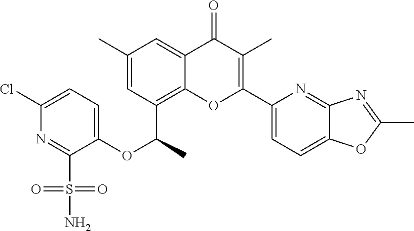 [SMILES: Cc1cc([C@@H](C)Oc2ccc(Cl)nc2S(N)(=O)=O)c2oc(-c3ccc4oc(C)nc4n3)c(C)c(=O)c2c1] |
| 87A | 6-Chloro-3-[(1R)-1-[2-(1- isopropylpyrazol-4-yl)-3,6- dimethyl-4-oxo-chromen-8- yl]ethoxy]pyridine-2- sulfonamide | 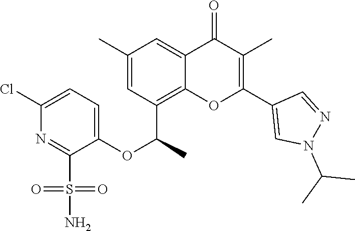 [SMILES: Cc1cc([C@@H](C)Oc2ccc(Cl)nc2S(N)(=O)=O)c2oc(-c3cnn(C(C)C)c3)c(C)c(=O)c2c1] |
| 88A | 6-Chloro-3-[(1R)-1-[2-[5-(2- hydroxyethyl)-3-pyridyl]-3,6- dimethyl-4-oxo-chromen-8- yl]ethoxy]pyridine-2- sulfonamide | 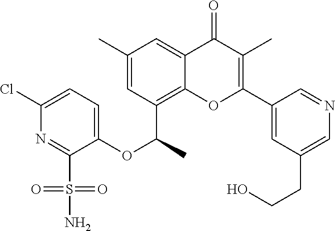 [SMILES: Cc1cc([C@@H](C)Oc2ccc(Cl)nc2S(N)(=O)=O)c2oc(-c3cncc(CCO)c3)c(C)c(=O)c2c1] |
| 90A | 6-Chloro-3-[(1R)-1-(3,6- dimethyl-4-oxo-2-thiazolo[5,4- b]pyridin-5-yl-chromen-8- yl)ethoxy]pyridine-2- sulfonamide | 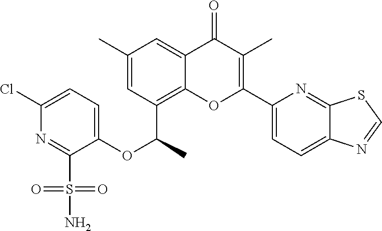 [SMILES: Cc1cc([C@@H](C)Oc2ccc(Cl)nc2S(N)(=O)=O)c2oc(-c3ccc4ncsc4n3)c(C)c(=O)c2c1] |
| 91A | 6-Chloro-3-[(1R)-1-[3,6- dimethyl-2-(2- methylthiazolo[5,4-b]pyridin-5- yl)-4-oxo-chromen-8- yl]ethoxy]pyridine-2- sulfonamide | 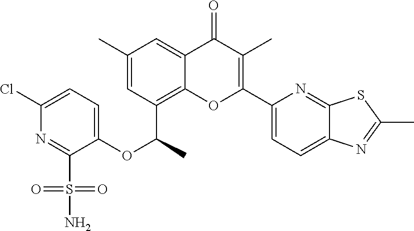 [SMILES: Cc1cc([C@@H](C)Oc2ccc(Cl)nc2S(N)(=O)=O)c2oc(-c3ccc4nc(C)sc4n3)c(C)c(=O)c2c1] |
| 92A | 3-[(1R)-1-[2-(2,1,3- Benzothiadiazol-5-yl)-3,6- dimethyl-4-oxo-chromen-8- yl]ethoxy]-6-chloro-pyridine-2- sulfonamide | 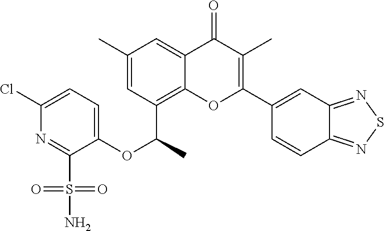 [SMILES: Cc1cc([C@@H](C)Oc2ccc(Cl)nc2S(N)(=O)=O)c2oc(-c3ccc4nsnc4c3)c(C)c(=O)c2c1] |
| 93A | 6-Chloro-3-[(1R)-1-(2- imidazo[1,2-a]pyrimidin-6-yl- 3,6-dimethyl-4-oxo-chromen-8- yl)ethoxy]pyridine-2- sulfonamide | 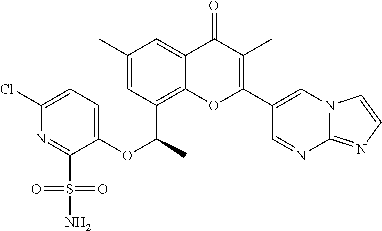 [SMILES: Cc1cc([C@@H](C)Oc2ccc(Cl)nc2S(N)(=O)=O)c2oc(-c3cnc4nccn4c3)c(C)c(=O)c2c1] |
| 94A | 6-Chloro-3-[(1R)-1-(3,6- dimethyl-4-oxo-2-pyrazolo[1,5- a]pyrimidin-6-yl-chromen-8- yl)ethoxy]pyridine-2- sulfonamide | 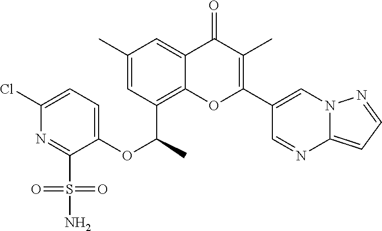 [SMILES: Cc1cc([C@@H](C)Oc2ccc(Cl)nc2S(N)(=O)=O)c2oc(-c3cnc4ccnn4c3)c(C)c(=O)c2c1] |
| 95A | 6-Chloro-N-cyclopropyl-3- [(1R)-1-[3,6-dimethyl-2-(1- methylpyrazol-4-yl)-4-oxo- chromen-8-yl]ethoxy]pyridine- 2-sulfonamide | 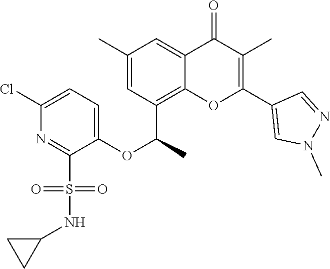 [SMILES: Cc1cc([C@@H](C)Oc2ccc(Cl)nc2S(=O)(=O)NC2CC2)c2oc(-c3cnn(C)c3)c(C)c(=O)c2c1] |
| 96A | 6-Chloro-N-cyclopropyl-3- [(1R)-1-[2-[1-(2- hydroxyethyl)pyrazol-4-yl]-3,6- dimethyl-4-oxo-chromen-8- yl]ethoxy]pyridine-2- sulfonamide | 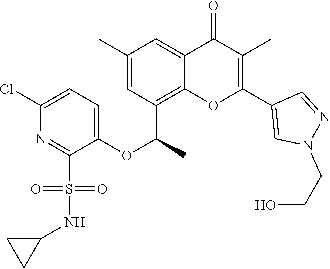 [SMILES: Cc1cc([C@@H](C)Oc2ccc(Cl)nc2S(=O)(=O)NC2CC2)c2oc(-c3cnn(CCO)c3)c(C)c(=O)c2c1] |
| 97A | 6-Chloro-N-cyclopropyl-3- [(1R)-1-[3,6-dimethyl-4-oxo-2- (3-pyridyl)chromen-8- yl]ethoxy]pyridine-2- sulfonamide | 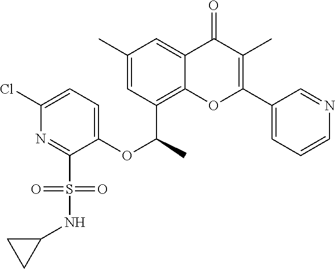 [SMILES: Cc1cc([C@@H](C)Oc2ccc(Cl)nc2S(=O)(=O)NC2CC2)c2oc(-c3cccnc3)c(C)c(=O)c2c1] |
| 98A | 6-Chloro-N-cyclopropyl-3- [(1R)-1-(3,6-dimethyl-4-oxo-2- pyrimidin-5-yl-chromen-8- yl)ethoxy]pyridine-2- sulfonamide | 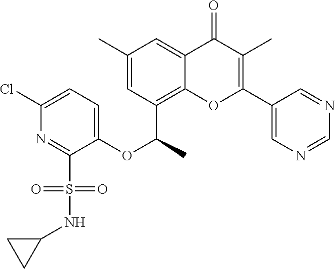 [SMILES: Cc1cc([C@@H](C)Oc2ccc(Cl)nc2S(=O)(=O)NC2CC2)c2oc(-c3cncnc3)c(C)c(=O)c2c1] |
| 99A | 3-[6-Chloro-3-[(1R)-1-[2-(2,2- dimethyl-[1,3]dioxolo[4,5- b]pyridin-6-yl)-3,6-dimethyl-4- oxo-chromen-8-yl]ethoxy]-2- pyridyl]-4H-1,2,4-oxadiazol-5- one | 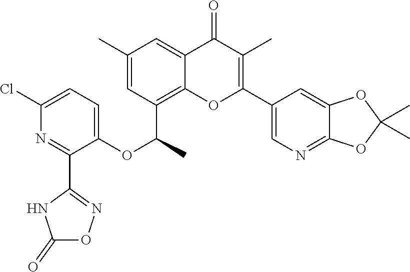 [SMILES: Cc1cc([C@@H](C)Oc2ccc(Cl)nc2-c2noc(=O)[nH]2)c2oc(-c3cnc4c(c3)OC(C)(C)O4)c(C)c(=O)c2c1] |
| 100A | 3-[6-Chloro-3-[(1R)-1-[2-(6,7- dihydro-5H- cyclopenta[b]pyridin-3-yl)-3,6- dimethyl-4-oxo-chromen-8- yl]ethoxy]-2-pyridyl]-4H-1,2,4- oxadiazol-5-one | 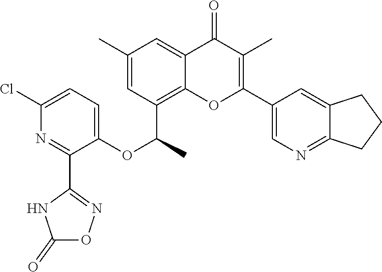 [SMILES: Cc1cc([C@@H](C)Oc2ccc(Cl)nc2-c2noc(=O)[nH]2)c2oc(-c3cnc4c(c3)CCC4)c(C)c(=O)c2c1] |
| 101A | 3-[6-Chloro-3-[(1R)-1-[2-(2- cyclopropylindazol-5-yl)-3,6- dimethyl-4-oxo-chromen-8- yl]ethoxy]-2-pyridyl]-4H-1,2,4- oxadiazol-5-one | 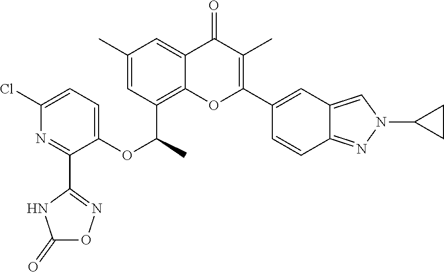 [SMILES: Cc1cc([C@@H](C)Oc2ccc(Cl)nc2-c2noc(=O)[nH]2)c2oc(-c3ccc4nn(C5CC5)cc4c3)c(C)c(=O)c2c1] |
| 117A | 6-Chloro-3-[(1R)-1-[3,6- dimethyl-2-(2-methyl-7aH- imidazo[4,5-b]pyridin-6-yl)-4- oxo-chromen-8- yl]ethoxy]pyridine-2- carboxamide | 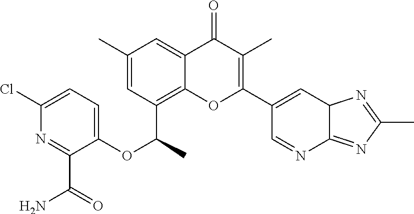 [SMILES: CC1=NC2C=C(c3oc4c([C@@H](C)Oc5ccc(Cl)nc5C(N)=O)cc(C)cc4c(=O)c3C)C=NC2=N1] |
| 118A | 6-Chloro-3-[(1R)-1-(2-furo[3,2- c]pyridin-3-yl-3,6-dimethyl-4- oxo-chromen-8- yl)ethoxy]pyridine-2- carboxamide | 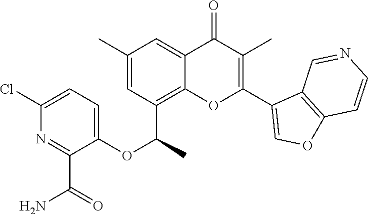 [SMILES: Cc1cc([C@@H](C)Oc2ccc(Cl)nc2C(N)=O)c2oc(-c3coc4ccncc34)c(C)c(=O)c2c1] |
| 122A | 6-Chloro-3-[(1R)-1-[2-(5,7- dihydro-4H-pyrazolo[1,5- c][1,3]oxazin-3-yl)-3,6- dimethyl-4-oxo-chromen-8- yl]ethoxy]pyridine-2- carboxamide | 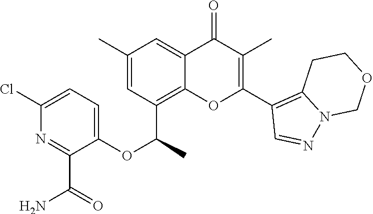 [SMILES: Cc1cc([C@@H](C)Oc2ccc(Cl)nc2C(N)=O)c2oc(-c3cnn4c3CCOC4)c(C)c(=O)c2c1] |
| 124A | 6-Chloro-3-[(1R)-1-[3,6- dimethyl-4-oxo-2-[2- (trifluoromethyl)indazol-5- yl]chromen-8- yl]ethoxy]pyridine-2- carboxamide | 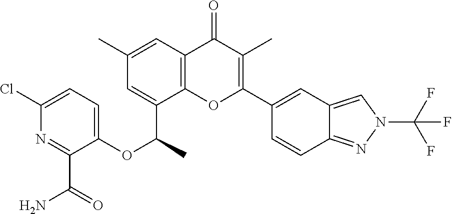 [SMILES: Cc1cc([C@@H](C)Oc2ccc(Cl)nc2C(N)=O)c2oc(-c3ccc4nn(C(F)(F)F)cc4c3)c(C)c(=O)c2c1] |
| 125A | 6-Chloro-3-[(1R)-1-(3,6- dimethyl-2-oxazolo[4,5- b]pyridin-5-yl-4-oxo-chromen- 8-yl)ethoxy]pyridine-2- carboxamide | 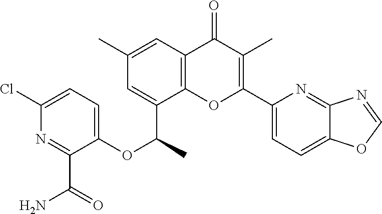 [SMILES: Cc1cc([C@@H](C)Oc2ccc(Cl)nc2C(N)=O)c2oc(-c3ccc4ocnc4n3)c(C)c(=O)c2c1] |
| 127A | 6-Chloro-3-[(1R)-1-(2- imidazo[1,2-a]pyrimidin-6-yl- 3,6-dimethyl-4-oxo-chromen-8- yl)ethoxy]pyridine-2- carboxamide | 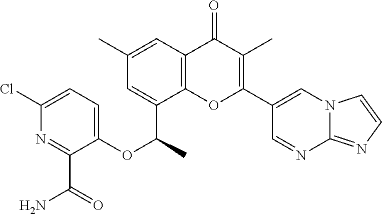 [SMILES: Cc1cc([C@@H](C)Oc2ccc(Cl)nc2C(N)=O)c2oc(-c3cnc4nccn4c3)c(C)c(=O)c2c1] |
| 129A | 6-Chloro-3-[(1R)-1-[3,6- dimethyl-4-oxo-2-[3- (trifluoromethyl)cyclopenta- 1,4-dien-1-yl]chromen-8- yl]ethoxy]pyridine-2- carboxamide | 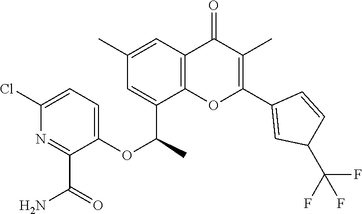 [SMILES: Cc1cc([C@@H](C)Oc2ccc(Cl)nc2C(N)=O)c2oc(C3=CC(C(F)(F)F)C=C3)c(C)c(=O)c2c1] |
| 131A | 6-Chloro-3-[(1R)-1-(3,6- dimethyl-4-oxo-2-thiazolo[5,4- b]pyridin-5-yl-chromen-8- yl)ethoxy]pyridine-2- carboxamide | 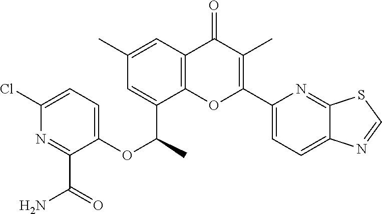 [SMILES: Cc1cc([C@@H](C)Oc2ccc(Cl)nc2C(N)=O)c2oc(-c3ccc4ncsc4n3)c(C)c(=O)c2c1] |
| 133A | 6-Chloro-3-[(1R)-1-[3,6- dimethyl-2-(1- methylpyrazolo[5,4-b]pyridin- 6-yl)-4-oxo-chromen-8- yl]ethoxy]pyridine-2- carboxamide | 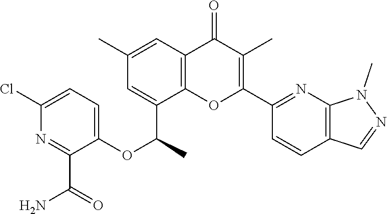 [SMILES: Cc1cc([C@@H](C)Oc2ccc(Cl)nc2C(N)=O)c2oc(-c3ccc4cnn(C)c4n3)c(C)c(=O)c2c1] |
| 134A | 6-Chloro-3-[(1R)-1-(3,6- dimethyl-2-oxazolo[5,4- b]pyridin-6-yl-4-oxo-chromen- 8-yl)ethoxy]pyridine-2- carboxamide | 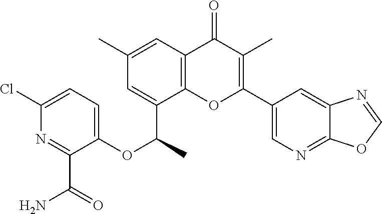 [SMILES: Cc1cc([C@@H](C)Oc2ccc(Cl)nc2C(N)=O)c2oc(-c3cnc4ocnc4c3)c(C)c(=O)c2c1] |
| 135A | 6-Chloro-3-[(1R)-1-[3,6- dimethyl-2-(2- methyloxazolo[4,5-b]pyridin-6- yl)-4-oxo-chromen-8- yl]ethoxy]pyridine-2- carboxamide | 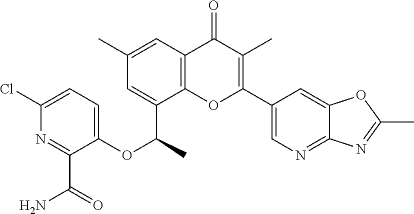 [SMILES: Cc1cc([C@@H](C)Oc2ccc(Cl)nc2C(N)=O)c2oc(-c3cnc4nc(C)oc4c3)c(C)c(=O)c2c1] |
| 136A | 6-Chloro-3-[(1R)-1-[3,6- dimethyl-2-(2- methylthiazolo[5,4-b]pyridin-5- yl)-4-oxo-chromen-8- yl]ethoxy]pyridine-2- carboxamide | 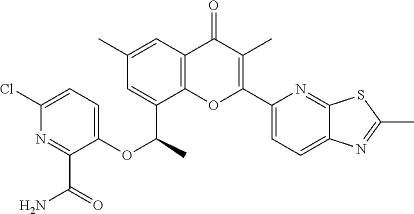 [SMILES: Cc1cc([C@@H](C)Oc2ccc(Cl)nc2C(N)=O)c2oc(-c3ccc4nc(C)sc4n3)c(C)c(=O)c2c1] |
| 138A | 6-Chloro-3-[(1R)-1-(3,6- dimethyl-2-oxazolo[4,5- b]pyridin-6-yl-4-oxo-chromen- 8-yl)ethoxy]pyridine-2- carboxamide | 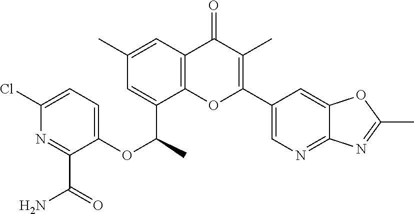 [SMILES: Cc1cc([C@@H](C)Oc2ccc(Cl)nc2C(N)=O)c2oc(-c3cnc4nc(C)oc4c3)c(C)c(=O)c2c1] |
| 139A | 6-Chloro-3-[(1R)-1-[3,6- dimethyl-2-(2- methyloxazolo[4,5-b]pyridin-5- yl)-4-oxo-chromen-8- yl]ethoxy]pyridine-2- carboxamide | 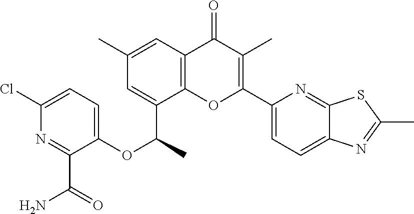 [SMILES: Cc1cc([C@@H](C)Oc2ccc(Cl)nc2C(N)=O)c2oc(-c3ccc4nc(C)sc4n3)c(C)c(=O)c2c1] |
| 142A | 3-[(1R)-1-[3,6-Dimethyl-2-(2- methyl-7aH-imidazo[4,5- b]pyridin-6-yl)-4-oxo-chromen- 8-yl]ethoxy]-6-fluoro-pyridine- 2-carboxamide | 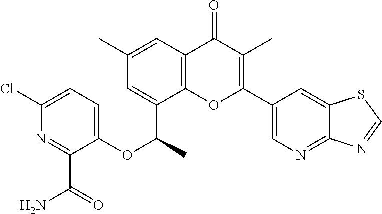 [SMILES: Cc1cc([C@@H](C)Oc2ccc(Cl)nc2C(N)=O)c2oc(-c3cnc4ncsc4c3)c(C)c(=O)c2c1] |
| 143A | 6-Chloro-3-[(1R)-1-[3-cyano-6- methyl-2-(2-methyl-7aH- imidazo[4,5-b]pyridin-6-yl)-4- oxo-chromen-8- yl]ethoxy]pyridine-2- carboxamide | 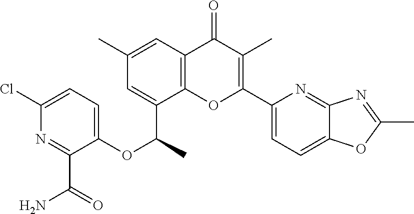 [SMILES: Cc1cc([C@@H](C)Oc2ccc(Cl)nc2C(N)=O)c2oc(-c3ccc4oc(C)nc4n3)c(C)c(=O)c2c1] |
| 144A | N-[[6-Chloro-3-[(1R)-1-(3,6- dimethyl-4-oxo-2-phenyl- chromen-8-yl)ethoxy]-2- pyridyl]sulfonyl]acetamide | 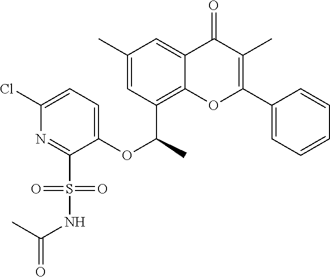 [SMILES: CC(=O)NS(=O)(=O)c1nc(Cl)ccc1O[C@H](C)c1cc(C)cc2c(=O)c(C)c(-c3ccccc3)oc12] |
| 145A | N-[[6-Chloro-3-[(1R)-1-[3,6- dimethyl-2-(1-methylpyrazol-4- yl)-4-oxo-chromen-8- yl]ethoxy]-2- pyridyl]sulfonyl]acetamide | 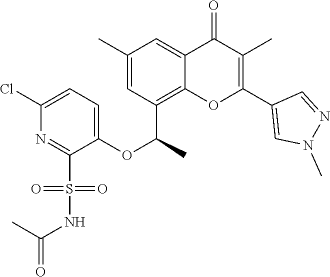 [SMILES: CC(=O)NS(=O)(=O)c1nc(Cl)ccc1O[C@H](C)c1cc(C)cc2c(=O)c(C)c(-c3cnn(C)c3)oc12] |
| 150A | N-[6-Chloro-3-[(1R)-1-[3,6- dimethyl-2-(2-methylindazol-5- yl)-4-oxo-chromen-8- yl]ethoxy]-2- pyridyl]methanesulfonamide | 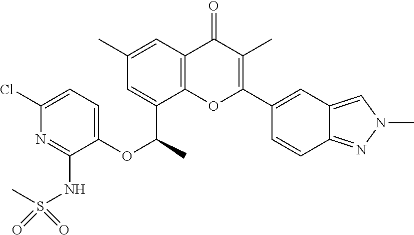 [SMILES: Cc1cc([C@@H](C)Oc2ccc(Cl)nc2NS(C)(=O)=O)c2oc(-c3ccc4nn(C)cc4c3)c(C)c(=O)c2c1] |
| 151A | N-[6-Chloro-3-[(1R)-1-(3,6- dimethyl-4-oxo-2-phenyl- chromen-8-yl)ethoxy]-2- pyridyl]methanesulfonamide | 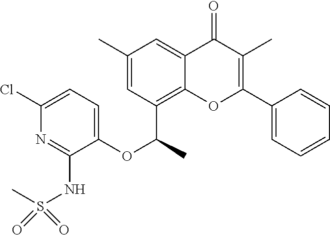 [SMILES: Cc1cc([C@@H](C)Oc2ccc(Cl)nc2NS(C)(=O)=O)c2oc(-c3ccccc3)c(C)c(=O)c2c1] |

PI3K-Alpha Kinase (PI3Kα) Activity: Wild-Type PI3Kα, H1047R Mutant PI3Kα, and E545K Mutant PI3Kα In Vitro Cell Based Assays and Determination of IC50 Values for Inhibitors

The MDA-MB-453 (ATCC-HTB-131) cell line (PI3Kα H1047R) and MCF-7 (ATCC-HTB-22) cell line (PI3Kα E545K), MDA-MB-361 (ATCC-HTB-27) cell line (PI3Kα E545K), and SKBR3 (ATCC-HTB-30) cell line (wild type PI3Kα) were obtained from the American Type Culture Collection (Manassas, VA). MDA-MB-453 cells were maintained in Dulbecco's Modified Eagle Media (DMEM, Gibco 12430) supplemented with 10% Fetal Bovine Serum, heat inactivated (FBS HI, Gibco 10082), 1× non-essential amino acids (NEAA, Gibco 11140), 1 mM sodium pyruvate (Gibco 11360) and 1× Anti-Anti (Gibco 15240). MCF-7 cells were maintained in Minimum Essential Media (MEM) (Gibco 11095) supplemented with 10% Fetal Bovine Serum, heat inactivated (FBS HI, Gibco 10082), 1× non-essential amino acids (NEAA, Gibco 11140), 1 mM sodium pyruvate (Gibco 11360), 1× Anti-Anti (Gibco 15240) and 10 μg/mL human insulin (Sigma I 9278). MDA-MB-361 cells were maintained in Dulbecco's Modified Eagle Media (DMEM, Gibco 12430) supplemented with 20% Fetal Bovine Serum, heat inactivated (FBS HI, Gibco 10082), 1× non-essential amino acids (NEAA, Gibco 11140), 1 mM sodium pyruvate (Gibco 11360) and 1× Anti-Anti (Gibco 15240). SKBR3 cells were maintained in McCoy's 5A (Gibco 16600) supplemented with 10% Fetal Bovine Serum, heat inactivated (FBS HI, Gibco 10082), and 1× Anti-Anti (Gibco 15240). Cultures were maintained in a humidified incubator at 37° C. under 5% CO₂/95% air.

For compound testing in 0% FBS, MDA-MB-453, MCF-7 and SKBR3 cells were seeded at a density of 1.5×10⁴, 1.0×10⁴, and 1.0×10⁴ cells, respectively, per well in white 384-well plates in 20 μl of Minimum Essential Media (MEM) assay media with 1×NEAA, 1 mM sodium pyruvate, and 1 μg/mL human insulin (Sigma 19278) (Assay Medium); while MDA-MB-361 cells were seeded at a density of 1.5×10⁴ per well in white 384-well plates in 20 μl Assay Medium without insulin. After plating, cells were allowed to attach overnight. Compounds dissolved in 10 mM stock solutions in DMSO were serially diluted 1:3 in DMSO to generate a 10-point dilution series and plated using an acoustic liquid handler system (Echo 550 Series Liquid Handler, Labcyte). A 5× intermediate compound dilution plate in MEM with 1×NEAA and 1 mM sodium pyruvate (150 μM starting compound concentration in 1.5% DMSO) was then prepared. Five μl of the intermediate serially diluted compounds were added to the cell plate to final concentrations ranging from 30 mM to 0.0015 mM in 0.3% DMSO. 0.3% DMSO alone was used to establish the maximum (MAX) signal and GDC-0032 at a final concentration of 1 μM was used as a reference compound for the minimum (MIN) signal. After 3 hours treatment, the medium was removed, and the cells were lysed in 10 μL of 1× SureFire Lysis buffer with shaking for 10 minutes at room temperature. The Acceptor Mix (Reaction Buffer 1+Reaction Buffer 2+Activation Buffer+SureFire Ultra Acceptor Beads) was prepared by diluting Activation buffer 25-fold in combined Reaction Buffer 1 and Reaction Buffer 2.

The Acceptor beads were diluted 50-fold in the combined Reaction Buffers. Five μL of Acceptor Mix was added to each well, the plate was sealed and covered with foil and incubated for 1 hour at room temperature. The Donor Mix (dilution buffer+SureFire Ultra Donor Beads) was prepared by diluting Donor Beads 50-fold in dilution buffer. Five μL of the Donor Mix was added to each well and the plate sealed and covered with foil and incubated for 1-2 hours at room temperature in the dark. The plates were read on a Neo2 plate reader instrument from Biotek using standard AlphaLisa settings.

Compounds were tested in duplicate and the % inhibition at each compound concentration was used to generate two dose response curves. One IC50 was generated using average % inhibition at each compound concentration. The data were processed using the Genedata-Screener tool. Relative IC₅₀ values were determined using luminescence units by calculating percent inhibition with respect to the in-plate “MIN” (GDC-0032 reference control) and “MAX” (DMSO) controls. The data was analyzed using a 4-parameter nonlinear logistic equation (four-parameter logistic concentration-response curve):

Y=bottom+[(top−bottom)/1+(X/IC₅₀)slope], where
Y=% inhibition,
X=concentration of inhibitor,
bottom=minimum value of y attained by curve-fit,
top=maximum value of y attained by curve-fit and
slope=steepness of curve at the IC₅₀.

% Inhibition=[(signal at X−median Min)/(median Max−median Min)]×100
IC₅₀: concentration of compound that reduces a given response (ligand binding, enzyme response) by 50%.
Relative IC₅₀: concentration giving half the compound's maximum response.

Compound selectivity was calculated by dividing IC₅₀ in SKBR3 by IC₅₀ in cell lines harboring mutant PI3Kα (MDA-MB-453: H1047R; MCF-7 and MDA-MB-361: E545K).

Potency of Tested Compounds in MDA-MB-453 (ATCC-HTB-131) Cell Line (PI3Kα H1047R).

Selected compounds of the Examples were tested in the above cell-based PI3Kα H1047R mutant assay. Examples 1, 2, 10, 9A, 11A-18A, 26A-30A, 32A, 35A, 37A, 39A, 42A, 46A, 48A, 49A, 52A, 57A, 61A, 64A-66A, 68A, 74A, 83A, 103A-105A, 108A, 123A, 146A, 2B-7B, 12B, 17B, 22B, 24B-27B, 29B, 30B, 6C, 8C-13C, 17C-19C, 22C-26C, 30C, 31C, 33C, 36C, 38C, 40C, 45C, 53C, 58C, 63C, 65C, 66C, 69C, 71C-77C, 81C, 85C, 88C, 90C, 94C, 95C, 98C-107C, 109C-111C, 114C-126C, 127C-130C, and 132C-136C exhibited IC₅₀ values of less than 100 nM for the PI3Kα H1047R mutant.

Selectivity of Tested Compounds in MDA-MB-453 (ATCC-HTB-131) Cell Line (PI3Kα H1047R) Versus Wild Type.

Selected compounds of the Examples were tested in the above cell-based MDA-MB-453 PI3Kα H1047R mutant and wild type assays. Examples 1, 2, 5, 8-10, 4A-6A, 8A, 11A, 13A-17A, 22A, 27A, 32A-36A, 38A, 39A, 42A, 46A, 47A, 49A, 52A, 55A, 56A, 61A, 64A, 74A, 78A, 83A, 102A, 103A, 108A, 115A, 116A, 137A, 146A, 147A, 1B-8B, 17B, 25B, 26B, 28B-30B, 3C, 6C, 9C, 12C, 13C, 15C, 17C, 21C, 22C, 24C, 25C, 27C, 29C-32C, 34C, 35C, 47C, 56C, 68C, 73C, 74C, 81C, 86C-89C, 91C, 92C-97C, 100C, 101C, 104C-107C, 109C, 110C, 111C, 113C-115C, 119C, 120C, and 122C-127C, exhibited greater than about 20× fold-selectivity for PI3K-Alpha kinase H1047R mutant versus wild-type.

Selected compounds of the Examples were tested in the above cell-based PI3Kα H1047R mutant and wild type assays. Examples 8, 10, 14A, 15A, 35A, 38A, 42A, 74A, 83A, 2B, 5B, 7B, 28B, 3C, 9C, 12C, 13C, 15C, 17C, 21C, 22C, 29C, 31C, 35C, 47C, 56C, 68C, 73C, 74C, 86C-89C, 92C-97C, 106C, 109C, 101C, 104C, 107C, 110C, 111C, 113C-115C, 120C, 122C, 125C-127C, exhibited greater than about 50× fold-selectivity for PI3K-Alpha kinase H1047R mutant versus wild-type.

Potency of Tested Compounds in MCF-7 (ATCC-HTB-22) Cell Line (PI3Kα E545K)

Selected compounds of the Examples were tested in the above cell-based MCF-7 PI3Kα E545K mutant assay. Examples 1, 2, 10, 9A-14A, 16A, 17A, 19A, 20A, 22A, 23A, 25A, 26A, 28A-33A, 35A-37A, 46A, 52A, 57A-63A, 65A, 66A, 68A, 69A, 71A, 74A, 83A, 104A-113A, 116A, 119A, 120A-123A, 126A, 128A, 140A, 141A, 146A, 149A, 2B-4B, 6B-12B, 14B, 15B, 17B-20B, 22B, 24B-27B, 29B, 30B, 7C-10C, 12C, 19C, 22C-25C, 31C, 36C-38C, 40C-43C, 45C, 46C, 48C, 52C-55C, 58C-63C, 64C-66C, 68C-70C, 72C-77C, 79C-85C, 94C, 98C, 99C-101C, 103C-105C, 107C, 109C, 110C, 114C, 115C, 117C, 119C, 120C, 122C-124C, 126C, 127C, 129C, 130C, 132C, 133C, and 136C exhibited IC₅₀ values of less than 500 nM for the MCF-7 PI3Kα E545K mutant.

Potency of Tested Compounds in MDA-MB-361 (ATCC-HTB-22) Cell Line (PI3Kα E545K)

Selected compounds of the Examples were tested in the above cell-based MDA-MB-361 PI3Kα E545K mutant assay. Examples 11A, 13A, 14A, 15A, 16A, 17A, 25A, 26A, 28A, 29A, 30A, 37A, 40A, 46A, 48A, 50A, 51A, 52A, 57A, 58A, 59A, 60A, 61A, 63A, 66A, 68A, 74A, 104A, 119A, 120A, 123A, 146A, 149A, 2B, 3B, 4B, 6B, 7B, 12B, 17B, 22B, 24B25B, 27B, 29B, 30B, 18C, 22C, 23C, 31C, 36C, 38C, 40C, 43C, 50C, 53C, 58C, 61C, 63C, 65C, 66C, 69C, 72C, 74C, 75C, 76C, 77C, 79C, 80C, 81C, 83C, 84C, 85C, 90C, 98C, 99C, 100C, 101C, 102C, 105C, 106C, 107C, 110C, 115C, 117C, 118C, 119C, 121C, 122C, 124C, 126C, 129C, 130C, 132C, 133C, and 134C exhibited IC₅₀ values of less than 500 nM for the MDA-MB-361 PI3Kα E545K mutant.

Metabolic Stability and Intrinsic Clearance in Liver Microsomes (MICS)

One objective of this assay is to measure in vitro metabolic stability of a test compound in liver microsomes of one or more different species. The concentration of a control compound in the reaction system is evaluated by LC/MS/MS for calculating intrinsic clearance of a test compound and estimating the stability of the control compound and test compound in liver microsomes of one or more different species.

Microsomes (MICS). Human Liver Microsomes (HLM), Monkey Liver Microsomes (MKLM), Dog Liver Microsomes (DLM), Rat Liver Microsomes (RLM), and Mouse Liver Microsomes (MLM) are obtained at a concentration of 20 mg/mL protein from commercial sources and stored in a −80° C. freezer. Prior to use, liver microsomes are removed from the freezer and allowed to thaw in a 37° C. water bath and then stored on wet ice.

Stock Solutions. Test Compound (TC) Stock Solutions are prepared in DMSO at a concentration of 10 mM and stored for later use. Positive Control (PC) Stock Solutions of verapamil·HCL (FW: 491.1 g/mol) in DMSO are prepared at a concentration of 10 mM (4.91 mg/mL), and stored at the condition same as the stock solutions of test compounds.

Phosphate Buffer. Phosphate Buffer (100 mM, pH 7.4) is prepared by combining a Solution A and a Solution B as follows.

To prepare Solution A, 7.098 g of disodium hydrogen phosphate was combined with 500 mL of pure water and sonicated to dissolve. To prepare the Solution B, 3.400 g of potassium dihydrogen phosphate was combined with 250 mL of pure water and sonicated to dissolve. Solution A was placed on a stirrer and Solution B was slowly added into Solution A until the pH reached 7.4.

NADPH Solution. A 10 mM NADPH Solution is prepared by dissolving NADPH (MW: 833.4 g/mol) at 8.334 mg/mL in phosphate buffer (100 mM, pH 7.4).

Microsome Master Solution. A Microsome Master Solution is prepared by adding 6.25 μL microsomes (20 mg/mL) to 216.25 μL Phosphate Buffer (100 mM). The final concentration is Phosphate Buffer (100 mM); microsomes (0.5 mg/mL).

Dilution of Stock Solutions. The TC Stock Solutions (10 mM in DMSO) and the PC Stock Solutions (10 mM in DMSO) are diluted by adding 3 μL of the TC Stock Solution or the PC Stock Solution to 297 μL of 50% acetonitrile/50% water.

Incubation. Two separated experiments were performed as follows. a) With Cofactors (NADPH): 25 μL of 10 mM NADPH was added to the incubations. The final concentrations of

microsomes and NADPH were 0.5 mg/mL and 1 mM, respectively. b) Without Cofactors (NADPH): 25 μL of 100 mM Phosphate buffer was added to the incubations. The final concentration of microsomes was 0.5 mg/mL. The mixture was pre-warmed at 37° C. for 10 minutes.

The reaction was started with the addition of 2.5 μL of 100 μM PC solution or TC solutions.

Verapamil was used as PC in this study. The final concentration of test compound or control compound was 1 μM. The incubation solution was incubated in water batch at 37° C.

Reaction Termination. Aliquots of 30 μL were taken from the reaction solution at 0.5, 5, 15, 30 and 60 minutes and added to plates. The reaction was stopped by the addition of 5 volumes of cold acetonitrile and a solution containing 100 nM alprazolam, 200 nM caffeine and 100 nM tolbutamide.

Sampling. The plates containing the terminated reactions were centrifuged at 4000 rpm for 30 minutes. Supernatant of each compound (40 μL) was transferred into a 96-well analysis plate.

Four compound samples are pooled together into one cassette and diluted by adding 160 μL of pure water. All incubations are performed in duplicate.

Analysis and Data Processing. Quantitative LC-MS analysis is performed, and peak areas are determined from extracted ion chromatograms. Percent parent remaining was calculated from peak area of TC or PC. The slope value, k, is determined by linear regression of the natural logarithm of percent parent remaining vs. incubation time curve.

The in vitro half-life (in vitro t_{1/2}) is determined from the slope value:

in vitro t_{1/2}=−(0.693/k)

Conversion of the in vitro t_{1/2} (in min) into the in vitro intrinsic clearance (in vitro CLᵢₙₜ, in μL/min/mg proteins) is done using the following equation:

$$in vitro {CL}_{int} = ( \frac{0.693}{( {t}_{1 / 2} )} ) * ( \frac{volume of incubation ( µL )}{amount of proteins ( mg )} )$$

Calculations of Scaled-up CLₕₑₚ (mL/min/kg), predicted CLᵢₙₜ(mL/min/kg) and EH may be performed using equations and scaling factors for Human, Monkey, Dog, Rat, and Mouse microsomes as known in the art.

Intrinsic Clearance of Test Compounds.

In the above assay, compounds of Examples indicated herein were tested in human MICS and exhibited metabolic stability as determined by calculating intrinsic clearance values. Examples 2-6, 1A-8A, 10A, 11A, 13A-16A, 18A, 20A, 23A, 24A, 26A, 27A, 31A, 33A-36A, 38A, 39A, 41A, 42A, 46A, 47A, 50A-56A, 58A-61A, 64A, 66A, 67A, 83A, 102A, 105A, 128A, 130A, 137A, 141A, 147A, 149A, 1B, 2B, 4B, 5B, 7B-9B, 16B, 17B, 19B, 23B-26B, 1C-6C, 8C, 10C, 14C-17C, 19C, 21C, 22C, 24C, 25C, 27C-35C, 37C, 39C, 42C, 44C, 46C, 47C, 52C, 58C, 60C-6C, 73C, 74C, 75C, 78C, 79C, 80C, 81C, 86C-98C, 99C, 103C, 109C, 101C, 105C, 108C, 110C, 111C, 115C, 119C, 122C-126C, 128C, and 135C exhibited an intrinsic clearance of less than about 50 μL/min/mg in human MICS.

Metabolic Stability and Intrinsic Clearance in Hepatocytes (HEPS)

One objective of this assay is to measure in vitro metabolic stability of a test compound in hepatocytes of one or more different species. The concentration of a control compound in the reaction system is evaluated by LC/MS/MS for calculating intrinsic clearance of a test compound and estimating the stability of the control compound and test compound in hepatocytes of one or more different species.

Hepatocytes (HEPS).

Hepatocytes were purchased from commercial sources and stored at less than −150° C.

Stock Solutions. Test Compound (TC) Stock Solutions are prepared in DMSO at a concentration of 10 mM and stored for later use. Positive Control (PC) Stock Solutions of verapamil·HCL (FW: 491.1 g/mol) in DMSO are prepared at a concentration of 10 mM (4.91 mg/mL), and stored at the condition same as the stock solutions of test compounds.

Preparation of Hepatocytes.

Incubation medium (William's E Medium supplemented with GlutaMAX) and hepatocyte thawing medium were warmed in a 37° C. water bath for at least 30 minutes prior to use.

Vials of cryopreserved hepatocytes were removed from storage, depressurized, and thawed in a 37° C. water bath. The contents were poured into a 50 mL thawing medium conical tube containing the hepatocyte thawing medium that had been pre-warmed to 37° C. Vials were centrifuged at 100 g for 5 minutes at room temperature. The thawing medium was aspirated and the hepatocytes were re-suspended with 3-4 mL serum-free incubation medium.

Cell viability and density were counted and the cell density was diluted with serum-free incubation medium to a working cell density of 0.5×10⁶ viable cells/mL

Dilution of Stock Solutions. The TC Stock Solutions (10 mM in DMSO) and the PC Stock Solutions (10 mM in DMSO) are diluted to 100 μM by adding 2 μL of the TC Stock Solution or the PC Stock Solution to 198 μL of 50% acetonitrile/50% water.

Incubation.

The hepatocyte suspension (247.5 μL) was pipetted into each well of a 96-well non-coated plate. The plate was placed in an incubator to allow the hepatocytes to warm for 10 minutes. TC (2.5 μL of the 100 μM TC solution) or PC (2.5 μL of the 100 μM PC solution) were pipetted into respective wells of the 96-well non-coated plate to start the reaction. The plate was returned to the incubator and samples were taken at designated time points.

Reaction Termination. Well contents were transferred in 25 μL aliquots at time points of 0.5, 30, 60, 90, 120 and 240 minutes to plates. The aliquots were then mixed with 12 volumes (300 L) of acetonitrile containing the internal standard (100 nM alprazolam, 200 nM caffeine and 100 nM tolbutamide) to terminate the reaction.

Sampling. The plates containing the terminated reactions were centrifuged at 4000 rpm for 40 minutes. Supernatant of each compound (100 μL) was transferred into a 96-well analysis plate.

Four compound samples are pooled together into one cassette and diluted by adding 160 μL of pure water. All incubations are performed in duplicate. Aliquots of 100 μL of the supernatant were diluted by 100 μL ultra-pure water, and the mixture was used for LC/MS/MS analysis. All incubations were performed in duplicate.

Analysis and Data Processing. Quantitative LC-MS analysis is performed, and peak areas are determined from extracted ion chromatograms. Percent parent remaining was calculated from peak area of TC or PC. The slope value, k, is determined by linear regression of the natural logarithm of percent parent remaining vs. incubation time curve.

The in vitro half-life (in vitro t_{1/2}) is determined from the slope value:

in vitro t_{1/2}=−(0.693/k)

Conversion of the in vitro t_{1/2} (in min) into the in vitro intrinsic clearance (in vitro CLᵢₙₜ, in μL/min/mg proteins) is done using the following equation:

$$in vitro {CL}_{i n t} = ( \frac{0.693}{( {t}_{1 / 2} )} ) * ( \frac{volume of incubation ( µL )}{amount of proteins ( mg )} )$$

Calculations of Scaled-up CLₕₑₚ (mL/min/kg), predicted CLᵢₙₜ(mL/min/kg) and EH may be performed using equations and scaling factors for Human, Monkey, Dog, Rat, and Mouse microsomes as known in the art.

Intrinsic Clearance of Test Compounds.

In the above assay, compounds of Examples indicated herein were tested in human HEPS and exhibited metabolic stability as determined by calculating intrinsic clearance values. Examples 1-7, 1A-11A, 13A, 14A, 16A-21A, 23A-28A, 31A, 33A-43A, 46A, 47A, 49A-64A, 66A-71A, 74A, 83A, 102A, 105A, 108A, 111A-114A, 116A, 119A-121A, 123A, 126A, 128A, 130A, 132A, 137A, 141A, 147A, 149A, 1B-10B, 12B-17B, 19B-31B, 1C-7C-10C, 12C-35C, 37C-90C, 91C, 93C-99C, 102C-105C, 108C, 111C, 113C, 114C, 116C-131C, and 133C-136C exhibited an intrinsic clearance of less than about 50 μL/min/1×10⁶ cells.

In Vitro Estimation of CYP1A2, CYP2B6 and CYP3A4 Induction Potention of Primary Human Hepatocytes

One objective of this assay is to investigate the potential for a test compound to modulate the expression of CYP1A2, CYP2B6, and CYP2A4 in plated cultures of cryopreserved human hepatocytes.

Materials.

Hepatocytes were purchased from commercial sources and stored at less than −150° C.

Preparation of Compound Working Solutions.

The stock solution of Test Compound (TC) and Positive Control chlorpromazine (PC) are prepared in DMSO at 1000× stock concentration and diluted to the respective working concentrations with 37° C. hepatocyte incubation medium. The final concentration of DMSO in the assay is 0.1%.

Seeding and Treatment of Hepatocytes

Vials of cryopreserved hepatocytes were removed from storage, depressurized, and thawed in a 37° C. water bath. The contents were poured into a 50 mL thawing medium conical tube containing hepatocyte thawing medium that had been pre-warmed to 37° C. Vials were centrifuged at 100 g for 10 minutes at room temperature. The thawing medium was aspirated and the hepatocytes were re-suspended with enough plating medium (pre-warmed at 37° C.) to yield ˜1.0×10⁶ cells/mL Viable cell density is determined and the cells are diluted with plating medium to a cell density of 0.55×10⁶ viable cells/mL. An aliquot (100 μL) of cells is transferred to each well of a collagen I coated 96-well plate. The plate is placed in an incubator for 4-5 hours (37° C., 5% CO₂, 95% relative humidity). After incubation, cell morphology and monolayer integrity are checked to confirm that cells are attached to the plate. The medium in the wells is replaced with 125 μL matrigel incubation medium having a concentration of 0.5-0.75 mg protein/mL. The plate is placed in the incubator for 18 hours. The plates are removed from the incubator and the medium is replaced with 125 μL of the 0.1% DMSO, TC solution or PC solution, each in triplicate. The TC solution or PC solution is renewed every 24 hours.

Determination of Cell Viability.

After 72 hours of treatment, the cell morphology is checked under microscope and a sample is removed to determine cell viability.

Enzyme Activity Assay.

Stock Solutions of the marker substrates phenacetin and midazolam are prepared in DMSO at 1000× for CYP1A2 and CYP3A4, respectively and bupropion in pure water at 100× for CYP2B6. Incubation medium pre-warmed at 37° C. is used to dilute the Stock Solution to a final concentration of phenacetin (CYP1A2) (100 μM), bupropion (CYP2B6) (500 μM), and midazolam (CYP3A4) (10 μM) to prepare substrate solution. The final concentration of DMSO in the substrate solution is ≤0.1%. Media is removed from the cell plates and the cell monolayers are washed with PBS and 125 μL of pre-warmed incubation modicum are added to the plates. The plates are incubated for 10 minutes (37° C., 5% CO₂, 95% relative humidity). After incubation, the contents of the wells of the plate are replaced with 125 μL of the probe substrate solutions. The plate(s) are incubated for 30 minutes (37° C., 5% CO₂, 95% relative humidity). At the end of the incubation period, an aliquot (100 μL) is removed directly from the wells to a new 96-well plate. The contents of the wells of the new plate are mixed with 400 μL quenching solution (acetonitrile with 0.5 μM tolbutamide), vortexed for 10 min, then centrifuged at 3,000×g, 4° C. for 30 min. An aliquot of 150 μL of the supernatant is mixed with 150 μL pure water for UPLC-MS/MS analysis. Marker metabolites acetaminophen, hydroxybupropion, and 1-hydroxymidazolam are diluted with Williams' E Medium to respective concentrations to prepare calibration curves. Quality control samples of the marker metabolites also are prepared. Metabolite stock solution (1 μL of 100×DMSO stock solution is added to 99 μL incubation medium and mixed with 400 μL of acetonitrile containing internal standard (0.5 μM tolbutamide) to prepare the samples for calibration curve and QC. The samples are centrifuged in the plate at 3000×g, 4° C. for 30 minutes to precipitate protein. A 150 μL sample of the supernatant is transferred to a new 96-well plate then 150 μL of pure water is added before analysis by UPLC-MS/MS. All incubations are conducted in triplicate. CYP activity is expressed as pmol/min/million cells where pmol is the amount of metabolite formed during the reaction. Fold induction of CYP activity for the TC versus vehicle is determined.

mRNA Level Assay

After removing the samples for the enzyme activity assay, mRNA is extracted using a commercially available kit The mRNA levels of CYP1A2, CYP2A6, and CYP3A4 are measured by performing quantitative fluorescent RT-PCR and mRNA content is expressed as the number of cycles required for the fluorescent signal to cross a selected threshold. Fold induction of CYP mRNA level for the TC versus control is determined.

In the above assay, compounds of Examples indicated herein were tested and fold induction levels for CYP3A4 mRNA were determined after the compounds were administered at a concentration of 1 μM. Examples 10, 2A, 3A, 10A-16A, 23A, 25A, 26A, 29A, 30A, 32A, 35A, 38A, 41A, 42A, 44A, 47A, 54A, 56A, 61A, 65A, 67A, 69A-71A, 83A, 104A-106A, 108A, 110A-112A, 114A, 116A, 120A, 123A, 126A, 128A, 132A, 137A, 140A, 147A, 2B, 5B-7B, 11B, 18B, 21B, 23B, 25B, 26B, 28B, 30B, 1C, 2C, 1C, 12C, 18C, 19C, 22C, 38C, 39C, 41C, 43C, 71C, 72C, 77C, 85C-90C, 92C-94C, 99C, 111C-113C, 115C, 117C, 118C, 121C, 125C, 126C, 128C, and 129C, exhibited a CYP3A4 mRNA fold induction level of less than about 3.0×.

1. A compound of the Formula: 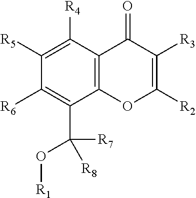
or pharmaceutically acceptable salt thereof, wherein:
R₁ is an optionally substituted bicyclic ring selected from isobenzofuranone, benzofuranone, isoindolinone, indolinone, quinazolinone, 3,4-dihydro-2H-isoquinolin-1-one, 2H-isoquinolin-1-one, or benzothiazolone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
R₁ is a group of the formula: 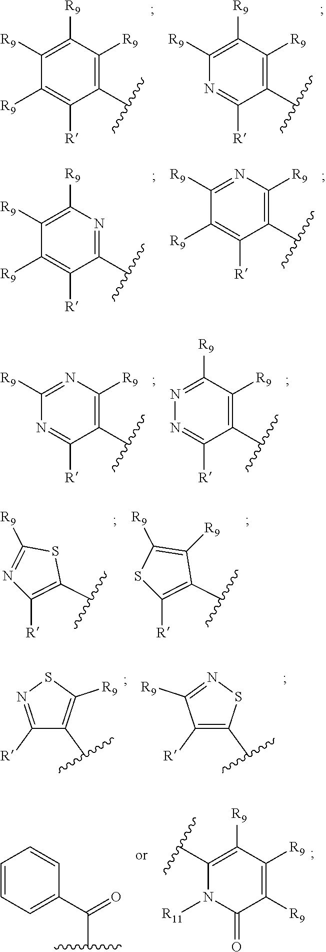
R′ is hydrogen, halogen, C₁-C₃ alkyl, C₁-C₃ haloalkyl, C₁-C₃ alkoxy, C₁-C₃ haloalkoxy, —OH, —CH(OH)—CH₂OH, —CO—CH₂OH, C₃-C₆ cycloalkyl, —NO₂, —N(R₁₁)—CO₂C₁-C₃ alkyl, —N(R₁₁)—SO₂C₁-C₃ alkyl, —N(R₁₁)—SO₂R₁₅, —SO₂C₁-C₃ alkyl, —SO₂NR₁₁R₁₁, —SO₂NR₁₁R₁₂, —SO₂N(R₁₁)—CO—C₁-C₃ alkyl, —SO₂N(R₁₁)—CN, —C(═N—OH)—NH₂, —CN, —CONR₁₁R₁₂, —CON(R₁₁)—(CH₂)ₙ—R₁₃, —CO—SR₁₂, or a group of the formula: 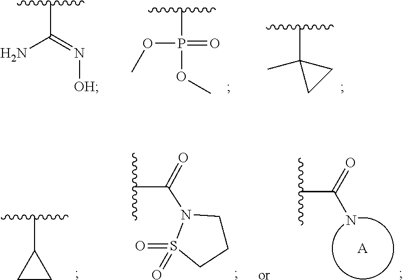
wherein ring A is pyrrolidine optionally substituted with a —CN; or
R′ is selected from oxetane, pyrrolidine, tetrahydrofuran, morpholine, piperidine, piperazine, pyrrole, furan, thiophene, pyrazole, imidazole, isoxazole, oxazole, isothiazole, thiazole, triazole, oxadiazole, thiadiazole, tetrazole, phenyl, pyridine, pyridazine, pyrimidine, pyrazine, or triazine; each of which is optionally substituted with one to three substituents independently selected from oxo, —OH, —NR₁₁R₁₁, —N(R₁₁)—CO—R₁₁, —N(R₁₁)—CN, —OR₁₁, —CN, halogen, C₁-C₆ haloalkyl or C₁-C₆alkyl optionally substituted with an aryl, a 5-member heteroaryl or a 6-member heteroaryl; or
R′ is a group of the formula: 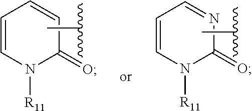
R₂ is a group of the formula: 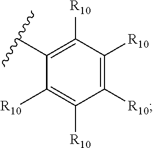
R₂ is a group of the formula: 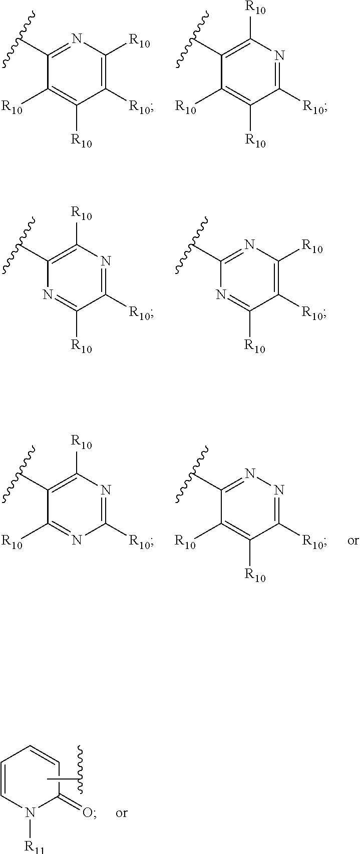
R₂ is an optionally substituted 5-member ring heteroaryl selected from pyrrole, furan, thiophene, pyrazole, isoxazole, isothiazole, imidazole, oxazole, thiazole, triazole, tetrazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —OH, —NR₁₁R₁₁, —NR₁₁CO₂R₁₁, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, halogen, oxetanyl, 2-oxabicyclo[2.1.1]hexane, C₁-C₃ alkoxy, an optionally substituted C₃-C₅ cycloalkyl, —CONR₁₁R₁₁, or —S(O)₂CH₃; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is an optionally substituted bicyclic ring selected from 1,3-benzodioxole, 2,3-dihydro-1,4-benzodioxine, quinoxaline, quinazoline, indole, indazole, isoindazole, benzimidazole, benzotriazole, benzothiazole, benzoxazole, benzotriazole, benzofuran, benzofurazan, pyridofurazan, quinoline, 1,5-naphthyridine, isoindolin-1-one, indolin-2-one, benzomorpholine, benzo[d]oxazol-2(3H)-one, imidazo[1,2-a]pyridine, 1,3-dihydro-2H-pyrrolo[2,3-b]pyridin-2-one, dioxolo[4,5-b]pyridine, 6,7-dihydro-4H-pyrazolo[5,1-c][1,4]oxazine, 1,2,3,4-tetrahydroisoquinoline, 4,5,6,7-tetrahydropyrazolo[1,5-a]pyrazine, pyrazolo[4,3-b]pyridine, pyrazolo[3,4-b]pyridine, pyrazolo[3,4-c]pyridine, pyrazolo[1,5-a]pyrimidine, oxazolo[4,5-b]pyridine, oxazolo[5,4-b]pyridine, thiazolo[5,4-b]pyridine, 2,1,3-benzothiadiazole, imidazo[1,2-a]pyrimidine, 2,3-dihydro-[1,4]dioxino[2,3-b]pyridine, 6,7-dihydro-5H-cyclopent[b]pyridine, 5,6-dihydro-4H-pyrrolo[1,2-b]pyrazole, 3,4-dihydro-2(1H)quinolinone, 2H-1,4-benzoxazin-3(4H)-one, 2-hydroxyquinoline, 3,4-dihydroisoquinolin-1,(2H)-one, 1-hydroxyisoquinoline, 1,4-dihydro-3(2H)-isoquinolinone, 3H-imidazo[4,5-b]pyridine, 4,5-dihydro-7H-pyrazolo[1,5-c]1,3]oxazine, 6,7-dihydro-5H-pyrazolo[5,1-b][1,3]oxazine, furo[3,2-c]pyridine, furo[3,2-b]pyridine, 2,3-dihydropyrazolo[5,1-b]oxazole, 5H,6H,8H-imidazo[2,1-c][1,4]oxazine, pyrazolo[1,5-b]pyridazine, imidazo[1,2-b]pyridazine, 2,4-dihydro-1H-benzo[d][1,3]oxazine, 5-oxaspiro[2,3]hexane, imidazo[1,5-a]pyridine, or pyrazolo[1,5-a]pyridine; or an optionally substituted bicyclic heteroaryl of 8 to 10 ring atoms containing 1, 2, 3, 4, or 5 ring heteroatoms independently selected from N, O, or S; wherein the optionally substituted bicyclic ring or the optionally substituted bicyclic heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —NR₁₁R₁₁, —NR₁₁CO₂R₁₁, —NR₁₁C(O)R₁₁, —OH, oxetanyl, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, —CONR₁₁R₁₁, phenyl, or an optionally substituted C₃-C₅ cycloalkyl; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is a cyclopropyl, cyclobutyl, cyclopentyl, or cyclohexyl, each optionally substituted with one to three R₁₀ substituents, or R₂ is a group of the formula: 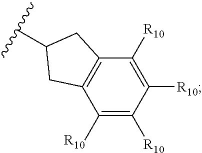
R₃ is —H, halogen, —CN, —N(H)(C₁-C₃ alkyl), —N(C₁-C₃ alkyl)₂, —N(H)(CH₂CH₂CO₂H), —CO—C₁-C₃ alkyl, C₁-C₆ alkyl, C₁-C₆ haloalkyl, C₁-C₆ hydroxyalkyl, C₃-C₅ cycloalkyl, an optionally substituted heterocycle of 3 to 5 ring atoms containing 1, 2, or 3 ring heteroatoms independently selected from N, O, or S, or an optionally substituted heteroaryl of 5 or 6 ring atoms containing 1, 2, or 3 ring heteroatoms independently selected from N, O, or S; wherein the optionally substituted heterocycle or heteroaryl is each optionally substituted with one to three substituents each independently selected from halogen, C₁-C₃ alkyl, or C₁-C₃ haloalkyl;
each of R₄, R₅ and R₆ is independently —H, halogen, C₁-C₆ alkyl or C₁-C₆ haloalkyl;
R₇ is —CN, C₁-C₆ alkyl or C₁-C₆ haloalkyl;
R₈ is —H or C₁-C₆ alkyl;
each R₉ is independently —H, halogen, —CN, C₁-C₆ alkyl, C₁-C₆ haloalkyl, C₁-C₆ alkoxy, or C₃-C₅ cycloalkyl;
each R₁₀ is independently —H, —CN, halogen, C₁-C₆ haloalkyl, C₁-C₆ alkoxy, C₁-C₆ haloalkoxy, —SO₂R₁₁, —SONR₁₁R₁₁, —CO₂H, —CO₂C₁-C₃ alkyl, —CONR₁₁R₁₂, —NR₁₁R₁₁, —NR₁₁—CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, an optionally substituted heteroaryl selected from pyrazole, isoxazole, isothiazole, imidazole, oxazole, thiazole, or pyridine, or a group of the formula: 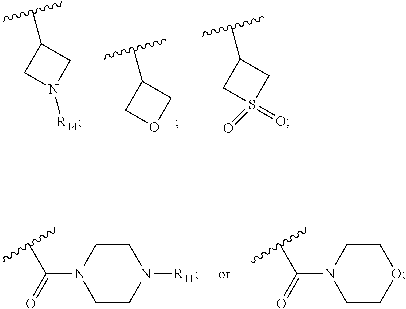
wherein the optionally substituted C₁-C₆ alkyl, C₂-C₆ alkenyl, or C2-C₆ alkynyl is each optionally substituted with a —CN, —OH, oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN;
each R₁₁ is independently —H or C₁-C₃ alkyl;
each R₁₂ is independently —H, optionally substituted C₁-C₃ alkyl, C₃-C₆ cycloalkyl, —SO₂C₁-C₃ alkyl, —SO₂C₁-C₃ haloalkyl, —SO₂NR₁₁R₁₁, —NR₁₁R₁₁, —OR₁₁, —O—CH₂—CH(OH)—CH₂OH, —CN, oxetane, tetrahydrofuran, an aryl, a 5-member heteroaryl optionally substituted with a methyl, a 6-member heteroaryl, or a group of the formula 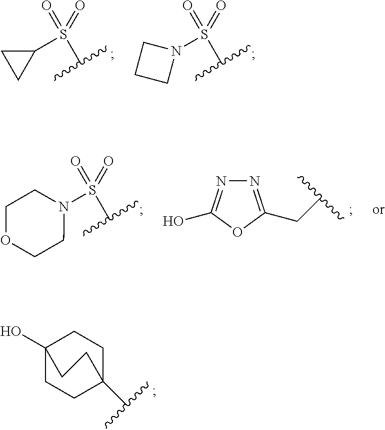 [SMILES: *S(=O)(=O)C1CC1 ; *S(=O)(=O)N1CCC1 ; *S(=O)(=O)N1CCOCC1 ; *Cc1nnc(O)o1 ; *C12CCC(O)(CC1)CC2]
wherein the optionally substituted C₁-C₃ alkyl is optionally substituted with —OH, a C₃-C₆ cycloalkyl, oxetane, tetrahydrofuran, an aryl, a 5-member heteroaryl, a 6-member heteroaryl or an indole;
R₁₃ is —NR₁₁R₁₁, —OR₁₁, —SO₂C₁-C₃ alkyl, or a ring selected from oxetane, tetrahydrofuran or oxadiazole wherein the ring is optionally substituted with —NR₁₁R₁₁, or —OR₁₁;
R₁₄ is —H, optionally substituted C₁-C₃ alkyl, —SO₂C₁-C₃ alkyl, an aryl, a 5-member heteroaryl, or a 6-member heteroaryl; wherein the optionally substituted C₁-C₃ alkyl is optionally substituted with an aryl, a 5-member heteroaryl or a 6-member heteroaryl;
R₁₅ is an optionally substituted aryl, or an optionally substituted 6-member heteroaryl; wherein the optionally substituted aryl, or the optionally substituted 6-member heteroaryl is each optionally substituted with one to three substituents each independently selected from halogen, C₁-C₃ alkyl, or C₁-C₃ haloalkyl; and
n is 0, 1 or 2.

2. The compound of claim 1 or pharmaceutically acceptable salt thereof, wherein:
R₁ is an optionally substituted bicyclic ring selected from isobenzofuranone, benzofuranone, isoindolinone, indolinone, quinazolinone, 3,4-dihydro-2H-isoquinolin-1-one, 2H-isoquinolin-1-one, or benzothiazolone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
R₁ is a group of the formula: 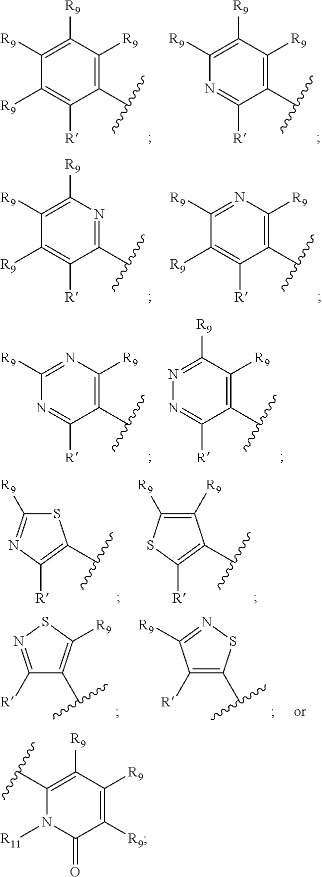
R′ is hydrogen, halogen, C₁-C₃ alkyl, C₁-C₃ haloalkyl, C₁-C₃ alkoxy, C₁-C₃ haloalkoxy, —OH, —CH(OH)—CH₂OH, —CO—CH₂OH, C₃-C₆ cycloalkyl, —NO₂, —N(R₁₁)—CO₂C₁-C₃ alkyl, —N(R₁₁)—SO₂C₁-C₃ alkyl, —N(R₁₁)—SO₂R₁₅, —SO₂C₁-C₃ alkyl, —SO₂NR₁₁R₁₁, —SO₂N(R₁₁)—CO—C₁-C₃ alkyl, —SO₂N(R₁₁)—CN, —C(═N—OH)—NH₂, —CN, —CONR₁₁R₁₂, —CON(R₁₁)—(CH₂)ₙ—R₁₃, —CO—SR₁₂, or a group of the formula: 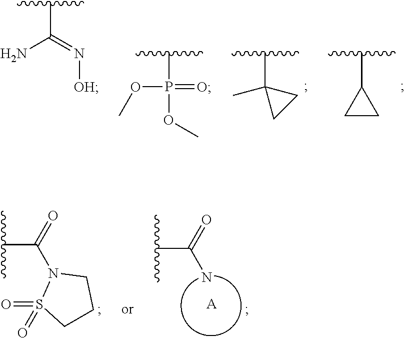
wherein ring A is pyrrolidine optionally substituted with a —CN; or
R′ is selected from oxetane, pyrrolidine, tetrahydrofuran, morpholine, piperidine, piperazine, pyrrole, furan, thiophene, pyrazole, imidazole, isoxazole, oxazole, isothiazole, thiazole, triazole, oxadiazole, thiadiazole, tetrazole, phenyl, pyridine, pyridazine, pyrimidine, pyrazine, or triazine; each of which is optionally substituted with one to three substituents independently selected from oxo, —OH, —NR₁₁R₁₁, —N(R₁₁)—CO—R₁₁, —N(R₁₁)—CN, —OR₁₁, —CN, halogen, C₁-C₆ haloalkyl or C₁-C₆alkyl optionally substituted with an aryl, a 5-member heteroaryl or a 6-member heteroaryl; or
R′ is a group of the formula: 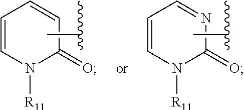
R₂ is a group of the formula: 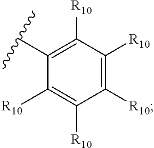
or
R₂ is a group of the formula: 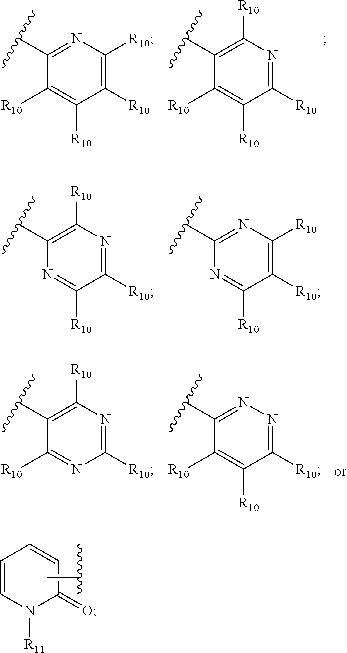
or
R₂ is an optionally substituted 5-member ring heteroaryl selected from pyrrole, furan, thiophene, pyrazole, isoxazole, isothiazole, imidazole, oxazole, thiazole, triazole, tetrazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —OH, —NR₁₁R₁₁, —NR₁₁CO₂R₁₁, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is an optionally substituted bicyclic ring selected from 1,3-benzodioxole, 2,3-dihydro-1,4-benzodioxine, isoindolin-1-one, indolin-2-one, benzo[d]oxazol-2(3H)-one, 1,3-dihydro-2H-pyrrolo[2,3-b]pyridin-2-one, or 2,3-dihydro-[1,4]dioxino[2,3-b]pyridine, or an optionally substituted bicyclic heteroaryl of 8 to 10 ring atoms containing 1, 2, 3, 4, or 5 ring heteroatoms independently selected from N, O, or S; wherein the optionally substituted bicyclic ring is optionally substituted with one to three substituents each independently selected from halogen and C₁-C₆ alkyl; the optionally substituted bicyclic heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —NR₁₁R₁₁, —NR₁₁CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH (e.g., C₁-C₆ hydroxyalkyl such as —CH₂—CH₂—OH and optionally comprising a secondary —OH such as —CH₂—CH(CH₃)—OH or a tertiary —OH such as —CH₂—C(CH₃)₂—OH), oxetanyl, C₁-C₃ alkoxy, —CONR₁₁R₁₁ or phenyl; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is a cyclopropyl, cyclobutyl, cyclopentyl, or cyclohexyl, each optionally substituted with one to three R₁₀ substituents, or R₂ is a group of the formula: 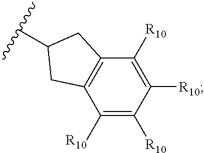
R₃ is —H, halogen, —CN, —N(H)(C₁-C₃ alkyl), —N(C₁-C₃ alkyl)₂, —N(H)(CH₂CH₂CO₂H), —CO—C₁-C₃ alkyl, C₁-C₆ alkyl, C₁-C₆ haloalkyl, C₁-C₆ hydroxyalkyl, C₃-C₅ cycloalkyl, an optionally substituted heterocycle of 3 to 5 ring atoms containing 1, 2, or 3 ring heteroatoms independently selected from N, O, or S, or an optionally substituted heteroaryl of 5 or 6 ring atoms containing 1, 2, or 3 ring heteroatoms independently selected from N, O, or S; wherein the optionally substituted heterocycle or heteroaryl is each optionally substituted with one to three substituents each independently selected from halogen, C₁-C₃ alkyl, or C₁-C₃ haloalkyl;
each R₁₀ is independently —H, —CN, halogen, C₁-C₆ haloalkyl, C₁-C₆ alkoxy, C₁-C₆ haloalkoxy, —SO₂R₁₁, —SONR₁₁R₁₁, —CO₂H, —CO₂C₁-C₃ alkyl, —CONR₁₁R₁₂, —NR₁₁R₁₁, —NR₁₁—CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, an optionally substituted heteroaryl selected from pyrazole, isoxazole, isothiazole, imidazole, oxazole, thiazole, or pyridine, or a group of the formula: 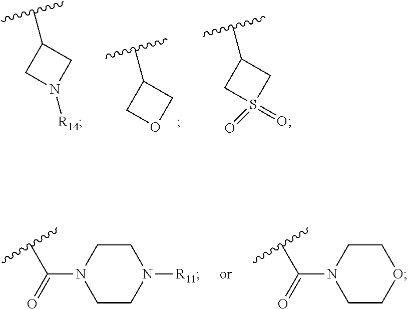
wherein the optionally substituted C₁-C₆ alkyl, C₂-C₆ alkenyl, or C2-C₆ alkynyl is each optionally substituted with a —CN, —OH, oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN;
each R₁₁ is independently —H or C₁-C₃ alkyl;
each R₁₂ is independently —H, optionally substituted C₁-C₃ alkyl, C₃-C₆ cycloalkyl, —SO₂C₁-C₃ alkyl, —SO₂C₁-C₃ haloalkyl, —SO₂NR₁₁R₁₁, —NR₁₁R₁₁, —OR₁₁, —O—CH₂—CH(OH)—CH₂OH, —CN, oxetane, tetrahydrofuran, an aryl, a 5-member heteroaryl optionally substituted with a methyl, a 6-member heteroaryl, or a group of the formula 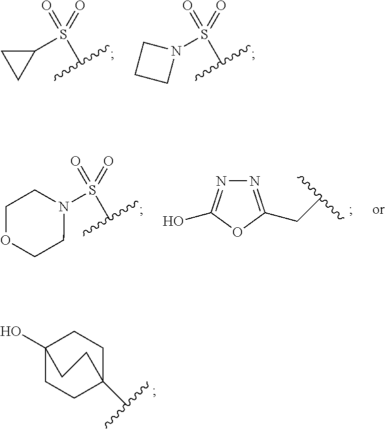 [SMILES: *S(=O)(=O)C1CC1 ; *S(=O)(=O)N1CCC1 ; *S(=O)(=O)N1CCOCC1 ; *Cc1nnc(O)o1 ; *C12CCC(O)(CC1)CC2]
wherein the optionally substituted C₁-C₃ alkyl is optionally substituted with —OH, a C₃-C₆ cycloalkyl, oxetane, tetrahydrofuran, an aryl, a 5-member heteroaryl, a 6-member heteroaryl or an indole;
R₁₃ is —NR₁₁R₁₁, —OR₁₁, —SO₂C₁-C₃ alkyl, or a ring selected from oxetane, tetrahydrofuran or oxadiazole wherein the ring is optionally substituted with —NR₁₁R₁₁, or —OR₁₁;
R₁₄ is —H, optionally substituted C₁-C₃ alkyl, —SO₂C₁-C₃ alkyl, an aryl, a 5-member heteroaryl, or a 6-member heteroaryl; wherein the optionally substituted C₁-C₃ alkyl is optionally substituted with an aryl, a 5-member heteroaryl or a 6-member heteroaryl;
R₁₅ is an optionally substituted aryl, or an optionally substituted 6-member heteroaryl; wherein the optionally substituted aryl, or the optionally substituted 6-member heteroaryl is each optionally substituted with one to three substituents each independently selected from halogen, C₁-C₃ alkyl, or C₁-C₃ haloalkyl; and
n is 0, 1 or 2.

3. (canceled)

4. The compound of claim 1, or pharmaceutically acceptable salt thereof, having the Formula:

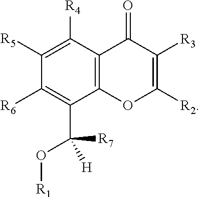

5.-15. (canceled)

16. The compound of claim 1, or pharmaceutically acceptable salt thereof having the Formula

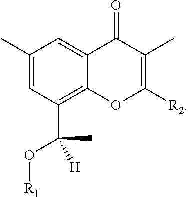

17. The compound of claim 1, or pharmaceutically acceptable salt thereof, wherein R₁ is a group of the formula

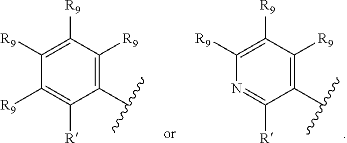

18. The compound of claim 1, or pharmaceutically acceptable salt thereof, wherein each R₉ is independently —H, or halogen.

19. The compound of claim 1, or pharmaceutically acceptable salt thereof, wherein R′ is hydrogen, C₁-C₃ alkyl, —CONH—SO₂C₁-C₃ alkyl, or oxadiazolinone.

20. The compound of claim 1, or pharmaceutically acceptable salt thereof, wherein R′ is —SO₂NR₁₁R₁₁, —SO₂N(R₁₁)(R₁₃), —C(O)NR₁₁R₁₂, —C(O)N(R₁₁)—(CH₂)ₙ—R₁₃, or —C(O)—NHSO₂R₁₆.

21. The compound of claim 1, or pharmaceutically acceptable salt thereof, wherein R₂ is bound to the chromenone core of the carbon via a ring carbon of a cycloalkyl group, via a ring carbon of a heterocycloalkyl group, via a ring carbon of an aryl group, or via a ring carbon of a heteroaryl group.

22. The compound of claim 1, or pharmaceutically acceptable salt thereof, wherein R₂ is a group of the formula: 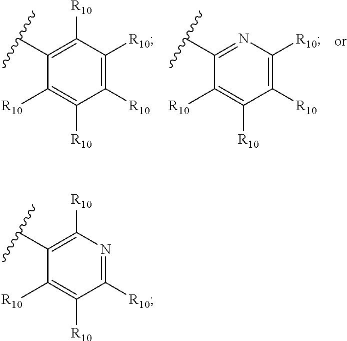
wherein each R₁₀ is independently —H, —CN, halogen, C₁-C₆ alkyl, or C₁-C₆ haloalkyl; or R₂ is an optionally substituted indazole, or an optionally substituted pyrazole, each of which is optionally substituted with one to three substituents each independently selected from —CN, halogen, C₁-C₆ alkyl, or C₁-C₆ haloalkyl.

23. The compound of claim 1, or pharmaceutically acceptable salt thereof, wherein R₂ is a group of the formula: 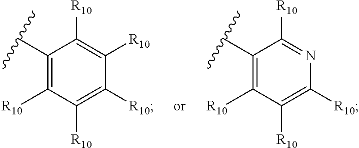
wherein each R₁₀ is independently —H, —CN, halogen, C₁-C₆ alkyl, or C₁-C₆ haloalkyl.

24. The compound of claim 1, or pharmaceutically acceptable salt thereof, wherein R₂ is 1H-indazol-5-yl, 1H-indazol-6-yl, or 1H-pyrazol-4-yl, which is N-substituted with C₁-C₃ alkyl or C₁-C₆ hydroxyalkyl.

25. The compound of claim 1, or pharmaceutically acceptable salt thereof, selected from:

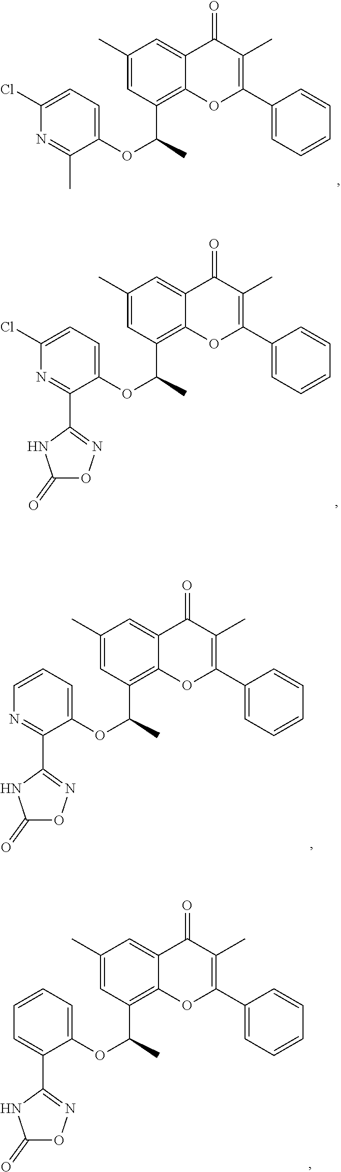 [SMILES: Cc1cc([C@@H](C)Oc2ccc(Cl)nc2C)c2oc(-c3ccccc3)c(C)c(=O)c2c1 ; Cc1cc([C@@H](C)Oc2ccc(Cl)nc2-c2noc(=O)[nH]2)c2oc(-c3ccccc3)c(C)c(=O)c2c1 ; Cc1cc([C@@H](C)Oc2cccnc2-c2noc(=O)[nH]2)c2oc(-c3ccccc3)c(C)c(=O)c2c1 ; Cc1cc([C@@H](C)Oc2ccccc2-c2noc(=O)[nH]2)c2oc(-c3ccccc3)c(C)c(=O)c2c1]

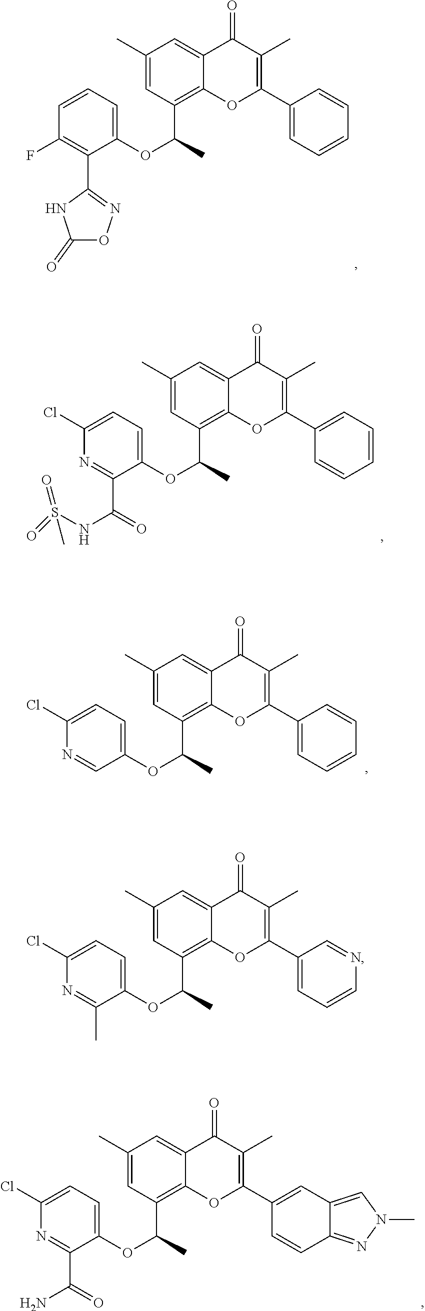 [SMILES: Cc1cc([C@@H](C)Oc2cccc(F)c2-c2noc(=O)[nH]2)c2oc(-c3ccccc3)c(C)c(=O)c2c1 ; Cc1cc([C@@H](C)Oc2ccc(Cl)nc2C(=O)NS(C)(=O)=O)c2oc(-c3ccccc3)c(C)c(=O)c2c1 ; Cc1cc([C@@H](C)Oc2ccc(Cl)nc2)c2oc(-c3ccccc3)c(C)c(=O)c2c1 ; Cc1cc([C@@H](C)Oc2ccc(Cl)nc2C)c2oc(-c3cccnc3)c(C)c(=O)c2c1 ; Cc1cc([C@@H](C)Oc2ccc(Cl)nc2C(N)=O)c2oc(-c3ccc4nn(C)cc4c3)c(C)c(=O)c2c1]

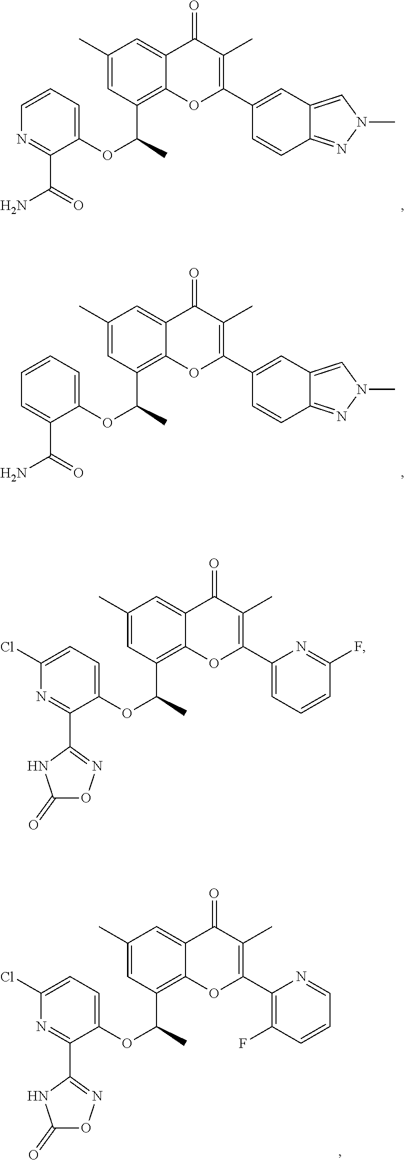 [SMILES: Cc1cc([C@@H](C)Oc2cccnc2C(N)=O)c2oc(-c3ccc4nn(C)cc4c3)c(C)c(=O)c2c1 ; Cc1cc([C@@H](C)Oc2ccccc2C(N)=O)c2oc(-c3ccc4nn(C)cc4c3)c(C)c(=O)c2c1 ; Cc1cc([C@@H](C)Oc2ccc(Cl)nc2-c2noc(=O)[nH]2)c2oc(-c3cccc(F)n3)c(C)c(=O)c2c1 ; Cc1cc([C@@H](C)Oc2ccc(Cl)nc2-c2noc(=O)[nH]2)c2oc(-c3ncccc3F)c(C)c(=O)c2c1]

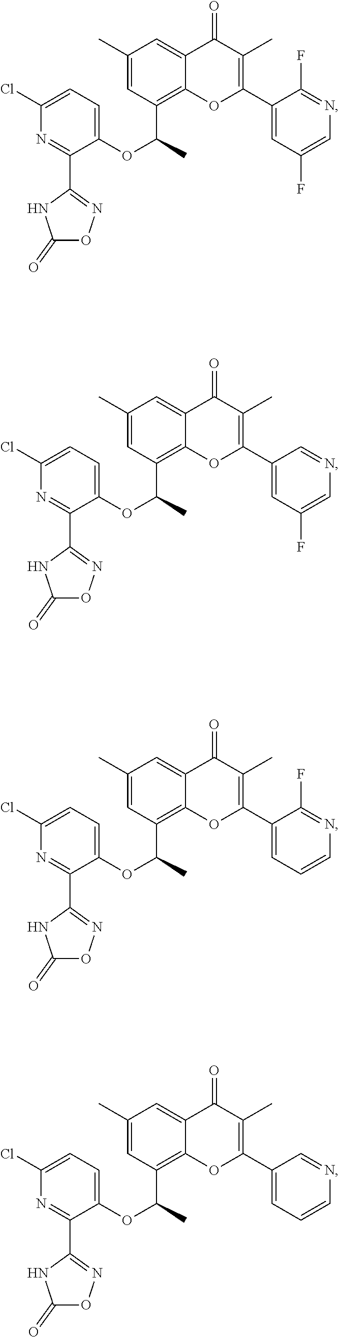 [SMILES: Cc1cc([C@@H](C)Oc2ccc(Cl)nc2-c2noc(=O)[nH]2)c2oc(-c3cc(F)cnc3F)c(C)c(=O)c2c1 ; Cc1cc([C@@H](C)Oc2ccc(Cl)nc2-c2noc(=O)[nH]2)c2oc(-c3cncc(F)c3)c(C)c(=O)c2c1 ; Cc1cc([C@@H](C)Oc2ccc(Cl)nc2-c2noc(=O)[nH]2)c2oc(-c3cccnc3F)c(C)c(=O)c2c1 ; Cc1cc([C@@H](C)Oc2ccc(Cl)nc2-c2noc(=O)[nH]2)c2oc(-c3cccnc3)c(C)c(=O)c2c1]

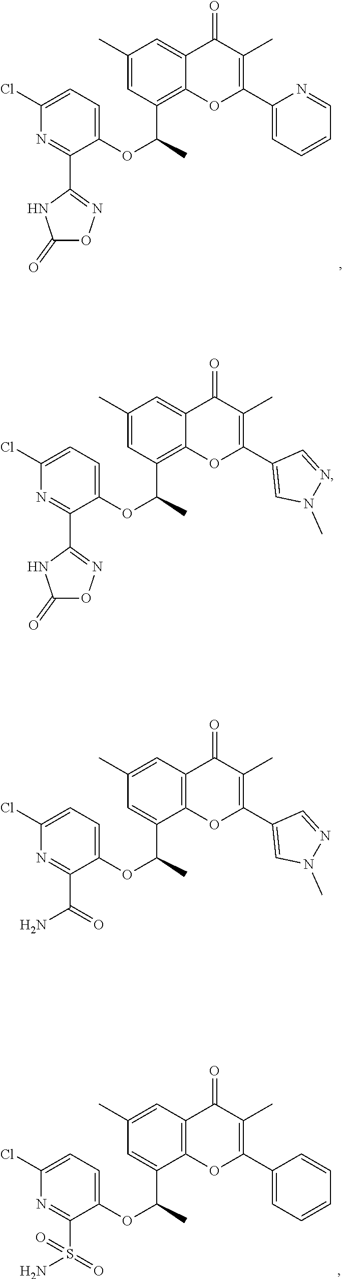 [SMILES: Cc1cc([C@@H](C)Oc2ccc(Cl)nc2-c2noc(=O)[nH]2)c2oc(-c3ccccn3)c(C)c(=O)c2c1 ; Cc1cc([C@@H](C)Oc2ccc(Cl)nc2-c2noc(=O)[nH]2)c2oc(-c3cnn(C)c3)c(C)c(=O)c2c1 ; Cc1cc([C@@H](C)Oc2ccc(Cl)nc2C(N)=O)c2oc(-c3cnn(C)c3)c(C)c(=O)c2c1 ; Cc1cc([C@@H](C)Oc2ccc(Cl)nc2S(N)(=O)=O)c2oc(-c3ccccc3)c(C)c(=O)c2c1]

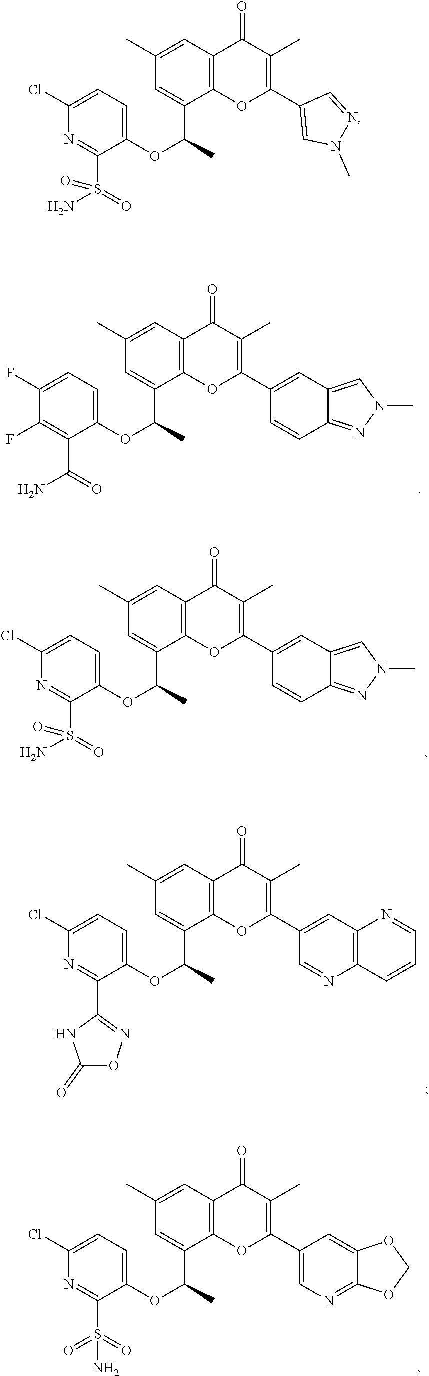 [SMILES: Cc1cc([C@@H](C)Oc2ccc(Cl)nc2S(N)(=O)=O)c2oc(-c3cnn(C)c3)c(C)c(=O)c2c1 ; Cc1cc([C@@H](C)Oc2ccc(F)c(F)c2C(N)=O)c2oc(-c3ccc4nn(C)cc4c3)c(C)c(=O)c2c1 ; Cc1cc([C@@H](C)Oc2ccc(Cl)nc2S(N)(=O)=O)c2oc(-c3ccc4nn(C)cc4c3)c(C)c(=O)c2c1 ; Cc1cc([C@@H](C)Oc2ccc(Cl)nc2-c2noc(=O)[nH]2)c2oc(-c3cnc4cccnc4c3)c(C)c(=O)c2c1 ; Cc1cc([C@@H](C)Oc2ccc(Cl)nc2S(N)(=O)=O)c2oc(-c3cnc4c(c3)OCO4)c(C)c(=O)c2c1]

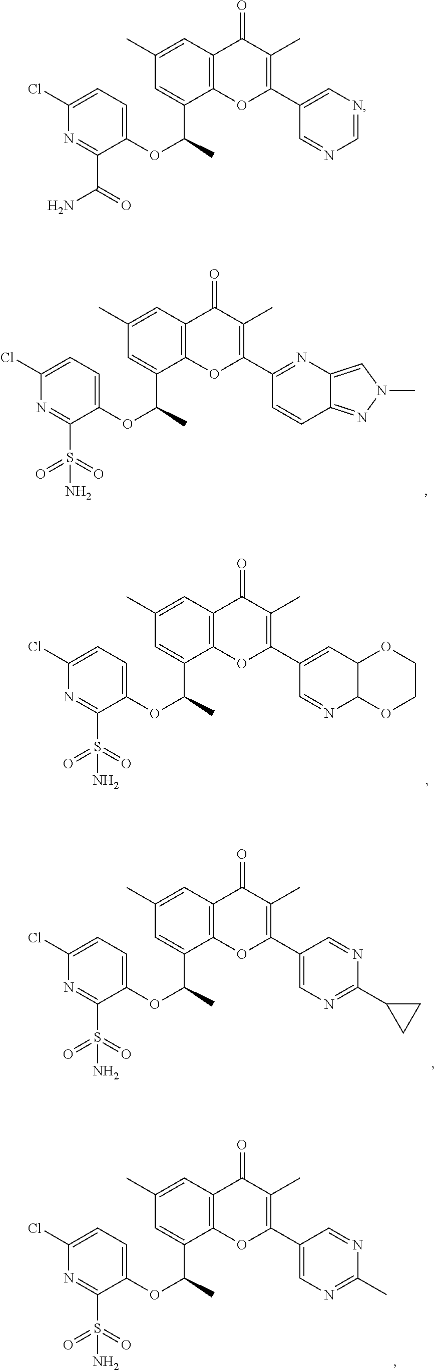 [SMILES: Cc1cc([C@@H](C)Oc2ccc(Cl)nc2C(N)=O)c2oc(-c3cncnc3)c(C)c(=O)c2c1 ; Cc1cc([C@@H](C)Oc2ccc(Cl)nc2S(N)(=O)=O)c2oc(-c3ccc4nn(C)cc4n3)c(C)c(=O)c2c1 ; Cc1cc([C@@H](C)Oc2ccc(Cl)nc2S(N)(=O)=O)c2oc(C3=CC4OCCOC4N=C3)c(C)c(=O)c2c1 ; Cc1cc([C@@H](C)Oc2ccc(Cl)nc2S(N)(=O)=O)c2oc(-c3cnc(C4CC4)nc3)c(C)c(=O)c2c1 ; Cc1cc([C@@H](C)Oc2ccc(Cl)nc2S(N)(=O)=O)c2oc(-c3cnc(C)nc3)c(C)c(=O)c2c1]

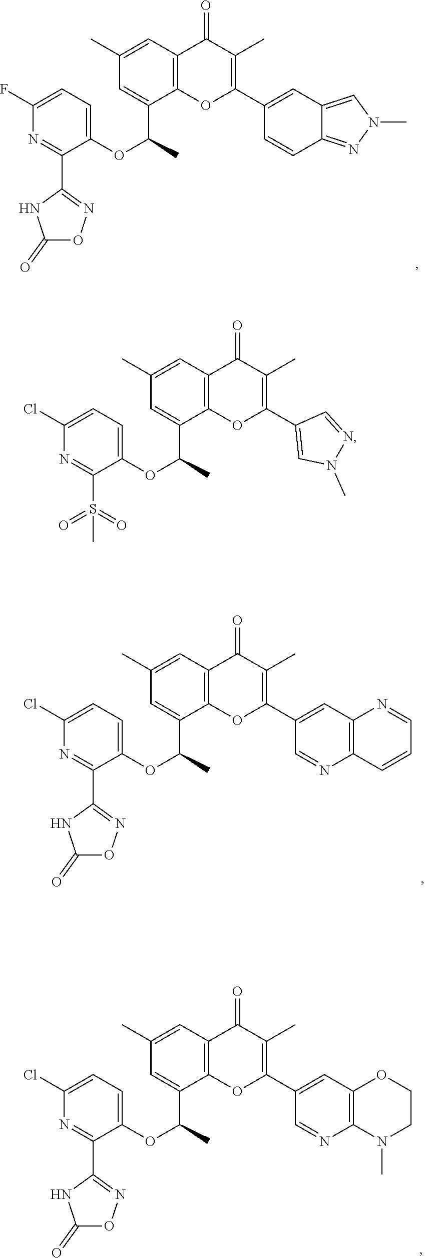 [SMILES: Cc1cc([C@@H](C)Oc2ccc(F)nc2-c2noc(=O)[nH]2)c2oc(-c3ccc4nn(C)cc4c3)c(C)c(=O)c2c1 ; Cc1cc([C@@H](C)Oc2ccc(Cl)nc2S(C)(=O)=O)c2oc(-c3cnn(C)c3)c(C)c(=O)c2c1 ; Cc1cc([C@@H](C)Oc2ccc(Cl)nc2-c2noc(=O)[nH]2)c2oc(-c3cnc4cccnc4c3)c(C)c(=O)c2c1 ; Cc1cc([C@@H](C)Oc2ccc(Cl)nc2-c2noc(=O)[nH]2)c2oc(-c3cnc4c(c3)OCCN4C)c(C)c(=O)c2c1]

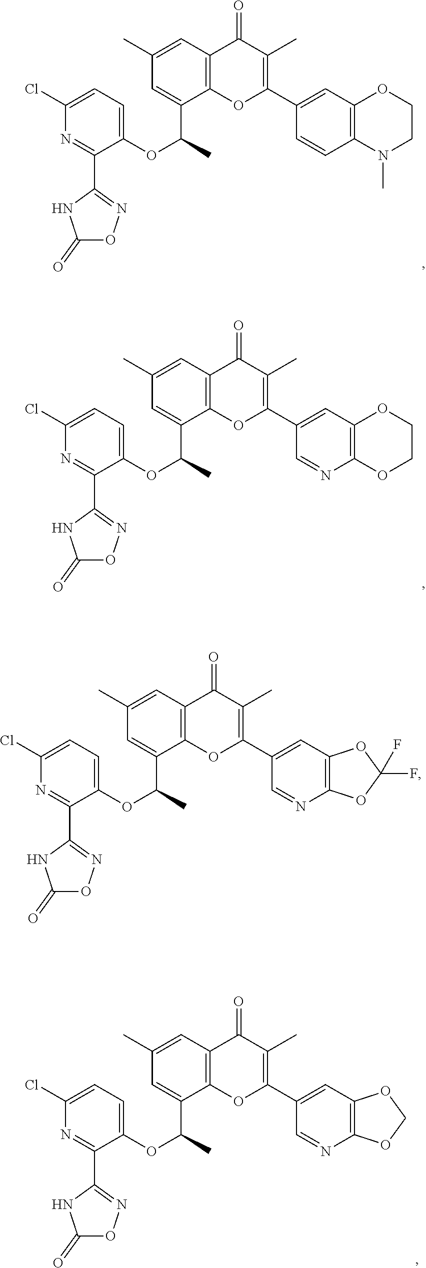 [SMILES: Cc1cc([C@@H](C)Oc2ccc(Cl)nc2-c2noc(=O)[nH]2)c2oc(-c3ccc4c(c3)OCCN4C)c(C)c(=O)c2c1 ; Cc1cc([C@@H](C)Oc2ccc(Cl)nc2-c2noc(=O)[nH]2)c2oc(-c3cnc4c(c3)OCCO4)c(C)c(=O)c2c1 ; Cc1cc([C@@H](C)Oc2ccc(Cl)nc2-c2noc(=O)[nH]2)c2oc(-c3cnc4c(c3)OC(F)(F)O4)c(C)c(=O)c2c1 ; Cc1cc([C@@H](C)Oc2ccc(Cl)nc2-c2noc(=O)[nH]2)c2oc(-c3cnc4c(c3)OCO4)c(C)c(=O)c2c1]

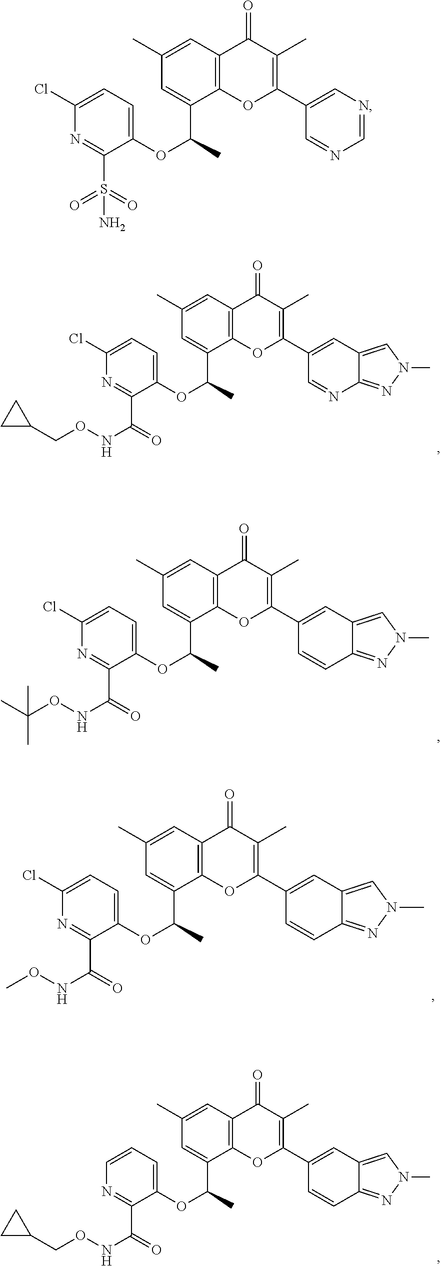

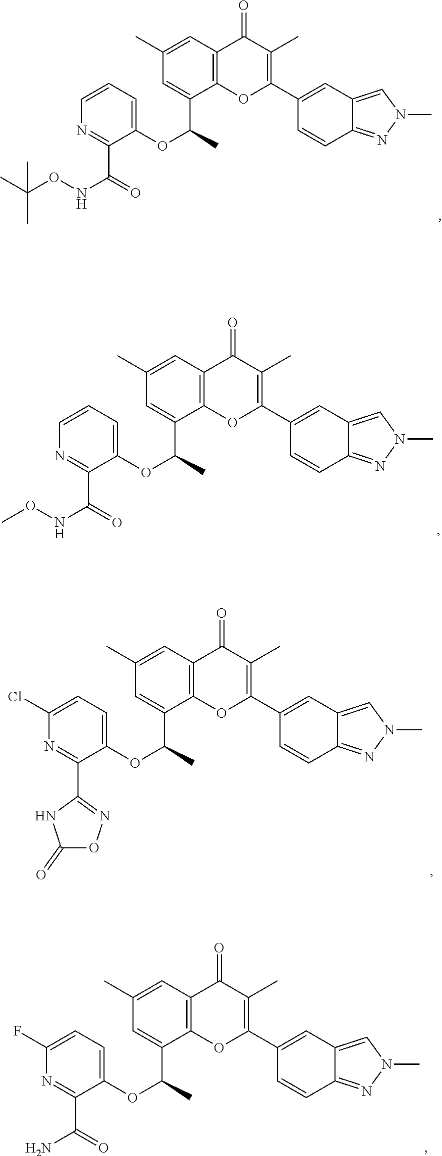

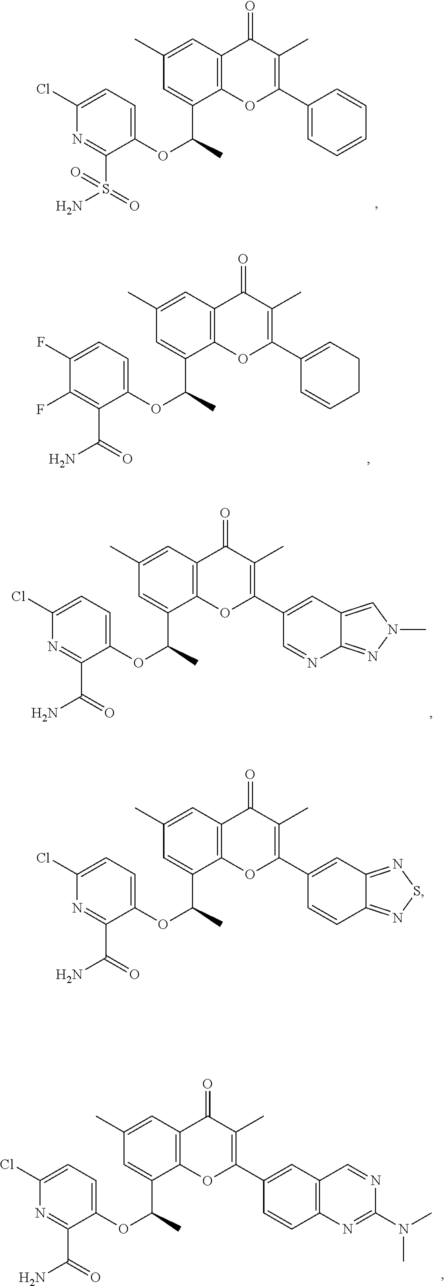 [SMILES: Cc1cc([C@@H](C)Oc2ccc(Cl)nc2S(N)(=O)=O)c2oc(-c3ccccc3)c(C)c(=O)c2c1 ; Cc1cc([C@@H](C)Oc2ccc(F)c(F)c2C(N)=O)c2oc(C3=CCCC=C3)c(C)c(=O)c2c1 ; Cc1cc([C@@H](C)Oc2ccc(Cl)nc2C(N)=O)c2oc(-c3cnc4nn(C)cc4c3)c(C)c(=O)c2c1 ; Cc1cc([C@@H](C)Oc2ccc(Cl)nc2C(N)=O)c2oc(-c3ccc4nsnc4c3)c(C)c(=O)c2c1 ; Cc1cc([C@@H](C)Oc2ccc(Cl)nc2C(N)=O)c2oc(-c3ccc4nc(N(C)C)ncc4c3)c(C)c(=O)c2c1]

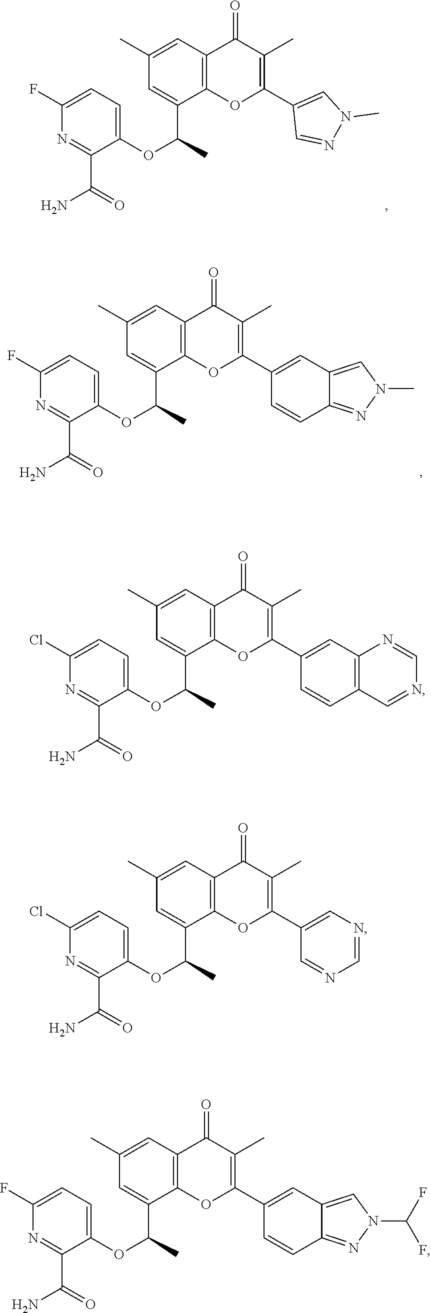 [SMILES: Cc1cc([C@@H](C)Oc2ccc(F)nc2C(N)=O)c2oc(-c3cnn(C)c3)c(C)c(=O)c2c1 ; Cc1cc([C@@H](C)Oc2ccc(F)nc2C(N)=O)c2oc(-c3ccc4nn(C)cc4c3)c(C)c(=O)c2c1 ; Cc1cc([C@@H](C)Oc2ccc(Cl)nc2C(N)=O)c2oc(-c3ccc4cncnc4c3)c(C)c(=O)c2c1 ; Cc1cc([C@@H](C)Oc2ccc(Cl)nc2C(N)=O)c2oc(-c3cncnc3)c(C)c(=O)c2c1 ; Cc1cc([C@@H](C)Oc2ccc(F)nc2C(N)=O)c2oc(-c3ccc4nn(C(F)F)cc4c3)c(C)c(=O)c2c1]

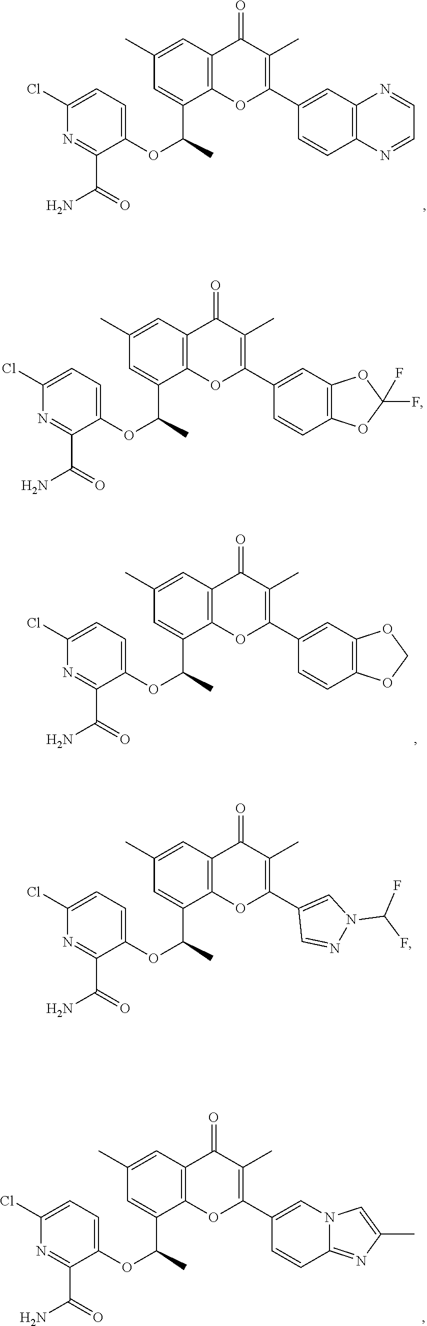 [SMILES: Cc1cc([C@@H](C)Oc2ccc(Cl)nc2C(N)=O)c2oc(-c3ccc4nccnc4c3)c(C)c(=O)c2c1 ; Cc1cc([C@@H](C)Oc2ccc(Cl)nc2C(N)=O)c2oc(-c3ccc4c(c3)OC(F)(F)O4)c(C)c(=O)c2c1 ; Cc1cc([C@@H](C)Oc2ccc(Cl)nc2C(N)=O)c2oc(-c3ccc4c(c3)OCO4)c(C)c(=O)c2c1 ; Cc1cc([C@@H](C)Oc2ccc(Cl)nc2C(N)=O)c2oc(-c3cnn(C(F)F)c3)c(C)c(=O)c2c1 ; Cc1cc([C@@H](C)Oc2ccc(Cl)nc2C(N)=O)c2oc(-c3ccc4nc(C)cn4c3)c(C)c(=O)c2c1]

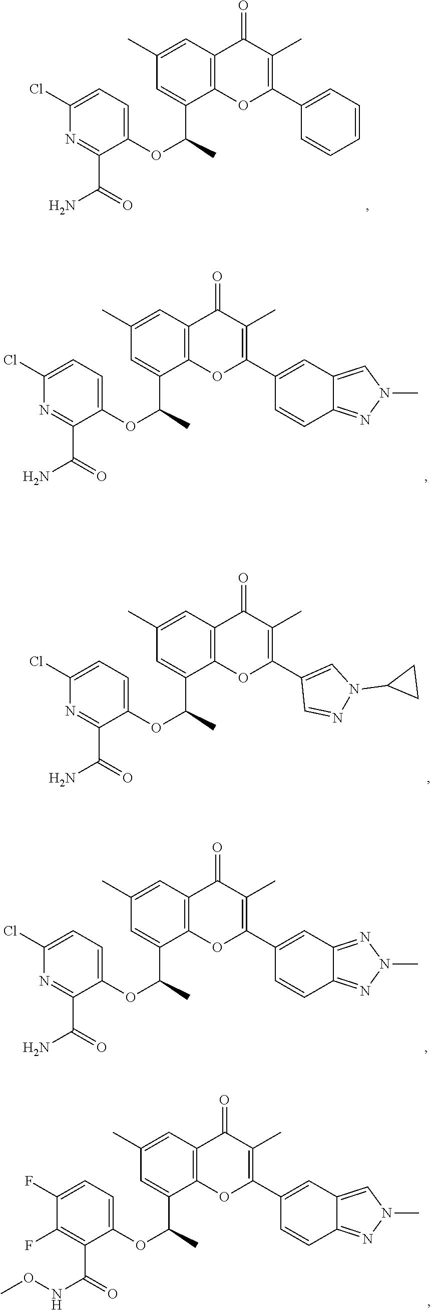

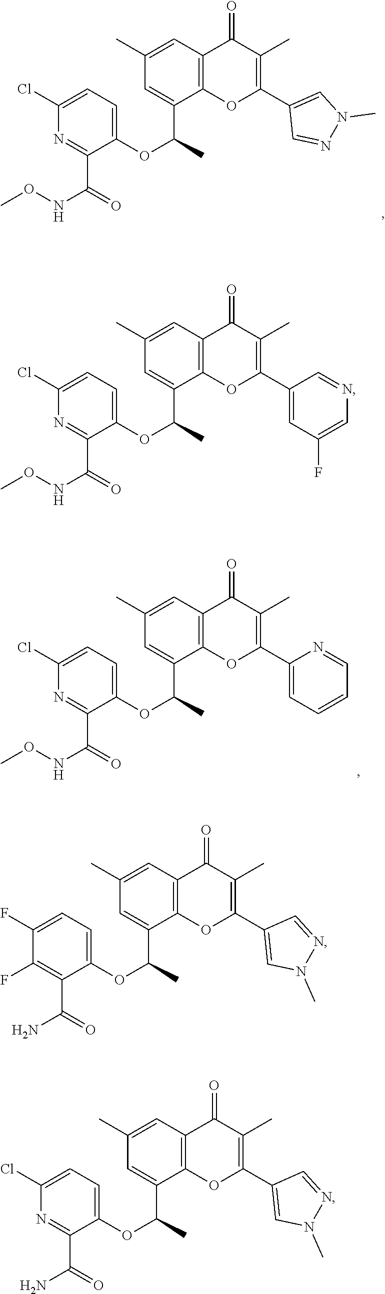

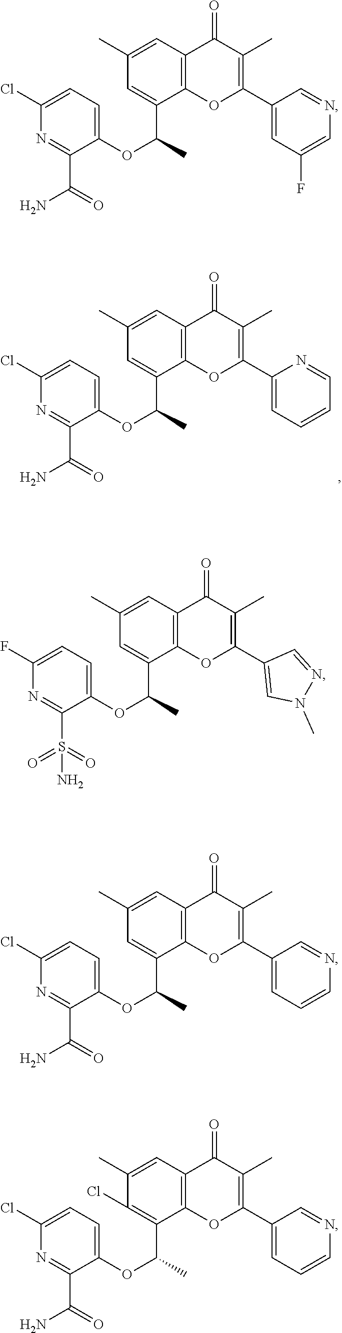 [SMILES: Cc1cc([C@@H](C)Oc2ccc(Cl)nc2C(N)=O)c2oc(-c3cncc(F)c3)c(C)c(=O)c2c1 ; Cc1cc([C@@H](C)Oc2ccc(Cl)nc2C(N)=O)c2oc(-c3ccccn3)c(C)c(=O)c2c1 ; Cc1cc([C@@H](C)Oc2ccc(F)nc2S(N)(=O)=O)c2oc(-c3cnn(C)c3)c(C)c(=O)c2c1 ; Cc1cc([C@@H](C)Oc2ccc(Cl)nc2C(N)=O)c2oc(-c3cccnc3)c(C)c(=O)c2c1 ; Cc1cc2c(=O)c(C)c(-c3cccnc3)oc2c([C@H](C)Oc2ccc(Cl)nc2C(N)=O)c1Cl]

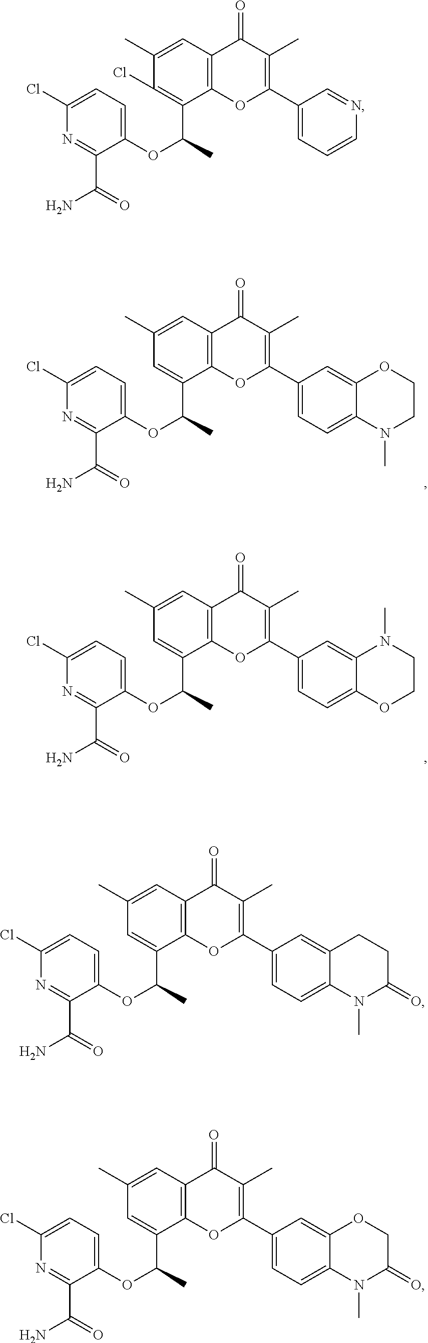 [SMILES: Cc1cc2c(=O)c(C)c(-c3cccnc3)oc2c([C@@H](C)Oc2ccc(Cl)nc2C(N)=O)c1Cl ; Cc1cc([C@@H](C)Oc2ccc(Cl)nc2C(N)=O)c2oc(-c3ccc4c(c3)OCCN4C)c(C)c(=O)c2c1 ; Cc1cc([C@@H](C)Oc2ccc(Cl)nc2C(N)=O)c2oc(-c3ccc4c(c3)N(C)CCO4)c(C)c(=O)c2c1 ; Cc1cc([C@@H](C)Oc2ccc(Cl)nc2C(N)=O)c2oc(-c3ccc4c(c3)CCC(=O)N4C)c(C)c(=O)c2c1 ; Cc1cc([C@@H](C)Oc2ccc(Cl)nc2C(N)=O)c2oc(-c3ccc4c(c3)OCC(=O)N4C)c(C)c(=O)c2c1]

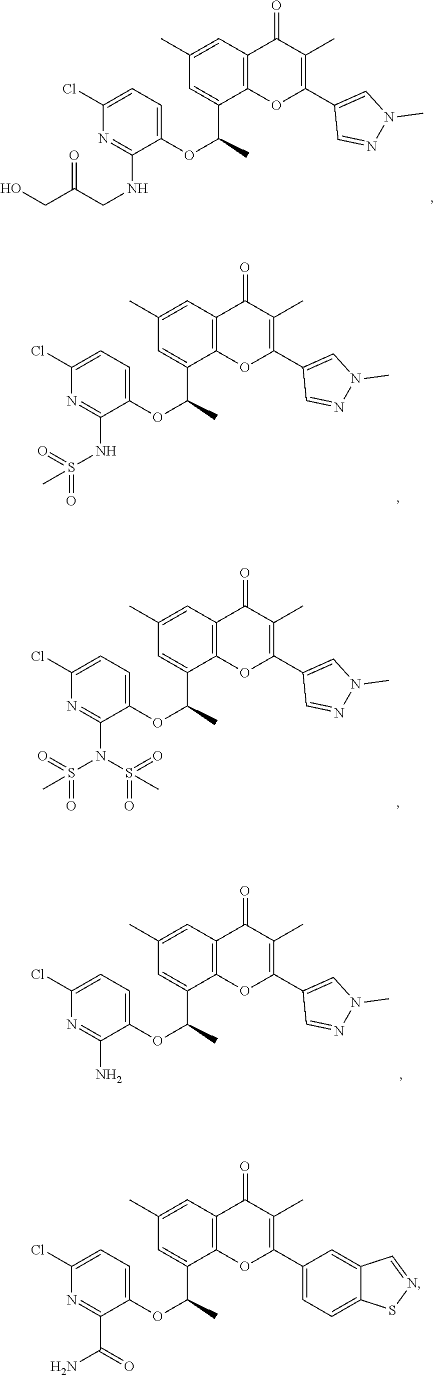 [SMILES: Cc1cc([C@@H](C)Oc2ccc(Cl)nc2NCC(=O)CO)c2oc(-c3cnn(C)c3)c(C)c(=O)c2c1 ; Cc1cc([C@@H](C)Oc2ccc(Cl)nc2NS(C)(=O)=O)c2oc(-c3cnn(C)c3)c(C)c(=O)c2c1 ; Cc1cc([C@@H](C)Oc2ccc(Cl)nc2N(S(C)(=O)=O)S(C)(=O)=O)c2oc(-c3cnn(C)c3)c(C)c(=O)c2c1 ; Cc1cc([C@@H](C)Oc2ccc(Cl)nc2N)c2oc(-c3cnn(C)c3)c(C)c(=O)c2c1 ; Cc1cc([C@@H](C)Oc2ccc(Cl)nc2C(N)=O)c2oc(-c3ccc4sncc4c3)c(C)c(=O)c2c1]

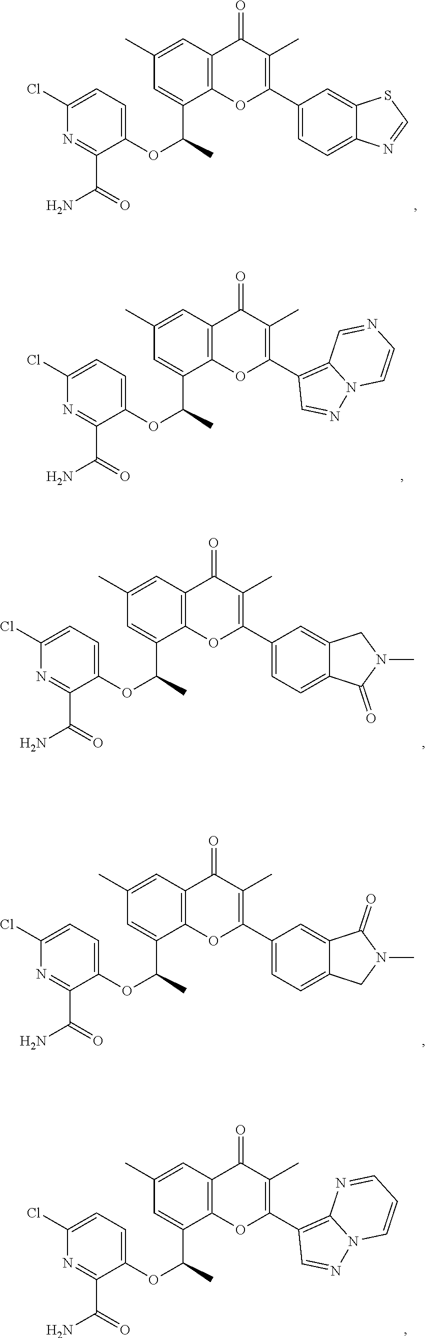 [SMILES: Cc1cc([C@@H](C)Oc2ccc(Cl)nc2C(N)=O)c2oc(-c3ccc4ncsc4c3)c(C)c(=O)c2c1 ; Cc1cc([C@@H](C)Oc2ccc(Cl)nc2C(N)=O)c2oc(-c3cnn4ccncc34)c(C)c(=O)c2c1 ; Cc1cc([C@@H](C)Oc2ccc(Cl)nc2C(N)=O)c2oc(-c3ccc4c(c3)CN(C)C4=O)c(C)c(=O)c2c1 ; Cc1cc([C@@H](C)Oc2ccc(Cl)nc2C(N)=O)c2oc(-c3ccc4c(c3)C(=O)N(C)C4)c(C)c(=O)c2c1 ; Cc1cc([C@@H](C)Oc2ccc(Cl)nc2C(N)=O)c2oc(-c3cnn4cccnc34)c(C)c(=O)c2c1]

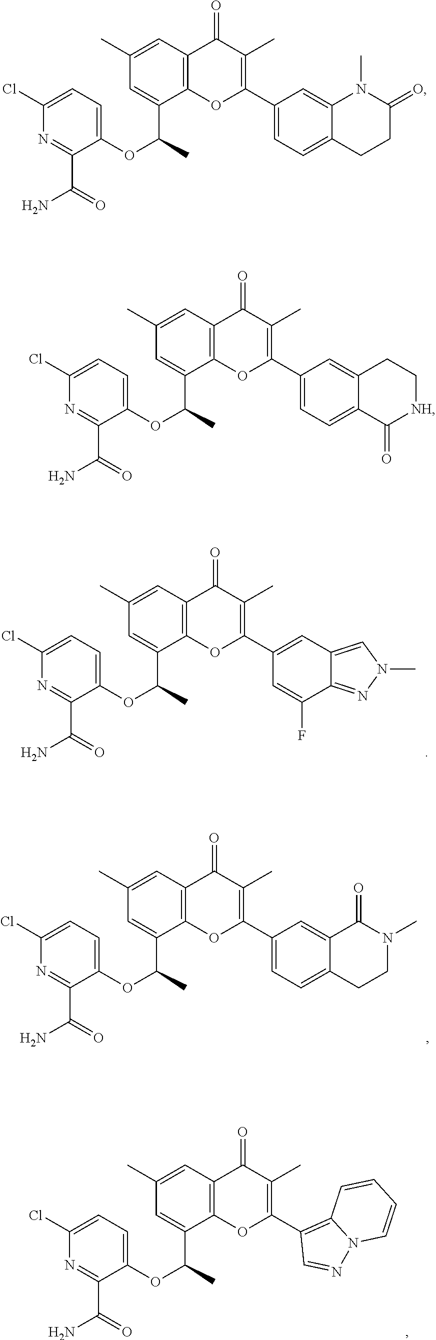 [SMILES: Cc1cc([C@@H](C)Oc2ccc(Cl)nc2C(N)=O)c2oc(-c3ccc4c(c3)N(C)C(=O)CC4)c(C)c(=O)c2c1 ; Cc1cc([C@@H](C)Oc2ccc(Cl)nc2C(N)=O)c2oc(-c3ccc4c(c3)CCNC4=O)c(C)c(=O)c2c1 ; Cc1cc([C@@H](C)Oc2ccc(Cl)nc2C(N)=O)c2oc(-c3cc(F)c4nn(C)cc4c3)c(C)c(=O)c2c1 ; Cc1cc([C@@H](C)Oc2ccc(Cl)nc2C(N)=O)c2oc(-c3ccc4c(c3)C(=O)N(C)CC4)c(C)c(=O)c2c1 ; Cc1cc([C@@H](C)Oc2ccc(Cl)nc2C(N)=O)c2oc(-c3cnn4ccccc34)c(C)c(=O)c2c1]

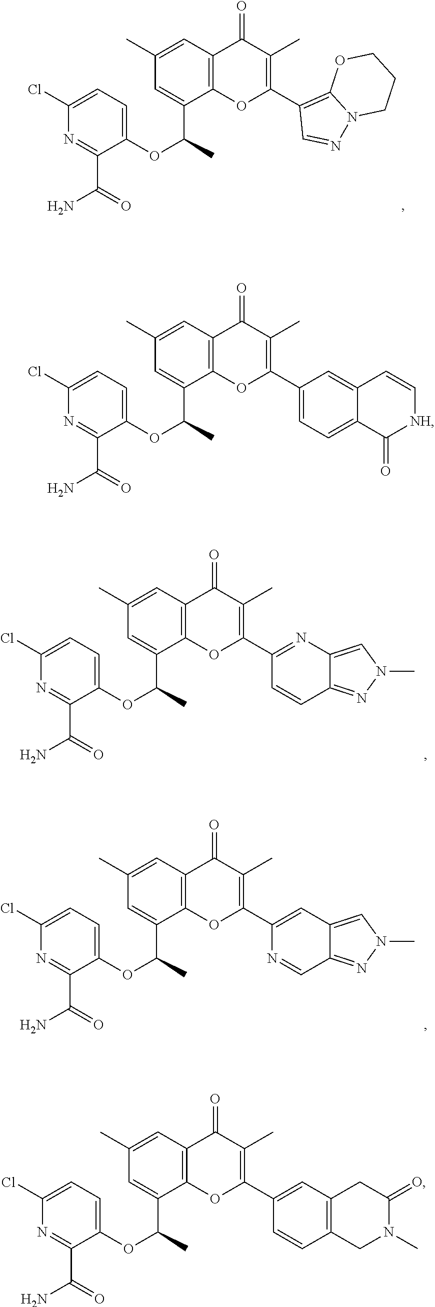 [SMILES: Cc1cc([C@@H](C)Oc2ccc(Cl)nc2C(N)=O)c2oc(-c3cnn4c3OCCC4)c(C)c(=O)c2c1 ; Cc1cc([C@@H](C)Oc2ccc(Cl)nc2C(N)=O)c2oc(-c3ccc4c(=O)[nH]ccc4c3)c(C)c(=O)c2c1 ; Cc1cc([C@@H](C)Oc2ccc(Cl)nc2C(N)=O)c2oc(-c3ccc4nn(C)cc4n3)c(C)c(=O)c2c1 ; Cc1cc([C@@H](C)Oc2ccc(Cl)nc2C(N)=O)c2oc(-c3cc4cn(C)nc4cn3)c(C)c(=O)c2c1 ; Cc1cc([C@@H](C)Oc2ccc(Cl)nc2C(N)=O)c2oc(-c3ccc4c(c3)CC(=O)N(C)C4)c(C)c(=O)c2c1]

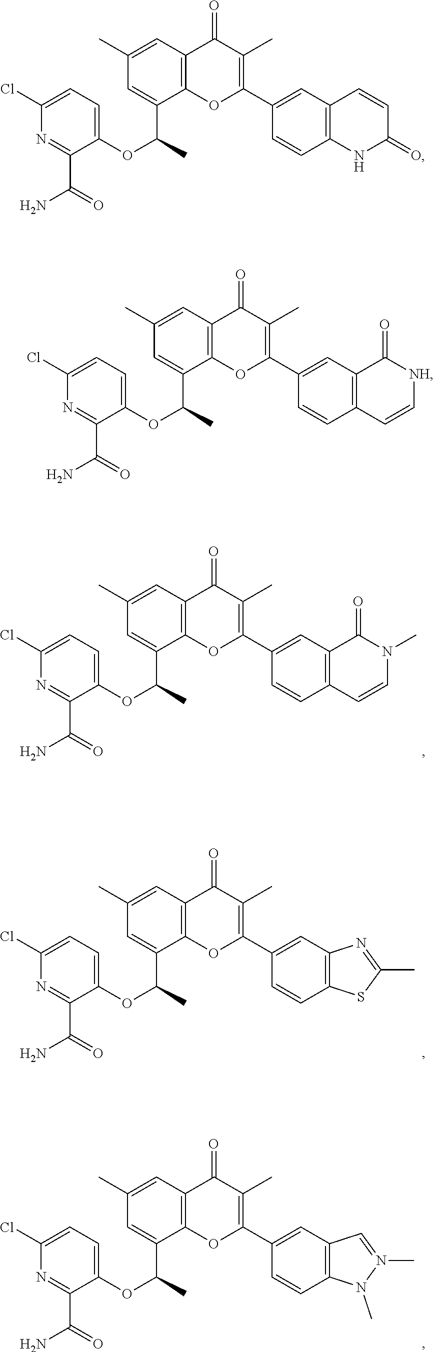

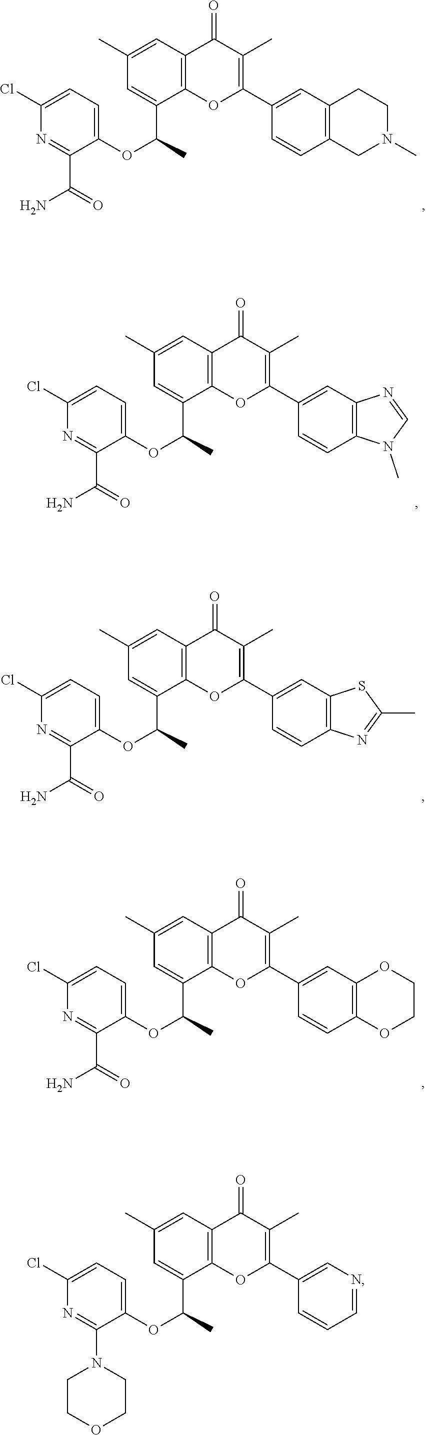 [SMILES: Cc1cc([C@@H](C)Oc2ccc(Cl)nc2C(N)=O)c2oc(-c3ccc4c(c3)CCN(C)C4)c(C)c(=O)c2c1 ; Cc1cc([C@@H](C)Oc2ccc(Cl)nc2C(N)=O)c2oc(-c3ccc4c(c3)ncn4C)c(C)c(=O)c2c1 ; Cc1cc([C@@H](C)Oc2ccc(Cl)nc2C(N)=O)c2oc(-c3ccc4nc(C)sc4c3)c(C)c(=O)c2c1 ; Cc1cc([C@@H](C)Oc2ccc(Cl)nc2C(N)=O)c2oc(-c3ccc4c(c3)OCCO4)c(C)c(=O)c2c1 ; Cc1cc([C@@H](C)Oc2ccc(Cl)nc2N2CCOCC2)c2oc(-c3cccnc3)c(C)c(=O)c2c1]

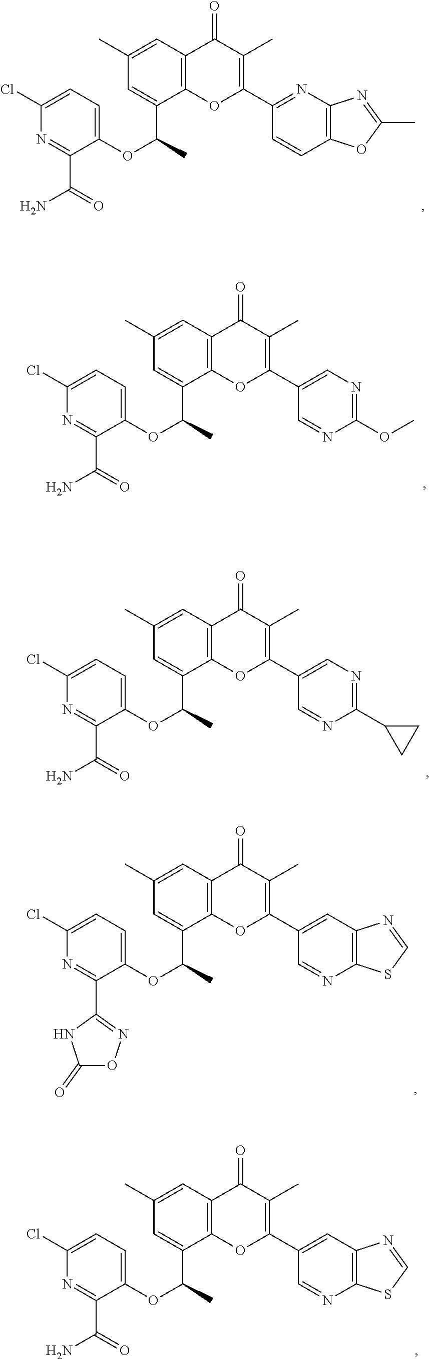 [SMILES: Cc1cc([C@@H](C)Oc2ccc(Cl)nc2C(N)=O)c2oc(-c3ccc4oc(C)nc4n3)c(C)c(=O)c2c1 ; COc1ncc(-c2oc3c([C@@H](C)Oc4ccc(Cl)nc4C(N)=O)cc(C)cc3c(=O)c2C)cn1 ; Cc1cc([C@@H](C)Oc2ccc(Cl)nc2C(N)=O)c2oc(-c3cnc(C4CC4)nc3)c(C)c(=O)c2c1 ; Cc1cc([C@@H](C)Oc2ccc(Cl)nc2-c2noc(=O)[nH]2)c2oc(-c3cnc4scnc4c3)c(C)c(=O)c2c1 ; Cc1cc([C@@H](C)Oc2ccc(Cl)nc2C(N)=O)c2oc(-c3cnc4scnc4c3)c(C)c(=O)c2c1]

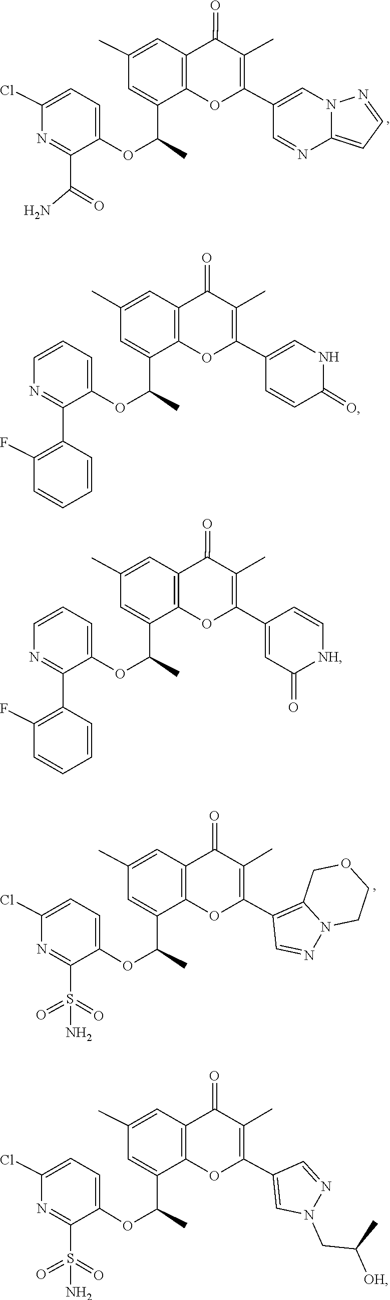 [SMILES: Cc1cc([C@@H](C)Oc2ccc(Cl)nc2C(N)=O)c2oc(-c3cnc4ccnn4c3)c(C)c(=O)c2c1 ; Cc1cc([C@@H](C)Oc2cccnc2-c2ccccc2F)c2oc(-c3ccc(=O)[nH]c3)c(C)c(=O)c2c1 ; Cc1cc([C@@H](C)Oc2cccnc2-c2ccccc2F)c2oc(-c3cc[nH]c(=O)c3)c(C)c(=O)c2c1 ; Cc1cc([C@@H](C)Oc2ccc(Cl)nc2S(N)(=O)=O)c2oc(-c3cnn4c3COCC4)c(C)c(=O)c2c1 ; Cc1cc([C@@H](C)Oc2ccc(Cl)nc2S(N)(=O)=O)c2oc(-c3cnn(C[C@@H](C)O)c3)c(C)c(=O)c2c1]

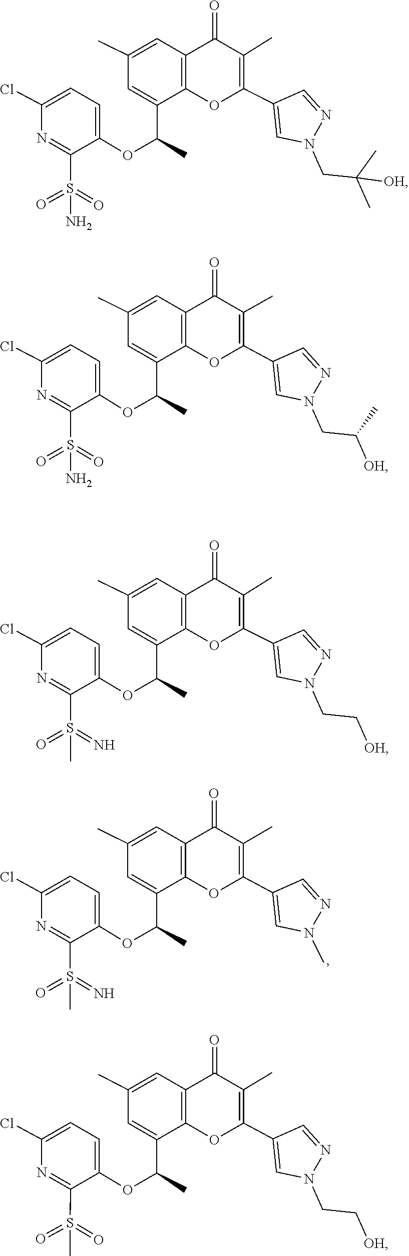 [SMILES: Cc1cc([C@@H](C)Oc2ccc(Cl)nc2S(N)(=O)=O)c2oc(-c3cnn(CC(C)(C)O)c3)c(C)c(=O)c2c1 ; Cc1cc([C@@H](C)Oc2ccc(Cl)nc2S(N)(=O)=O)c2oc(-c3cnn(C[C@H](C)O)c3)c(C)c(=O)c2c1 ; Cc1cc([C@@H](C)Oc2ccc(Cl)nc2S(C)(=N)=O)c2oc(-c3cnn(CCO)c3)c(C)c(=O)c2c1 ; Cc1cc([C@@H](C)Oc2ccc(Cl)nc2S(C)(=N)=O)c2oc(-c3cnn(C)c3)c(C)c(=O)c2c1 ; Cc1cc([C@@H](C)Oc2ccc(Cl)nc2S(C)(=O)=O)c2oc(-c3cnn(CCO)c3)c(C)c(=O)c2c1]

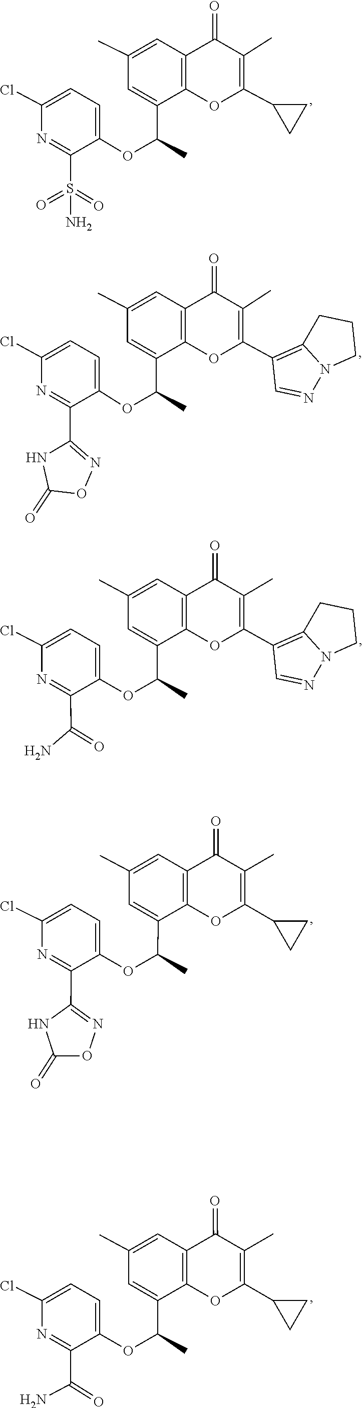 [SMILES: Cc1cc([C@@H](C)Oc2ccc(Cl)nc2S(N)(=O)=O)c2oc(C3CC3)c(C)c(=O)c2c1 ; Cc1cc([C@@H](C)Oc2ccc(Cl)nc2-c2noc(=O)[nH]2)c2oc(-c3cnn4c3CCC4)c(C)c(=O)c2c1 ; Cc1cc([C@@H](C)Oc2ccc(Cl)nc2C(N)=O)c2oc(-c3cnn4c3CCC4)c(C)c(=O)c2c1 ; Cc1cc([C@@H](C)Oc2ccc(Cl)nc2-c2noc(=O)[nH]2)c2oc(C3CC3)c(C)c(=O)c2c1 ; Cc1cc([C@@H](C)Oc2ccc(Cl)nc2C(N)=O)c2oc(C3CC3)c(C)c(=O)c2c1]

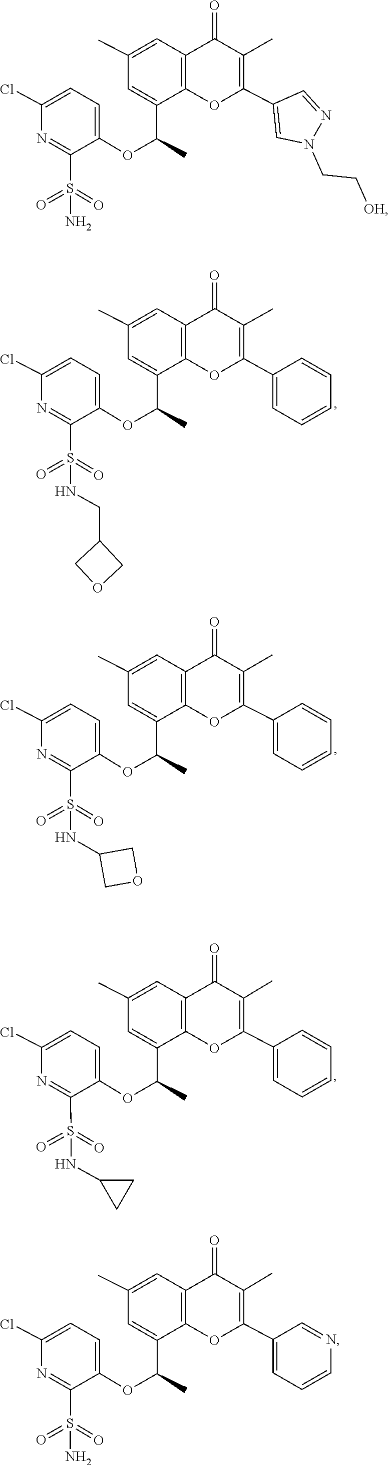 [SMILES: Cc1cc([C@@H](C)Oc2ccc(Cl)nc2S(N)(=O)=O)c2oc(-c3cnn(CCO)c3)c(C)c(=O)c2c1 ; Cc1cc([C@@H](C)Oc2ccc(Cl)nc2S(=O)(=O)NCC2COC2)c2oc(-c3ccccc3)c(C)c(=O)c2c1 ; Cc1cc([C@@H](C)Oc2ccc(Cl)nc2S(=O)(=O)NC2COC2)c2oc(-c3ccccc3)c(C)c(=O)c2c1 ; Cc1cc([C@@H](C)Oc2ccc(Cl)nc2S(=O)(=O)NC2CC2)c2oc(-c3ccccc3)c(C)c(=O)c2c1 ; Cc1cc([C@@H](C)Oc2ccc(Cl)nc2S(N)(=O)=O)c2oc(-c3cccnc3)c(C)c(=O)c2c1]

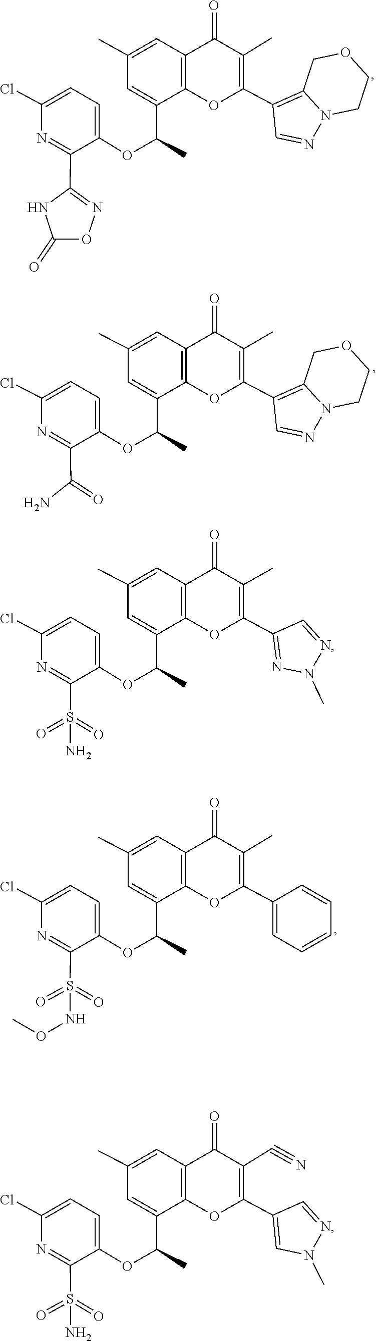 [SMILES: Cc1cc([C@@H](C)Oc2ccc(Cl)nc2-c2noc(=O)[nH]2)c2oc(-c3cnn4c3COCC4)c(C)c(=O)c2c1 ; Cc1cc([C@@H](C)Oc2ccc(Cl)nc2C(N)=O)c2oc(-c3cnn4c3COCC4)c(C)c(=O)c2c1 ; Cc1cc([C@@H](C)Oc2ccc(Cl)nc2S(N)(=O)=O)c2oc(-c3cnn(C)n3)c(C)c(=O)c2c1 ; CONS(=O)(=O)c1nc(Cl)ccc1O[C@H](C)c1cc(C)cc2c(=O)c(C)c(-c3ccccc3)oc12 ; Cc1cc([C@@H](C)Oc2ccc(Cl)nc2S(N)(=O)=O)c2oc(-c3cnn(C)c3)c(C#N)c(=O)c2c1]

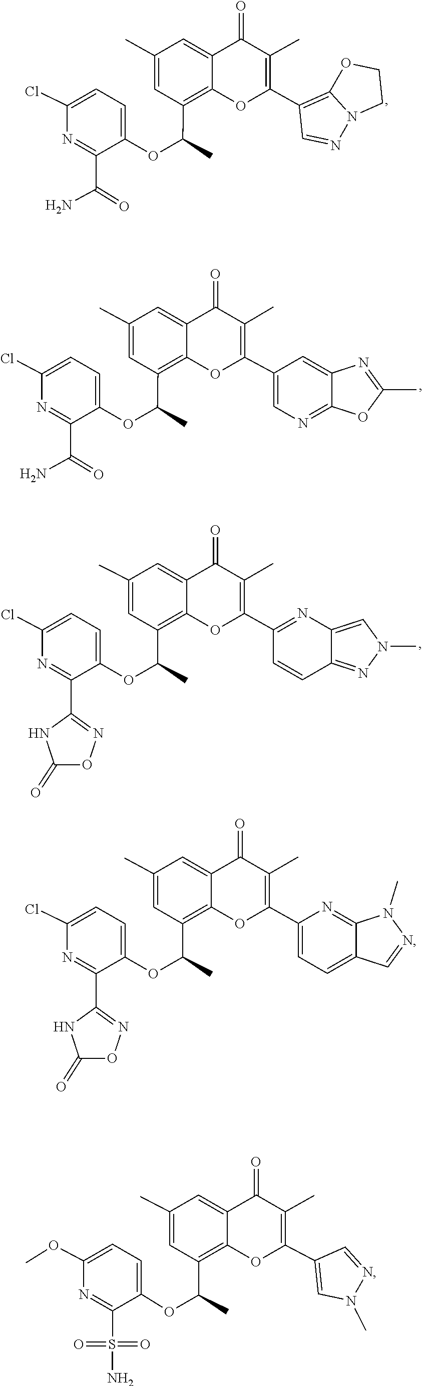

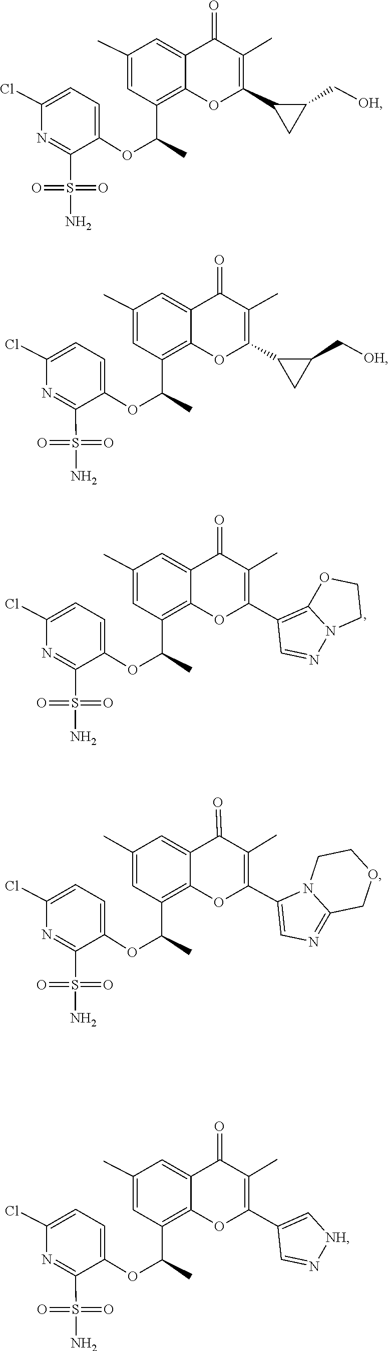

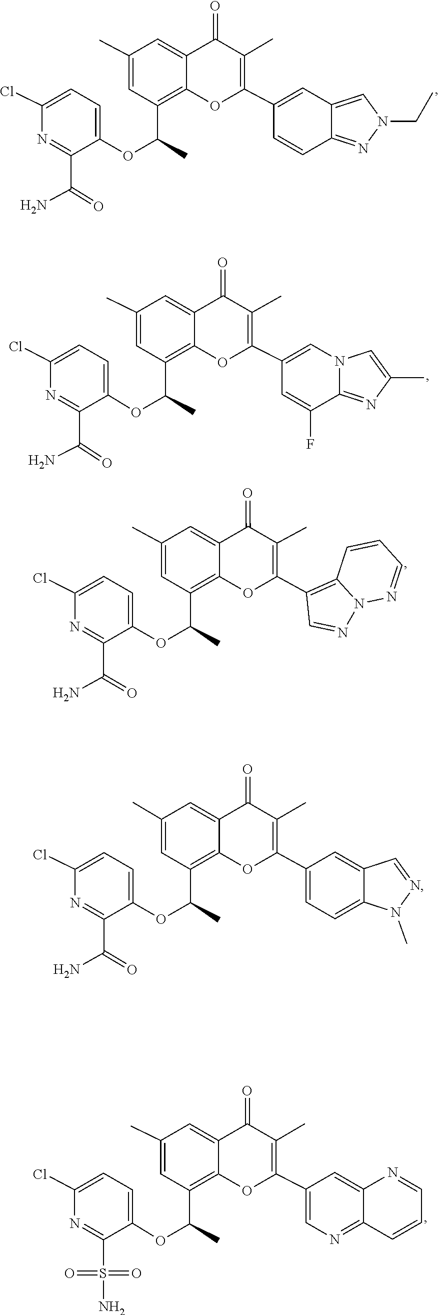 [SMILES: CCn1cc2cc(-c3oc4c([C@@H](C)Oc5ccc(Cl)nc5C(N)=O)cc(C)cc4c(=O)c3C)ccc2n1 ; Cc1cc([C@@H](C)Oc2ccc(Cl)nc2C(N)=O)c2oc(-c3cc(F)c4nc(C)cn4c3)c(C)c(=O)c2c1 ; Cc1cc([C@@H](C)Oc2ccc(Cl)nc2C(N)=O)c2oc(-c3cnn4ncccc34)c(C)c(=O)c2c1 ; Cc1cc([C@@H](C)Oc2ccc(Cl)nc2C(N)=O)c2oc(-c3ccc4c(cnn4C)c3)c(C)c(=O)c2c1 ; Cc1cc([C@@H](C)Oc2ccc(Cl)nc2S(N)(=O)=O)c2oc(-c3cnc4cccnc4c3)c(C)c(=O)c2c1]

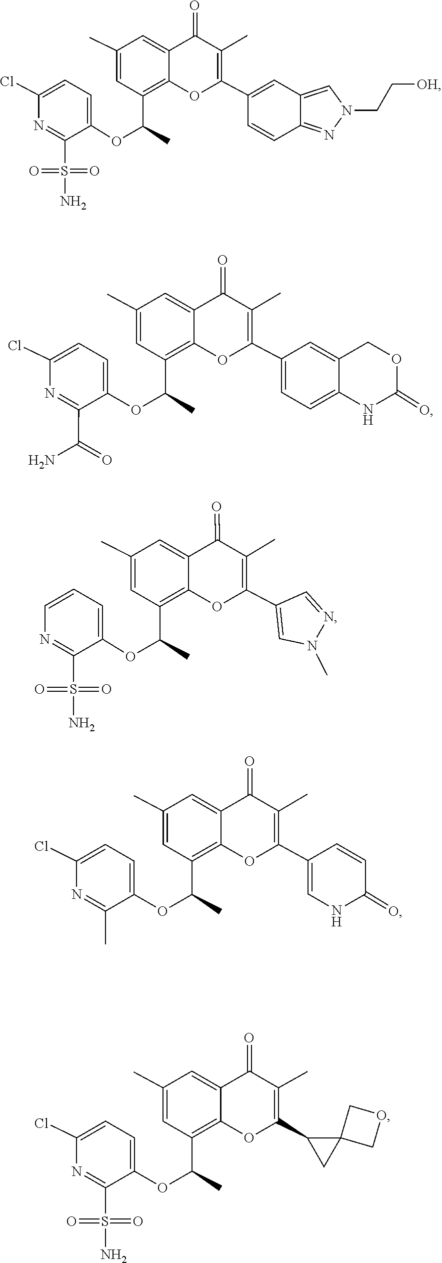 [SMILES: Cc1cc([C@@H](C)Oc2ccc(Cl)nc2S(N)(=O)=O)c2oc(-c3ccc4nn(CCO)cc4c3)c(C)c(=O)c2c1 ; Cc1cc([C@@H](C)Oc2ccc(Cl)nc2C(N)=O)c2oc(-c3ccc4c(c3)COC(=O)N4)c(C)c(=O)c2c1 ; Cc1cc([C@@H](C)Oc2cccnc2S(N)(=O)=O)c2oc(-c3cnn(C)c3)c(C)c(=O)c2c1 ; Cc1cc([C@@H](C)Oc2ccc(Cl)nc2C)c2oc(-c3ccc(=O)[nH]c3)c(C)c(=O)c2c1 ; Cc1cc([C@@H](C)Oc2ccc(Cl)nc2S(N)(=O)=O)c2oc([C@@H]3CC34COC4)c(C)c(=O)c2c1]

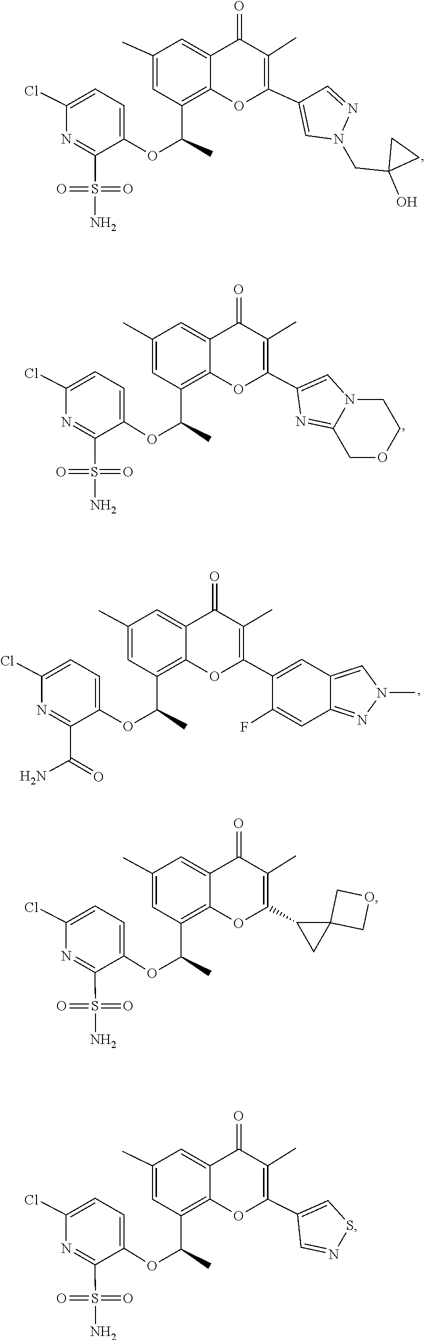 [SMILES: Cc1cc([C@@H](C)Oc2ccc(Cl)nc2S(N)(=O)=O)c2oc(-c3cnn(CC4(O)CC4)c3)c(C)c(=O)c2c1 ; Cc1cc([C@@H](C)Oc2ccc(Cl)nc2S(N)(=O)=O)c2oc(-c3cn4c(n3)COCC4)c(C)c(=O)c2c1 ; Cc1cc([C@@H](C)Oc2ccc(Cl)nc2C(N)=O)c2oc(-c3cc4cn(C)nc4cc3F)c(C)c(=O)c2c1 ; Cc1cc([C@@H](C)Oc2ccc(Cl)nc2S(N)(=O)=O)c2oc([C@H]3CC34COC4)c(C)c(=O)c2c1 ; Cc1cc([C@@H](C)Oc2ccc(Cl)nc2S(N)(=O)=O)c2oc(-c3cnsc3)c(C)c(=O)c2c1]

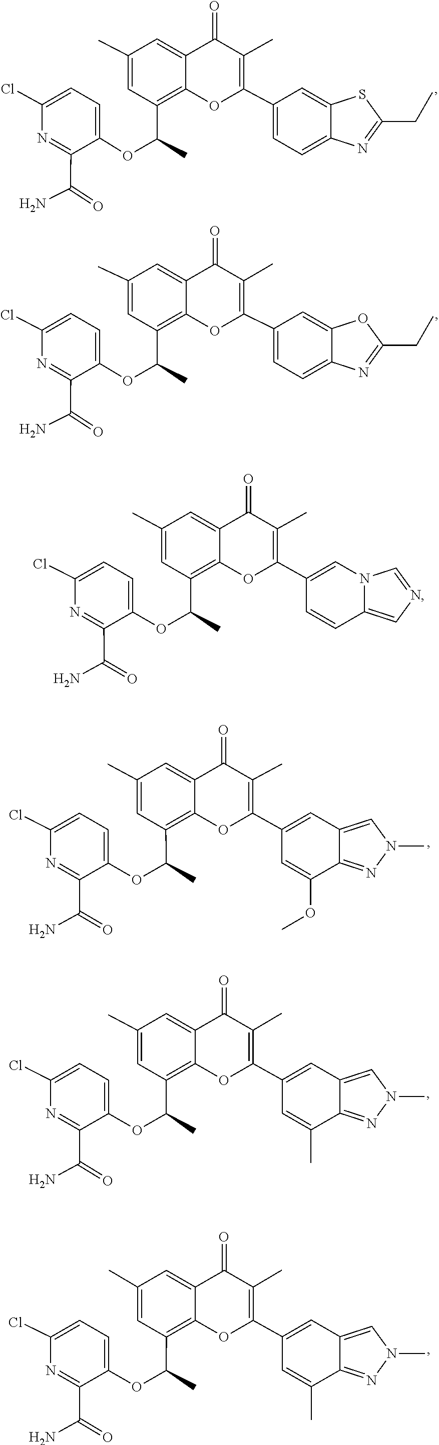 [SMILES: CCc1nc2ccc(-c3oc4c([C@@H](C)Oc5ccc(Cl)nc5C(N)=O)cc(C)cc4c(=O)c3C)cc2s1 ; CCc1nc2ccc(-c3oc4c([C@@H](C)Oc5ccc(Cl)nc5C(N)=O)cc(C)cc4c(=O)c3C)cc2o1 ; Cc1cc([C@@H](C)Oc2ccc(Cl)nc2C(N)=O)c2oc(-c3ccc4cncn4c3)c(C)c(=O)c2c1 ; COc1cc(-c2oc3c([C@@H](C)Oc4ccc(Cl)nc4C(N)=O)cc(C)cc3c(=O)c2C)cc2cn(C)nc12 ; Cc1cc([C@@H](C)Oc2ccc(Cl)nc2C(N)=O)c2oc(-c3cc(C)c4nn(C)cc4c3)c(C)c(=O)c2c1 ; Cc1cc([C@@H](C)Oc2ccc(Cl)nc2C(N)=O)c2oc(-c3cc(C)c4nn(C)cc4c3)c(C)c(=O)c2c1]

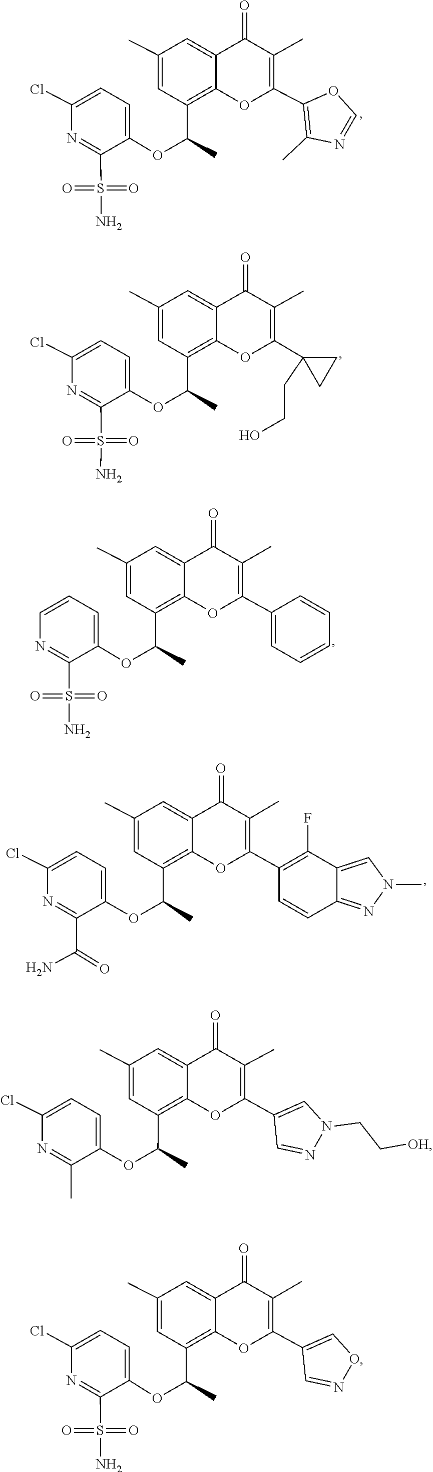 [SMILES: Cc1cc([C@@H](C)Oc2ccc(Cl)nc2S(N)(=O)=O)c2oc(-c3ocnc3C)c(C)c(=O)c2c1 ; Cc1cc([C@@H](C)Oc2ccc(Cl)nc2S(N)(=O)=O)c2oc(C3(CCO)CC3)c(C)c(=O)c2c1 ; Cc1cc([C@@H](C)Oc2cccnc2S(N)(=O)=O)c2oc(-c3ccccc3)c(C)c(=O)c2c1 ; Cc1cc([C@@H](C)Oc2ccc(Cl)nc2C(N)=O)c2oc(-c3ccc4nn(C)cc4c3F)c(C)c(=O)c2c1 ; Cc1cc([C@@H](C)Oc2ccc(Cl)nc2C)c2oc(-c3cnn(CCO)c3)c(C)c(=O)c2c1 ; Cc1cc([C@@H](C)Oc2ccc(Cl)nc2S(N)(=O)=O)c2oc(-c3cnoc3)c(C)c(=O)c2c1]

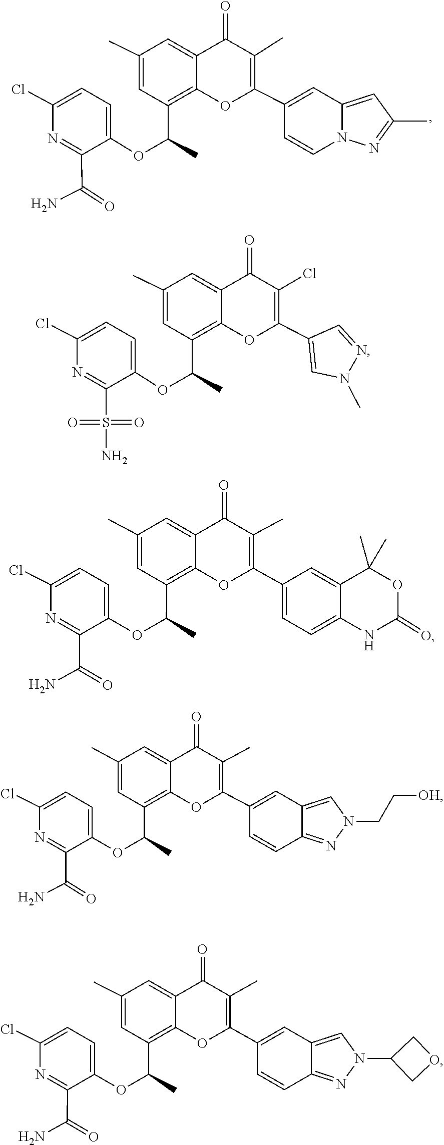 [SMILES: Cc1cc([C@@H](C)Oc2ccc(Cl)nc2C(N)=O)c2oc(-c3ccn4nc(C)cc4c3)c(C)c(=O)c2c1 ; Cc1cc([C@@H](C)Oc2ccc(Cl)nc2S(N)(=O)=O)c2oc(-c3cnn(C)c3)c(Cl)c(=O)c2c1 ; Cc1cc([C@@H](C)Oc2ccc(Cl)nc2C(N)=O)c2oc(-c3ccc4c(c3)C(C)(C)OC(=O)N4)c(C)c(=O)c2c1 ; Cc1cc([C@@H](C)Oc2ccc(Cl)nc2C(N)=O)c2oc(-c3ccc4nn(CCO)cc4c3)c(C)c(=O)c2c1 ; Cc1cc([C@@H](C)Oc2ccc(Cl)nc2C(N)=O)c2oc(-c3ccc4nn(C5COC5)cc4c3)c(C)c(=O)c2c1]

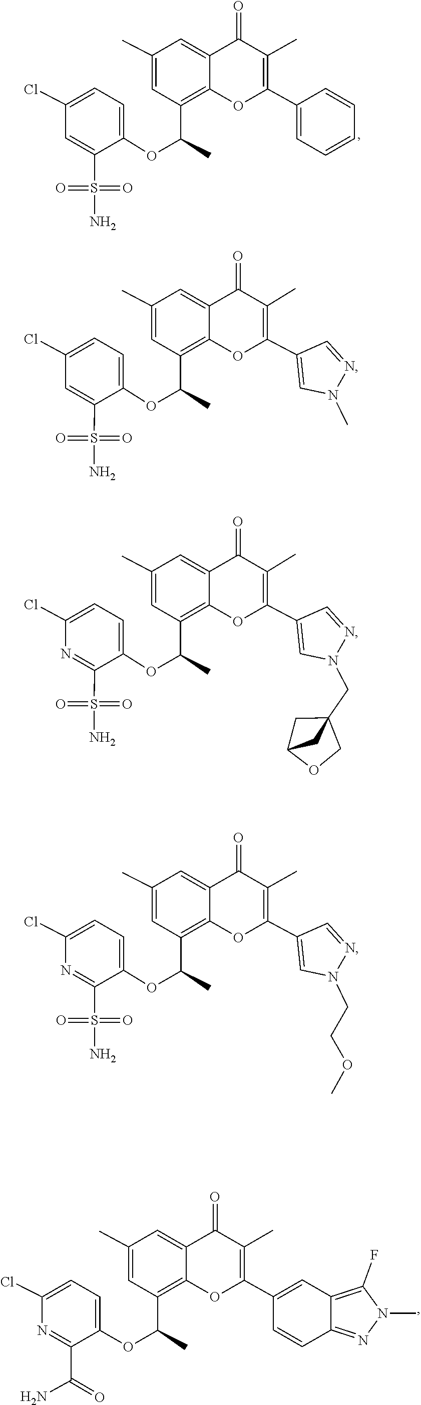 [SMILES: Cc1cc([C@@H](C)Oc2ccc(Cl)cc2S(N)(=O)=O)c2oc(-c3ccccc3)c(C)c(=O)c2c1 ; Cc1cc([C@@H](C)Oc2ccc(Cl)cc2S(N)(=O)=O)c2oc(-c3cnn(C)c3)c(C)c(=O)c2c1 ; Cc1cc([C@@H](C)Oc2ccc(Cl)nc2S(N)(=O)=O)c2oc(-c3cnn(C[C@]45CO[C@H](C4)C5)c3)c(C)c(=O)c2c1 ; COCCn1cc(-c2oc3c([C@@H](C)Oc4ccc(Cl)nc4S(N)(=O)=O)cc(C)cc3c(=O)c2C)cn1 ; Cc1cc([C@@H](C)Oc2ccc(Cl)nc2C(N)=O)c2oc(-c3ccc4nn(C)c(F)c4c3)c(C)c(=O)c2c1]

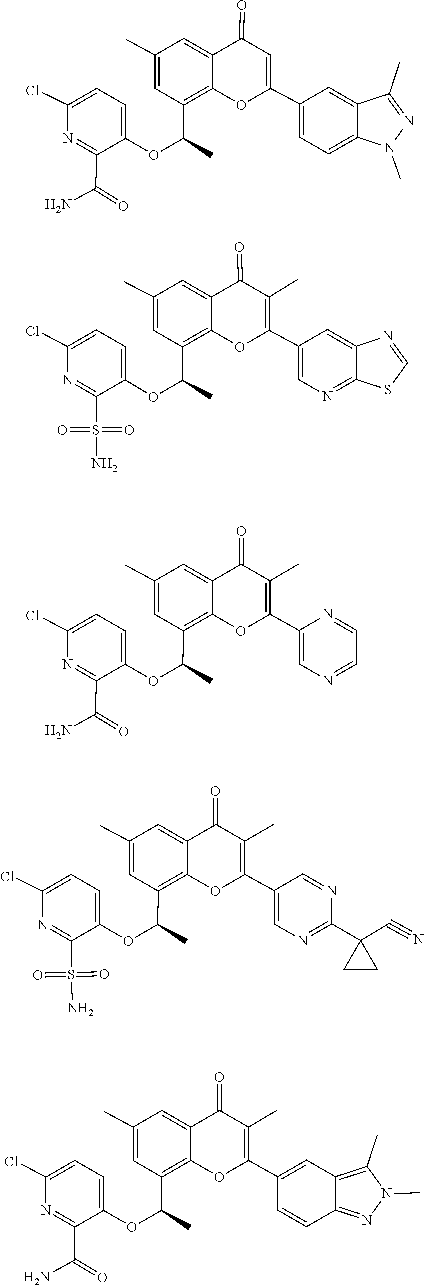 [SMILES: Cc1cc([C@@H](C)Oc2ccc(Cl)nc2C(N)=O)c2oc(-c3ccc4c(c3)c(C)nn4C)cc(=O)c2c1 ; Cc1cc([C@@H](C)Oc2ccc(Cl)nc2S(N)(=O)=O)c2oc(-c3cnc4scnc4c3)c(C)c(=O)c2c1 ; Cc1cc([C@@H](C)Oc2ccc(Cl)nc2C(N)=O)c2oc(-c3cnccn3)c(C)c(=O)c2c1 ; Cc1cc([C@@H](C)Oc2ccc(Cl)nc2S(N)(=O)=O)c2oc(-c3cnc(C4(C#N)CC4)nc3)c(C)c(=O)c2c1 ; Cc1cc([C@@H](C)Oc2ccc(Cl)nc2C(N)=O)c2oc(-c3ccc4nn(C)c(C)c4c3)c(C)c(=O)c2c1]

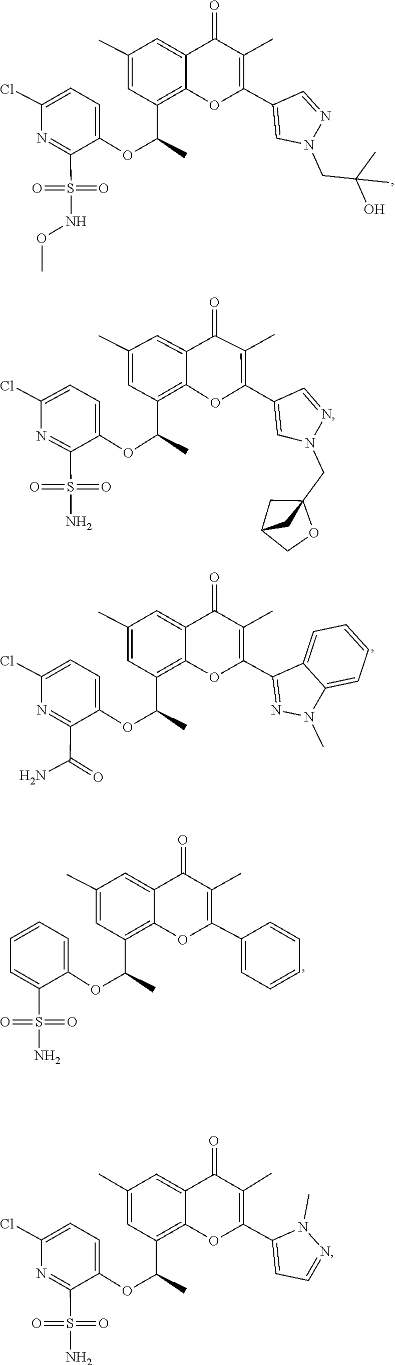 [SMILES: CONS(=O)(=O)c1nc(Cl)ccc1O[C@H](C)c1cc(C)cc2c(=O)c(C)c(-c3cnn(CC(C)(C)O)c3)oc12 ; Cc1cc([C@@H](C)Oc2ccc(Cl)nc2S(N)(=O)=O)c2oc(-c3cnn(C[C@]45C[C@H](CO4)C5)c3)c(C)c(=O)c2c1 ; Cc1cc([C@@H](C)Oc2ccc(Cl)nc2C(N)=O)c2oc(-c3nn(C)c4ccccc34)c(C)c(=O)c2c1 ; Cc1cc([C@@H](C)Oc2ccccc2S(N)(=O)=O)c2oc(-c3ccccc3)c(C)c(=O)c2c1 ; Cc1cc([C@@H](C)Oc2ccc(Cl)nc2S(N)(=O)=O)c2oc(-c3ccnn3C)c(C)c(=O)c2c1]

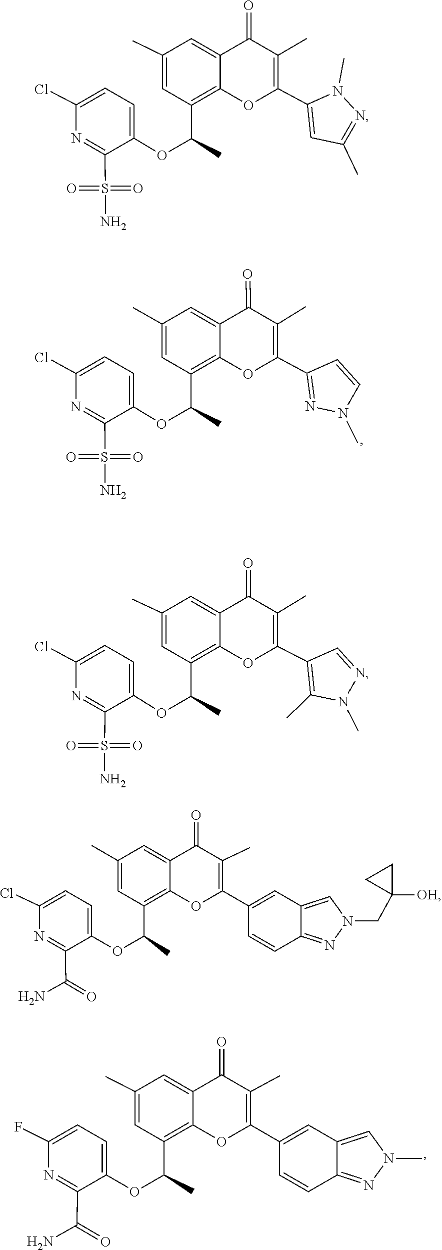 [SMILES: Cc1cc([C@@H](C)Oc2ccc(Cl)nc2S(N)(=O)=O)c2oc(-c3cc(C)nn3C)c(C)c(=O)c2c1 ; Cc1cc([C@@H](C)Oc2ccc(Cl)nc2S(N)(=O)=O)c2oc(-c3ccn(C)n3)c(C)c(=O)c2c1 ; Cc1cc([C@@H](C)Oc2ccc(Cl)nc2S(N)(=O)=O)c2oc(-c3cnn(C)c3C)c(C)c(=O)c2c1 ; Cc1cc([C@@H](C)Oc2ccc(Cl)nc2C(N)=O)c2oc(-c3ccc4nn(CC5(O)CC5)cc4c3)c(C)c(=O)c2c1 ; Cc1cc([C@@H](C)Oc2ccc(F)nc2C(N)=O)c2oc(-c3ccc4nn(C)cc4c3)c(C)c(=O)c2c1]

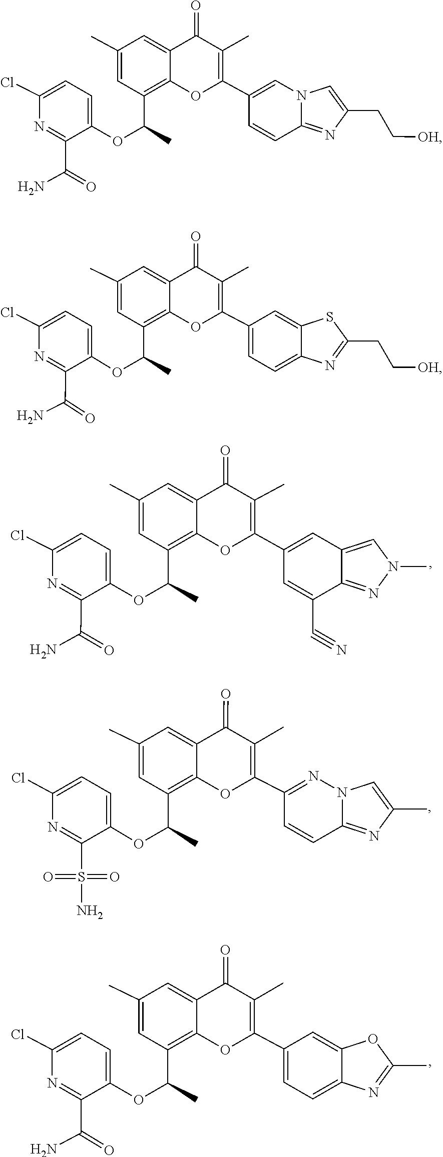 [SMILES: Cc1cc([C@@H](C)Oc2ccc(Cl)nc2C(N)=O)c2oc(-c3ccc4nc(CCO)cn4c3)c(C)c(=O)c2c1 ; Cc1cc([C@@H](C)Oc2ccc(Cl)nc2C(N)=O)c2oc(-c3ccc4nc(CCO)sc4c3)c(C)c(=O)c2c1 ; Cc1cc([C@@H](C)Oc2ccc(Cl)nc2C(N)=O)c2oc(-c3cc(C#N)c4nn(C)cc4c3)c(C)c(=O)c2c1 ; Cc1cc([C@@H](C)Oc2ccc(Cl)nc2S(N)(=O)=O)c2oc(-c3ccc4nc(C)cn4n3)c(C)c(=O)c2c1 ; Cc1cc([C@@H](C)Oc2ccc(Cl)nc2C(N)=O)c2oc(-c3ccc4nc(C)oc4c3)c(C)c(=O)c2c1]

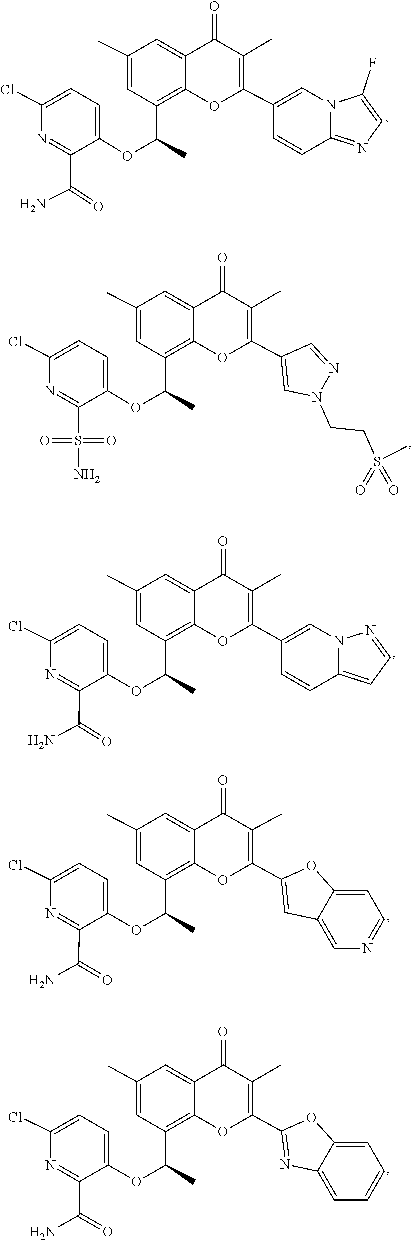 [SMILES: Cc1cc([C@@H](C)Oc2ccc(Cl)nc2C(N)=O)c2oc(-c3ccc4ncc(F)n4c3)c(C)c(=O)c2c1 ; Cc1cc([C@@H](C)Oc2ccc(Cl)nc2S(N)(=O)=O)c2oc(-c3cnn(CCS(C)(=O)=O)c3)c(C)c(=O)c2c1 ; Cc1cc([C@@H](C)Oc2ccc(Cl)nc2C(N)=O)c2oc(-c3ccc4ccnn4c3)c(C)c(=O)c2c1 ; Cc1cc([C@@H](C)Oc2ccc(Cl)nc2C(N)=O)c2oc(-c3cc4cnccc4o3)c(C)c(=O)c2c1 ; Cc1cc([C@@H](C)Oc2ccc(Cl)nc2C(N)=O)c2oc(-c3nc4ccccc4o3)c(C)c(=O)c2c1]

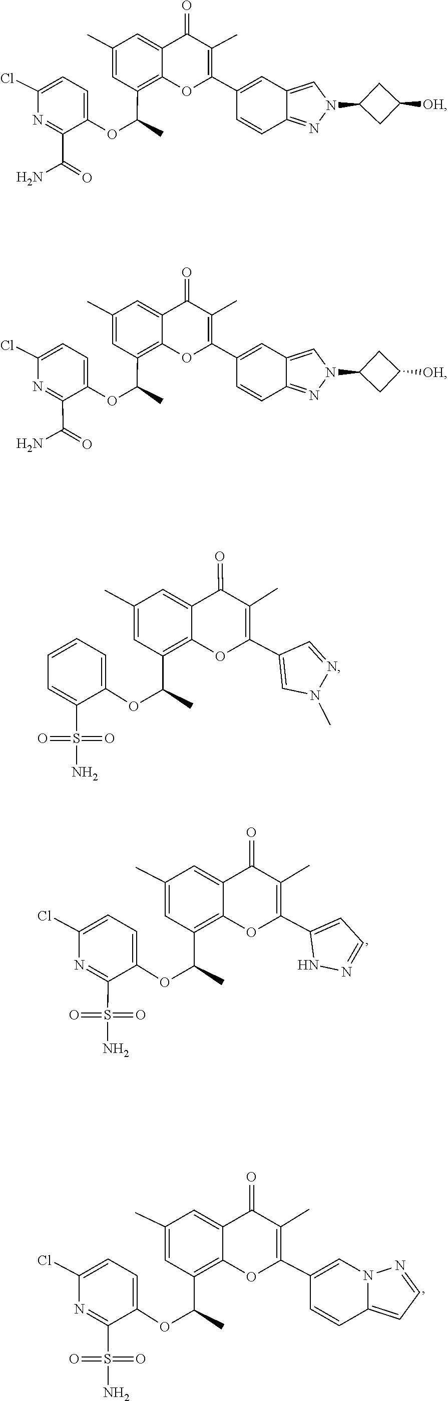 [SMILES: Cc1cc([C@@H](C)Oc2ccc(Cl)nc2C(N)=O)c2oc(-c3ccc4nn([C@H]5C[C@@H](O)C5)cc4c3)c(C)c(=O)c2c1 ; Cc1cc([C@@H](C)Oc2ccc(Cl)nc2C(N)=O)c2oc(-c3ccc4nn([C@H]5C[C@H](O)C5)cc4c3)c(C)c(=O)c2c1 ; Cc1cc([C@@H](C)Oc2ccccc2S(N)(=O)=O)c2oc(-c3cnn(C)c3)c(C)c(=O)c2c1 ; Cc1cc([C@@H](C)Oc2ccc(Cl)nc2S(N)(=O)=O)c2oc(-c3ccn[nH]3)c(C)c(=O)c2c1 ; Cc1cc([C@@H](C)Oc2ccc(Cl)nc2S(N)(=O)=O)c2oc(-c3ccc4ccnn4c3)c(C)c(=O)c2c1]

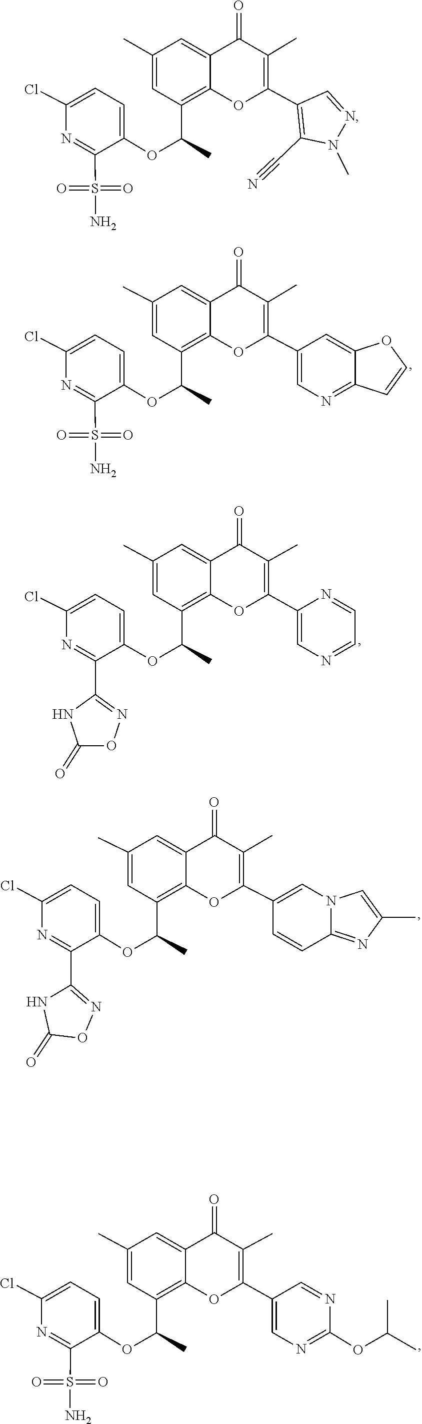 [SMILES: Cc1cc([C@@H](C)Oc2ccc(Cl)nc2S(N)(=O)=O)c2oc(-c3cnn(C)c3C#N)c(C)c(=O)c2c1 ; Cc1cc([C@@H](C)Oc2ccc(Cl)nc2S(N)(=O)=O)c2oc(-c3cnc4ccoc4c3)c(C)c(=O)c2c1 ; Cc1cc([C@@H](C)Oc2ccc(Cl)nc2-c2noc(=O)[nH]2)c2oc(-c3cnccn3)c(C)c(=O)c2c1 ; Cc1cc([C@@H](C)Oc2ccc(Cl)nc2-c2noc(=O)[nH]2)c2oc(-c3ccc4nc(C)cn4c3)c(C)c(=O)c2c1 ; Cc1cc([C@@H](C)Oc2ccc(Cl)nc2S(N)(=O)=O)c2oc(-c3cnc(OC(C)C)nc3)c(C)c(=O)c2c1]

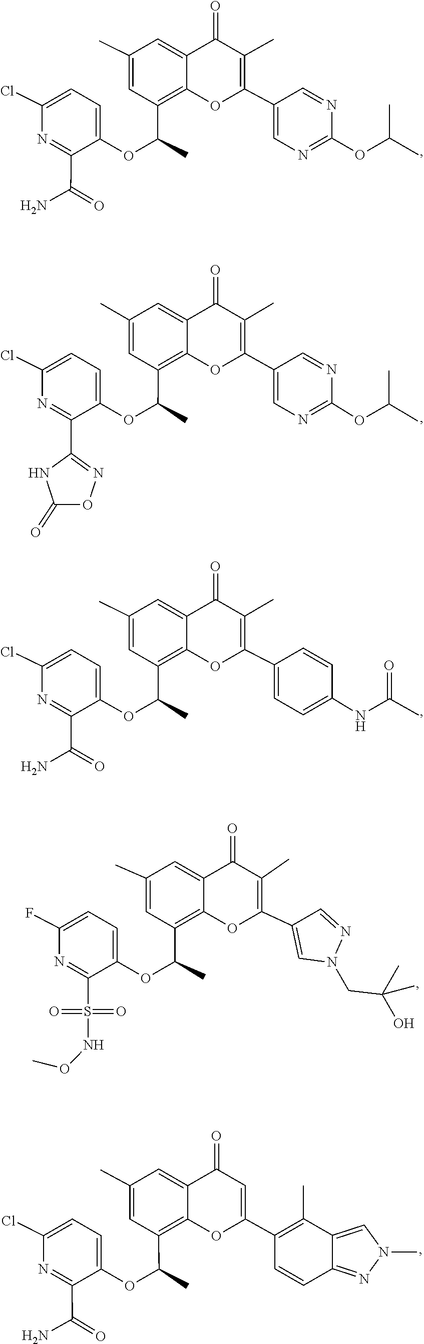 [SMILES: Cc1cc([C@@H](C)Oc2ccc(Cl)nc2C(N)=O)c2oc(-c3cnc(OC(C)C)nc3)c(C)c(=O)c2c1 ; Cc1cc([C@@H](C)Oc2ccc(Cl)nc2-c2noc(=O)[nH]2)c2oc(-c3cnc(OC(C)C)nc3)c(C)c(=O)c2c1 ; CC(=O)Nc1ccc(-c2oc3c([C@@H](C)Oc4ccc(Cl)nc4C(N)=O)cc(C)cc3c(=O)c2C)cc1 ; CONS(=O)(=O)c1nc(F)ccc1O[C@H](C)c1cc(C)cc2c(=O)c(C)c(-c3cnn(CC(C)(C)O)c3)oc12 ; Cc1cc([C@@H](C)Oc2ccc(Cl)nc2C(N)=O)c2oc(-c3ccc4nn(C)cc4c3C)cc(=O)c2c1]

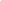

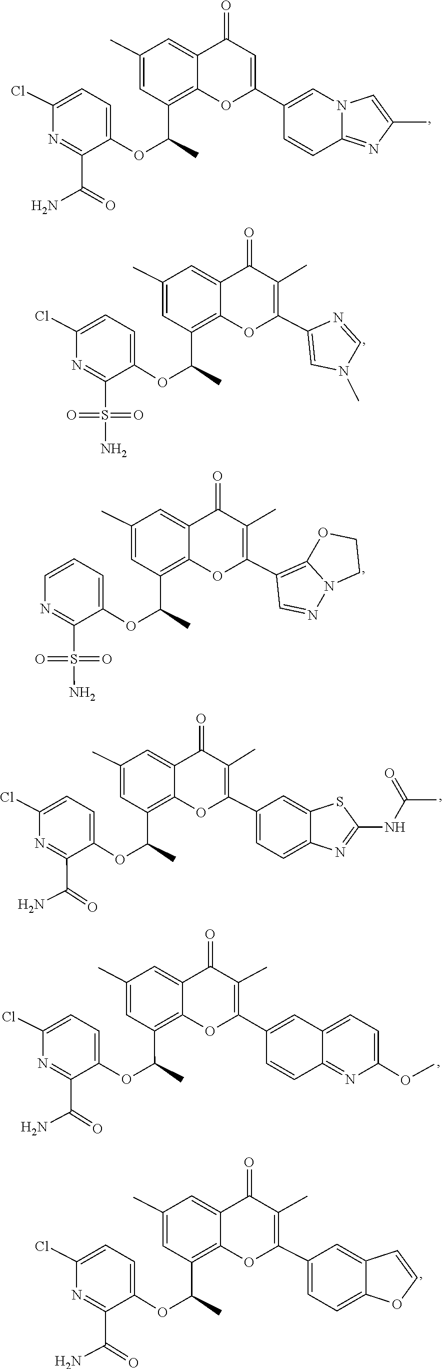 [SMILES: Cc1cc([C@@H](C)Oc2ccc(Cl)nc2C(N)=O)c2oc(-c3ccc4nc(C)cn4c3)cc(=O)c2c1 ; Cc1cc([C@@H](C)Oc2ccc(Cl)nc2S(N)(=O)=O)c2oc(-c3cn(C)cn3)c(C)c(=O)c2c1 ; Cc1cc([C@@H](C)Oc2cccnc2S(N)(=O)=O)c2oc(-c3cnn4c3OCC4)c(C)c(=O)c2c1 ; CC(=O)Nc1nc2ccc(-c3oc4c([C@@H](C)Oc5ccc(Cl)nc5C(N)=O)cc(C)cc4c(=O)c3C)cc2s1 ; COc1ccc2cc(-c3oc4c([C@@H](C)Oc5ccc(Cl)nc5C(N)=O)cc(C)cc4c(=O)c3C)ccc2n1 ; Cc1cc([C@@H](C)Oc2ccc(Cl)nc2C(N)=O)c2oc(-c3ccc4occc4c3)c(C)c(=O)c2c1]

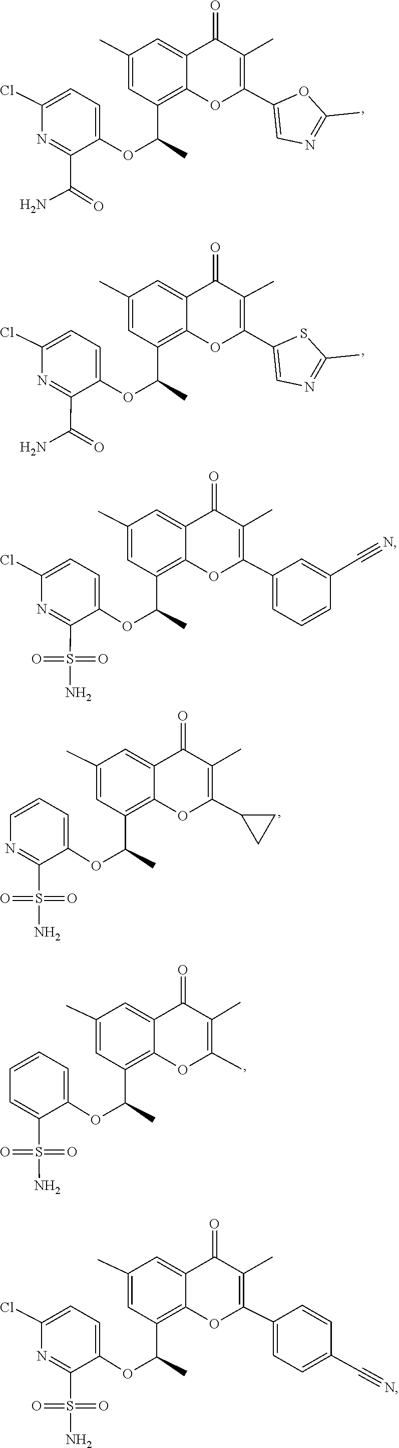 [SMILES: Cc1cc([C@@H](C)Oc2ccc(Cl)nc2C(N)=O)c2oc(-c3cnc(C)o3)c(C)c(=O)c2c1 ; Cc1cc([C@@H](C)Oc2ccc(Cl)nc2C(N)=O)c2oc(-c3cnc(C)s3)c(C)c(=O)c2c1 ; Cc1cc([C@@H](C)Oc2ccc(Cl)nc2S(N)(=O)=O)c2oc(-c3cccc(C#N)c3)c(C)c(=O)c2c1 ; Cc1cc([C@@H](C)Oc2cccnc2S(N)(=O)=O)c2oc(C3CC3)c(C)c(=O)c2c1 ; Cc1cc([C@@H](C)Oc2ccccc2S(N)(=O)=O)c2oc(C)c(C)c(=O)c2c1 ; Cc1cc([C@@H](C)Oc2ccc(Cl)nc2S(N)(=O)=O)c2oc(-c3ccc(C#N)cc3)c(C)c(=O)c2c1]

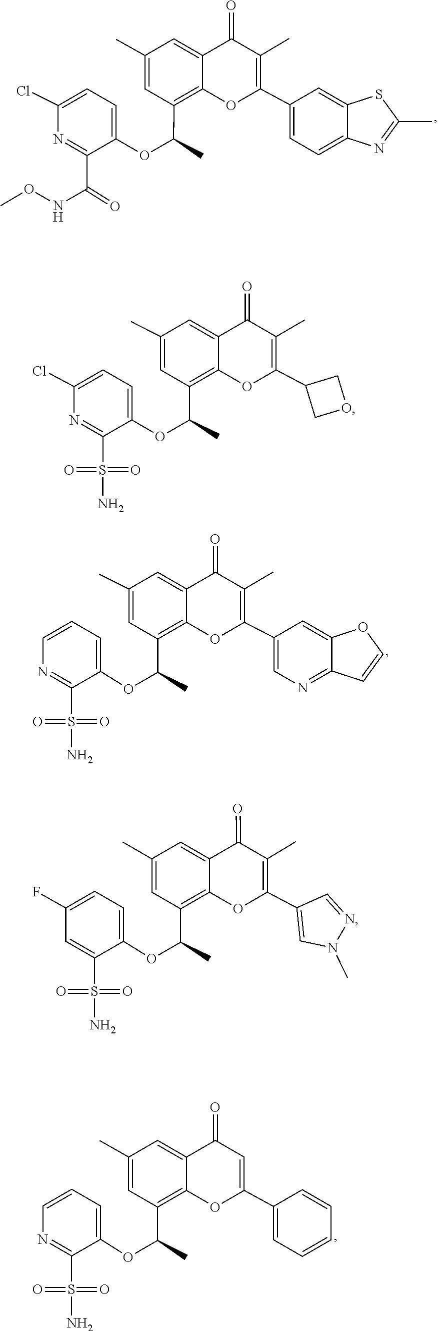

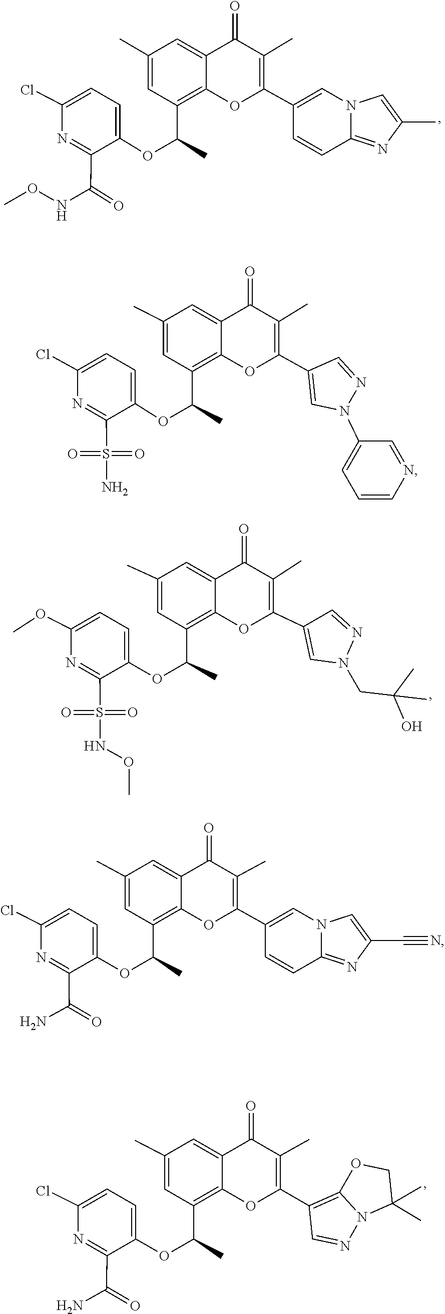

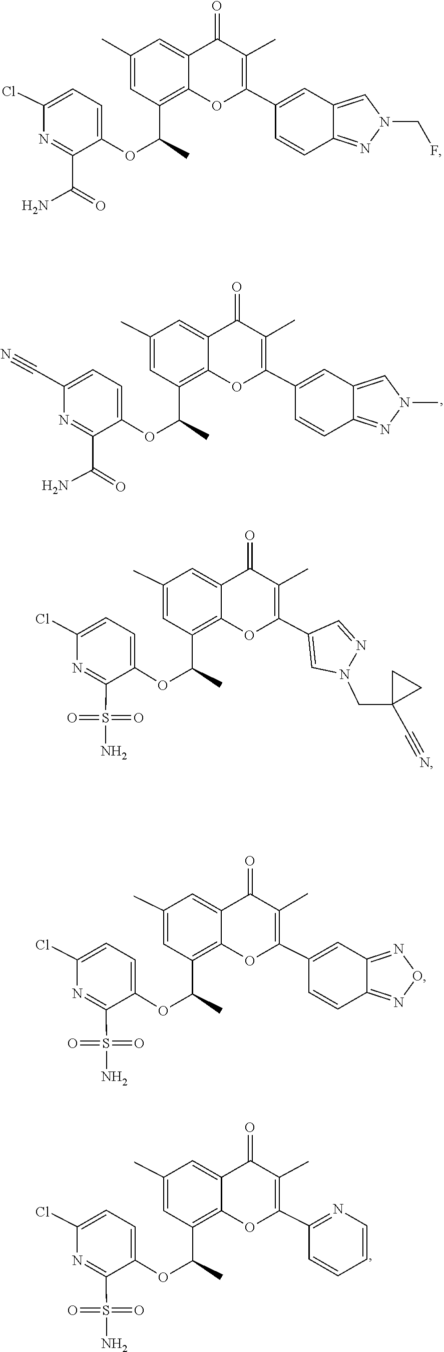 [SMILES: Cc1cc([C@@H](C)Oc2ccc(Cl)nc2C(N)=O)c2oc(-c3ccc4nn(CF)cc4c3)c(C)c(=O)c2c1 ; Cc1cc([C@@H](C)Oc2ccc(C#N)nc2C(N)=O)c2oc(-c3ccc4nn(C)cc4c3)c(C)c(=O)c2c1 ; Cc1cc([C@@H](C)Oc2ccc(Cl)nc2S(N)(=O)=O)c2oc(-c3cnn(CC4(C#N)CC4)c3)c(C)c(=O)c2c1 ; Cc1cc([C@@H](C)Oc2ccc(Cl)nc2S(N)(=O)=O)c2oc(-c3ccc4nonc4c3)c(C)c(=O)c2c1 ; Cc1cc([C@@H](C)Oc2ccc(Cl)nc2S(N)(=O)=O)c2oc(-c3ccccn3)c(C)c(=O)c2c1]

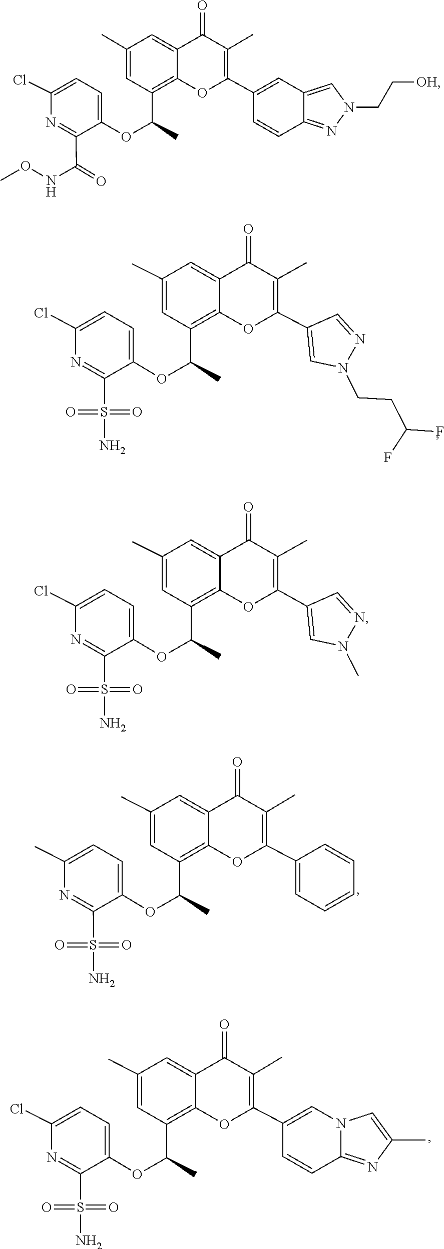 [SMILES: CONC(=O)c1nc(Cl)ccc1O[C@H](C)c1cc(C)cc2c(=O)c(C)c(-c3ccc4nn(CCO)cc4c3)oc12 ; Cc1cc([C@@H](C)Oc2ccc(Cl)nc2S(N)(=O)=O)c2oc(-c3cnn(CCC(F)F)c3)c(C)c(=O)c2c1 ; Cc1cc([C@@H](C)Oc2ccc(Cl)nc2S(N)(=O)=O)c2oc(-c3cnn(C)c3)c(C)c(=O)c2c1 ; Cc1cc([C@@H](C)Oc2ccc(C)nc2S(N)(=O)=O)c2oc(-c3ccccc3)c(C)c(=O)c2c1 ; Cc1cc([C@@H](C)Oc2ccc(Cl)nc2S(N)(=O)=O)c2oc(-c3ccc4nc(C)cn4c3)c(C)c(=O)c2c1]

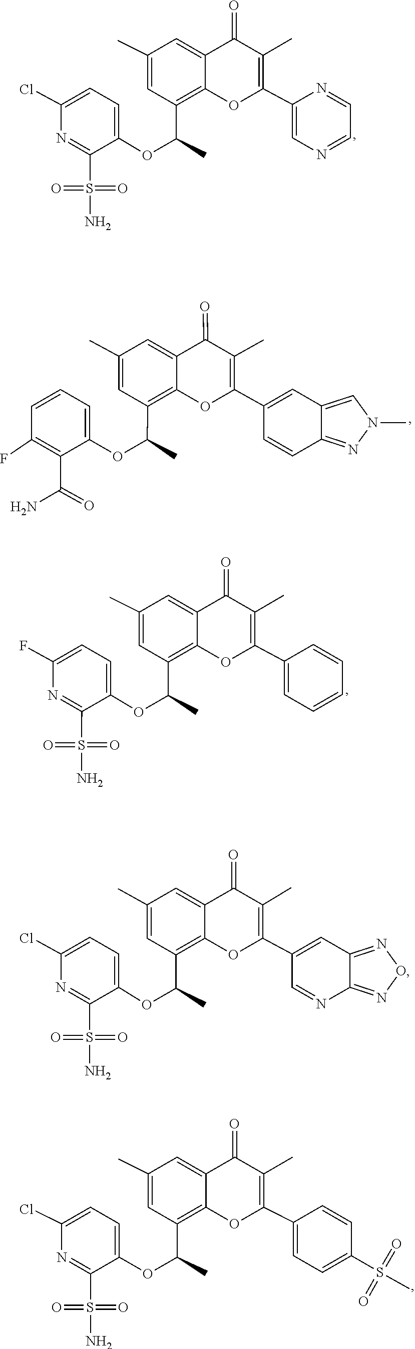 [SMILES: Cc1cc([C@@H](C)Oc2ccc(Cl)nc2S(N)(=O)=O)c2oc(-c3cnccn3)c(C)c(=O)c2c1 ; Cc1cc([C@@H](C)Oc2cccc(F)c2C(N)=O)c2oc(-c3ccc4nn(C)cc4c3)c(C)c(=O)c2c1 ; Cc1cc([C@@H](C)Oc2ccc(F)nc2S(N)(=O)=O)c2oc(-c3ccccc3)c(C)c(=O)c2c1 ; Cc1cc([C@@H](C)Oc2ccc(Cl)nc2S(N)(=O)=O)c2oc(-c3cnc4nonc4c3)c(C)c(=O)c2c1 ; Cc1cc([C@@H](C)Oc2ccc(Cl)nc2S(N)(=O)=O)c2oc(-c3ccc(S(C)(=O)=O)cc3)c(C)c(=O)c2c1]

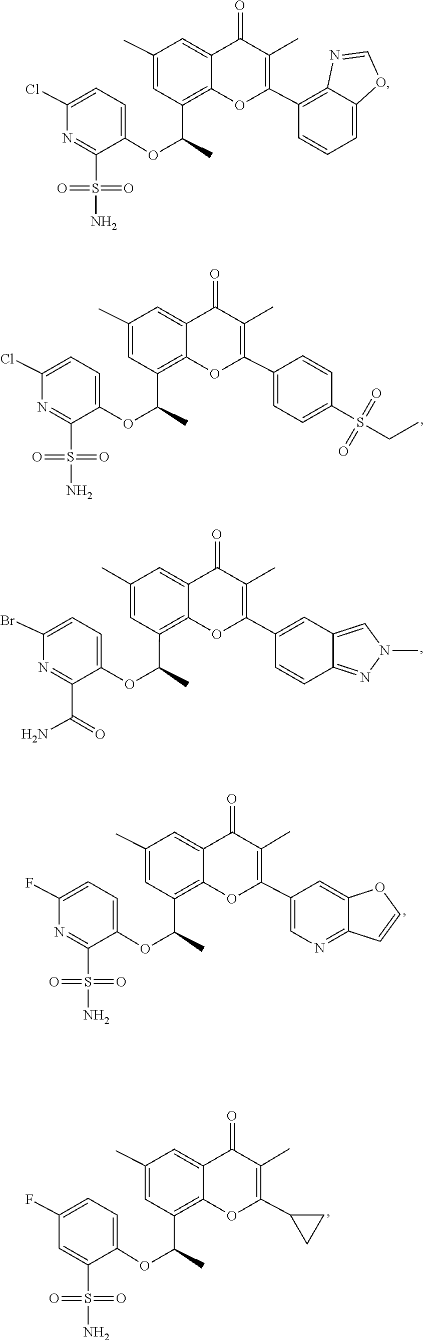 [SMILES: Cc1cc([C@@H](C)Oc2ccc(Cl)nc2S(N)(=O)=O)c2oc(-c3cccc4ocnc34)c(C)c(=O)c2c1 ; CCS(=O)(=O)c1ccc(-c2oc3c([C@@H](C)Oc4ccc(Cl)nc4S(N)(=O)=O)cc(C)cc3c(=O)c2C)cc1 ; Cc1cc([C@@H](C)Oc2ccc(Br)nc2C(N)=O)c2oc(-c3ccc4nn(C)cc4c3)c(C)c(=O)c2c1 ; Cc1cc([C@@H](C)Oc2ccc(F)nc2S(N)(=O)=O)c2oc(-c3cnc4ccoc4c3)c(C)c(=O)c2c1 ; Cc1cc([C@@H](C)Oc2ccc(F)cc2S(N)(=O)=O)c2oc(C3CC3)c(C)c(=O)c2c1]

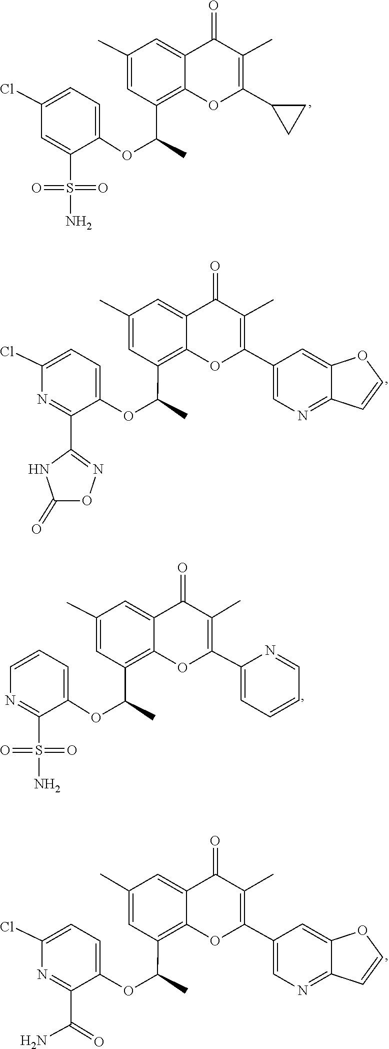 [SMILES: Cc1cc([C@@H](C)Oc2ccc(Cl)cc2S(N)(=O)=O)c2oc(C3CC3)c(C)c(=O)c2c1 ; Cc1cc([C@@H](C)Oc2ccc(Cl)nc2-c2noc(=O)[nH]2)c2oc(-c3cnc4ccoc4c3)c(C)c(=O)c2c1 ; Cc1cc([C@@H](C)Oc2cccnc2S(N)(=O)=O)c2oc(-c3ccccn3)c(C)c(=O)c2c1 ; Cc1cc([C@@H](C)Oc2ccc(Cl)nc2C(N)=O)c2oc(-c3cnc4ccoc4c3)c(C)c(=O)c2c1]

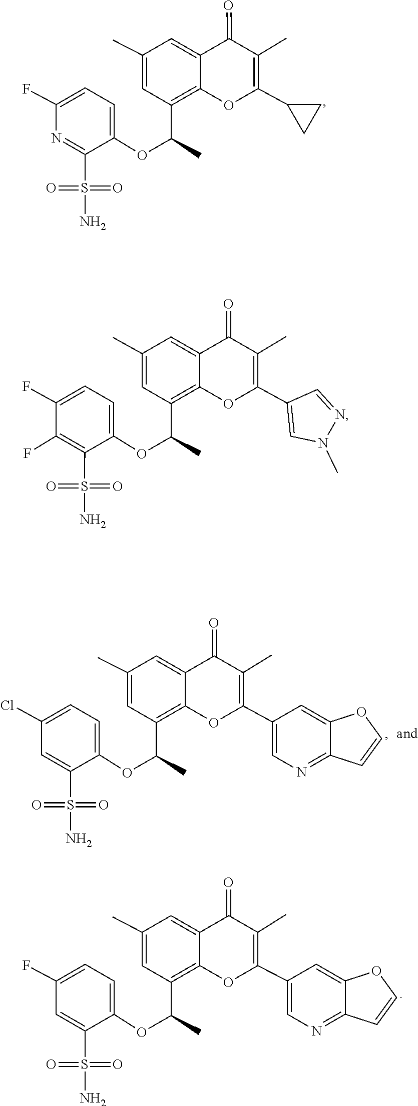 [SMILES: Cc1cc([C@@H](C)Oc2ccc(F)nc2S(N)(=O)=O)c2oc(C3CC3)c(C)c(=O)c2c1 ; Cc1cc([C@@H](C)Oc2ccc(F)c(F)c2S(N)(=O)=O)c2oc(-c3cnn(C)c3)c(C)c(=O)c2c1 ; Cc1cc([C@@H](C)Oc2ccc(Cl)cc2S(N)(=O)=O)c2oc(-c3cnc4ccoc4c3)c(C)c(=O)c2c1 ; Cc1cc([C@@H](C)Oc2ccc(F)cc2S(N)(=O)=O)c2oc(-c3cnc4ccoc4c3)c(C)c(=O)c2c1]

26. A pharmaceutical composition comprising a compound of claim 1, or a pharmaceutically acceptable salt thereof, and a pharmaceutically acceptable carrier.

27. A method of treating a disease or disorder associated with modulation of phosphoinositide 3-kinase (PI3K), comprising administering to a patient in need thereof a therapeutically effective amount of a compound of claim 1, or a pharmaceutically acceptable salt thereof.

28. The method of claim 27, wherein the PI3K is PI3Kα.

29. The method of claim 28, wherein the PI3K associated with the disease or disorder has a H1047R mutation, an E575K mutation, or both of a H1047R mutation and an E575K mutation.

30. The method of claim 27, wherein the disease or disorder is a cancer.

31. The method of claim 30, wherein the cancer is endometrial cancer, gastric cancer, leukemia, lymphoma, sarcoma, colorectal cancer, lung cancer, ovarian cancer, skin cancer, head and neck cancer, breast cancer, brain cancer, or prostate cancer.

32. The method of claim 30, wherein the cancer is breast cancer.

33. The method of claim 30, wherein the cancer is hormone receptor-positive (HR+), human epidermal growth factor receptor 2-negative (HER2−) advanced or metastatic breast cancer.

34. The method of claim 27, wherein the disorder is CLOVES syndrome (congenital lipomatous overgrowth, vascular malformations, epidermal naevi, scoliosis/skeletal, and spinal syndrome), or PIK3CA-related overgrowth syndrome (PROS).

35. A method of inhibiting phosphoinositide 3-kinase (PI3K), comprising administering to a patient in need thereof a therapeutically effective amount of a compound of claim 1, or a pharmaceutically acceptable salt thereof.

36. A method of treating cancer or a disorder associated with modulation of phosphoinositide 3-kinase (PI3K), the method comprising administering to a patient in need thereof a therapeutically effective amount of a compound of claim 1, or a pharmaceutically acceptable salt thereof.

37. The method of claim 36, wherein the cancer is endometrial cancer, gastric cancer, leukemia, lymphoma, sarcoma, colorectal cancer, lung cancer, ovarian cancer, skin cancer, head and neck cancer, breast cancer, brain cancer, or prostate cancer.

38. The method of claim 36, wherein the cancer is breast cancer.

39. The method of claim 36, wherein the cancer is hormone receptor-positive (HR+), human epidermal growth factor receptor 2-negative (HER2−) advanced or metastatic breast cancer.

40. The method of claim 36, wherein the disorder is CLOVES syndrome or PROS.

41.-53. (canceled)
